(19) United States
(12) Patent Application Publication
(10) Pub. No.: US 2026/0040818 A1
(43) Pub. Date: Feb. 5, 2026

(54) PLURALITY OF HOST MATERIALS, ORGANIC ELECTROLUMINESCENT COMPOUND, AND ORGANIC ELECTROLUMINESCENT DEVICE COMPRISING THE SAME

(71) Applicant: DuPont Specialty Materials Korea Ltd., Chungcheongnam-do (KR)

(72) Inventors: Jeong-Eun YANG, Gyeonggi-do (KR); Ga-Won LEE, Gyeonggi-do (KR); Hee-Ryong KANG, Gyeonggi-do (KR); Sang-Hee CHO, Gyeonggi-do (KR); Jeong-Hwan JEON, Gyeonggi-do (KR); Hyo-Soon PARK, Gyeonggi-do (KR)

(21) Appl. No.: 18/678,479

(22) Filed: May 30, 2024

(30) Foreign Application Priority Data
Jun. 15, 2023 (KR) 10-2023-0076531

(51) Int. Cl. H10K 85/60; C07D 413/10; C07D 413/12; C07D 413/14; C09K 11/02; H10K 50/11; H10K 101/00

(52) U.S. Cl. CPC H10K 85/654; C07D 413/10; C07D 413/12; C07D 413/14; C09K 11/02; H10K 85/636; H10K 50/11; H10K 85/657; H10K 85/6574; H10K 2101/90

(57) ABSTRACT

The present disclosure relates to a plurality of host materials, an organic electroluminescent compound, and an organic electroluminescent device. By comprising a plurality of host materials and/or an organic electroluminescent compound according to the present disclosure, an organic electroluminescent device can be provided which has low driving voltage and/or high luminous efficiency and/or long lifespan characteristics.

### TECHNICAL FIELD

The present disclosure relates to a plurality of host materials, an organic electroluminescent compound, and an organic electroluminescent device comprising the same.

### BACKGROUND ART

The TPD/Alq₃ bilayer small molecule organic electroluminescent device (OLED) with green emission, which is constituted with a light-emitting layer and a charge transport layer, was first developed by Tang et al. of Eastman Kodak in 1987. Thereafter, studies on organic electroluminescent devices have proceeded rapidly, and OLEDs have since been commercialized. At present, OLEDs primarily use phosphorescent materials having excellent luminous efficiency in panel implementation. Therefore, an OLED having high luminous efficiency and/or long lifespan characteristics is required for long-term use and high display resolution.

Various materials or concepts have been proposed for the organic layer of an organic electroluminescent device in order to improve luminous efficiency, driving voltage, and/or lifespan, but these have not been satisfactory for practical use. Accordingly, there is a continuous need to develop organic electroluminescent devices with improved performance, such as improved driving voltage, luminous efficiency, power efficiency, and/or lifespan characteristics compared to previously disclosed organic electroluminescent devices.

Chinese Patent Application Laid-open No. 114702484 discloses an organic electroluminescent device containing an azole-based organic compound as a single host, but does not specifically disclose a plurality of host materials containing the specific combination of compounds as described in the present disclosure.

### DISCLOSURE OF THE INVENTION

### Technical Problem

The object of the present disclosure is, firstly, to provide a plurality of host materials capable of producing an organic electroluminescent device having low driving voltage and/or high luminous efficiency and/or long lifespan characteristics and, secondly, to provide an organic electroluminescent compound having a novel structure suitable for use as an organic electroluminescent device. In addition, another object of the present disclosure is to provide an organic electroluminescent device with low driving voltage and/or high luminous efficiency and/or long lifespan characteristics by comprising a specific combination of compounds or an organic electroluminescent compound according to the present disclosure.

### Solution to Problem

As a result of intensive studies to solve the technical problem above, the present inventors found that the aforementioned objective can be achieved by a plurality of host materials comprising at least one first host compound represented by the following Formula 1 and at least one second host compound represented by the following Formula 2, thereby completing the present invention.

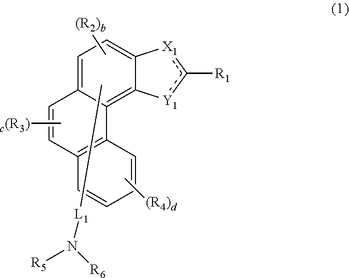

In Formula 1,
X₁ and Y₁ each independently represent —N=, —NR₇, —O—, or —S—; provided that any one of X₁ and Y₁ is —N=, and the other of X₁ and Y₁ is —NR₇—, —O—, or —S—;
R₁ represents a substituted or unsubstituted (C₆-C₃₀)aryl or a substituted or unsubstituted (3- to 30-membered)heteroaryl;
R₂ to R₄ and R₇ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, or —L₁₁-N(Ar₁)(Ar₂); or may be linked to the adjacent substituents to form a ring(s);
L₁ and L₁₁ each independently represent a single bond, a substituted or unsubstituted (C₆-C₃₀)arylene, or a substituted or unsubstituted (3- to 30-membered)heteroarylene;
R₅ and R₆ each independently represent a substituted or unsubstituted (C₆-C₃₀)aryl or a substituted or unsubstituted (3- to 30-membered)heteroaryl;
Ar₁ and Ar₂ each independently represent hydrogen, a substituted or unsubstituted (C₁-C₃₀)alkyl, a substituted or unsubstituted (C₂-C₃₀)alkenyl, a substituted or unsubstituted fused ring of (C₃-C₃₀) aliphatic ring and (C₆-C₃₀) aromatic ring, a substituted or unsubstituted (C₆-C₃₀)aryl, or a substituted or unsubstituted (3- to 30-membered)heteroaryl; and
b and c each independently represent an integer of 1 or 2, d is an integer of 1 to 4, and when b to d are an integer of 2 or more, each of R₂ to R₄ may be the same or different.

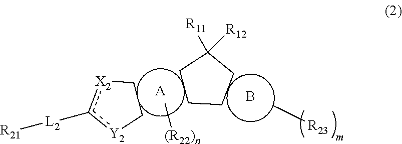

In Formula 2,
ring A and ring B each independently represent a substituted or unsubstituted (C₆-C₃₀)arene or a substituted or unsubstituted (3- to 30-membered)heteroarene;
R₁₁ and R₁₂ each independently represent a substituted or unsubstituted (C₁-C₃₀)alkyl, a substituted or unsubstituted (C₆-C₃₀)aryl, or a substituted or unsubstituted (3- to 30-membered)heteroaryl; or may be linked to each other to form a ring(s);
X₂ and Y₂ each independently represent —N=, —NR₂₄, —O—, or —S—; provided that any one of X₂ and Y₂ is —N=, and the other of X₂ and Y₂ is —NR₂₄—, —O—, or —S—;
R₂₁ to R₂₄ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and a (C₆-C₃₀) aromatic ring, or —L₃-HAr;
provided that at least one of R₂₁ to R₂₄ is —L₃-HAr;
L₂ and L₃ each independently represent a single bond, a substituted or unsubstituted (C₆-C₃₀)arylene, or a substituted or unsubstituted (3- to 30-membered)heteroarylene;
HAr represents a substituted or unsubstituted (3- to 30-membered)heteroaryl containing at least one nitrogen atom; and
n represents an integer of 1 to 4, m represents an integer of 1 to 8, and when n and m are an integer of 2 or more, each of R₂₂ and R₂₃ may be the same or different.

### Advantageous Effects of Invention

By comprising a plurality of host materials and/or an organic electroluminescent compound according to the present disclosure, an organic electroluminescent device having low driving voltage and/or high luminous efficiency and/or long lifespan properties can be provided.

### EMBODIMENTS OF THE INVENTION

Hereinafter, the present disclosure will be described in detail. However, the following description is intended to explain the invention, and is not meant in any way to restrict the scope of the invention.

The present disclosure relates to a plurality of host materials comprising at least one first host compound(s) represented by Formula 1 and at least one second host compound(s) represented by Formula 2, and an organic electroluminescent device comprising the same.

The present disclosure relates to an organic electroluminescent compound represented by Formula 2′, an organic electroluminescent material comprising the organic electroluminescent compound, and an organic electroluminescent device comprising the organic electroluminescent compound and/or the organic electroluminescent material.

Herein, The term “organic electroluminescent compound” in the present disclosure means a compound that may be used in an organic electroluminescent device, and may be comprised in any material layer constituting an organic electroluminescent device, as necessary.

Herein, the term “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron-blocking material, a light-emitting material (containing host and dopant materials), an electron buffer material, a hole-blocking material, an electron transport material, or an electron injection material, etc.

The term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron-blocking layer, a light-emitting layer, an electron buffer layer, a hole-blocking layer, an electron transport layer, and an electron injection layer. As such, at least two compounds may be comprised in the same layer or in different layers, and may be mixture-evaporated or co-evaporated, or may be individually evaporated.

Herein, the term “a plurality of host materials” means an organic electroluminescent material comprising a combination of at least two host materials. It may mean both a material before being comprised in an organic electroluminescent device (e.g., before vapor deposition) and a material after being comprised in an organic electroluminescent device (e.g., after vapor deposition). A plurality of host materials of the present disclosure may be comprised in any light-emitting layer constituting an organic electroluminescent device. The at least two compounds comprised in a plurality of host materials may be comprised together in one light-emitting layer, or may each be comprised in separate light-emitting layers. When at least two compounds are comprised in one light-emitting layer, the at least two compounds may be mixture-evaporated to form a layer or may be individually and simultaneously co-evaporated to form a layer.

Herein, “(C₁-C₃₀)alkyl(ene)” is meant to be a linear or branched alkyl having 1 to 30 carbon atoms constituting the chain, in which the number of carbon atoms is preferably 1 to 20, and more preferably 1 to 10. The above alkyl may include methyl, ethyl, n-propyl, isopropyl, n-butyl, isobutyl, tert-butyl, sec-butyl, etc. Herein, the term “(C₃-C₃₀)cycloalkyl(ene)” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. Herein, “(C₆-C₃₀)aryl(ene)” is a monocyclic or fused ring radical derived from an aromatic hydrocarbon having 6 to 30 ring backbone carbon atoms, in which the number of the ring backbone carbon atoms is preferably 6 to 20, more preferably 6 to 15, may be partially saturated, and may include a spiro structure. Examples of the aryl specifically may be phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluoren-fluoren]yl, spiro[fluoren-benzofluoren]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. Herein, “(3- to 30-membered)heteroaryl(ene)” is an aryl having 3 to 30 ring backbone atoms and including at least one, preferably 1 to 4 heteroatom(s) selected from the group consisting of B, N, O, S, Si, P, Se, and Ge, in which the number of the ring backbone carbon atoms is preferably 5 to 25. The above heteroaryl(ene) may be a monocyclic ring, or a fused ring condensed with at least one benzene ring; and may be partially saturated. Also, the above heteroaryl herein may be one formed by linking at least one heteroaryl or aryl group to a heteroaryl group via a single bond(s). Examples of the heteroaryl specifically may be a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Herein, the term “a fused ring of (C₃-C₃₀) aliphatic ring and (C₆-C₃₀) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C₃-C₃₀) aliphatic ring and (C₆-C₃₀) aromatic ring may be replaced with at least one heteroatom selected from B, N, O, S, Si, and P, preferably at least one heteroatom selected from N, O, and S. The term “halogen” in the present disclosure includes F, Cl, Br, and I.

In addition, “ortho-” (“o-”), “meta-” (“m-”), and “para-” (“p-”) are meant to signify the substitution position of all substituents. An ortho-configuration describes a compound with substituents which are adjacent to each other, e.g., at the 1 and 2 positions on benzene. A meta-configuration indicates the next substitution position of the immediately adjacent substitution position, e.g., a compound with substituents at the 1 and 3 positions on benzene. A para-configuration indicates the next substitution position from the meta-position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, preferably may be a substituted or unsubstituted (3- to 26-membered) mono- or polycyclic, alicyclic, aromatic ring, or a combination thereof. Further, the formed ring may include at least one heteroatom selected from the group consisting of B, N, O, S, Si, and P, preferably, N, O, and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, “substituted” in the expression “substituted or unsubstituted” means that a hydrogen atom in a certain functional group is replaced with another atom or functional group (i.e., a substituent), and also includes substituted with a group in which two or more of the above substituents are connected among the substituents. For example, “a substituent to which two or more substituents are connected” may be pyridine-triazine. That is, pyridine-triazine may be a heteroaryl or may be interpreted as one substituent in which two heteroaryls are connected. The substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, and the substituted fused ring of aliphatic ring and aromatic ring in the formulas of the present disclosure, each independently may be substituted with at least one selected from the group consisting of deuterium; halogen; cyano; carboxyl; nitro; hydroxyl; (C₁-C₃₀)alkyl; halo(C₁-C₃₀)alkyl; (C₂-C₃₀)alkenyl; (C₂-C₃₀)alkynyl; (C₁-C₃₀)alkoxy; (C₁-C₃₀)alkylthio; (C₃-C₃₀)cycloalkyl; (C₃-C₃₀)cycloalkenyl; (3- to 7-membered)heterocycloalkyl; (C₆-C₃₀)aryloxy; (C₆-C₃₀)arylthio; (3- to 30-membered)heteroaryl unsubstituted or substituted with at least one (C₆-C₃₀)aryl; (C₆-C₃₀)aryl unsubstituted or substituted with at least one of (C₁-C₃₀)alkyl and (3- to 30-membered)heteroaryl; tri(C₁-C₃₀)alkylsilyl; tri(C₆-C₃₀)arylsilyl; di(C₁-C₃₀)alkyl(C₆-C₃₀)arylsilyl; (C₁-C₃₀)alkyldi(C₆-C₃₀)arylsilyl; tri(C₆-C₃₀)arylgermanyl; amino; mono- or di(C₁-C₃₀)alkylamino; mono- or di(C₂-C₃₀)alkenylamino; mono- or di(C₆-C₃₀)arylamino unsubstituted or substituted with (C₁-C₃₀)alkyl; mono- or di(3- to 30-membered)heteroarylamino; (C₁-C₃₀)alkyl(C₂-C₃₀)alkenylamino; (C₁-C₃₀)alkyl(C₆-C₃₀)arylamino; (C₁-C₃₀)alkyl(3- to 30-membered)heteroarylamino; (C₂-C₃₀)alkenyl(C₆-C₃₀)arylamino; (C₂-C₃₀)alkenyl(3- to 30-membered)heteroarylamino; (C₆-C₃₀)aryl(3- to 30-membered)heteroarylamino; (C₁-C₃₀)alkylcarbonyl; (C₁-C₃₀)alkoxycarbonyl; (C₆-C₃₀)arylcarbonyl; di(C₆-C₃₀)arylboronyl; di(C₁-C₃₀)alkylboronyl; (C₁-C₃₀)alkyl(C₆-C₃₀)arylboronyl; (C₆-C₃₀)ar(C₁-C₃₀)alkyl; and (C₁-C₃₀)alkyl(C₆-C₃₀)aryl, etc.

In the formulas of the present disclosure, when a plurality of substituents represented by the same symbol are present, each of these substituents, represented by the same symbol, may be the same or different.

Hereinafter, the plurality of host materials according to one embodiment will be described.

The plurality of host materials according to one embodiment comprise at least one first host compound and at least one second host compound, wherein the first host compound is represented by Formula 1 and the second host compound is represented by Formula 2.

The first host compound as the host material according to one embodiment may be represented by the following Formula 1.

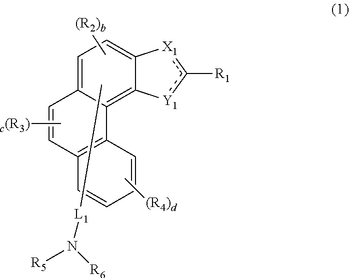

In Formula 1,
X₁ and Y₁ each independently represent —N=, —NR₇, —O—, or —S—; provided that any one of X₁ and Y₁ is —N=, and the other of X₁ and Y₁ is —NR₇—, —O—, or —S—;
R₁ represents a substituted or unsubstituted (C₆-C₃₀)aryl or a substituted or unsubstituted (3- to 30-membered)heteroaryl;
R₂ to R₄ and R₇ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, or —L₁₁-N(Ar₁)(Ar₂); or may be linked to the adjacent substituents to form a ring(s);
L₁ and L₁₁ each independently represent a single bond, a substituted or unsubstituted (C₆-C₃₀)arylene, or a substituted or unsubstituted (3- to 30-membered)heteroarylene;
R₅ and R₆ each independently represent a substituted or unsubstituted (C₆-C₃₀)aryl or a substituted or unsubstituted (3- to 30-membered)heteroaryl;
Ar₁ and Ar₂ each independently represent hydrogen, a substituted or unsubstituted (C₁-C₃₀)alkyl, a substituted or unsubstituted (C₂-C₃₀)alkenyl, a substituted or unsubstituted fused ring of (C₃-C₃₀) aliphatic ring and (C₆-C₃₀) aromatic ring, a substituted or unsubstituted (C₆-C₃₀)aryl, or a substituted or unsubstituted (3- to 30-membered)heteroaryl; and
b and c each independently represent an integer of 1 or 2, d represents an integer of 1 to 4, and when b to d are an integer of 2 or more, each of R₂ to R₄ may be the same or different.

According to one embodiment, Formula 1 may be represented by any one of the following Formulas 1-1 to 1-4.

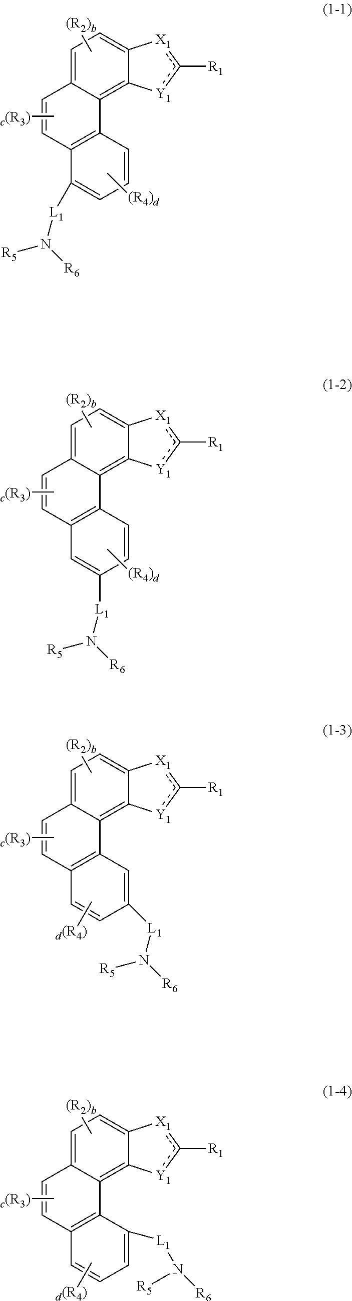

In Formulas 1-1 to 1-4,
d represents an integer of 1 to 3, and when d is an integer of 2 or more, each of R₄ may be the same or different; and
X₁, Y₁, R₁ to R₆, L₁, b, and c are as defined in Formula 1.

In one embodiment, any one of X₁ and Y₁ may be —N=, and the other of X₁ and Y₁ may be —O— or —S—. For example, X₁ may be —N= and Y₁ may be —O—, or X₁ may be —O— and Y₁ may be —N=, or X₁ may be —S— and Y₁ may be —N=.

In one embodiment, R₁ may be a substituted or unsubstituted (C₆-C₃₀)aryl or a substituted or unsubstituted (5- to 30-membered)heteroaryl, preferably a substituted or unsubstituted (C₆-C₂₅)aryl or a substituted or unsubstituted (5- to 25-membered)heteroaryl, more preferably a substituted or unsubstituted (C₆-C₁₈)aryl or a substituted or unsubstituted (5- to 18-membered)heteroaryl. For example, R₁ may be unsubstituted phenyl, unsubstituted naphthyl, unsubstituted o-biphenyl, unsubstituted m-biphenyl, unsubstituted p-biphenyl, or unsubstituted pyridyl.

In one embodiment, R₂ to R₄ and R₇ each independently may be hydrogen, deuterium, halogen, cyano, a substituted or unsubstituted (C₁-C₃₀)alkyl, or a substituted or unsubstituted (C₆-C₃₀)aryl, preferably hydrogen, deuterium, halogen, cyano, or a substituted or unsubstituted (C₆-C₂₅)aryl, more preferably hydrogen, deuterium, or a substituted or unsubstituted (C₆-C₁₈)aryl. For example, R₂ to R₄ each independently may be hydrogen or unsubstituted phenyl.

In one embodiment, L, may be a single bond or a substituted or unsubstituted (C₆-C₃₀)arylene, preferably a single bond or a substituted or unsubstituted (C₆-C₂₅)arylene, more preferably a substituted or unsubstituted (C₆-C₁₈)arylene. For example, L, may be a single bond, or unsubstituted phenylene, or unsubstituted naphthylene.

In one embodiment, R₅ and R₆ each independently may be a substituted or unsubstituted (C₆-C₃₀)aryl, a substituted or unsubstituted (5- to 30-membered)heteroaryl, or —L₁₁-N(Ar₁)(Ar₂), preferably a substituted or unsubstituted (C₆-C₂₅)aryl, a substituted or unsubstituted (5- to 25-membered)heteroaryl, or —L₁₁-N(Ar₁)(Ar₂), more preferably a substituted or unsubstituted (C₆-C₂₅)aryl, a substituted or unsubstituted (5- to 20-membered)heteroaryl, or —L₁₁-N(Ar₁)(Ar₂). For example, R₅ and R₆ each independently may be a substituted or unsubstituted phenyl, a substituted or unsubstituted o-biphenyl, a substituted or unsubstituted m-biphenyl, a substituted or unsubstituted p-biphenyl, a substituted or unsubstituted naphthyl, a substituted or unsubstituted p-terphenyl, a substituted or unsubstituted m-terphenyl, a substituted or unsubstituted o-terphenyl, a substituted or unsubstituted o-quaterphenyl, a substituted or unsubstituted fluorenyl, a substituted or unsubstituted phenanthrenyl, a substituted or unsubstituted benzofuranyl, a substituted or unsubstituted dibenzofuranyl, a substituted or unsubstituted dibenzothiophenyl, a substituted or unsubstituted benzofluorenyl, a substituted or unsubstituted spirobifluorenyl, a substituted or unsubstituted C₂₂ aryl, a substituted or unsubstituted carbazolyl, a substituted or unsubstituted benzonaphthofuranyl, or a substituted or unsubstituted benzofuropyridyl. The substituents in the substituted groups may be substituted with at least one of deuterium; methyl; tert-butyl; cyclohexyl; phenyl unsubstituted or substituted with at least one of methyl and tert-butyl; naphthyl; biphenyl; anthracenyl; fluoranthenyl; phenylfluorenyl; pyridyl unsubstituted or substituted with phenyl; phenoxazinyl; diphenylamino; benzimidazolyl substituted with phenyl; triphenylsilane; diphenylnaphthylsilane; and biphenyldiphenylsilane.

In one embodiment, L₁₁ may be a single bond or a substituted or unsubstituted (C₆-C₃₀)arylene, preferably a single bond or a substituted or unsubstituted (C₆-C₂₅)arylene, more preferably a substituted or unsubstituted (C₆-C₁₈)arylene. For example, L₁₁ may be unsubstituted phenylene.

In one embodiment, Ar₁ and Ar₂ each independently may be a substituted or unsubstituted (C₆-C₃₀)aryl, preferably a substituted or unsubstituted (C₆-C₂₅)aryl, more preferably a substituted or unsubstituted (C₆-C₁₈)aryl. For example, Ar₁ and Ar₂ each independently may be a substituted or unsubstituted phenyl.

According to one embodiment, the first host compound represented by Formula 1 may be more specifically illustrated by the following compounds, but is not limited thereto.

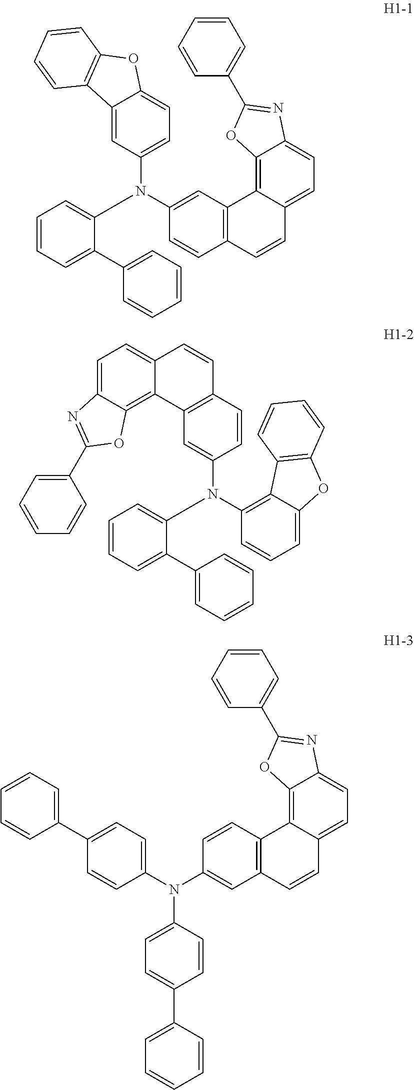

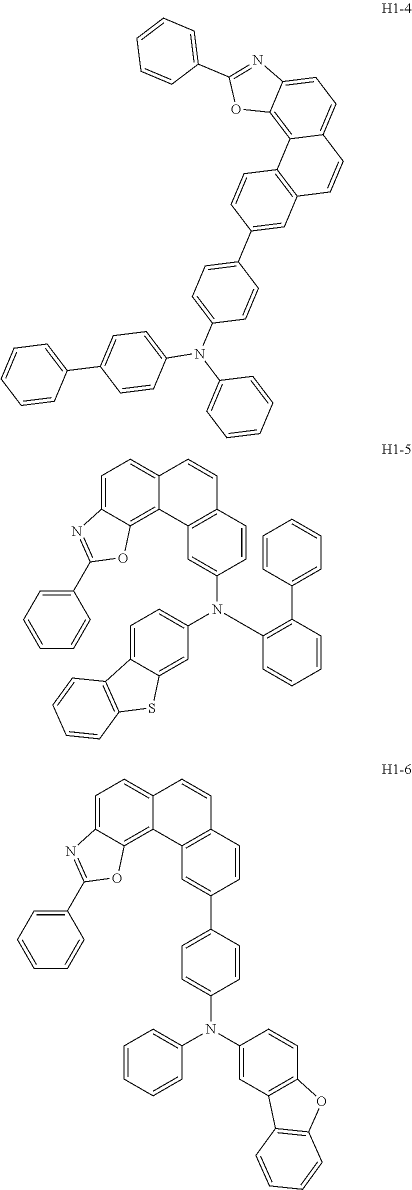

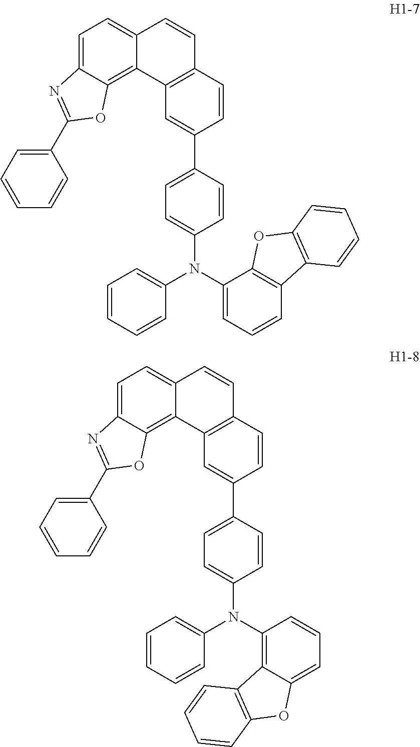

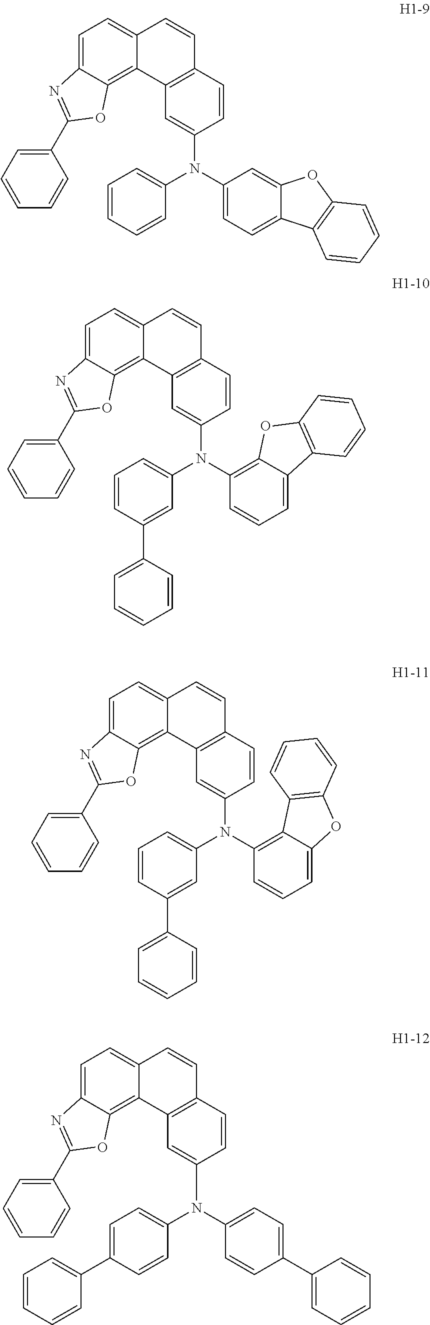 [SMILES: c1ccc(-c2nc3ccc4ccc5ccc(N(c6ccccc6)c6ccc7c(c6)oc6ccccc67)cc5c4c3o2)cc1 ; c1ccc(-c2cccc(N(c3ccc4ccc5ccc6nc(-c7ccccc7)oc6c5c4c3)c3cccc4c3oc3ccccc34)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6nc(-c7ccccc7)oc6c5c4c3)cc2)cc1]

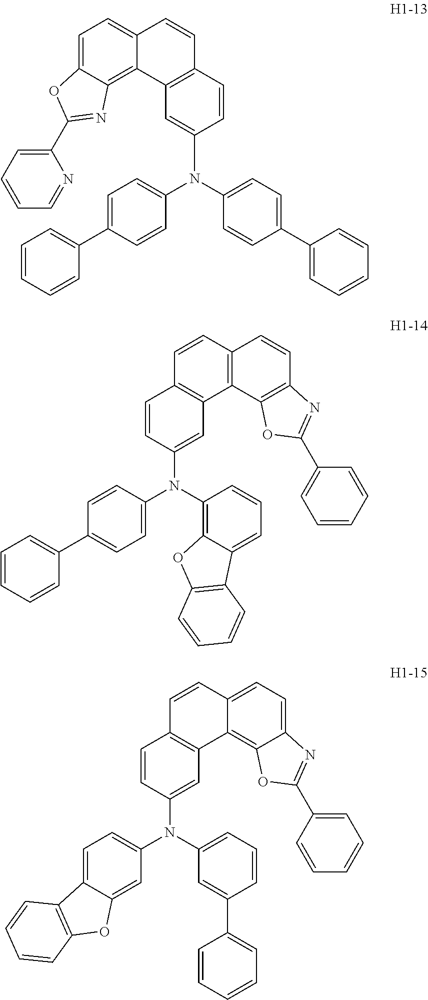

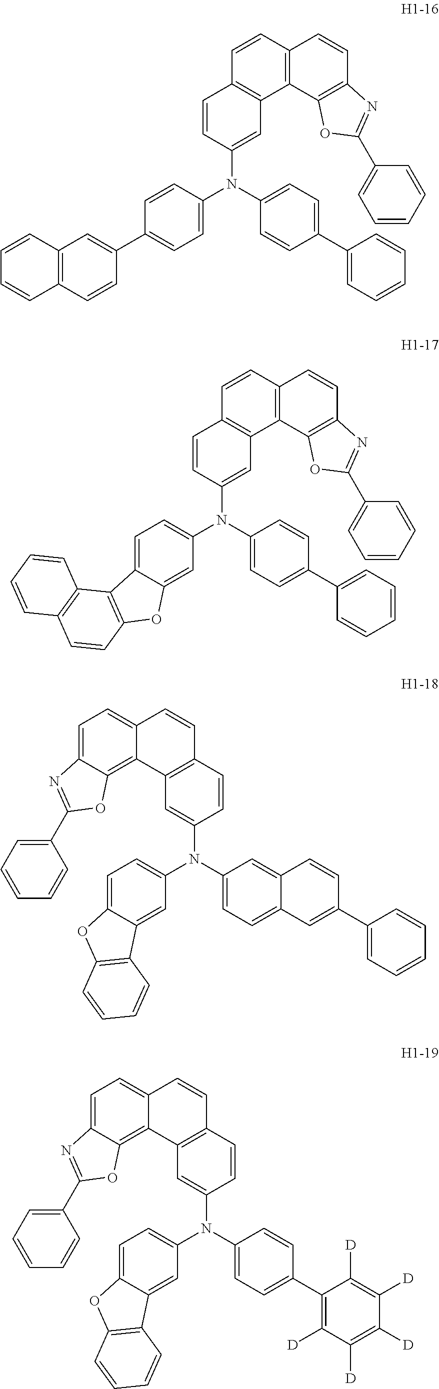 [SMILES: c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1 ; [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H]]

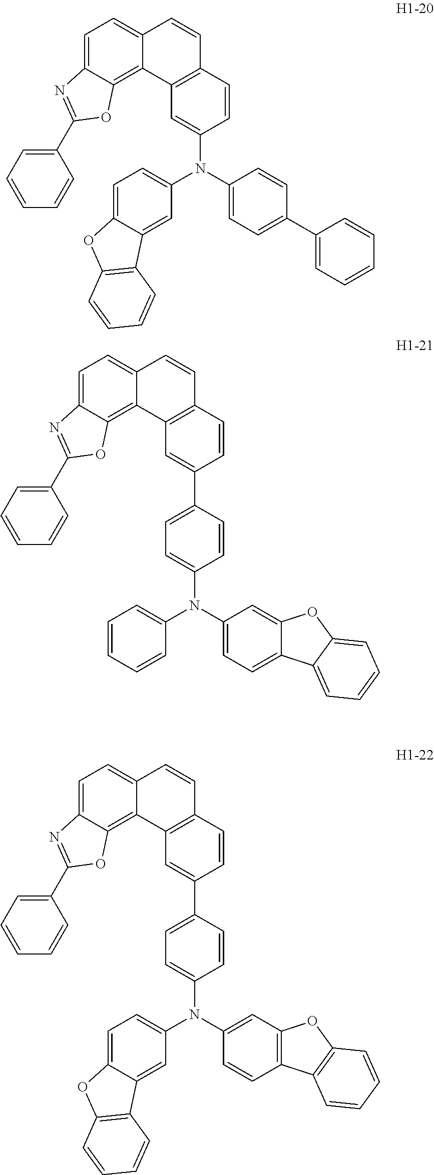

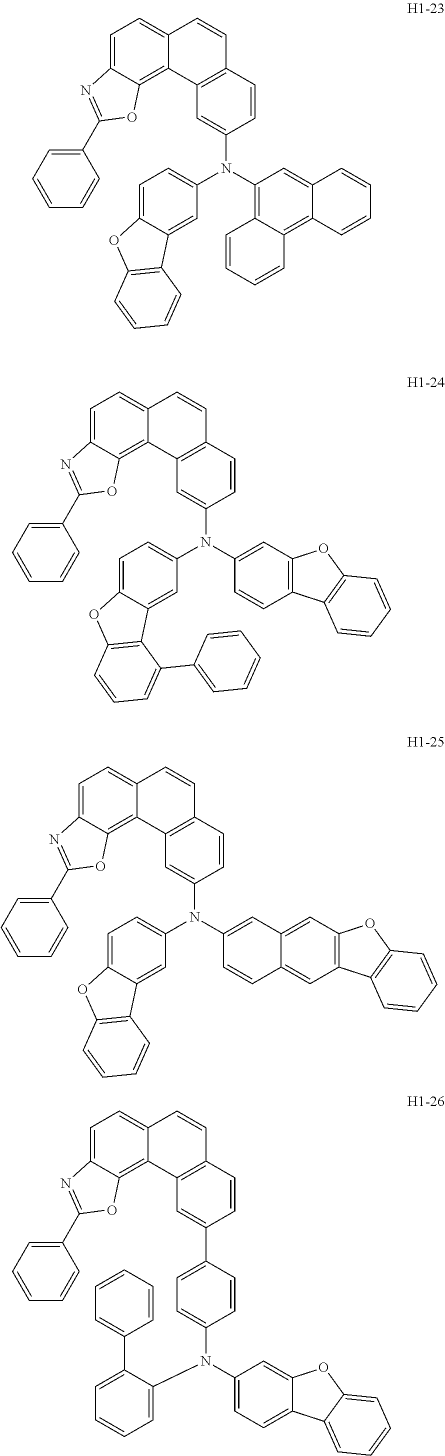

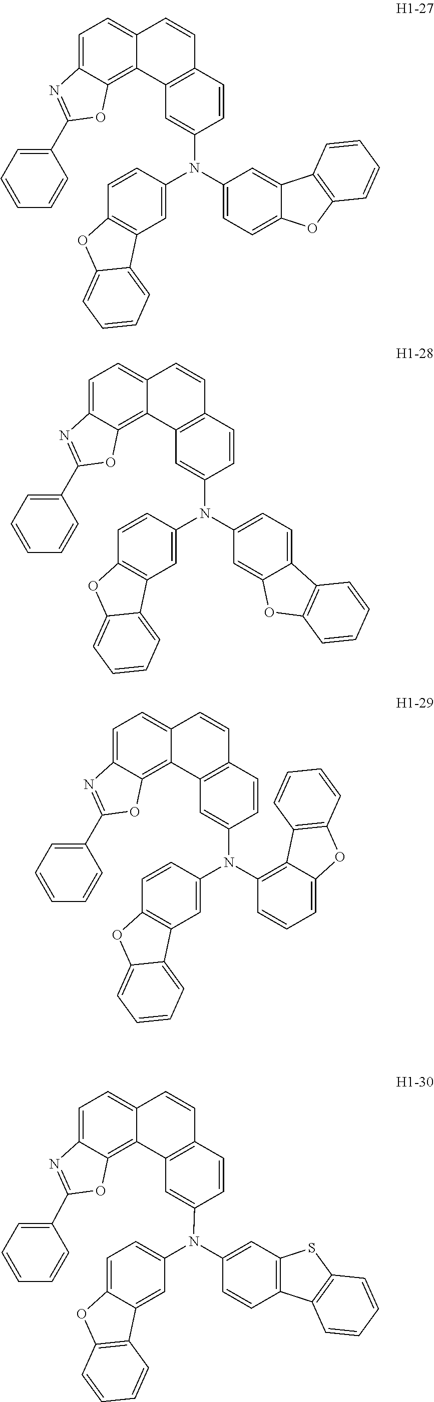

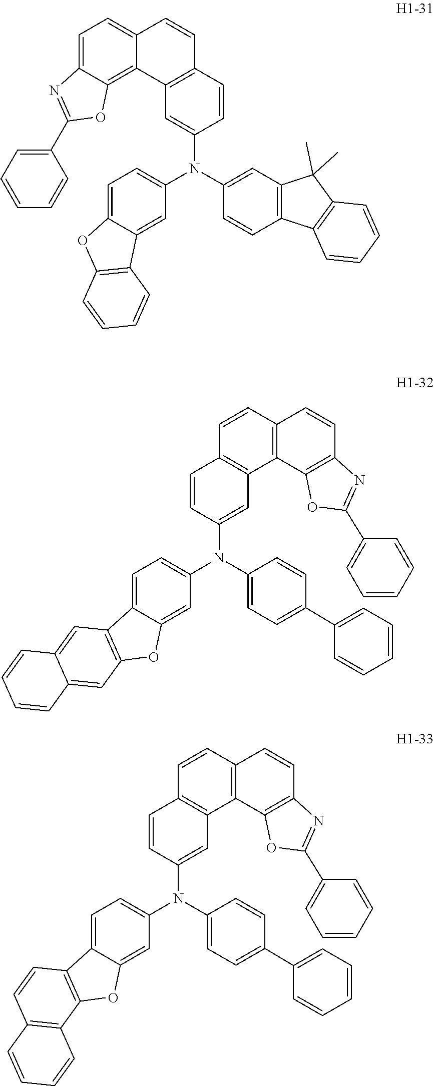 [SMILES: CC1(C)c2ccccc2-c2ccc(N(c3ccc4oc5ccccc5c4c3)c3ccc4ccc5ccc6nc(-c7ccccc7)oc6c5c4c3)cc21 ; c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1]

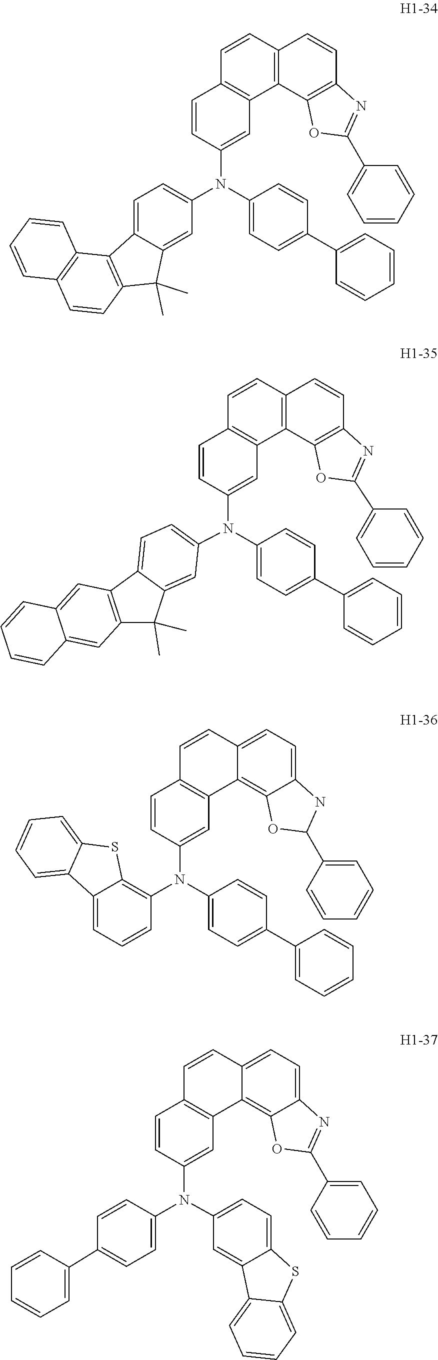 [SMILES: CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6c(c5c4c3)OC(c3ccccc3)N6)c3cccc4c3sc3ccccc34)cc2)cc1 ; c1ccc(-c2ccc(N(c3ccc4sc5ccccc5c4c3)c3ccc4ccc5ccc6nc(-c7ccccc7)oc6c5c4c3)cc2)cc1]

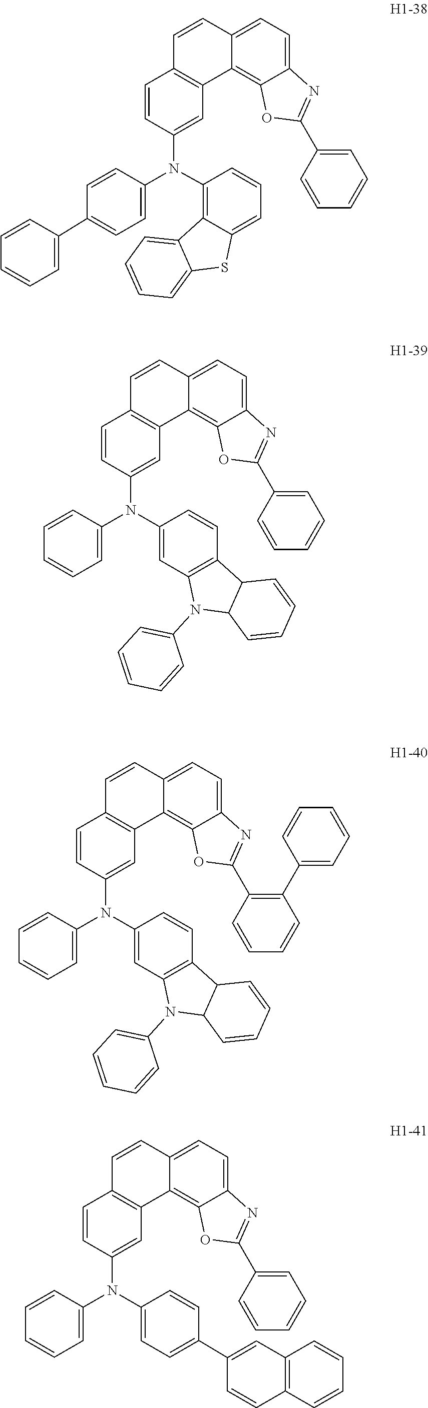 [SMILES: c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc3ccc4ccc5ccc(N(c6ccccc6)c6ccc(-c7ccc8ccccc8c7)cc6)cc5c4c3o2)cc1]

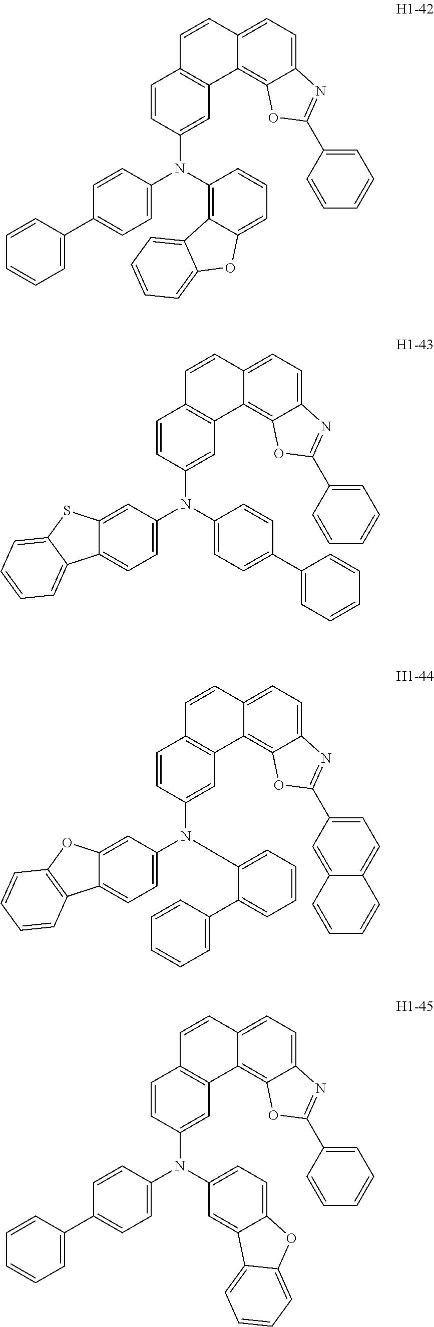

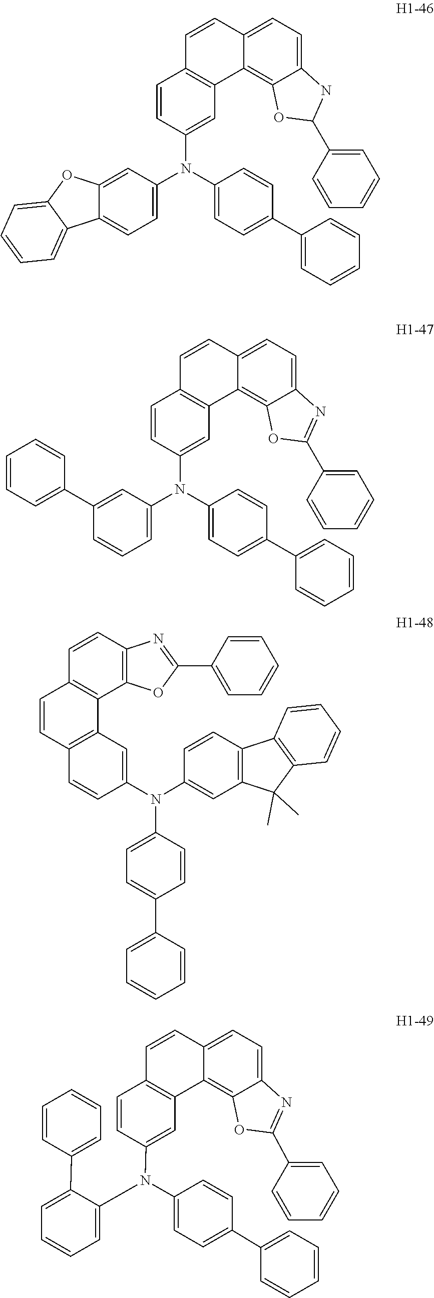

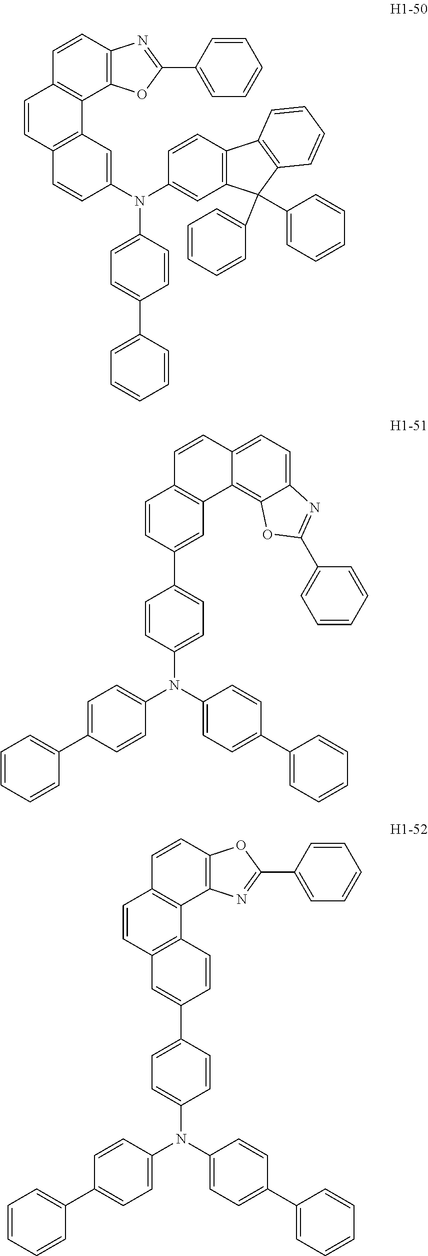

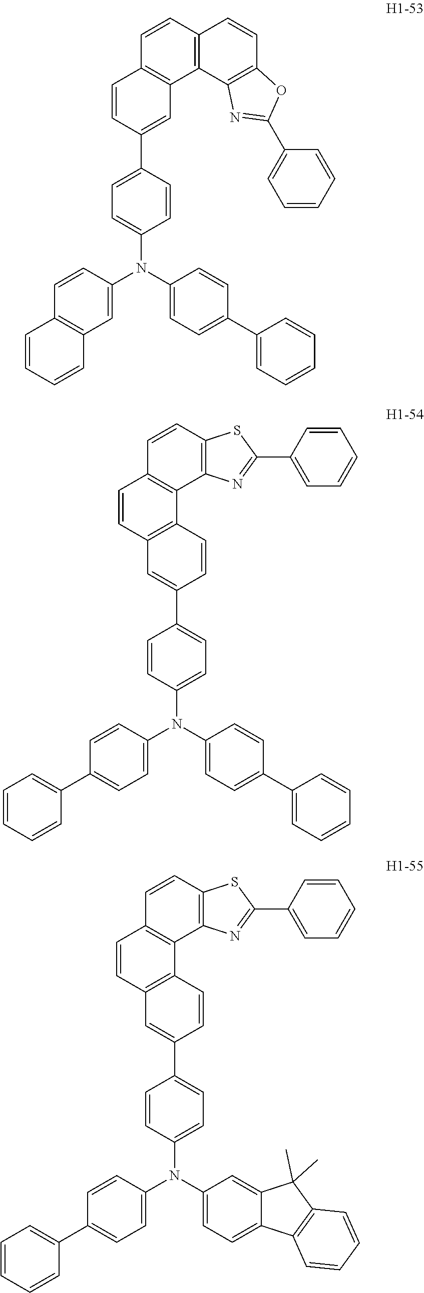

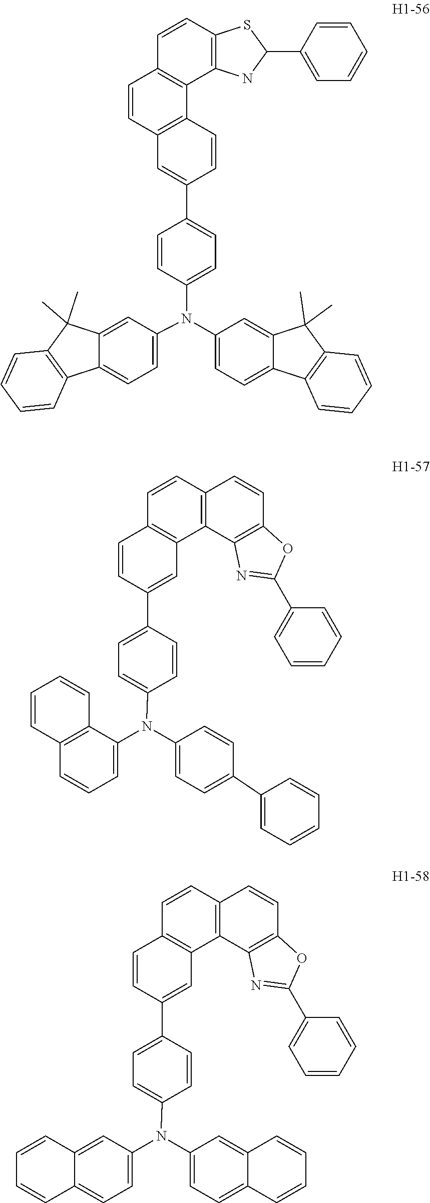

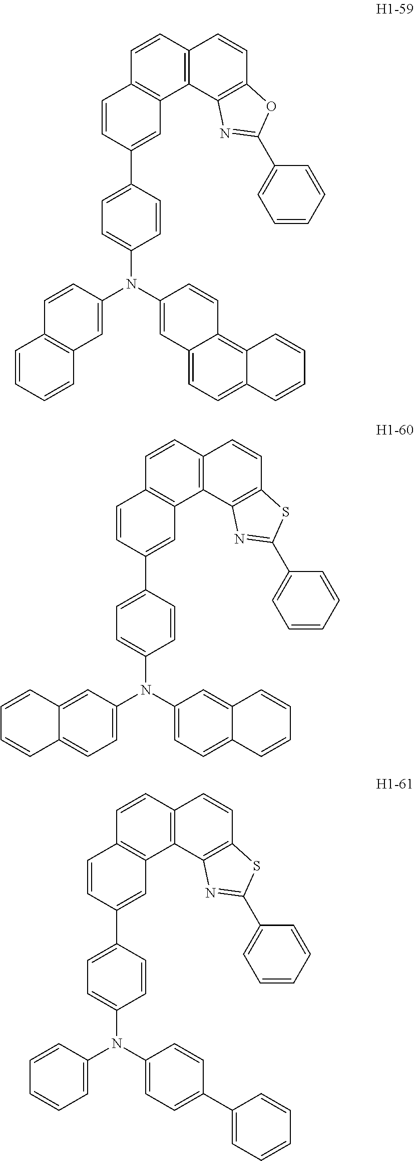

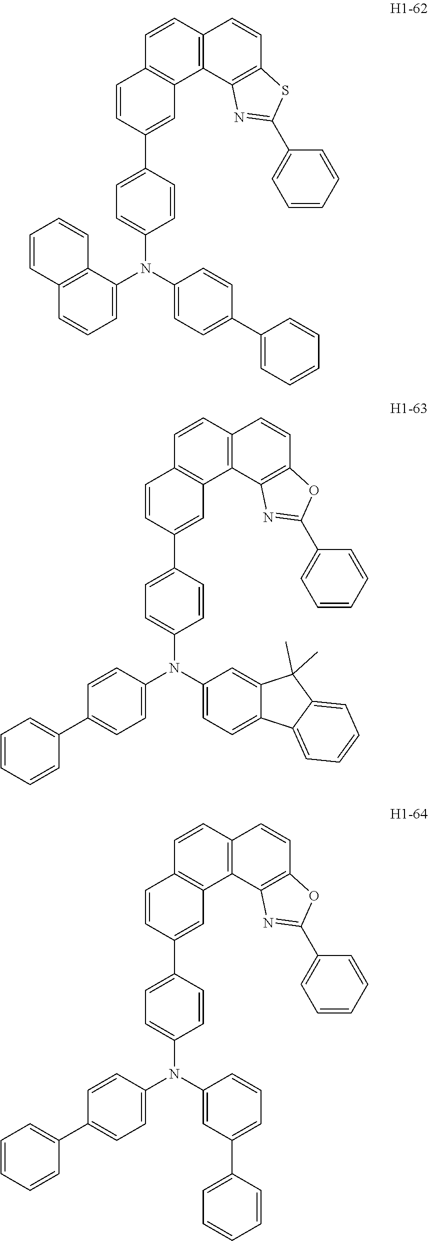

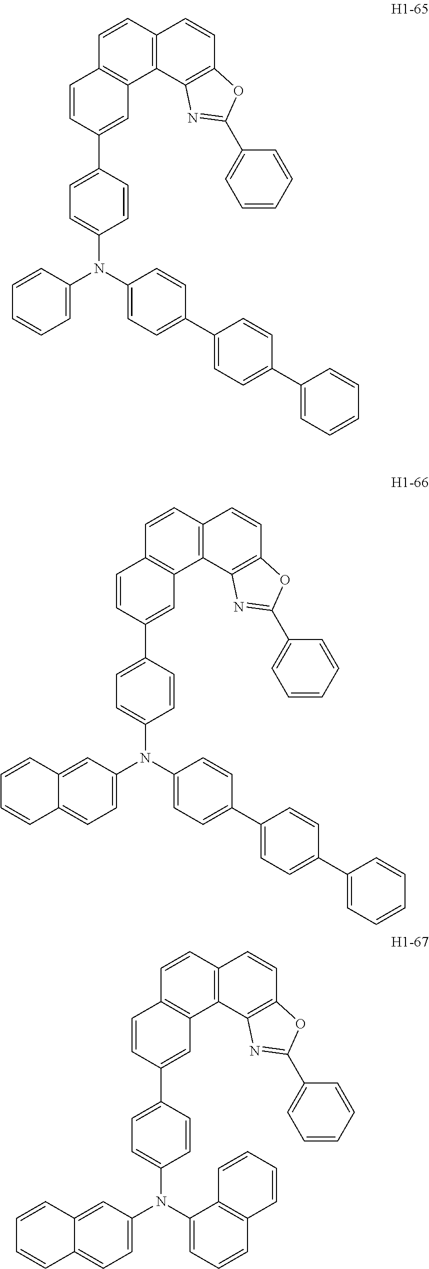 [SMILES: c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1]

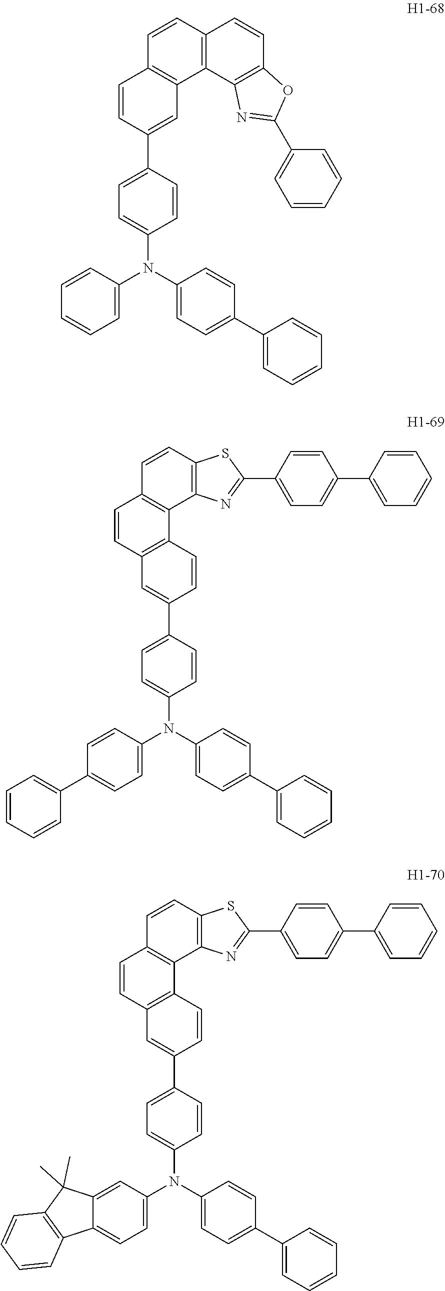 [SMILES: c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21]

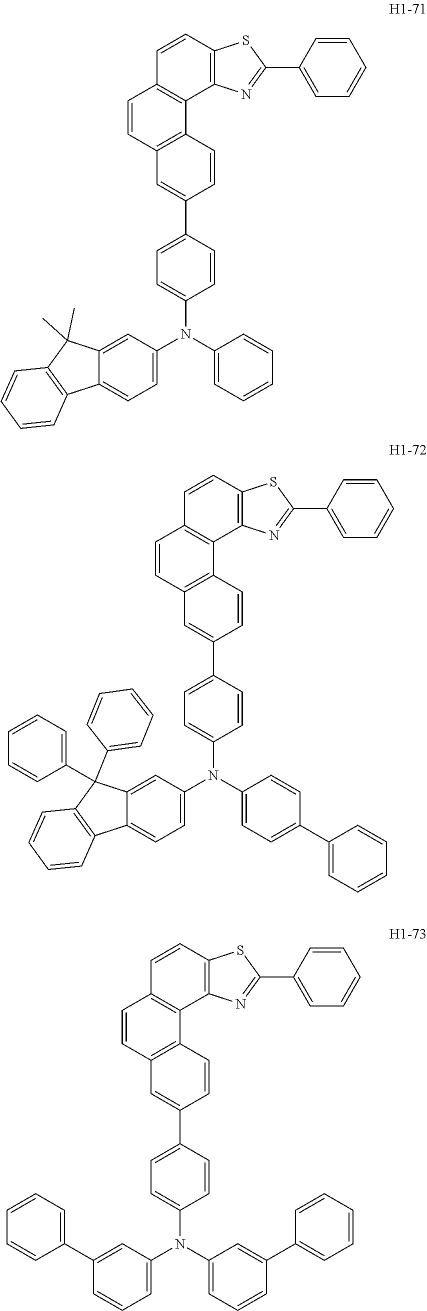 [SMILES: CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1]

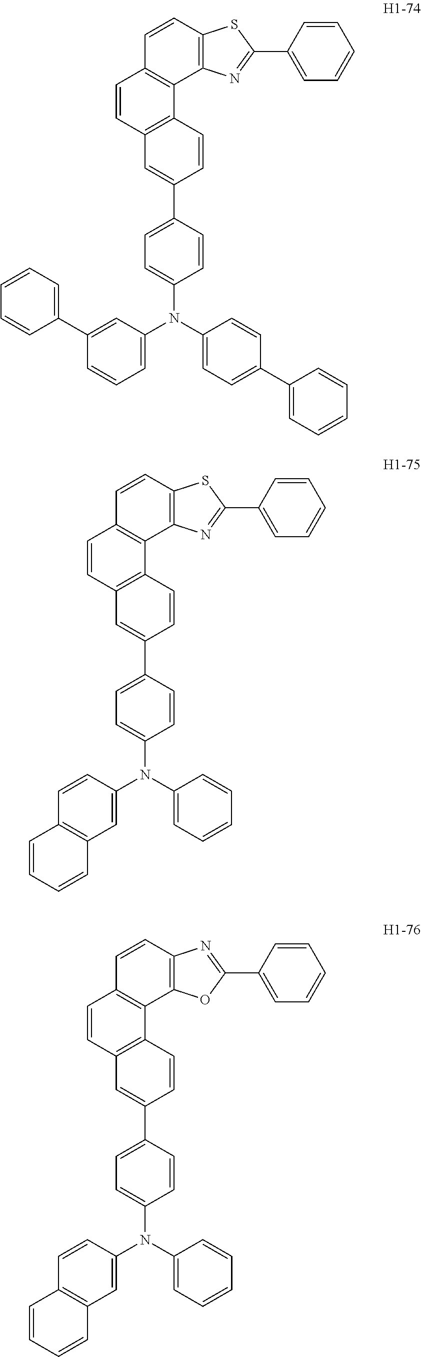

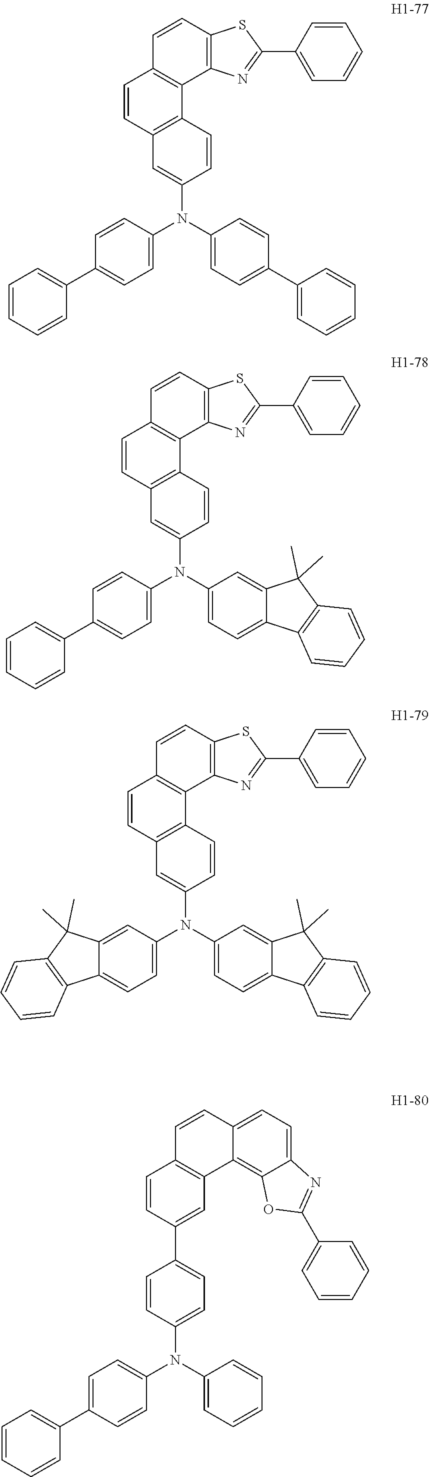

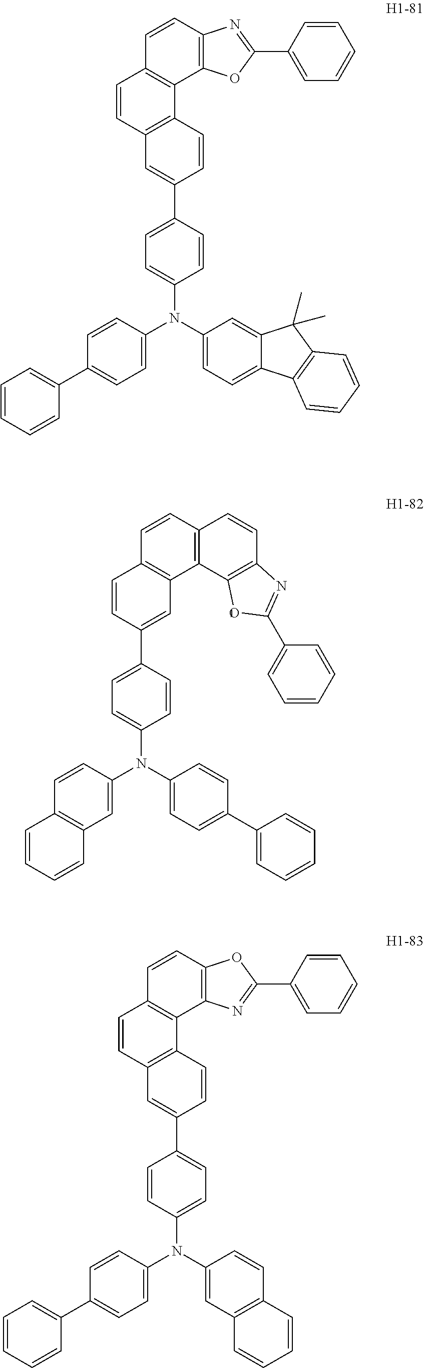

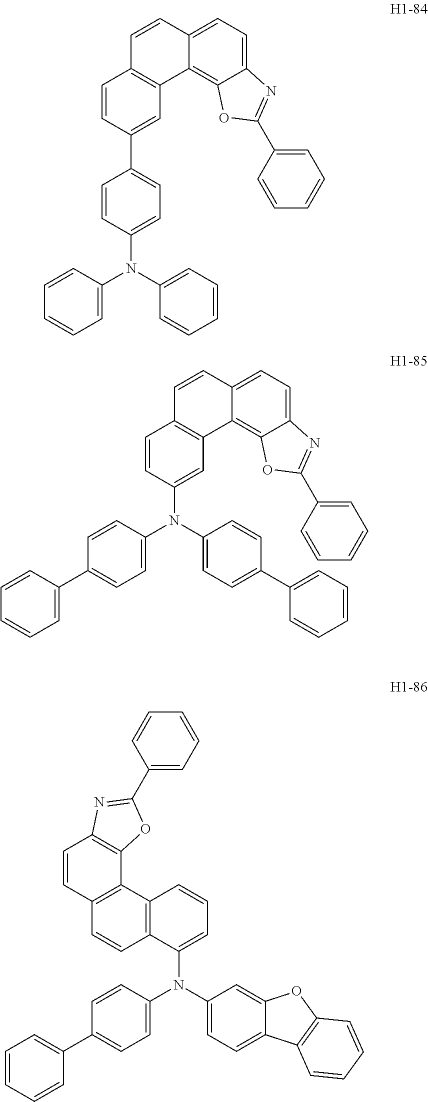

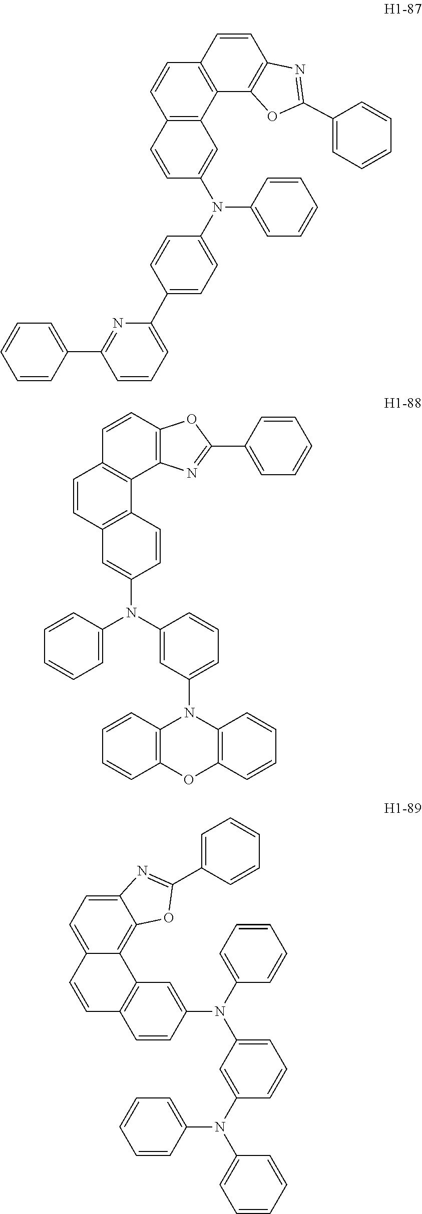

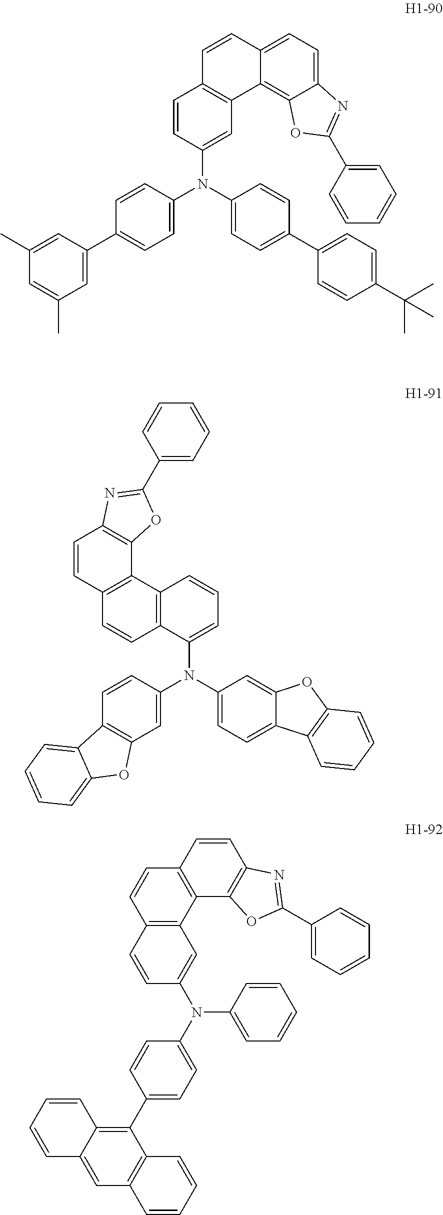 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1]

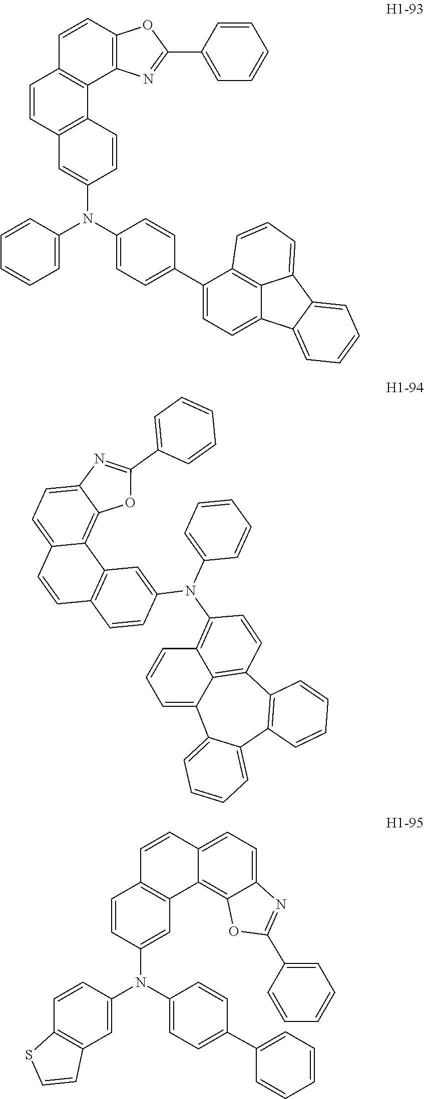

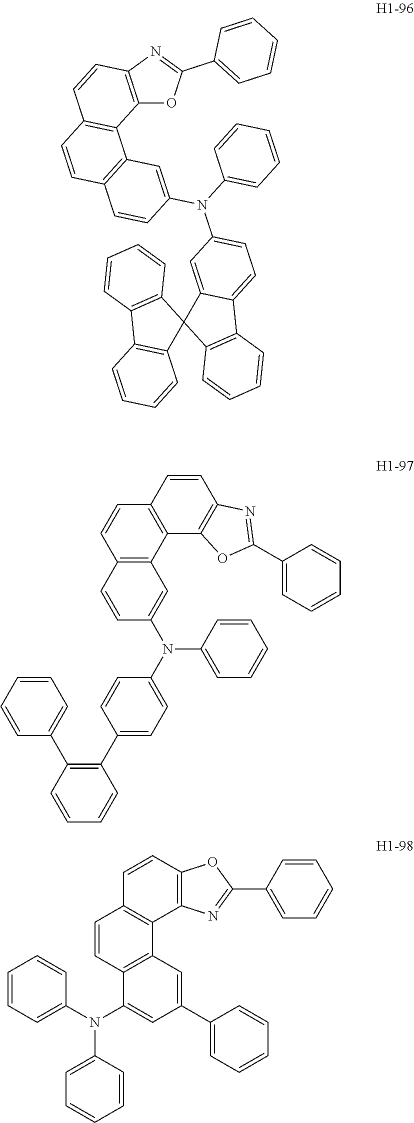 [SMILES: c1ccc(-c2nc3ccc4ccc5ccc(N(c6ccccc6)c6ccc7c(c6)C6(c8ccccc8-c8ccccc86)c6ccccc6-7)cc5c4c3o2)cc1 ; c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1]

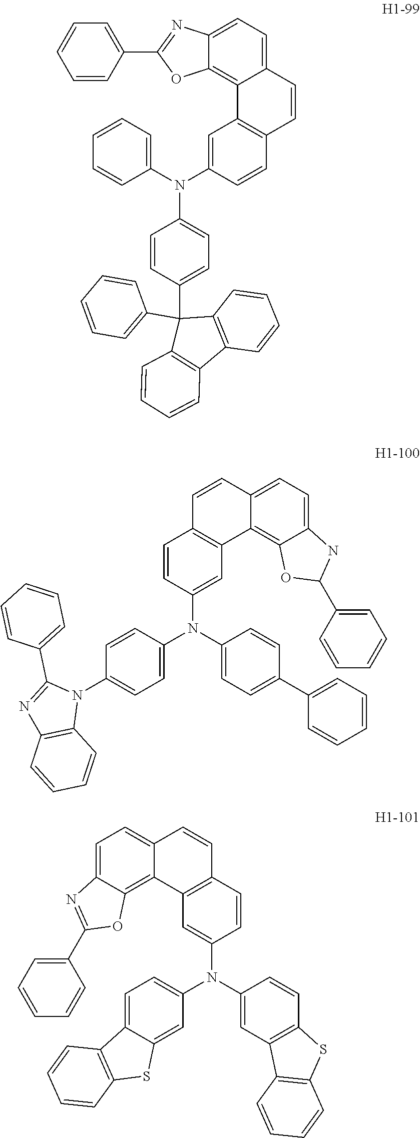 [SMILES: c1ccc(-c2nc3ccc4ccc5ccc(N(c6ccccc6)c6ccc(C7(c8ccccc8)c8ccccc8-c8ccccc87)cc6)cc5c4c3o2)cc1 ; c1ccc(-c2ccc(N(c3ccc(-n4c(-c5ccccc5)nc5ccccc54)cc3)c3ccc4ccc5ccc6c(c5c4c3)OC(c3ccccc3)N6)cc2)cc1 ; c1ccc(-c2nc3ccc4ccc5ccc(N(c6ccc7c(c6)sc6ccccc67)c6ccc7sc8ccccc8c7c6)cc5c4c3o2)cc1]

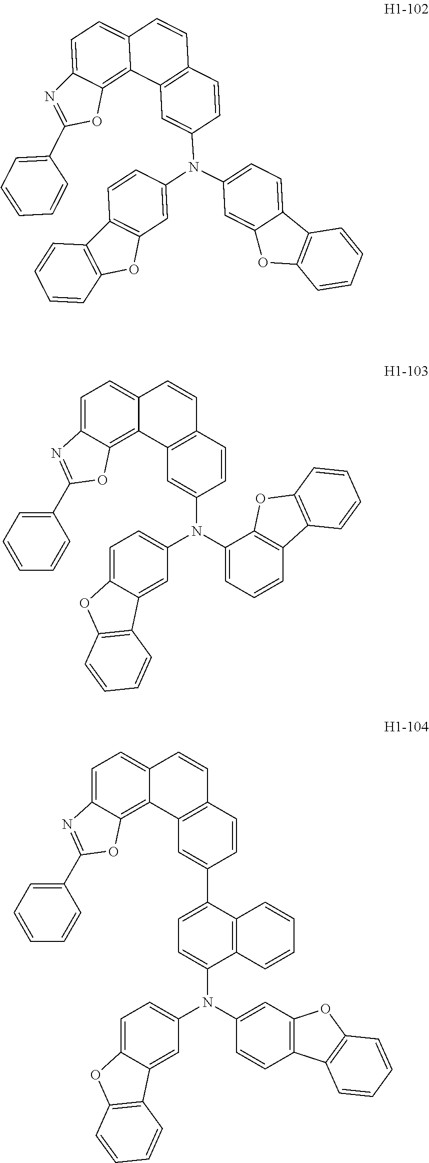 [SMILES: c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1]

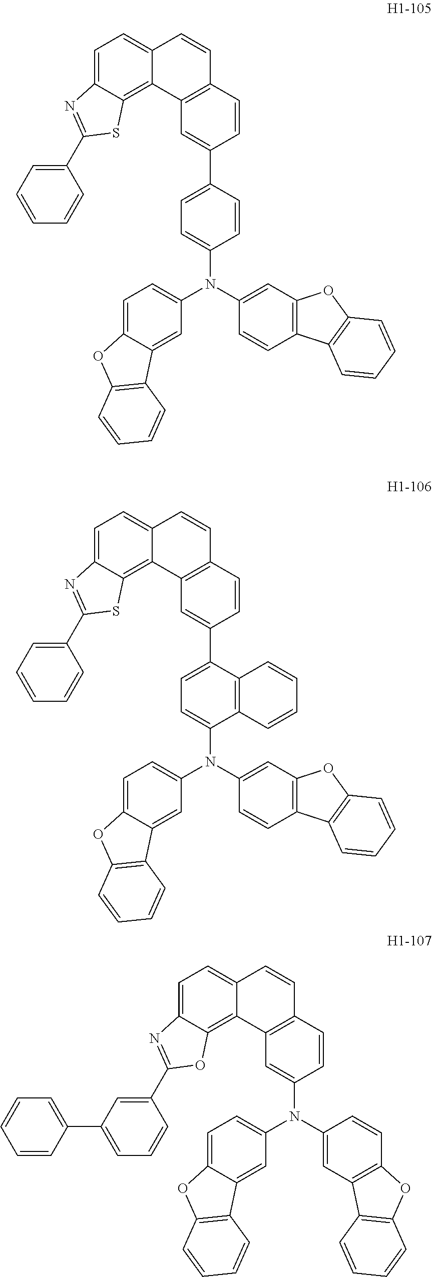

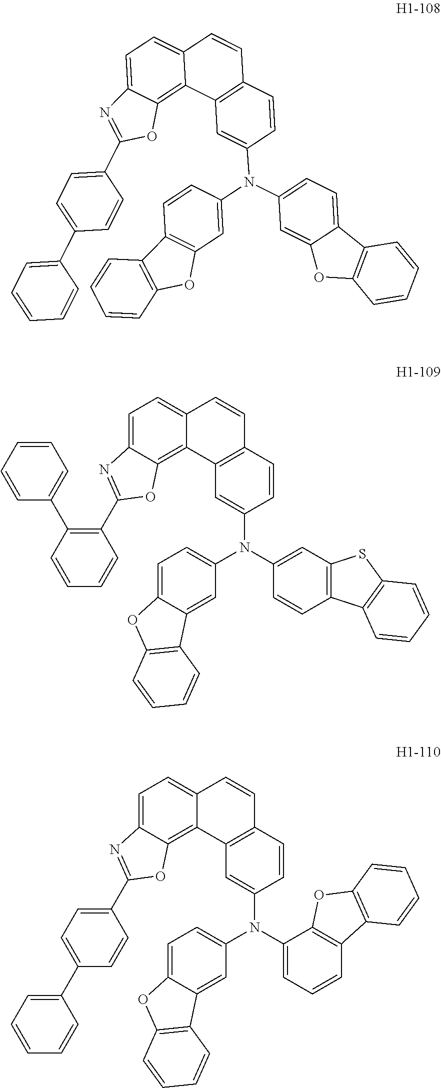 [SMILES: c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1]

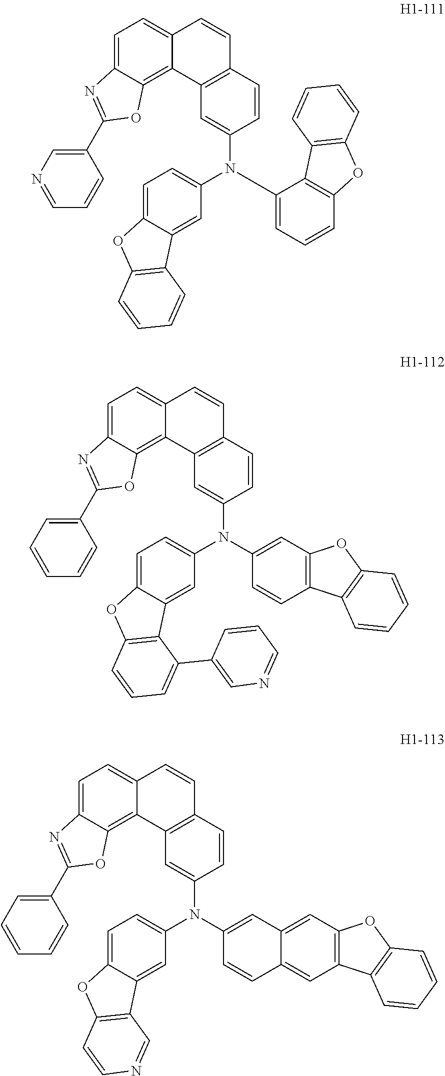

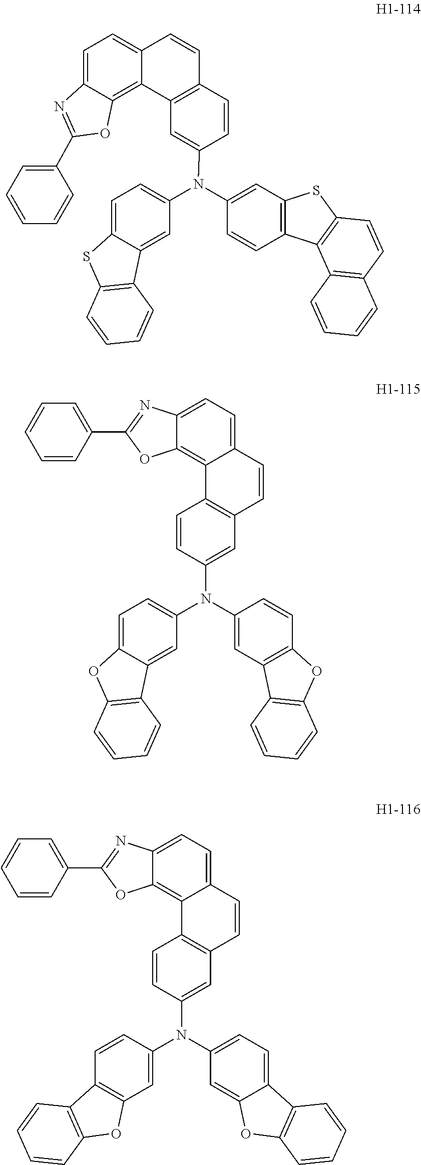 [SMILES: c1ccc(-c2nc3ccc4ccc5ccc(N(c6ccc7c(c6)sc6ccc8ccccc8c67)c6ccc7sc8ccccc8c7c6)cc5c4c3o2)cc1 ; c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1]

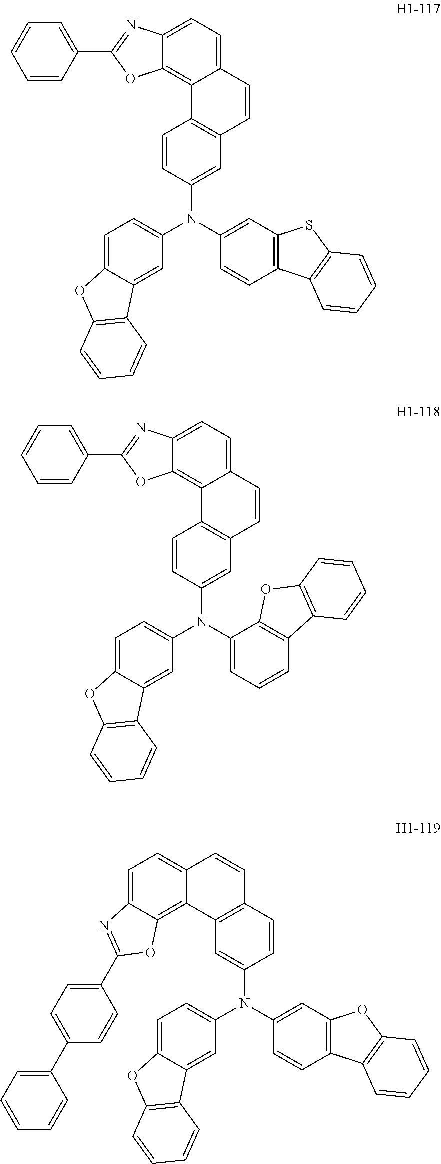 [SMILES: c1ccc(-c2nc3ccc4ccc5cc(N(c6ccc7c(c6)sc6ccccc67)c6ccc7oc8ccccc8c7c6)ccc5c4c3o2)cc1 ; c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1]

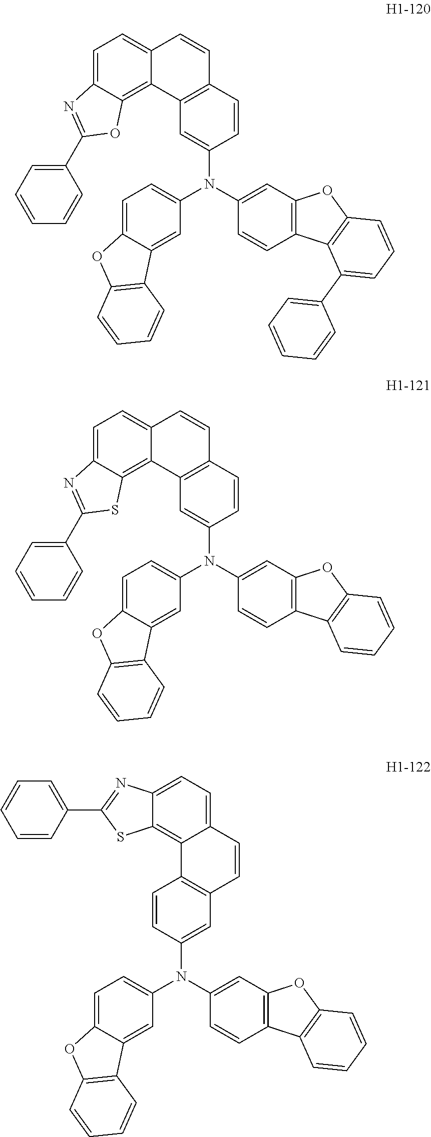 [SMILES: c1ccc(-c2nc3ccc4ccc5ccc(N(c6ccc7c(c6)oc6cccc(-c8ccccc8)c67)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1]

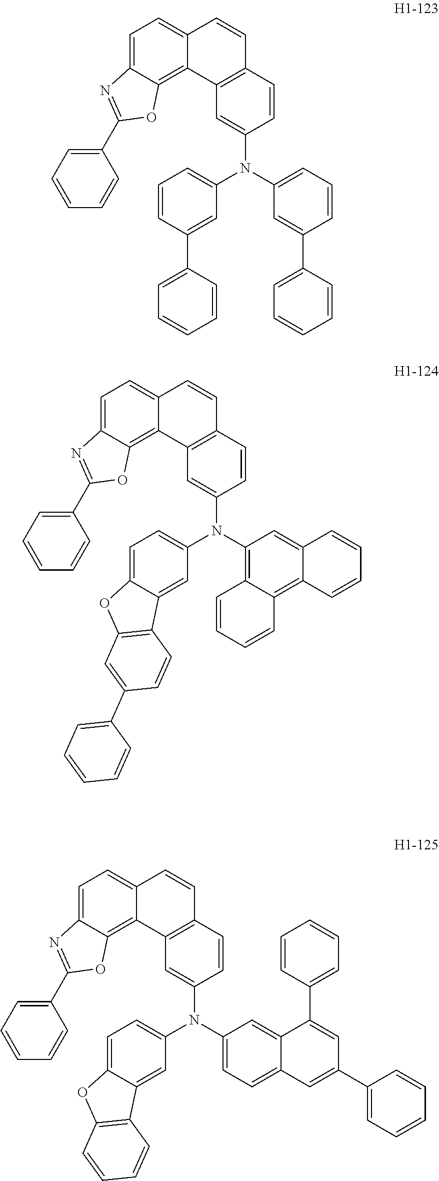

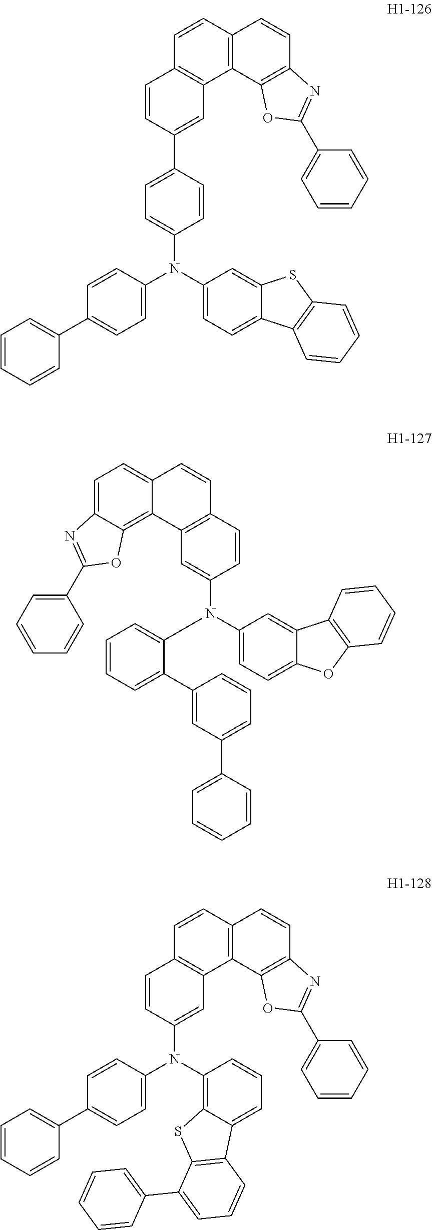 [SMILES: c1ccc(-c2ccc(N(c3ccc(-c4ccc5ccc6ccc7nc(-c8ccccc8)oc7c6c5c4)cc3)c3ccc4c(c3)sc3ccccc34)cc2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3c(-c5ccccc5)cccc34)cc2)cc1]

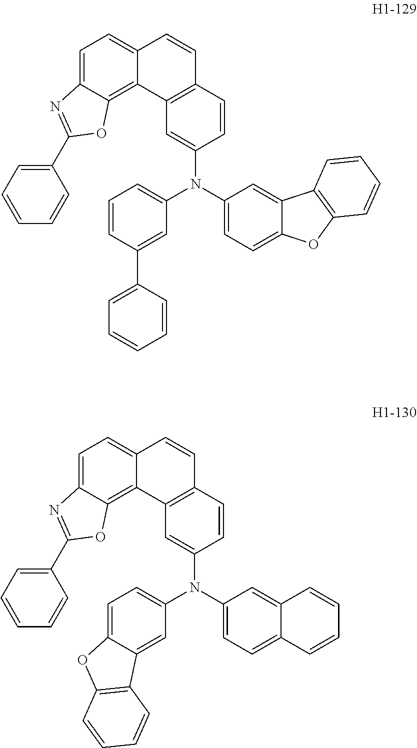 [SMILES: c1ccc(-c2cccc(N(c3ccc4oc5ccccc5c4c3)c3ccc4ccc5ccc6nc(-c7ccccc7)oc6c5c4c3)c2)cc1 ; c1ccc(-c2nc3ccc4ccc5ccc(N(c6ccc7ccccc7c6)c6ccc7oc8ccccc8c7c6)cc5c4c3o2)cc1]

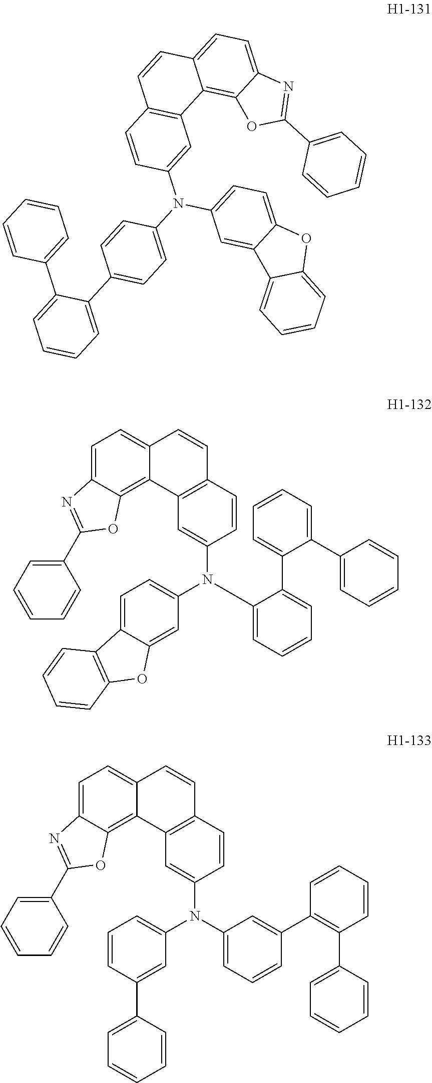 [SMILES: c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1 ; c1ccc(-c2cccc(N(c3cccc(-c4ccccc4-c4ccccc4)c3)c3ccc4ccc5ccc6nc(-c7ccccc7)oc6c5c4c3)c2)cc1]

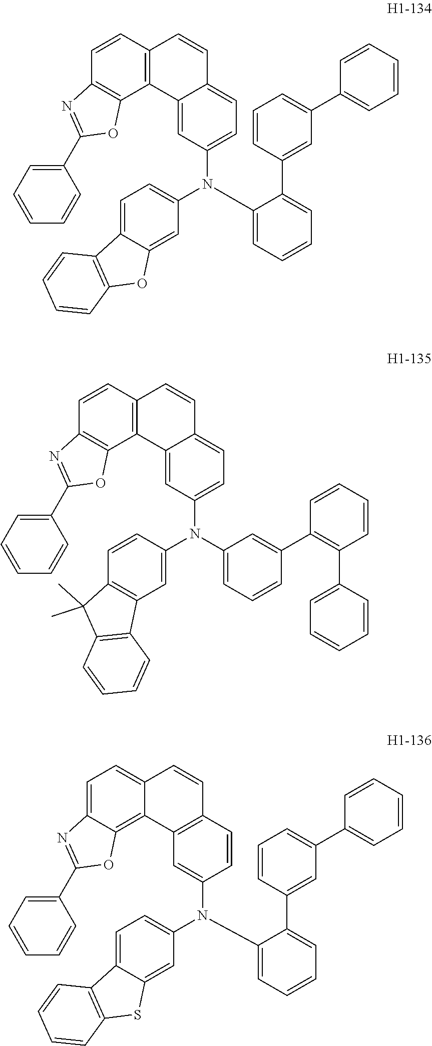 [SMILES: c1ccc(-c2cccc(-c3ccccc3N(c3ccc4c(c3)oc3ccccc34)c3ccc4ccc5ccc6nc(-c7ccccc7)oc6c5c4c3)c2)cc1 ; CC1(C)c2ccccc2-c2cc(N(c3cccc(-c4ccccc4-c4ccccc4)c3)c3ccc4ccc5ccc6nc(-c7ccccc7)oc6c5c4c3)ccc21 ; c1ccc(-c2cccc(-c3ccccc3N(c3ccc4c(c3)sc3ccccc34)c3ccc4ccc5ccc6nc(-c7ccccc7)oc6c5c4c3)c2)cc1]

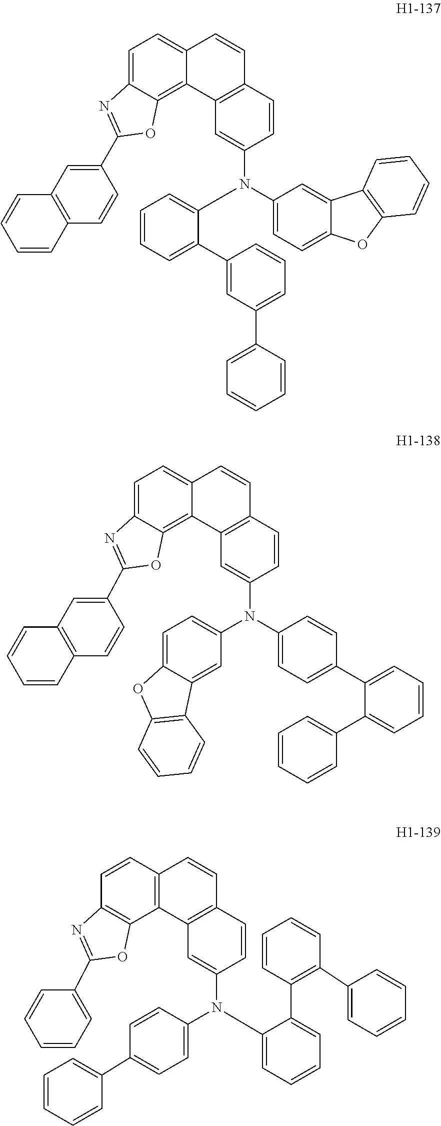 [SMILES: c1ccc(-c2cccc(-c3ccccc3N(c3ccc4oc5ccccc5c4c3)c3ccc4ccc5ccc6nc(-c7ccc8ccccc8c7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1 ; c1ccc(-c2ccc(N(c3ccc4ccc5ccc6nc(-c7ccccc7)oc6c5c4c3)c3ccccc3-c3ccccc3-c3ccccc3)cc2)cc1]

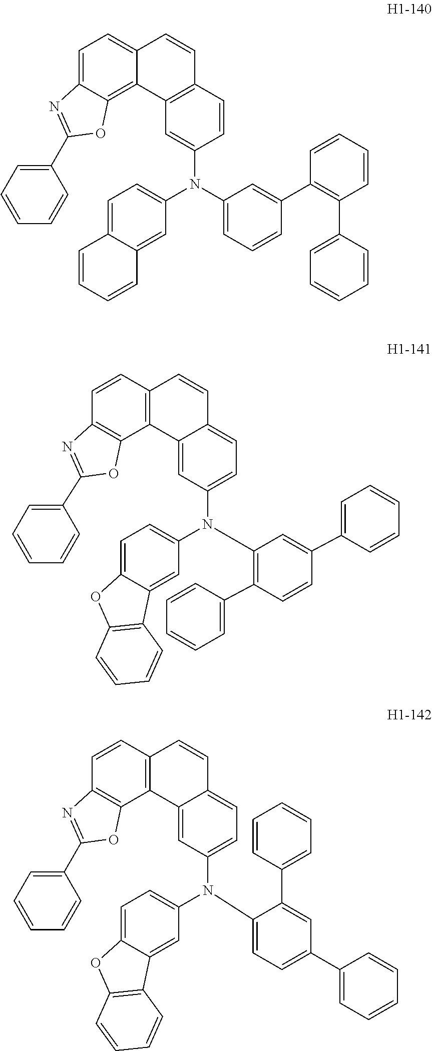 [SMILES: c1ccc(-c2nc3ccc4ccc5ccc(N(c6cccc(-c7ccccc7-c7ccccc7)c6)c6ccc7ccccc7c6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1]

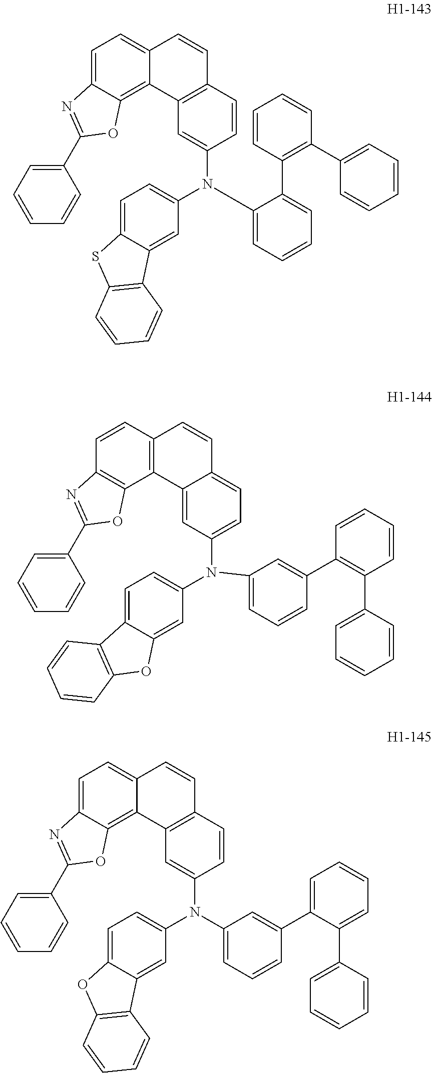 [SMILES: c1ccc(-c2nc3ccc4ccc5ccc(N(c6ccc7sc8ccccc8c7c6)c6ccccc6-c6ccccc6-c6ccccc6)cc5c4c3o2)cc1 ; c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1]

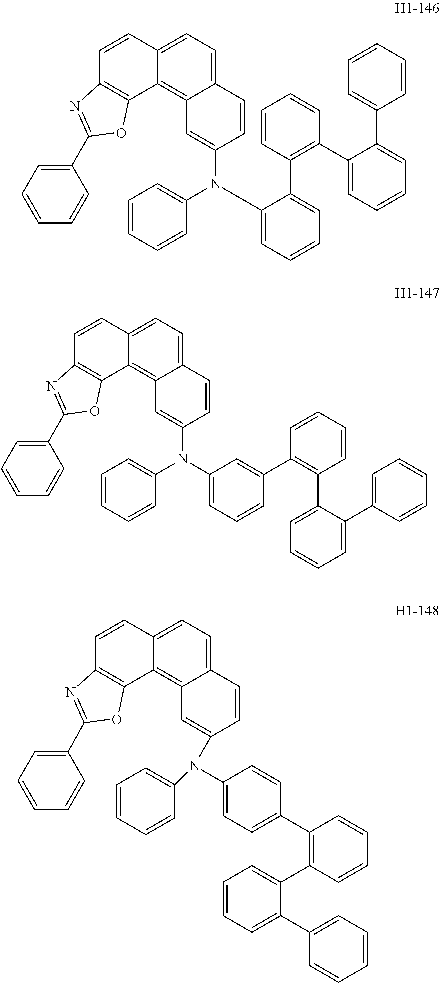 [SMILES: c1ccc(-c2nc3ccc4ccc5ccc(N(c6ccccc6)c6ccccc6-c6ccccc6-c6ccccc6-c6ccccc6)cc5c4c3o2)cc1 ; c1ccc(-c2nc3ccc4ccc5ccc(N(c6ccccc6)c6cccc(-c7ccccc7-c7ccccc7-c7ccccc7)c6)cc5c4c3o2)cc1 ; c1ccc(-c2nc3ccc4ccc5ccc(N(c6ccccc6)c6ccc(-c7ccccc7-c7ccccc7-c7ccccc7)cc6)cc5c4c3o2)cc1]

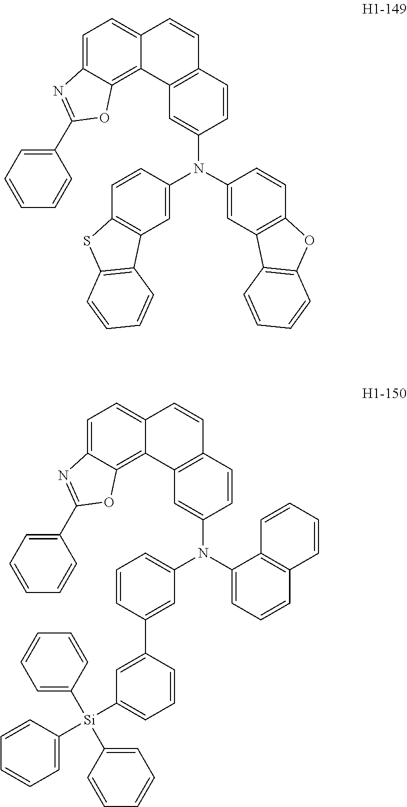 [SMILES: c1ccc(-c2nc3ccc4ccc5ccc(N(c6ccc7oc8ccccc8c7c6)c6ccc7sc8ccccc8c7c6)cc5c4c3o2)cc1 ; c1ccc(-c2nc3ccc4ccc5ccc(N(c6cccc(-c7cccc([Si](c8ccccc8)(c8ccccc8)c8ccccc8)c7)c6)c6cccc7ccccc67)cc5c4c3o2)cc1]

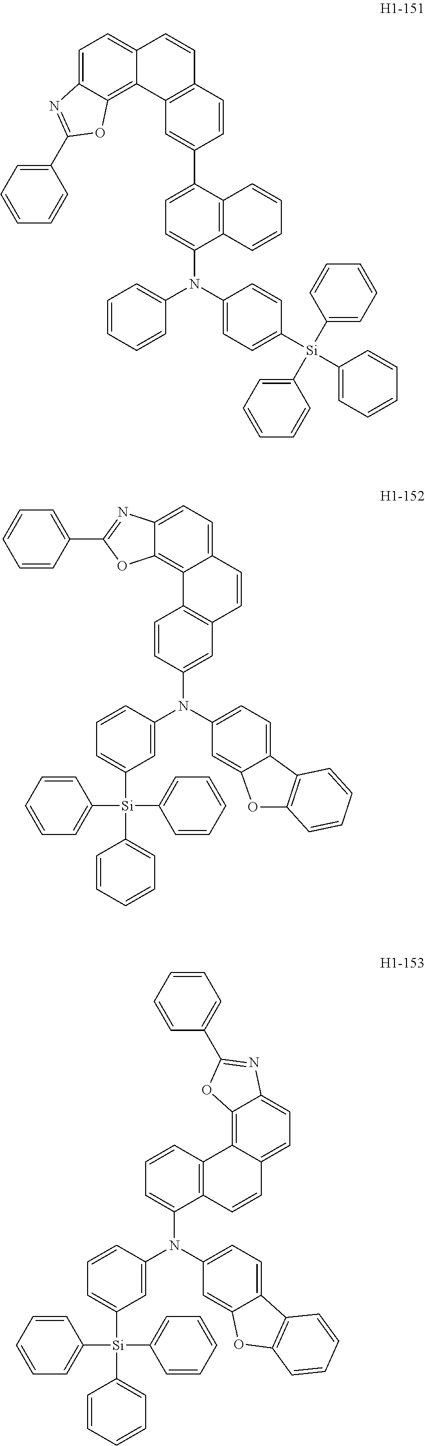 [SMILES: c1ccc(-c2nc3ccc4ccc5ccc(-c6ccc(N(c7ccccc7)c7ccc([Si](c8ccccc8)(c8ccccc8)c8ccccc8)cc7)c7ccccc67)cc5c4c3o2)cc1 ; c1ccc(-c2nc3ccc4ccc5cc(N(c6cccc([Si](c7ccccc7)(c7ccccc7)c7ccccc7)c6)c6ccc7c(c6)oc6ccccc67)ccc5c4c3o2)cc1 ; c1ccc(-c2nc3ccc4ccc5c(N(c6cccc([Si](c7ccccc7)(c7ccccc7)c7ccccc7)c6)c6ccc7c(c6)oc6ccccc67)cccc5c4c3o2)cc1]

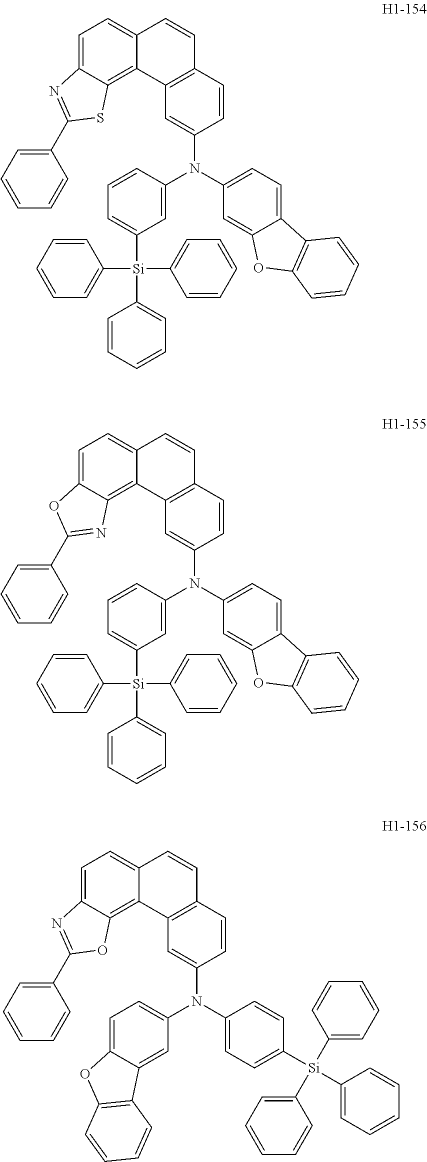 [SMILES: c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3s2)cc1 ; c1ccc(-c2nc3c(ccc4ccc5ccc(N(c6cccc([Si](c7ccccc7)(c7ccccc7)c7ccccc7)c6)c6ccc7c(c6)oc6ccccc67)cc5c43)o2)cc1 ; c1ccc(-c2nc3ccc4ccc5ccc(N(c6ccc([Si](c7ccccc7)(c7ccccc7)c7ccccc7)cc6)c6ccc7oc8ccccc8c7c6)cc5c4c3o2)cc1]

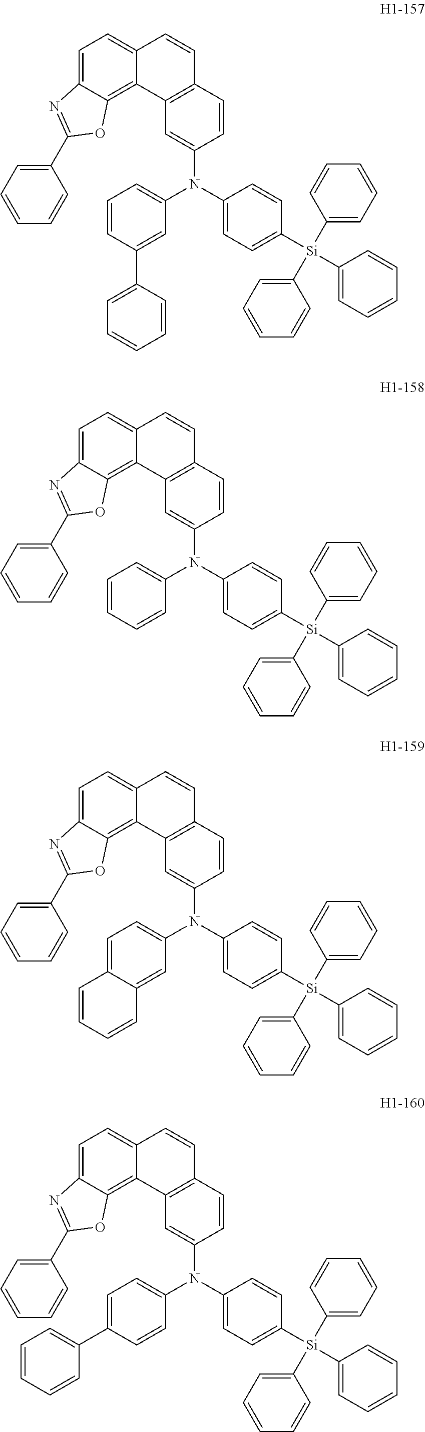 [SMILES: c1ccc(-c2cccc(N(c3ccc([Si](c4ccccc4)(c4ccccc4)c4ccccc4)cc3)c3ccc4ccc5ccc6nc(-c7ccccc7)oc6c5c4c3)c2)cc1 ; c1ccc(-c2nc3ccc4ccc5ccc(N(c6ccccc6)c6ccc([Si](c7ccccc7)(c7ccccc7)c7ccccc7)cc6)cc5c4c3o2)cc1 ; c1ccc(-c2nc3ccc4ccc5ccc(N(c6ccc([Si](c7ccccc7)(c7ccccc7)c7ccccc7)cc6)c6ccc7ccccc7c6)cc5c4c3o2)cc1 ; c1ccc(-c2ccc(N(c3ccc([Si](c4ccccc4)(c4ccccc4)c4ccccc4)cc3)c3ccc4ccc5ccc6nc(-c7ccccc7)oc6c5c4c3)cc2)cc1]

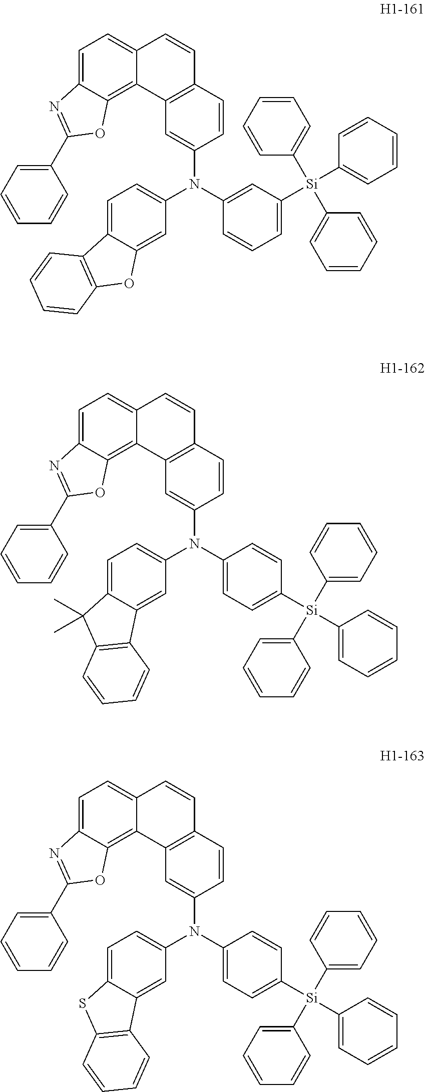 [SMILES: c1ccc(-c2nc3ccc4ccc5ccc(N(c6cccc([Si](c7ccccc7)(c7ccccc7)c7ccccc7)c6)c6ccc7c(c6)oc6ccccc67)cc5c4c3o2)cc1 ; CC1(C)c2ccccc2-c2cc(N(c3ccc([Si](c4ccccc4)(c4ccccc4)c4ccccc4)cc3)c3ccc4ccc5ccc6nc(-c7ccccc7)oc6c5c4c3)ccc21 ; c1ccc(-c2nc3ccc4ccc5ccc(N(c6ccc([Si](c7ccccc7)(c7ccccc7)c7ccccc7)cc6)c6ccc7sc8ccccc8c7c6)cc5c4c3o2)cc1]

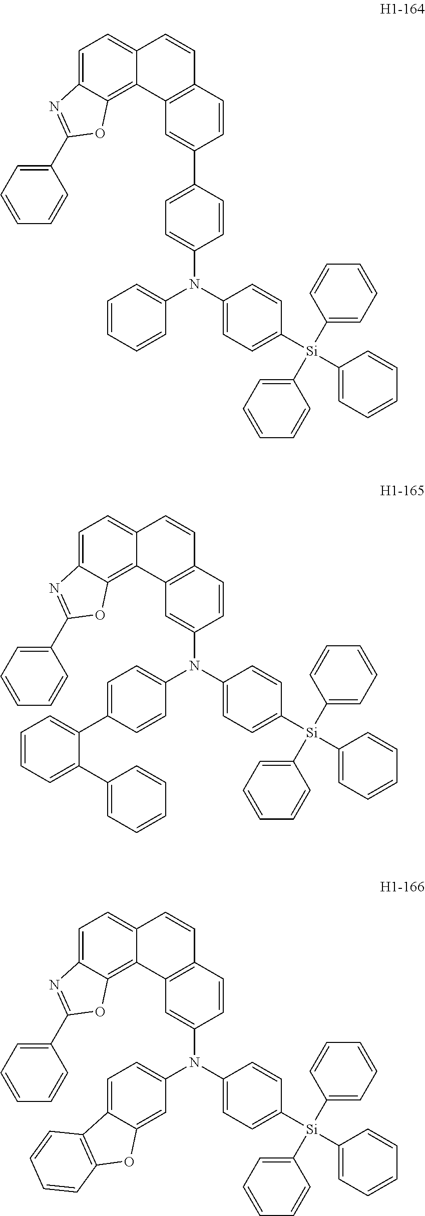 [SMILES: c1ccc(-c2nc3ccc4ccc5ccc(-c6ccc(N(c7ccccc7)c7ccc([Si](c8ccccc8)(c8ccccc8)c8ccccc8)cc7)cc6)cc5c4c3o2)cc1 ; c1ccc(-c2nc3ccc4ccc5ccc(N(c6ccc(-c7ccccc7-c7ccccc7)cc6)c6ccc([Si](c7ccccc7)(c7ccccc7)c7ccccc7)cc6)cc5c4c3o2)cc1 ; c1ccc(-c2nc3ccc4ccc5ccc(N(c6ccc([Si](c7ccccc7)(c7ccccc7)c7ccccc7)cc6)c6ccc7c(c6)oc6ccccc67)cc5c4c3o2)cc1]

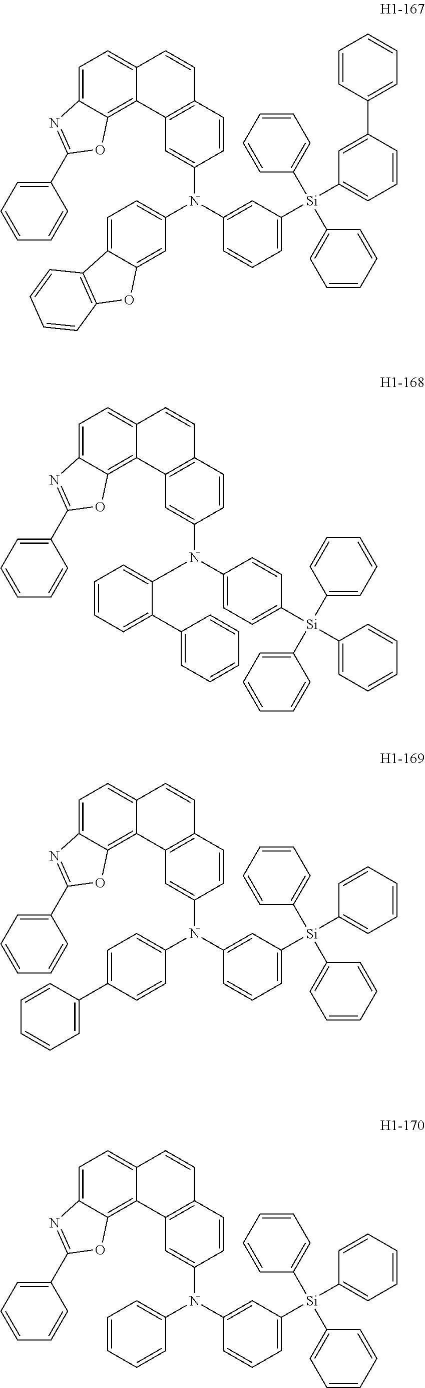 [SMILES: c1ccc(-c2cccc([Si](c3ccccc3)(c3ccccc3)c3cccc(N(c4ccc5c(c4)oc4ccccc45)c4ccc5ccc6ccc7nc(-c8ccccc8)oc7c6c5c4)c3)c2)cc1 ; c1ccc(-c2nc3ccc4ccc5ccc(N(c6ccc([Si](c7ccccc7)(c7ccccc7)c7ccccc7)cc6)c6ccccc6-c6ccccc6)cc5c4c3o2)cc1 ; c1ccc(-c2ccc(N(c3cccc([Si](c4ccccc4)(c4ccccc4)c4ccccc4)c3)c3ccc4ccc5ccc6nc(-c7ccccc7)oc6c5c4c3)cc2)cc1 ; c1ccc(-c2nc3ccc4ccc5ccc(N(c6ccccc6)c6cccc([Si](c7ccccc7)(c7ccccc7)c7ccccc7)c6)cc5c4c3o2)cc1]

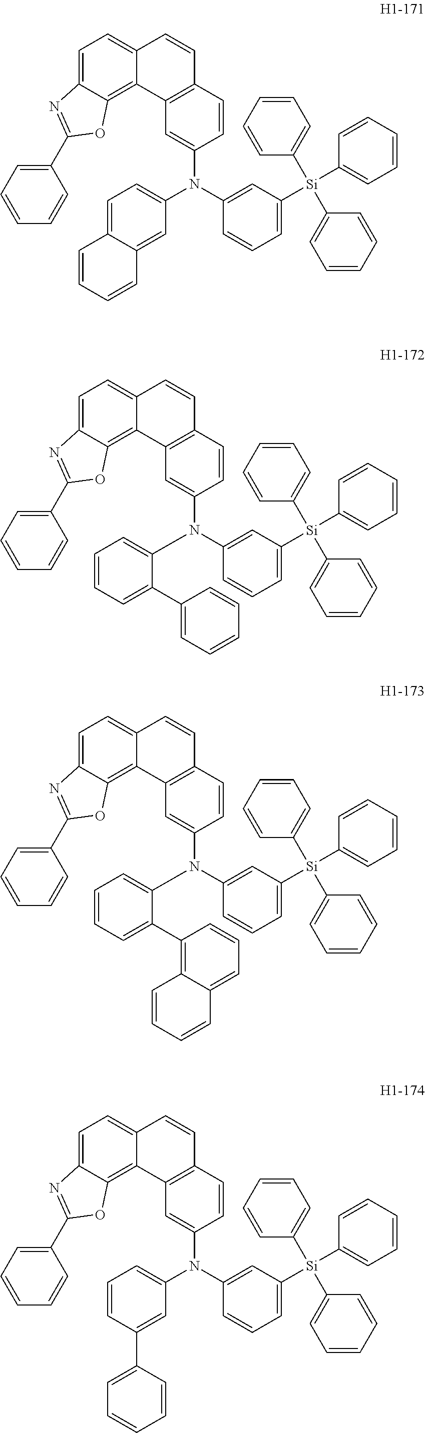 [SMILES: c1ccc(-c2nc3ccc4ccc5ccc(N(c6cccc([Si](c7ccccc7)(c7ccccc7)c7ccccc7)c6)c6ccc7ccccc7c6)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6ccccc6)cc5c4c3o2)cc1 ; c1ccc(-c2nc3ccc4ccc5ccc(N(c6cccc([Si](c7ccccc7)(c7ccccc7)c7ccccc7)c6)c6ccccc6-c6cccc7ccccc67)cc5c4c3o2)cc1 ; c1ccc(-c2cccc(N(c3cccc([Si](c4ccccc4)(c4ccccc4)c4ccccc4)c3)c3ccc4ccc5ccc6nc(-c7ccccc7)oc6c5c4c3)c2)cc1]

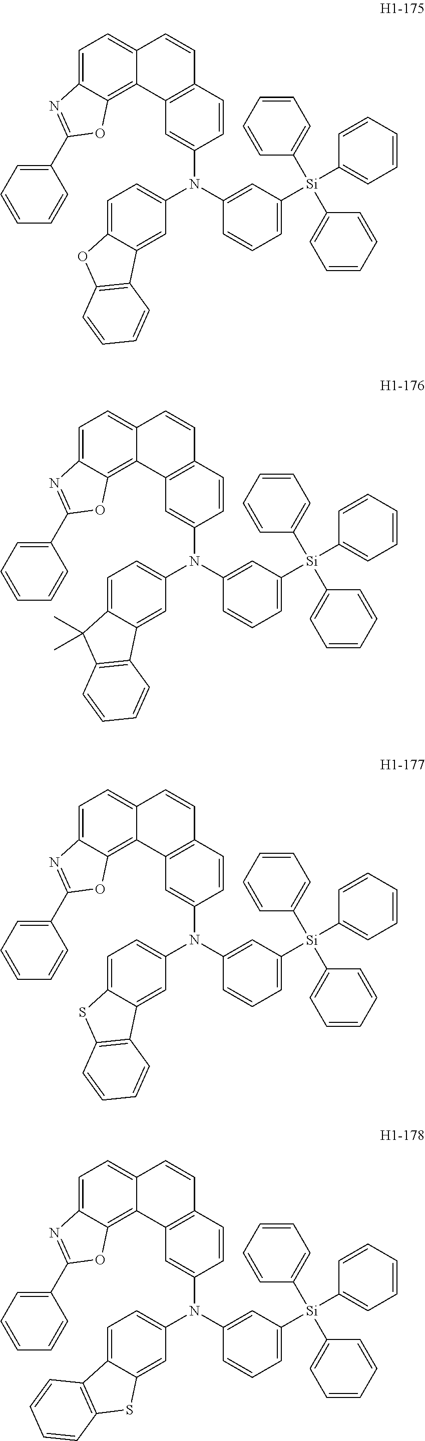 [SMILES: c1ccc(-c2nc3ccc4ccc5ccc(N(c6cccc([Si](c7ccccc7)(c7ccccc7)c7ccccc7)c6)c6ccc7oc8ccccc8c7c6)cc5c4c3o2)cc1 ; CC1(C)c2ccccc2-c2cc(N(c3cccc([Si](c4ccccc4)(c4ccccc4)c4ccccc4)c3)c3ccc4ccc5ccc6nc(-c7ccccc7)oc6c5c4c3)ccc21 ; c1ccc(-c2nc3ccc4ccc5ccc(N(c6cccc([Si](c7ccccc7)(c7ccccc7)c7ccccc7)c6)c6ccc7sc8ccccc8c7c6)cc5c4c3o2)cc1 ; c1ccc(-c2nc3ccc4ccc5ccc(N(c6cccc([Si](c7ccccc7)(c7ccccc7)c7ccccc7)c6)c6ccc7c(c6)sc6ccccc67)cc5c4c3o2)cc1]

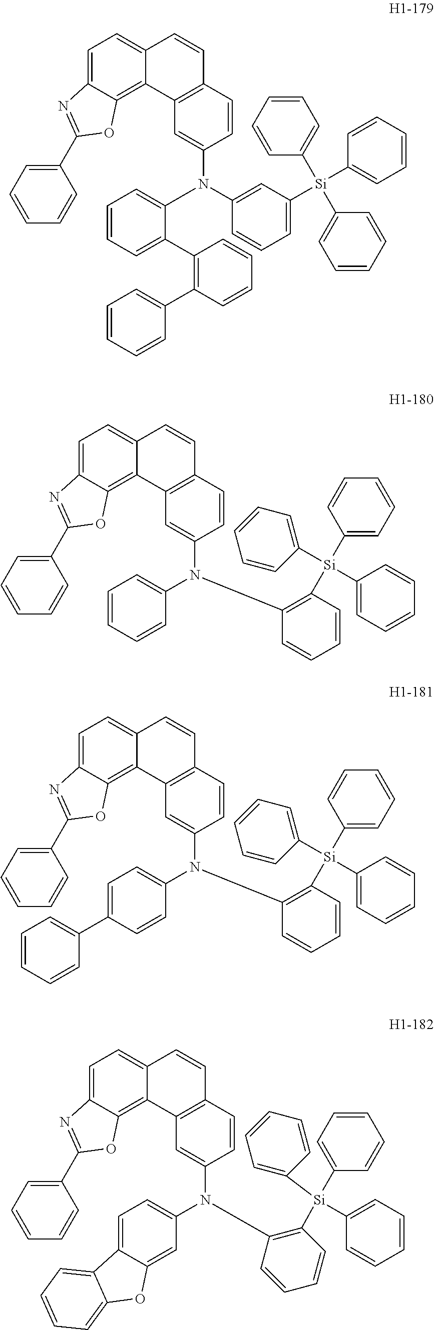 [SMILES: c1ccc(-c2nc3ccc4ccc5ccc(N(c6cccc([Si](c7ccccc7)(c7ccccc7)c7ccccc7)c6)c6ccccc6-c6ccccc6-c6ccccc6)cc5c4c3o2)cc1 ; c1ccc(-c2nc3ccc4ccc5ccc(N(c6ccccc6)c6ccccc6[Si](c6ccccc6)(c6ccccc6)c6ccccc6)cc5c4c3o2)cc1 ; c1ccc(-c2ccc(N(c3ccc4ccc5ccc6nc(-c7ccccc7)oc6c5c4c3)c3ccccc3[Si](c3ccccc3)(c3ccccc3)c3ccccc3)cc2)cc1 ; c1ccc(-c2nc3ccc4ccc5ccc(N(c6ccc7c(c6)oc6ccccc67)c6ccccc6[Si](c6ccccc6)(c6ccccc6)c6ccccc6)cc5c4c3o2)cc1]

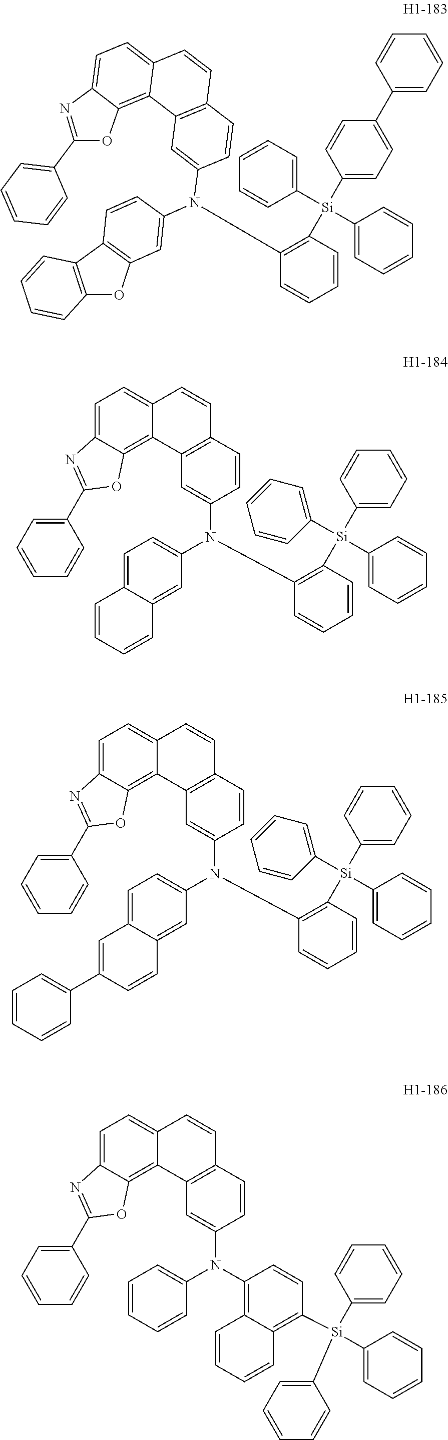 [SMILES: c1ccc(-c2ccc([Si](c3ccccc3)(c3ccccc3)c3ccccc3N(c3ccc4c(c3)oc3ccccc34)c3ccc4ccc5ccc6nc(-c7ccccc7)oc6c5c4c3)cc2)cc1 ; c1ccc(-c2nc3ccc4ccc5ccc(N(c6ccc7ccccc7c6)c6ccccc6[Si](c6ccccc6)(c6ccccc6)c6ccccc6)cc5c4c3o2)cc1 ; c1ccc(-c2ccc3cc(N(c4ccc5ccc6ccc7nc(-c8ccccc8)oc7c6c5c4)c4ccccc4[Si](c4ccccc4)(c4ccccc4)c4ccccc4)ccc3c2)cc1 ; c1ccc(-c2nc3ccc4ccc5ccc(N(c6ccccc6)c6ccc([Si](c7ccccc7)(c7ccccc7)c7ccccc7)c7ccccc67)cc5c4c3o2)cc1]

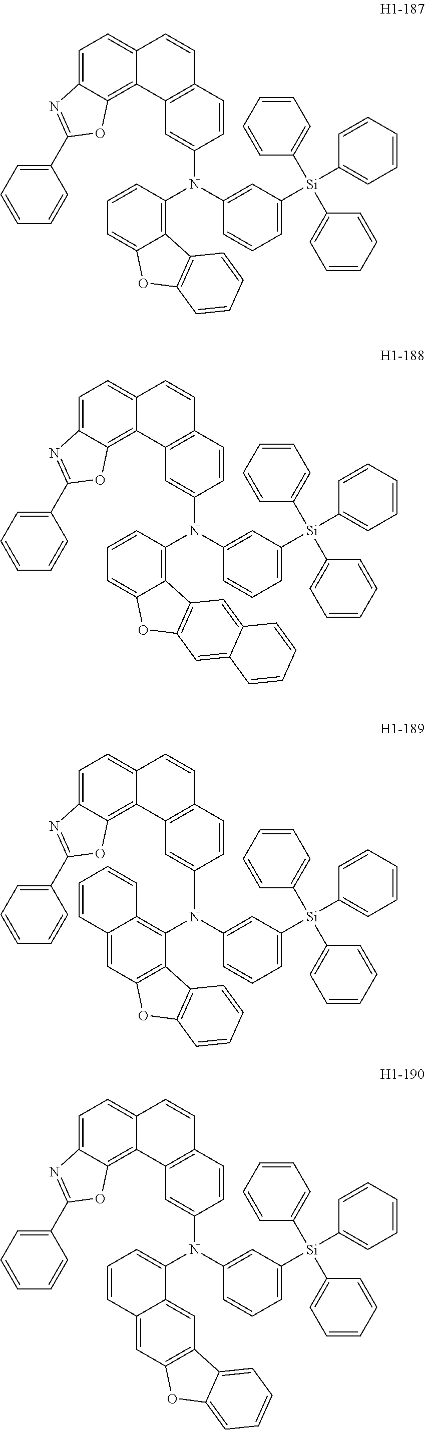 [SMILES: c1ccc(-c2nc3ccc4ccc5ccc(N(c6cccc([Si](c7ccccc7)(c7ccccc7)c7ccccc7)c6)c6cccc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6cccc7oc8cc9ccccc9cc8c67)cc5c4c3o2)cc1 ; c1ccc(-c2nc3ccc4ccc5ccc(N(c6cccc([Si](c7ccccc7)(c7ccccc7)c7ccccc7)c6)c6c7ccccc7cc7oc8ccccc8c67)cc5c4c3o2)cc1 ; c1ccc(-c2nc3ccc4ccc5ccc(N(c6cccc([Si](c7ccccc7)(c7ccccc7)c7ccccc7)c6)c6cccc7cc8oc9ccccc9c8cc67)cc5c4c3o2)cc1]

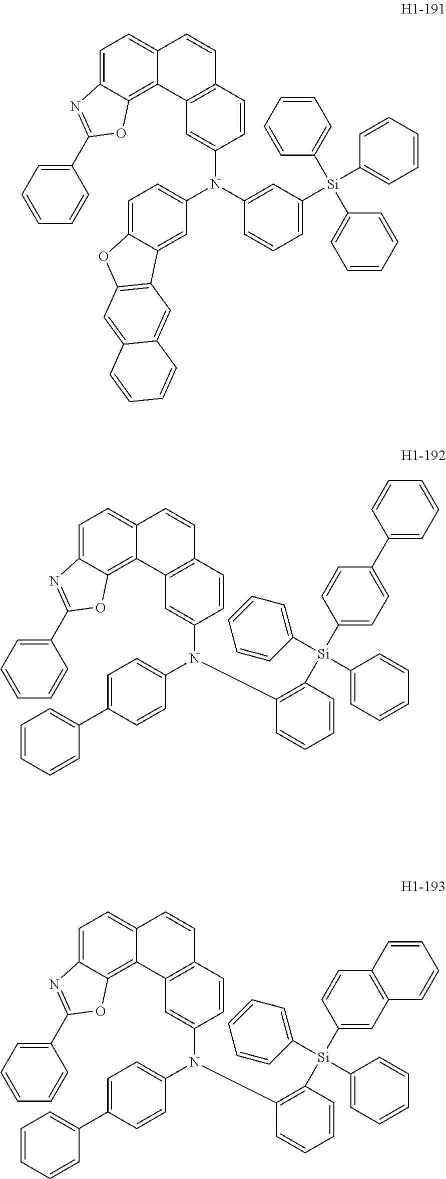 [SMILES: c1ccc(-c2nc3ccc4ccc5ccc(N(c6cccc([Si](c7ccccc7)(c7ccccc7)c7ccccc7)c6)c6ccc7oc8cc9ccccc9cc8c7c6)cc5c4c3o2)cc1 ; c1ccc(-c2ccc(N(c3ccc4ccc5ccc6nc(-c7ccccc7)oc6c5c4c3)c3ccccc3[Si](c3ccccc3)(c3ccccc3)c3ccc(-c4ccccc4)cc3)cc2)cc1 ; c1ccc(-c2ccc(N(c3ccc4ccc5ccc6nc(-c7ccccc7)oc6c5c4c3)c3ccccc3[Si](c3ccccc3)(c3ccccc3)c3ccc4ccccc4c3)cc2)cc1]

The host compound represented by Formula 1 according to the present disclosure can be manufactured by referring to a synthesis method known to those skilled in the art, for example, the synthesis method disclosed in Korean Patent Application Laid-open Nos. 2017-0022865 and 2018-0099487, etc.

The second host compound as another host material according to one embodiment may be represented by the following Formula 2.

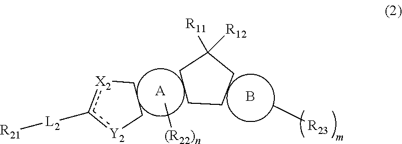

In Formula 2,
ring A and ring B each independently represent a substituted or unsubstituted (C₆-C₃₀)arene or a substituted or unsubstituted (3- to 30-membered)heteroarene;
R₁₁ and R₁₂ each independently represent a substituted or unsubstituted (C₁-C₃₀)alkyl, a substituted or unsubstituted (C₆-C₃₀)aryl, or a substituted or unsubstituted (3-to 30-membered)heteroaryl; or may be linked to each other to form a ring(s);
X₂ and Y₂ each independently represent —N=, —NR₂₄, —O—, or —S—; provided that any one of X₂ and Y₂ is —N=, and the other of X₂ and Y₂ is —NR₂₄—, —O—, or —S—;
R₂₁ to R₂₄ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and a (C₆-C₃₀) aromatic ring, or —L₃-HAr;
provided that at least one of R₂₁ to R₂₄ is —L₃-HAr;
L₂ and L₃ each independently represent a single bond, a substituted or unsubstituted (C₆-C₃₀)arylene, or a substituted or unsubstituted (3- to 30-membered)heteroarylene;
HAr represents a substituted or unsubstituted (3- to 30-membered)heteroaryl containing at least one nitrogen atom; and
n represents an integer of 1 to 4, m represents an integer of 1 to 8, and when n and m are an integer of 2 or more, each of R₂₂ and R₂₃ may be the same or different.

In one embodiment, ring A may be benzene ring or naphthalene ring.

In one embodiment, ring B may be benzene ring, naphthalene ring, or phenanthrene ring.

According to one embodiment, the host compound represented by Formula 2 may be represented by any one of the following Formulas 2-1 to 2-8.

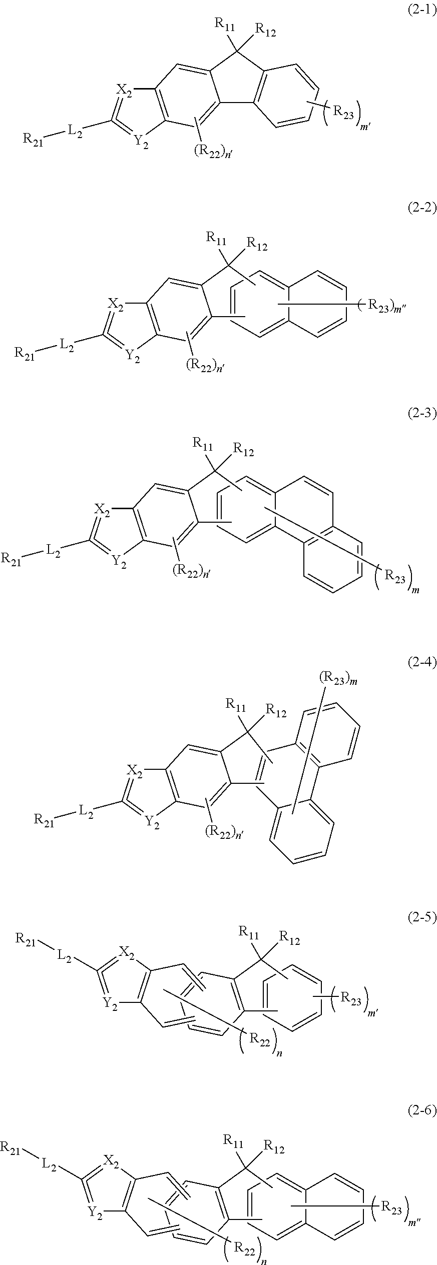

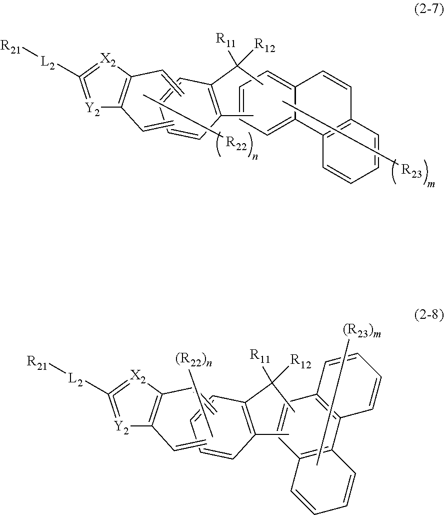

In Formulas 2-1 to 2-8,
X₂, Y₂, R₁₁, R₁₂, R₂₁ to R₂₃, L₂, n, and m are as defined in Formula 2; and
n′ represents an integer of 1 or 2, m′ represents an integer of 1 to 4, and m″ represents an integer of 1 to 6.

According to another embodiment, the host compound represented by Formula 2 may be represented by the following Formula 2-1 or 2-9.

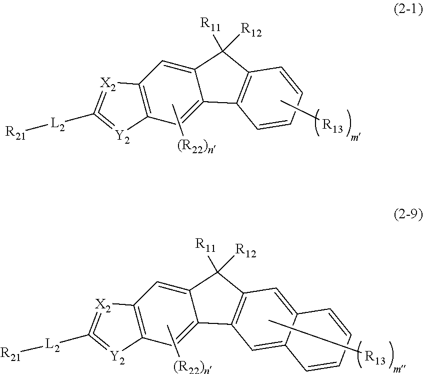

In Formulas 2-1 and 2-9,
X₂, Y₂, R₁₁, R₁₂, R₂₁ to R₂₃, and L₂ are as defined in Formula 2; and
n′ represents an integer of 1 or 2, m′ represents an integer of 1 to 4, and m″ represents an integer of 1 to 6.

In one embodiment, R₁₁ and R₁₂ each independently may be a substituted or unsubstituted (C₁-C₃₀)alkyl, preferably a substituted or unsubstituted (C₁-C₁₀)alkyl, more preferably a substituted or unsubstituted (C₁-C₄)alkyl. For example, R₁₁ and R₁₂ may be a substituted or unsubstituted methyl.

In one embodiment, any one of X₂ and Y₂ may be —N=, and the other of X₂ and Y₂ may be —NR₂₄—, —O—, or —S—.

In one embodiment, R₂₄ may be a substituted or unsubstituted (C₆-C₃₀)aryl, for example, a substituted or unsubstituted phenyl.

In one embodiment, L₂ may be a single bond.

In one embodiment, R₂₁ may be a substituted or unsubstituted (C₆-C₃₀)aryl, for example, phenyl unsubstituted or substituted with deuterium or a substituted or unsubstituted naphthyl.

In one embodiment, R₂₂ and R₂₃ each independently may be hydrogen, deuterium, halogen, cyano, a substituted or unsubstituted (5- to 30-membered)heteroaryl, or —L₃-HAr, preferably hydrogen, deuterium, a substituted or unsubstituted (5- to 25-membered)heteroaryl, or —L₃-HAr, more preferably hydrogen, a substituted or unsubstituted (5- to 18-membered)heteroaryl, or —L₃-HAr. For example, at least one of R₂₂ and R₂₃ may be —L₃-HAr.

In one embodiment, L₃ may be a single bond or a substituted or unsubstituted (C₆-C₃₀)arylene, preferably a single bond or a substituted or unsubstituted (C₆-C₂₅)arylene, more preferably a single bond or a substituted or unsubstituted (C₆-C₁₈)arylene. For example, L₃ may be a single bond or a substituted or unsubstituted phenylene.

In one embodiment, HAr may be a substituted or unsubstituted (5- to 30-membered)heteroaryl containing at least one nitrogen atom, preferably a substituted or unsubstituted (5- to 30-membered)heteroaryl containing at least two nitrogen atoms, more preferably (5- to 30-membered)heteroaryl containing at least one nitrogen atom, unsubstituted or substituted with (C₆-C₃₀)aryl.

In one embodiment, HAr may be represented by the following Formula 3 or 4.

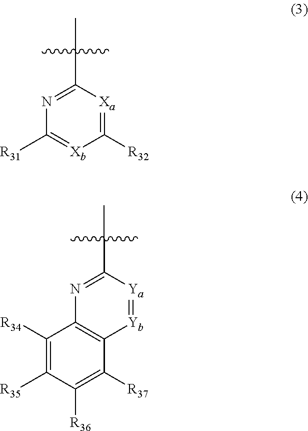

In Formulas 3 and 4,
Xₐ, X_{b}, Yₐ, and Y_{b} each independently represent N or CR₃₃; provided that at least one of Xₐ and X_{b} is N, and at least one of Yₐ and Y_{b} is N; and
R₃₁ to R₃₇ each independently represent hydrogen, deuterium, halogen, cyano, a substituted or unsubstituted (C₁-C₃₀)alkyl, a substituted or unsubstituted (C₆-C₅₀)aryl, a substituted or unsubstituted (3- to 50-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted mono- or di(C₁-C₃₀)alkylamino, a substituted or unsubstituted mono- or di(C₆-C₃₀)arylamino, or a substituted or unsubstituted (C₁-C₃₀)alkyl(C₆-C₃₀)arylamino; or may be linked to the adjacent substituent to form a ring(s).

In one embodiment, Xₐ may be N, and X_{b} may be CR₃₃.

In one embodiment, all of Xₐ and X_{b} may be N.

In one embodiment, R₃₁ and R₃₂ each independently may be a substituted or unsubstituted (C₆-C₅₀)aryl or a substituted or unsubstituted (5- to 50-membered)heteroaryl, preferably a substituted or unsubstituted (C₆-C₃₀)aryl or a substituted or unsubstituted (5- to 30-membered)heteroaryl, more preferably a substituted or unsubstituted (C₆-C₂₅)aryl or a substituted or unsubstituted (5- to 25-membered)heteroaryl. For example, R₃₁ and R₃₂ each independently may be a substituted or unsubstituted phenyl, a substituted or unsubstituted p-biphenyl, a substituted or unsubstituted naphthyl, a substituted or unsubstituted dibenzofuranyl, a substituted or unsubstituted dibenzothiophenyl, or carbazolyl unsubstituted or unsubstituted with phenyl.

In one embodiment, Yₐ may be N, and Y_{b} may be CR₃₃.

In one embodiment, Yₐ may be CR₃₃, and Y_{b} may be N.

In one embodiment, R₃₄ to R₃₇ each independently may be hydrogen or deuterium, preferably hydrogen.

In one embodiment, R₃₃ may be hydrogen or a substituted or unsubstituted (C₆-C₃₀)aryl, for example, hydrogen or a substituted or unsubstituted phenyl.

According to one embodiment, the compound represented by Formula 2 above may be more specifically illustrated by the following compounds, but is not limited thereto.

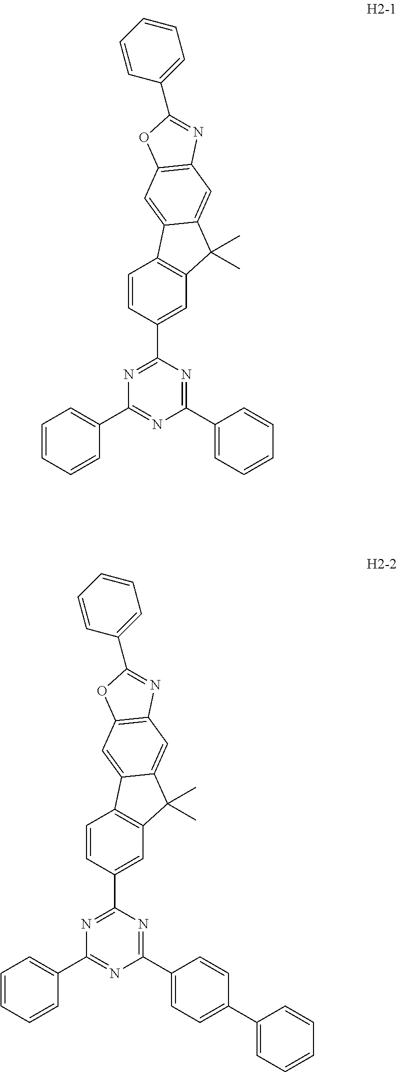 [SMILES: CC1(C)c2cc(-c3nc(-c4ccccc4)nc(-c4ccccc4)n3)ccc2-c2cc3oc(-c4ccccc4)nc3cc21 ; CC1(C)c2cc(-c3nc(-c4ccccc4)nc(-c4ccc(-c5ccccc5)cc4)n3)ccc2-c2cc3oc(-c4ccccc4)nc3cc21]

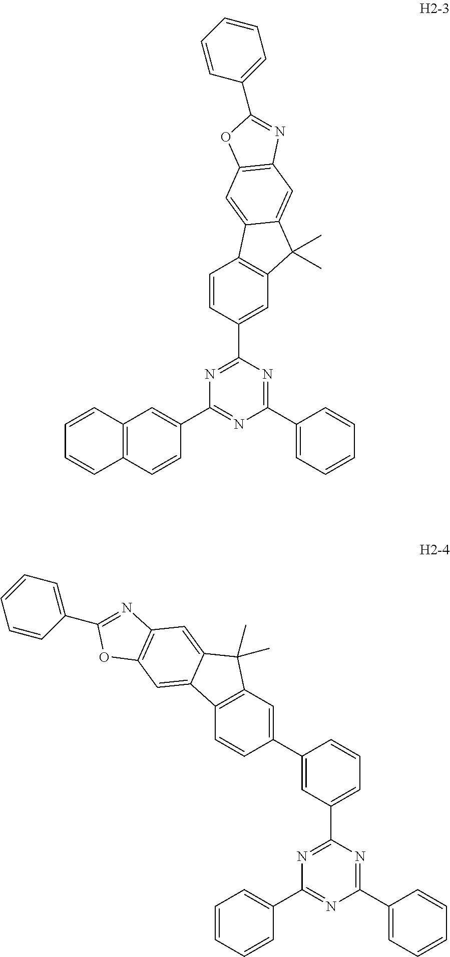 [SMILES: CC1(C)c2cc(-c3nc(-c4ccccc4)nc(-c4ccc5ccccc5c4)n3)ccc2-c2cc3oc(-c4ccccc4)nc3cc21 ; CC1(C)c2cc(-c3cccc(-c4nc(-c5ccccc5)nc(-c5ccccc5)n4)c3)ccc2-c2cc3oc(-c4ccccc4)nc3cc21]

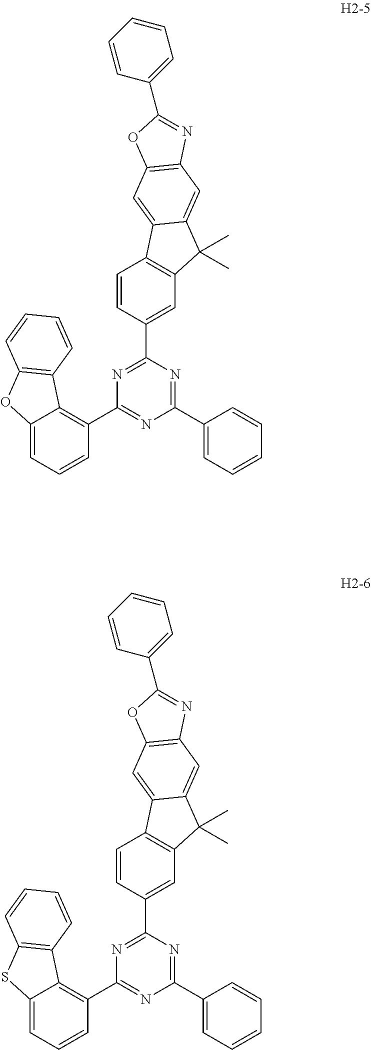 [SMILES: CC1(C)c2cc(-c3nc(-c4ccccc4)nc(-c4cccc5oc6ccccc6c45)n3)ccc2-c2cc3oc(-c4ccccc4)nc3cc21 ; CC1(C)c2cc(-c3nc(-c4ccccc4)nc(-c4cccc5sc6ccccc6c45)n3)ccc2-c2cc3oc(-c4ccccc4)nc3cc21]

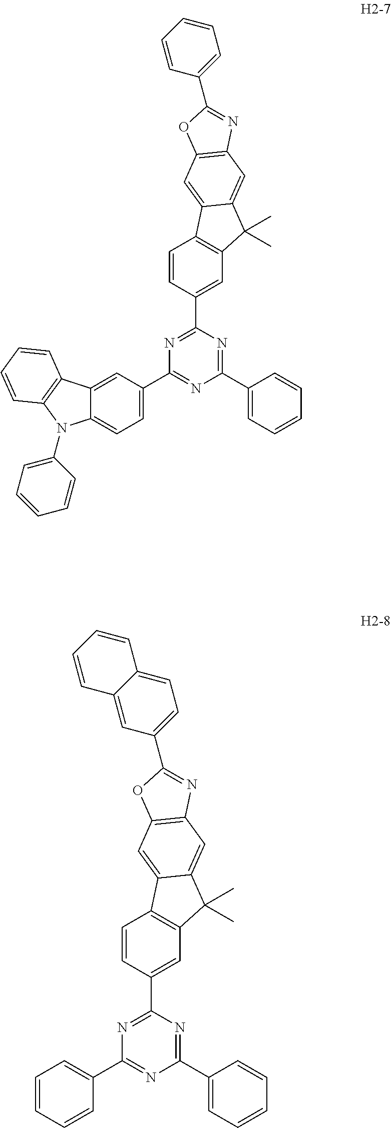 [SMILES: CC1(C)c2cc(-c3nc(-c4ccccc4)nc(-c4ccc5c(c4)c4ccccc4n5-c4ccccc4)n3)ccc2-c2cc3oc(-c4ccccc4)nc3cc21 ; CC1(C)c2cc(-c3nc(-c4ccccc4)nc(-c4ccccc4)n3)ccc2-c2cc3oc(-c4ccc5ccccc5c4)nc3cc21]

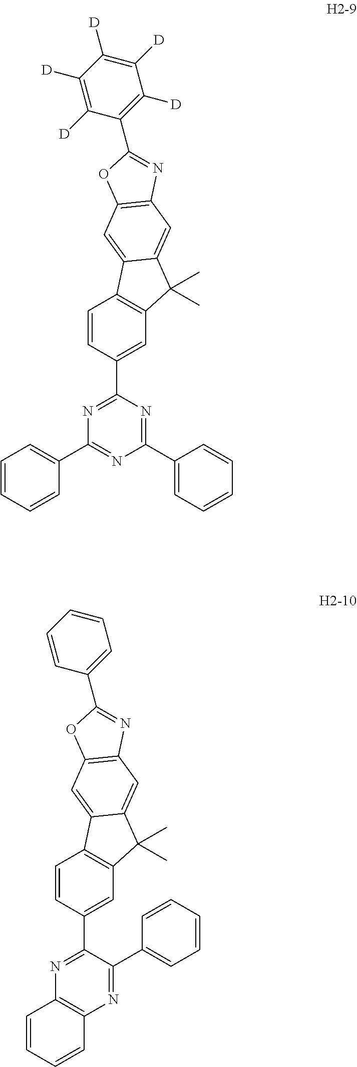 [SMILES: [2H]c1c([2H])c([2H])c(-c2nc3cc4c(cc3o2)-c2ccc(-c3nc(-c5ccccc5)nc(-c5ccccc5)n3)cc2C4(C)C)c([2H])c1[2H] ; CC1(C)c2cc(-c3nc4ccccc4nc3-c3ccccc3)ccc2-c2cc3oc(-c4ccccc4)nc3cc21]

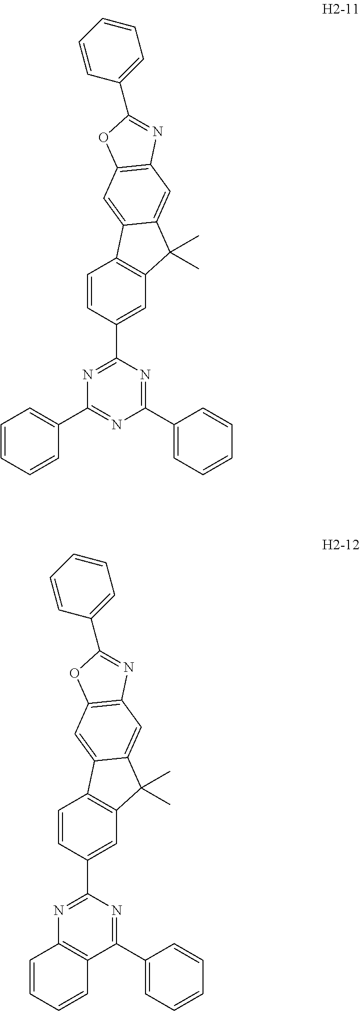 [SMILES: CC1(C)c2cc(-c3nc(-c4ccccc4)nc(-c4ccccc4)n3)ccc2-c2cc3oc(-c4ccccc4)nc3cc21 ; CC1(C)c2cc(-c3nc(-c4ccccc4)c4ccccc4n3)ccc2-c2cc3oc(-c4ccccc4)nc3cc21]

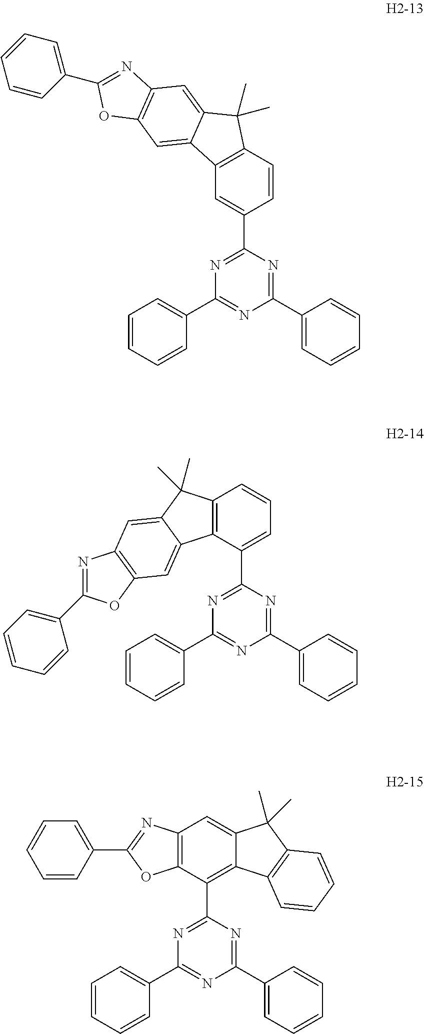 [SMILES: CC1(C)c2ccc(-c3nc(-c4ccccc4)nc(-c4ccccc4)n3)cc2-c2cc3oc(-c4ccccc4)nc3cc21 ; CC1(C)c2cc3nc(-c4ccccc4)oc3cc2-c2c(-c3nc(-c4ccccc4)nc(-c4ccccc4)n3)cccc21 ; CC1(C)c2ccccc2-c2c1cc1nc(-c3ccccc3)oc1c2-c1nc(-c2ccccc2)nc(-c2ccccc2)n1]

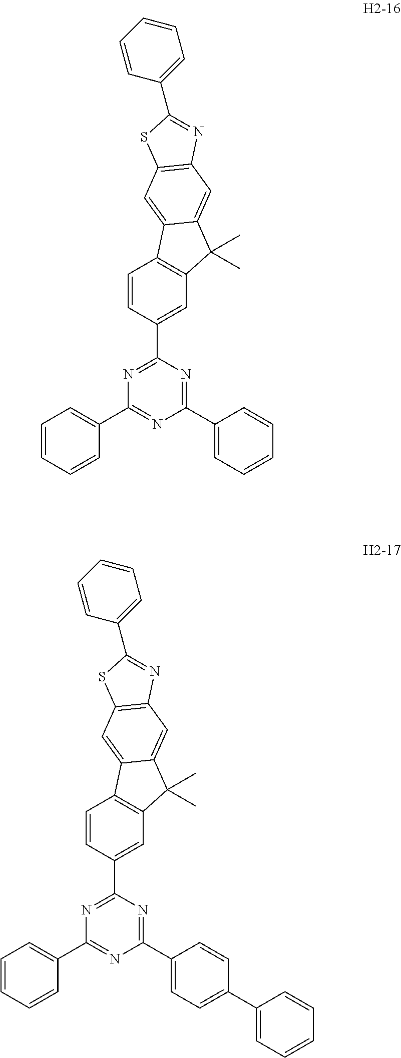 [SMILES: CC1(C)c2cc(-c3nc(-c4ccccc4)nc(-c4ccccc4)n3)ccc2-c2cc3sc(-c4ccccc4)nc3cc21 ; CC1(C)c2cc(-c3nc(-c4ccccc4)nc(-c4ccc(-c5ccccc5)cc4)n3)ccc2-c2cc3sc(-c4ccccc4)nc3cc21]

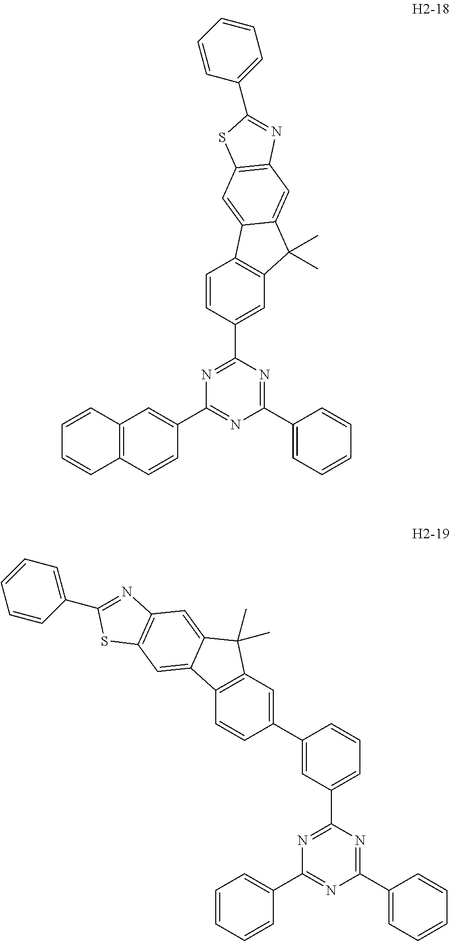 [SMILES: CC1(C)c2cc(-c3nc(-c4ccccc4)nc(-c4ccc5ccccc5c4)n3)ccc2-c2cc3sc(-c4ccccc4)nc3cc21 ; CC1(C)c2cc(-c3cccc(-c4nc(-c5ccccc5)nc(-c5ccccc5)n4)c3)ccc2-c2cc3sc(-c4ccccc4)nc3cc21]

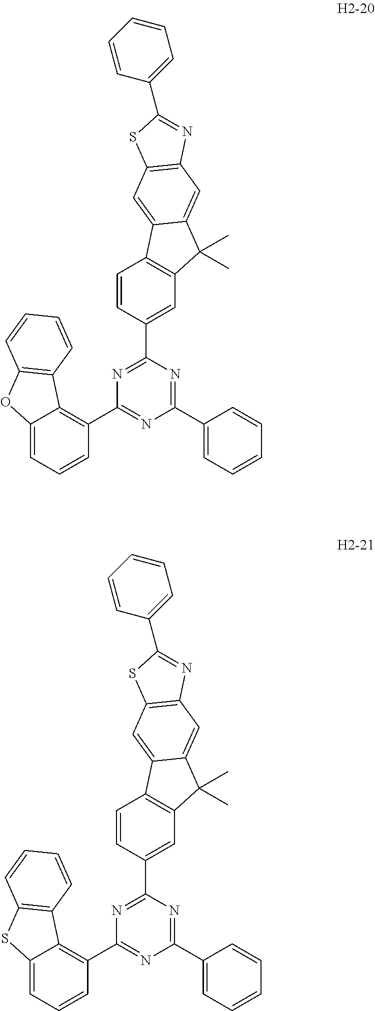 [SMILES: CC1(C)c2cc(-c3nc(-c4ccccc4)nc(-c4cccc5oc6ccccc6c45)n3)ccc2-c2cc3sc(-c4ccccc4)nc3cc21 ; CC1(C)c2cc(-c3nc(-c4ccccc4)nc(-c4cccc5sc6ccccc6c45)n3)ccc2-c2cc3sc(-c4ccccc4)nc3cc21]

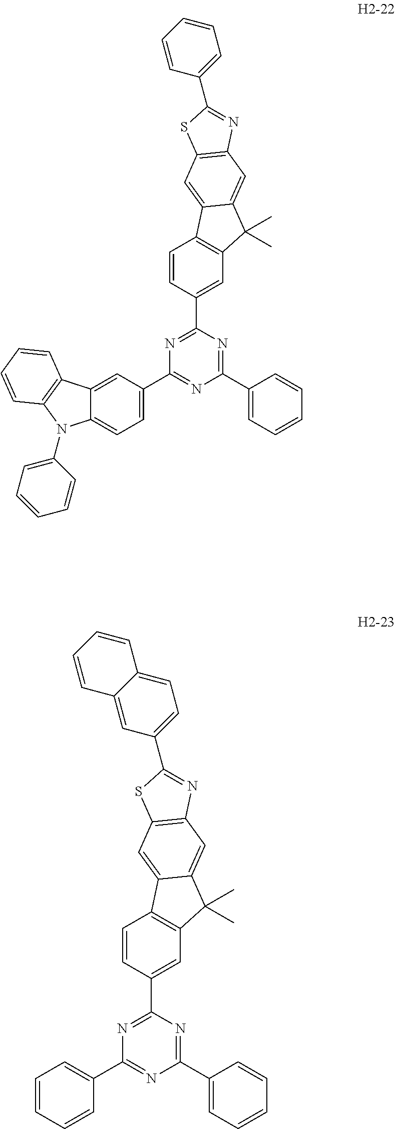 [SMILES: CC1(C)c2cc(-c3nc(-c4ccccc4)nc(-c4ccc5c(c4)c4ccccc4n5-c4ccccc4)n3)ccc2-c2cc3sc(-c4ccccc4)nc3cc21 ; CC1(C)c2cc(-c3nc(-c4ccccc4)nc(-c4ccccc4)n3)ccc2-c2cc3sc(-c4ccc5ccccc5c4)nc3cc21]

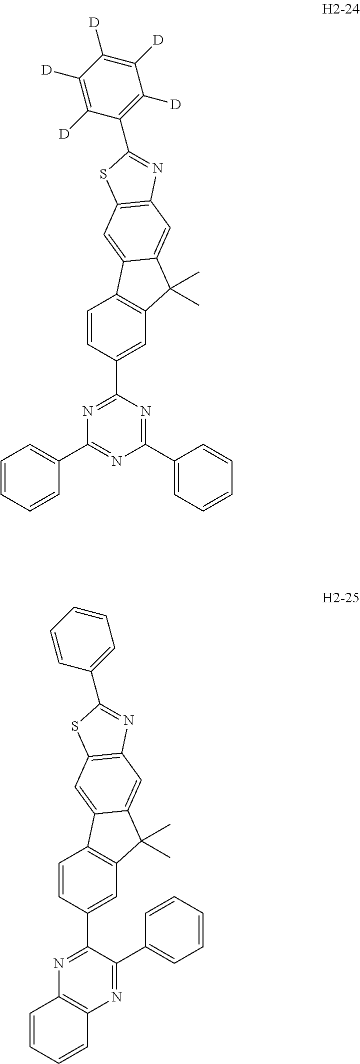 [SMILES: [2H]c1c([2H])c([2H])c(-c2nc3cc4c(cc3s2)-c2ccc(-c3nc(-c5ccccc5)nc(-c5ccccc5)n3)cc2C4(C)C)c([2H])c1[2H] ; CC1(C)c2cc(-c3nc4ccccc4nc3-c3ccccc3)ccc2-c2cc3sc(-c4ccccc4)nc3cc21]

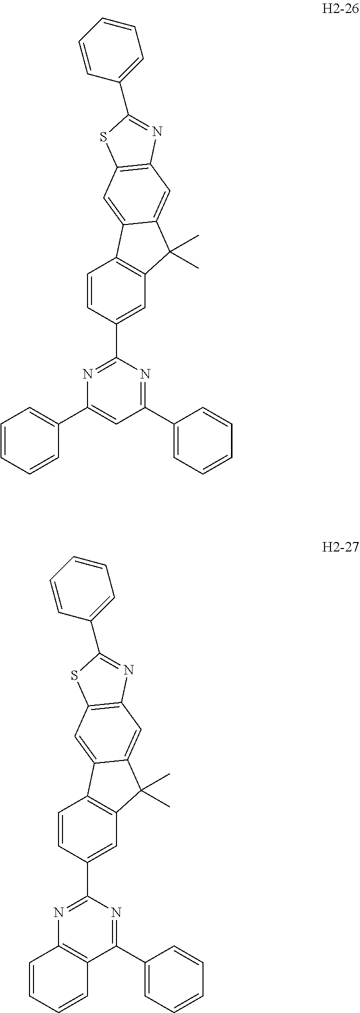 [SMILES: CC1(C)c2cc(-c3nc(-c4ccccc4)cc(-c4ccccc4)n3)ccc2-c2cc3sc(-c4ccccc4)nc3cc21 ; CC1(C)c2cc(-c3nc(-c4ccccc4)c4ccccc4n3)ccc2-c2cc3sc(-c4ccccc4)nc3cc21]

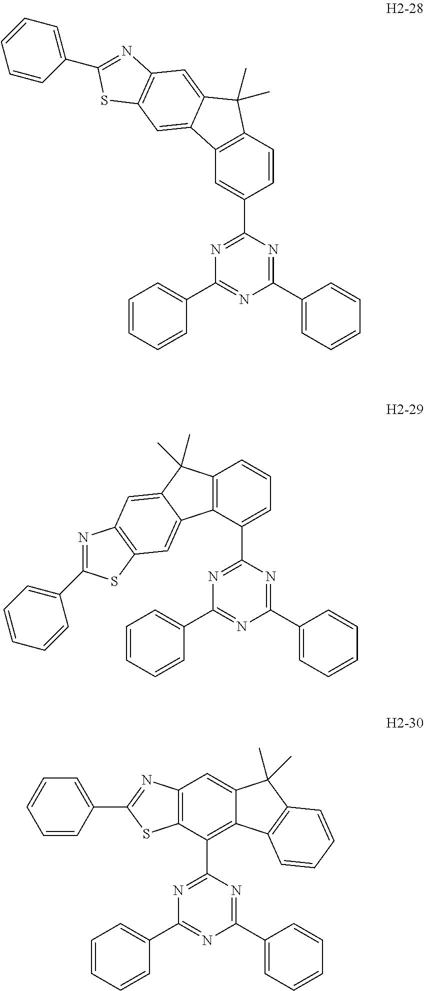 [SMILES: CC1(C)c2ccc(-c3nc(-c4ccccc4)nc(-c4ccccc4)n3)cc2-c2cc3sc(-c4ccccc4)nc3cc21 ; CC1(C)c2cc3nc(-c4ccccc4)sc3cc2-c2c(-c3nc(-c4ccccc4)nc(-c4ccccc4)n3)cccc21 ; CC1(C)c2ccccc2-c2c1cc1nc(-c3ccccc3)sc1c2-c1nc(-c2ccccc2)nc(-c2ccccc2)n1]

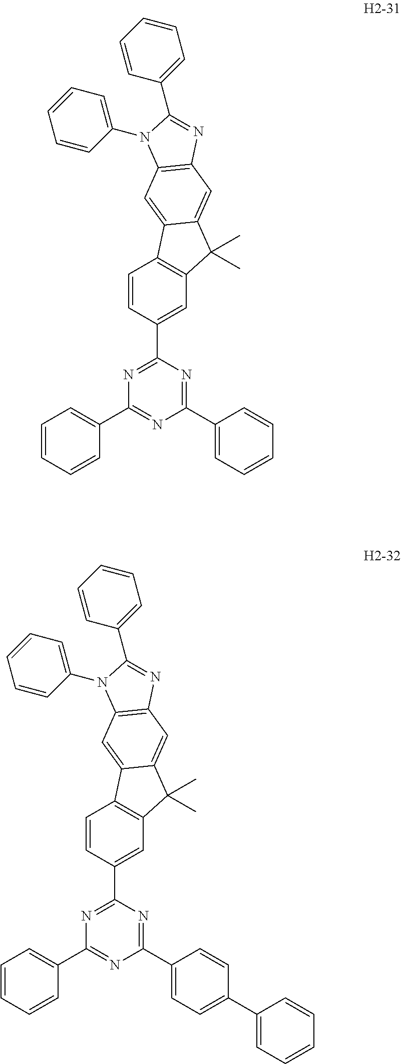 [SMILES: CC1(C)c2cc(-c3nc(-c4ccccc4)nc(-c4ccccc4)n3)ccc2-c2cc3c(cc21)nc(-c1ccccc1)n3-c1ccccc1 ; CC1(C)c2cc(-c3nc(-c4ccccc4)nc(-c4ccc(-c5ccccc5)cc4)n3)ccc2-c2cc3c(cc21)nc(-c1ccccc1)n3-c1ccccc1]

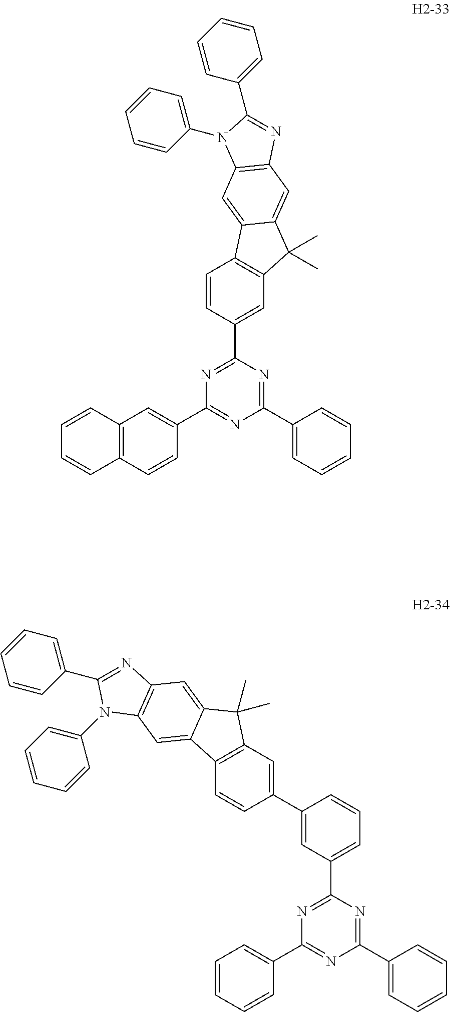 [SMILES: CC1(C)c2cc(-c3nc(-c4ccccc4)nc(-c4ccc5ccccc5c4)n3)ccc2-c2cc3c(cc21)nc(-c1ccccc1)n3-c1ccccc1 ; CC1(C)c2cc(-c3cccc(-c4nc(-c5ccccc5)nc(-c5ccccc5)n4)c3)ccc2-c2cc3c(cc21)nc(-c1ccccc1)n3-c1ccccc1]

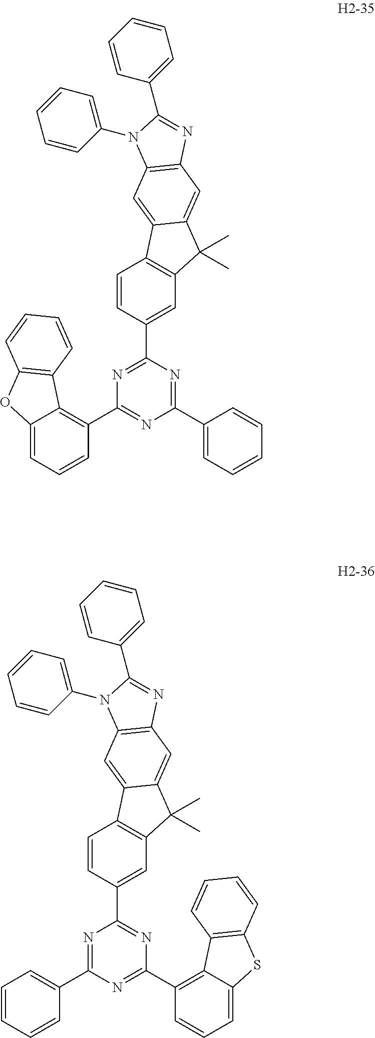 [SMILES: CC1(C)c2cc(-c3nc(-c4ccccc4)nc(-c4cccc5oc6ccccc6c45)n3)ccc2-c2cc3c(cc21)nc(-c1ccccc1)n3-c1ccccc1 ; CC1(C)c2cc(-c3nc(-c4ccccc4)nc(-c4cccc5sc6ccccc6c45)n3)ccc2-c2cc3c(cc21)nc(-c1ccccc1)n3-c1ccccc1]

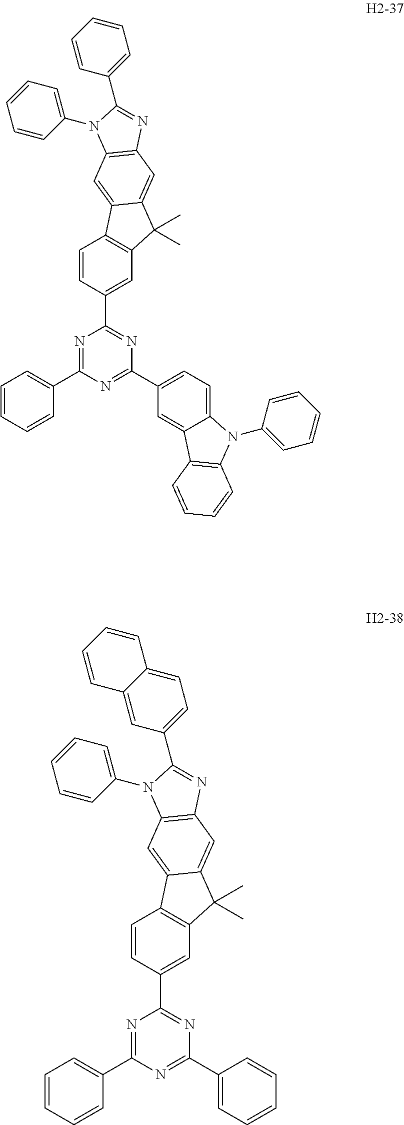 [SMILES: CC1(C)c2cc(-c3nc(-c4ccccc4)nc(-c4ccc5c(c4)c4ccccc4n5-c4ccccc4)n3)ccc2-c2cc3c(cc21)nc(-c1ccccc1)n3-c1ccccc1 ; CC1(C)c2cc(-c3nc(-c4ccccc4)nc(-c4ccccc4)n3)ccc2-c2cc3c(cc21)nc(-c1ccc2ccccc2c1)n3-c1ccccc1]

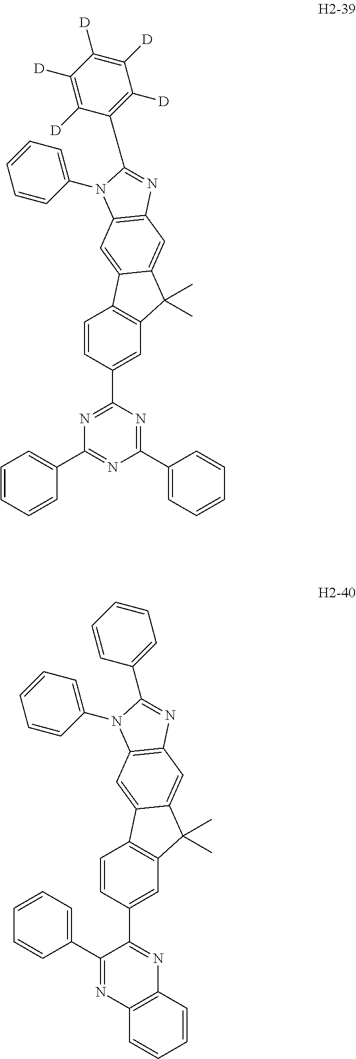 [SMILES: [2H]c1c([2H])c([2H])c(-c2nc3cc4c(cc3n2-c2ccccc2)-c2ccc(-c3nc(-c5ccccc5)nc(-c5ccccc5)n3)cc2C4(C)C)c([2H])c1[2H] ; CC1(C)c2cc(-c3nc4ccccc4nc3-c3ccccc3)ccc2-c2cc3c(cc21)nc(-c1ccccc1)n3-c1ccccc1]

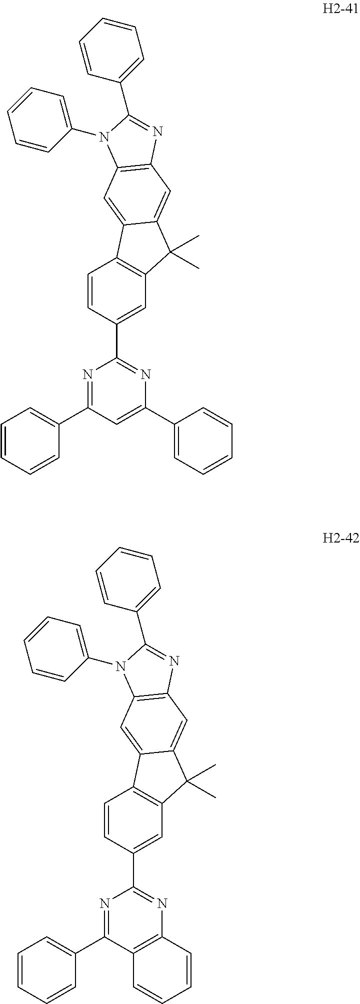 [SMILES: CC1(C)c2cc(-c3nc(-c4ccccc4)cc(-c4ccccc4)n3)ccc2-c2cc3c(cc21)nc(-c1ccccc1)n3-c1ccccc1 ; CC1(C)c2cc(-c3nc(-c4ccccc4)c4ccccc4n3)ccc2-c2cc3c(cc21)nc(-c1ccccc1)n3-c1ccccc1]

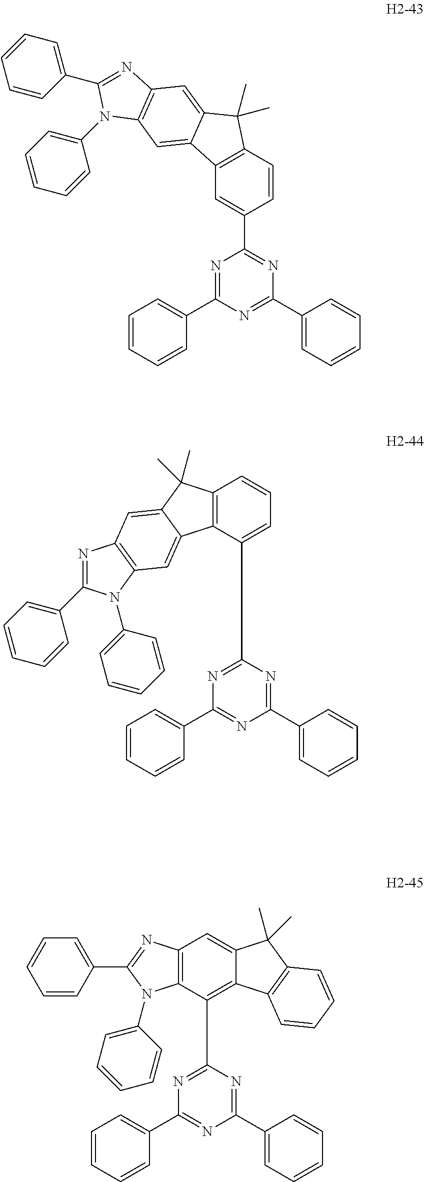 [SMILES: CC1(C)c2ccc(-c3nc(-c4ccccc4)nc(-c4ccccc4)n3)cc2-c2cc3c(cc21)nc(-c1ccccc1)n3-c1ccccc1 ; CC1(C)c2cc3nc(-c4ccccc4)n(-c4ccccc4)c3cc2-c2c(-c3nc(-c4ccccc4)nc(-c4ccccc4)n3)cccc21 ; CC1(C)c2ccccc2-c2c1cc1nc(-c3ccccc3)n(-c3ccccc3)c1c2-c1nc(-c2ccccc2)nc(-c2ccccc2)n1]

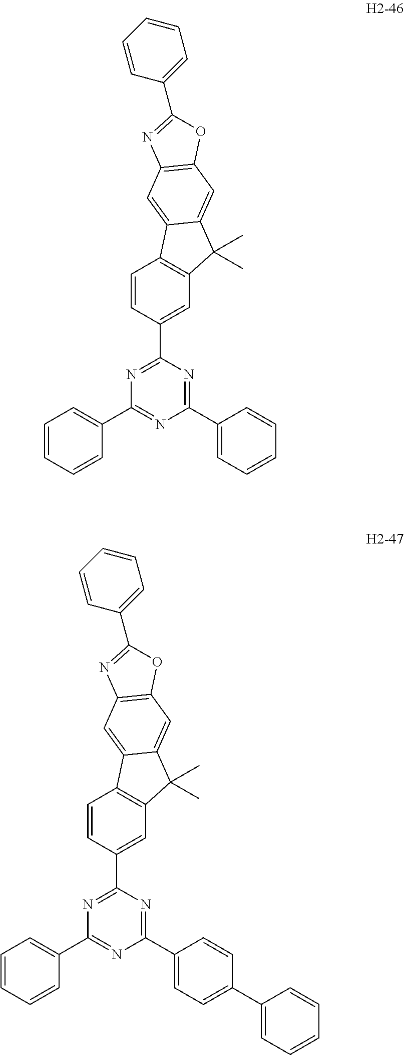 [SMILES: CC1(C)c2cc(-c3nc(-c4ccccc4)nc(-c4ccccc4)n3)ccc2-c2cc3nc(-c4ccccc4)oc3cc21 ; CC1(C)c2cc(-c3nc(-c4ccccc4)nc(-c4ccc(-c5ccccc5)cc4)n3)ccc2-c2cc3nc(-c4ccccc4)oc3cc21]

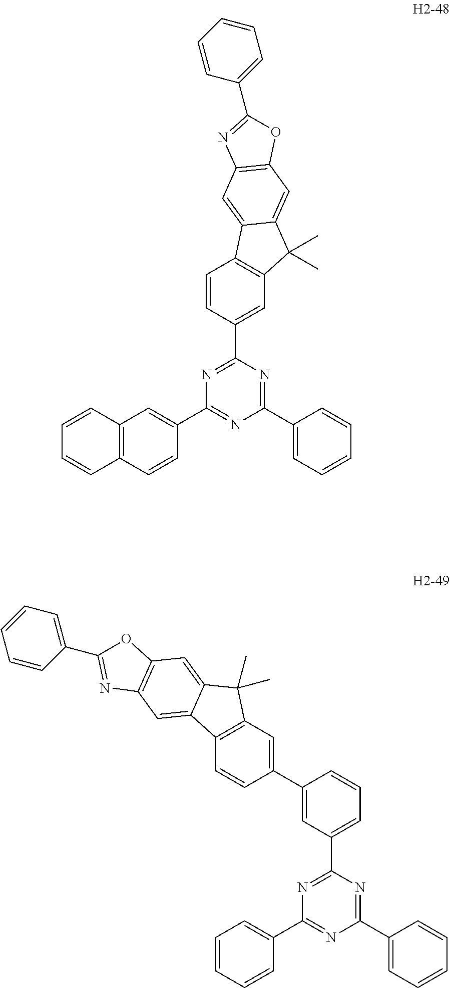 [SMILES: CC1(C)c2cc(-c3nc(-c4ccccc4)nc(-c4ccc5ccccc5c4)n3)ccc2-c2cc3nc(-c4ccccc4)oc3cc21 ; CC1(C)c2cc(-c3cccc(-c4nc(-c5ccccc5)nc(-c5ccccc5)n4)c3)ccc2-c2cc3nc(-c4ccccc4)oc3cc21]

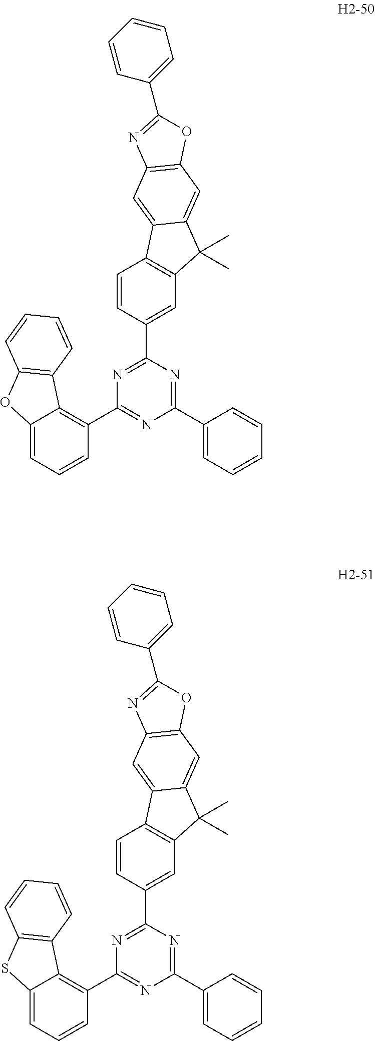 [SMILES: CC1(C)c2cc(-c3nc(-c4ccccc4)nc(-c4cccc5oc6ccccc6c45)n3)ccc2-c2cc3nc(-c4ccccc4)oc3cc21 ; CC1(C)c2cc(-c3nc(-c4ccccc4)nc(-c4cccc5sc6ccccc6c45)n3)ccc2-c2cc3nc(-c4ccccc4)oc3cc21]

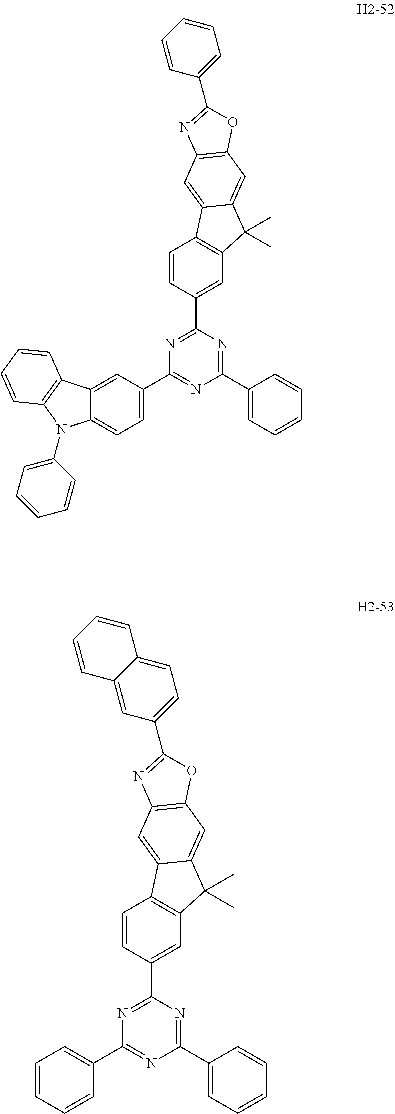 [SMILES: CC1(C)c2cc(-c3nc(-c4ccccc4)nc(-c4ccc5c(c4)c4ccccc4n5-c4ccccc4)n3)ccc2-c2cc3nc(-c4ccccc4)oc3cc21 ; CC1(C)c2cc(-c3nc(-c4ccccc4)nc(-c4ccccc4)n3)ccc2-c2cc3nc(-c4ccc5ccccc5c4)oc3cc21]

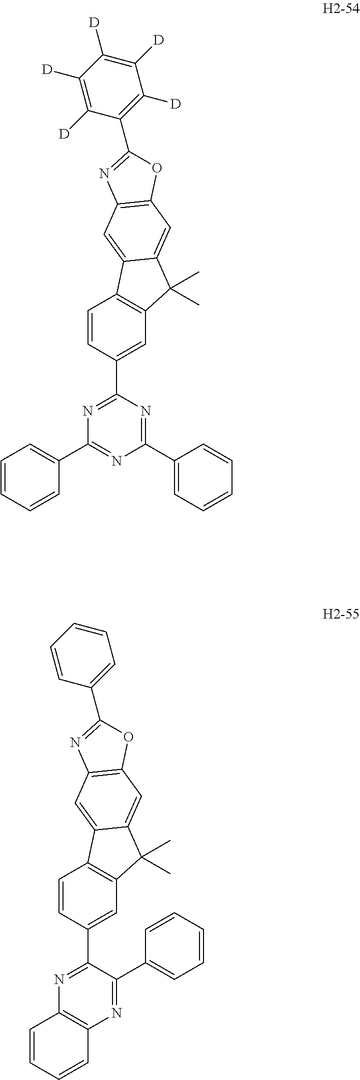 [SMILES: [2H]c1c([2H])c([2H])c(-c2nc3cc4c(cc3o2)C(C)(C)c2cc(-c3nc(-c5ccccc5)nc(-c5ccccc5)n3)ccc2-4)c([2H])c1[2H] ; CC1(C)c2cc(-c3nc4ccccc4nc3-c3ccccc3)ccc2-c2cc3nc(-c4ccccc4)oc3cc21]

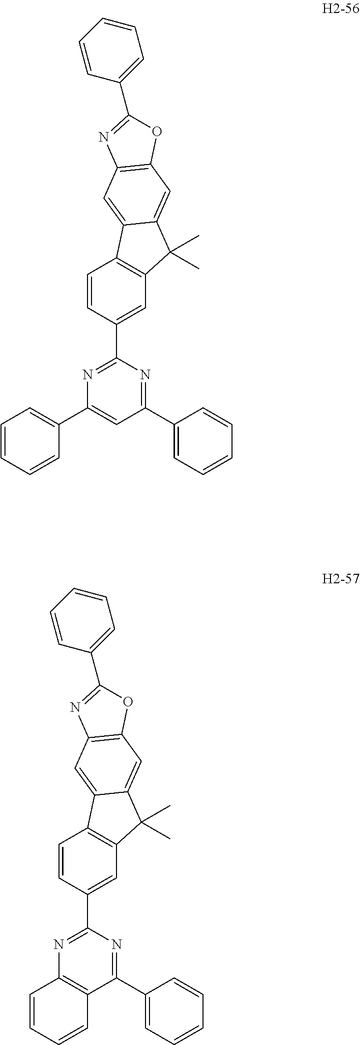 [SMILES: CC1(C)c2cc(-c3nc(-c4ccccc4)cc(-c4ccccc4)n3)ccc2-c2cc3nc(-c4ccccc4)oc3cc21 ; CC1(C)c2cc(-c3nc(-c4ccccc4)c4ccccc4n3)ccc2-c2cc3nc(-c4ccccc4)oc3cc21]

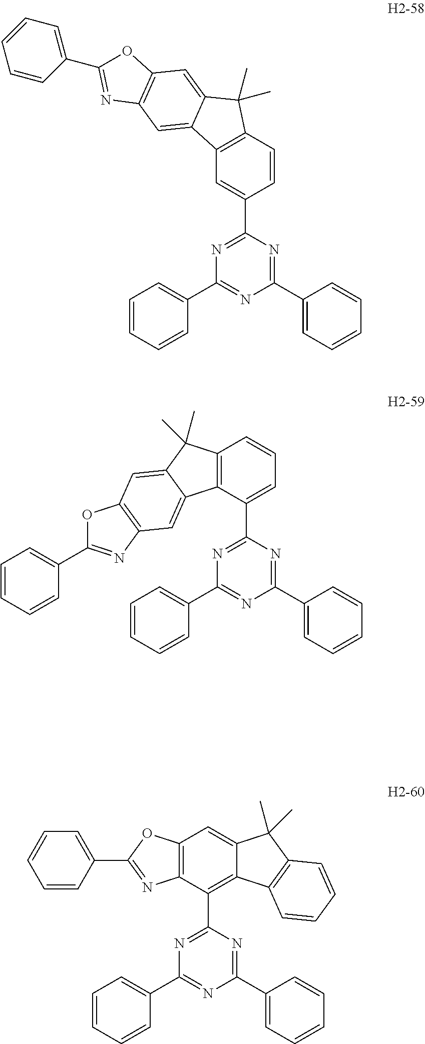 [SMILES: CC1(C)c2ccc(-c3nc(-c4ccccc4)nc(-c4ccccc4)n3)cc2-c2cc3nc(-c4ccccc4)oc3cc21 ; CC1(C)c2cc3oc(-c4ccccc4)nc3cc2-c2c(-c3nc(-c4ccccc4)nc(-c4ccccc4)n3)cccc21 ; CC1(C)c2ccccc2-c2c1cc1oc(-c3ccccc3)nc1c2-c1nc(-c2ccccc2)nc(-c2ccccc2)n1]

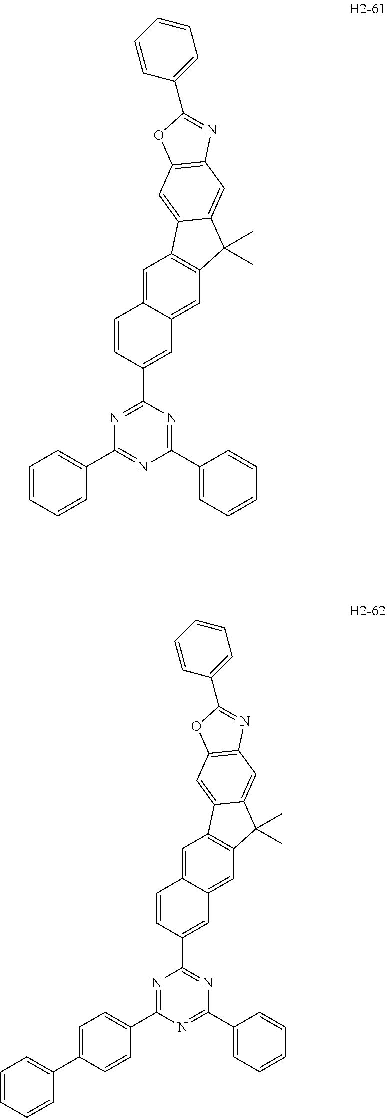 [SMILES: CC1(C)c2cc3cc(-c4nc(-c5ccccc5)nc(-c5ccccc5)n4)ccc3cc2-c2cc3oc(-c4ccccc4)nc3cc21 ; CC1(C)c2cc3cc(-c4nc(-c5ccccc5)nc(-c5ccc(-c6ccccc6)cc5)n4)ccc3cc2-c2cc3oc(-c4ccccc4)nc3cc21]

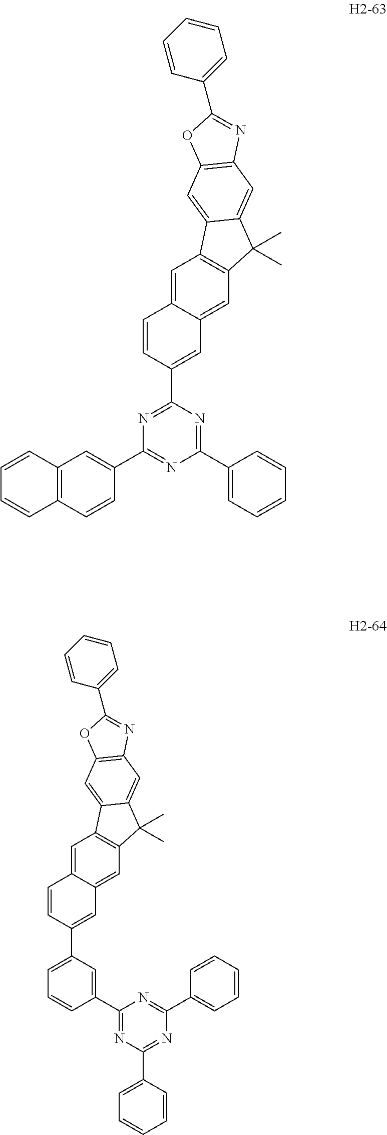 [SMILES: CC1(C)c2cc3cc(-c4nc(-c5ccccc5)nc(-c5ccc6ccccc6c5)n4)ccc3cc2-c2cc3oc(-c4ccccc4)nc3cc21 ; CC1(C)c2cc3cc(-c4cccc(-c5nc(-c6ccccc6)nc(-c6ccccc6)n5)c4)ccc3cc2-c2cc3oc(-c4ccccc4)nc3cc21]

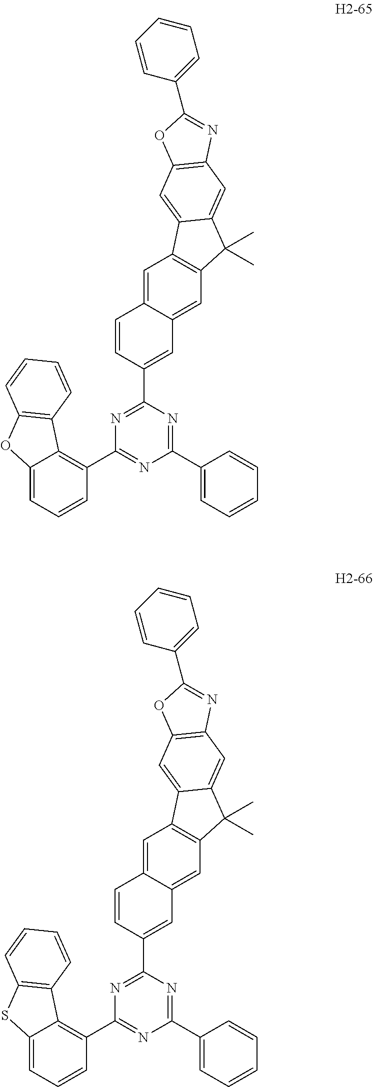 [SMILES: CC1(C)c2cc3cc(-c4nc(-c5ccccc5)nc(-c5cccc6oc7ccccc7c56)n4)ccc3cc2-c2cc3oc(-c4ccccc4)nc3cc21 ; CC1(C)c2cc3cc(-c4nc(-c5ccccc5)nc(-c5cccc6sc7ccccc7c56)n4)ccc3cc2-c2cc3oc(-c4ccccc4)nc3cc21]

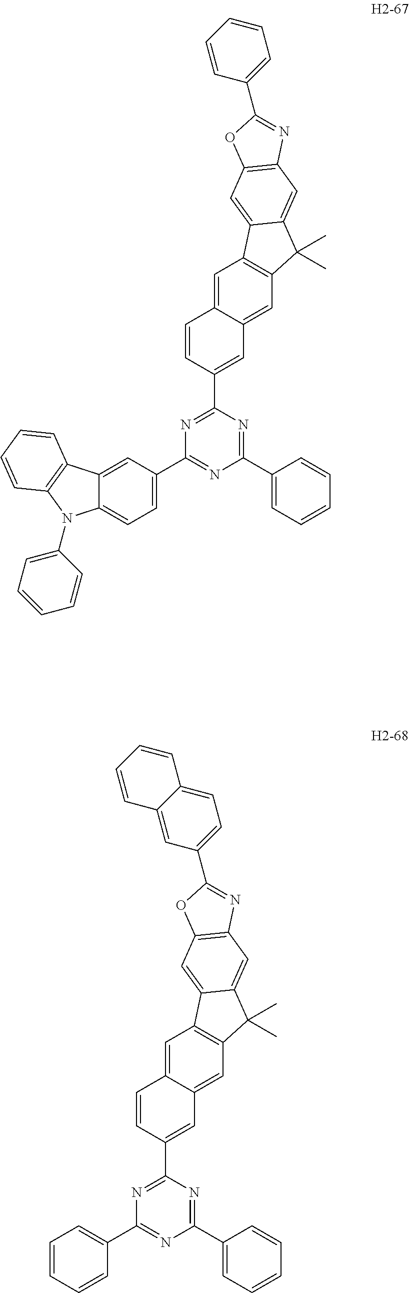 [SMILES: CC1(C)c2cc3cc(-c4nc(-c5ccccc5)nc(-c5ccc6c(c5)c5ccccc5n6-c5ccccc5)n4)ccc3cc2-c2cc3oc(-c4ccccc4)nc3cc21 ; CC1(C)c2cc3cc(-c4nc(-c5ccccc5)nc(-c5ccccc5)n4)ccc3cc2-c2cc3oc(-c4ccc5ccccc5c4)nc3cc21]

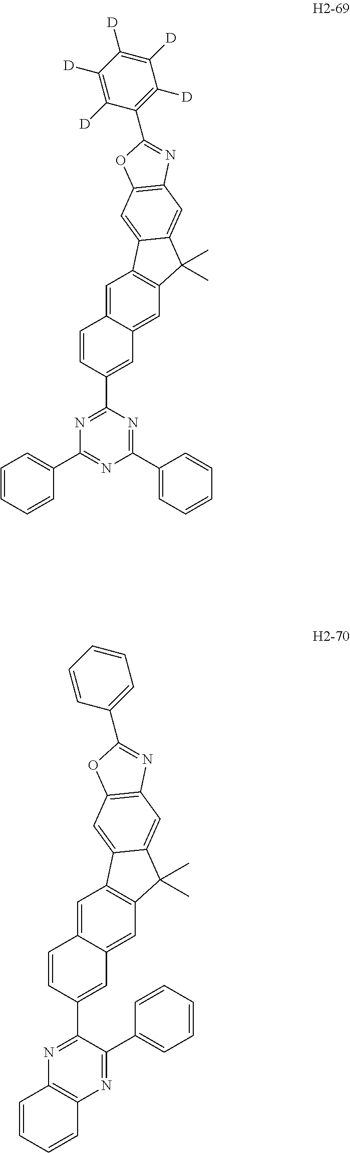 [SMILES: [2H]c1c([2H])c([2H])c(-c2nc3cc4c(cc3o2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3oc(-c4ccccc4)nc3cc21]

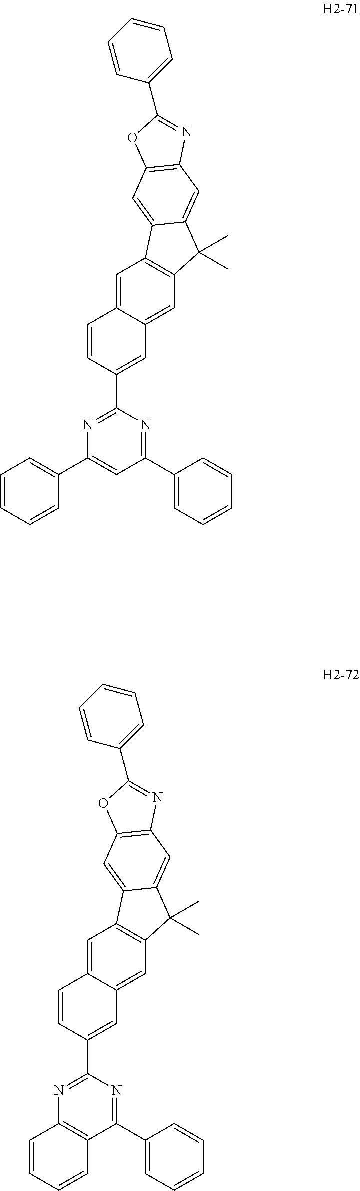 [SMILES: CC1(C)c2cc3cc(-c4nc(-c5ccccc5)cc(-c5ccccc5)n4)ccc3cc2-c2cc3oc(-c4ccccc4)nc3cc21 ; CC1(C)c2cc3cc(-c4nc(-c5ccccc5)c5ccccc5n4)ccc3cc2-c2cc3oc(-c4ccccc4)nc3cc21]

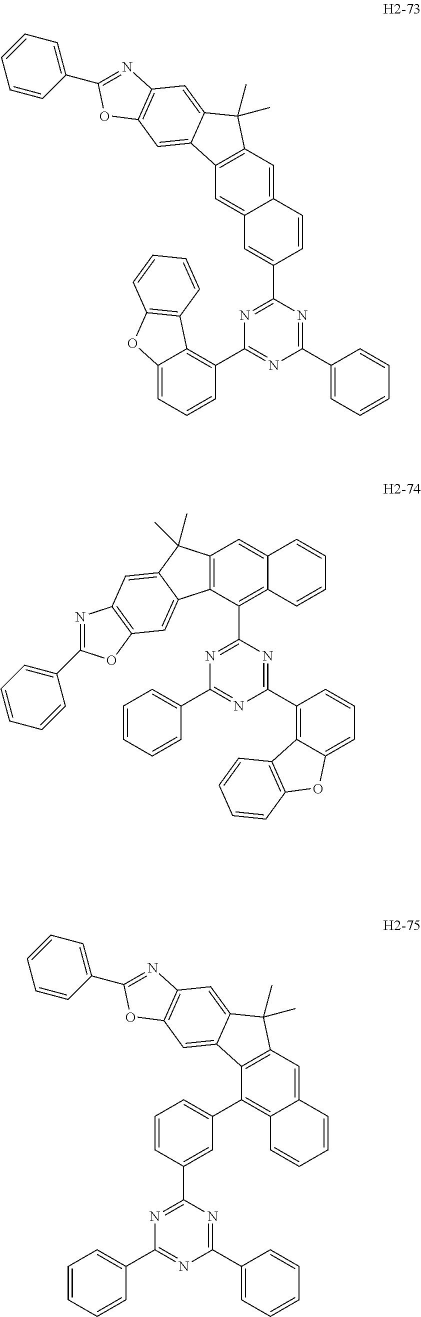 [SMILES: CC1(C)c2cc3ccc(-c4nc(-c5ccccc5)nc(-c5cccc6oc7ccccc7c56)n4)cc3cc2-c2cc3oc(-c4ccccc4)nc3cc21 ; CC1(C)c2cc3nc(-c4ccccc4)oc3cc2-c2c1cc1ccccc1c2-c1nc(-c2ccccc2)nc(-c2cccc3oc4ccccc4c23)n1 ; CC1(C)c2cc3nc(-c4ccccc4)oc3cc2-c2c1cc1ccccc1c2-c1cccc(-c2nc(-c3ccccc3)nc(-c3ccccc3)n2)c1]

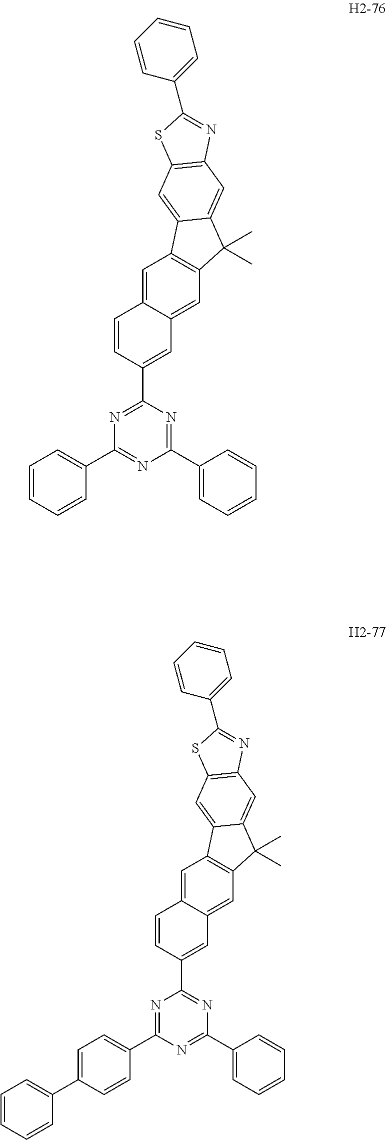 [SMILES: CC1(C)c2cc3cc(-c4nc(-c5ccccc5)nc(-c5ccccc5)n4)ccc3cc2-c2cc3sc(-c4ccccc4)nc3cc21 ; CC1(C)c2cc3cc(-c4nc(-c5ccccc5)nc(-c5ccc(-c6ccccc6)cc5)n4)ccc3cc2-c2cc3sc(-c4ccccc4)nc3cc21]

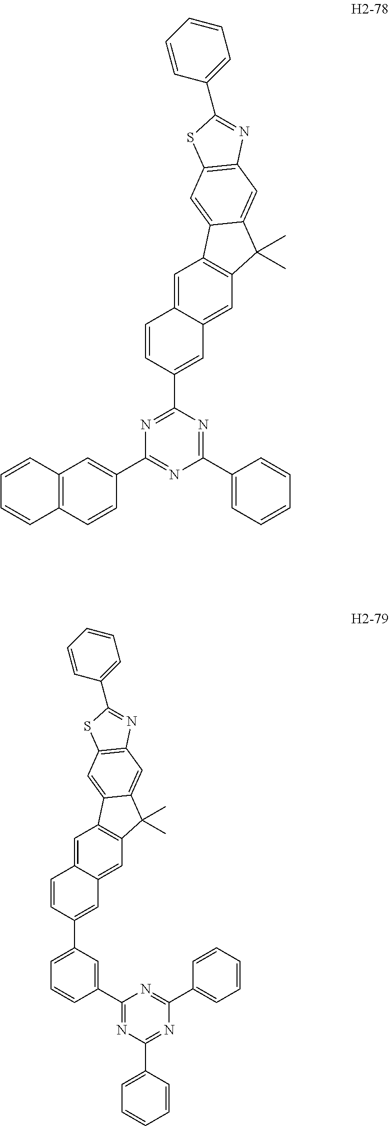 [SMILES: CC1(C)c2cc3cc(-c4nc(-c5ccccc5)nc(-c5ccc6ccccc6c5)n4)ccc3cc2-c2cc3sc(-c4ccccc4)nc3cc21 ; CC1(C)c2cc3cc(-c4cccc(-c5nc(-c6ccccc6)nc(-c6ccccc6)n5)c4)ccc3cc2-c2cc3sc(-c4ccccc4)nc3cc21]

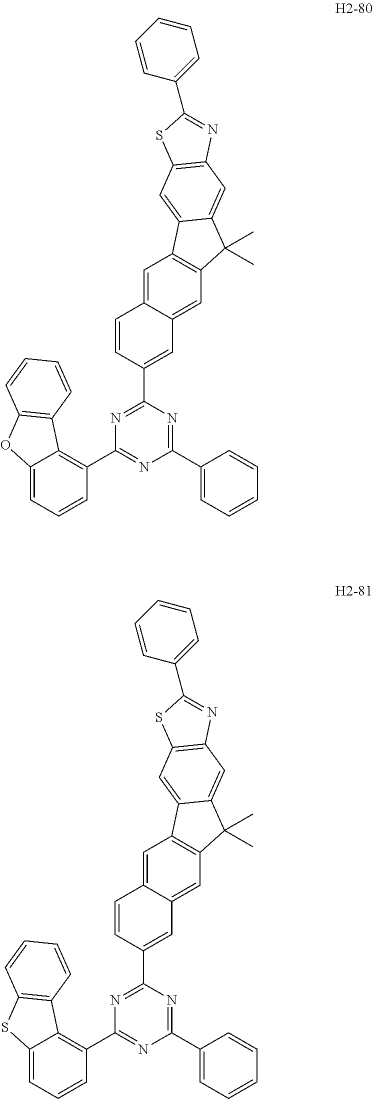 [SMILES: CC1(C)c2cc3cc(-c4nc(-c5ccccc5)nc(-c5cccc6oc7ccccc7c56)n4)ccc3cc2-c2cc3sc(-c4ccccc4)nc3cc21 ; CC1(C)c2cc3cc(-c4nc(-c5ccccc5)nc(-c5cccc6sc7ccccc7c56)n4)ccc3cc2-c2cc3sc(-c4ccccc4)nc3cc21]

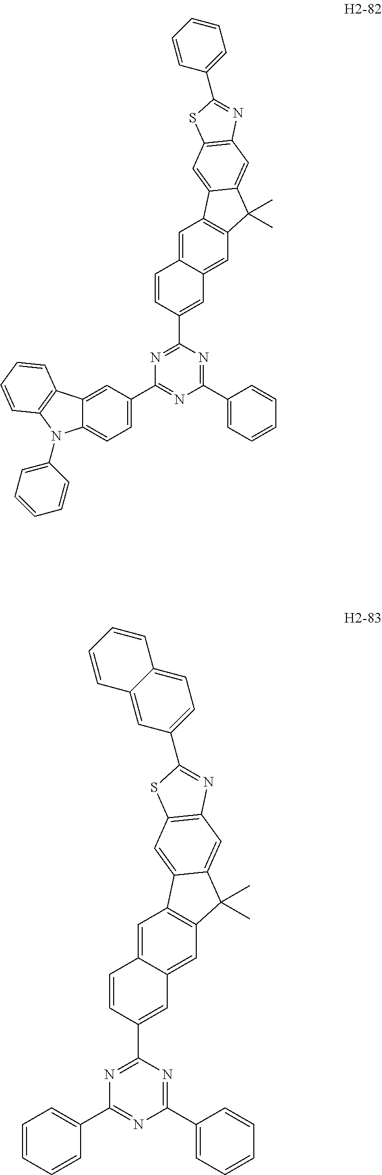 [SMILES: CC1(C)c2cc3cc(-c4nc(-c5ccccc5)nc(-c5ccc6c(c5)c5ccccc5n6-c5ccccc5)n4)ccc3cc2-c2cc3sc(-c4ccccc4)nc3cc21 ; CC1(C)c2cc3cc(-c4nc(-c5ccccc5)nc(-c5ccccc5)n4)ccc3cc2-c2cc3sc(-c4ccc5ccccc5c4)nc3cc21]

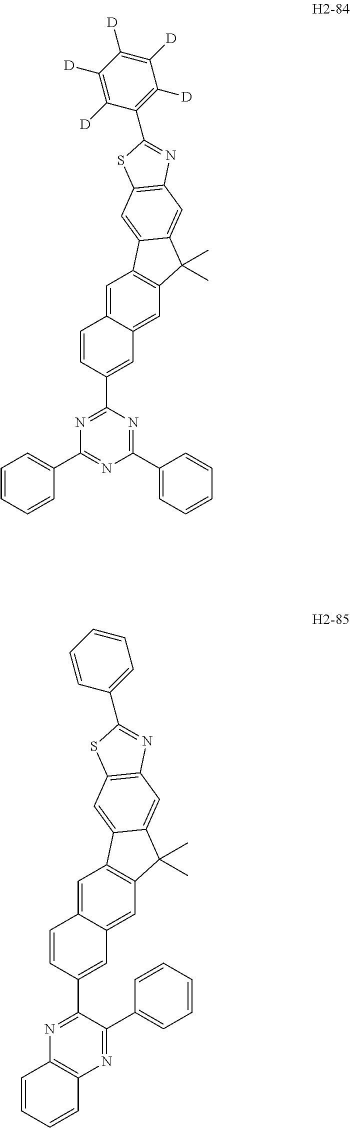 [SMILES: [2H]c1c([2H])c([2H])c(-c2nc3cc4c(cc3s2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3sc(-c4ccccc4)nc3cc21]

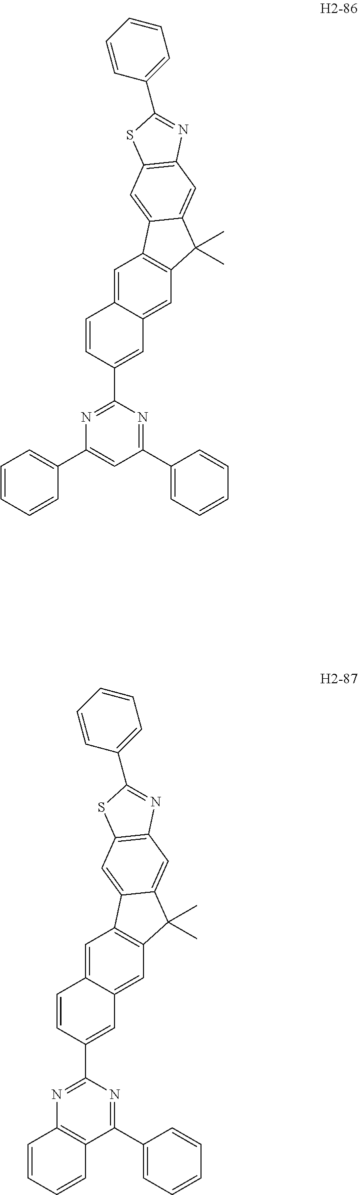 [SMILES: CC1(C)c2cc3cc(-c4nc(-c5ccccc5)cc(-c5ccccc5)n4)ccc3cc2-c2cc3sc(-c4ccccc4)nc3cc21 ; CC1(C)c2cc3cc(-c4nc(-c5ccccc5)c5ccccc5n4)ccc3cc2-c2cc3sc(-c4ccccc4)nc3cc21]

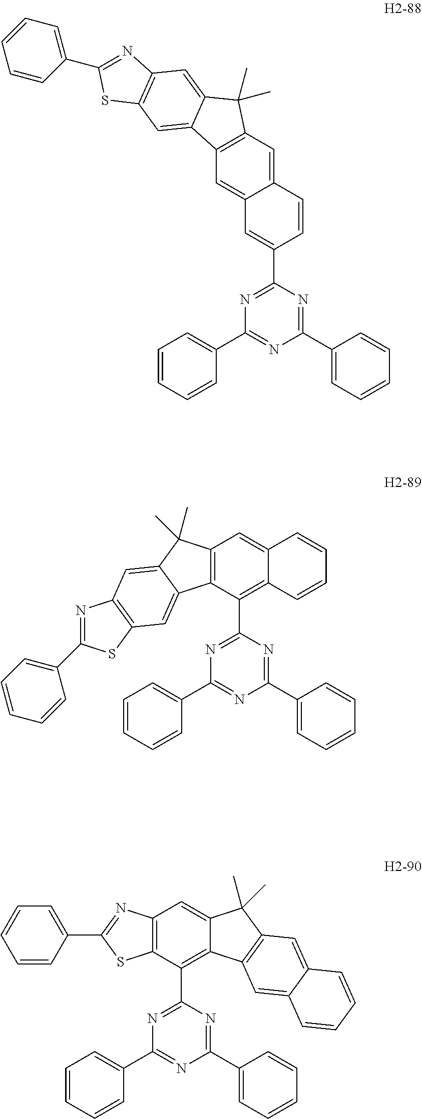 [SMILES: CC1(C)c2cc3ccc(-c4nc(-c5ccccc5)nc(-c5ccccc5)n4)cc3cc2-c2cc3sc(-c4ccccc4)nc3cc21 ; CC1(C)c2cc3nc(-c4ccccc4)sc3cc2-c2c1cc1ccccc1c2-c1nc(-c2ccccc2)nc(-c2ccccc2)n1 ; CC1(C)c2cc3ccccc3cc2-c2c1cc1nc(-c3ccccc3)sc1c2-c1nc(-c2ccccc2)nc(-c2ccccc2)n1]

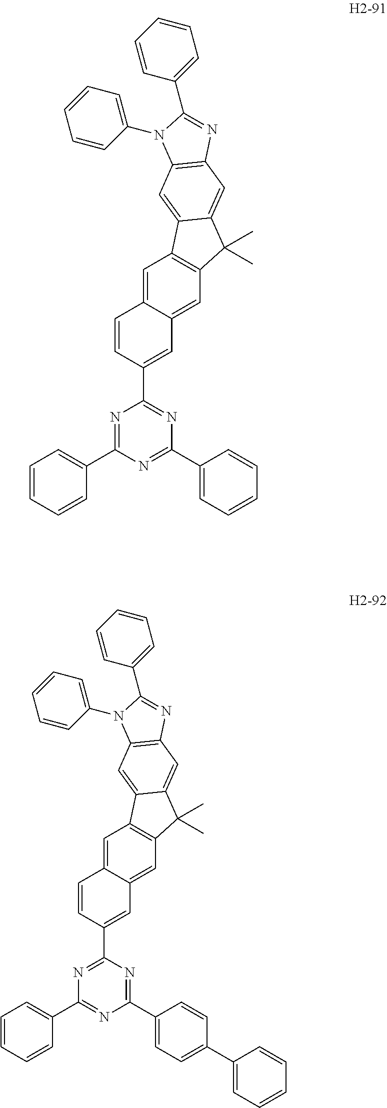 [SMILES: CC1(C)c2cc3cc(-c4nc(-c5ccccc5)nc(-c5ccccc5)n4)ccc3cc2-c2cc3c(cc21)nc(-c1ccccc1)n3-c1ccccc1 ; CC1(C)c2cc3cc(-c4nc(-c5ccccc5)nc(-c5ccc(-c6ccccc6)cc5)n4)ccc3cc2-c2cc3c(cc21)nc(-c1ccccc1)n3-c1ccccc1]

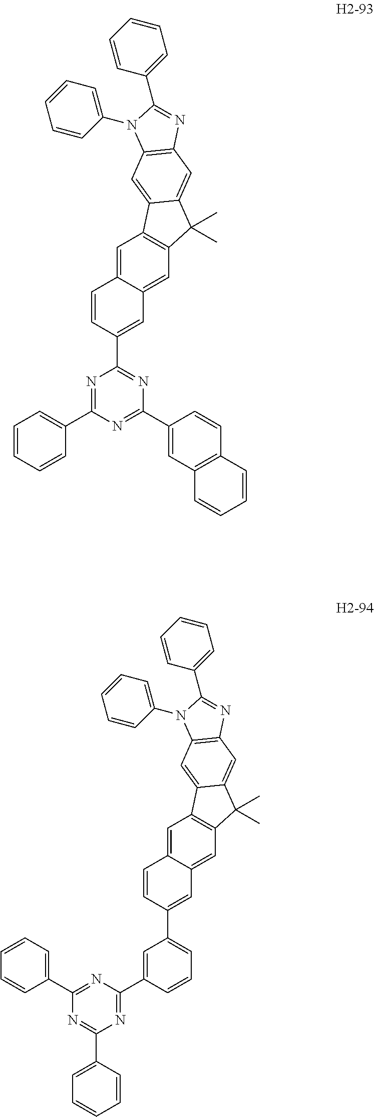 [SMILES: CC1(C)c2cc3cc(-c4nc(-c5ccccc5)nc(-c5ccc6ccccc6c5)n4)ccc3cc2-c2cc3c(cc21)nc(-c1ccccc1)n3-c1ccccc1 ; CC1(C)c2cc3cc(-c4cccc(-c5nc(-c6ccccc6)nc(-c6ccccc6)n5)c4)ccc3cc2-c2cc3c(cc21)nc(-c1ccccc1)n3-c1ccccc1]

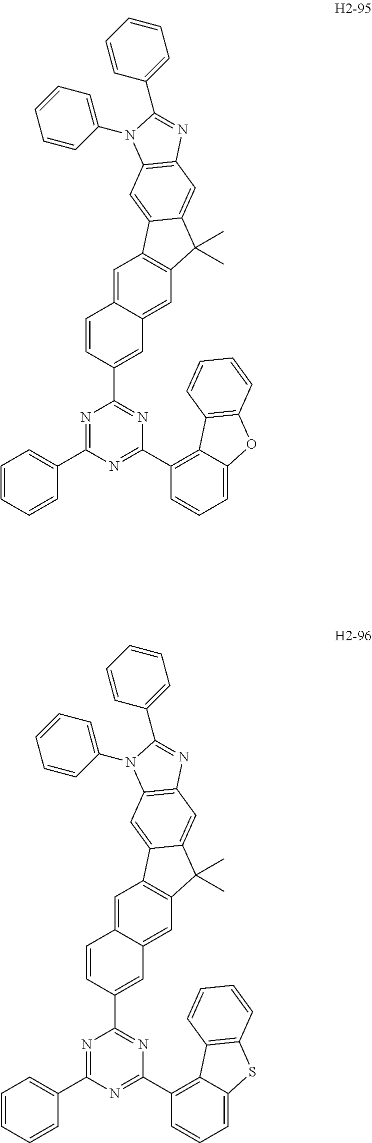 [SMILES: CC1(C)c2cc3cc(-c4nc(-c5ccccc5)nc(-c5cccc6oc7ccccc7c56)n4)ccc3cc2-c2cc3c(cc21)nc(-c1ccccc1)n3-c1ccccc1 ; CC1(C)c2cc3cc(-c4nc(-c5ccccc5)nc(-c5cccc6sc7ccccc7c56)n4)ccc3cc2-c2cc3c(cc21)nc(-c1ccccc1)n3-c1ccccc1]

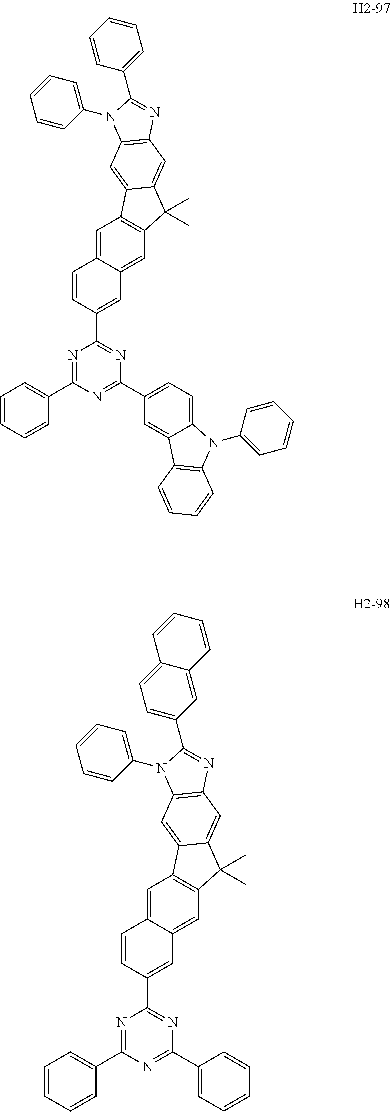 [SMILES: CC1(C)c2cc3cc(-c4nc(-c5ccccc5)nc(-c5ccc6c(c5)c5ccccc5n6-c5ccccc5)n4)ccc3cc2-c2cc3c(cc21)nc(-c1ccccc1)n3-c1ccccc1 ; CC1(C)c2cc3cc(-c4nc(-c5ccccc5)nc(-c5ccccc5)n4)ccc3cc2-c2cc3c(cc21)nc(-c1ccc2ccccc2c1)n3-c1ccccc1]

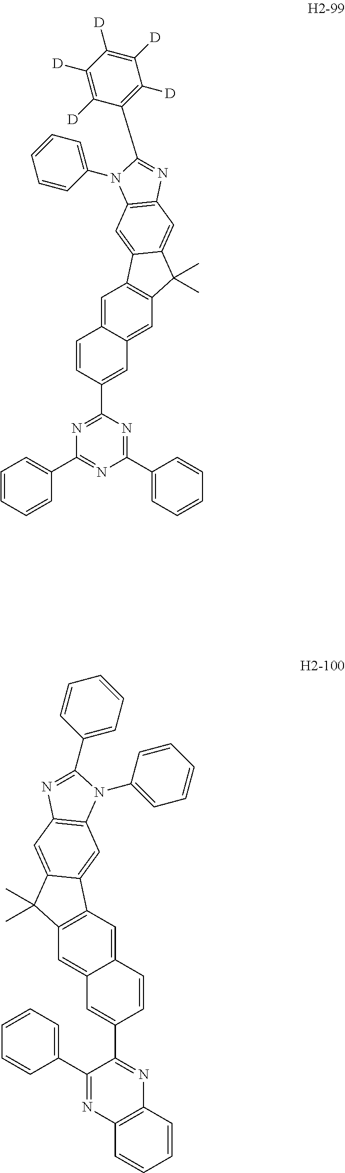 [SMILES: [2H]c1c([2H])c([2H])c(-c2nc3cc4c(cc3n2-c2ccccc2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3c(cc21)nc(-c1ccccc1)n3-c1ccccc1]

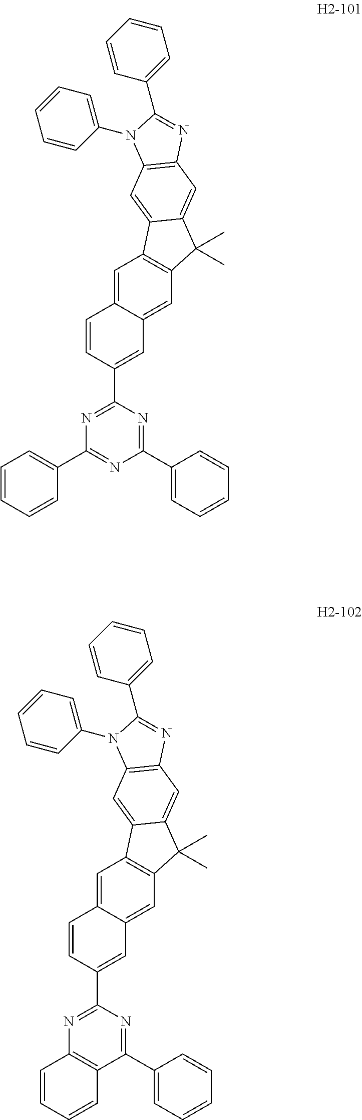 [SMILES: CC1(C)c2cc3cc(-c4nc(-c5ccccc5)nc(-c5ccccc5)n4)ccc3cc2-c2cc3c(cc21)nc(-c1ccccc1)n3-c1ccccc1 ; CC1(C)c2cc3cc(-c4nc(-c5ccccc5)c5ccccc5n4)ccc3cc2-c2cc3c(cc21)nc(-c1ccccc1)n3-c1ccccc1]

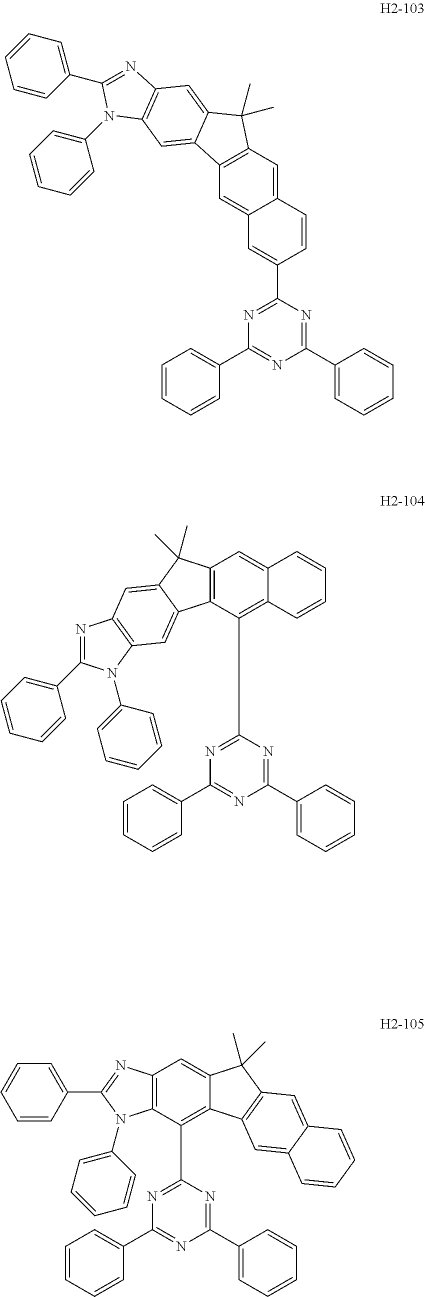 [SMILES: CC1(C)c2cc3ccc(-c4nc(-c5ccccc5)nc(-c5ccccc5)n4)cc3cc2-c2cc3c(cc21)nc(-c1ccccc1)n3-c1ccccc1 ; CC1(C)c2cc3nc(-c4ccccc4)n(-c4ccccc4)c3cc2-c2c1cc1ccccc1c2-c1nc(-c2ccccc2)nc(-c2ccccc2)n1 ; CC1(C)c2cc3ccccc3cc2-c2c1cc1nc(-c3ccccc3)n(-c3ccccc3)c1c2-c1nc(-c2ccccc2)nc(-c2ccccc2)n1]

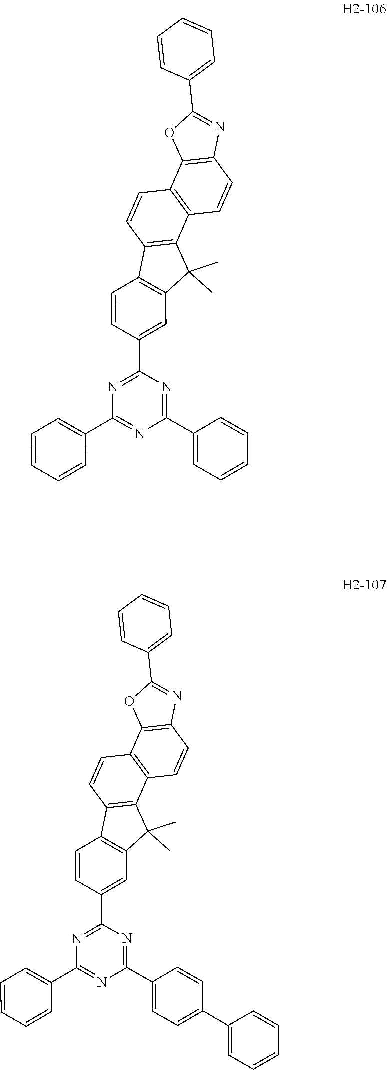 [SMILES: CC1(C)c2cc(-c3nc(-c4ccccc4)nc(-c4ccccc4)n3)ccc2-c2ccc3c(ccc4nc(-c5ccccc5)oc43)c21 ; CC1(C)c2cc(-c3nc(-c4ccccc4)nc(-c4ccc(-c5ccccc5)cc4)n3)ccc2-c2ccc3c(ccc4nc(-c5ccccc5)oc43)c21]

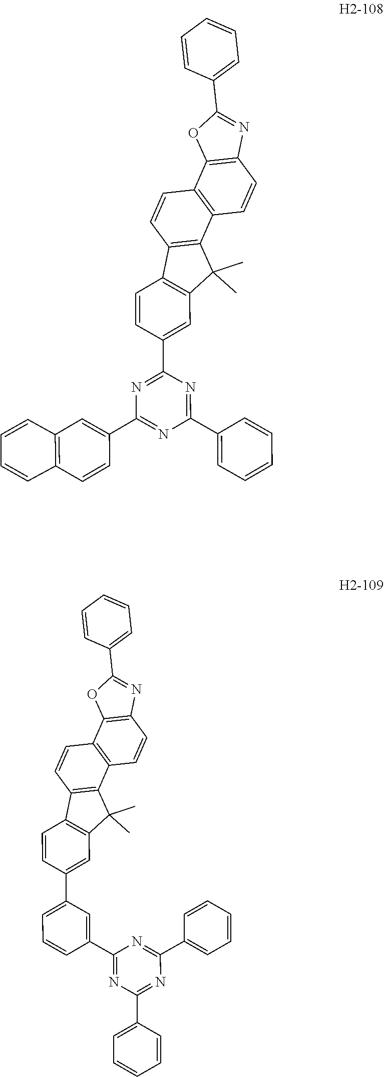 [SMILES: CC1(C)c2cc(-c3nc(-c4ccccc4)nc(-c4ccc5ccccc5c4)n3)ccc2-c2ccc3c(ccc4nc(-c5ccccc5)oc43)c21 ; CC1(C)c2cc(-c3cccc(-c4nc(-c5ccccc5)nc(-c5ccccc5)n4)c3)ccc2-c2ccc3c(ccc4nc(-c5ccccc5)oc43)c21]

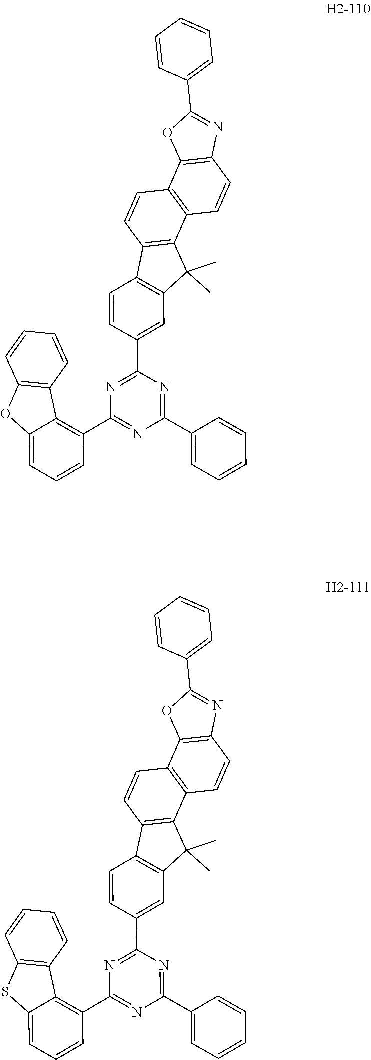 [SMILES: CC1(C)c2cc(-c3nc(-c4ccccc4)nc(-c4cccc5oc6ccccc6c45)n3)ccc2-c2ccc3c(ccc4nc(-c5ccccc5)oc43)c21 ; CC1(C)c2cc(-c3nc(-c4ccccc4)nc(-c4cccc5sc6ccccc6c45)n3)ccc2-c2ccc3c(ccc4nc(-c5ccccc5)oc43)c21]

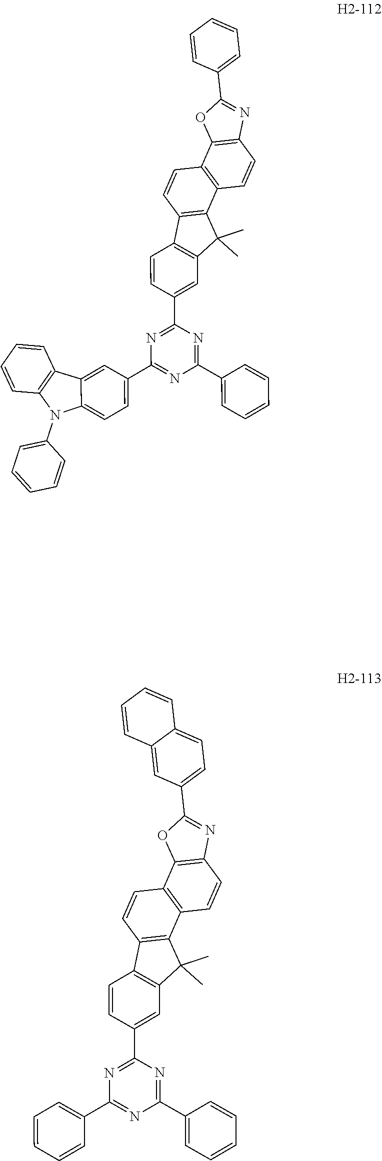 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)oc43)c21 ; CC1(C)c2cc(-c3nc(-c4ccccc4)nc(-c4ccccc4)n3)ccc2-c2ccc3c(ccc4nc(-c5ccc6ccccc6c5)oc43)c21]

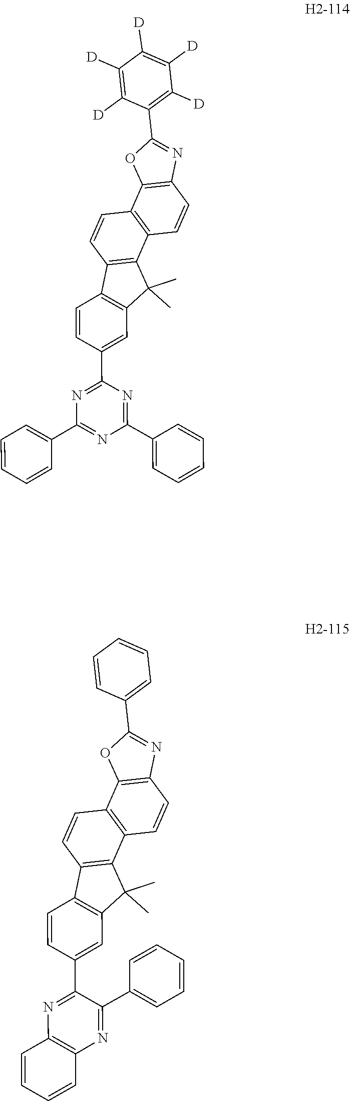 [SMILES: [2H]c1c([2H])c([2H])c(-c2nc3ccc4c5c(ccc4c3o2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)oc43)c21]

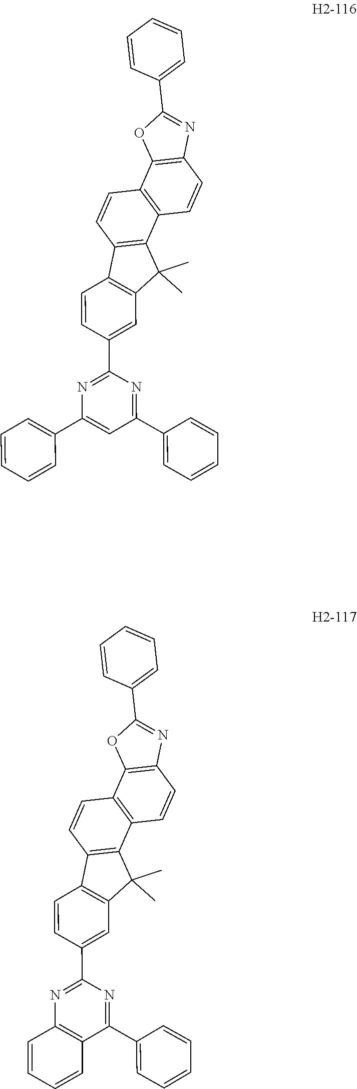 [SMILES: CC1(C)c2cc(-c3nc(-c4ccccc4)cc(-c4ccccc4)n3)ccc2-c2ccc3c(ccc4nc(-c5ccccc5)oc43)c21 ; CC1(C)c2cc(-c3nc(-c4ccccc4)c4ccccc4n3)ccc2-c2ccc3c(ccc4nc(-c5ccccc5)oc43)c21]

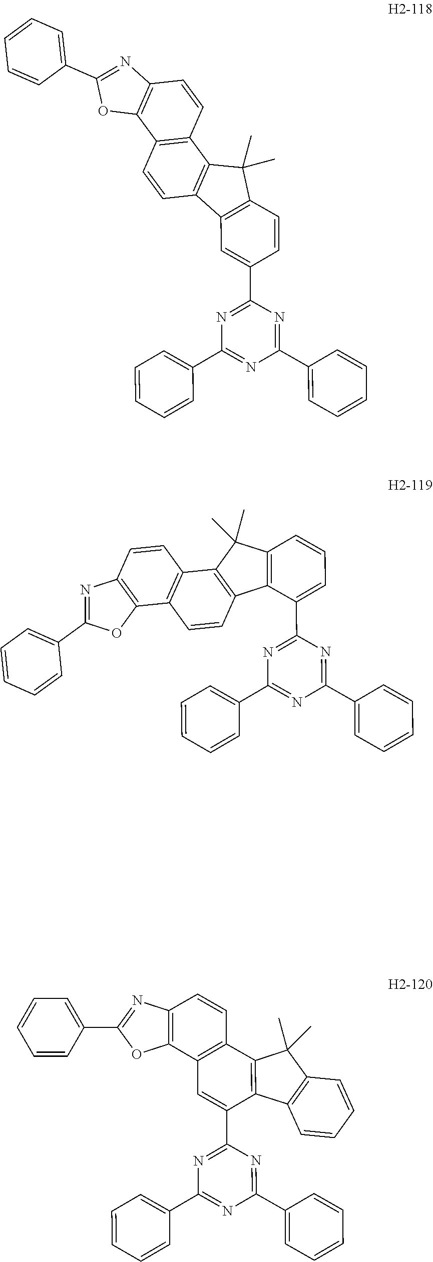 [SMILES: CC1(C)c2ccc(-c3nc(-c4ccccc4)nc(-c4ccccc4)n3)cc2-c2ccc3c(ccc4nc(-c5ccccc5)oc43)c21 ; CC1(C)c2cccc(-c3nc(-c4ccccc4)nc(-c4ccccc4)n3)c2-c2ccc3c(ccc4nc(-c5ccccc5)oc43)c21 ; CC1(C)c2ccccc2-c2c(-c3nc(-c4ccccc4)nc(-c4ccccc4)n3)cc3c(ccc4nc(-c5ccccc5)oc43)c21]

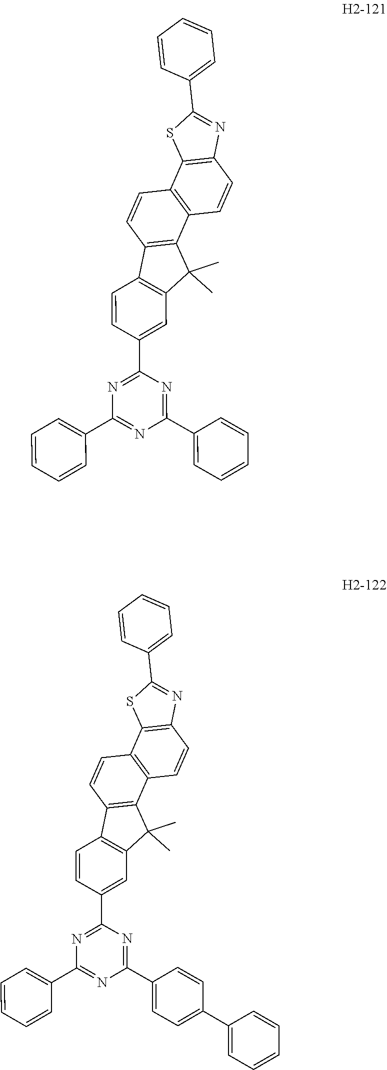 [SMILES: CC1(C)c2cc(-c3nc(-c4ccccc4)nc(-c4ccccc4)n3)ccc2-c2ccc3c(ccc4nc(-c5ccccc5)sc43)c21 ; CC1(C)c2cc(-c3nc(-c4ccccc4)nc(-c4ccc(-c5ccccc5)cc4)n3)ccc2-c2ccc3c(ccc4nc(-c5ccccc5)sc43)c21]

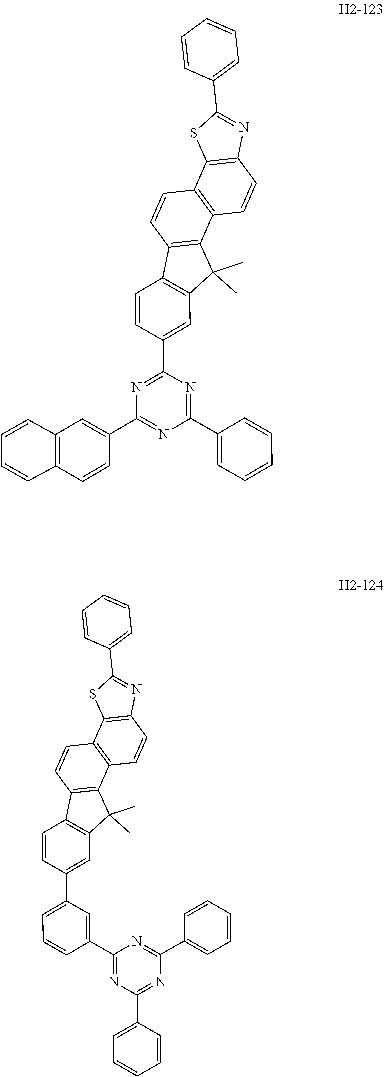 [SMILES: CC1(C)c2cc(-c3nc(-c4ccccc4)nc(-c4ccc5ccccc5c4)n3)ccc2-c2ccc3c(ccc4nc(-c5ccccc5)sc43)c21 ; CC1(C)c2cc(-c3cccc(-c4nc(-c5ccccc5)nc(-c5ccccc5)n4)c3)ccc2-c2ccc3c(ccc4nc(-c5ccccc5)sc43)c21]

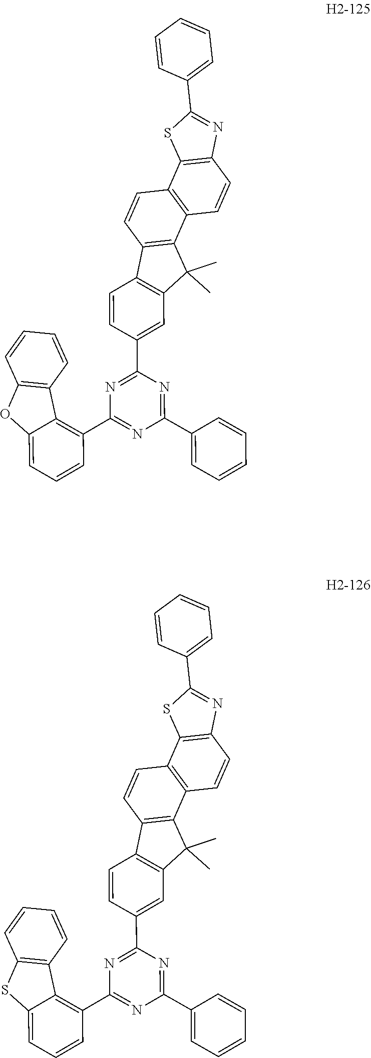 [SMILES: CC1(C)c2cc(-c3nc(-c4ccccc4)nc(-c4cccc5oc6ccccc6c45)n3)ccc2-c2ccc3c(ccc4nc(-c5ccccc5)sc43)c21 ; CC1(C)c2cc(-c3nc(-c4ccccc4)nc(-c4cccc5sc6ccccc6c45)n3)ccc2-c2ccc3c(ccc4nc(-c5ccccc5)sc43)c21]

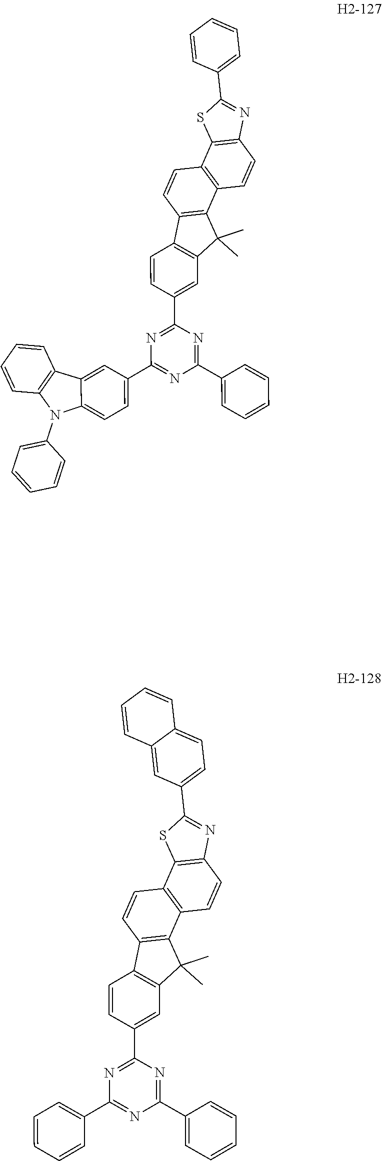 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)sc43)c21 ; CC1(C)c2cc(-c3nc(-c4ccccc4)nc(-c4ccccc4)n3)ccc2-c2ccc3c(ccc4nc(-c5ccc6ccccc6c5)sc43)c21]

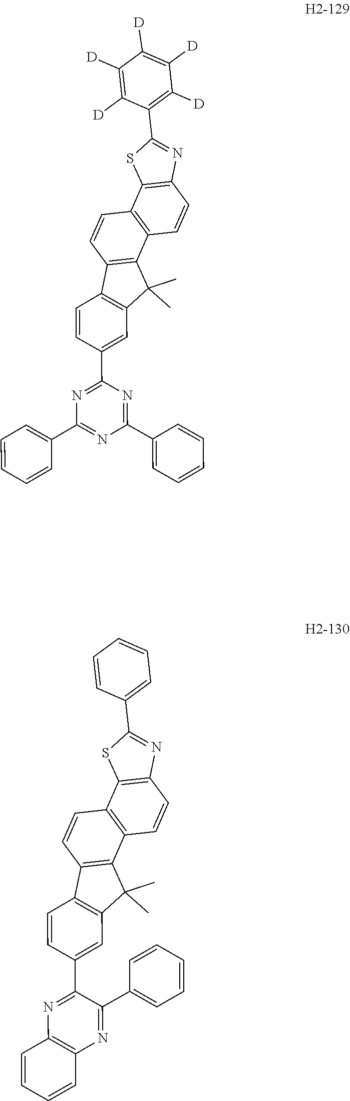 [SMILES: [2H]c1c([2H])c([2H])c(-c2nc3ccc4c5c(ccc4c3s2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)sc43)c21]

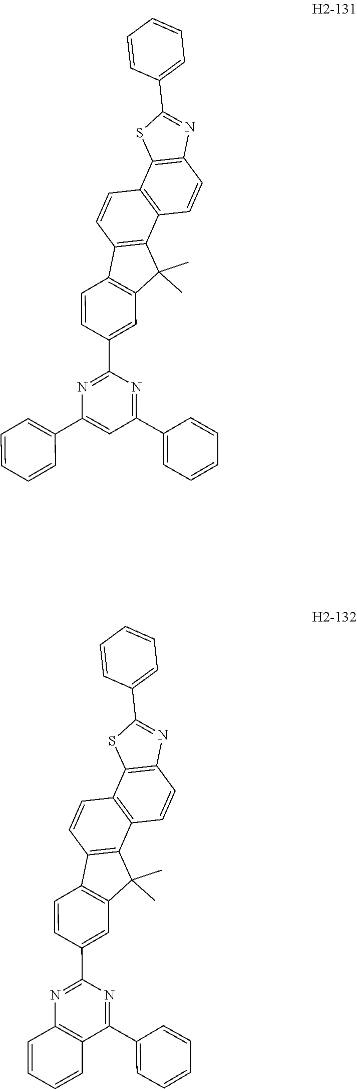 [SMILES: CC1(C)c2cc(-c3nc(-c4ccccc4)cc(-c4ccccc4)n3)ccc2-c2ccc3c(ccc4nc(-c5ccccc5)sc43)c21 ; CC1(C)c2cc(-c3nc(-c4ccccc4)c4ccccc4n3)ccc2-c2ccc3c(ccc4nc(-c5ccccc5)sc43)c21]

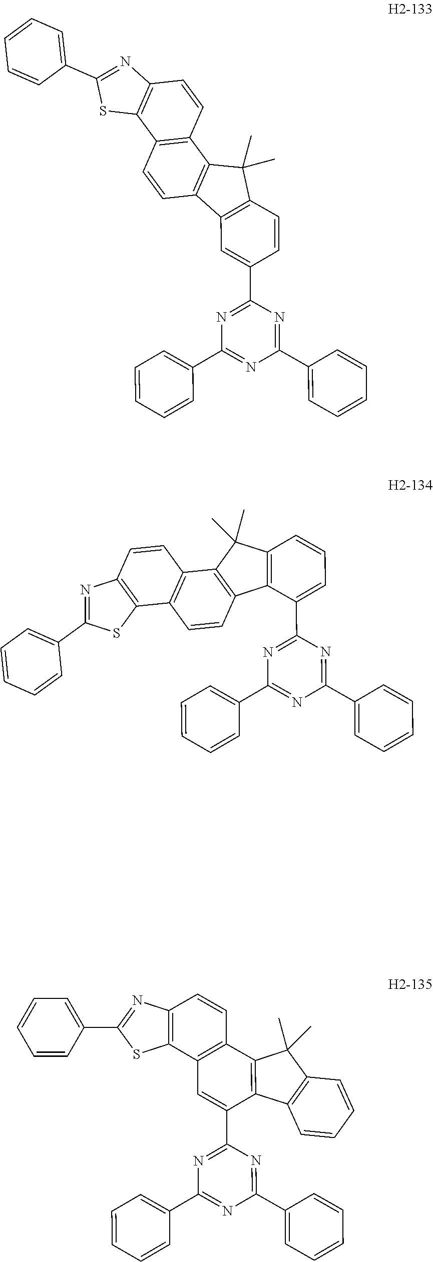 [SMILES: CC1(C)c2ccc(-c3nc(-c4ccccc4)nc(-c4ccccc4)n3)cc2-c2ccc3c(ccc4nc(-c5ccccc5)sc43)c21 ; CC1(C)c2cccc(-c3nc(-c4ccccc4)nc(-c4ccccc4)n3)c2-c2ccc3c(ccc4nc(-c5ccccc5)sc43)c21 ; CC1(C)c2ccccc2-c2c(-c3nc(-c4ccccc4)nc(-c4ccccc4)n3)cc3c(ccc4nc(-c5ccccc5)sc43)c21]

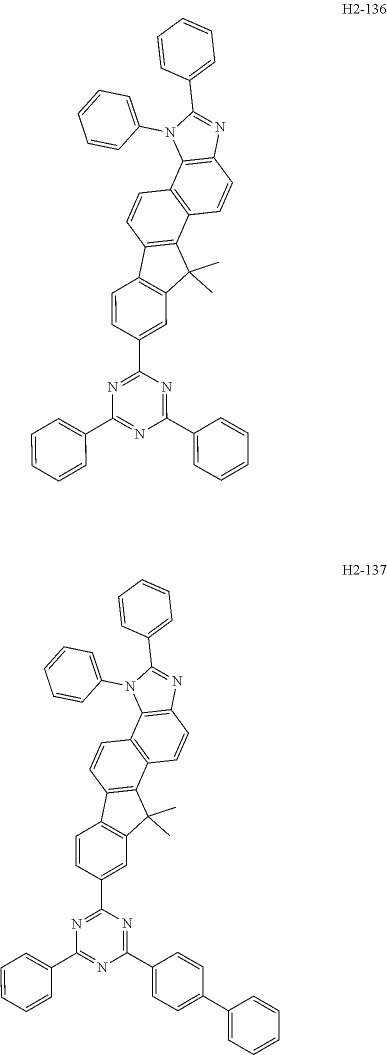 [SMILES: CC1(C)c2cc(-c3nc(-c4ccccc4)nc(-c4ccccc4)n3)ccc2-c2ccc3c(ccc4nc(-c5ccccc5)n(-c5ccccc5)c43)c21 ; CC1(C)c2cc(-c3nc(-c4ccccc4)nc(-c4ccc(-c5ccccc5)cc4)n3)ccc2-c2ccc3c(ccc4nc(-c5ccccc5)n(-c5ccccc5)c43)c21]

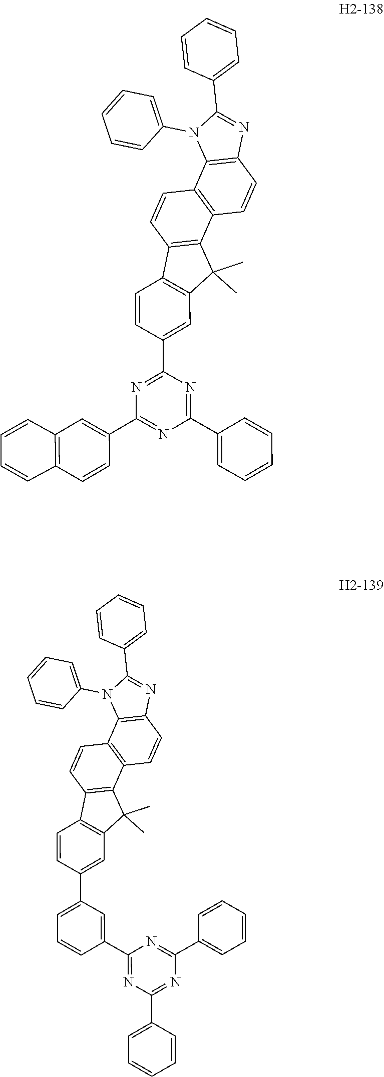 [SMILES: CC1(C)c2cc(-c3nc(-c4ccccc4)nc(-c4ccc5ccccc5c4)n3)ccc2-c2ccc3c(ccc4nc(-c5ccccc5)n(-c5ccccc5)c43)c21 ; CC1(C)c2cc(-c3cccc(-c4nc(-c5ccccc5)nc(-c5ccccc5)n4)c3)ccc2-c2ccc3c(ccc4nc(-c5ccccc5)n(-c5ccccc5)c43)c21]

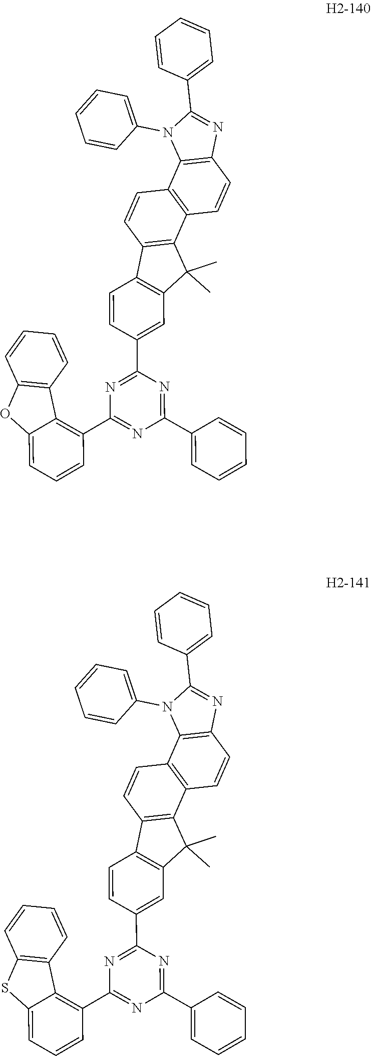 [SMILES: CC1(C)c2cc(-c3nc(-c4ccccc4)nc(-c4cccc5oc6ccccc6c45)n3)ccc2-c2ccc3c(ccc4nc(-c5ccccc5)n(-c5ccccc5)c43)c21 ; CC1(C)c2cc(-c3nc(-c4ccccc4)nc(-c4cccc5sc6ccccc6c45)n3)ccc2-c2ccc3c(ccc4nc(-c5ccccc5)n(-c5ccccc5)c43)c21]

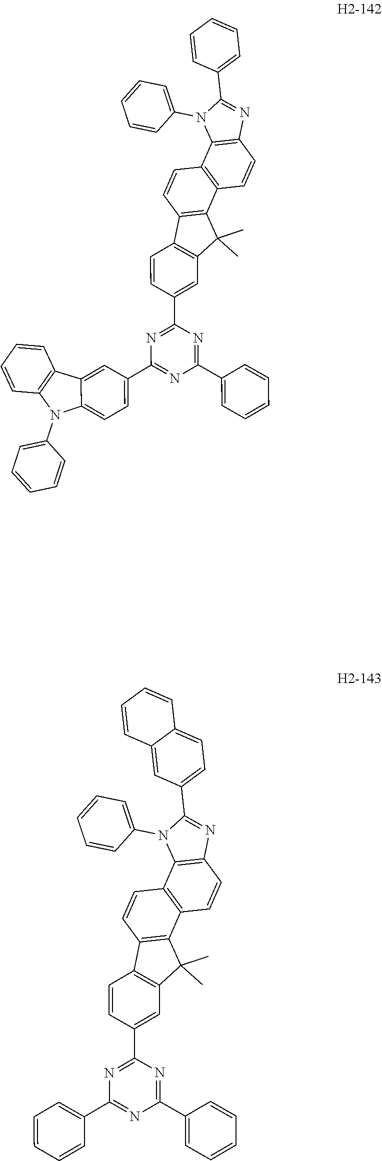 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)n(-c5ccccc5)c43)c21 ; CC1(C)c2cc(-c3nc(-c4ccccc4)nc(-c4ccccc4)n3)ccc2-c2ccc3c(ccc4nc(-c5ccc6ccccc6c5)n(-c5ccccc5)c43)c21]

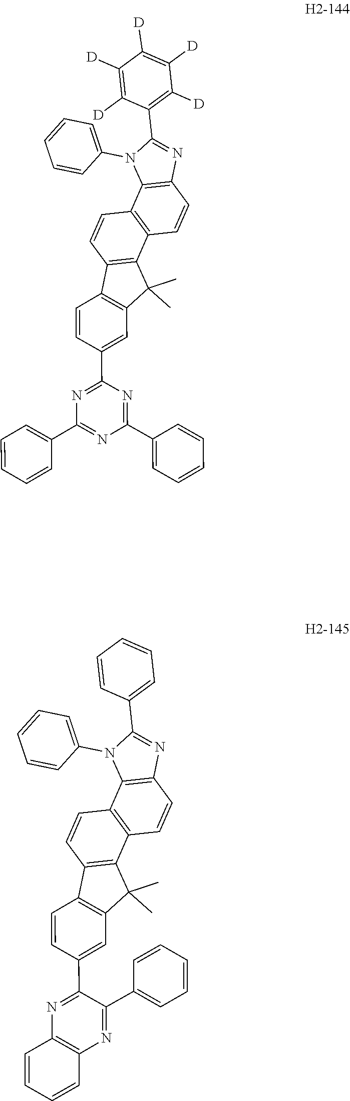 [SMILES: [2H]c1c([2H])c([2H])c(-c2nc3ccc4c5c(ccc4c3n2-c2ccccc2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)n(-c5ccccc5)c43)c21]

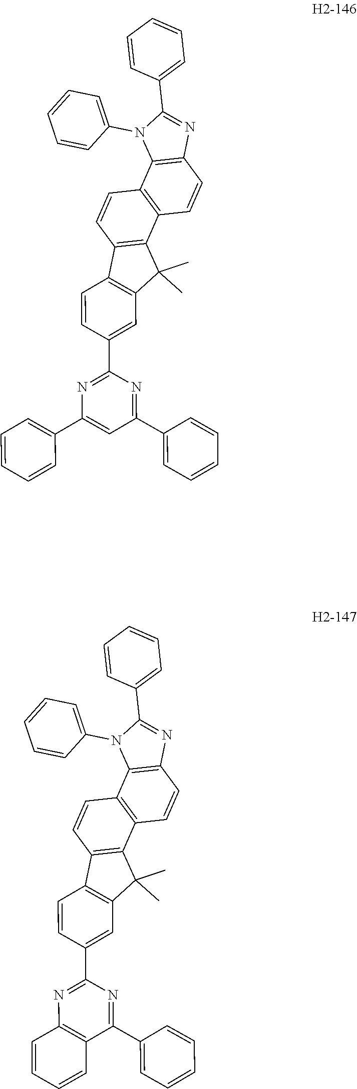 [SMILES: CC1(C)c2cc(-c3nc(-c4ccccc4)cc(-c4ccccc4)n3)ccc2-c2ccc3c(ccc4nc(-c5ccccc5)n(-c5ccccc5)c43)c21 ; CC1(C)c2cc(-c3nc(-c4ccccc4)c4ccccc4n3)ccc2-c2ccc3c(ccc4nc(-c5ccccc5)n(-c5ccccc5)c43)c21]

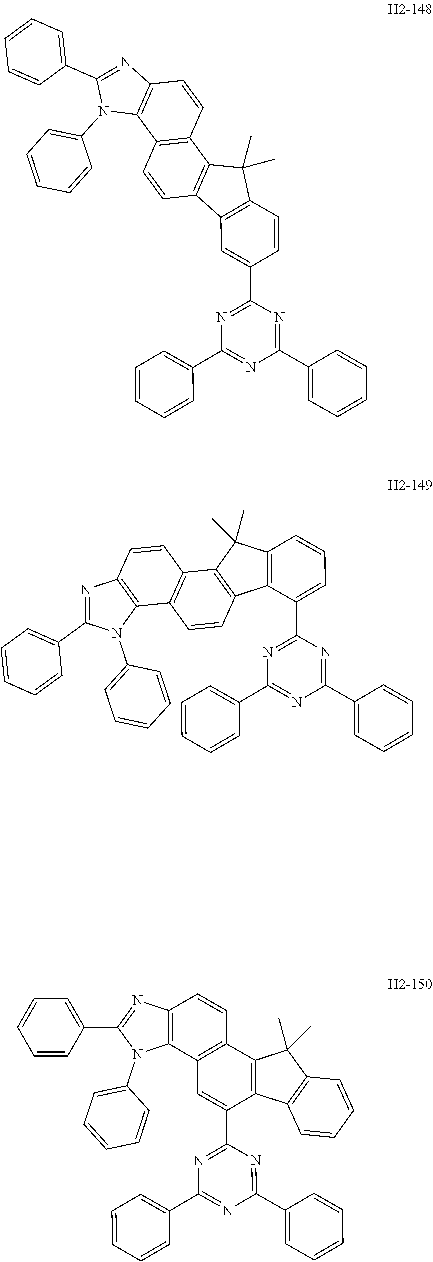 [SMILES: CC1(C)c2ccc(-c3nc(-c4ccccc4)nc(-c4ccccc4)n3)cc2-c2ccc3c(ccc4nc(-c5ccccc5)n(-c5ccccc5)c43)c21 ; CC1(C)c2cccc(-c3nc(-c4ccccc4)nc(-c4ccccc4)n3)c2-c2ccc3c(ccc4nc(-c5ccccc5)n(-c5ccccc5)c43)c21 ; CC1(C)c2ccccc2-c2c(-c3nc(-c4ccccc4)nc(-c4ccccc4)n3)cc3c(ccc4nc(-c5ccccc5)n(-c5ccccc5)c43)c21]

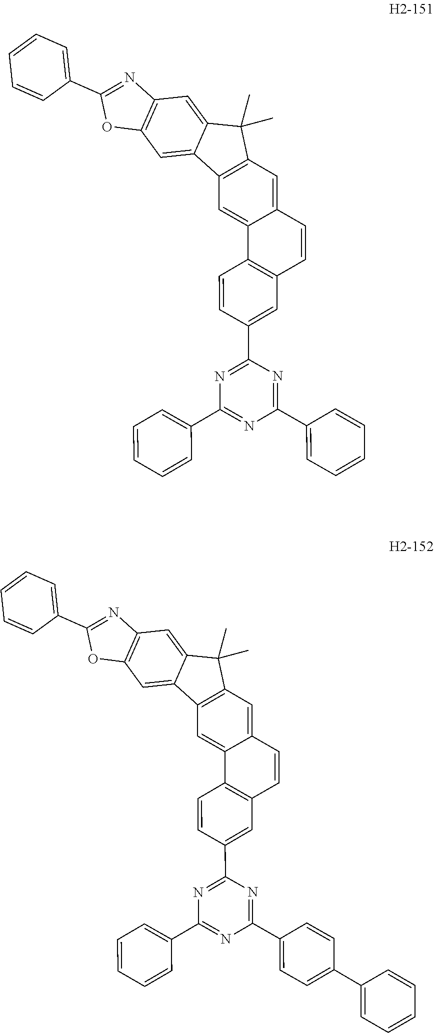 [SMILES: CC1(C)c2cc3ccc4cc(-c5nc(-c6ccccc6)nc(-c6ccccc6)n5)ccc4c3cc2-c2cc3oc(-c4ccccc4)nc3cc21 ; CC1(C)c2cc3ccc4cc(-c5nc(-c6ccccc6)nc(-c6ccc(-c7ccccc7)cc6)n5)ccc4c3cc2-c2cc3oc(-c4ccccc4)nc3cc21]

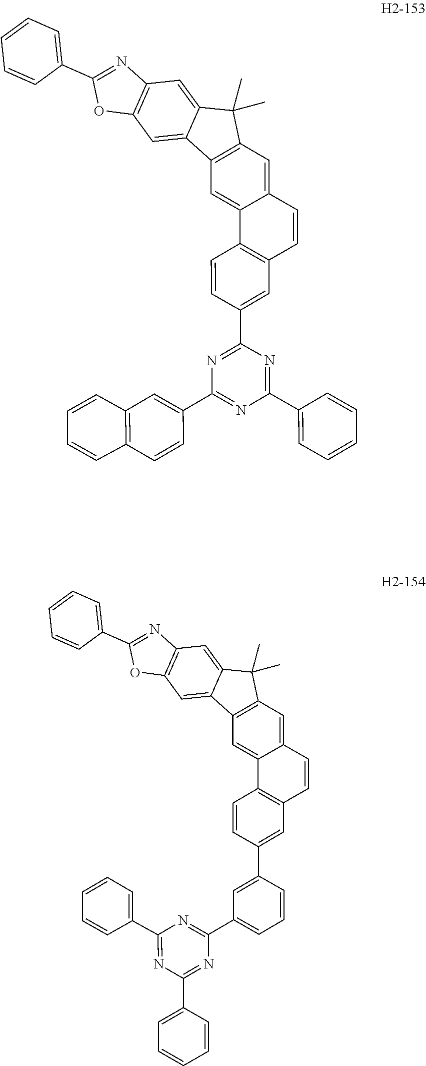 [SMILES: CC1(C)c2cc3ccc4cc(-c5nc(-c6ccccc6)nc(-c6ccc7ccccc7c6)n5)ccc4c3cc2-c2cc3oc(-c4ccccc4)nc3cc21 ; CC1(C)c2cc3ccc4cc(-c5cccc(-c6nc(-c7ccccc7)nc(-c7ccccc7)n6)c5)ccc4c3cc2-c2cc3oc(-c4ccccc4)nc3cc21]

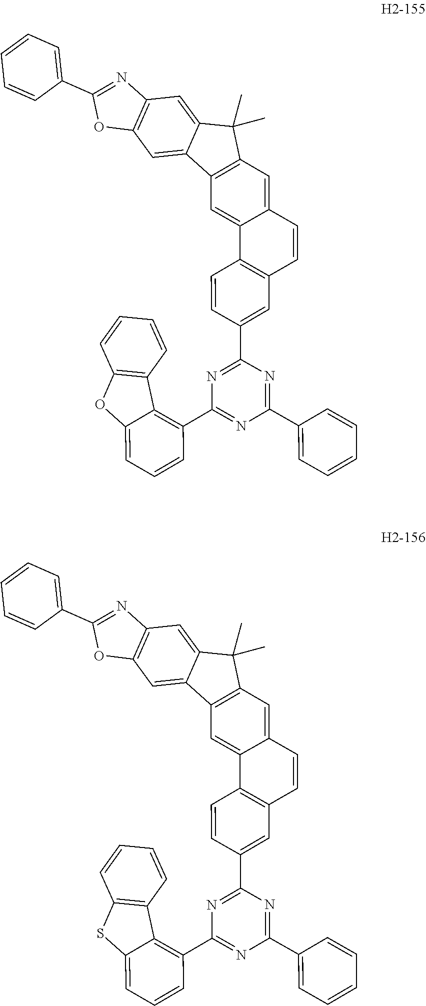 [SMILES: CC1(C)c2cc3ccc4cc(-c5nc(-c6ccccc6)nc(-c6cccc7oc8ccccc8c67)n5)ccc4c3cc2-c2cc3oc(-c4ccccc4)nc3cc21 ; CC1(C)c2cc3ccc4cc(-c5nc(-c6ccccc6)nc(-c6cccc7sc8ccccc8c67)n5)ccc4c3cc2-c2cc3oc(-c4ccccc4)nc3cc21]

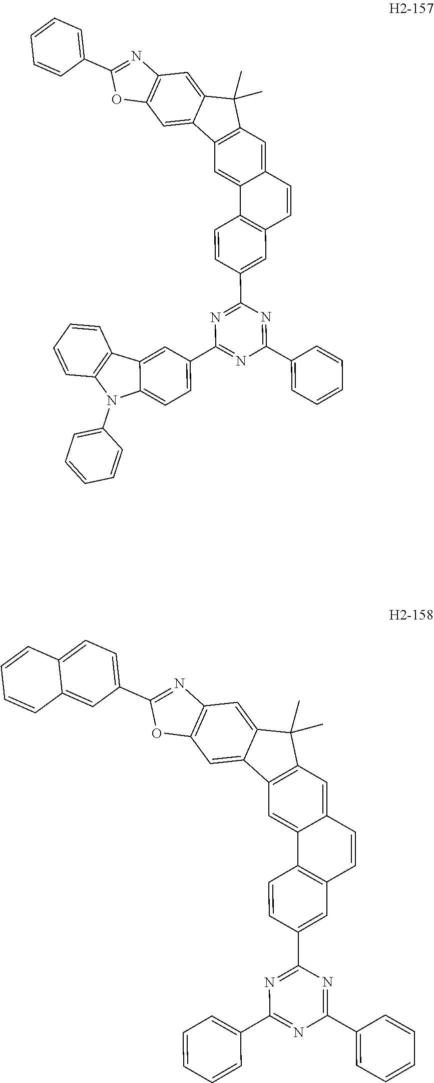 [SMILES: CC1(C)c2cc3ccc4cc(-c5nc(-c6ccccc6)nc(-c6ccc7c(c6)c6ccccc6n7-c6ccccc6)n5)ccc4c3cc2-c2cc3oc(-c4ccccc4)nc3cc21 ; CC1(C)c2cc3ccc4cc(-c5nc(-c6ccccc6)nc(-c6ccccc6)n5)ccc4c3cc2-c2cc3oc(-c4ccc5ccccc5c4)nc3cc21]

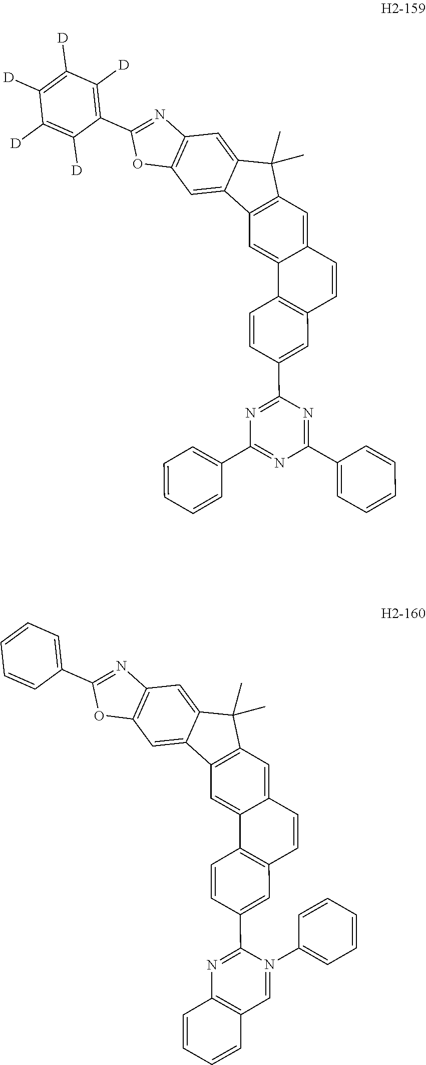

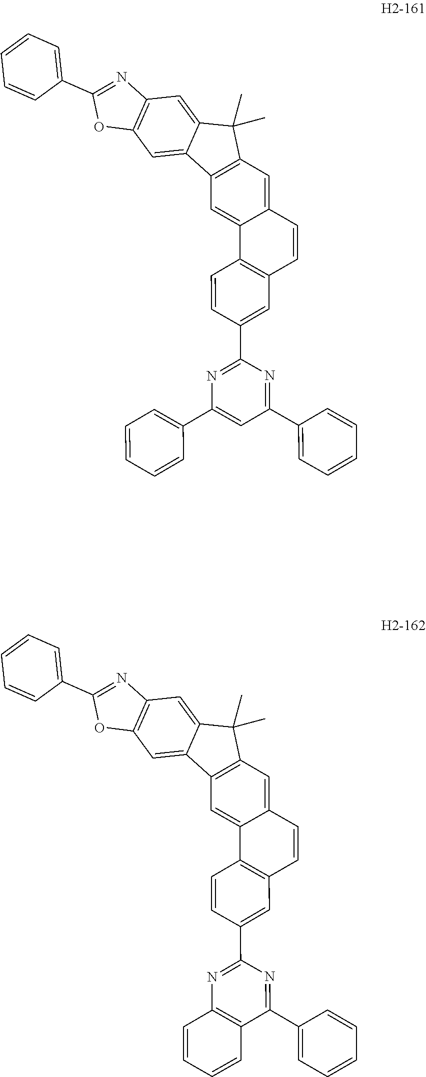 [SMILES: CC1(C)c2cc3ccc4cc(-c5nc(-c6ccccc6)cc(-c6ccccc6)n5)ccc4c3cc2-c2cc3oc(-c4ccccc4)nc3cc21 ; CC1(C)c2cc3ccc4cc(-c5nc(-c6ccccc6)c6ccccc6n5)ccc4c3cc2-c2cc3oc(-c4ccccc4)nc3cc21]

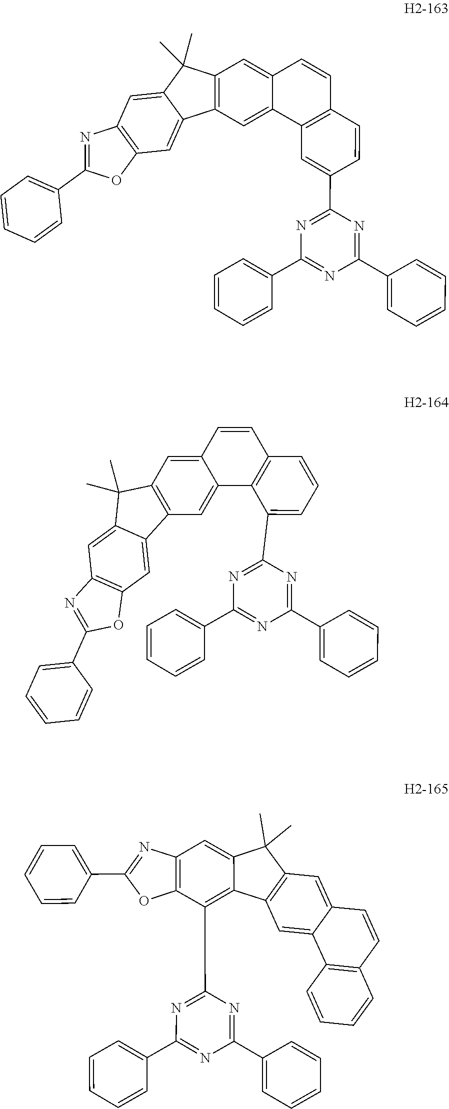 [SMILES: CC1(C)c2cc3ccc4ccc(-c5nc(-c6ccccc6)nc(-c6ccccc6)n5)cc4c3cc2-c2cc3oc(-c4ccccc4)nc3cc21 ; CC1(C)c2cc3ccc4cccc(-c5nc(-c6ccccc6)nc(-c6ccccc6)n5)c4c3cc2-c2cc3oc(-c4ccccc4)nc3cc21 ; CC1(C)c2cc3ccc4ccccc4c3cc2-c2c1cc1nc(-c3ccccc3)oc1c2-c1nc(-c2ccccc2)nc(-c2ccccc2)n1]

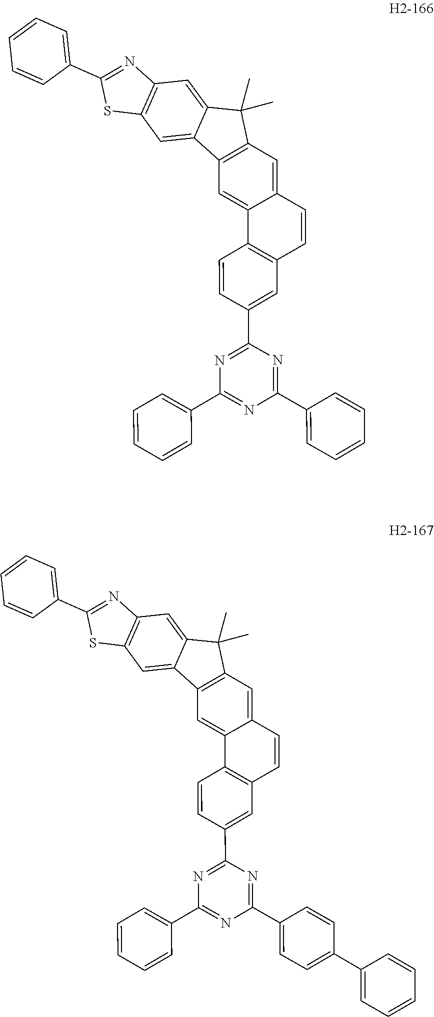 [SMILES: CC1(C)c2cc3ccc4cc(-c5nc(-c6ccccc6)nc(-c6ccccc6)n5)ccc4c3cc2-c2cc3sc(-c4ccccc4)nc3cc21 ; CC1(C)c2cc3ccc4cc(-c5nc(-c6ccccc6)nc(-c6ccc(-c7ccccc7)cc6)n5)ccc4c3cc2-c2cc3sc(-c4ccccc4)nc3cc21]

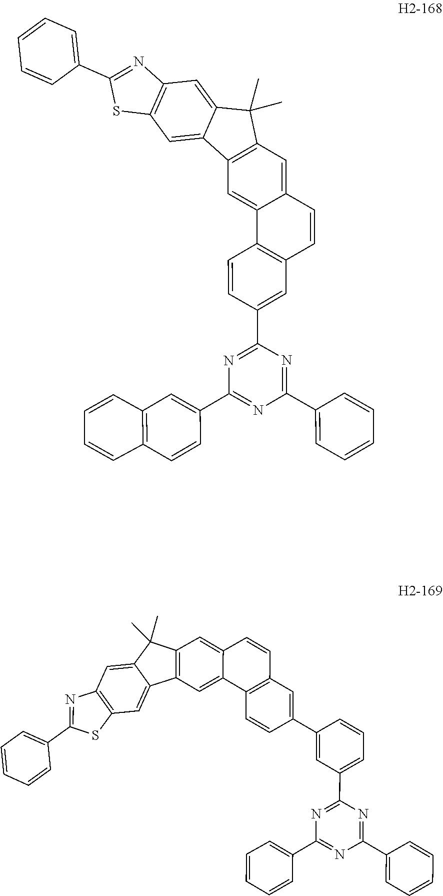 [SMILES: CC1(C)c2cc3ccc4cc(-c5nc(-c6ccccc6)nc(-c6ccc7ccccc7c6)n5)ccc4c3cc2-c2cc3sc(-c4ccccc4)nc3cc21 ; CC1(C)c2cc3ccc4cc(-c5cccc(-c6nc(-c7ccccc7)nc(-c7ccccc7)n6)c5)ccc4c3cc2-c2cc3sc(-c4ccccc4)nc3cc21]

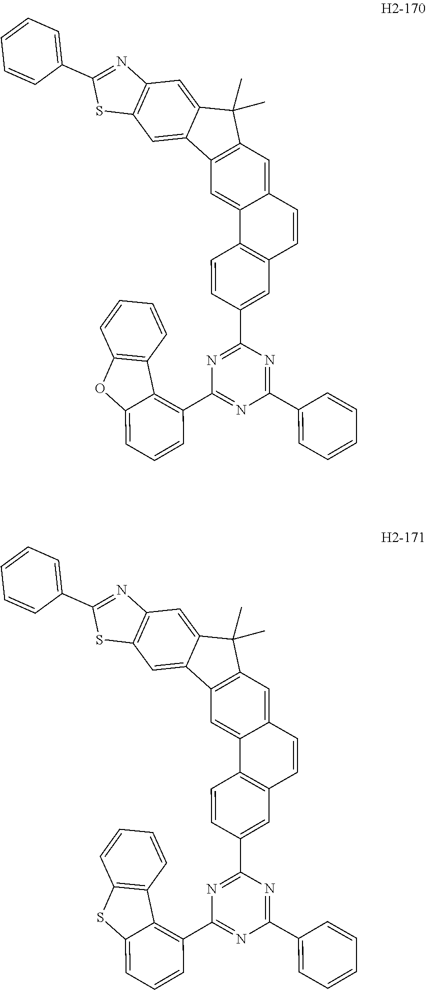 [SMILES: CC1(C)c2cc3ccc4cc(-c5nc(-c6ccccc6)nc(-c6cccc7oc8ccccc8c67)n5)ccc4c3cc2-c2cc3sc(-c4ccccc4)nc3cc21 ; CC1(C)c2cc3ccc4cc(-c5nc(-c6ccccc6)nc(-c6cccc7sc8ccccc8c67)n5)ccc4c3cc2-c2cc3sc(-c4ccccc4)nc3cc21]

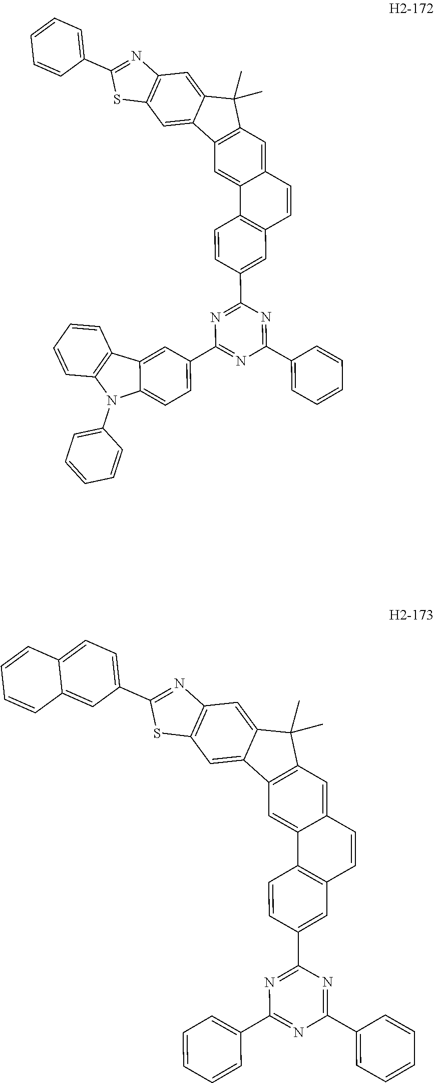 [SMILES: CC1(C)c2cc3ccc4cc(-c5nc(-c6ccccc6)nc(-c6ccc7c(c6)c6ccccc6n7-c6ccccc6)n5)ccc4c3cc2-c2cc3sc(-c4ccccc4)nc3cc21 ; CC1(C)c2cc3ccc4cc(-c5nc(-c6ccccc6)nc(-c6ccccc6)n5)ccc4c3cc2-c2cc3sc(-c4ccc5ccccc5c4)nc3cc21]

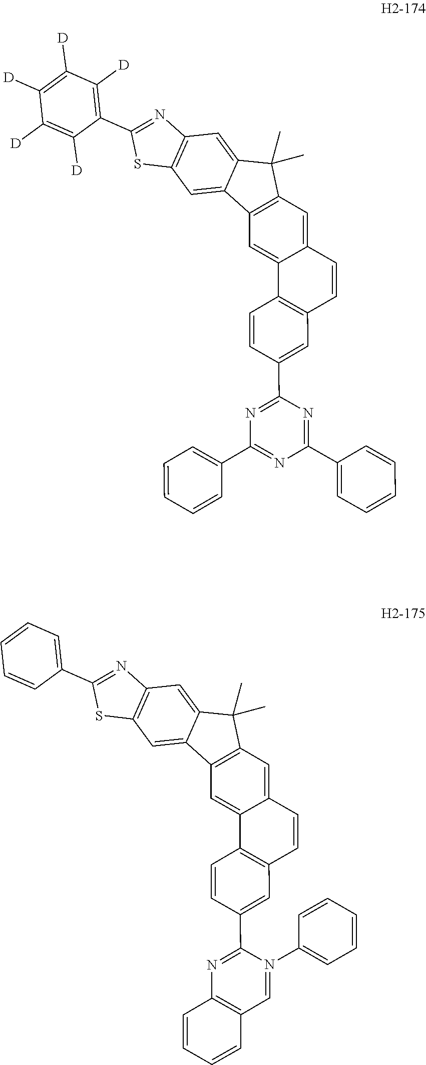

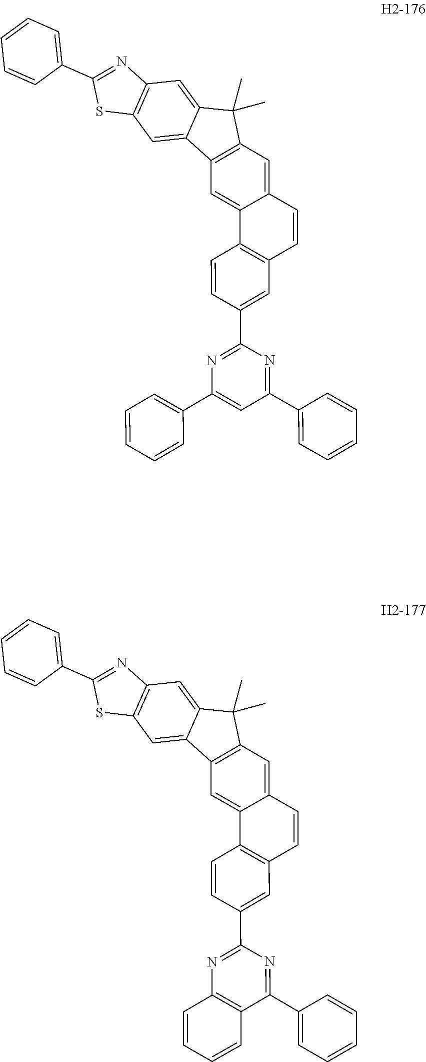 [SMILES: CC1(C)c2cc3ccc4cc(-c5nc(-c6ccccc6)cc(-c6ccccc6)n5)ccc4c3cc2-c2cc3sc(-c4ccccc4)nc3cc21 ; CC1(C)c2cc3ccc4cc(-c5nc(-c6ccccc6)c6ccccc6n5)ccc4c3cc2-c2cc3sc(-c4ccccc4)nc3cc21]

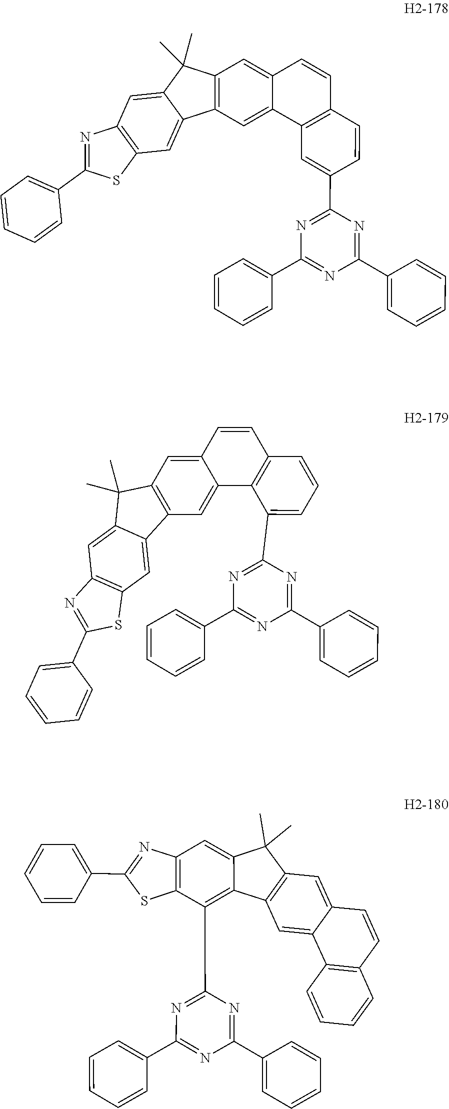 [SMILES: CC1(C)c2cc3ccc4ccc(-c5nc(-c6ccccc6)nc(-c6ccccc6)n5)cc4c3cc2-c2cc3sc(-c4ccccc4)nc3cc21 ; CC1(C)c2cc3ccc4cccc(-c5nc(-c6ccccc6)nc(-c6ccccc6)n5)c4c3cc2-c2cc3sc(-c4ccccc4)nc3cc21 ; CC1(C)c2cc3ccc4ccccc4c3cc2-c2c1cc1nc(-c3ccccc3)sc1c2-c1nc(-c2ccccc2)nc(-c2ccccc2)n1]

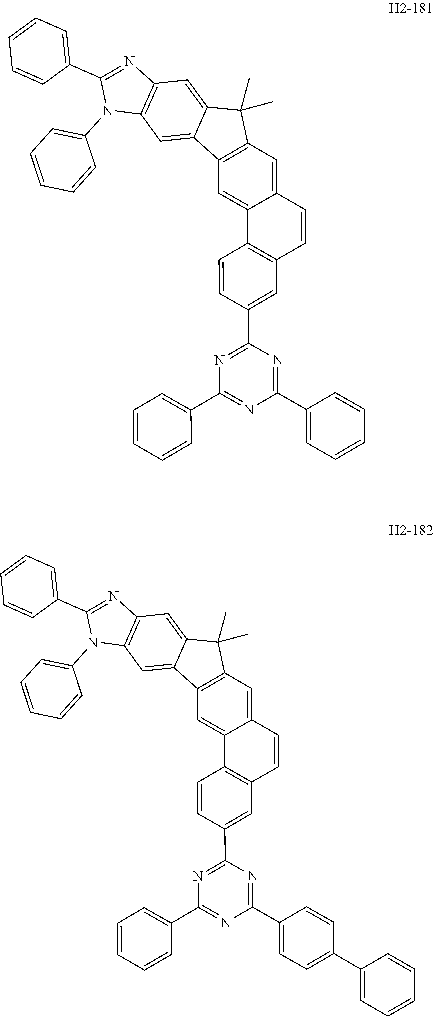 [SMILES: CC1(C)c2cc3ccc4cc(-c5nc(-c6ccccc6)nc(-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccc(-c7ccccc7)cc6)n5)ccc4c3cc2-c2cc3c(cc21)nc(-c1ccccc1)n3-c1ccccc1]

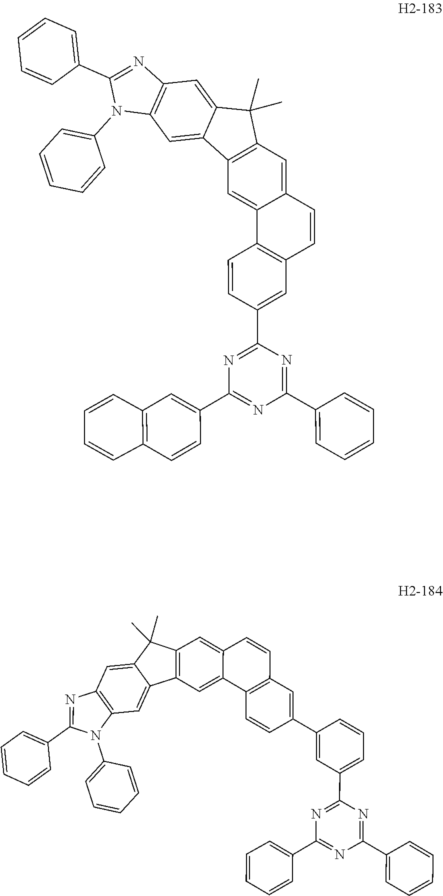 [SMILES: CC1(C)c2cc3ccc4cc(-c5nc(-c6ccccc6)nc(-c6ccc7ccccc7c6)n5)ccc4c3cc2-c2cc3c(cc21)nc(-c1ccccc1)n3-c1ccccc1 ; CC1(C)c2cc3ccc4cc(-c5cccc(-c6nc(-c7ccccc7)nc(-c7ccccc7)n6)c5)ccc4c3cc2-c2cc3c(cc21)nc(-c1ccccc1)n3-c1ccccc1]

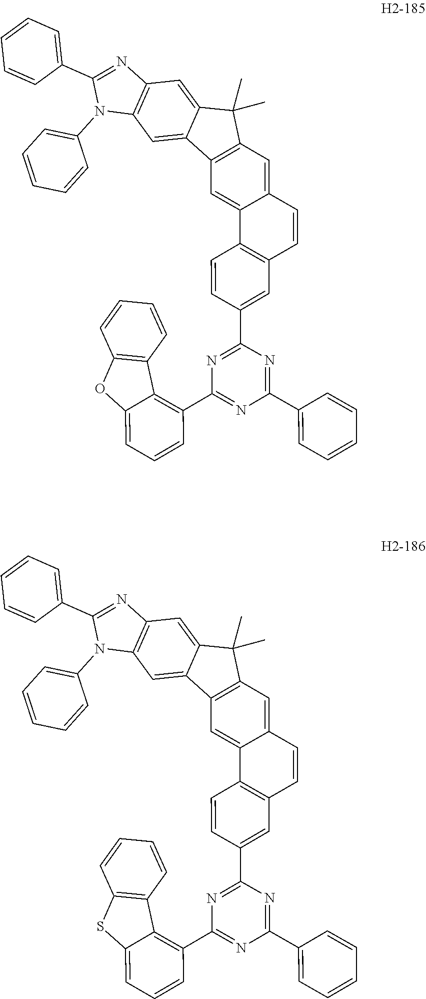 [SMILES: CC1(C)c2cc3ccc4cc(-c5nc(-c6ccccc6)nc(-c6cccc7oc8ccccc8c67)n5)ccc4c3cc2-c2cc3c(cc21)nc(-c1ccccc1)n3-c1ccccc1 ; CC1(C)c2cc3ccc4cc(-c5nc(-c6ccccc6)nc(-c6cccc7sc8ccccc8c67)n5)ccc4c3cc2-c2cc3c(cc21)nc(-c1ccccc1)n3-c1ccccc1]

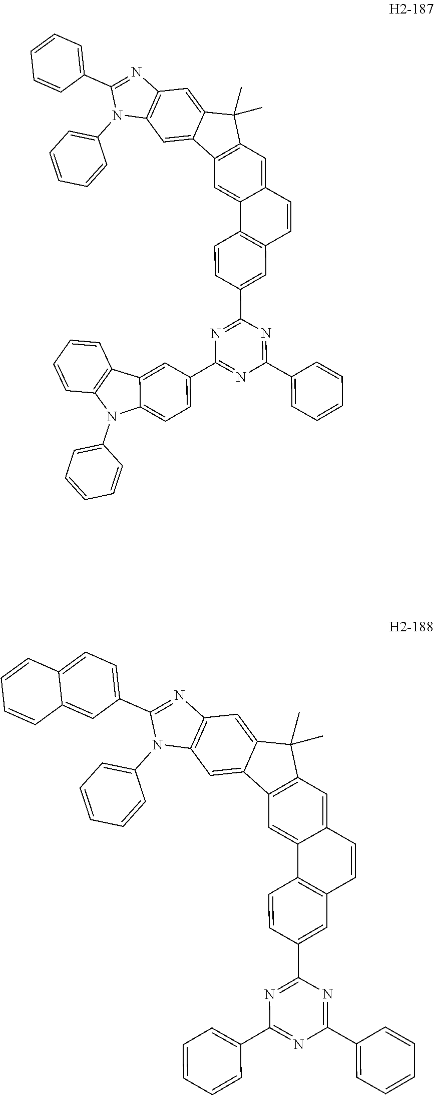 [SMILES: CC1(C)c2cc3ccc4cc(-c5nc(-c6ccccc6)nc(-c6ccc7c(c6)c6ccccc6n7-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccccc6)n5)ccc4c3cc2-c2cc3c(cc21)nc(-c1ccc2ccccc2c1)n3-c1ccccc1]

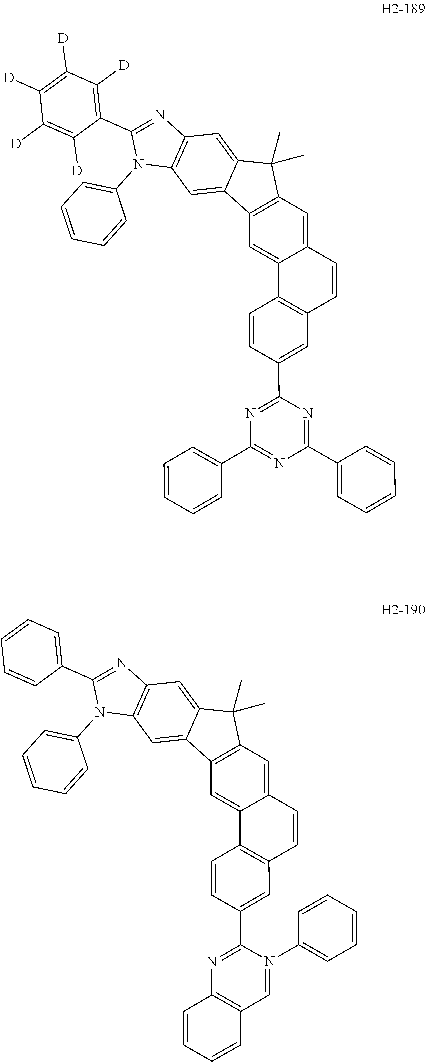

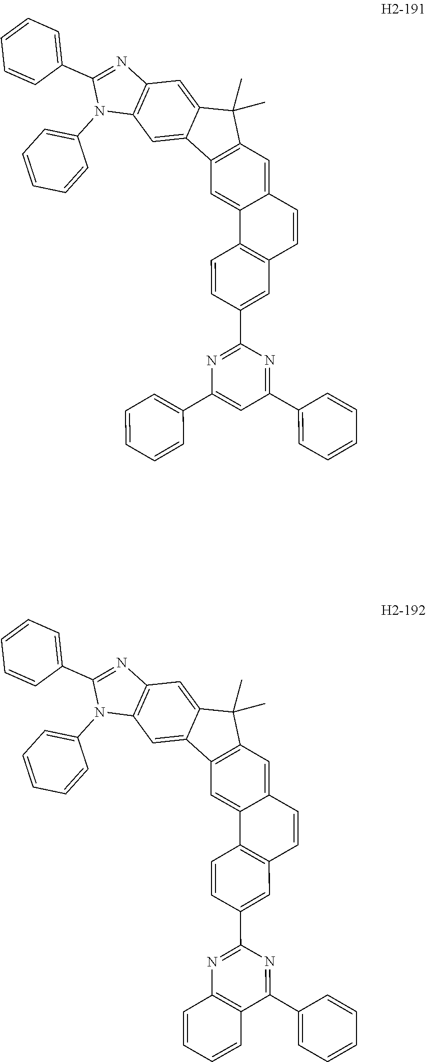 [SMILES: CC1(C)c2cc3ccc4cc(-c5nc(-c6ccccc6)cc(-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)c6ccccc6n5)ccc4c3cc2-c2cc3c(cc21)nc(-c1ccccc1)n3-c1ccccc1]

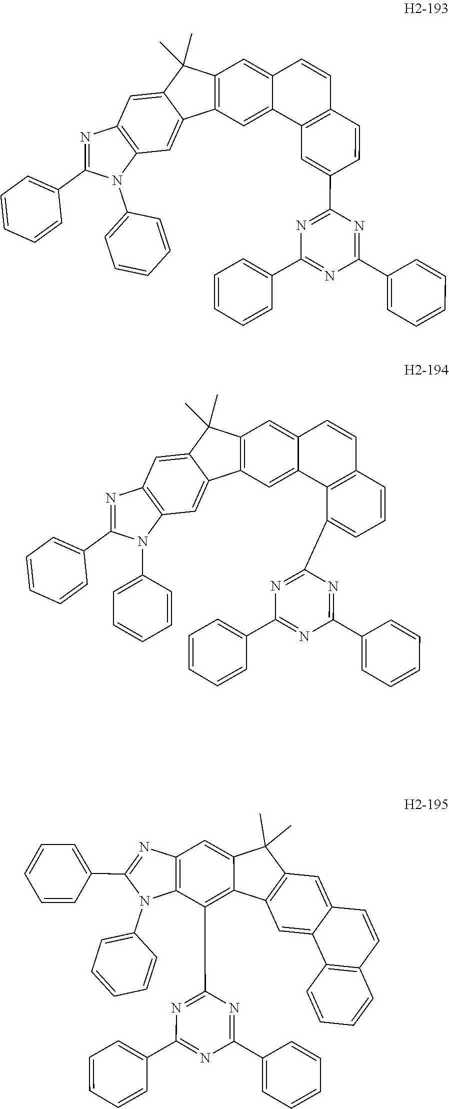 [SMILES: CC1(C)c2cc3ccc4ccc(-c5nc(-c6ccccc6)nc(-c6ccccc6)n5)cc4c3cc2-c2cc3c(cc21)nc(-c1ccccc1)n3-c1ccccc1 ; CC1(C)c2cc3ccc4cccc(-c5nc(-c6ccccc6)nc(-c6ccccc6)n5)c4c3cc2-c2cc3c(cc21)nc(-c1ccccc1)n3-c1ccccc1 ; CC1(C)c2cc3ccc4ccccc4c3cc2-c2c1cc1nc(-c3ccccc3)n(-c3ccccc3)c1c2-c1nc(-c2ccccc2)nc(-c2ccccc2)n1]

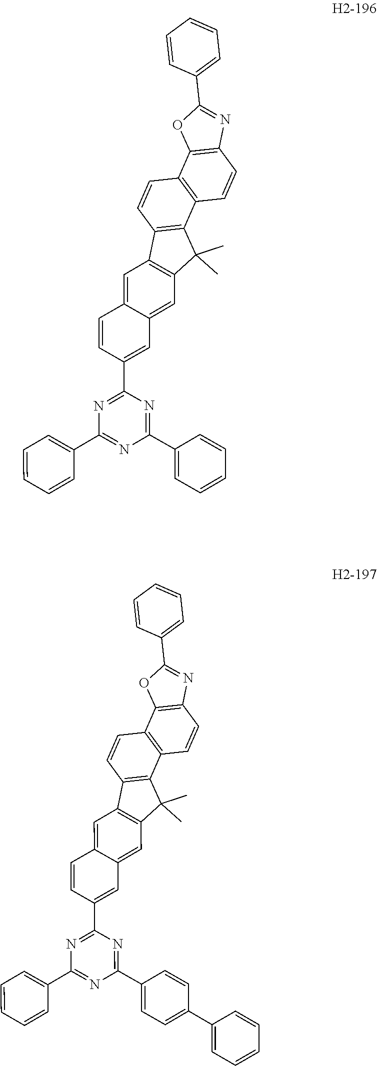 [SMILES: CC1(C)c2cc3cc(-c4nc(-c5ccccc5)nc(-c5ccccc5)n4)ccc3cc2-c2ccc3c(ccc4nc(-c5ccccc5)oc43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)oc43)c21]

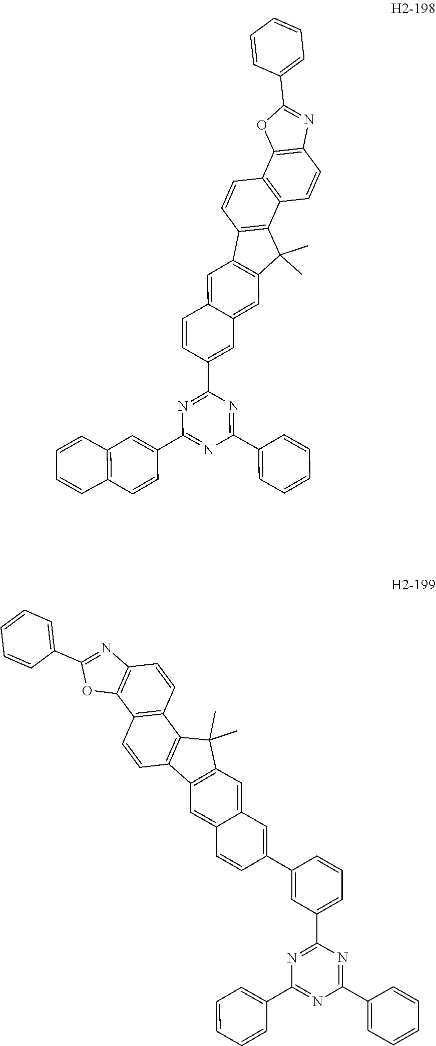 [SMILES: CC1(C)c2cc3cc(-c4nc(-c5ccccc5)nc(-c5ccc6ccccc6c5)n4)ccc3cc2-c2ccc3c(ccc4nc(-c5ccccc5)oc43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)oc43)c21]

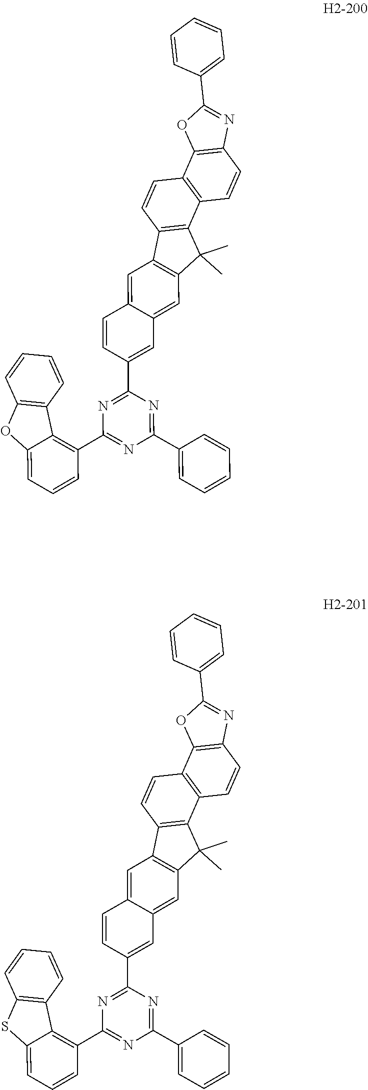 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)oc43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)oc43)c21]

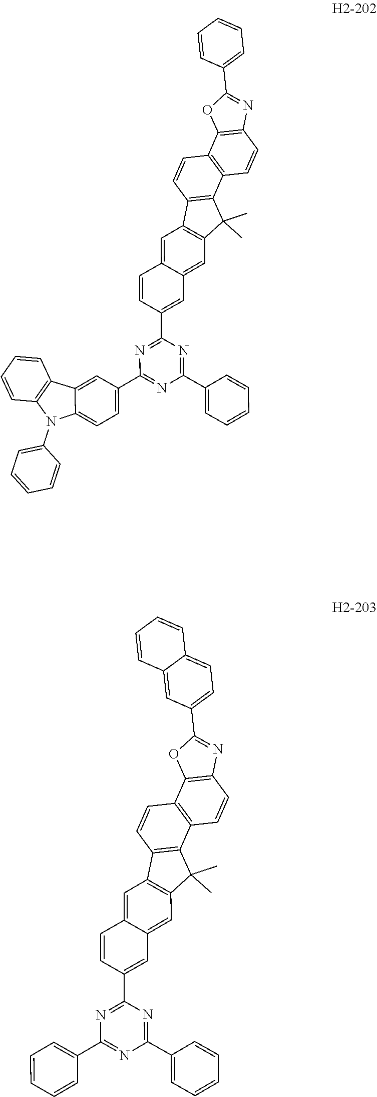 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)oc43)c21 ; CC1(C)c2cc3cc(-c4nc(-c5ccccc5)nc(-c5ccccc5)n4)ccc3cc2-c2ccc3c(ccc4nc(-c5ccc6ccccc6c5)oc43)c21]

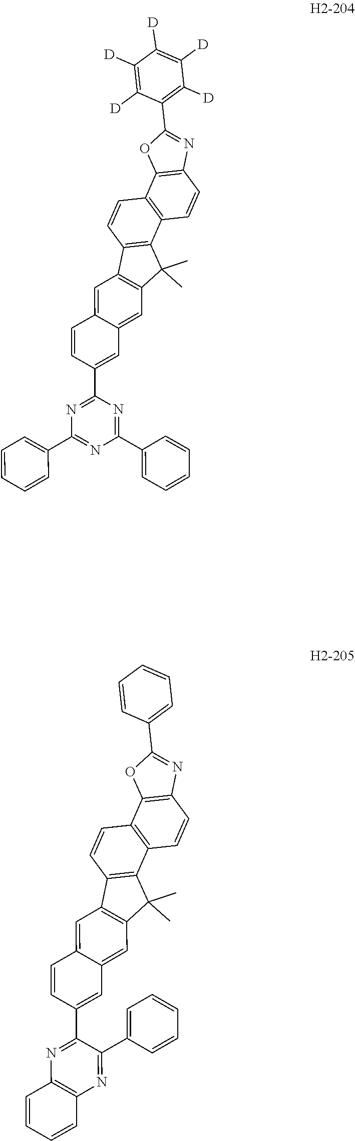 [SMILES: [2H]c1c([2H])c([2H])c(-c2nc3ccc4c5c(ccc4c3o2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)oc43)c21]

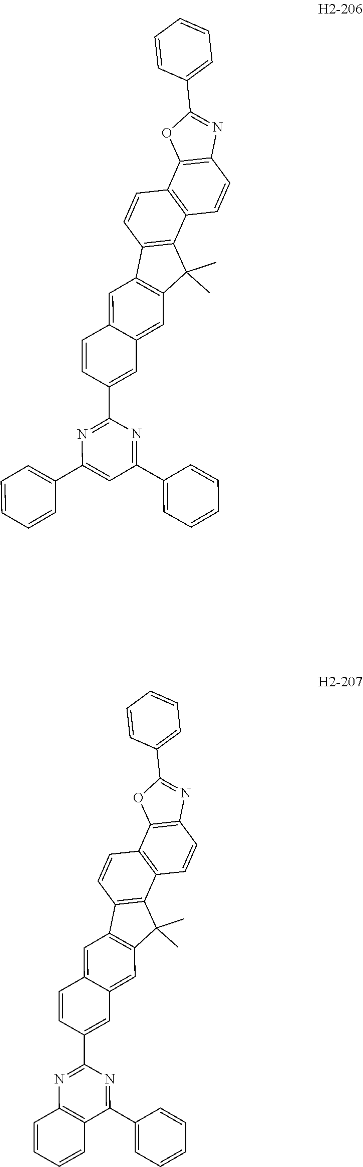 [SMILES: CC1(C)c2cc3cc(-c4nc(-c5ccccc5)cc(-c5ccccc5)n4)ccc3cc2-c2ccc3c(ccc4nc(-c5ccccc5)oc43)c21 ; CC1(C)c2cc3cc(-c4nc(-c5ccccc5)c5ccccc5n4)ccc3cc2-c2ccc3c(ccc4nc(-c5ccccc5)oc43)c21]

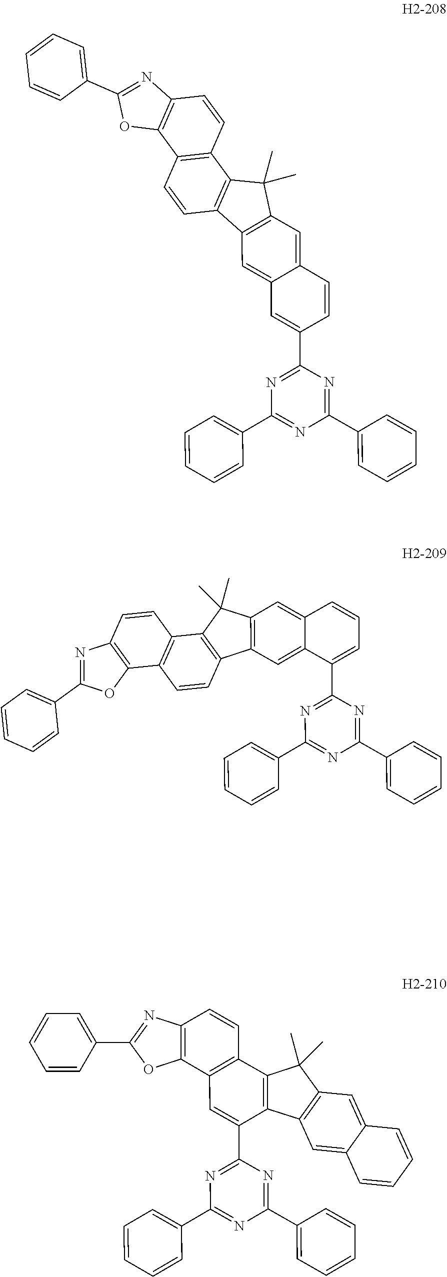 [SMILES: CC1(C)c2cc3ccc(-c4nc(-c5ccccc5)nc(-c5ccccc5)n4)cc3cc2-c2ccc3c(ccc4nc(-c5ccccc5)oc43)c21 ; CC1(C)c2cc3cccc(-c4nc(-c5ccccc5)nc(-c5ccccc5)n4)c3cc2-c2ccc3c(ccc4nc(-c5ccccc5)oc43)c21 ; CC1(C)c2cc3ccccc3cc2-c2c(-c3nc(-c4ccccc4)nc(-c4ccccc4)n3)cc3c(ccc4nc(-c5ccccc5)oc43)c21]

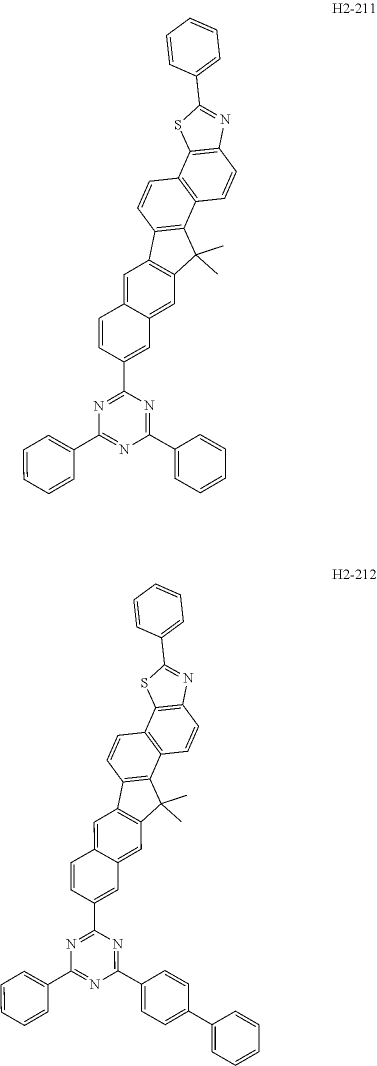 [SMILES: CC1(C)c2cc3cc(-c4nc(-c5ccccc5)nc(-c5ccccc5)n4)ccc3cc2-c2ccc3c(ccc4nc(-c5ccccc5)sc43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)sc43)c21]

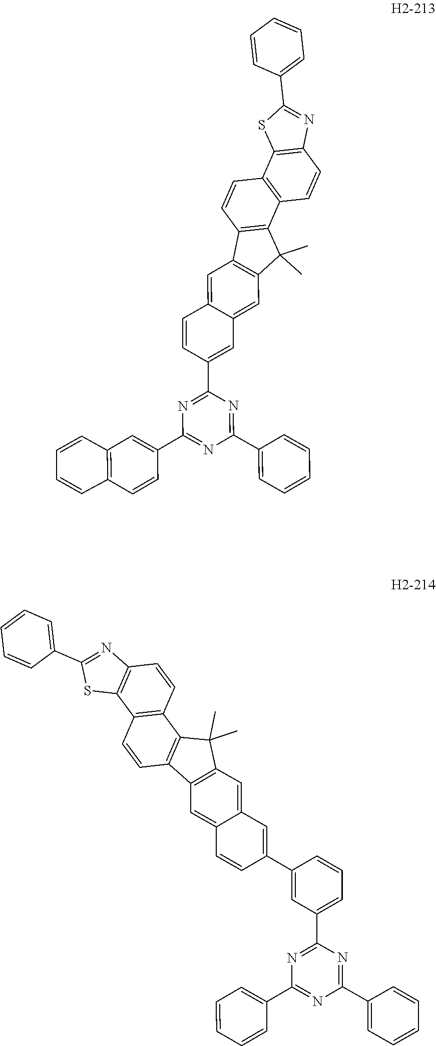 [SMILES: CC1(C)c2cc3cc(-c4nc(-c5ccccc5)nc(-c5ccc6ccccc6c5)n4)ccc3cc2-c2ccc3c(ccc4nc(-c5ccccc5)sc43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)sc43)c21]

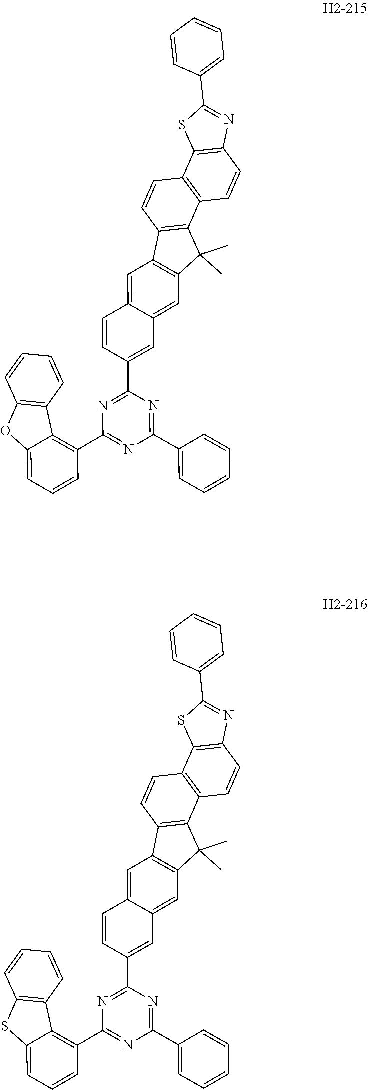 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)sc43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)sc43)c21]

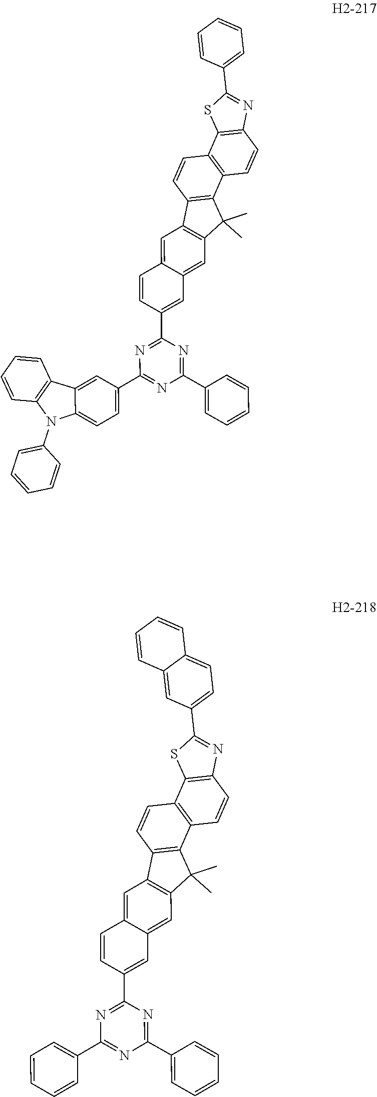 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)sc43)c21 ; CC1(C)c2cc3cc(-c4nc(-c5ccccc5)nc(-c5ccccc5)n4)ccc3cc2-c2ccc3c(ccc4nc(-c5ccc6ccccc6c5)sc43)c21]

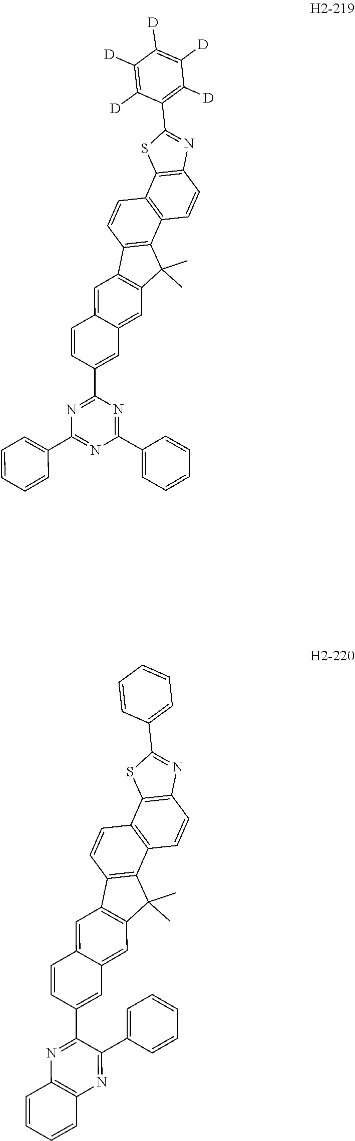 [SMILES: [2H]c1c([2H])c([2H])c(-c2nc3ccc4c5c(ccc4c3s2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)sc43)c21]

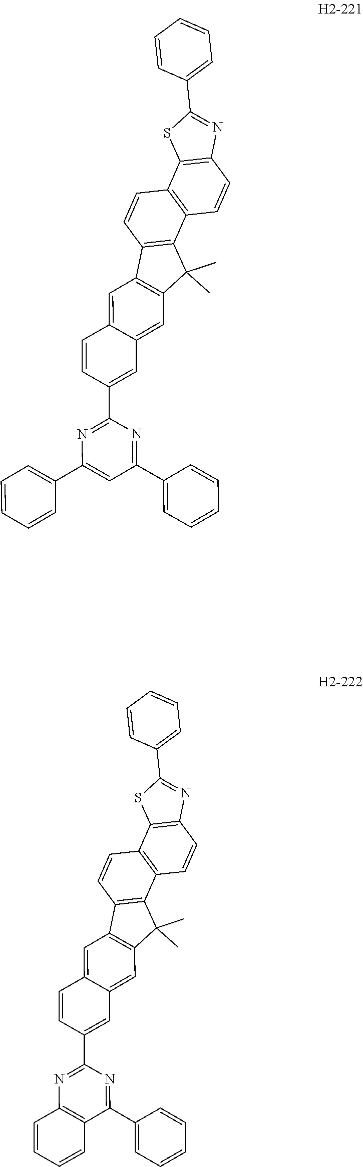 [SMILES: CC1(C)c2cc3cc(-c4nc(-c5ccccc5)cc(-c5ccccc5)n4)ccc3cc2-c2ccc3c(ccc4nc(-c5ccccc5)sc43)c21 ; CC1(C)c2cc3cc(-c4nc(-c5ccccc5)c5ccccc5n4)ccc3cc2-c2ccc3c(ccc4nc(-c5ccccc5)sc43)c21]

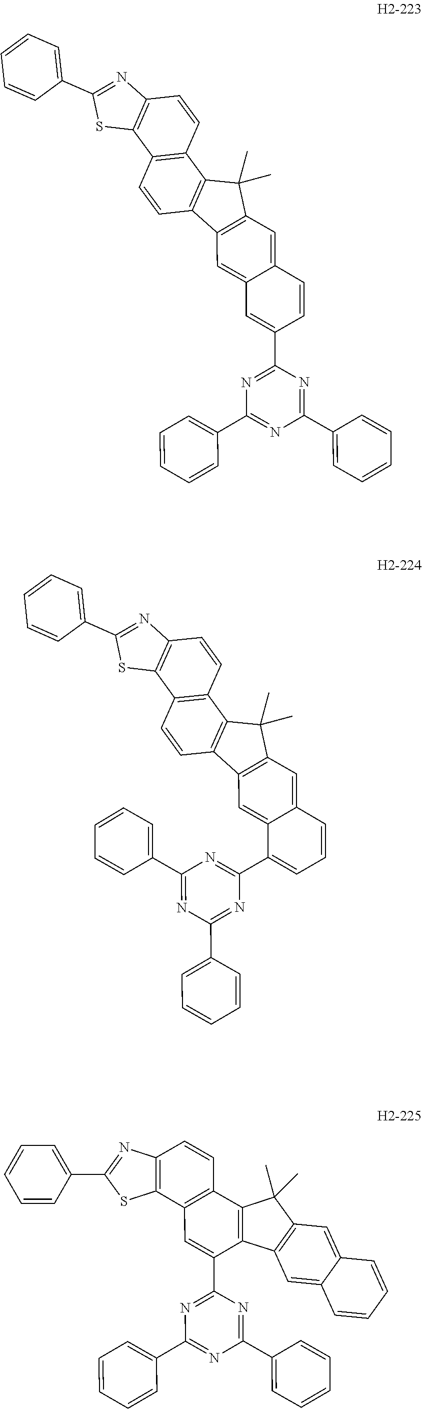 [SMILES: CC1(C)c2cc3ccc(-c4nc(-c5ccccc5)nc(-c5ccccc5)n4)cc3cc2-c2ccc3c(ccc4nc(-c5ccccc5)sc43)c21 ; CC1(C)c2cc3cccc(-c4nc(-c5ccccc5)nc(-c5ccccc5)n4)c3cc2-c2ccc3c(ccc4nc(-c5ccccc5)sc43)c21 ; CC1(C)c2cc3ccccc3cc2-c2c(-c3nc(-c4ccccc4)nc(-c4ccccc4)n3)cc3c(ccc4nc(-c5ccccc5)sc43)c21]

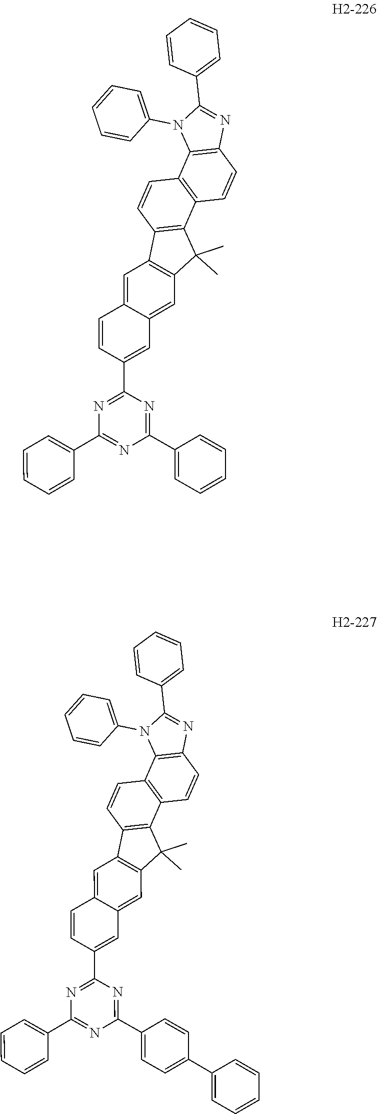 [SMILES: CC1(C)c2cc3cc(-c4nc(-c5ccccc5)nc(-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)n(-c5ccccc5)c43)c21]

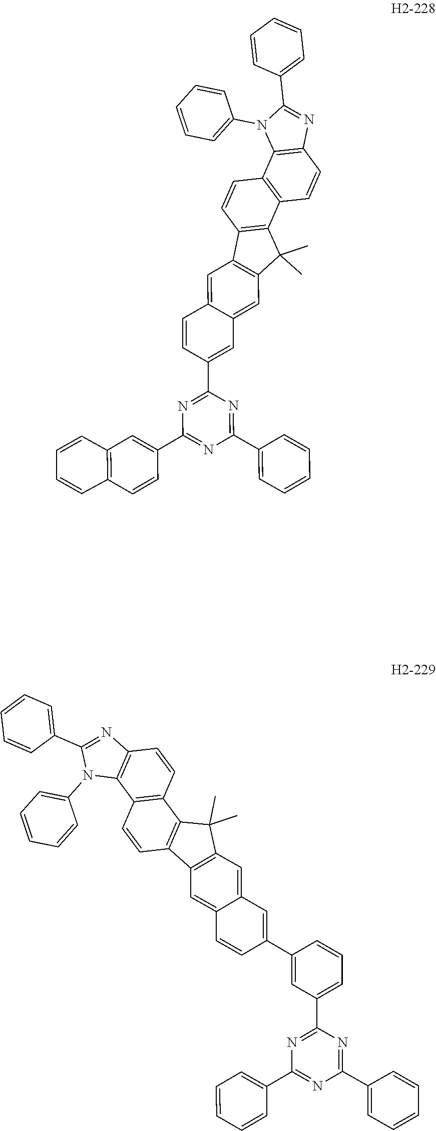 [SMILES: CC1(C)c2cc3cc(-c4nc(-c5ccccc5)nc(-c5ccc6ccccc6c5)n4)ccc3cc2-c2ccc3c(ccc4nc(-c5ccccc5)n(-c5ccccc5)c43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)n(-c5ccccc5)c43)c21]

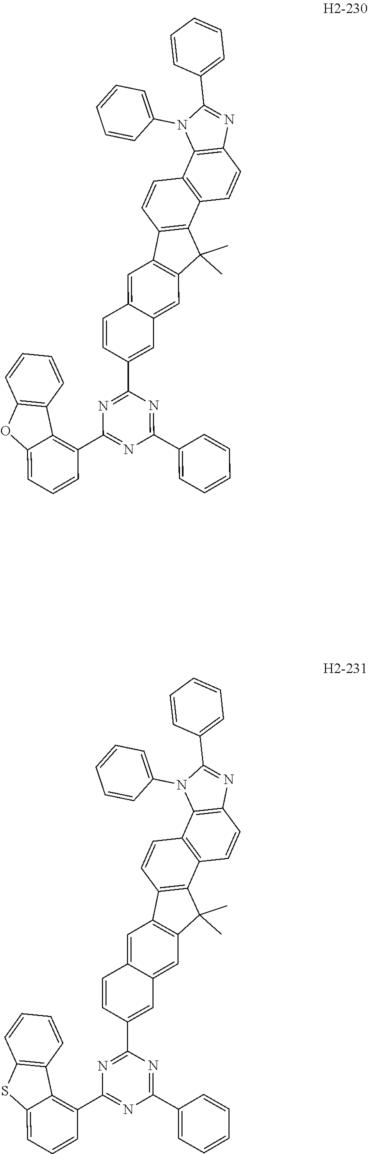 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)n(-c5ccccc5)c43)c21]

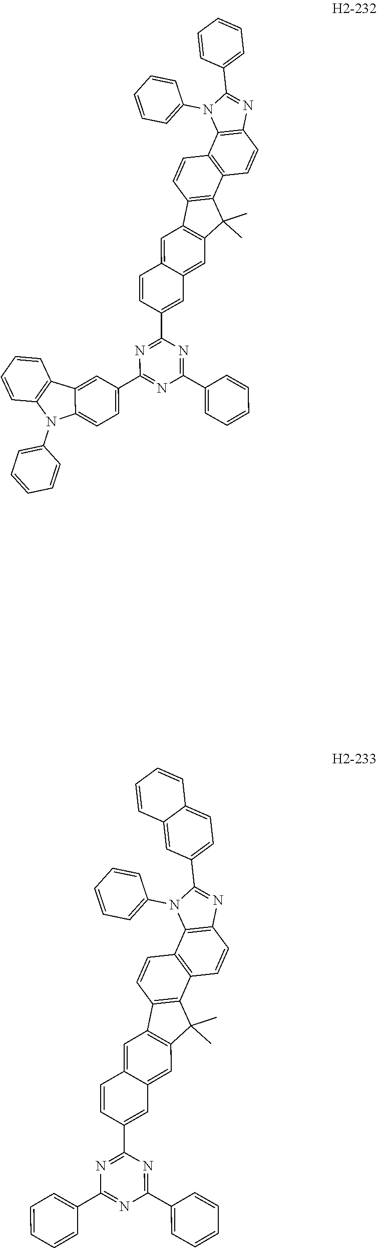 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccccc5)n4)ccc3cc2-c2ccc3c(ccc4nc(-c5ccc6ccccc6c5)n(-c5ccccc5)c43)c21]

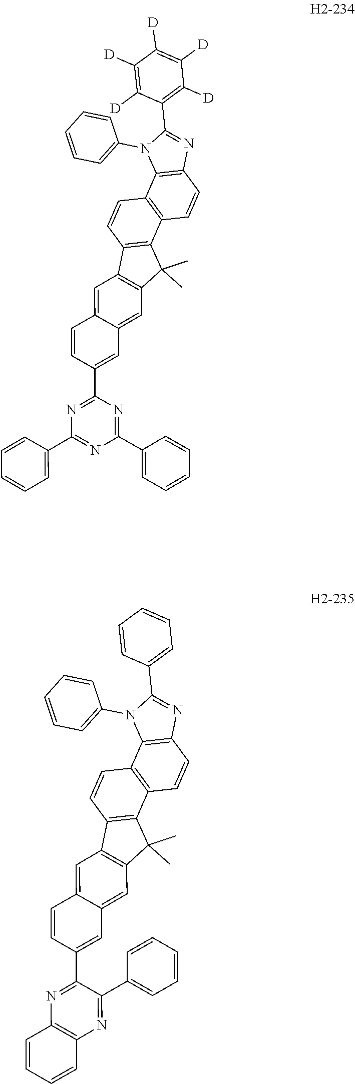 [SMILES: [2H]c1c([2H])c([2H])c(-c2nc3ccc4c5c(ccc4c3n2-c2ccccc2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)n(-c5ccccc5)c43)c21]

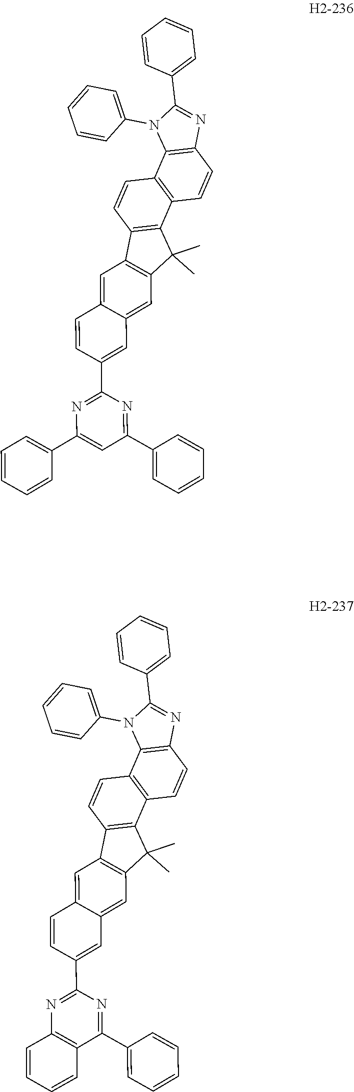 [SMILES: CC1(C)c2cc3cc(-c4nc(-c5ccccc5)cc(-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)c5ccccc5n4)ccc3cc2-c2ccc3c(ccc4nc(-c5ccccc5)n(-c5ccccc5)c43)c21]

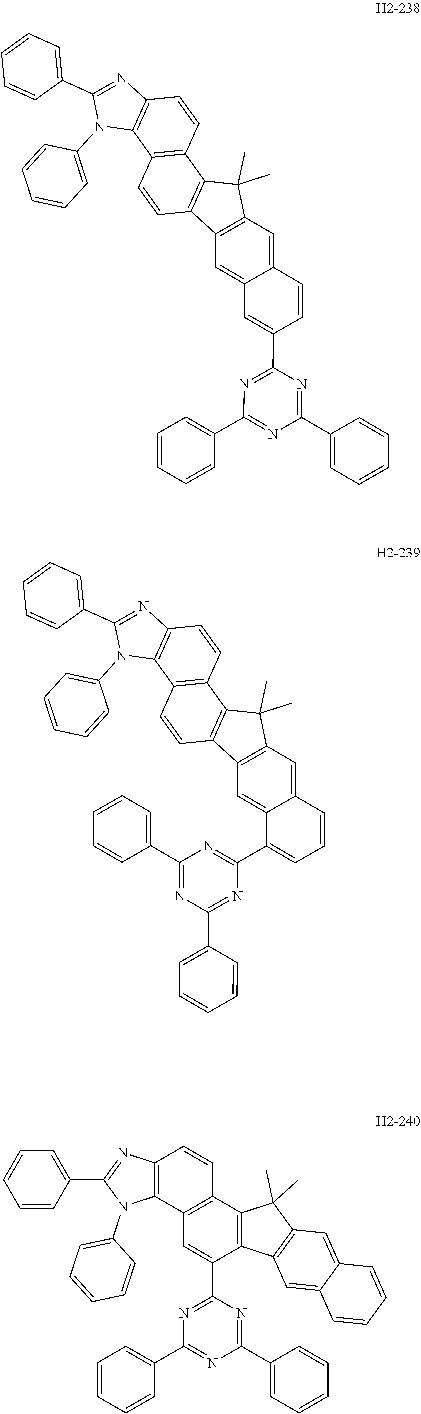 [SMILES: CC1(C)c2cc3ccc(-c4nc(-c5ccccc5)nc(-c5ccccc5)n4)cc3cc2-c2ccc3c(ccc4nc(-c5ccccc5)n(-c5ccccc5)c43)c21 ; CC1(C)c2cc3cccc(-c4nc(-c5ccccc5)nc(-c5ccccc5)n4)c3cc2-c2ccc3c(ccc4nc(-c5ccccc5)n(-c5ccccc5)c43)c21 ; CC1(C)c2cc3ccccc3cc2-c2c(-c3nc(-c4ccccc4)nc(-c4ccccc4)n3)cc3c(ccc4nc(-c5ccccc5)n(-c5ccccc5)c43)c21]

The compound represented by Formula 2 according to the present disclosure can be prepared by referring to the following Reaction Scheme 1, for example, but is not limited thereto.

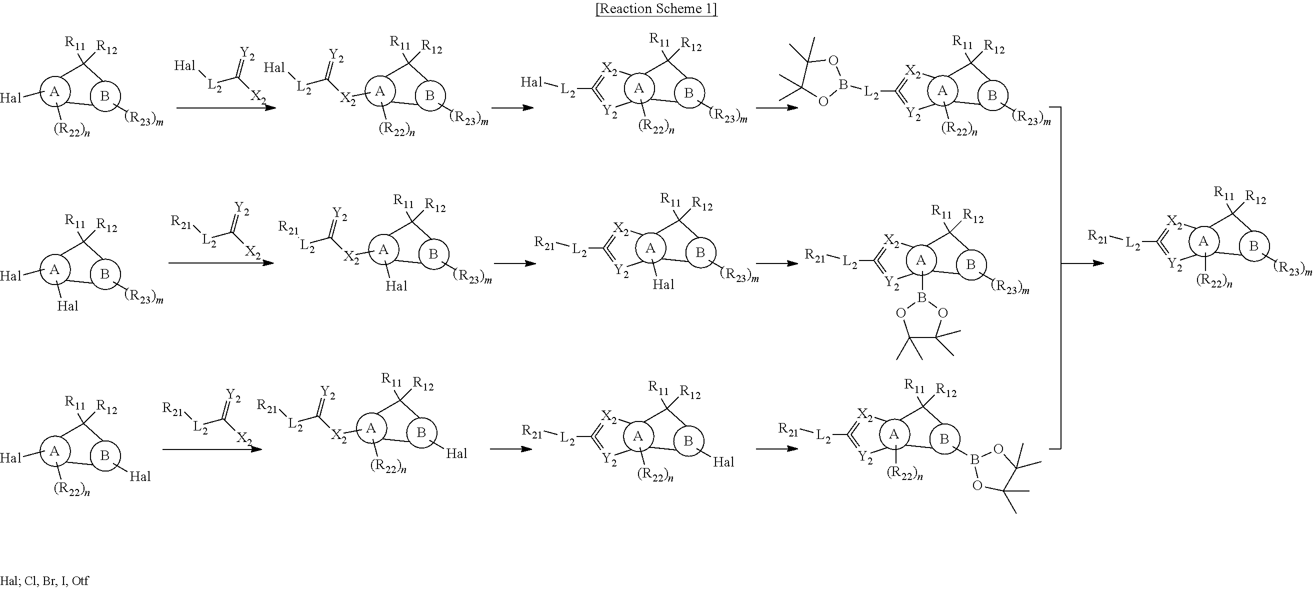

In Reaction Scheme 1, the definition of each of the substituents is as defined in Formula 2, and Hal means halogen.

As described above, exemplary synthesis examples of the compounds represented by Formula 2 according to the present disclosure are described, but they are based on a Buchwald-Hartwig cross coupling reaction, N-arylation reaction, H-mont-mediated etherification reaction, Miyaura borylation reaction, Suzuki cross-coupling reaction, intramolecular acid-induced cyclization reaction, Pd(II)-catalyzed oxidative cyclization reaction, Grignard reaction, Heck reaction, cyclic dehydration reaction, S_{N}1 substitution reaction, S_{N}2 substitution reaction, phosphine-mediated reductive cyclization reaction, etc. It will be understood by one skilled in the art that the above reaction proceeds even if other substituents defined in Formula 2 other than the substituents described in the specific synthesis examples are bonded.

According to another embodiment, the present disclosure provides an organic electroluminescent compound represented by the following Formula 2′.

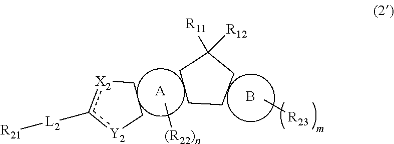

In Formula 2′,
ring A and ring B each independently represent a substituted or unsubstituted (C₆-C₃₀)arene, or a substituted or unsubstituted (3- to 30-membered)heteroarene;
R₁₁ and R₁₂ each independently represent a substituted or unsubstituted (C₁-C₃₀)alkyl, a substituted or unsubstituted (C₆-C₃₀)aryl, or a substituted or unsubstituted (3-to 30-membered)heteroaryl; or may be linked to each other to form a ring(s);
X₂ and Y₂ each independently represent —N=, —NR₂₄, —O—, or —S—; provided that any one of X₂ and Y₂ is —N=, and the other of X₂ and Y₂ is —NR₂₄—, —O—, or —S—;
R₂₁ and R₂₄ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and a (C₆-C₃₀) aromatic ring, or —L₃-HAr;
provided that at least one of R₂₁ to R₂₄ is —L₃-HAr, and if R₂₃ is —L₃-HAr, the case where ring A and ring B are both a substituted or unsubstituted benzene is excluded;
L₂ and L₃ each independently represent a single bond, a substituted or unsubstituted (C₆-C₃₀)arylene, or a substituted or unsubstituted (3- to 30-membered)heteroarylene;
HAr represents a substituted or unsubstituted (3- to 30-membered)heteroaryl containing at least one nitrogen atom; and
n represents an integer of 1 to 4, m represents an integer of 1 to 8, and when n and m are an integer of 2 or more, each of R₂₂ and R₂₃ may be the same or different.

According to one embodiment, the organic electroluminescent compound represented by Formula 2′ may be represented by any one of the following Formulas 2-1a and 2-2 to 2-8.

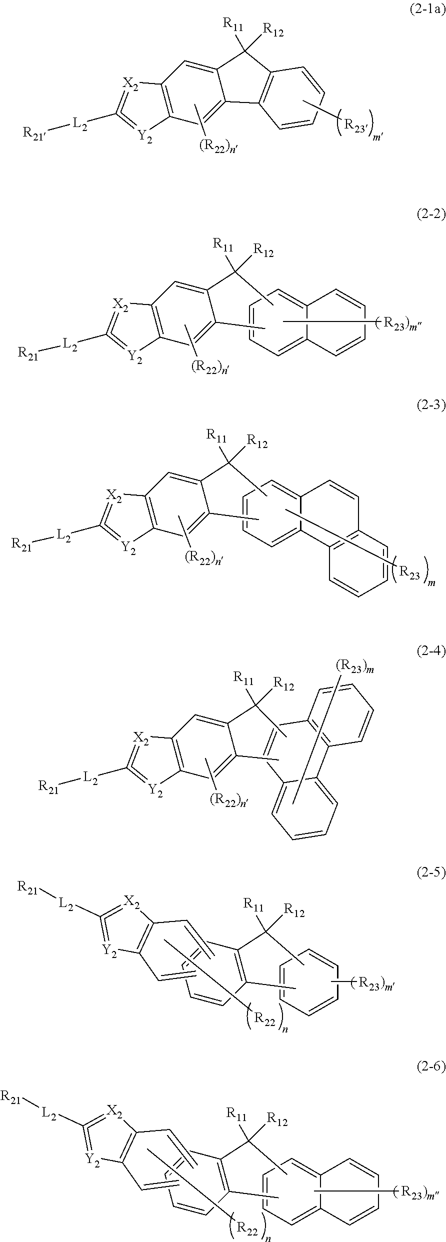

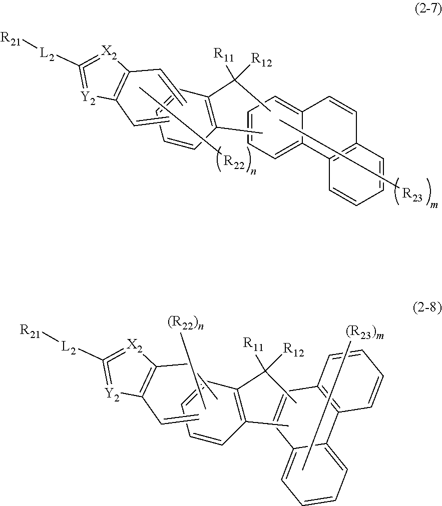

In Formulas 2-1a and 2-2 to 2-8,
R_{21′} and R_{22′} each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and a (C₆-C₃₀) aromatic ring, or —L₃-HAr; provided that any one of R₂₁ and R₂₂— is —L₃-HAr;
R_{23′} represents hydrogen, deuterium, or (C₆-C₃₀)aryl unsubstituted or substituted with deuterium;
n′ represents an integer of 1 or 2, m′ represents an integer of 1 to 4, and m″ represents an integer of 1 to 6; and
X₂, Y₂, R₁₁, R₁₂, R₂₁ to R₂₃, L₂, n, and m are as defined in Formula 2′.

According to another embodiment, the organic electroluminescent compound represented by Formula 2′ may be represented by the following Formula 2-9.

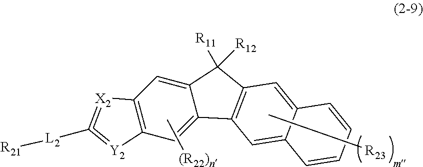

In Formula 2-9,
X₂, Y₂, R₁₁, R₁₂, R₂₁ to R₂₃, and L₂ are as defined in Formula 2′; and
n′ represents an integer of 1 or 2, and m″ represents an integer of 1 to 6.

According to one embodiment, the organic electroluminescent compound represented by Formula 2′ above may be more specifically illustrated by the following compounds, but is not limited thereto.

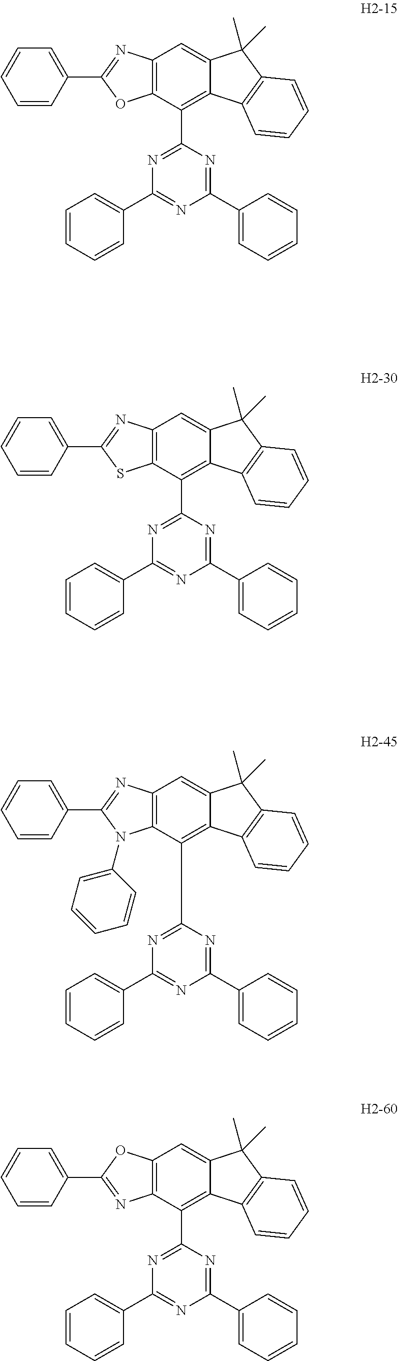 [SMILES: CC1(C)c2ccccc2-c2c1cc1nc(-c3ccccc3)oc1c2-c1nc(-c2ccccc2)nc(-c2ccccc2)n1 ; CC1(C)c2ccccc2-c2c1cc1nc(-c3ccccc3)sc1c2-c1nc(-c2ccccc2)nc(-c2ccccc2)n1 ; CC1(C)c2ccccc2-c2c1cc1nc(-c3ccccc3)n(-c3ccccc3)c1c2-c1nc(-c2ccccc2)nc(-c2ccccc2)n1 ; CC1(C)c2ccccc2-c2c1cc1oc(-c3ccccc3)nc1c2-c1nc(-c2ccccc2)nc(-c2ccccc2)n1]

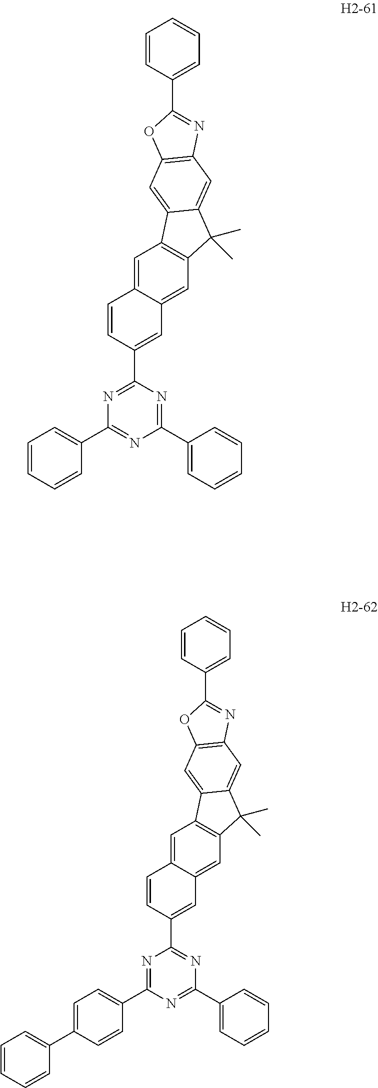 [SMILES: CC1(C)c2cc3cc(-c4nc(-c5ccccc5)nc(-c5ccccc5)n4)ccc3cc2-c2cc3oc(-c4ccccc4)nc3cc21 ; CC1(C)c2cc3cc(-c4nc(-c5ccccc5)nc(-c5ccc(-c6ccccc6)cc5)n4)ccc3cc2-c2cc3oc(-c4ccccc4)nc3cc21]

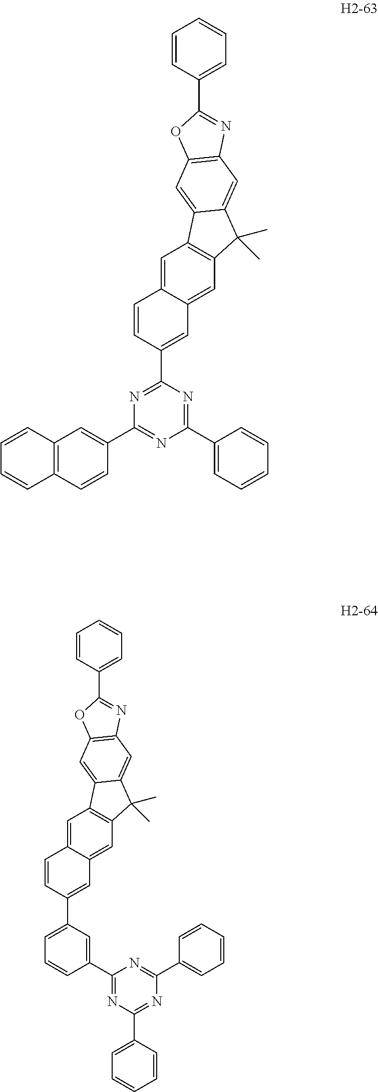 [SMILES: CC1(C)c2cc3cc(-c4nc(-c5ccccc5)nc(-c5ccc6ccccc6c5)n4)ccc3cc2-c2cc3oc(-c4ccccc4)nc3cc21 ; CC1(C)c2cc3cc(-c4cccc(-c5nc(-c6ccccc6)nc(-c6ccccc6)n5)c4)ccc3cc2-c2cc3oc(-c4ccccc4)nc3cc21]

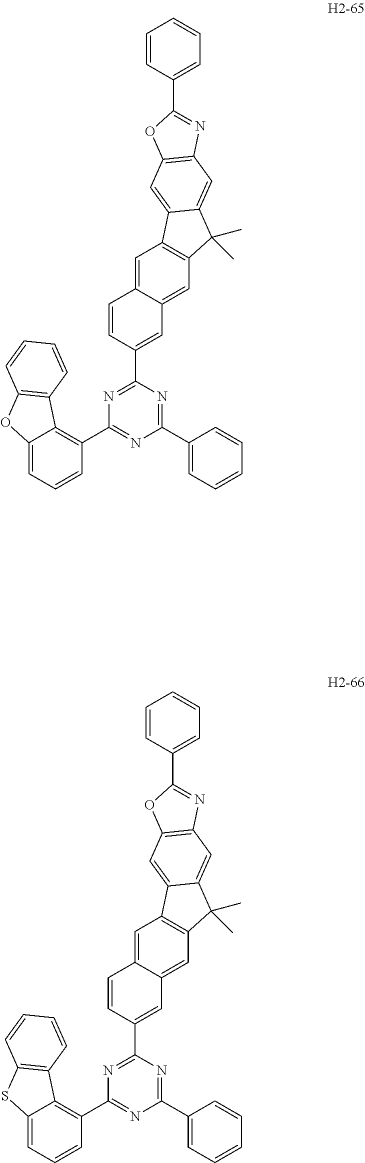 [SMILES: CC1(C)c2cc3cc(-c4nc(-c5ccccc5)nc(-c5cccc6oc7ccccc7c56)n4)ccc3cc2-c2cc3oc(-c4ccccc4)nc3cc21 ; CC1(C)c2cc3cc(-c4nc(-c5ccccc5)nc(-c5cccc6sc7ccccc7c56)n4)ccc3cc2-c2cc3oc(-c4ccccc4)nc3cc21]

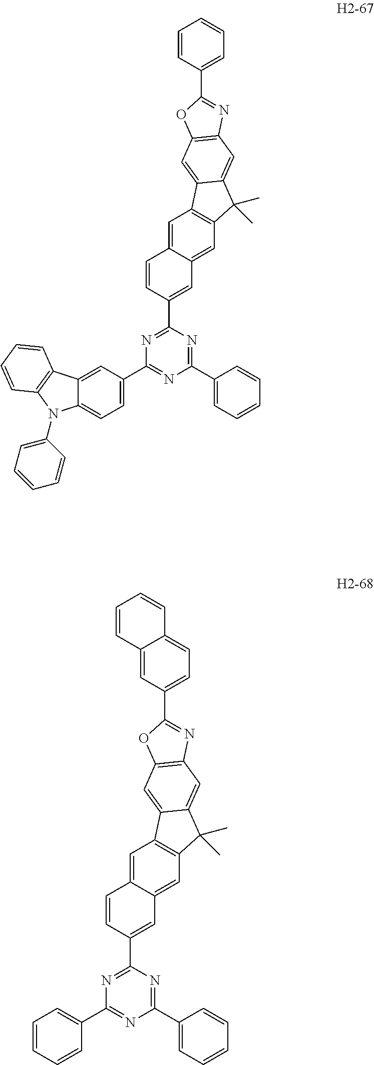 [SMILES: CC1(C)c2cc3cc(-c4nc(-c5ccccc5)nc(-c5ccc6c(c5)c5ccccc5n6-c5ccccc5)n4)ccc3cc2-c2cc3oc(-c4ccccc4)nc3cc21 ; CC1(C)c2cc3cc(-c4nc(-c5ccccc5)nc(-c5ccccc5)n4)ccc3cc2-c2cc3oc(-c4ccc5ccccc5c4)nc3cc21]

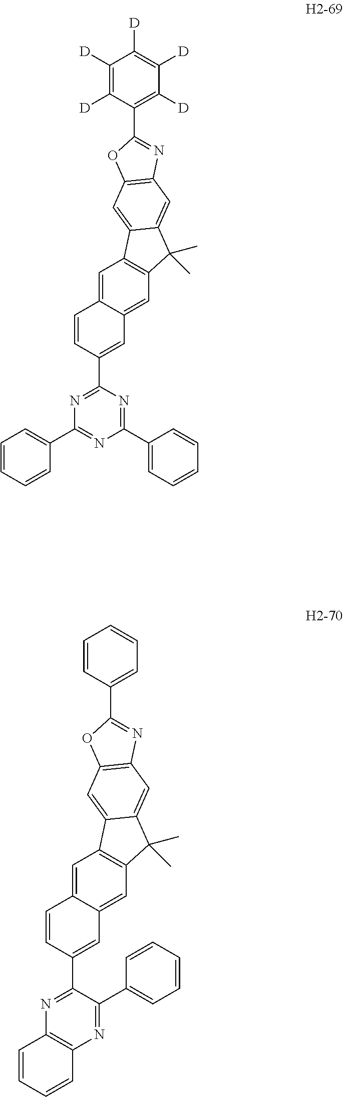 [SMILES: [2H]c1c([2H])c([2H])c(-c2nc3cc4c(cc3o2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3oc(-c4ccccc4)nc3cc21]

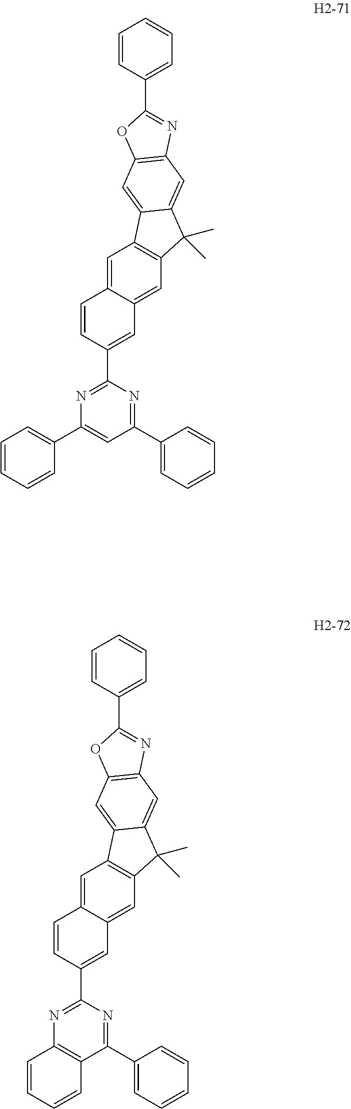 [SMILES: CC1(C)c2cc3cc(-c4nc(-c5ccccc5)cc(-c5ccccc5)n4)ccc3cc2-c2cc3oc(-c4ccccc4)nc3cc21 ; CC1(C)c2cc3cc(-c4nc(-c5ccccc5)c5ccccc5n4)ccc3cc2-c2cc3oc(-c4ccccc4)nc3cc21]

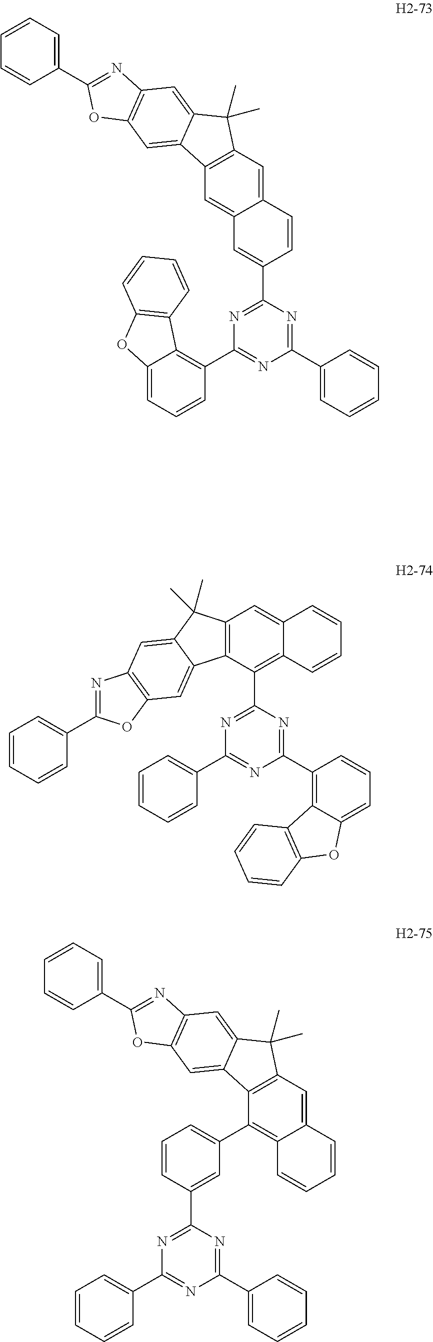 [SMILES: CC1(C)c2cc3ccc(-c4nc(-c5ccccc5)nc(-c5cccc6oc7ccccc7c56)n4)cc3cc2-c2cc3oc(-c4ccccc4)nc3cc21 ; CC1(C)c2cc3nc(-c4ccccc4)oc3cc2-c2c1cc1ccccc1c2-c1nc(-c2ccccc2)nc(-c2cccc3oc4ccccc4c23)n1 ; CC1(C)c2cc3nc(-c4ccccc4)oc3cc2-c2c1cc1ccccc1c2-c1cccc(-c2nc(-c3ccccc3)nc(-c3ccccc3)n2)c1]

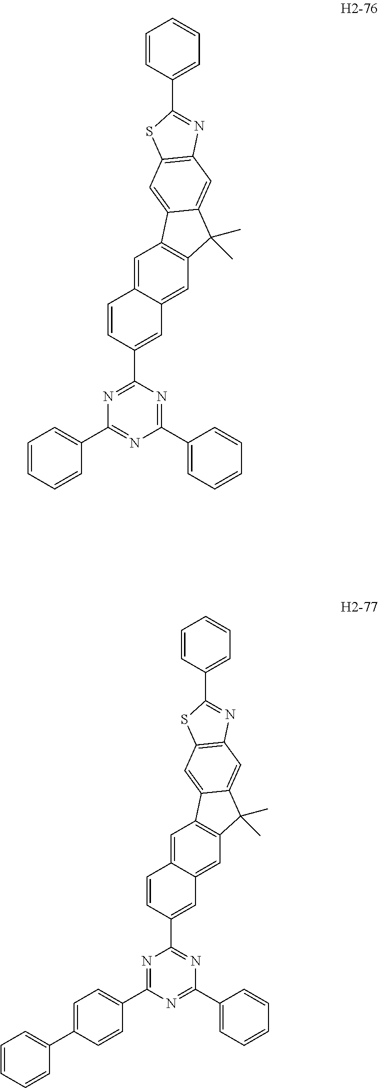 [SMILES: CC1(C)c2cc3cc(-c4nc(-c5ccccc5)nc(-c5ccccc5)n4)ccc3cc2-c2cc3sc(-c4ccccc4)nc3cc21 ; CC1(C)c2cc3cc(-c4nc(-c5ccccc5)nc(-c5ccc(-c6ccccc6)cc5)n4)ccc3cc2-c2cc3sc(-c4ccccc4)nc3cc21]

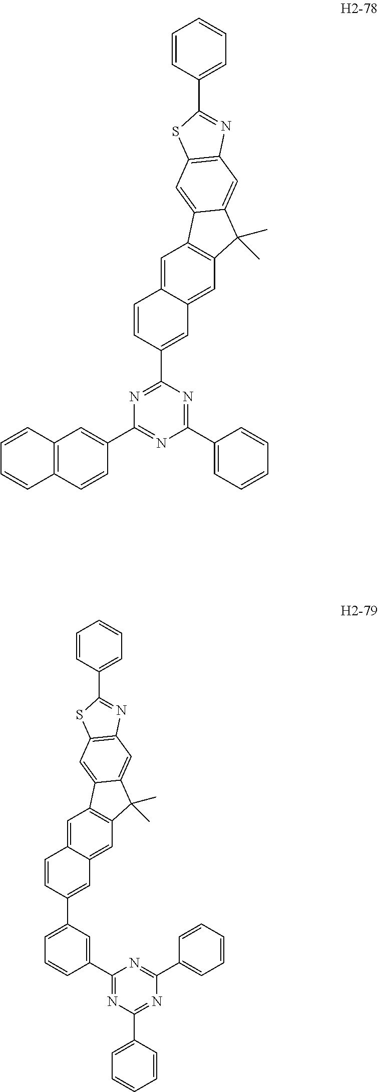 [SMILES: CC1(C)c2cc3cc(-c4nc(-c5ccccc5)nc(-c5ccc6ccccc6c5)n4)ccc3cc2-c2cc3sc(-c4ccccc4)nc3cc21 ; CC1(C)c2cc3cc(-c4cccc(-c5nc(-c6ccccc6)nc(-c6ccccc6)n5)c4)ccc3cc2-c2cc3sc(-c4ccccc4)nc3cc21]

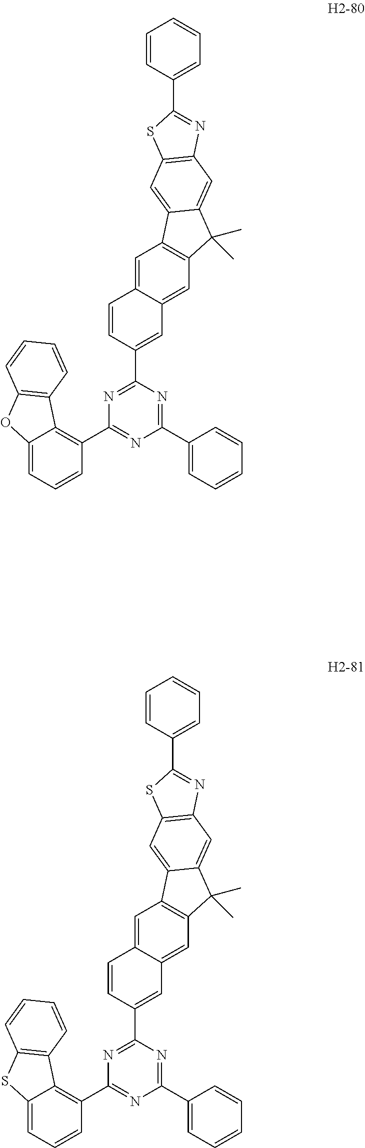 [SMILES: CC1(C)c2cc3cc(-c4nc(-c5ccccc5)nc(-c5cccc6oc7ccccc7c56)n4)ccc3cc2-c2cc3sc(-c4ccccc4)nc3cc21 ; CC1(C)c2cc3cc(-c4nc(-c5ccccc5)nc(-c5cccc6sc7ccccc7c56)n4)ccc3cc2-c2cc3sc(-c4ccccc4)nc3cc21]

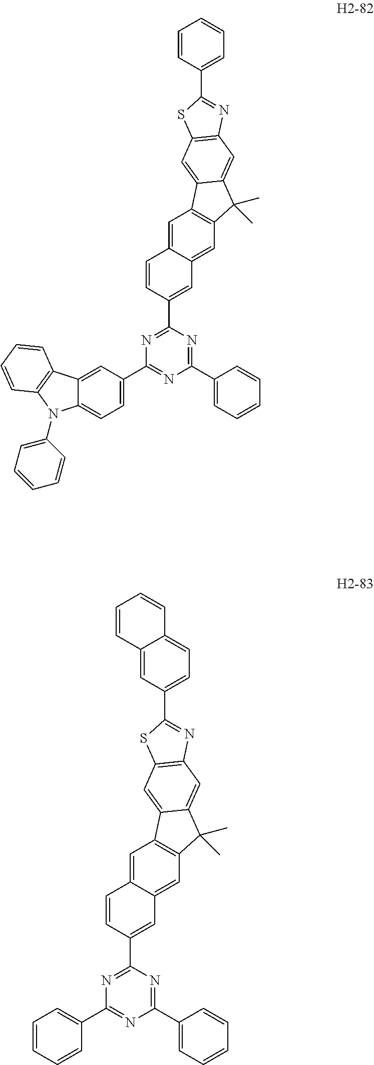 [SMILES: CC1(C)c2cc3cc(-c4nc(-c5ccccc5)nc(-c5ccc6c(c5)c5ccccc5n6-c5ccccc5)n4)ccc3cc2-c2cc3sc(-c4ccccc4)nc3cc21 ; CC1(C)c2cc3cc(-c4nc(-c5ccccc5)nc(-c5ccccc5)n4)ccc3cc2-c2cc3sc(-c4ccc5ccccc5c4)nc3cc21]

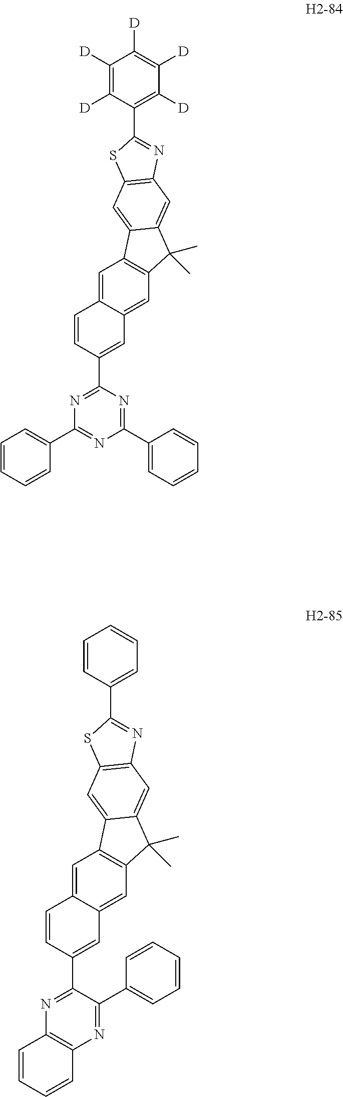 [SMILES: [2H]c1c([2H])c([2H])c(-c2nc3cc4c(cc3s2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3sc(-c4ccccc4)nc3cc21]

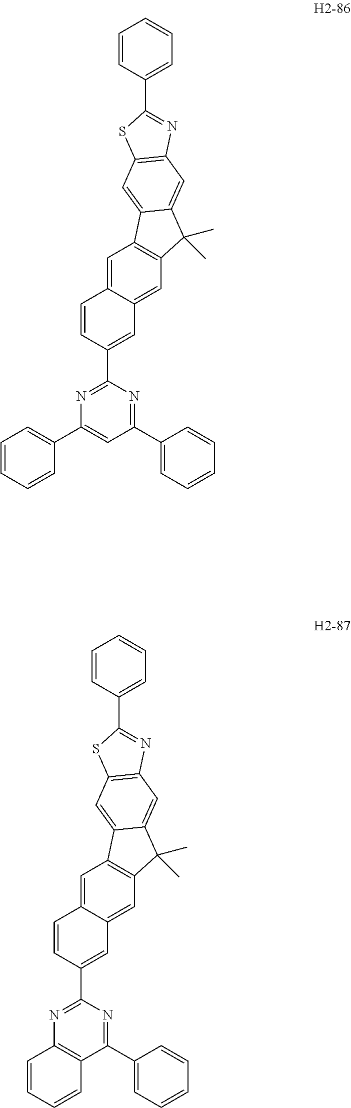 [SMILES: CC1(C)c2cc3cc(-c4nc(-c5ccccc5)cc(-c5ccccc5)n4)ccc3cc2-c2cc3sc(-c4ccccc4)nc3cc21 ; CC1(C)c2cc3cc(-c4nc(-c5ccccc5)c5ccccc5n4)ccc3cc2-c2cc3sc(-c4ccccc4)nc3cc21]

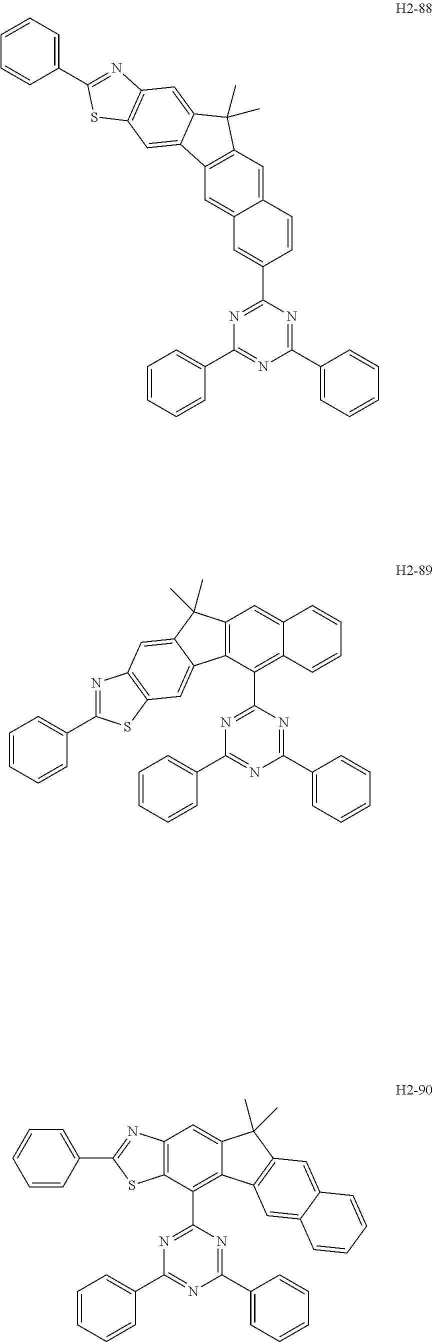 [SMILES: CC1(C)c2cc3ccc(-c4nc(-c5ccccc5)nc(-c5ccccc5)n4)cc3cc2-c2cc3sc(-c4ccccc4)nc3cc21 ; CC1(C)c2cc3nc(-c4ccccc4)sc3cc2-c2c1cc1ccccc1c2-c1nc(-c2ccccc2)nc(-c2ccccc2)n1 ; CC1(C)c2cc3ccccc3cc2-c2c1cc1nc(-c3ccccc3)sc1c2-c1nc(-c2ccccc2)nc(-c2ccccc2)n1]

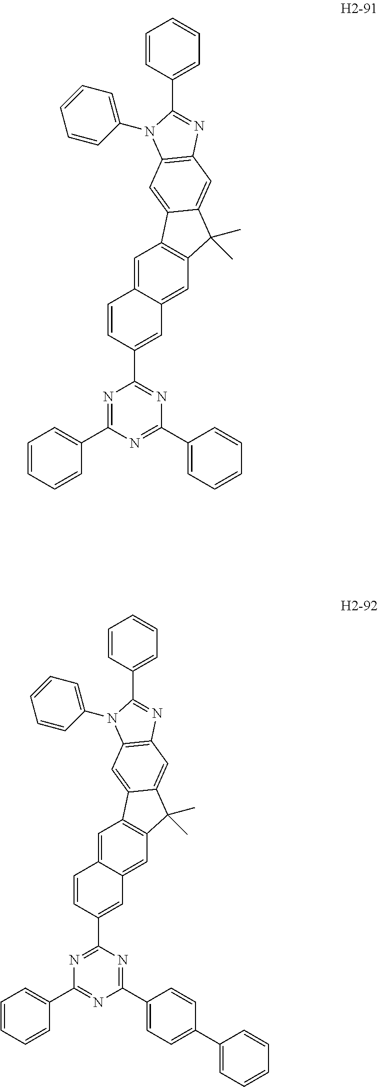 [SMILES: CC1(C)c2cc3cc(-c4nc(-c5ccccc5)nc(-c5ccccc5)n4)ccc3cc2-c2cc3c(cc21)nc(-c1ccccc1)n3-c1ccccc1 ; CC1(C)c2cc3cc(-c4nc(-c5ccccc5)nc(-c5ccc(-c6ccccc6)cc5)n4)ccc3cc2-c2cc3c(cc21)nc(-c1ccccc1)n3-c1ccccc1]

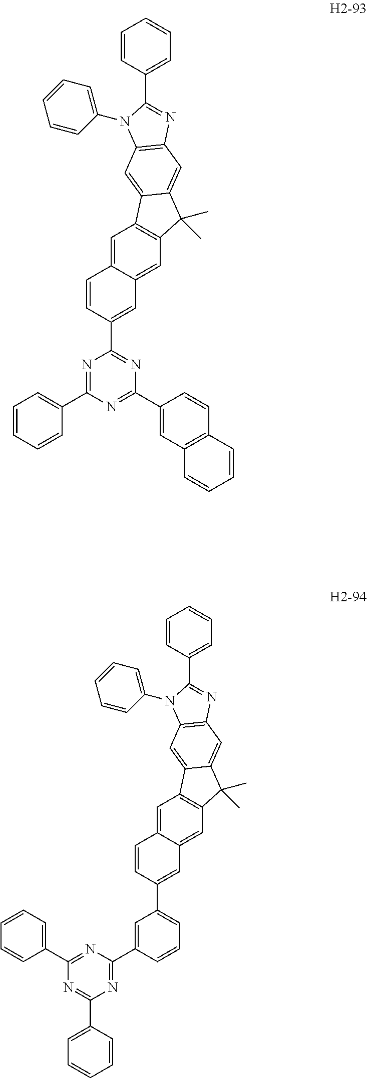 [SMILES: CC1(C)c2cc3cc(-c4nc(-c5ccccc5)nc(-c5ccc6ccccc6c5)n4)ccc3cc2-c2cc3c(cc21)nc(-c1ccccc1)n3-c1ccccc1 ; CC1(C)c2cc3cc(-c4cccc(-c5nc(-c6ccccc6)nc(-c6ccccc6)n5)c4)ccc3cc2-c2cc3c(cc21)nc(-c1ccccc1)n3-c1ccccc1]

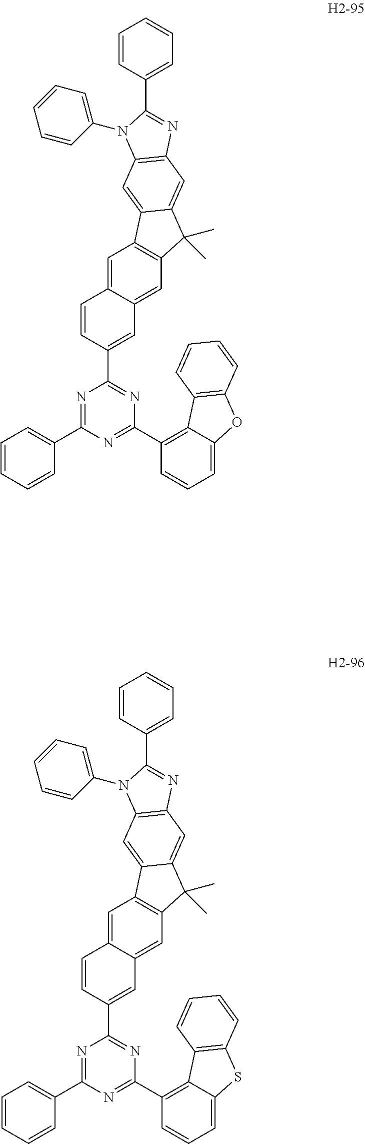 [SMILES: CC1(C)c2cc3cc(-c4nc(-c5ccccc5)nc(-c5cccc6oc7ccccc7c56)n4)ccc3cc2-c2cc3c(cc21)nc(-c1ccccc1)n3-c1ccccc1 ; CC1(C)c2cc3cc(-c4nc(-c5ccccc5)nc(-c5cccc6sc7ccccc7c56)n4)ccc3cc2-c2cc3c(cc21)nc(-c1ccccc1)n3-c1ccccc1]

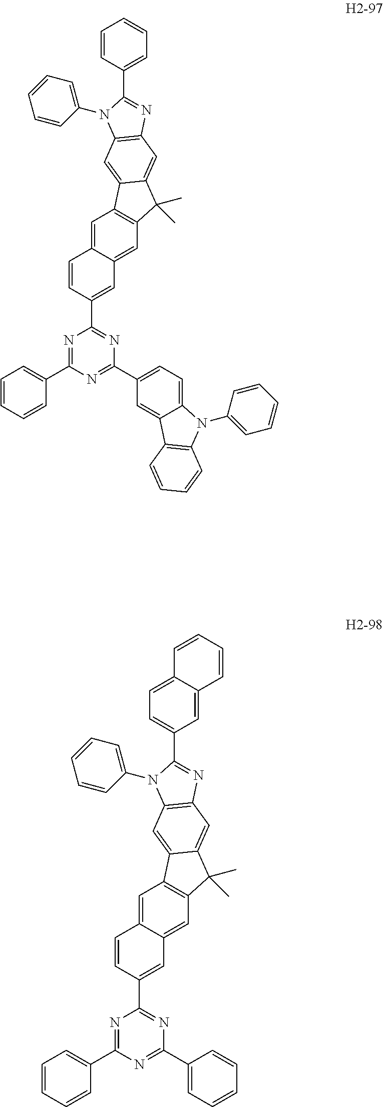 [SMILES: CC1(C)c2cc3cc(-c4nc(-c5ccccc5)nc(-c5ccc6c(c5)c5ccccc5n6-c5ccccc5)n4)ccc3cc2-c2cc3c(cc21)nc(-c1ccccc1)n3-c1ccccc1 ; CC1(C)c2cc3cc(-c4nc(-c5ccccc5)nc(-c5ccccc5)n4)ccc3cc2-c2cc3c(cc21)nc(-c1ccc2ccccc2c1)n3-c1ccccc1]

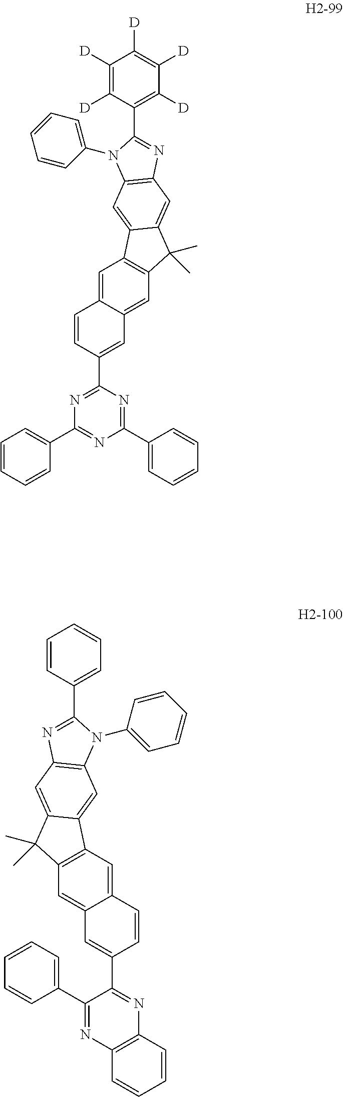 [SMILES: [2H]c1c([2H])c([2H])c(-c2nc3cc4c(cc3n2-c2ccccc2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3c(cc21)nc(-c1ccccc1)n3-c1ccccc1]

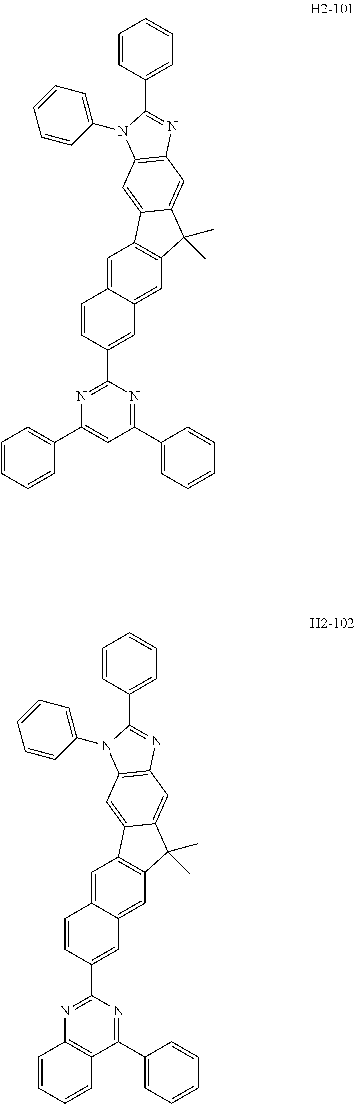 [SMILES: CC1(C)c2cc3cc(-c4nc(-c5ccccc5)cc(-c5ccccc5)n4)ccc3cc2-c2cc3c(cc21)nc(-c1ccccc1)n3-c1ccccc1 ; CC1(C)c2cc3cc(-c4nc(-c5ccccc5)c5ccccc5n4)ccc3cc2-c2cc3c(cc21)nc(-c1ccccc1)n3-c1ccccc1]

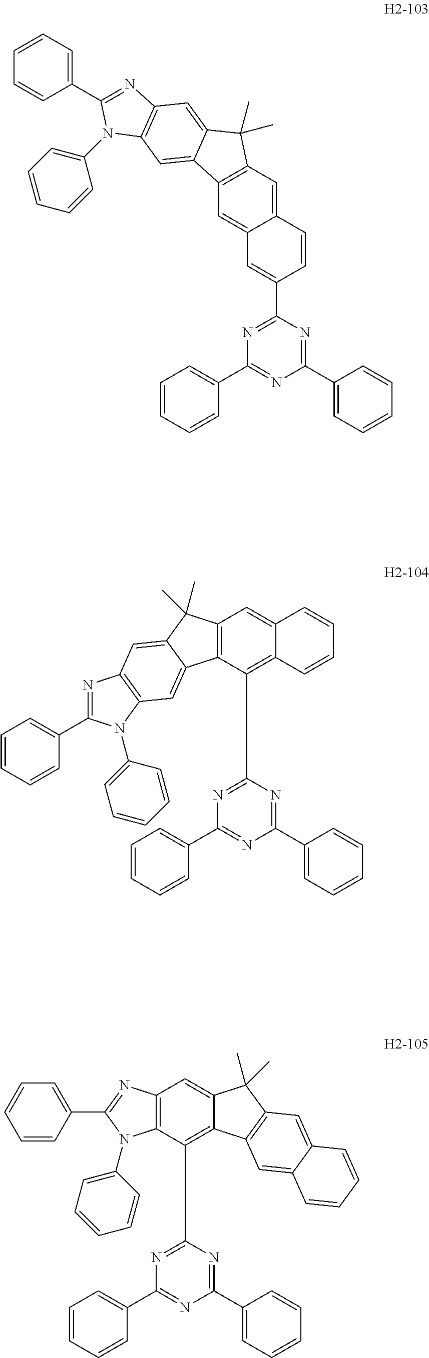 [SMILES: CC1(C)c2cc3ccc(-c4nc(-c5ccccc5)nc(-c5ccccc5)n4)cc3cc2-c2cc3c(cc21)nc(-c1ccccc1)n3-c1ccccc1 ; CC1(C)c2cc3nc(-c4ccccc4)n(-c4ccccc4)c3cc2-c2c1cc1ccccc1c2-c1nc(-c2ccccc2)nc(-c2ccccc2)n1 ; CC1(C)c2cc3ccccc3cc2-c2c1cc1nc(-c3ccccc3)n(-c3ccccc3)c1c2-c1nc(-c2ccccc2)nc(-c2ccccc2)n1]

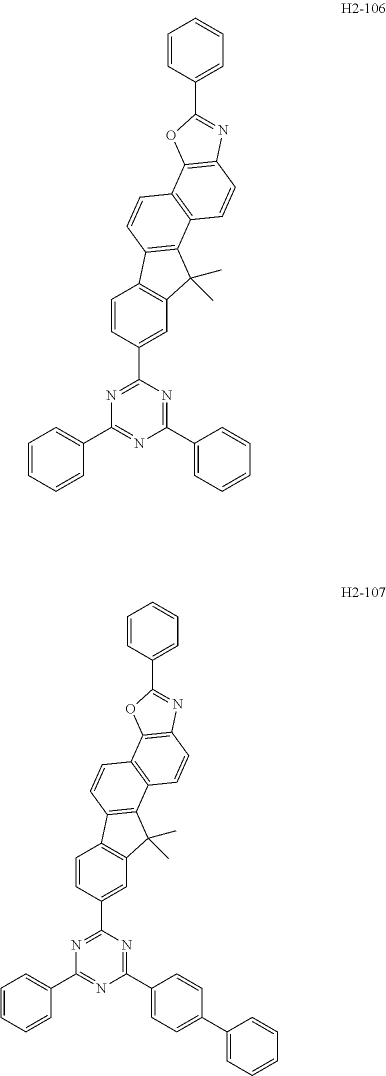 [SMILES: CC1(C)c2cc(-c3nc(-c4ccccc4)nc(-c4ccccc4)n3)ccc2-c2ccc3c(ccc4nc(-c5ccccc5)oc43)c21 ; CC1(C)c2cc(-c3nc(-c4ccccc4)nc(-c4ccc(-c5ccccc5)cc4)n3)ccc2-c2ccc3c(ccc4nc(-c5ccccc5)oc43)c21]

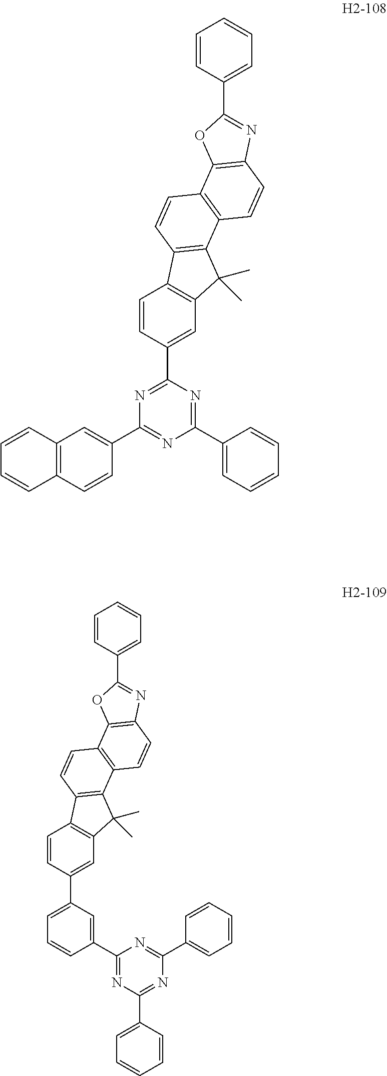 [SMILES: CC1(C)c2cc(-c3nc(-c4ccccc4)nc(-c4ccc5ccccc5c4)n3)ccc2-c2ccc3c(ccc4nc(-c5ccccc5)oc43)c21 ; CC1(C)c2cc(-c3cccc(-c4nc(-c5ccccc5)nc(-c5ccccc5)n4)c3)ccc2-c2ccc3c(ccc4nc(-c5ccccc5)oc43)c21]

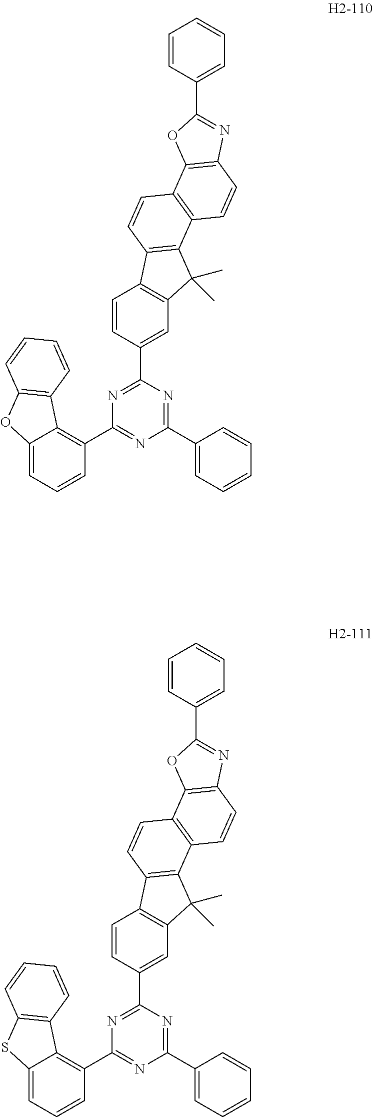 [SMILES: CC1(C)c2cc(-c3nc(-c4ccccc4)nc(-c4cccc5oc6ccccc6c45)n3)ccc2-c2ccc3c(ccc4nc(-c5ccccc5)oc43)c21 ; CC1(C)c2cc(-c3nc(-c4ccccc4)nc(-c4cccc5sc6ccccc6c45)n3)ccc2-c2ccc3c(ccc4nc(-c5ccccc5)oc43)c21]

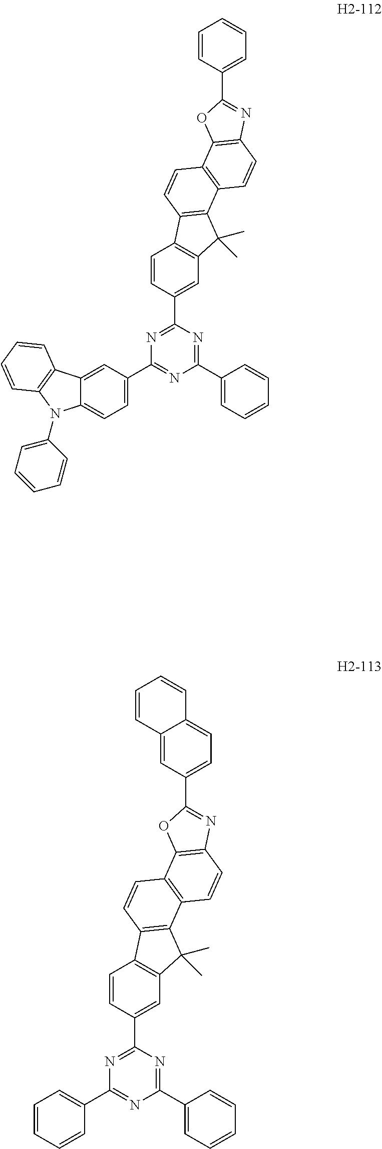 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)oc43)c21 ; CC1(C)c2cc(-c3nc(-c4ccccc4)nc(-c4ccccc4)n3)ccc2-c2ccc3c(ccc4nc(-c5ccc6ccccc6c5)oc43)c21]

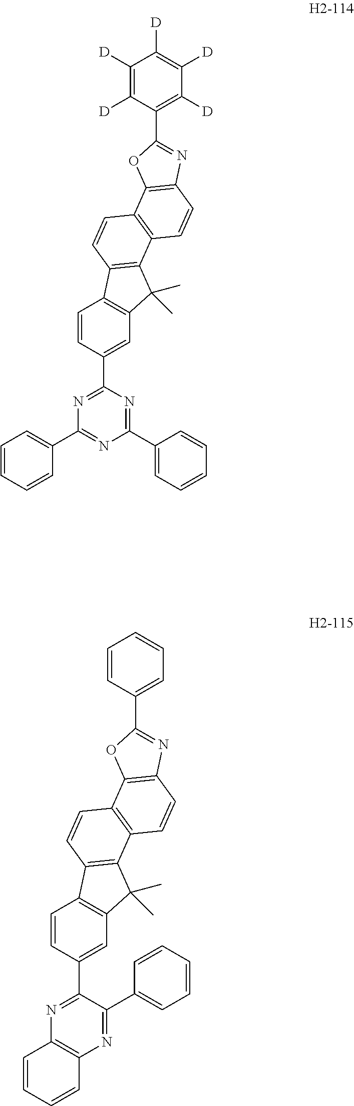 [SMILES: [2H]c1c([2H])c([2H])c(-c2nc3ccc4c5c(ccc4c3o2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)oc43)c21]

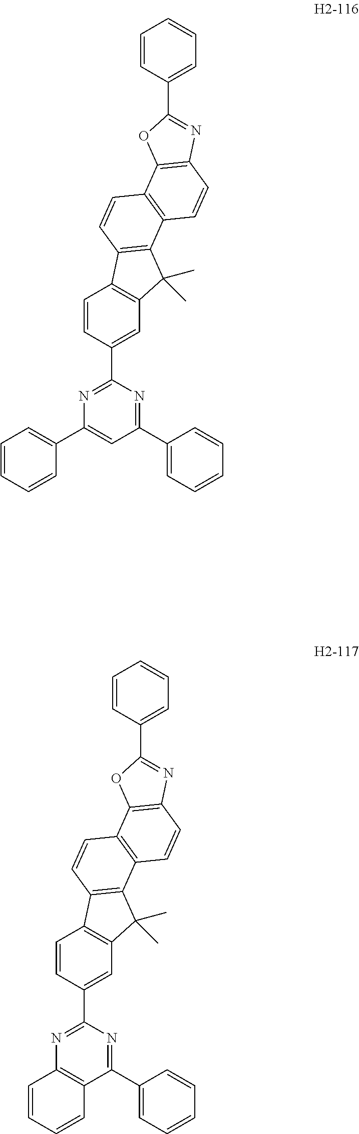 [SMILES: CC1(C)c2cc(-c3nc(-c4ccccc4)cc(-c4ccccc4)n3)ccc2-c2ccc3c(ccc4nc(-c5ccccc5)oc43)c21 ; CC1(C)c2cc(-c3nc(-c4ccccc4)c4ccccc4n3)ccc2-c2ccc3c(ccc4nc(-c5ccccc5)oc43)c21]

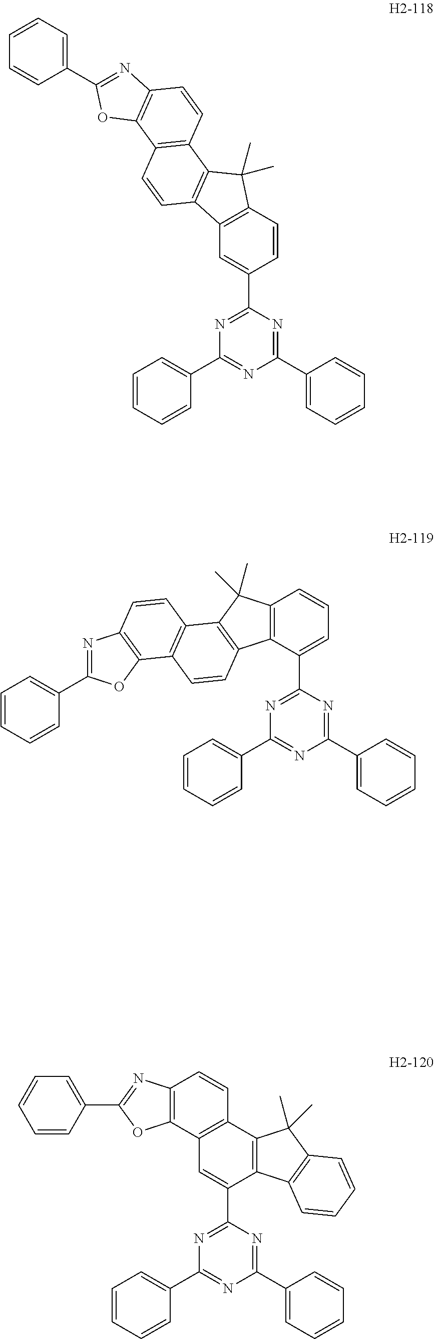 [SMILES: CC1(C)c2ccc(-c3nc(-c4ccccc4)nc(-c4ccccc4)n3)cc2-c2ccc3c(ccc4nc(-c5ccccc5)oc43)c21 ; CC1(C)c2cccc(-c3nc(-c4ccccc4)nc(-c4ccccc4)n3)c2-c2ccc3c(ccc4nc(-c5ccccc5)oc43)c21 ; CC1(C)c2ccccc2-c2c(-c3nc(-c4ccccc4)nc(-c4ccccc4)n3)cc3c(ccc4nc(-c5ccccc5)oc43)c21]

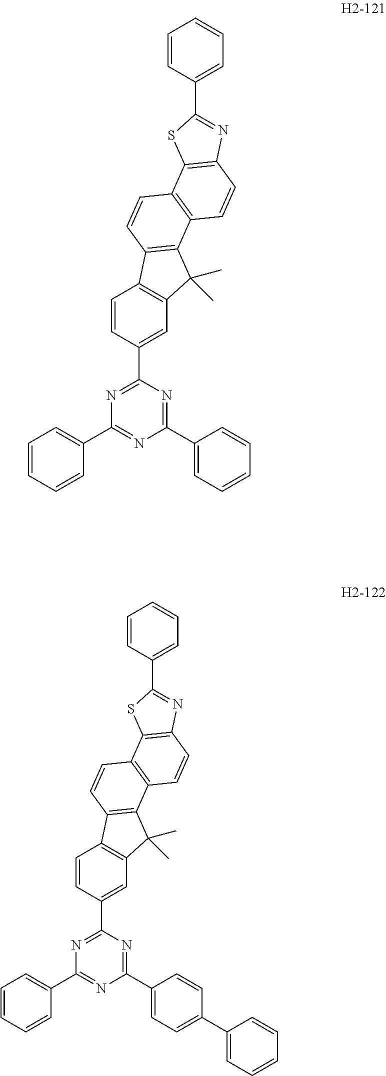 [SMILES: CC1(C)c2cc(-c3nc(-c4ccccc4)nc(-c4ccccc4)n3)ccc2-c2ccc3c(ccc4nc(-c5ccccc5)sc43)c21 ; CC1(C)c2cc(-c3nc(-c4ccccc4)nc(-c4ccc(-c5ccccc5)cc4)n3)ccc2-c2ccc3c(ccc4nc(-c5ccccc5)sc43)c21]

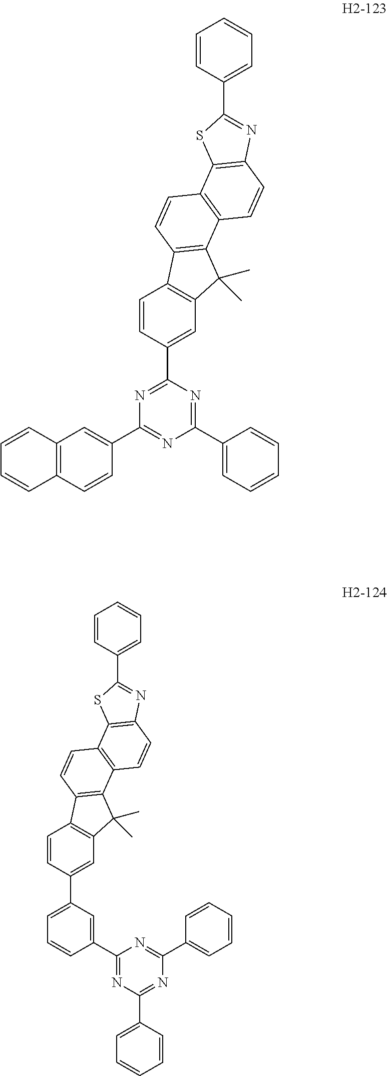 [SMILES: CC1(C)c2cc(-c3nc(-c4ccccc4)nc(-c4ccc5ccccc5c4)n3)ccc2-c2ccc3c(ccc4nc(-c5ccccc5)sc43)c21 ; CC1(C)c2cc(-c3cccc(-c4nc(-c5ccccc5)nc(-c5ccccc5)n4)c3)ccc2-c2ccc3c(ccc4nc(-c5ccccc5)sc43)c21]

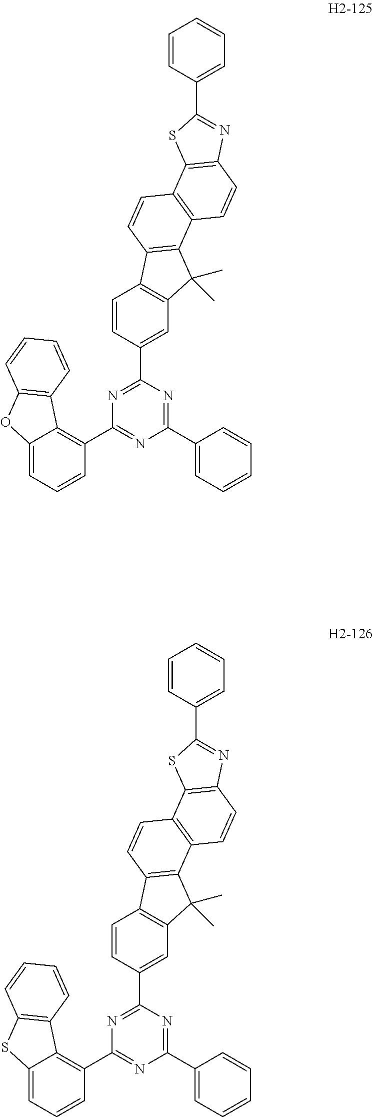 [SMILES: CC1(C)c2cc(-c3nc(-c4ccccc4)nc(-c4cccc5oc6ccccc6c45)n3)ccc2-c2ccc3c(ccc4nc(-c5ccccc5)sc43)c21 ; CC1(C)c2cc(-c3nc(-c4ccccc4)nc(-c4cccc5sc6ccccc6c45)n3)ccc2-c2ccc3c(ccc4nc(-c5ccccc5)sc43)c21]

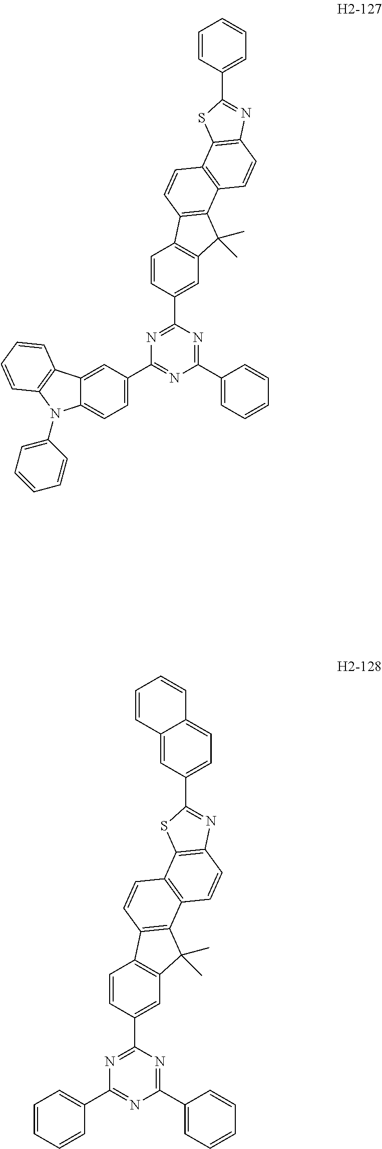 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)sc43)c21 ; CC1(C)c2cc(-c3nc(-c4ccccc4)nc(-c4ccccc4)n3)ccc2-c2ccc3c(ccc4nc(-c5ccc6ccccc6c5)sc43)c21]

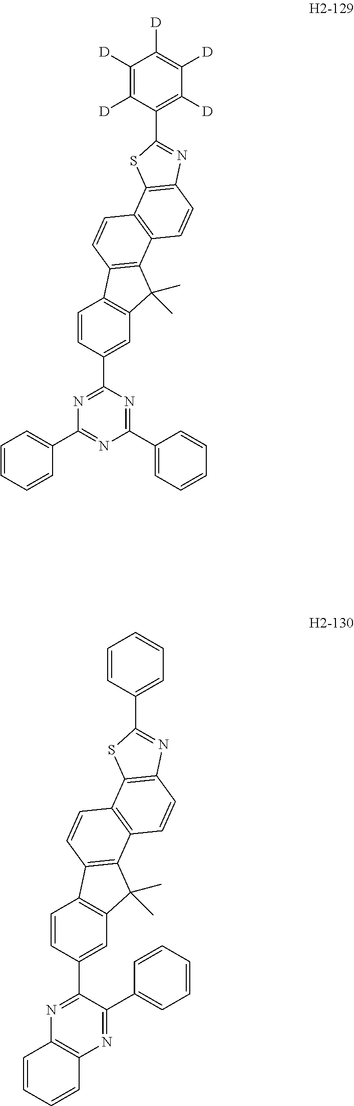 [SMILES: [2H]c1c([2H])c([2H])c(-c2nc3ccc4c5c(ccc4c3s2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)sc43)c21]

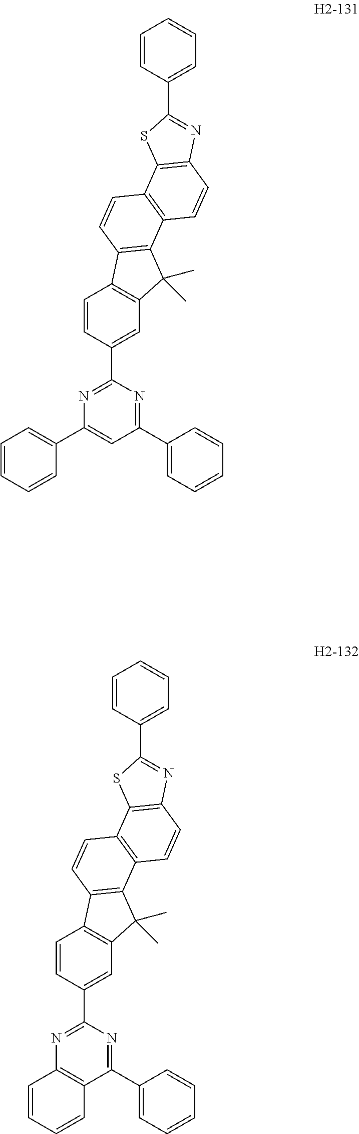 [SMILES: CC1(C)c2cc(-c3nc(-c4ccccc4)cc(-c4ccccc4)n3)ccc2-c2ccc3c(ccc4nc(-c5ccccc5)sc43)c21 ; CC1(C)c2cc(-c3nc(-c4ccccc4)c4ccccc4n3)ccc2-c2ccc3c(ccc4nc(-c5ccccc5)sc43)c21]

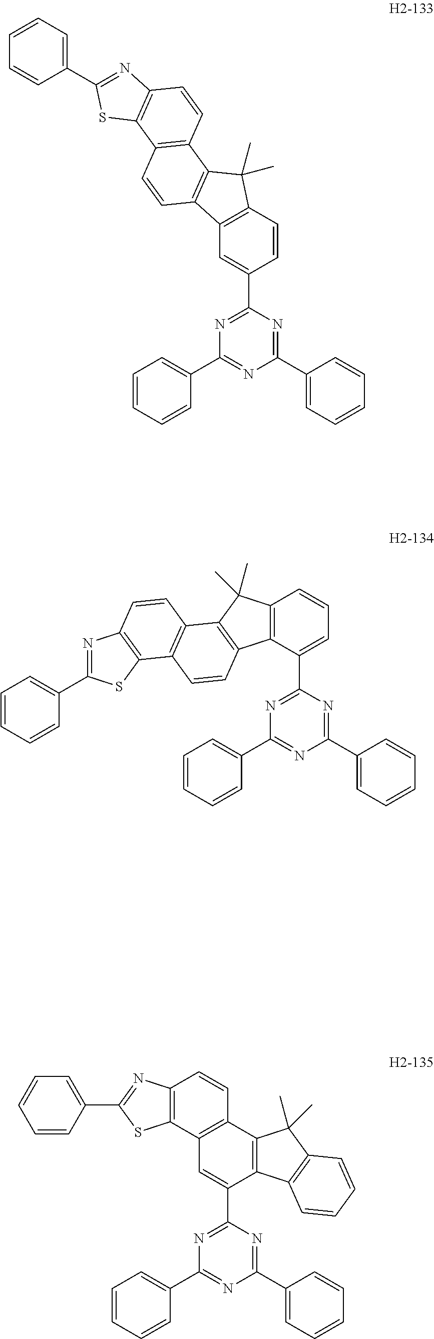 [SMILES: CC1(C)c2ccc(-c3nc(-c4ccccc4)nc(-c4ccccc4)n3)cc2-c2ccc3c(ccc4nc(-c5ccccc5)sc43)c21 ; CC1(C)c2cccc(-c3nc(-c4ccccc4)nc(-c4ccccc4)n3)c2-c2ccc3c(ccc4nc(-c5ccccc5)sc43)c21 ; CC1(C)c2ccccc2-c2c(-c3nc(-c4ccccc4)nc(-c4ccccc4)n3)cc3c(ccc4nc(-c5ccccc5)sc43)c21]

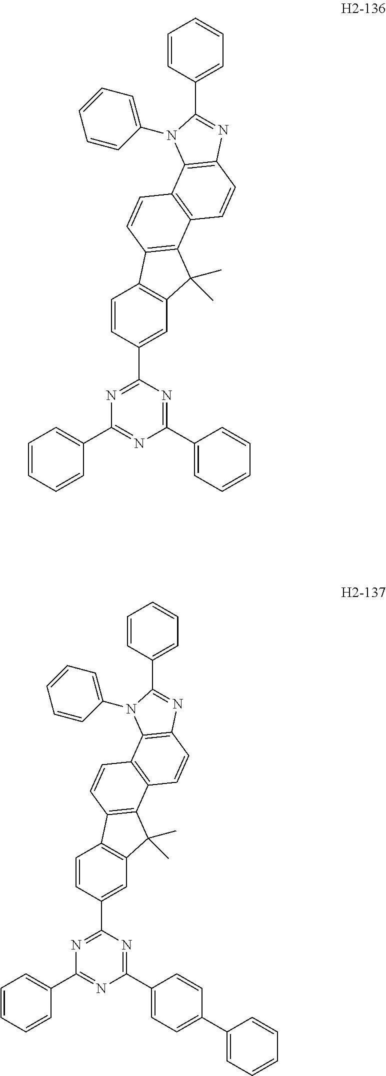 [SMILES: CC1(C)c2cc(-c3nc(-c4ccccc4)nc(-c4ccccc4)n3)ccc2-c2ccc3c(ccc4nc(-c5ccccc5)n(-c5ccccc5)c43)c21 ; CC1(C)c2cc(-c3nc(-c4ccccc4)nc(-c4ccc(-c5ccccc5)cc4)n3)ccc2-c2ccc3c(ccc4nc(-c5ccccc5)n(-c5ccccc5)c43)c21]

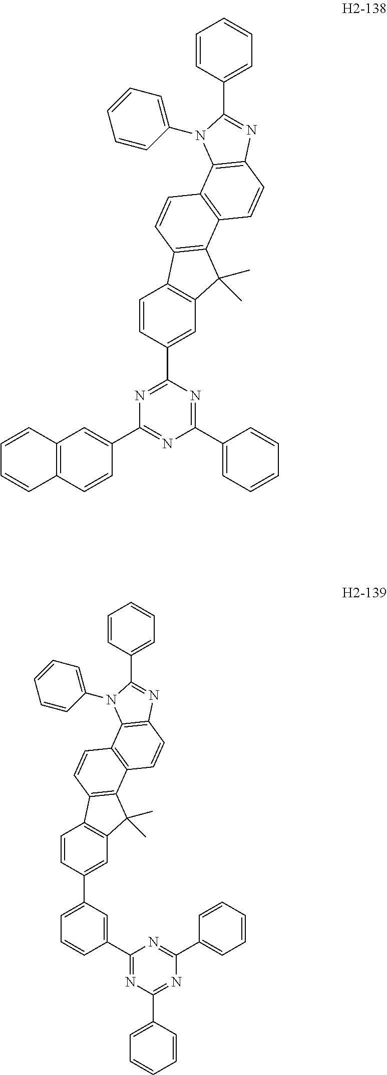 [SMILES: CC1(C)c2cc(-c3nc(-c4ccccc4)nc(-c4ccc5ccccc5c4)n3)ccc2-c2ccc3c(ccc4nc(-c5ccccc5)n(-c5ccccc5)c43)c21 ; CC1(C)c2cc(-c3cccc(-c4nc(-c5ccccc5)nc(-c5ccccc5)n4)c3)ccc2-c2ccc3c(ccc4nc(-c5ccccc5)n(-c5ccccc5)c43)c21]

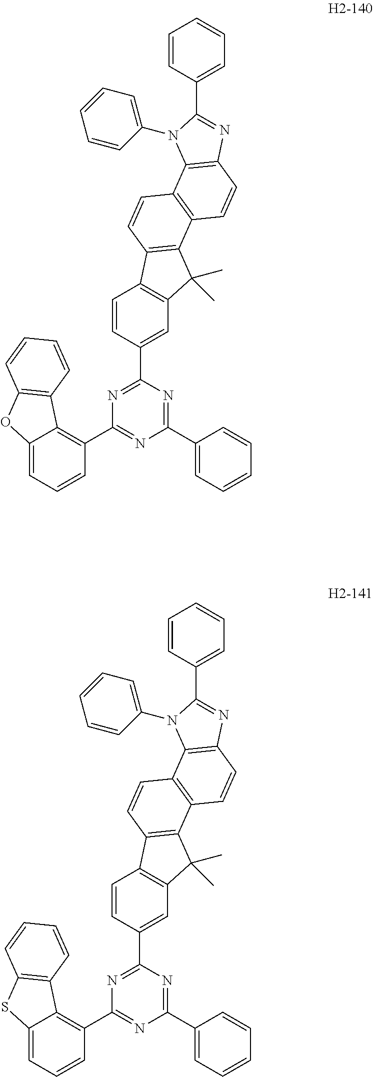 [SMILES: CC1(C)c2cc(-c3nc(-c4ccccc4)nc(-c4cccc5oc6ccccc6c45)n3)ccc2-c2ccc3c(ccc4nc(-c5ccccc5)n(-c5ccccc5)c43)c21 ; CC1(C)c2cc(-c3nc(-c4ccccc4)nc(-c4cccc5sc6ccccc6c45)n3)ccc2-c2ccc3c(ccc4nc(-c5ccccc5)n(-c5ccccc5)c43)c21]

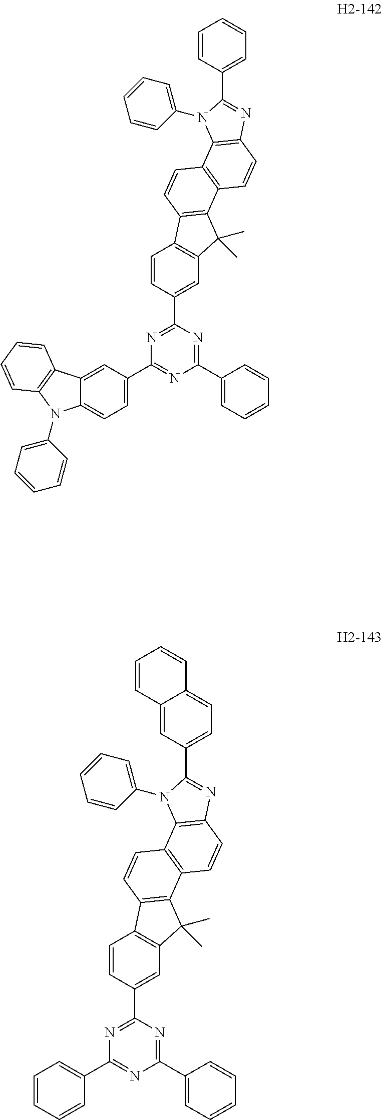 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)n(-c5ccccc5)c43)c21 ; CC1(C)c2cc(-c3nc(-c4ccccc4)nc(-c4ccccc4)n3)ccc2-c2ccc3c(ccc4nc(-c5ccc6ccccc6c5)n(-c5ccccc5)c43)c21]

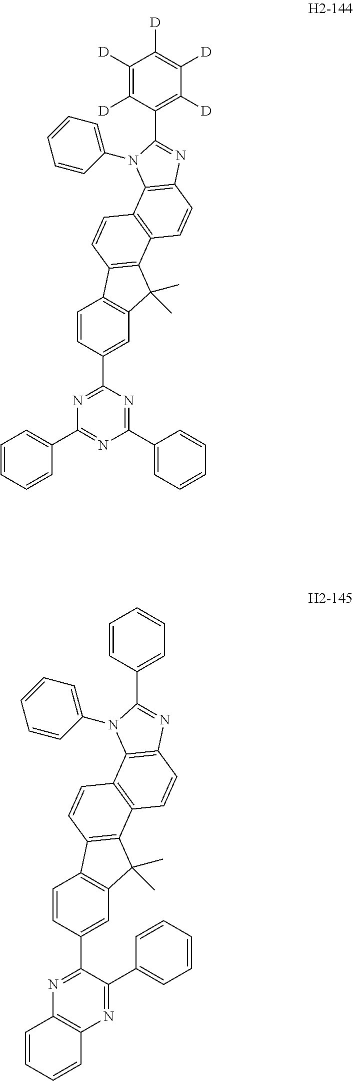 [SMILES: [2H]c1c([2H])c([2H])c(-c2nc3ccc4c5c(ccc4c3n2-c2ccccc2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)n(-c5ccccc5)c43)c21]

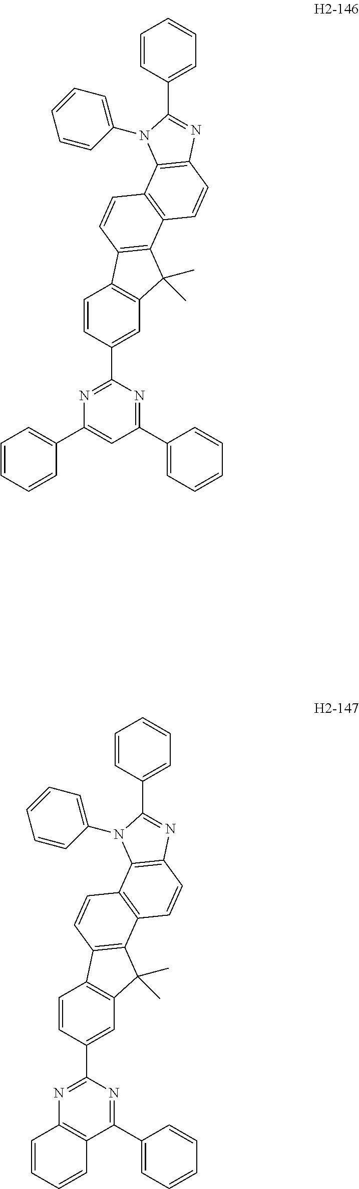 [SMILES: CC1(C)c2cc(-c3nc(-c4ccccc4)cc(-c4ccccc4)n3)ccc2-c2ccc3c(ccc4nc(-c5ccccc5)n(-c5ccccc5)c43)c21 ; CC1(C)c2cc(-c3nc(-c4ccccc4)c4ccccc4n3)ccc2-c2ccc3c(ccc4nc(-c5ccccc5)n(-c5ccccc5)c43)c21]

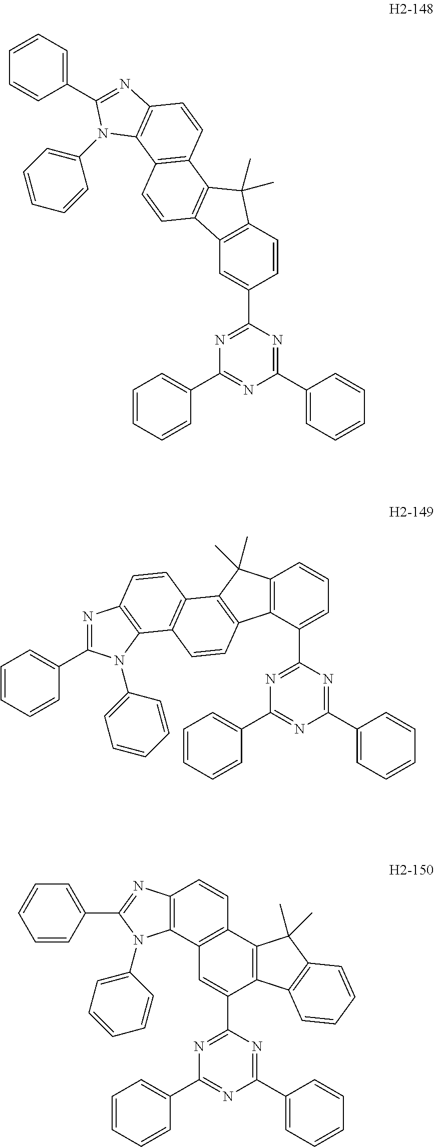 [SMILES: CC1(C)c2ccc(-c3nc(-c4ccccc4)nc(-c4ccccc4)n3)cc2-c2ccc3c(ccc4nc(-c5ccccc5)n(-c5ccccc5)c43)c21 ; CC1(C)c2cccc(-c3nc(-c4ccccc4)nc(-c4ccccc4)n3)c2-c2ccc3c(ccc4nc(-c5ccccc5)n(-c5ccccc5)c43)c21 ; CC1(C)c2ccccc2-c2c(-c3nc(-c4ccccc4)nc(-c4ccccc4)n3)cc3c(ccc4nc(-c5ccccc5)n(-c5ccccc5)c43)c21]

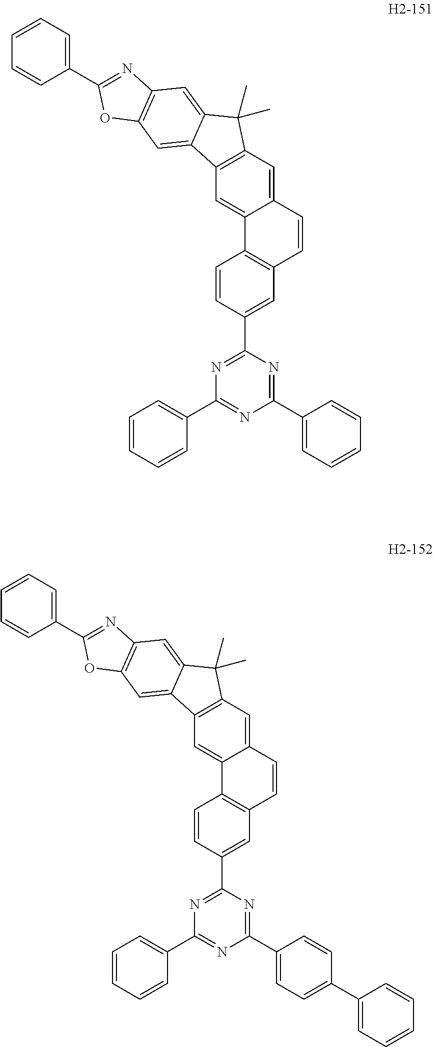 [SMILES: CC1(C)c2cc3ccc4cc(-c5nc(-c6ccccc6)nc(-c6ccccc6)n5)ccc4c3cc2-c2cc3oc(-c4ccccc4)nc3cc21 ; CC1(C)c2cc3ccc4cc(-c5nc(-c6ccccc6)nc(-c6ccc(-c7ccccc7)cc6)n5)ccc4c3cc2-c2cc3oc(-c4ccccc4)nc3cc21]

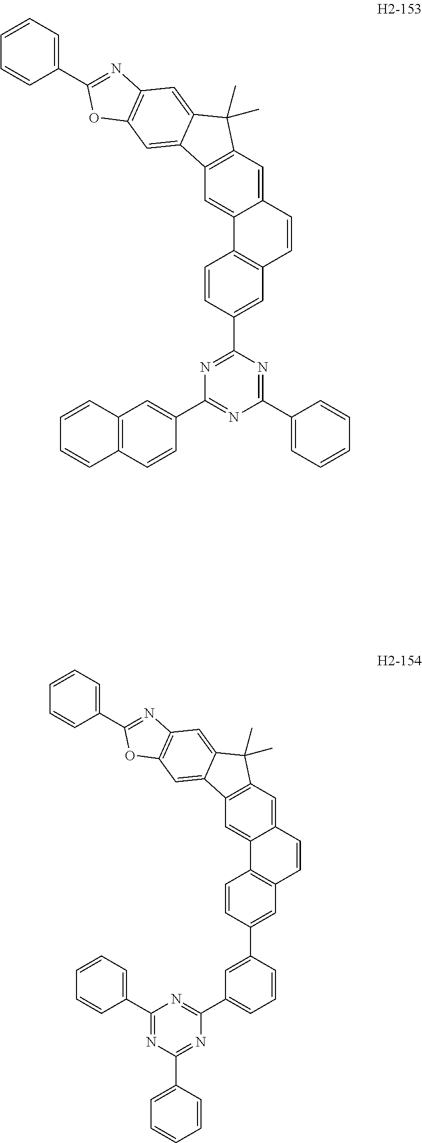 [SMILES: CC1(C)c2cc3ccc4cc(-c5nc(-c6ccccc6)nc(-c6ccc7ccccc7c6)n5)ccc4c3cc2-c2cc3oc(-c4ccccc4)nc3cc21 ; CC1(C)c2cc3ccc4cc(-c5cccc(-c6nc(-c7ccccc7)nc(-c7ccccc7)n6)c5)ccc4c3cc2-c2cc3oc(-c4ccccc4)nc3cc21]

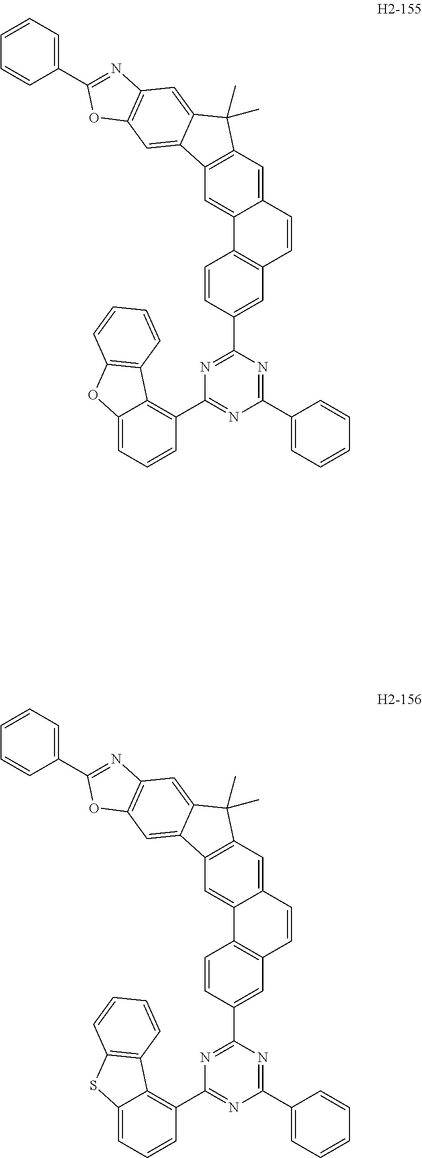 [SMILES: CC1(C)c2cc3ccc4cc(-c5nc(-c6ccccc6)nc(-c6cccc7oc8ccccc8c67)n5)ccc4c3cc2-c2cc3oc(-c4ccccc4)nc3cc21 ; CC1(C)c2cc3ccc4cc(-c5nc(-c6ccccc6)nc(-c6cccc7sc8ccccc8c67)n5)ccc4c3cc2-c2cc3oc(-c4ccccc4)nc3cc21]

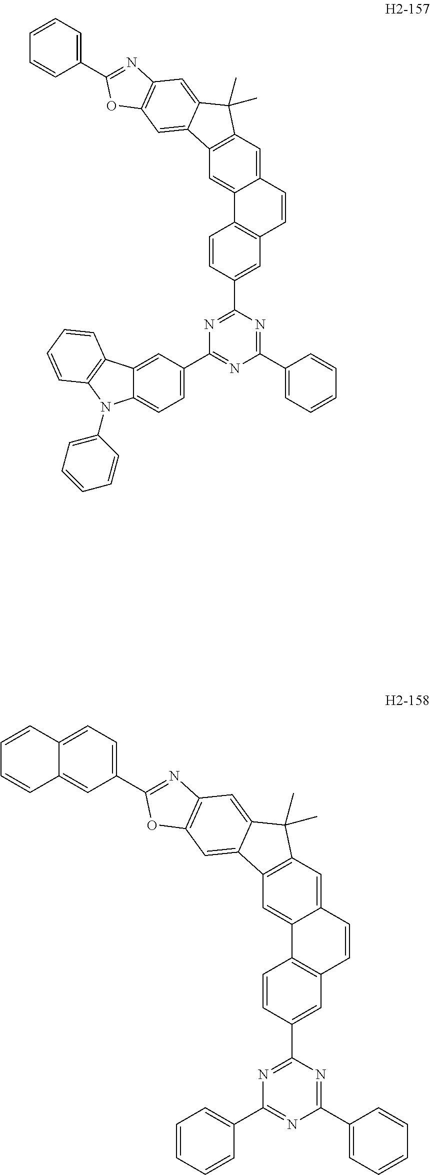 [SMILES: CC1(C)c2cc3ccc4cc(-c5nc(-c6ccccc6)nc(-c6ccc7c(c6)c6ccccc6n7-c6ccccc6)n5)ccc4c3cc2-c2cc3oc(-c4ccccc4)nc3cc21 ; CC1(C)c2cc3ccc4cc(-c5nc(-c6ccccc6)nc(-c6ccccc6)n5)ccc4c3cc2-c2cc3oc(-c4ccc5ccccc5c4)nc3cc21]

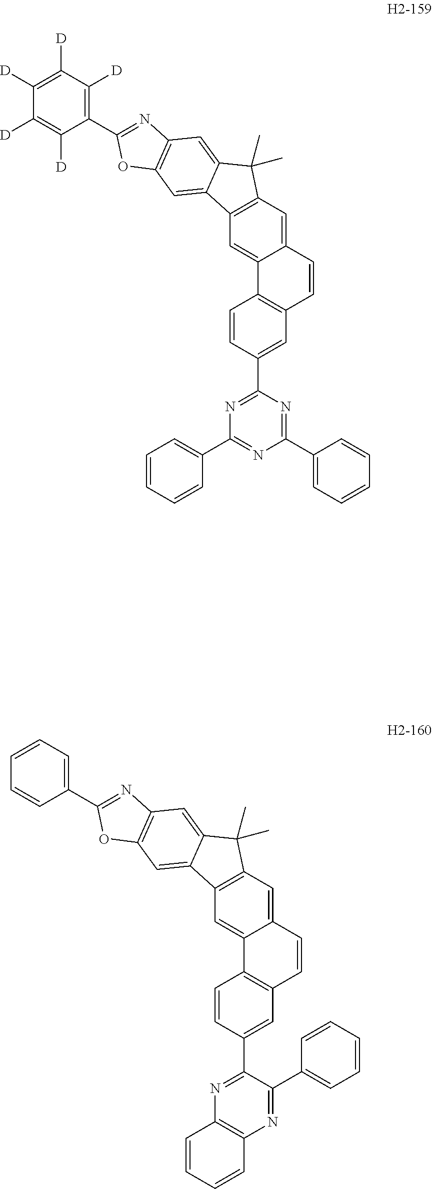 [SMILES: [2H]c1c([2H])c([2H])c(-c2nc3cc4c(cc3o2)-c2cc3c(ccc5cc(-c6nc(-c7ccccc7)nc(-c7ccccc7)n6)ccc53)cc2C4(C)C)c([2H])c1[2H] ; CC1(C)c2cc3ccc4cc(-c5nc6ccccc6nc5-c5ccccc5)ccc4c3cc2-c2cc3oc(-c4ccccc4)nc3cc21]

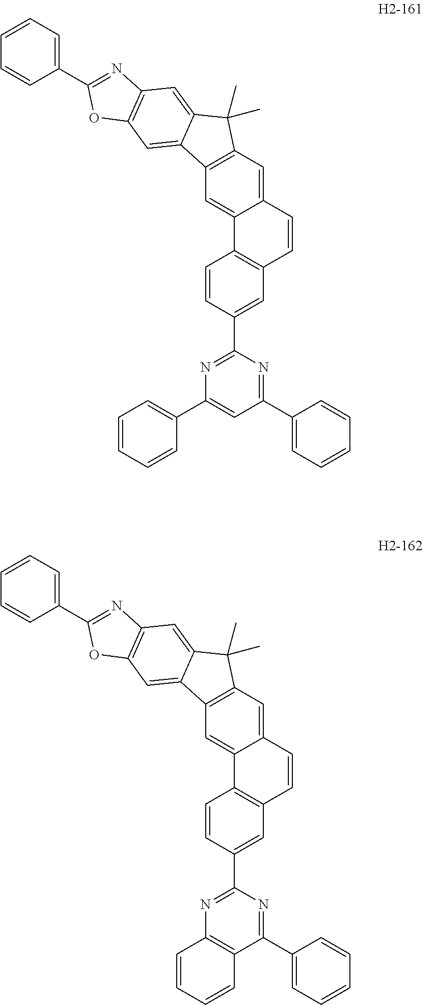 [SMILES: CC1(C)c2cc3ccc4cc(-c5nc(-c6ccccc6)cc(-c6ccccc6)n5)ccc4c3cc2-c2cc3oc(-c4ccccc4)nc3cc21 ; CC1(C)c2cc3ccc4cc(-c5nc(-c6ccccc6)c6ccccc6n5)ccc4c3cc2-c2cc3oc(-c4ccccc4)nc3cc21]

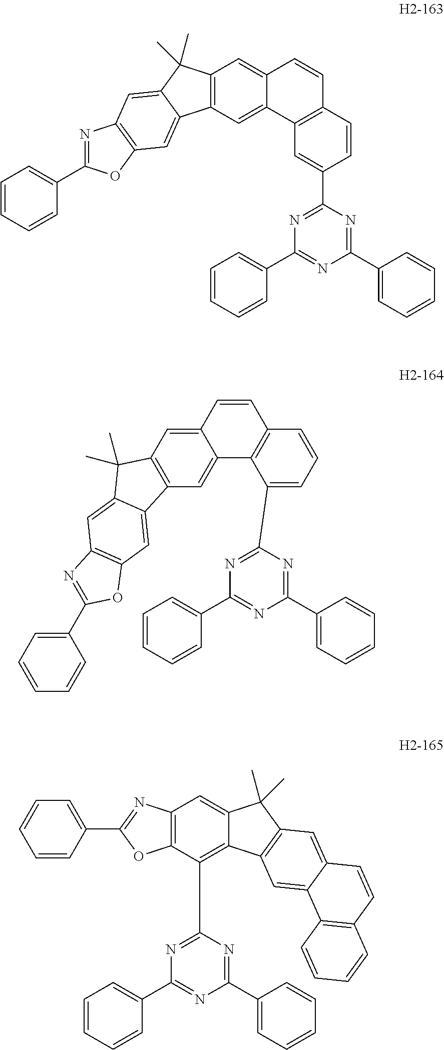 [SMILES: CC1(C)c2cc3ccc4ccc(-c5nc(-c6ccccc6)nc(-c6ccccc6)n5)cc4c3cc2-c2cc3oc(-c4ccccc4)nc3cc21 ; CC1(C)c2cc3ccc4cccc(-c5nc(-c6ccccc6)nc(-c6ccccc6)n5)c4c3cc2-c2cc3oc(-c4ccccc4)nc3cc21 ; CC1(C)c2cc3ccc4ccccc4c3cc2-c2c1cc1nc(-c3ccccc3)oc1c2-c1nc(-c2ccccc2)nc(-c2ccccc2)n1]

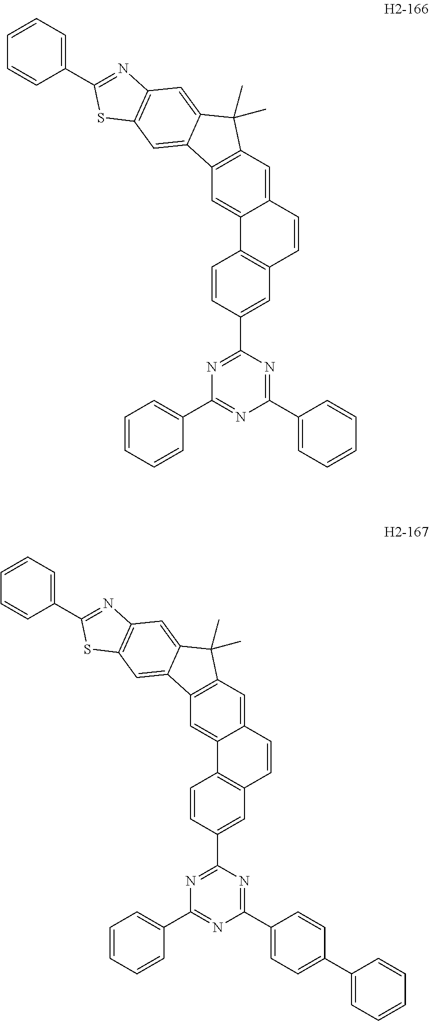 [SMILES: CC1(C)c2cc3ccc4cc(-c5nc(-c6ccccc6)nc(-c6ccccc6)n5)ccc4c3cc2-c2cc3sc(-c4ccccc4)nc3cc21 ; CC1(C)c2cc3ccc4cc(-c5nc(-c6ccccc6)nc(-c6ccc(-c7ccccc7)cc6)n5)ccc4c3cc2-c2cc3sc(-c4ccccc4)nc3cc21]

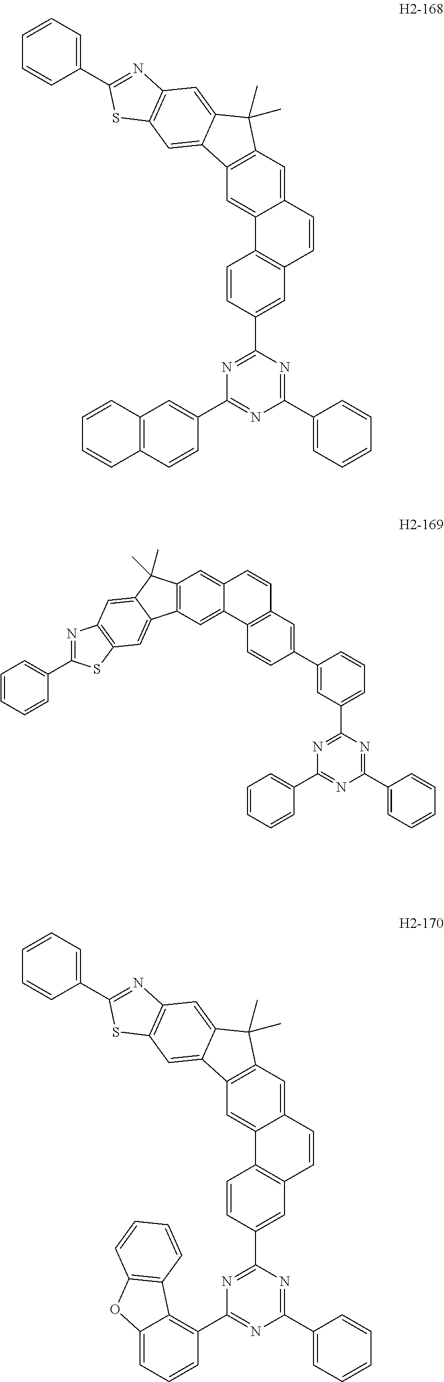 [SMILES: CC1(C)c2cc3ccc4cc(-c5nc(-c6ccccc6)nc(-c6ccc7ccccc7c6)n5)ccc4c3cc2-c2cc3sc(-c4ccccc4)nc3cc21 ; CC1(C)c2cc3ccc4cc(-c5cccc(-c6nc(-c7ccccc7)nc(-c7ccccc7)n6)c5)ccc4c3cc2-c2cc3sc(-c4ccccc4)nc3cc21 ; CC1(C)c2cc3ccc4cc(-c5nc(-c6ccccc6)nc(-c6cccc7oc8ccccc8c67)n5)ccc4c3cc2-c2cc3sc(-c4ccccc4)nc3cc21]

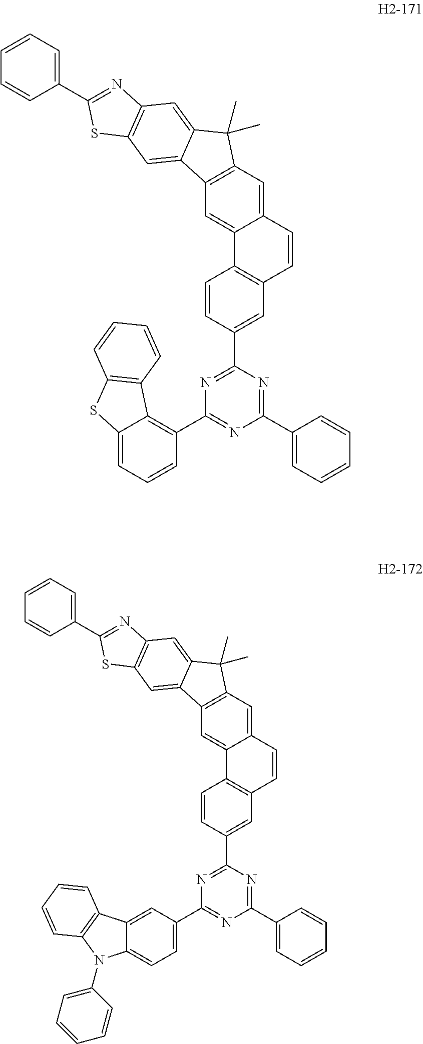 [SMILES: CC1(C)c2cc3ccc4cc(-c5nc(-c6ccccc6)nc(-c6cccc7sc8ccccc8c67)n5)ccc4c3cc2-c2cc3sc(-c4ccccc4)nc3cc21 ; CC1(C)c2cc3ccc4cc(-c5nc(-c6ccccc6)nc(-c6ccc7c(c6)c6ccccc6n7-c6ccccc6)n5)ccc4c3cc2-c2cc3sc(-c4ccccc4)nc3cc21]

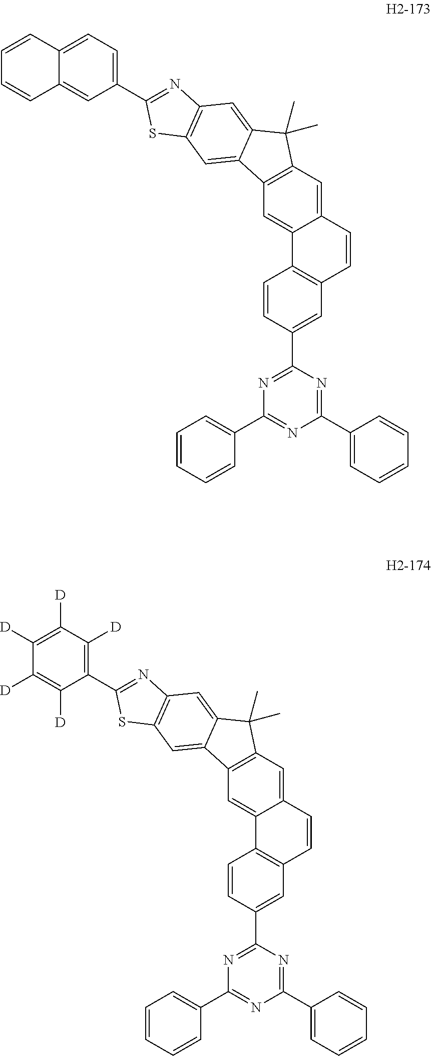 [SMILES: CC1(C)c2cc3ccc4cc(-c5nc(-c6ccccc6)nc(-c6ccccc6)n5)ccc4c3cc2-c2cc3sc(-c4ccc5ccccc5c4)nc3cc21 ; [2H]c1c([2H])c([2H])c(-c2nc3cc4c(cc3s2)-c2cc3c(ccc5cc(-c6nc(-c7ccccc7)nc(-c7ccccc7)n6)ccc53)cc2C4(C)C)c([2H])c1[2H]]

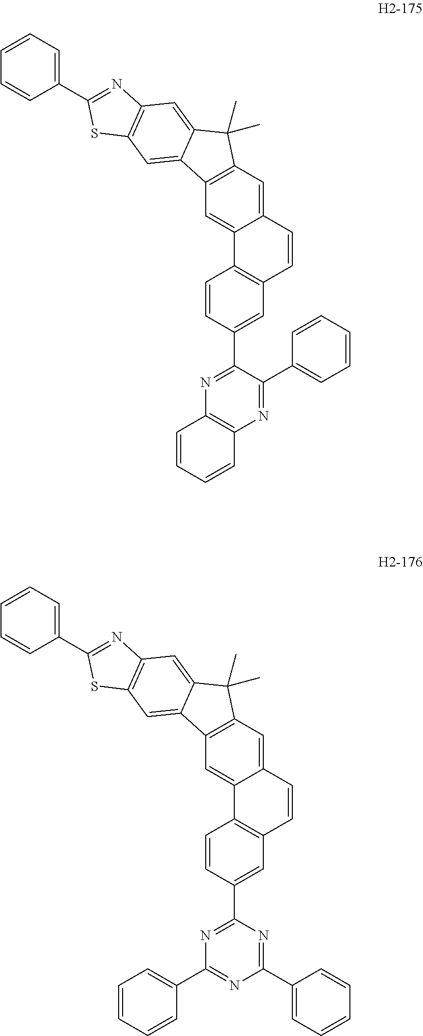 [SMILES: CC1(C)c2cc3ccc4cc(-c5nc6ccccc6nc5-c5ccccc5)ccc4c3cc2-c2cc3sc(-c4ccccc4)nc3cc21 ; CC1(C)c2cc3ccc4cc(-c5nc(-c6ccccc6)nc(-c6ccccc6)n5)ccc4c3cc2-c2cc3sc(-c4ccccc4)nc3cc21]

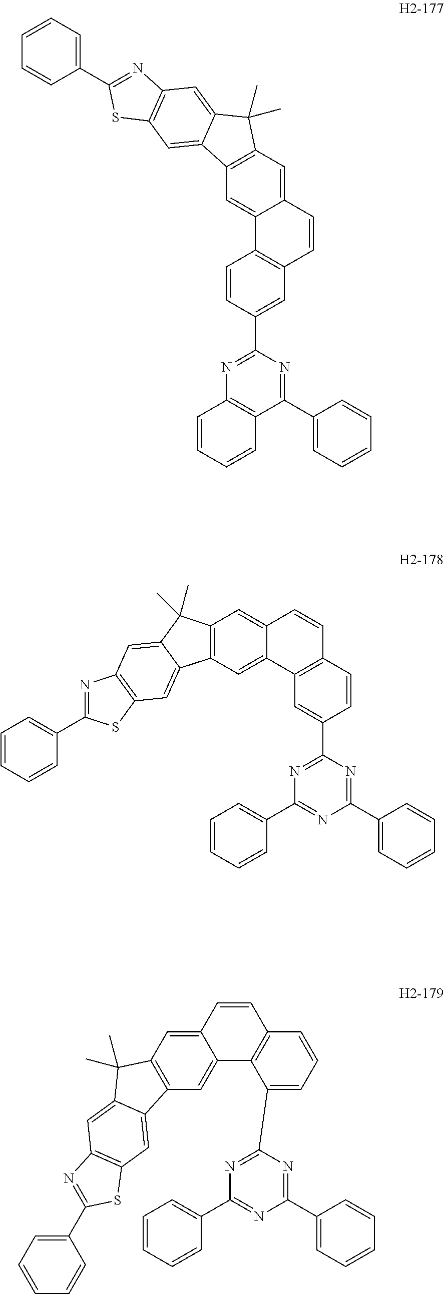 [SMILES: CC1(C)c2cc3ccc4cc(-c5nc(-c6ccccc6)c6ccccc6n5)ccc4c3cc2-c2cc3sc(-c4ccccc4)nc3cc21 ; CC1(C)c2cc3ccc4ccc(-c5nc(-c6ccccc6)nc(-c6ccccc6)n5)cc4c3cc2-c2cc3sc(-c4ccccc4)nc3cc21 ; CC1(C)c2cc3ccc4cccc(-c5nc(-c6ccccc6)nc(-c6ccccc6)n5)c4c3cc2-c2cc3sc(-c4ccccc4)nc3cc21]

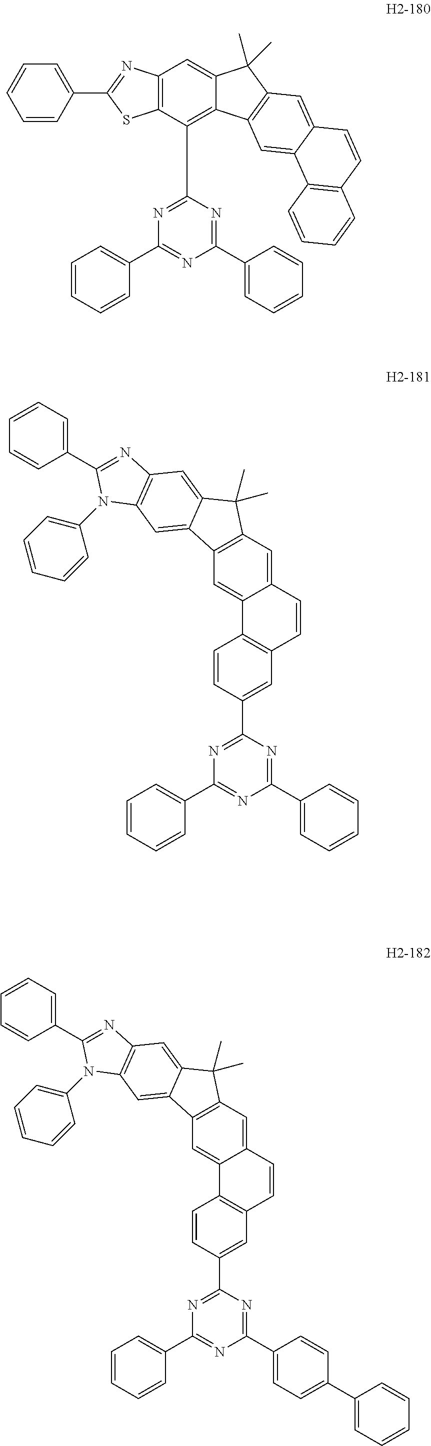 [SMILES: CC1(C)c2cc3ccc4ccccc4c3cc2-c2c1cc1nc(-c3ccccc3)sc1c2-c1nc(-c2ccccc2)nc(-c2ccccc2)n1 ; CC1(C)c2cc3ccc4cc(-c5nc(-c6ccccc6)nc(-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccc(-c7ccccc7)cc6)n5)ccc4c3cc2-c2cc3c(cc21)nc(-c1ccccc1)n3-c1ccccc1]

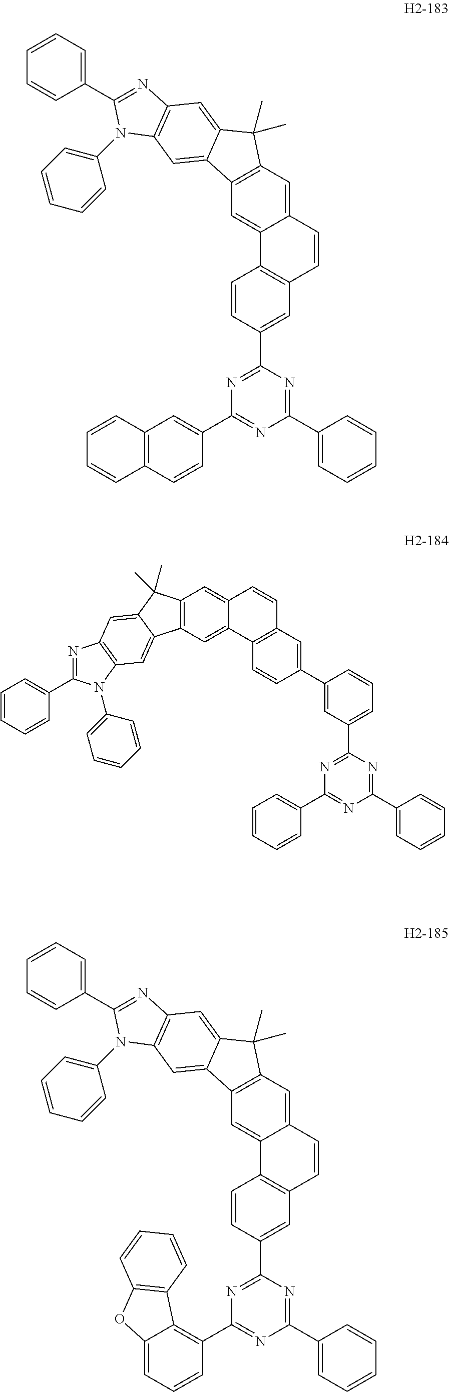 [SMILES: CC1(C)c2cc3ccc4cc(-c5nc(-c6ccccc6)nc(-c6ccc7ccccc7c6)n5)ccc4c3cc2-c2cc3c(cc21)nc(-c1ccccc1)n3-c1ccccc1 ; CC1(C)c2cc3ccc4cc(-c5cccc(-c6nc(-c7ccccc7)nc(-c7ccccc7)n6)c5)ccc4c3cc2-c2cc3c(cc21)nc(-c1ccccc1)n3-c1ccccc1 ; CC1(C)c2cc3ccc4cc(-c5nc(-c6ccccc6)nc(-c6cccc7oc8ccccc8c67)n5)ccc4c3cc2-c2cc3c(cc21)nc(-c1ccccc1)n3-c1ccccc1]

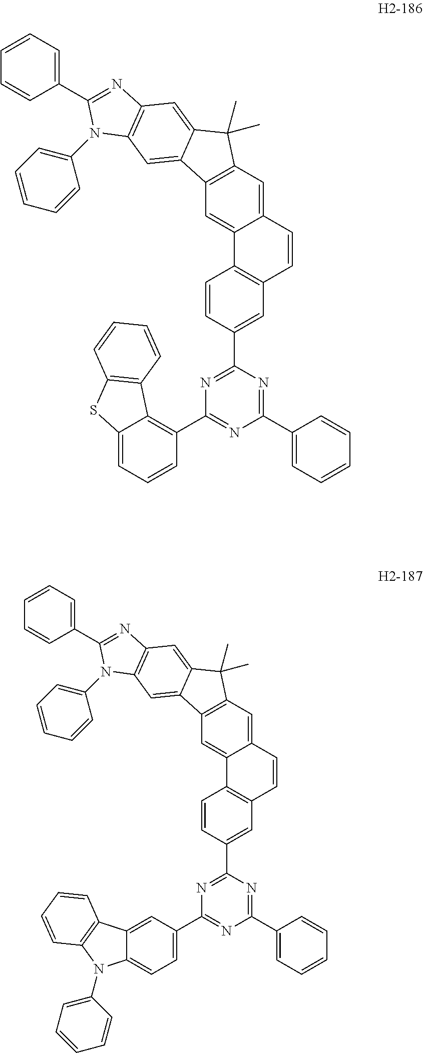 [SMILES: CC1(C)c2cc3ccc4cc(-c5nc(-c6ccccc6)nc(-c6cccc7sc8ccccc8c67)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccc7c(c6)c6ccccc6n7-c6ccccc6)n5)ccc4c3cc2-c2cc3c(cc21)nc(-c1ccccc1)n3-c1ccccc1]

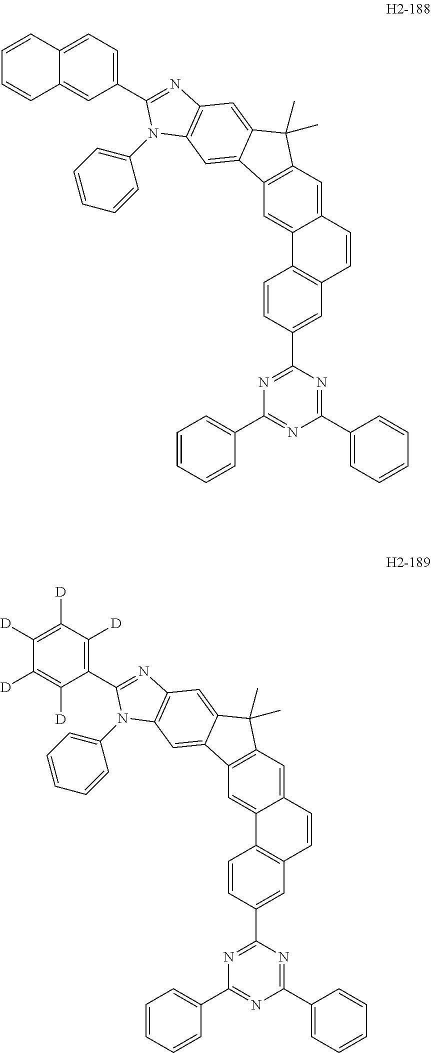 [SMILES: CC1(C)c2cc3ccc4cc(-c5nc(-c6ccccc6)nc(-c6ccccc6)n5)ccc4c3cc2-c2cc3c(cc21)nc(-c1ccc2ccccc2c1)n3-c1ccccc1 ; [2H]c1c([2H])c([2H])c(-c2nc3cc4c(cc3n2-c2ccccc2)-c2cc3c(ccc5cc(-c6nc(-c7ccccc7)nc(-c7ccccc7)n6)ccc53)cc2C4(C)C)c([2H])c1[2H]]

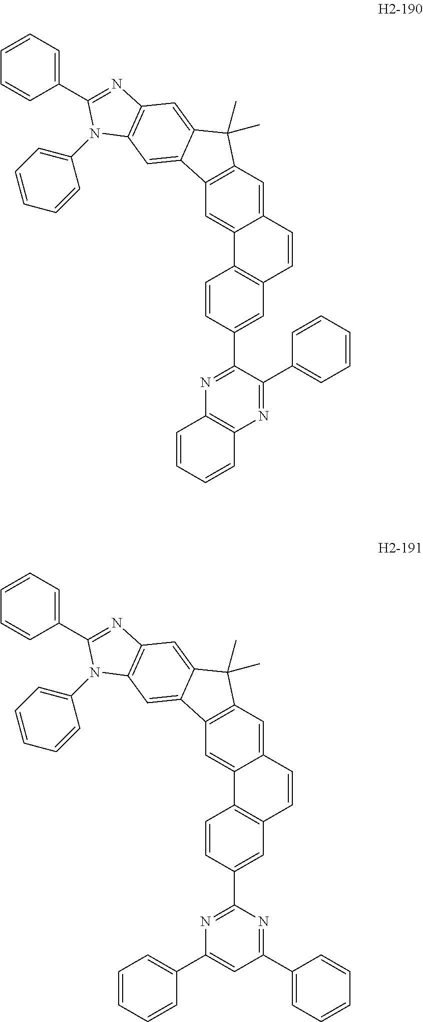 [SMILES: CC1(C)c2cc3ccc4cc(-c5nc6ccccc6nc5-c5ccccc5)ccc4c3cc2-c2cc3c(cc21)nc(-c1ccccc1)n3-c1ccccc1 ; CC1(C)c2cc3ccc4cc(-c5nc(-c6ccccc6)cc(-c6ccccc6)n5)ccc4c3cc2-c2cc3c(cc21)nc(-c1ccccc1)n3-c1ccccc1]

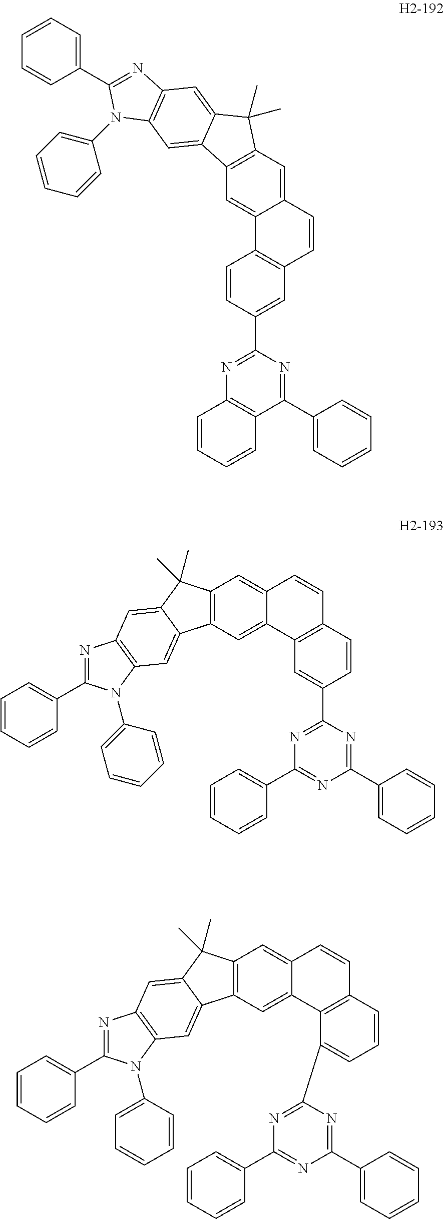 [SMILES: CC1(C)c2cc3ccc4cc(-c5nc(-c6ccccc6)c6ccccc6n5)ccc4c3cc2-c2cc3c(cc21)nc(-c1ccccc1)n3-c1ccccc1 ; CC1(C)c2cc3ccc4ccc(-c5nc(-c6ccccc6)nc(-c6ccccc6)n5)cc4c3cc2-c2cc3c(cc21)nc(-c1ccccc1)n3-c1ccccc1 ; CC1(C)c2cc3ccc4cccc(-c5nc(-c6ccccc6)nc(-c6ccccc6)n5)c4c3cc2-c2cc3c(cc21)nc(-c1ccccc1)n3-c1ccccc1]

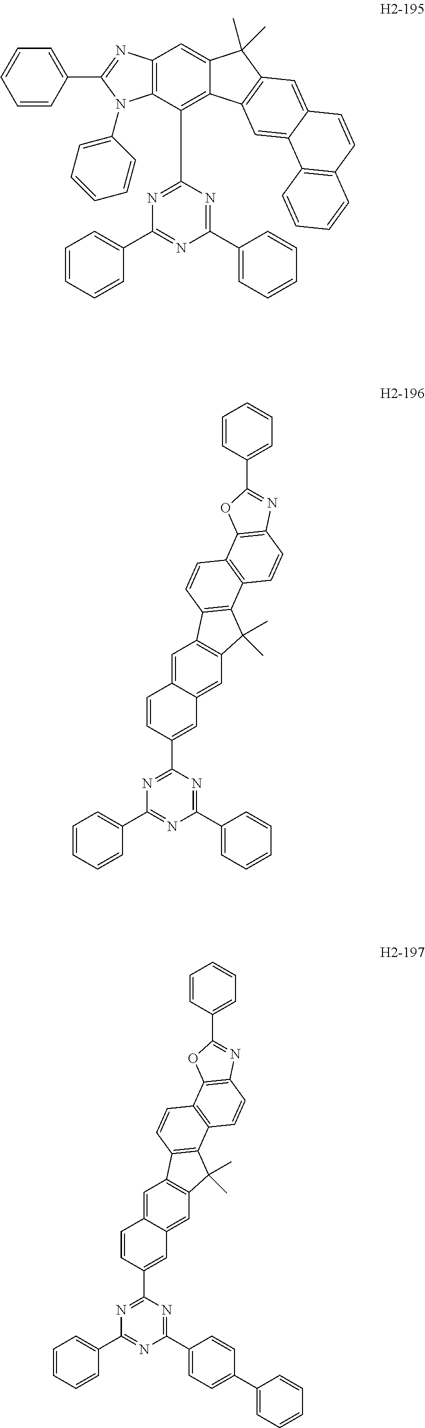 [SMILES: CC1(C)c2cc3ccc4ccccc4c3cc2-c2c1cc1nc(-c3ccccc3)n(-c3ccccc3)c1c2-c1nc(-c2ccccc2)nc(-c2ccccc2)n1 ; CC1(C)c2cc3cc(-c4nc(-c5ccccc5)nc(-c5ccccc5)n4)ccc3cc2-c2ccc3c(ccc4nc(-c5ccccc5)oc43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)oc43)c21]

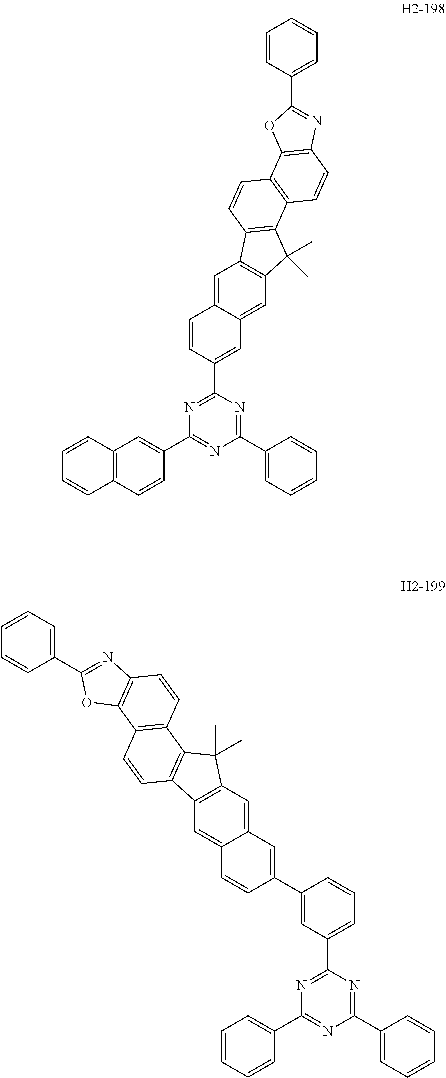 [SMILES: CC1(C)c2cc3cc(-c4nc(-c5ccccc5)nc(-c5ccc6ccccc6c5)n4)ccc3cc2-c2ccc3c(ccc4nc(-c5ccccc5)oc43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)oc43)c21]

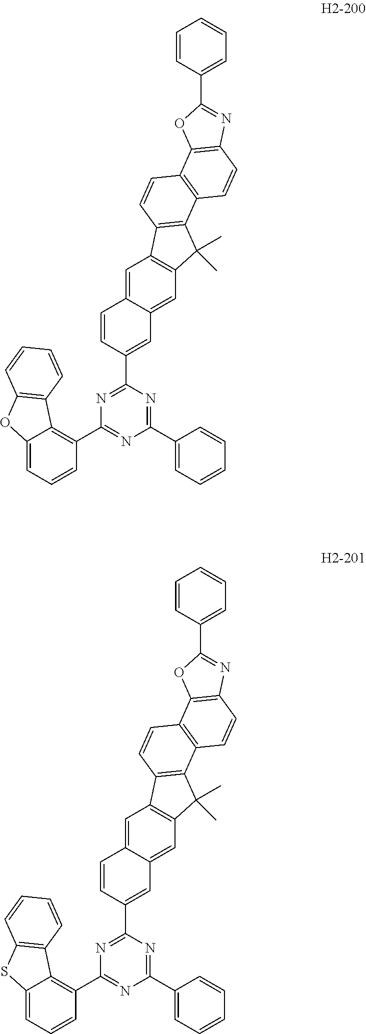 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)oc43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)oc43)c21]

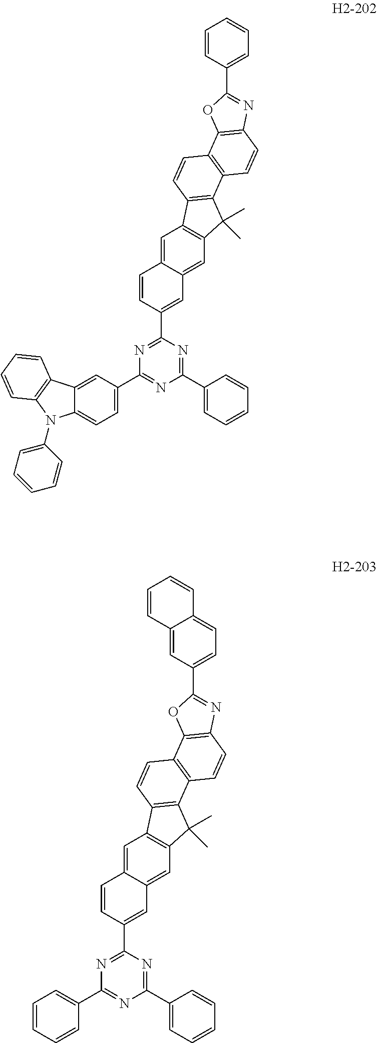 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)oc43)c21 ; CC1(C)c2cc3cc(-c4nc(-c5ccccc5)nc(-c5ccccc5)n4)ccc3cc2-c2ccc3c(ccc4nc(-c5ccc6ccccc6c5)oc43)c21]

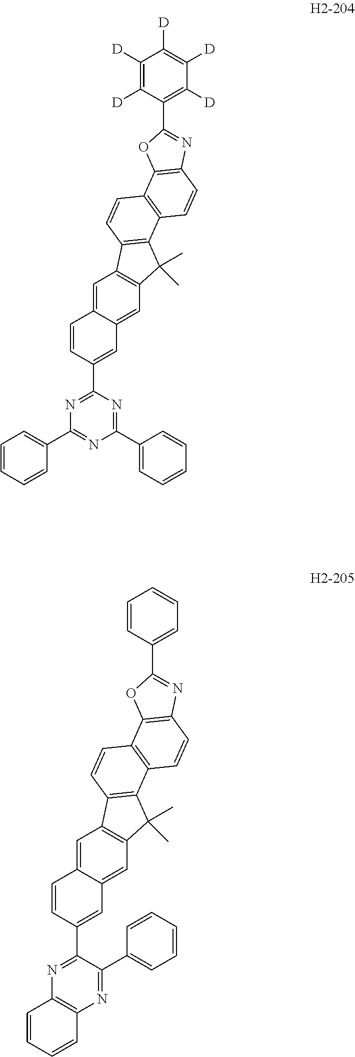 [SMILES: [2H]c1c([2H])c([2H])c(-c2nc3ccc4c5c(ccc4c3o2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)oc43)c21]

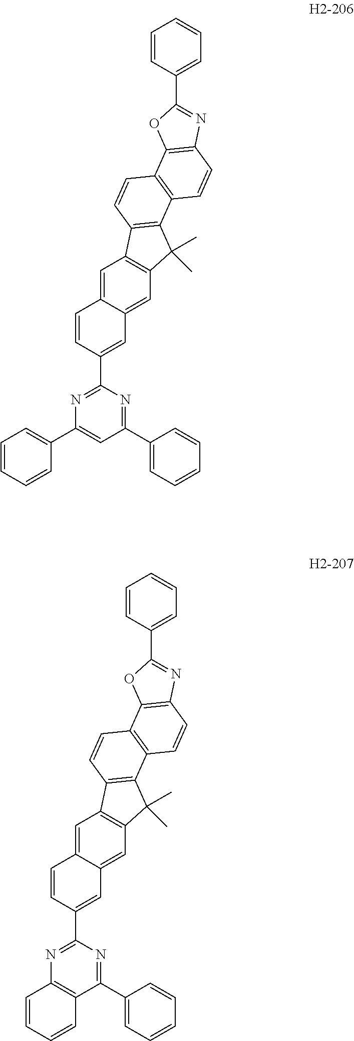 [SMILES: CC1(C)c2cc3cc(-c4nc(-c5ccccc5)cc(-c5ccccc5)n4)ccc3cc2-c2ccc3c(ccc4nc(-c5ccccc5)oc43)c21 ; CC1(C)c2cc3cc(-c4nc(-c5ccccc5)c5ccccc5n4)ccc3cc2-c2ccc3c(ccc4nc(-c5ccccc5)oc43)c21]

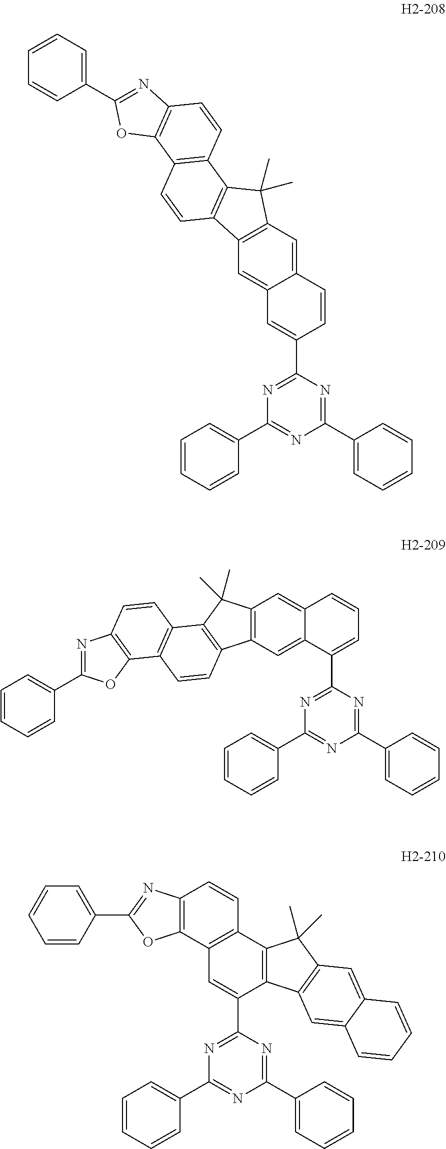 [SMILES: CC1(C)c2cc3ccc(-c4nc(-c5ccccc5)nc(-c5ccccc5)n4)cc3cc2-c2ccc3c(ccc4nc(-c5ccccc5)oc43)c21 ; CC1(C)c2cc3cccc(-c4nc(-c5ccccc5)nc(-c5ccccc5)n4)c3cc2-c2ccc3c(ccc4nc(-c5ccccc5)oc43)c21 ; CC1(C)c2cc3ccccc3cc2-c2c(-c3nc(-c4ccccc4)nc(-c4ccccc4)n3)cc3c(ccc4nc(-c5ccccc5)oc43)c21]

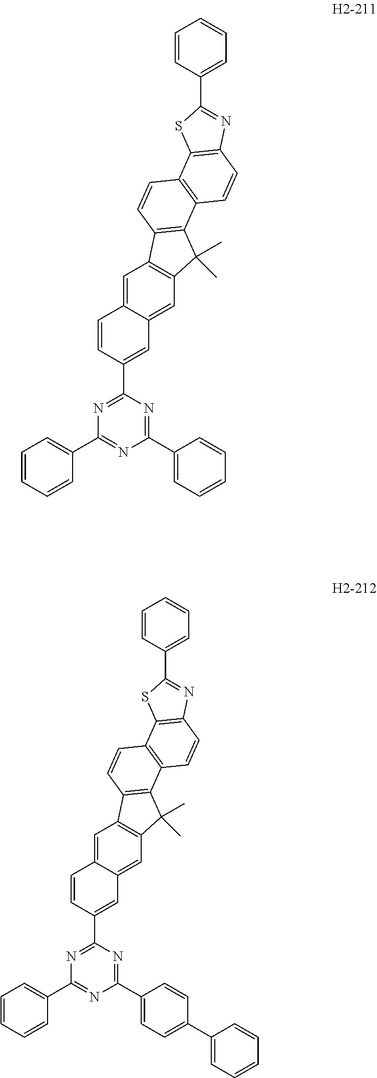 [SMILES: CC1(C)c2cc3cc(-c4nc(-c5ccccc5)nc(-c5ccccc5)n4)ccc3cc2-c2ccc3c(ccc4nc(-c5ccccc5)sc43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)sc43)c21]

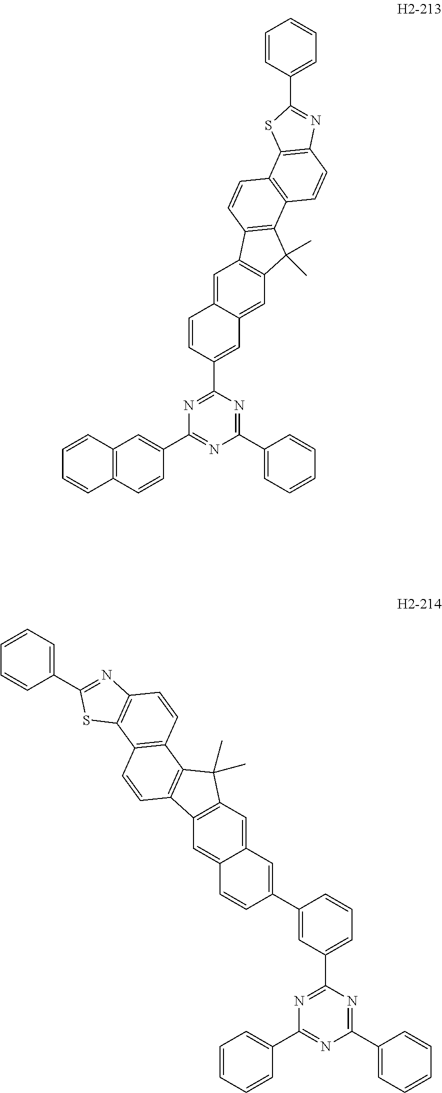 [SMILES: CC1(C)c2cc3cc(-c4nc(-c5ccccc5)nc(-c5ccc6ccccc6c5)n4)ccc3cc2-c2ccc3c(ccc4nc(-c5ccccc5)sc43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)sc43)c21]

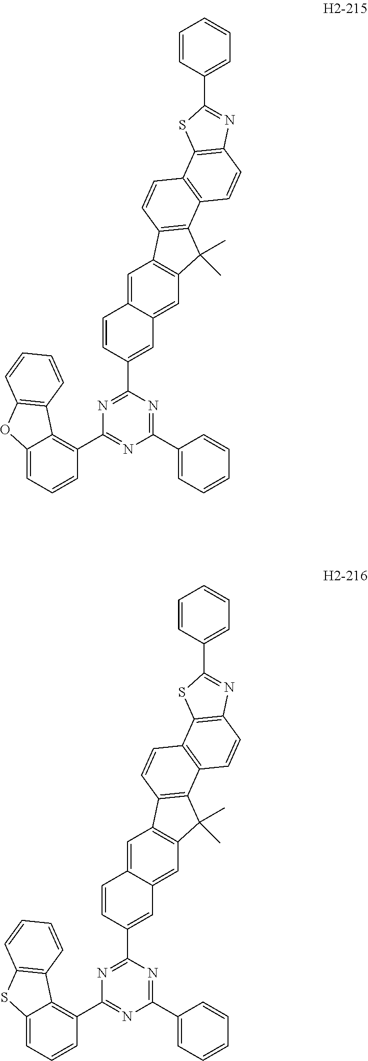 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)sc43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)sc43)c21]

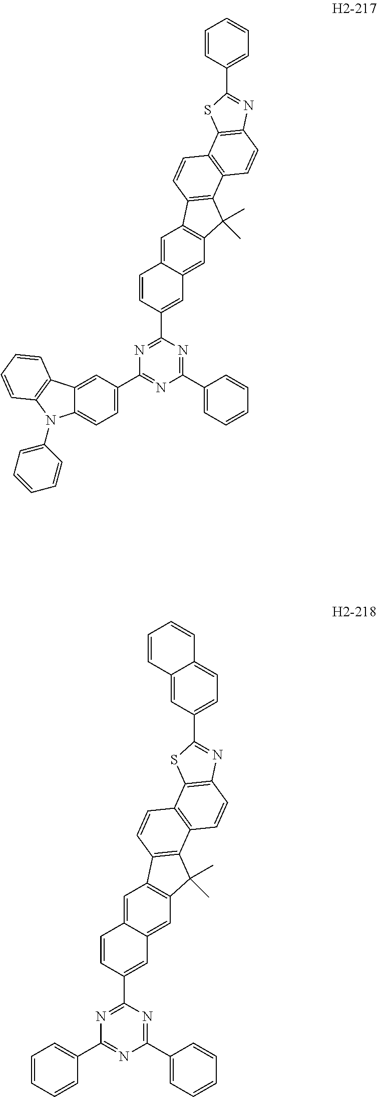 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)sc43)c21 ; CC1(C)c2cc3cc(-c4nc(-c5ccccc5)nc(-c5ccccc5)n4)ccc3cc2-c2ccc3c(ccc4nc(-c5ccc6ccccc6c5)sc43)c21]

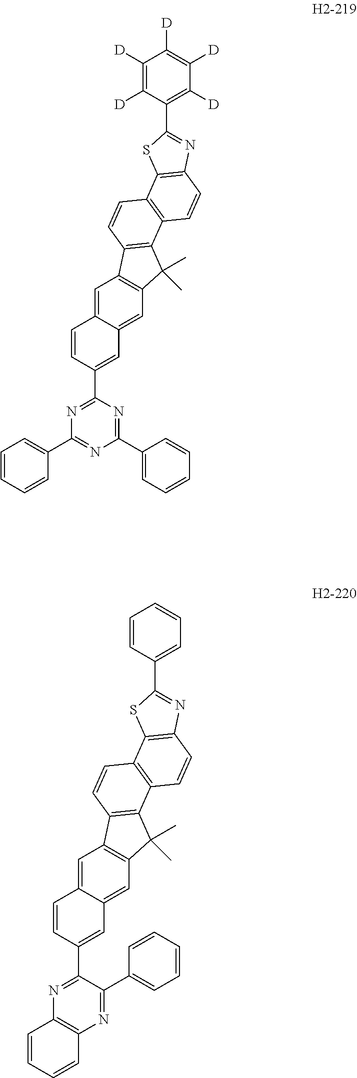 [SMILES: [2H]c1c([2H])c([2H])c(-c2nc3ccc4c5c(ccc4c3s2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)sc43)c21]

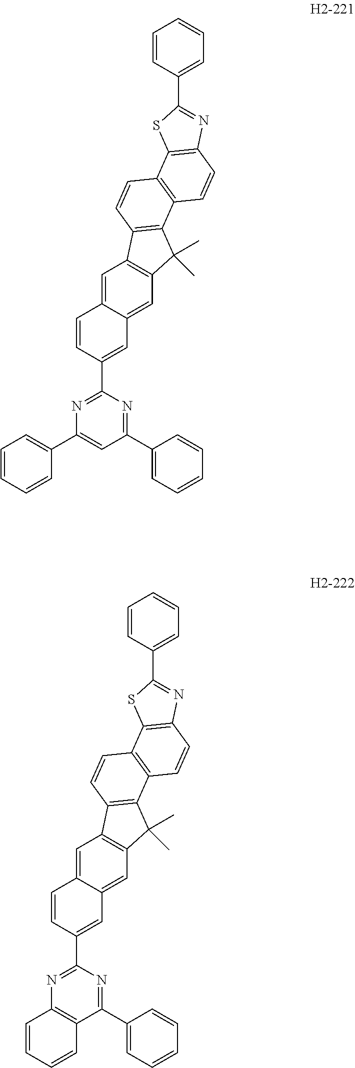 [SMILES: CC1(C)c2cc3cc(-c4nc(-c5ccccc5)cc(-c5ccccc5)n4)ccc3cc2-c2ccc3c(ccc4nc(-c5ccccc5)sc43)c21 ; CC1(C)c2cc3cc(-c4nc(-c5ccccc5)c5ccccc5n4)ccc3cc2-c2ccc3c(ccc4nc(-c5ccccc5)sc43)c21]

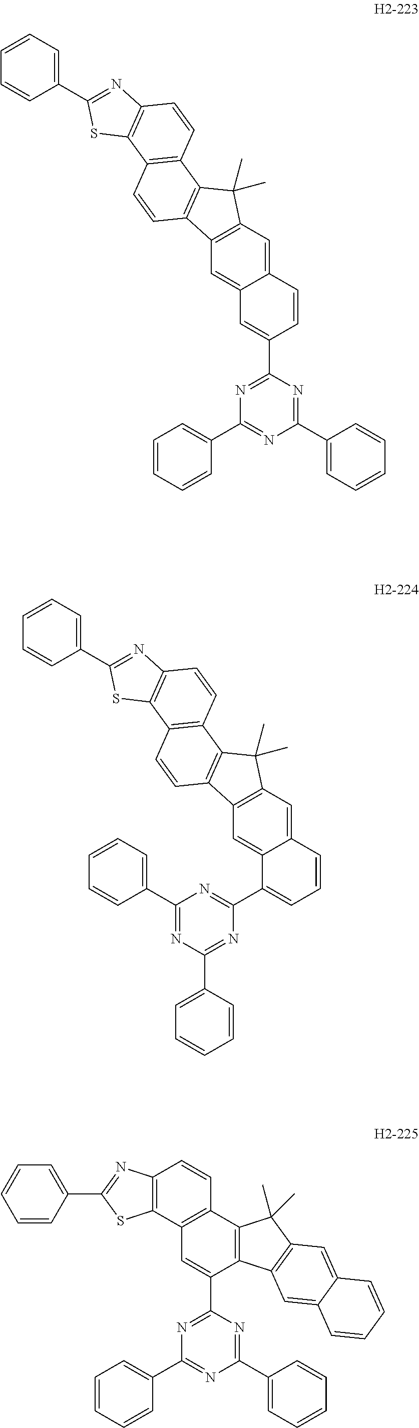

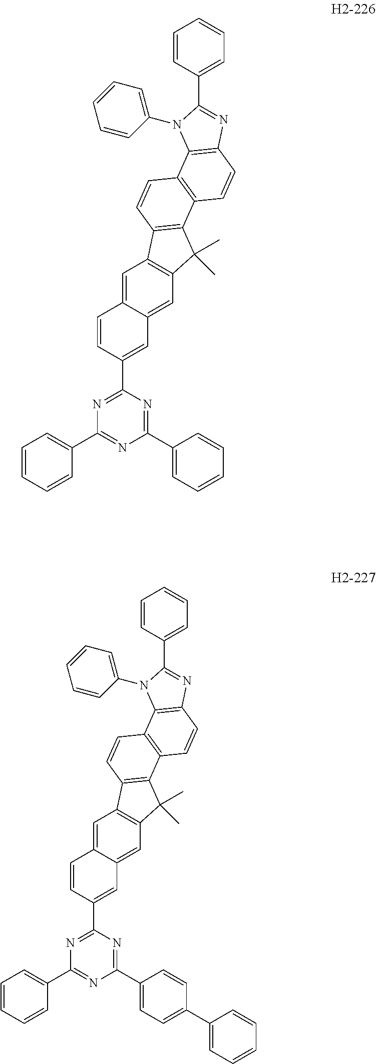 [SMILES: CC1(C)c2cc3cc(-c4nc(-c5ccccc5)nc(-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)n(-c5ccccc5)c43)c21]

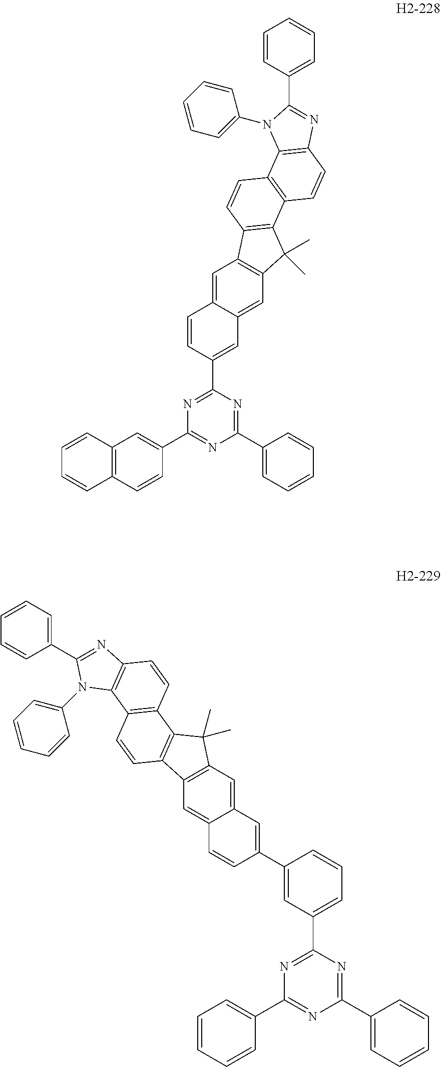 [SMILES: CC1(C)c2cc3cc(-c4nc(-c5ccccc5)nc(-c5ccc6ccccc6c5)n4)ccc3cc2-c2ccc3c(ccc4nc(-c5ccccc5)n(-c5ccccc5)c43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)n(-c5ccccc5)c43)c21]

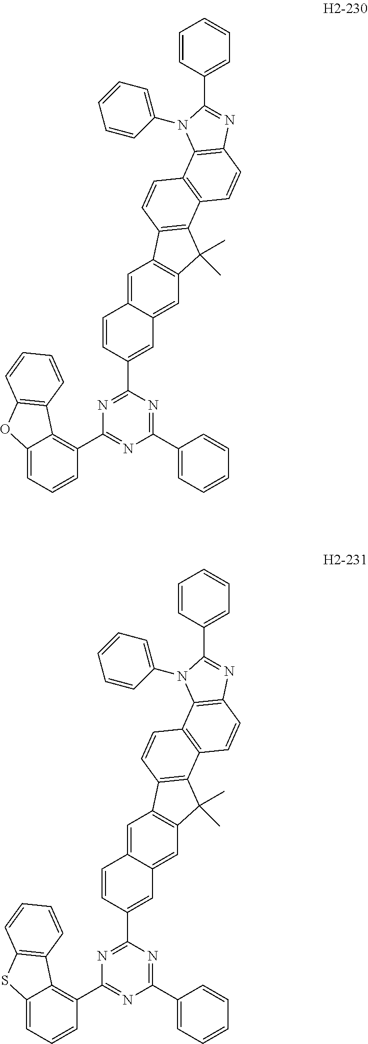 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)n(-c5ccccc5)c43)c21]

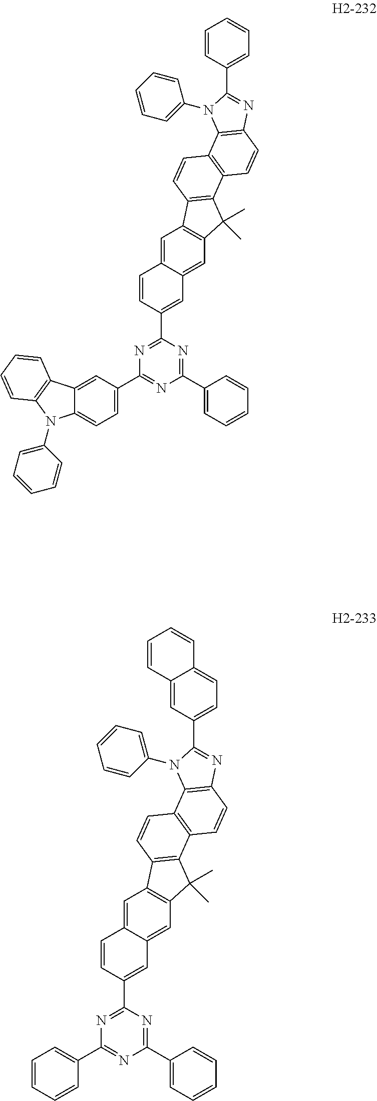 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccccc5)n4)ccc3cc2-c2ccc3c(ccc4nc(-c5ccc6ccccc6c5)n(-c5ccccc5)c43)c21]

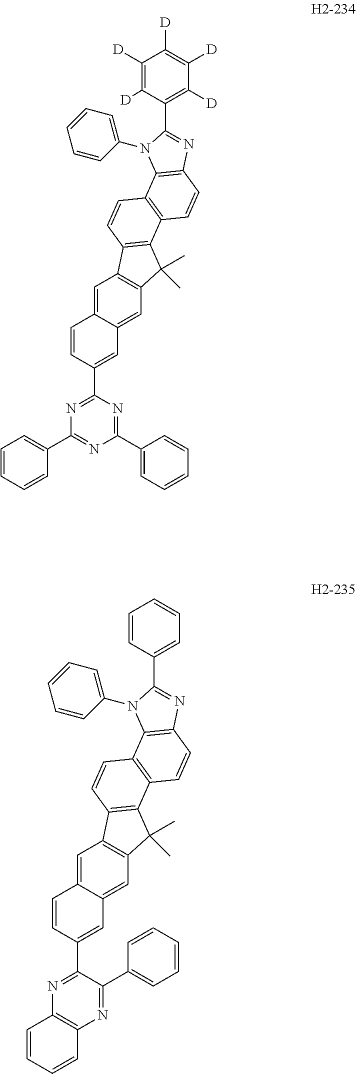 [SMILES: [2H]c1c([2H])c([2H])c(-c2nc3ccc4c5c(ccc4c3n2-c2ccccc2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)n(-c5ccccc5)c43)c21]

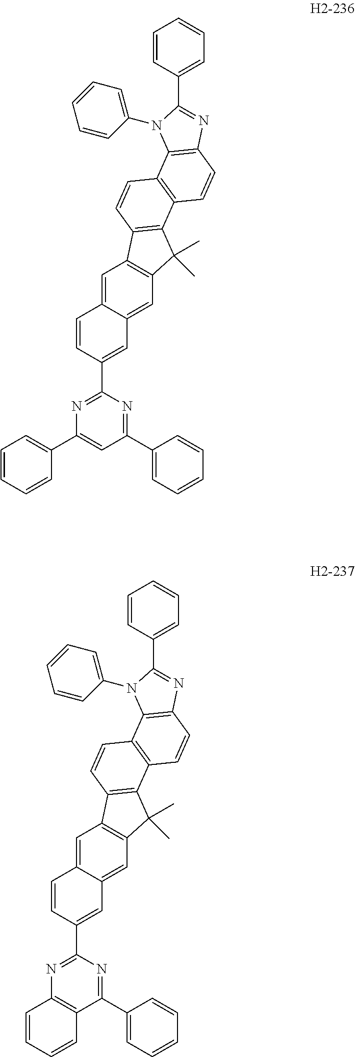 [SMILES: CC1(C)c2cc3cc(-c4nc(-c5ccccc5)cc(-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)c5ccccc5n4)ccc3cc2-c2ccc3c(ccc4nc(-c5ccccc5)n(-c5ccccc5)c43)c21]

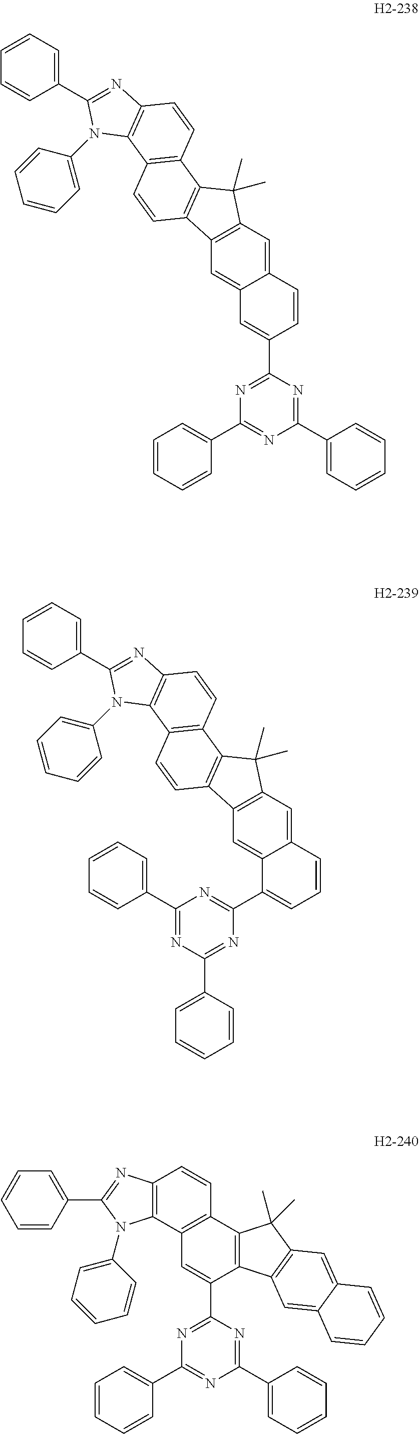 [SMILES: CC1(C)c2cc3ccc(-c4nc(-c5ccccc5)nc(-c5ccccc5)n4)cc3cc2-c2ccc3c(ccc4nc(-c5ccccc5)n(-c5ccccc5)c43)c21 ; CC1(C)c2cc3cccc(-c4nc(-c5ccccc5)nc(-c5ccccc5)n4)c3cc2-c2ccc3c(ccc4nc(-c5ccccc5)n(-c5ccccc5)c43)c21 ; CC1(C)c2cc3ccccc3cc2-c2c(-c3nc(-c4ccccc4)nc(-c4ccccc4)n3)cc3c(ccc4nc(-c5ccccc5)n(-c5ccccc5)c43)c21]

Hereinafter, an organic electroluminescent device will be described to which the aforementioned plurality of host materials and/or organic electroluminescent compound is (are) applied.

The organic electroluminescent device according to one embodiment includes a first electrode; a second electrode; and at least one organic layer(s) interposed between the first electrode and the second electrode. The organic layer may include a light-emitting layer, and the light-emitting layer may comprise a plurality of host materials comprising at least one first host compound represented by Formula 1 and at least one second host compound represented by Formula 2. Wherein, the weight ratio of the first host compound to the second host compound may be in the range of about 1:99 to about 99:1, preferably about 10:90 to about 90:10, more preferably about 30:70 to about 70:30, more preferably about 40:60 to about 60:40, even more preferably about 50:50 in the light-emitting layer.

According to one embodiment, the organic electroluminescent material of the present disclosure comprises at least one of compounds H1-1 to H1-193 which is(are) a first host compound, and at least one of compounds H2-1 to H2-240, which is(are) a second host compound. The plurality of host materials may be included in the same organic layer, for example, the same light-emitting layer, or may be included in different light-emitting layers.

According to another embodiment, the organic electroluminescent material of the present disclosure includes an organic electroluminescent compound represented by Formula 2′ alone or in a combination of two or more, and this organic electroluminescent material may be included in an organic layer of an organic electroluminescent device, for example, an electron transport layer, an electron buffer layer, a hole-blocking layer, or a light-emitting layer.

The organic layer may further comprise at least one layer selected from a hole injection layer, a hole transport layer, a hole auxiliary layer, a light-emitting auxiliary layer, an electron transport layer, an electron injection layer, an interlayer, a hole-blocking layer, an electron-blocking layer and an electron buffer layer, in addition to the light-emitting layer. The organic layer may further comprise an amine-based compound and/or an azine-based compound other than the light-emitting material according to the present disclosure. Specifically, the hole injection layer, the hole transport layer, the hole auxiliary layer, the light-emitting layer, the light-emitting auxiliary layer, or the electron-blocking layer may contain the amine-based compound, e.g., an arylamine-based compound and a styrylarylamine-based compound, etc., as a hole injection material, a hole transport material, a hole auxiliary material, a light-emitting material, a light-emitting auxiliary material, or an electron-blocking material. Further, the electron transport layer, the electron injection layer, the electron buffer layer, or the hole-blocking layer may contain the azine-based compound as an electron transport material, an electron injection material, an electron buffer material, or a hole-blocking material. Further, the organic layer may further comprise at least one metal selected from the group consisting of metals from Group 1, metals from Group 2, transition metals of the 4^{th} period, transition metals of the 5^{th} period, lanthanides, and organic metals of the d-transition elements of the Periodic Table, or at least one complex compound comprising such a metal.

The plurality of host materials according to one embodiment may be used as light-emitting materials for a white organic light-emitting device. The white organic light-emitting device has suggested various structures such as a parallel side-by-side arrangement method, a stacking arrangement method, CCM (color conversion material) method, etc., according to the arrangement of R (Red), G (Green), YG (yellowish green), or B (blue) light-emitting units. In addition, the plurality of host materials and/or the organic electroluminescent material according to one embodiment may also be applied to the organic electroluminescent device comprising a QD (quantum dot).

One of the first electrode and the second electrode may be an anode and the other may be a cathode. Wherein, the first electrode and the second electrode may each be formed as a transmissive conductive material, a transflective conductive material, or a reflective conductive material. The organic electroluminescent device may be a top emission type, a bottom emission type, or a dual-side emission type according to the kinds of the material forming the first electrode and the second electrode.

A hole injection layer, a hole transport layer, an electron-blocking layer, or a combination thereof can be used between the anode and the light-emitting layer. The hole injection layer may be multi-layered in order to lower the hole injection barrier (or hole injection voltage) from the anode to the hole transport layer or the electron-blocking layer, wherein each of the multi-layers may use two compounds simultaneously. Also, the hole injection layer may be doped as a p-dopant. Also, the electron-blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron-blocking layer may be multi-layered, and wherein each layer may use a plurality of compounds.

An electron buffer layer, a hole-blocking layer, an electron transport layer, an electron injection layer, or a combination thereof can be used between the light-emitting layer and the cathode. The electron buffer layer may be multi-layered in order to control the injection of the electron and improve the interfacial properties between the light-emitting layer and the electron injection layer, wherein each of the multi-layers may use two compounds simultaneously. The hole-blocking layer may be placed between the electron transport layer (or electron injection layer) and the light-emitting layer, and blocks the arrival of holes to the cathode, thereby improving the probability of recombination of electrons and holes in the light-emitting layer. The hole-blocking layer or the electron transport layer may also be multi-layered, wherein each layer may use a plurality of compounds. Further, the electron injection layer may be doped as an n-dopant.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. When an organic electroluminescent device includes two or more hole transport layers, the hole transport layer, which is further included, may be used as the hole auxiliary layer or the electron-blocking layer. The light-emitting auxiliary layer, the hole auxiliary layer, or the electron-blocking layer may have an effect of improving the efficiency and/or the lifespan of the organic electroluminescent device.

In the organic electroluminescent device of the present disclosure, preferably, at least one layer (hereinafter, “a surface layer”) selected from a chalcogenide layer, a halogenated metal layer, and a metal oxide layer may be placed on an inner surface(s) of one or both of a pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of an electroluminescent medium layer. The operation stability for the organic electroluminescent device may be obtained by the surface layer. Preferably, the chalcogenide includes SiOₓ(1≤x≤2), AlOₓ(1≤x≤1.5), SiON, SiAlON, etc.; the halogenated metal includes LiF, MgF₂, CaF₂, a rare earth metal fluoride, etc.; and the metal oxide includes Cs₂P, Li₂O, MgO, SrO, BaO, CaO, etc.

In addition, in the organic electroluminescent device of the present disclosure, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. Further, a reductive dopant layer may be employed as a charge generating layer to prepare an organic electroluminescent device having two or more light-emitting layers and emitting white light.

An organic electroluminescent device according to one embodiment may further comprise at least one dopant in the light-emitting layer.

The dopant comprised in the organic electroluminescent device of the present disclosure may be at least one phosphorescent or fluorescent dopant, preferably a phosphorescent dopant. The phosphorescent dopant material applied to the organic electroluminescent device of the present disclosure is not particularly limited, but may be preferably a metallated complex compound(s) of a metal atom(s) selected from iridium (Ir), osmium (Os), copper (Cu), and platinum (Pt), more preferably an ortho-metallated complex compound(s) of a metal atom(s) selected from iridium (Ir), osmium (Os), copper (Cu), and platinum (Pt), and even more preferably ortho-metallated iridium complex compound(s).

The dopant comprised in the organic electroluminescent device of the present disclosure may use the compound represented by the following Formula 101, but is not limited thereto.

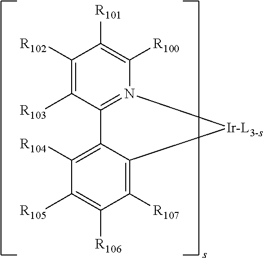

in Formula 101,
L is selected from any one of the following structures 1 to 3;

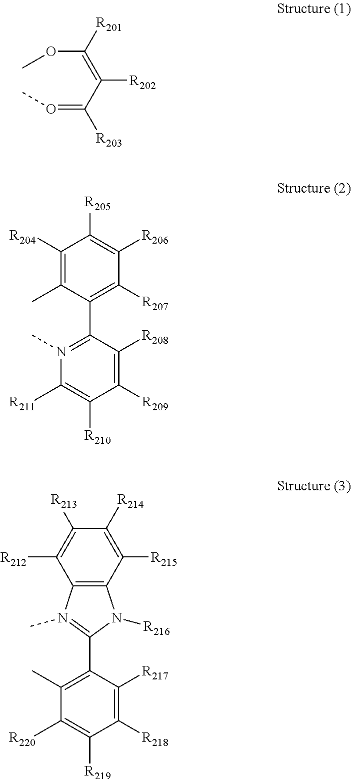

R₁₀₀ to R₁₀₃ each independently represent hydrogen, deuterium, halogen, (C₁-C₃₀)alkyl unsubstituted or substituted with deuterium and/or halogen, a substituted or unsubstituted (C₃-C₃₀)cycloalkyl, a substituted or unsubstituted (C₆-C₃₀)aryl, cyano, a substituted or unsubstituted (3- to 30-membered)heteroaryl, or a substituted or unsubstituted (C₁-C₃₀)alkoxy; or may be linked to the adjacent substituents to form a ring(s), for example to form a substituted or unsubstituted ring(s) with pyridine, such as, a substituted or unsubstituted quinoline, a substituted or unsubstituted benzopyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline;
R₁₀₄ to R₁₀₇ each independently represent hydrogen, deuterium, halogen, (C₁-C₃₀)alkyl unsubstituted or substituted with deuterium and/or halogen, a substituted or unsubstituted (C₃-C₃₀)cycloalkyl, a substituted or unsubstituted (C₆-C₃₀)aryl, a substituted or unsubstituted (3- to 30-membered)heteroaryl, cyano, or a substituted or unsubstituted (C₁-C₃₀)alkoxy; or may be linked to the adjacent substituents to form a substituted or unsubstituted ring(s), for example, to form a substituted or unsubstituted ring(s) with benzene, such as, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine;
R₂₀₁ to R₂₂₀ each independently represent hydrogen, deuterium, halogen, (C₁-C₃₀)alkyl unsubstituted or substituted with deuterium and/or halogen, a substituted or unsubstituted (C₃-C₃₀)cycloalkyl, or a substituted or unsubstituted (C₆-C₃₀)aryl; or may be linked to the adjacent substituents to form a substituted or unsubstituted ring(s); and
s represents an integer of 1 to 3.

Specifically, the specific examples of the dopant compound include the following, but are not limited thereto.

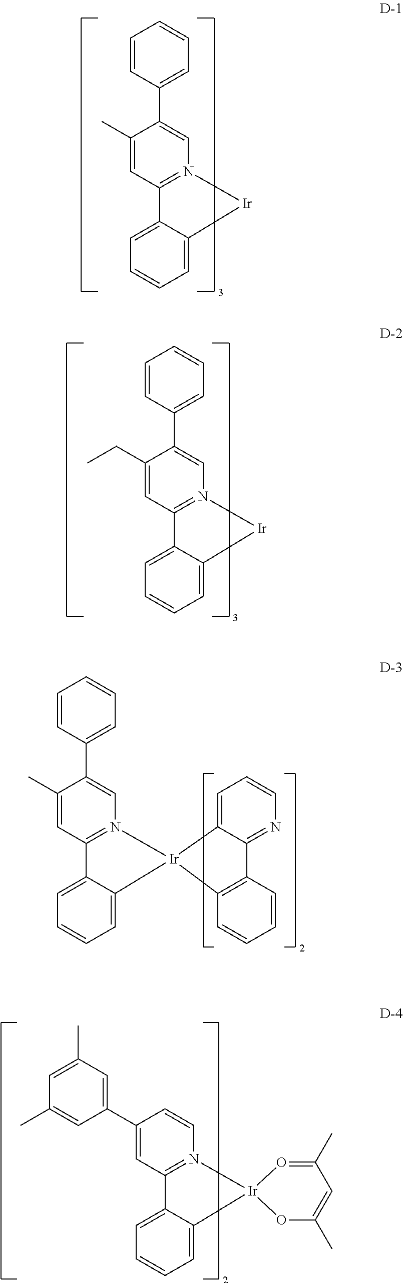

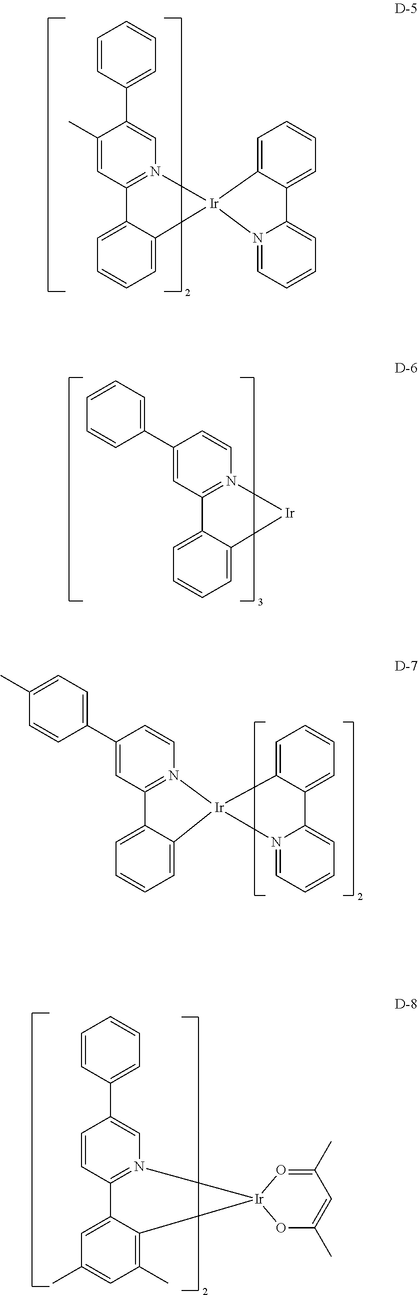

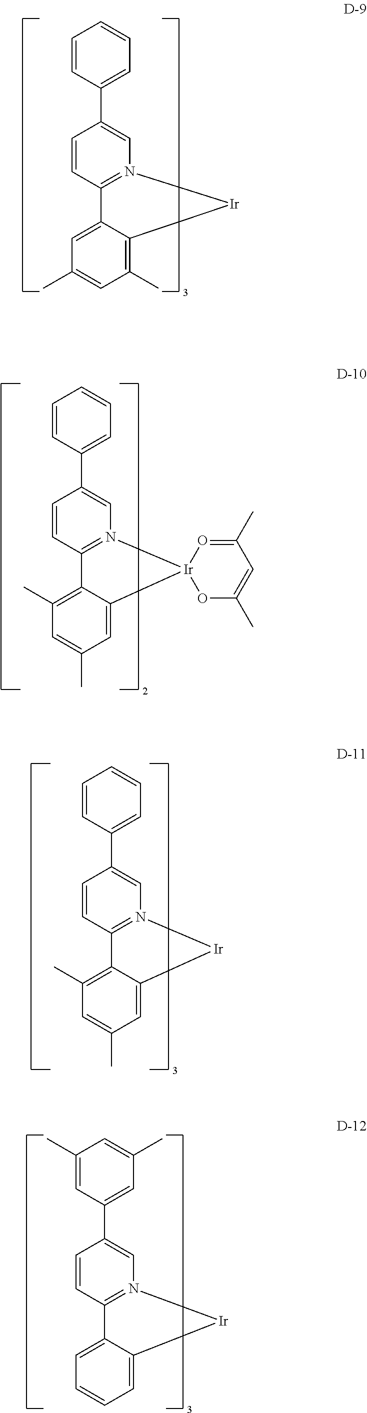

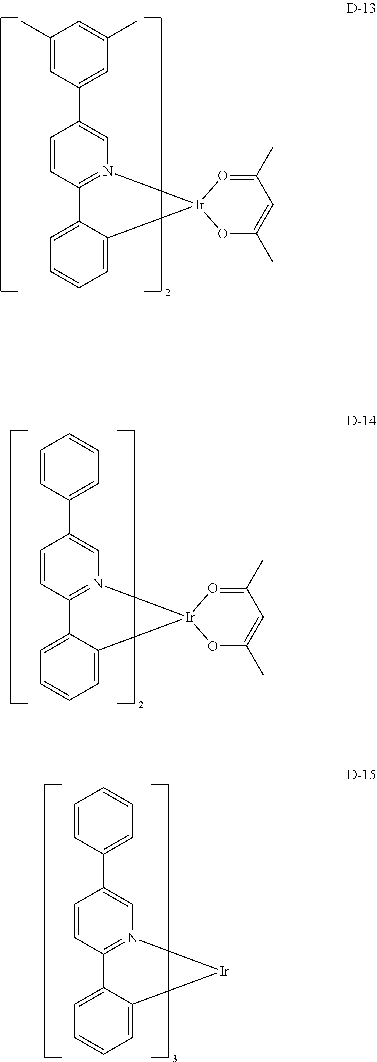

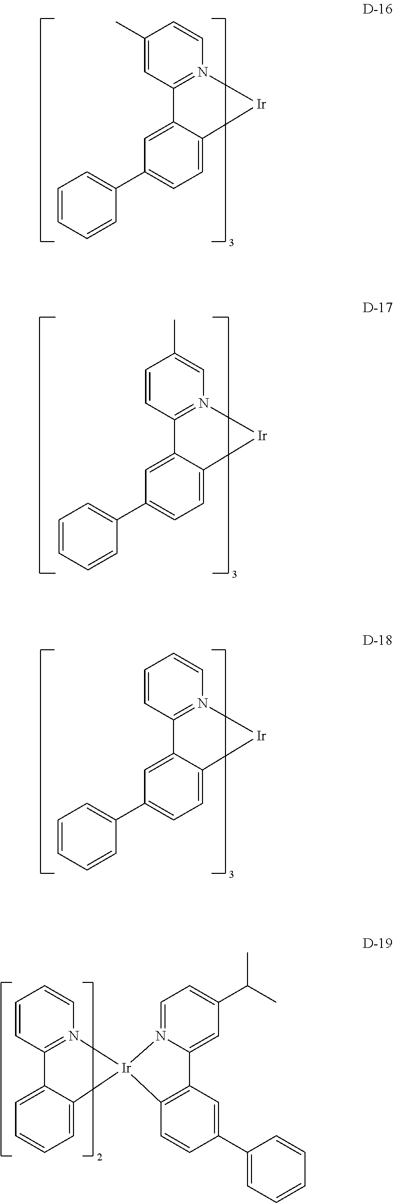

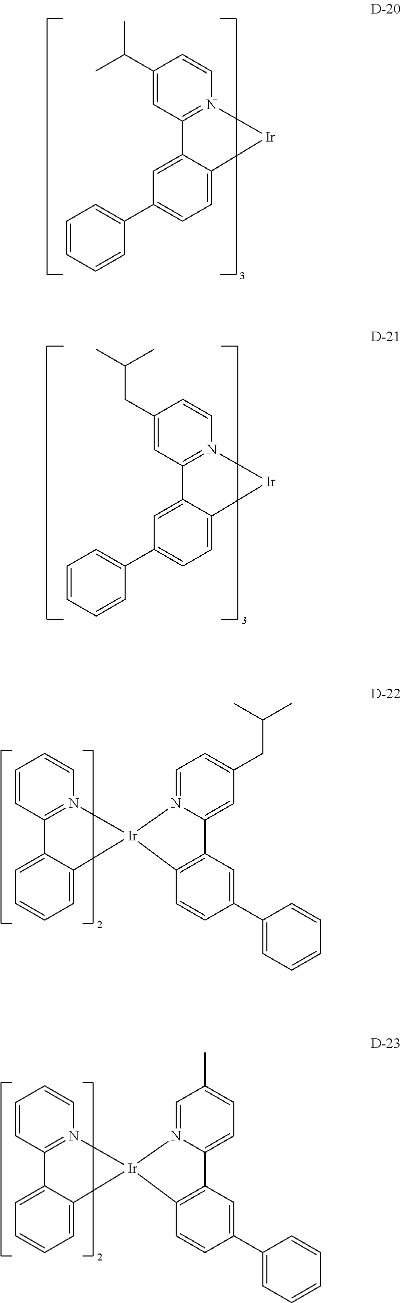

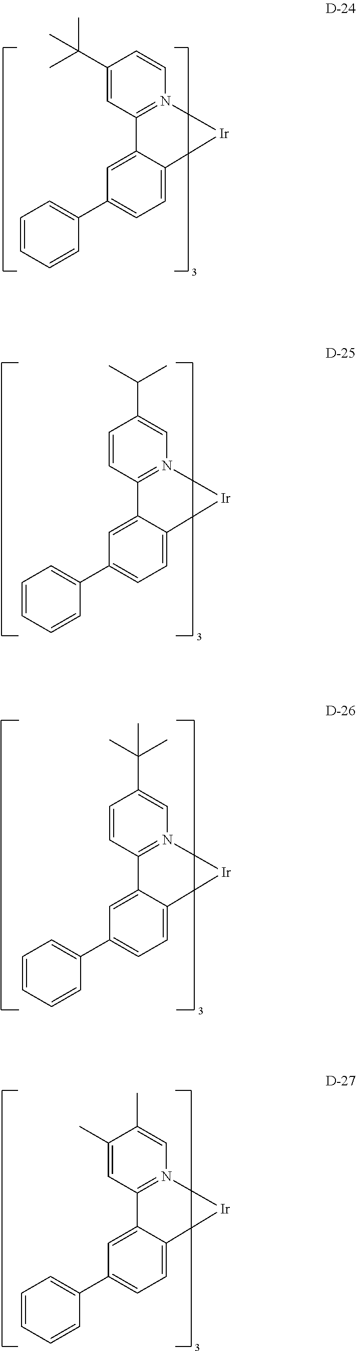

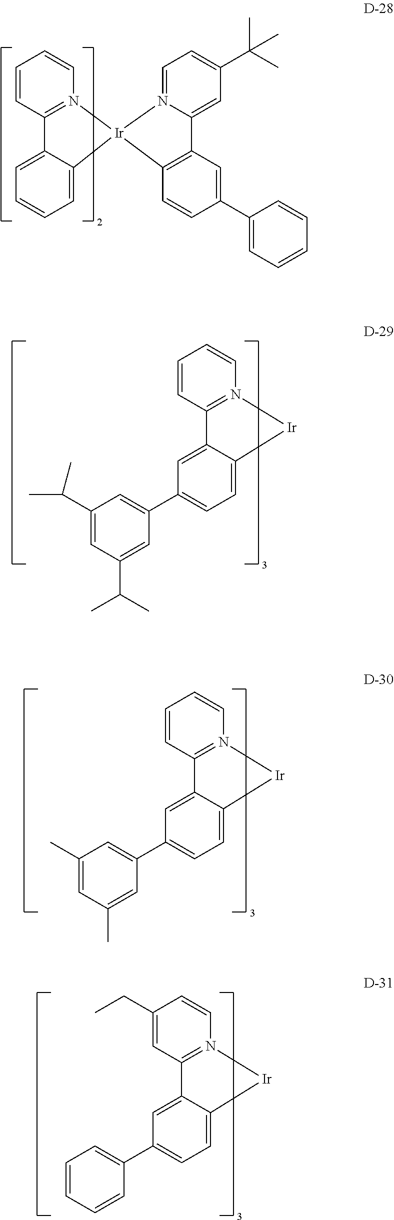

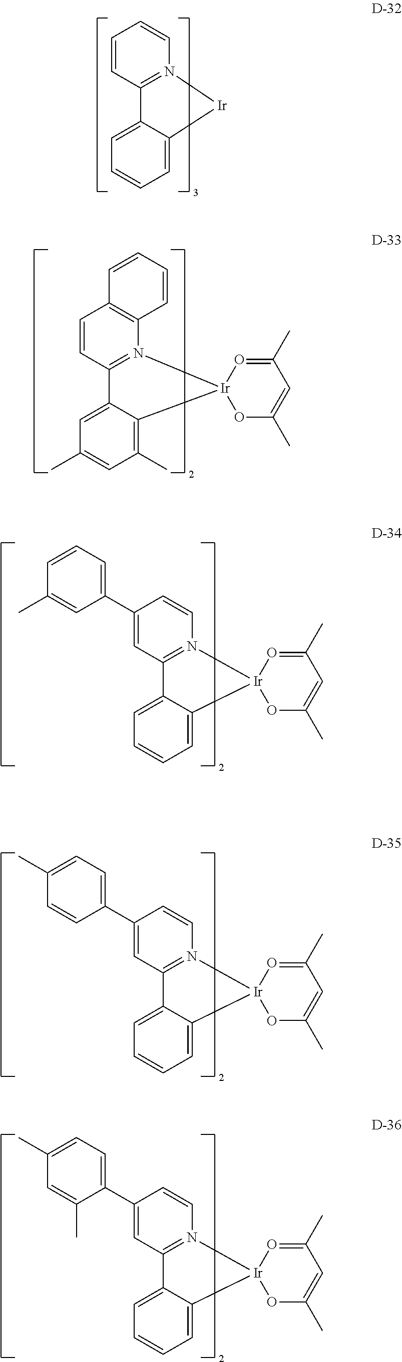

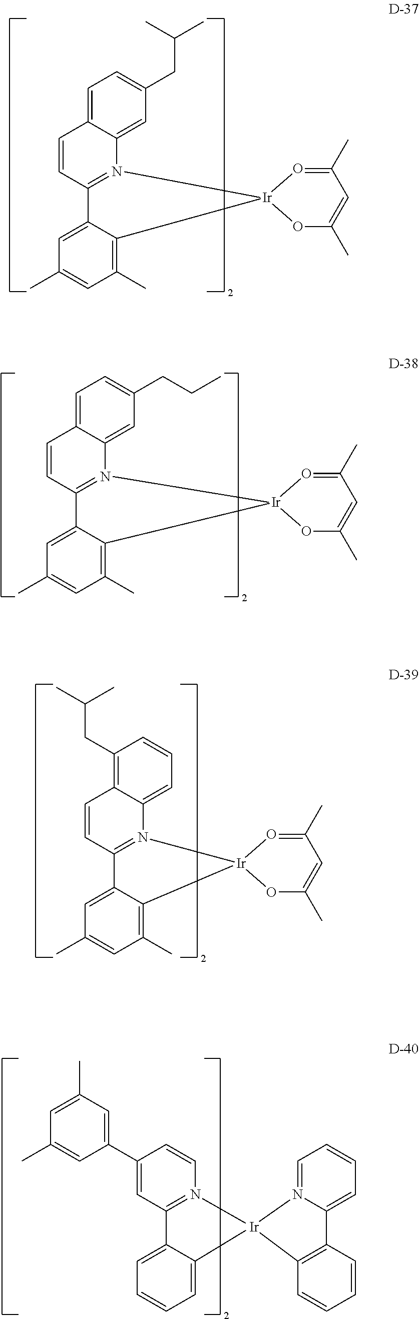

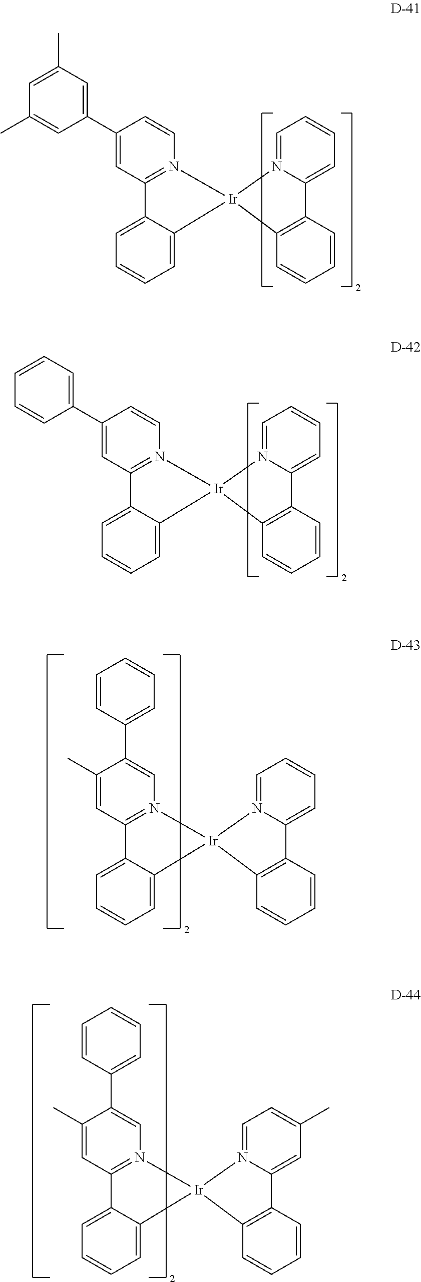

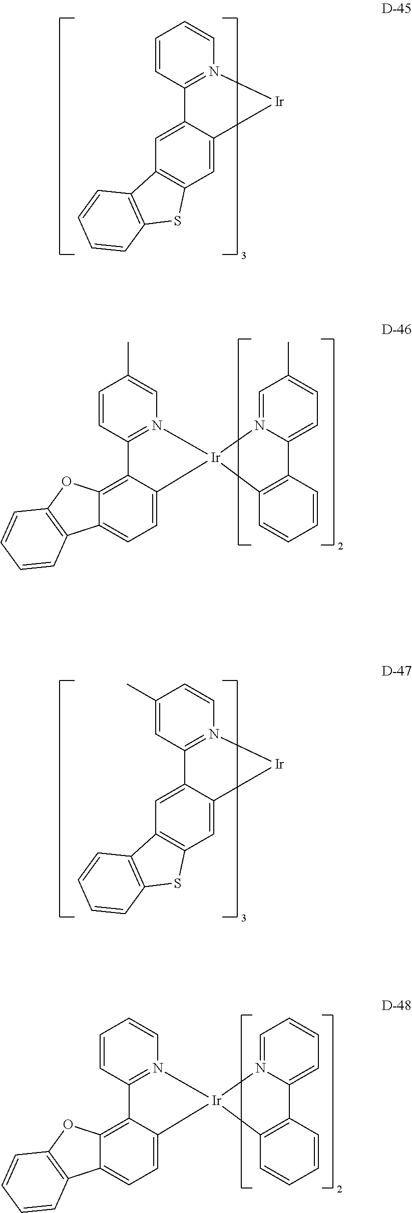

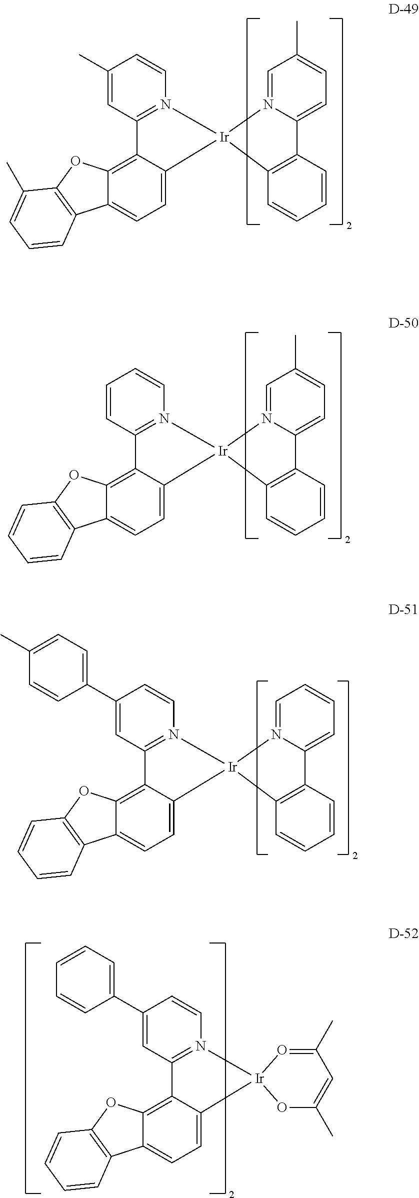

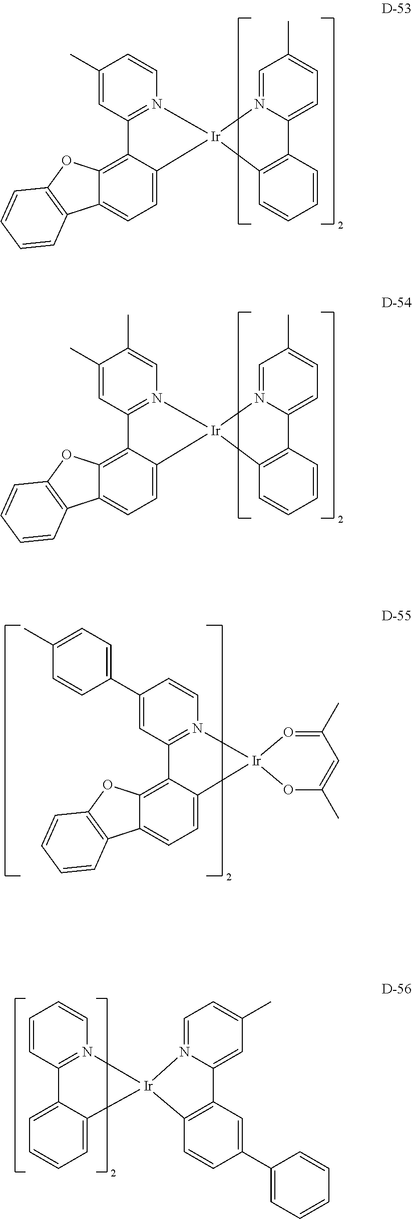

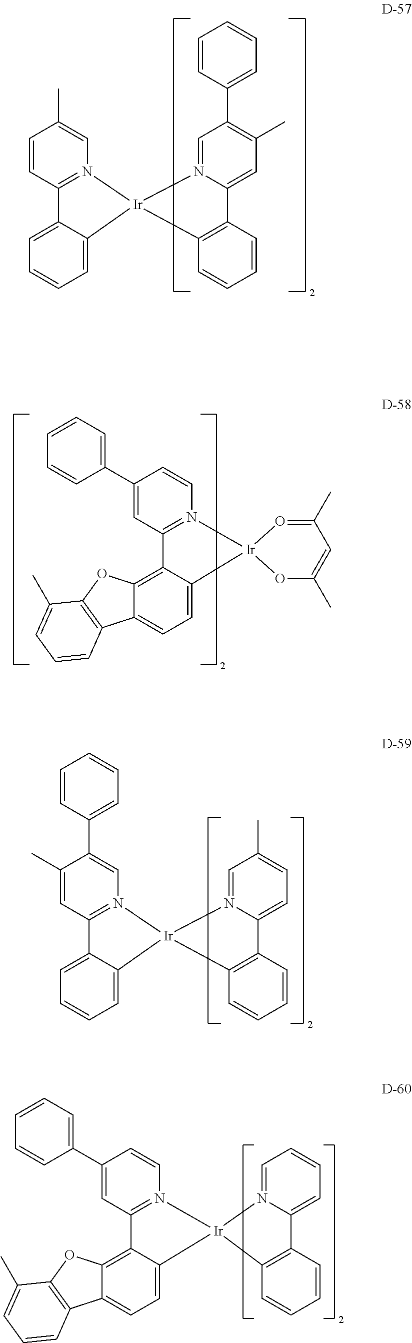

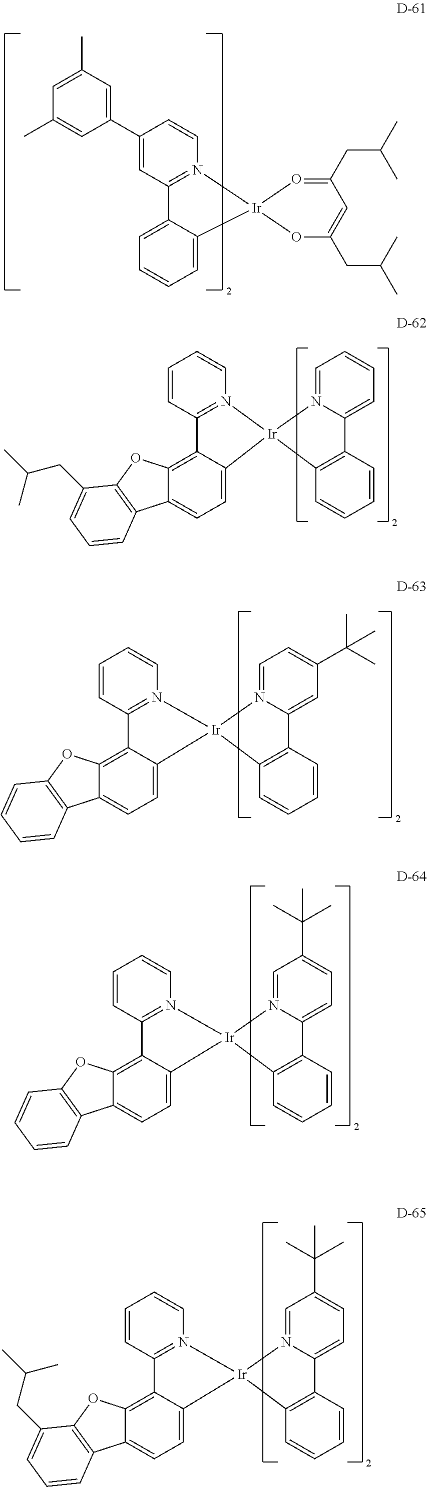

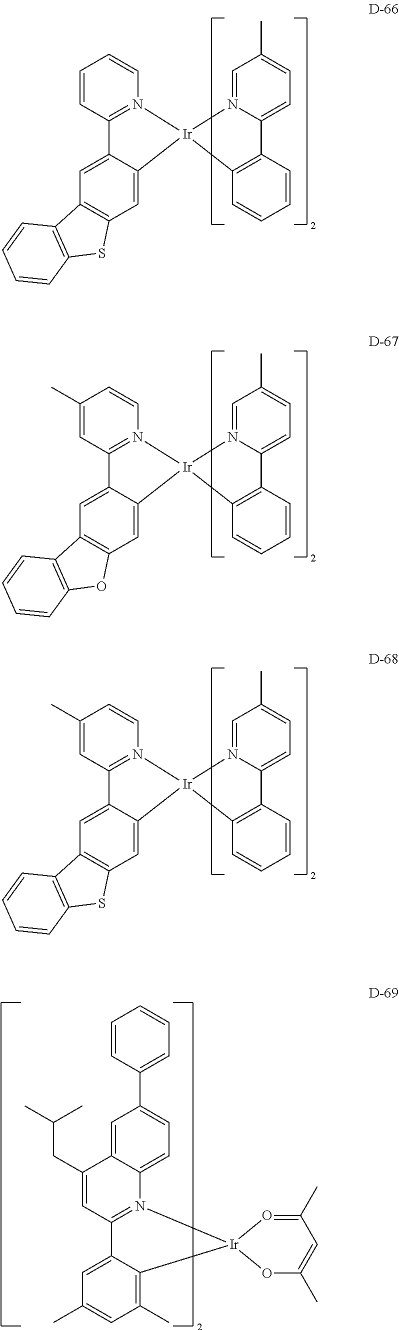

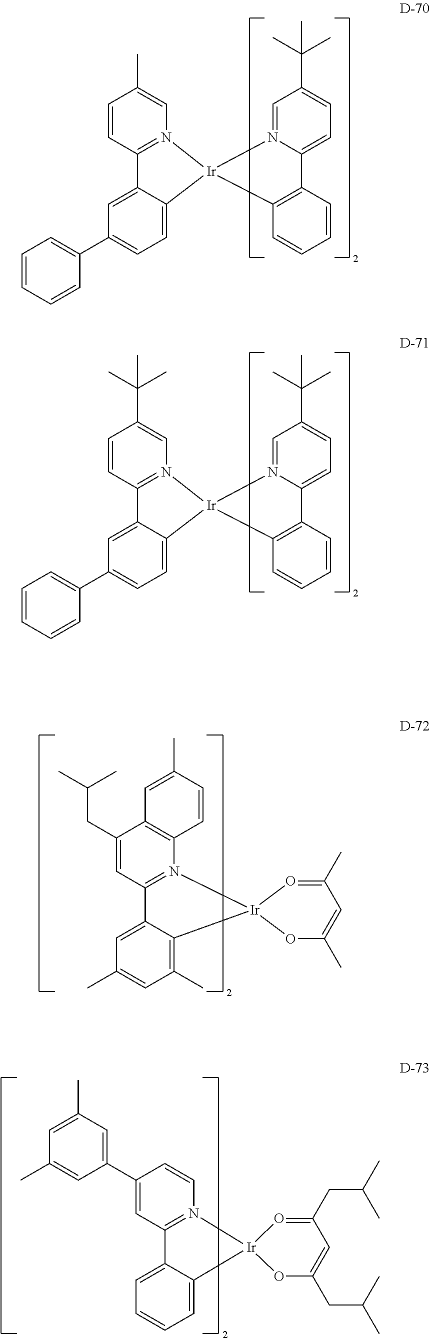

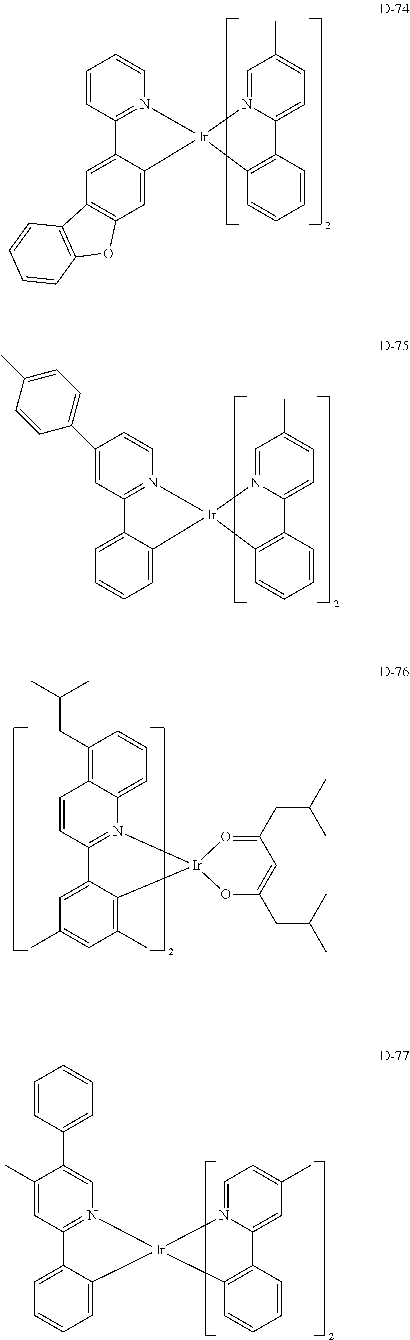

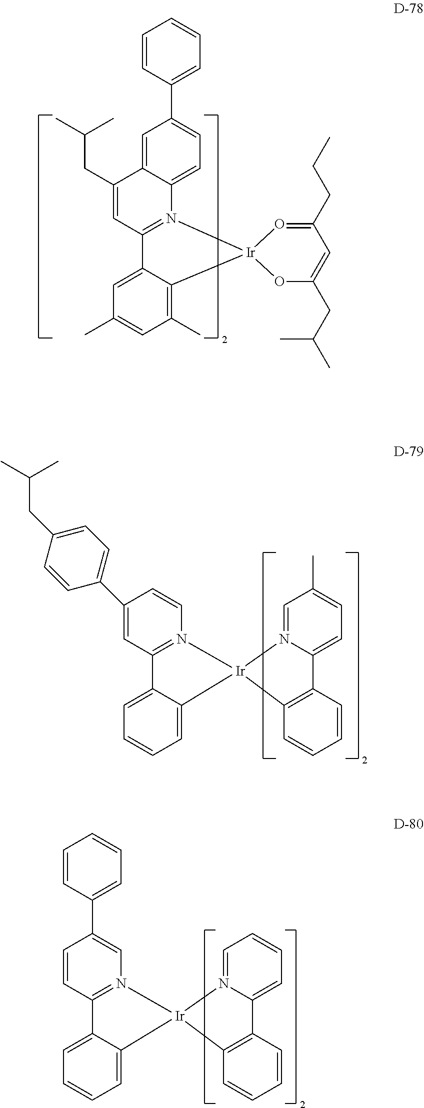

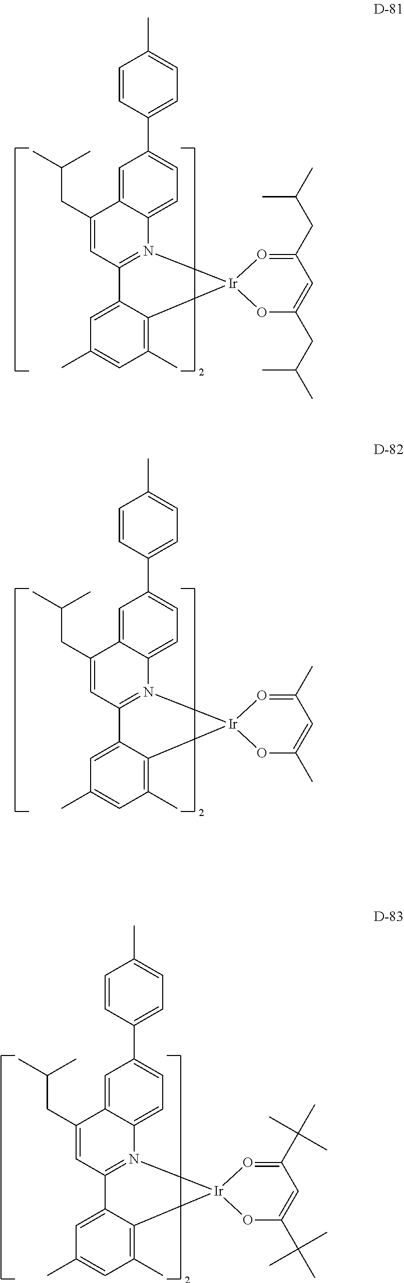

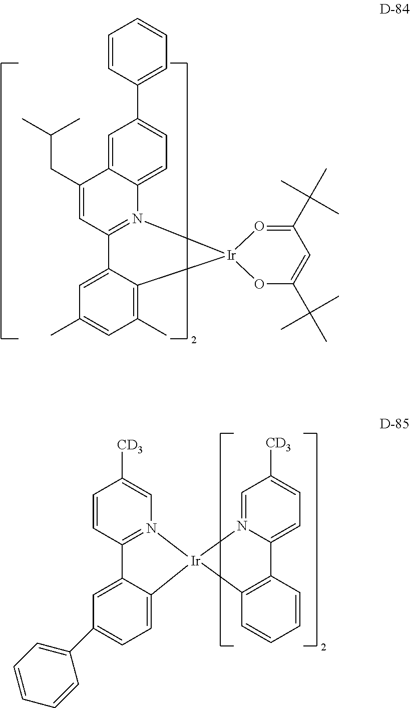

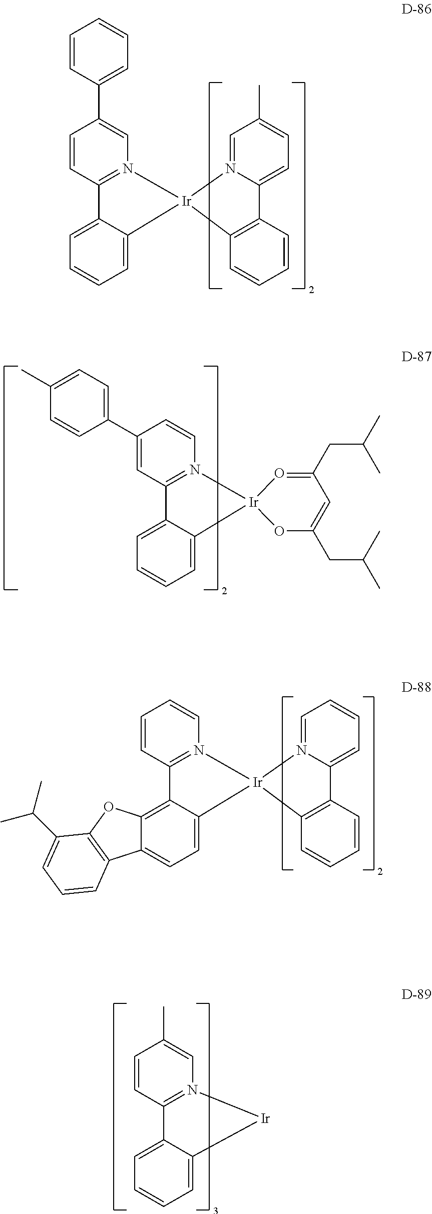

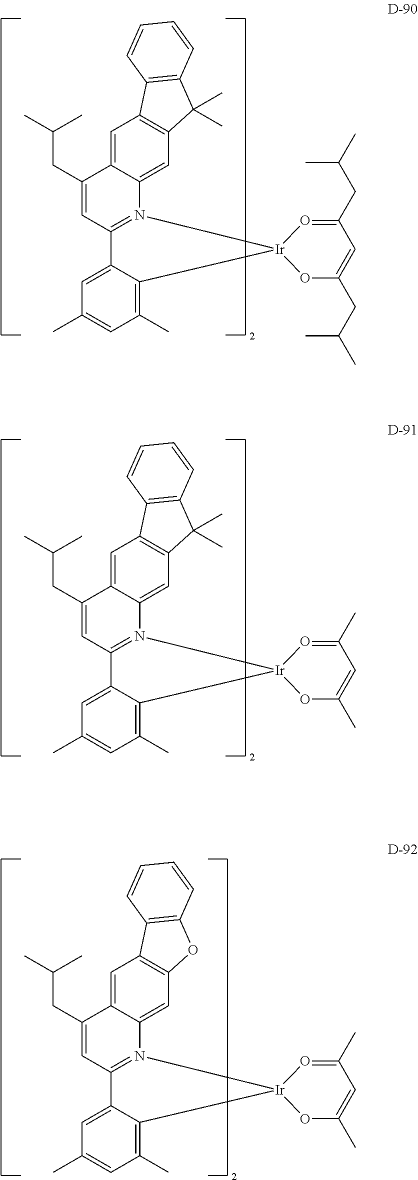

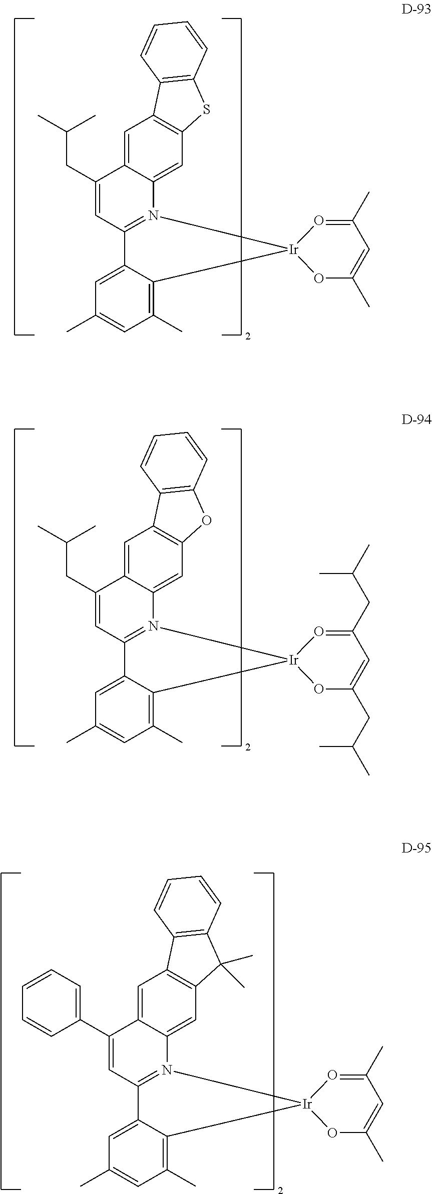

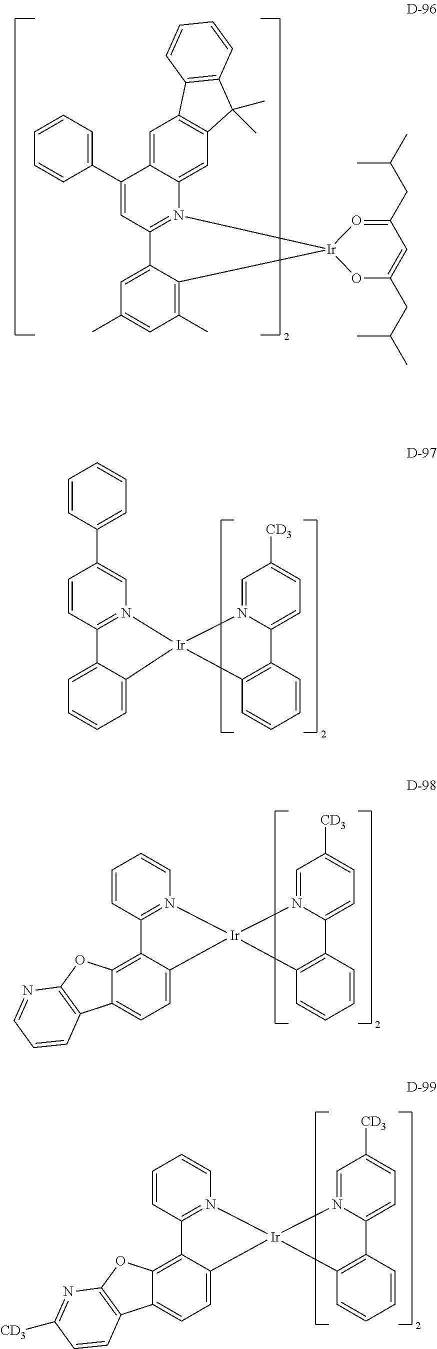

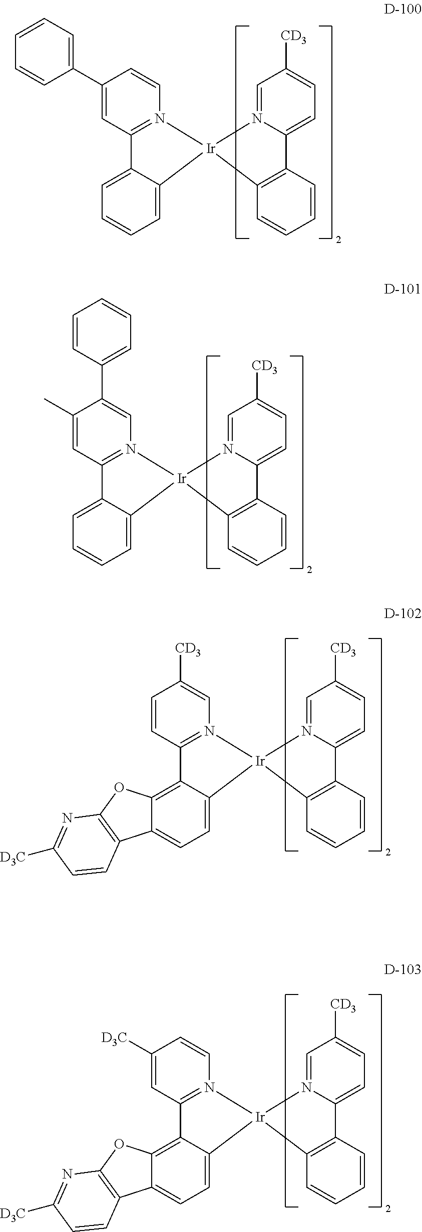

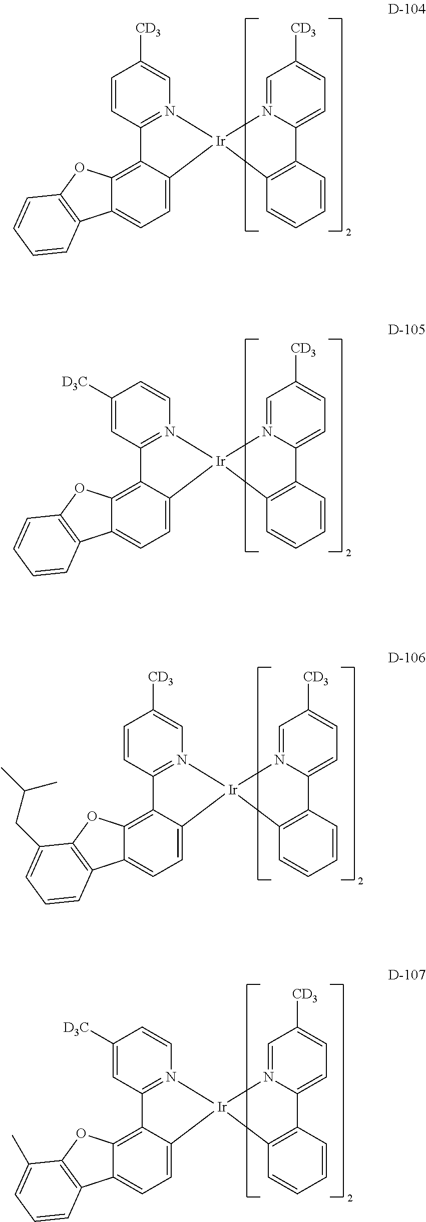

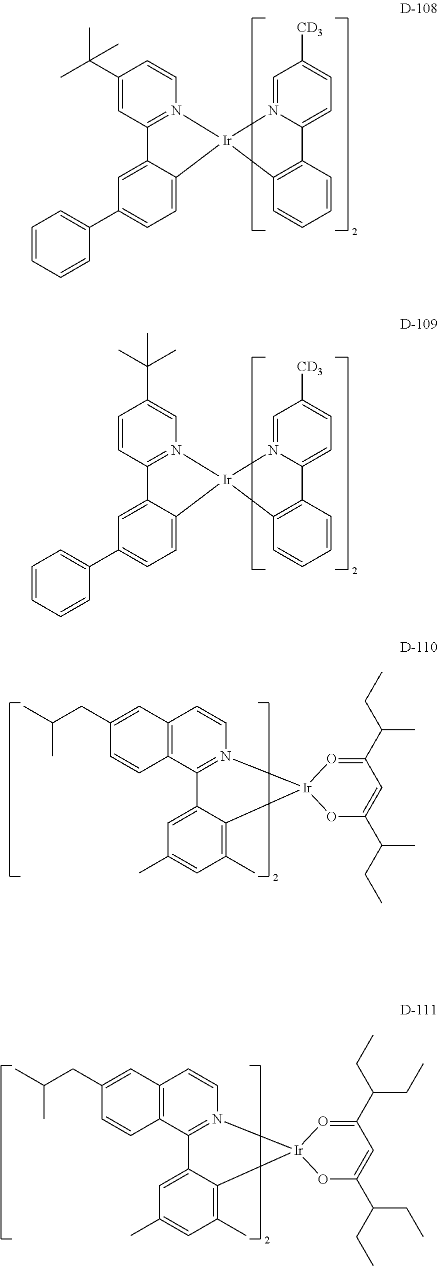

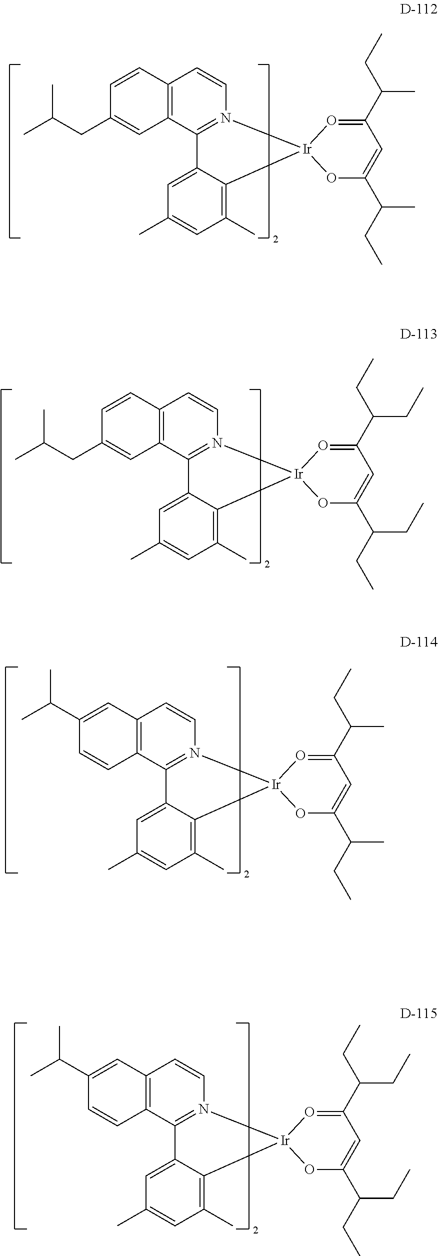

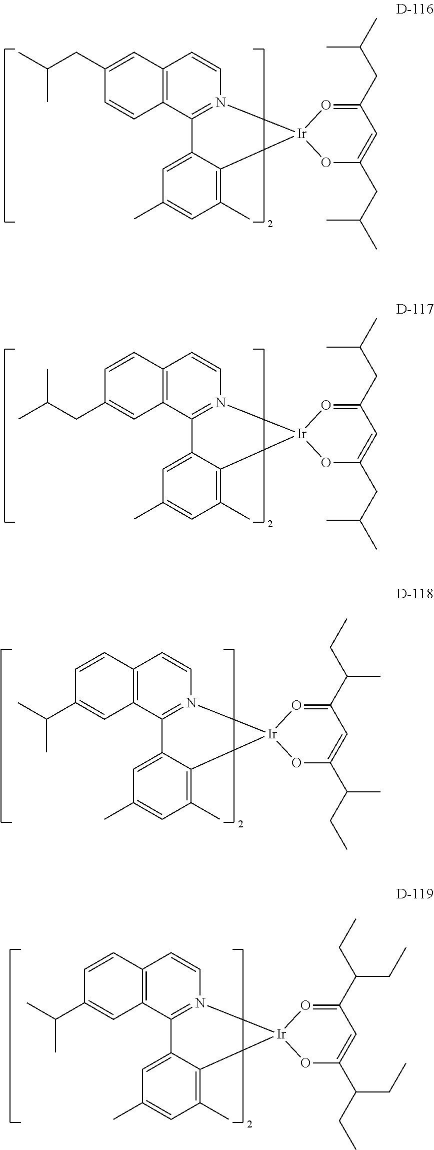

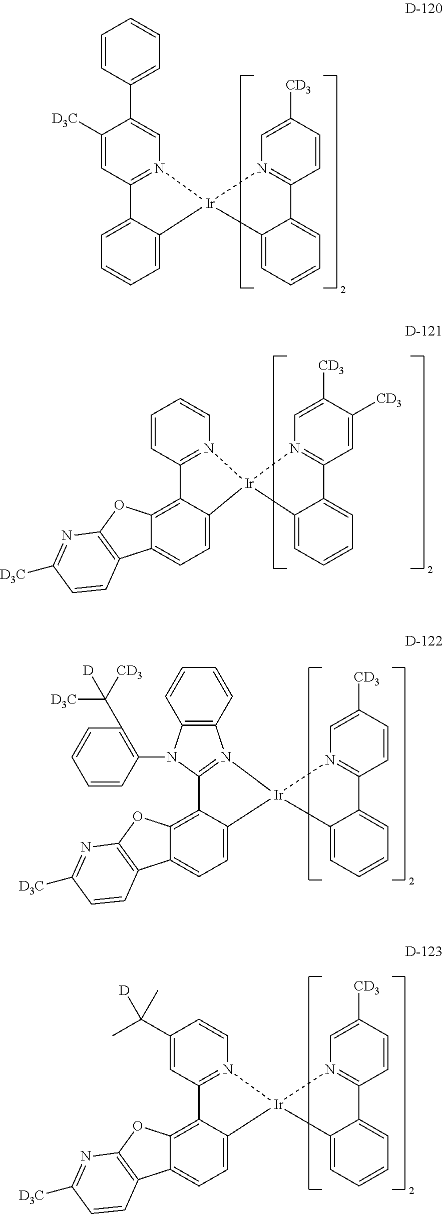

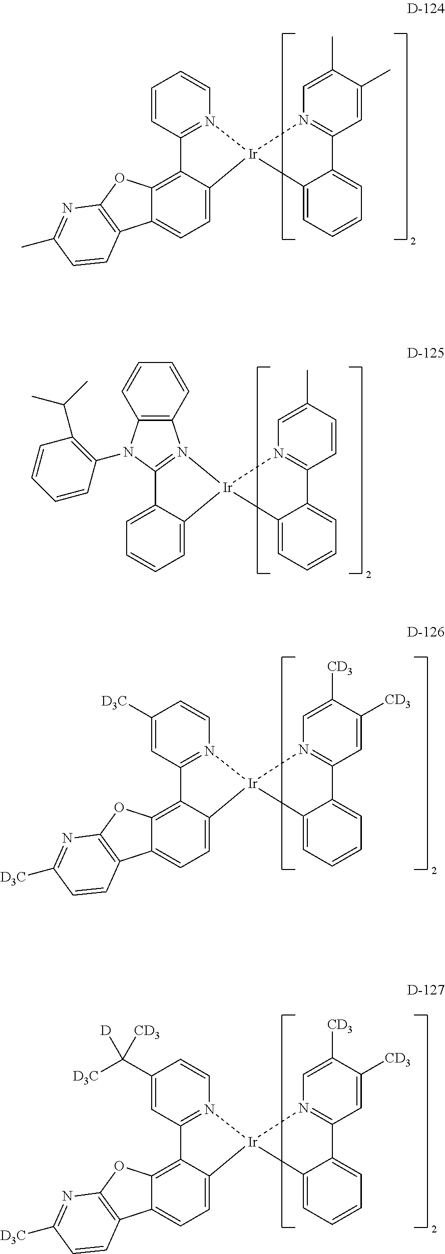

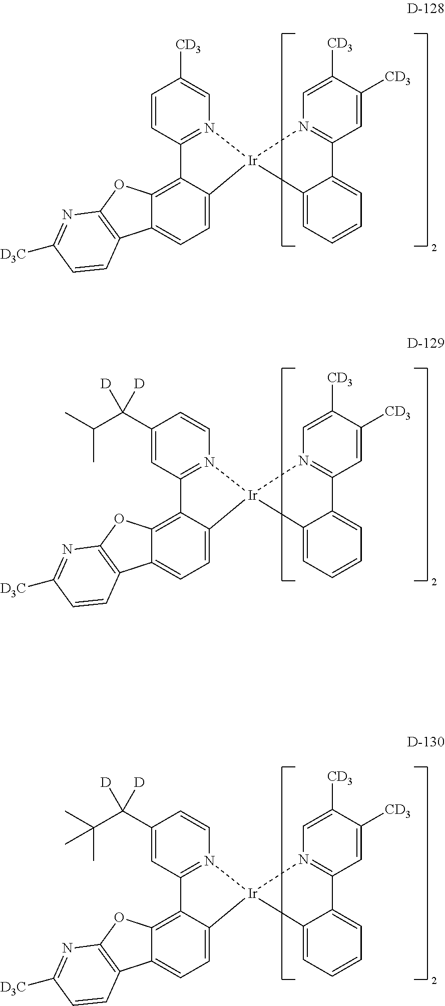

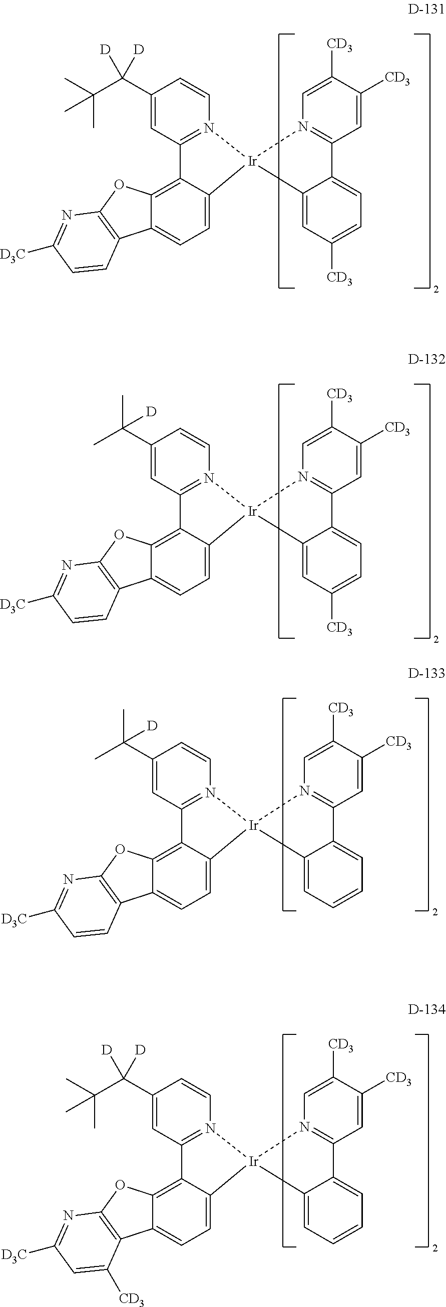

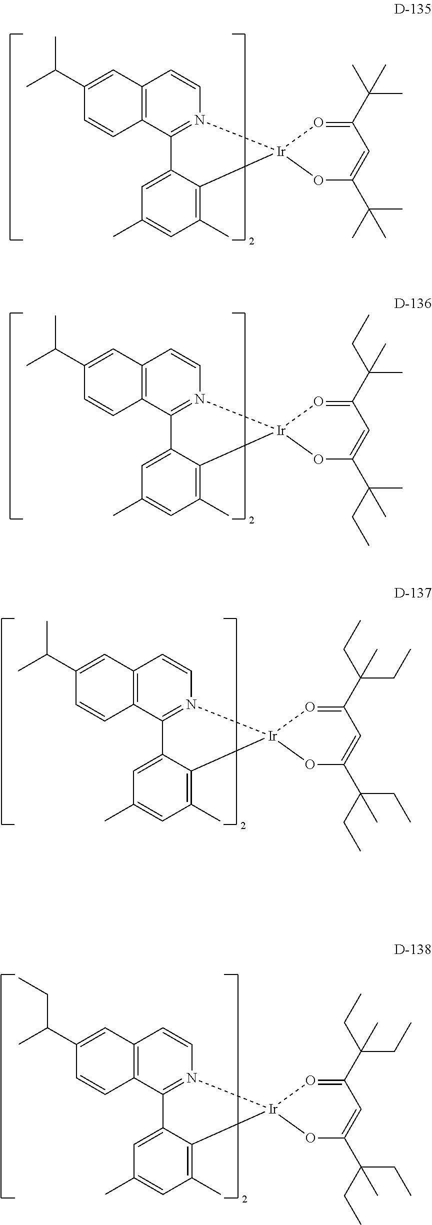

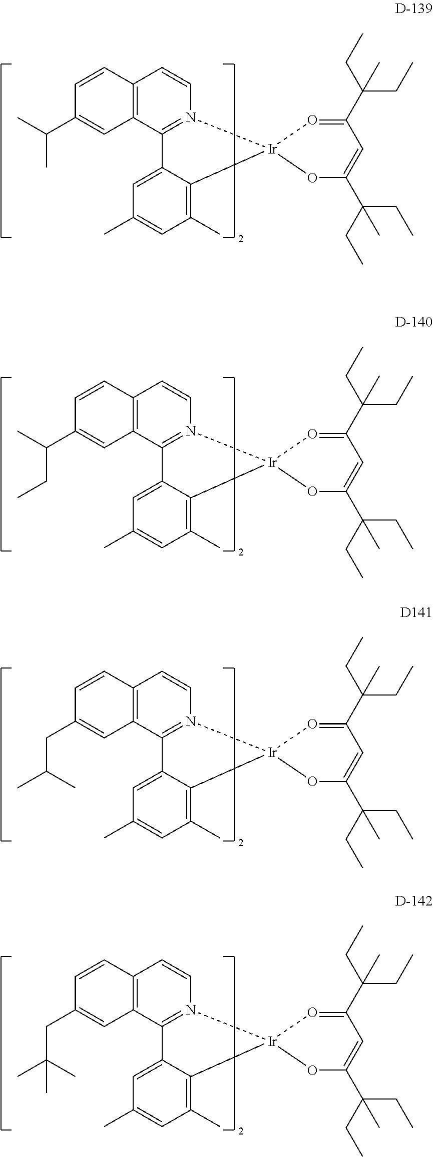

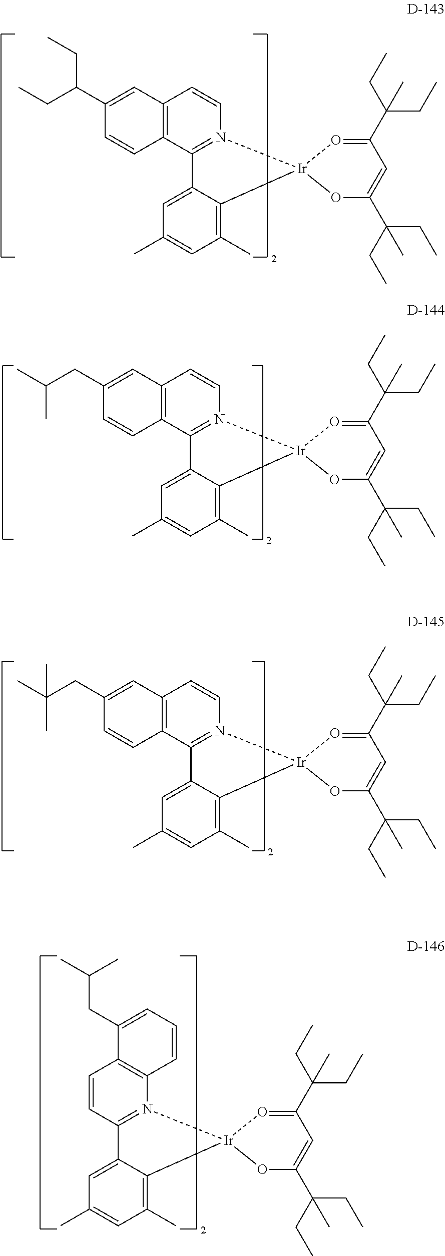

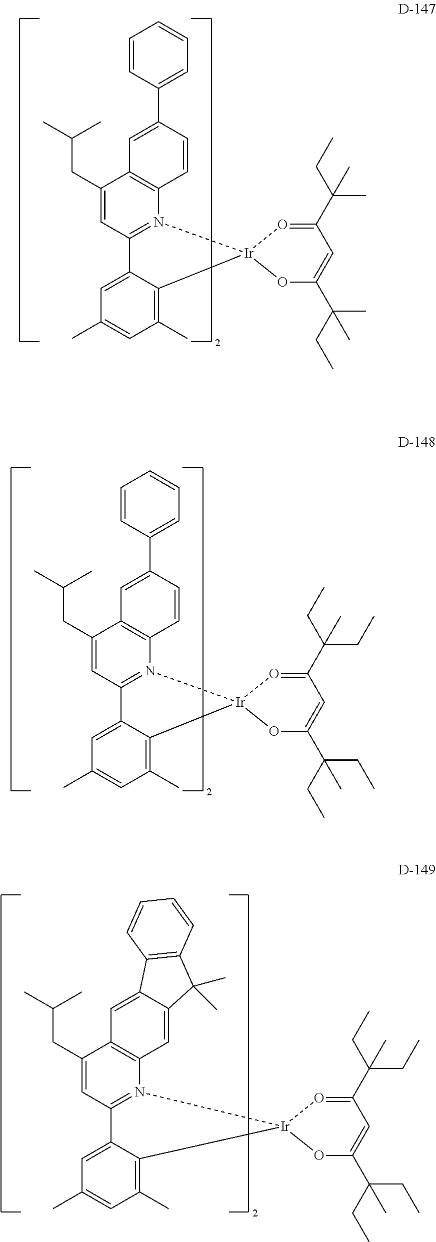

In order to form each layer of the organic electroluminescent device of the present disclosure, dry film-forming methods such as vacuum evaporation, sputtering, plasma, ion plating methods, etc. or wet film-forming methods such as spin coating, dip coating, flow coating methods, etc. can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film formation capability.

When forming a layer by the first host compound and the second host compound according to one embodiment, the layer can be formed by the above-listed methods, and can often be formed by co-deposition or mixed deposition. Co-deposition is a mixed deposition method in which two or more materials are put into respective individual crucible sources and a current is applied to both cells simultaneously to evaporate the materials and to perform mixed deposition; and the mixed deposition is a method in which two or more materials are mixed in one crucible source before deposition, and then a current is applied to one cell to evaporate the materials.

According to one embodiment, when the first host compound and the second host compound exist in the same layer or different layers in the organic electroluminescent device, the layers by the two host compounds may be separately formed. For example, after depositing the first host compound, a second host compound may be deposited.

According to one embodiment, the present disclosure can provide display devices comprising a plurality of host materials comprising a first host compound represented by Formula 1 and a second host compound represented by Formula 2. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of the host materials according to the present disclosure will be explained with reference to the synthesis method of a representative compound or intermediate compound in order to understand the present disclosure in detail.

### [EXAMPLE 1] PREPARATION OF COMPOUND H2-73

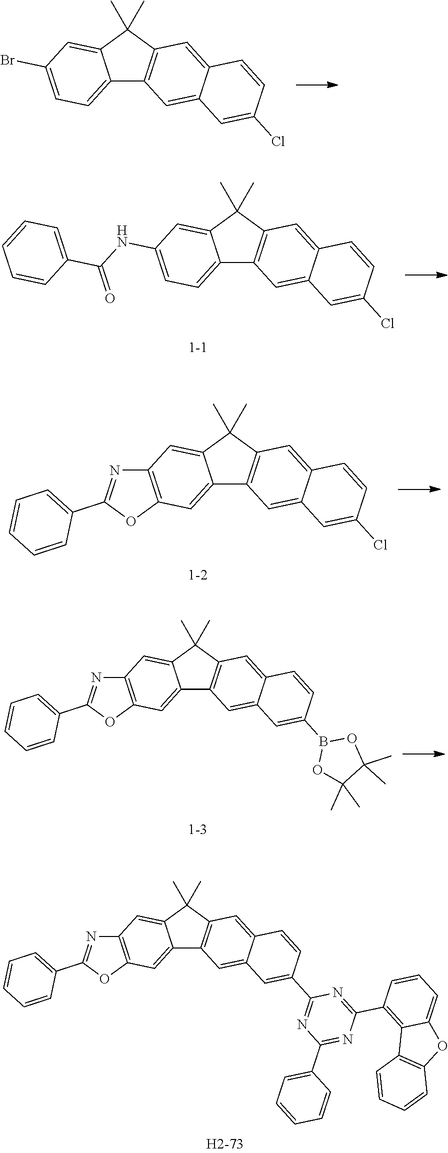 [SMILES: CC1(C)c2cc(Br)ccc2-c2cc3cc(Cl)ccc3cc21 ; CC1(C)c2cc(NC(=O)c3ccccc3)ccc2-c2cc3cc(Cl)ccc3cc21 ; CC1(C)c2cc3ccc(Cl)cc3cc2-c2cc3oc(-c4ccccc4)nc3cc21 ; CC1(C)c2cc3ccc(B4OC(C)(C)C(C)(C)O4)cc3cc2-c2cc3oc(-c4ccccc4)nc3cc21 ; CC1(C)c2cc3ccc(-c4nc(-c5ccccc5)nc(-c5cccc6oc7ccccc7c56)n4)cc3cc2-c2cc3oc(-c4ccccc4)nc3cc21]

### 1) Synthesis of Compound 1-1

2-Bromo-7-chloro-11,11-dimethyl-11H-benzo[b]fluorene (50 g, 140 mmol), benzamide (17 g, 140 mmol), Cul (40 g, 210 mmol), ethylenediamine (28.3 mL, 419 mmol), K₃PO₄ (59 g, 280 mmol), 350 mL of toluene, and 350 mL of xylene were added to a flask and dissolved, and then refluxed at 135° C. for 24 hours. After completion of the reaction, the organic layer was extracted with ethyl acetate, residual moisture was removed using magnesium sulfate, followed by drying, and column chromatography separation was then performed to obtain compound 1-1 (50 g, yield: 90%).

### 2) Synthesis of Compound 1-2

Compound 1-1 (10 g, 25 mmol) and Cu(OTf)₂ (13.6 g, 37 mmol) were added to a flask and dissolved in 312 mL of 1,2-dichlorobenzene (1,2-DCB), and then refluxed at 170° C. for 48 hours. After completion of the reaction, the organic layer was extracted with ethyl acetate, residual moisture was removed using magnesium sulfate, followed by drying, and column chromatography separation was then performed to obtain compound 1-2 (2.4 g, yield: 24%).

### 3) Synthesis of Compound 1-3

Compound 1-2 (3 g, 7.5 mmol), 4,4,4′,4′,5,5,5′,5′-octamethyl-2,2′-bi(1,3,2-dioxaborolane) (5.7 g, 22.7 mmol), Pd₂dba₃ (694 mg, 0.7 mmol), SPhos (622 mg, 1.5 mmol), and KOAc (2.2 g, 22.7 mmol) were added to a flask and dissolved in 50 mL of 1,4-dioxane, and then refluxed at 100° C. for 24 hours. After completion of the reaction, the organic layer was extracted with ethyl acetate, residual moisture was removed using magnesium sulfate, followed by drying, and column chromatography separation was then performed to obtain compound 1-3 (2.8 g, yield: 75%).

### 4) Synthesis of Compound H2-73

Compound 1-3 (2.7 g, 5.5 mmol), 2-chloro-4-(dibenzo[b,d]furan-1-yl)-6-phenyl-1,3,5-triazine (2.0 g, 5.5 mmol), Pd(PPh₃)₄(0.32 g, 0.27 mmol), K₂CO₃ (1.9 g, 14 mmol), 28 mL of toluene, 7 mL of ethanol, and 7 mL of H₂O were added to a flask and then stirred under reflux for 5 hours. After completion of the reaction, the organic layer was extracted with ethyl acetate, residual moisture was removed using magnesium sulfate, followed by drying, and column chromatography separation was then performed to obtain compound H2-73 (2.4 g, yield: 64%).

| | MW | M.P. |
|---|---|---|
| H2-73 | 682.78 | 333° C. |

### [EXAMPLE 2]PREPARATION OF COMPOUND H2-5

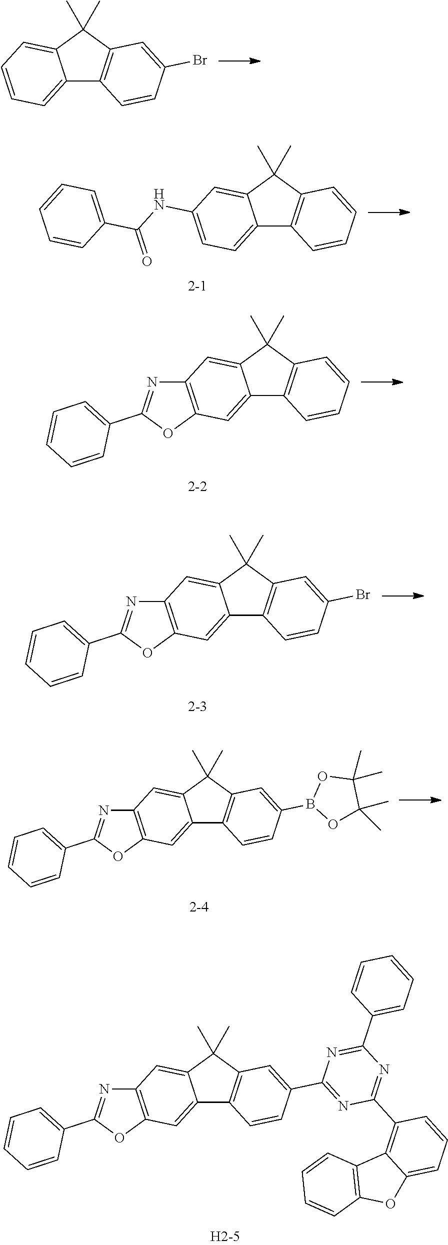 [SMILES: CC1(C)c2ccccc2-c2ccc(Br)cc21 ; CC1(C)c2ccccc2-c2ccc(NC(=O)c3ccccc3)cc21 ; CC1(C)c2ccccc2-c2cc3oc(-c4ccccc4)nc3cc21 ; CC1(C)c2cc(Br)ccc2-c2cc3oc(-c4ccccc4)nc3cc21 ; CC1(C)c2cc(B3OC(C)(C)C(C)(C)O3)ccc2-c2cc3oc(-c4ccccc4)nc3cc21 ; CC1(C)c2cc(-c3nc(-c4ccccc4)nc(-c4cccc5oc6ccccc6c45)n3)ccc2-c2cc3oc(-c4ccccc4)nc3cc21]

### 1) Synthesis of Compound 2-1

2-Bromo-9,9-dimethyl-9H-fluorene (30 g, 110 mmol), benzamide (13.3 g, 110 mmol), Cul (31.3 g, 165 mmol), ethylenediamine (22.2 mL, 330 mmol), K₃PO₄ (46.6 g, 220 mmol), and 550 mL of xylene were added to a flask and dissolved, and then refluxed at 150° C. for 24 hours. After completion of the reaction, the organic layer was extracted with ethyl acetate, residual moisture was removed using magnesium sulfate, followed by drying, and column chromatography separation was then performed to obtain compound 2-1 (26.4 g, yield: 77%).

### 2) Synthesis of Compound 2-2

Compound 2-1 (25 g, 80 mmol) and Cu(OTf)₂ (43.2 g, 120 mmol) were added to a flask and dissolved in 1,000 mL of 1,2-DCB, and then refluxed at 170° C. for 24 hours. After completion of the reaction, the organic layer was extracted with ethyl acetate, residual moisture was removed using magnesium sulfate, followed by drying, and column chromatography separation was then performed to obtain compound 2-2 (5.1 g, yield: 20%).

### 3) Synthesis of Compound 2-3

Compound 2-2 (5 g, 16 mmol) and NBS (5.7 g, 32 mmol) were dissolved in 160 mL of DMF, and then refluxed at 100° C. for 5 hours. After completion of the reaction, the organic layer was extracted with ethyl acetate, residual moisture was removed using magnesium sulfate, followed by drying, and column chromatography separation was then performed to obtain compound 2-3 (5.0 g, yield: 79%).

### 4) Synthesis of Compound 2-4

Compound 2-3 (2.2 g, 5.6 mmol), 4,4,4′,4′,5,5,5′,5′-octamethyl-2,2′-bi(1,3,2-dioxaborolane) (2.8 g, 11 mmol), PdCl₂(PPh₃)₂(395 mg, 0.5 mmol), and KOAc (1.1 g, 11 mmol) were dissolved in 37 mL of 1,4-dioxane, and then refluxed at 100° C. for 3 hours. After completion of the reaction, the organic layer was extracted with ethyl acetate, residual moisture was removed using magnesium sulfate, followed by drying, and column chromatography separation was then performed to obtain compound 2-4 (1.9 g, yield: 76%).

### 5) Synthesis of Compound H2-5

Compound 2-4 (1.8 g, 4.1 mmol), 2-chloro-4-(dibenzo[b,d]furan-1-yl)-6-phenyl-1,3,5-triazine (1.6 g, 4.5 mmol), Pd(PPh₃)₄(0.24 g, 0.2 mmol), K₂CO₃ (1.4 g, 10 mmol), 20 mL of toluene, 5 mL of ethanol, and 5 mL of H₂O were added to a flask and then stirred under reflux for 1.5 hours. After completion of the reaction, the organic layer was extracted with ethyl acetate, residual moisture was removed using magnesium sulfate, followed by drying, and column chromatography separation was then performed to obtain compound H2-5 (2.2 g, yield: 84%).

| | MW | M.P. |
|---|---|---|
| H2-5 | 632.72 | 269° C. |

### [EXAMPLE 3]PREPARATION OF COMPOUND H2-75

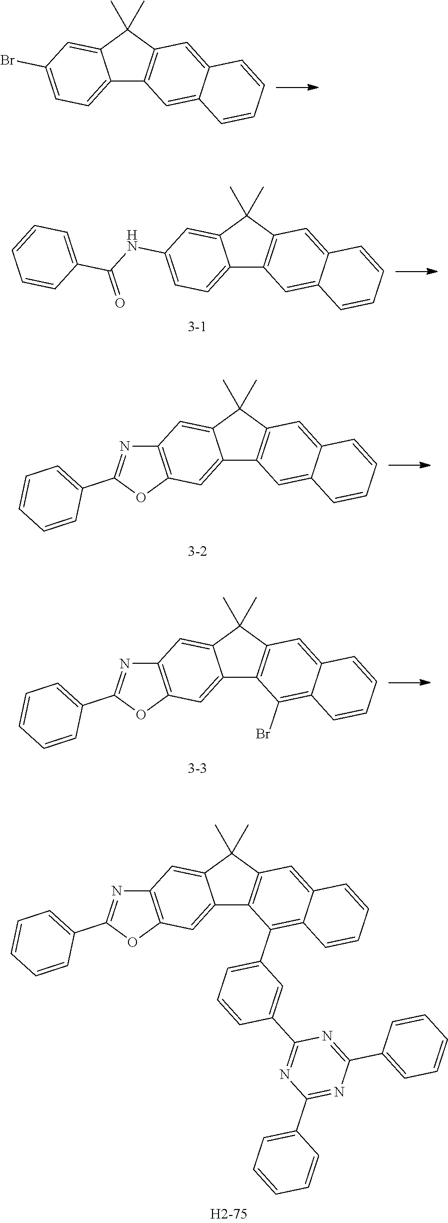 [SMILES: CC1(C)c2cc(Br)ccc2-c2cc3ccccc3cc21 ; CC1(C)c2cc(NC(=O)c3ccccc3)ccc2-c2cc3ccccc3cc21 ; CC1(C)c2cc3ccccc3cc2-c2cc3oc(-c4ccccc4)nc3cc21 ; CC1(C)c2cc3nc(-c4ccccc4)oc3cc2-c2c1cc1ccccc1c2Br ; CC1(C)c2cc3nc(-c4ccccc4)oc3cc2-c2c1cc1ccccc1c2-c1cccc(-c2nc(-c3ccccc3)nc(-c3ccccc3)n2)c1]

1) Synthesis of Compound 3-1

2-Bromo-11,11-dimethyl-11H-benzo[b]fluorene (50 g, 154 mmol), benzamide (18.7 g, 154 mmol), Cul (44 g, 232 mmol), ethylenediamine (31 mL, 464 mmol), K₃PO₄ (65.6 g, 309 mmol), and 770 mL of xylene were added to a flask and dissolved, and then refluxed at 150° C. for 24 hours. After completion of the reaction, the organic layer was extracted with ethyl acetate, residual moisture was removed using magnesium sulfate, followed by drying, and column chromatography separation was then performed to obtain compound 3-1 (53 g, yield: 94%).

### 2) Synthesis of Compound 3-2

Compound 3-1 (30 g, 82 mmol) and Cu(OTf)₂ (44.7 g, 123 mmol) were dissolved in 1,000 mL of 1,2-DCB, and then refluxed at 170° C. for 48 hours. After completion of the reaction, the organic layer was extracted with ethyl acetate, residual moisture was removed using magnesium sulfate, followed by drying, and column chromatography separation was then performed to obtain compound 3-2 (7.5 g, yield: 25%).

### 3) Synthesis of Compound 3-3

Compound 3-2 (6.5 g, 18 mmol) and NBS (3.2 g, 18 mmol) were dissolved in 180 mL of DMF, and then refluxed at 50° C. for 6 hours. After completion of the reaction, the organic layer was extracted with ethyl acetate, residual moisture was removed using magnesium sulfate, followed by drying, and column chromatography separation was then performed to obtain compound 3-3 (6.5 g, yield: 71%).

### 4) Synthesis of Compound H2-75

Compound 3-3 (2.9 g, 6.6 mmol), 2,4-diphenyl-6-(3-(4,4,5,5-tetramethyl-1,3,2-dioxaborolan-2-yl)phenyl)-1,3,5-triazine (3.4 g, 7.9 mmol), Pd(PPh₃)₄(0.38 g, 0.33 mmol), K₂CO₃ (2.3 g, 16 mmol), 32 mL of toluene, 8 mL of ethanol, and 8 mL of H₂O were added to a flask and then stirred under reflux for 1.5 hours. After completion of the reaction, the organic layer was extracted with ethyl acetate, residual moisture was removed using magnesium sulfate, followed by drying, and column chromatography separation was then performed to obtain compound H2-75 (1.7 g, yield: 38%).

| | MW | M.P. |
|---|---|---|
| H2-75 | 668.8 | 317° C. |

### [EXAMPLE 4]PREPARATION OF COMPOUND H2-74

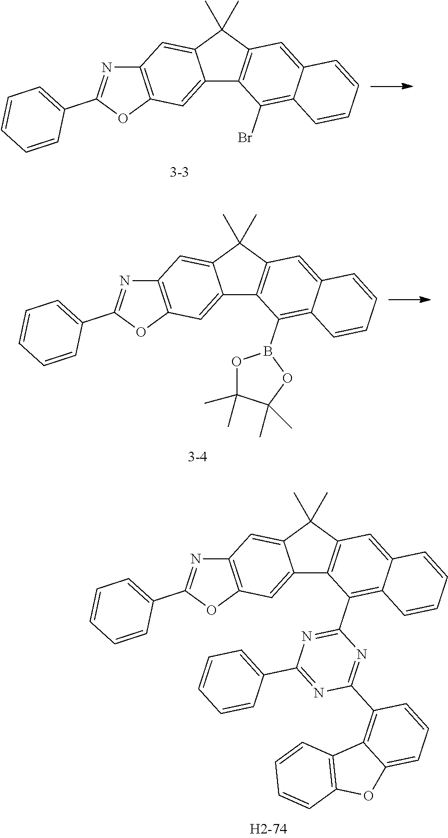 [SMILES: CC1(C)c2cc3nc(-c4ccccc4)oc3cc2-c2c1cc1ccccc1c2Br ; CC1(C)c2cc3nc(-c4ccccc4)oc3cc2-c2c1cc1ccccc1c2B1OC(C)(C)C(C)(C)O1 ; CC1(C)c2cc3nc(-c4ccccc4)oc3cc2-c2c1cc1ccccc1c2-c1nc(-c2ccccc2)nc(-c2cccc3oc4ccccc4c23)n1]

### 1) Synthesis of Compound 3-4

Compound 3-3 (6.5 g, 14.7 mmol), 4,4,4′,4′,5,5,5′,5′-octamethyl-2,2′-bi(1,3,2-dioxaborolane) (11.2 g, 44 mmol), PdCl₂(PPh₃)₂(1000 mg, 1.5 mmol), and KOAc (2.9 g, 30 mmol) were dissolved in 100 mL of 1,4-dioxane, and then refluxed at 100° C. for 3 hours. After completion of the reaction the organic layer was extracted with ethyl acetate, residual moisture was removed using magnesium sulfate, followed by drying, and column chromatography separation was then performed to obtain compound 3-4 (7 g, yield: 97%).

### 2) Synthesis of Compound H2-74

Compound 3-4 (4 g, 8.2 mmol), 2-chloro-4-(dibenzo[b,d]furan-1-yl)-6-phenyl-1,3,5-triazine (3.2 g, 9 mmol), Pd(PPh₃)₄(0.47 g, 0.41 mmol), K₂CO₃ (2.8 g, 20 mmol), 40 mL of toluene, 10 mL of ethanol, and 10 mL of H₂O were added to a flask and then stirred under reflux for 1.5 hours. After completion of the reaction, the organic layer was extracted with ethyl acetate, residual moisture was removed using magnesium sulfate, followed by drying, and column chromatography separation was then performed to obtain compound H2-74 (1.8 g, yield: 32%).

| | MW | M.P. |
|---|---|---|
| H2-74 | 682.78 | 274.2° C. |

### [Device Examples 1 to 4] Preparation of OLEDs by Co-Depositing the First Host Compound and the Second Host Compound According to the Present Disclosure

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter it was stored in isopropanol and used. Next, the ITO substrate was mounted on a substrate holder of a vacuum vapor deposition apparatus. Then, Compound HI-1 was introduced into a cell of the vacuum vapor deposition apparatus, while Compound HT-1 was introduced into another cell. The two materials were evaporated at different rates and Compound HI-1 was deposited in a doping amount of 3 wt % based on the total amount of Compound HI-1 and Compound HT-1 to form a hole injection layer having a thickness of 10 nm. Next, Compound HT-1 was deposited to form a first hole transport layer having a thickness of 80 nm on the hole injection layer. Next, compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host compound and the second host compound described in the following Table 1 were introduced into two cells of the vacuum vapor deposition apparatus as hosts, respectively, and Compound D-39 was introduced into another cell as a dopant. The two host materials were evaporated at a rate of 1:1 and the dopant material was evaporated at a different rate, simultaneously, and the dopant was deposited in a doping amount of 3 wt % based on the total amount of the hosts and dopant to form a light-emitting layer having a thickness of 40 nm on the second hole transport layer. Next, compounds ET-1 and EI-1 as electron transport materials were deposited at a weight ratio of 50:50 to form an electron transport layer having a thickness of 35 nm on the light-emitting layer. After depositing Compound EI-1 as an electron injection layer having a thickness of 2 nm on the electron transport layer, an AI cathode having a thickness of 80 nm was deposited on the electron injection layer by another vacuum vapor deposition apparatus. Thus, OLEDs were produced. Each compound used for all the materials were purified by vacuum sublimation under 10⁻⁶ Torr.

### [Device Comparative Example 1] Preparation of an OLED Comprising a Single Host

An OLED was manufactured in the same manner as in Device Example 1, except that the second host compound described in the following Table 1 was used alone as the host of the light-emitting layer.

The driving voltage, current efficiency, and the luminous color at a luminance of 1,000 nit and the time taken for luminance to decrease from 100% to 95% at a luminance of 10,000 nit (lifespan: T₉₅) of the OLEDs of Device Examples 1 to 4 and Device Comparative Example 1 produced as described above, were measured, and the results thereof are shown in the following Table 1.

TABLE 1

| | | | | Current | | |
|---|---|---|---|---|---|---|
| | | Second | Driving | Efficiency | Luminous | Lifespan |
| | First host | host | Voltage | (cd/A) | Color | T₉₅ (h) |
| Device | H1-144 | H2-75 | 3.6 | 34.7 | Red | 41 |
| Example 1 | | | | | | |
| Device | H1-144 | H2-73 | 3.2 | 31.9 | Red | 126 |
| Example 2 | | | | | | |
| Device | H1-144 | H2-5 | 3.2 | 34.5 | Red | 152 |
| Example 3 | | | | | | |
| Device | H1-144 | H2-74 | 3.4 | 35.4 | Red | 177 |
| Example 4 | | | | | | |
| Device | — | H2-5 | 3.6 | 28.0 | Red | 10 |
| Comparative | | | | | | |
| Example 1 | | | | | | |

From Table 1 above, it can be seen that the organic electroluminescent device comprising a specific combination of host materials according to the present disclosure exhibits lower driving voltage and/or higher luminous efficiency compared to the organic electroluminescent device including only a single host material, and in particular, it exhibits significantly improved lifespan characteristics.

The compounds used in Device Examples 1 to 4 and Device Comparative Example 1 are specifically shown in the following Table 2.

TABLE 2

| | |
|---|---|
| Hole Injection Layer/Hole Transport Layer | 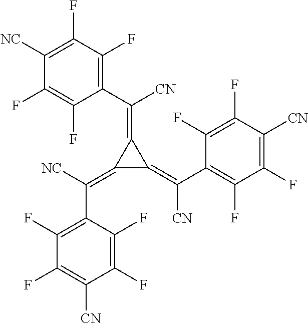 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 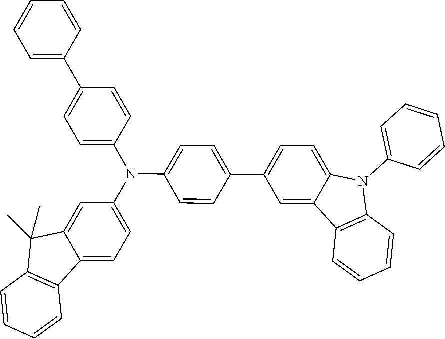 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| | 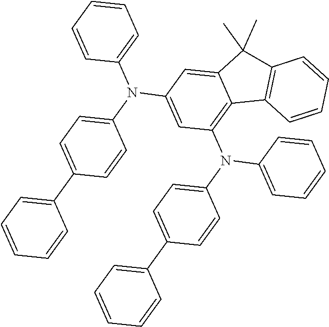 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-2 |
| Light-Emitting Layer | 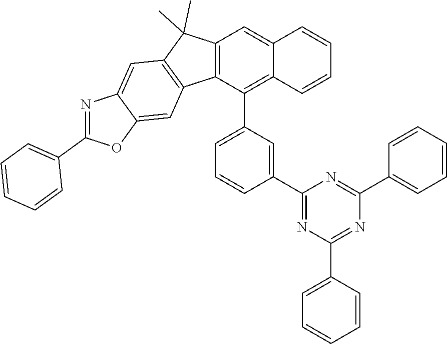 [SMILES: CC1(C)c2cc3nc(-c4ccccc4)oc3cc2-c2c1cc1ccccc1c2-c1cccc(-c2nc(-c3ccccc3)nc(-c3ccccc3)n2)c1] |
| | H2-75 |
| | 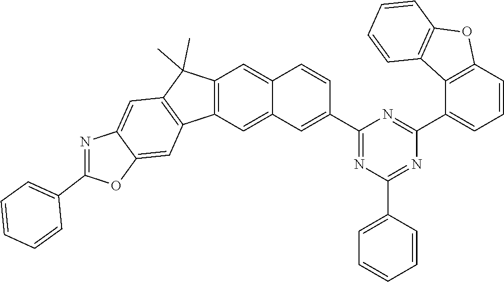 [SMILES: CC1(C)c2cc3ccc(-c4nc(-c5ccccc5)nc(-c5cccc6oc7ccccc7c56)n4)cc3cc2-c2cc3oc(-c4ccccc4)nc3cc21] |
| | H2-73 |
| | 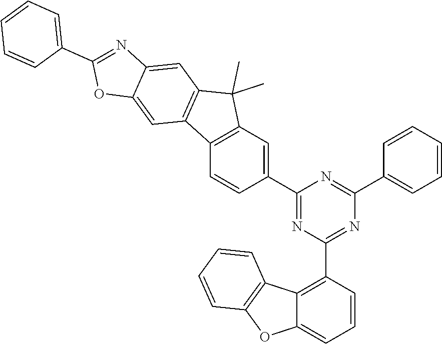 [SMILES: CC1(C)c2cc(-c3nc(-c4ccccc4)nc(-c4cccc5oc6ccccc6c45)n3)ccc2-c2cc3oc(-c4ccccc4)nc3cc21] |
| | H2-5 |
| | 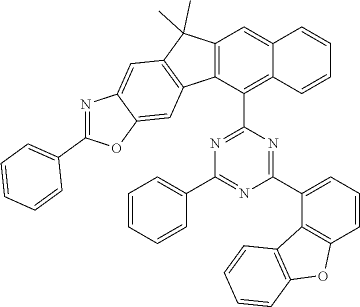 [SMILES: CC1(C)c2cc3nc(-c4ccccc4)oc3cc2-c2c1cc1ccccc1c2-c1nc(-c2ccccc2)nc(-c2cccc3oc4ccccc4c23)n1] |
| | H2-74 |
| | 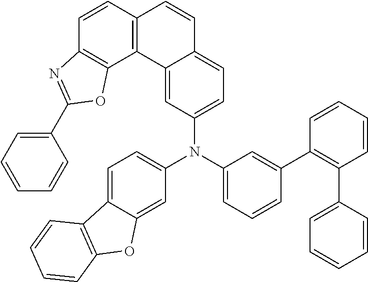 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | H1-144 |
| | 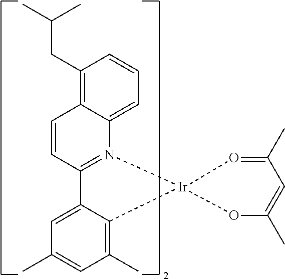 |
| | D-39 |
| Electron Transport Layer/Electron Injection Layer | 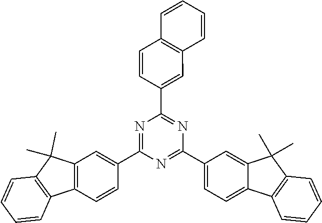 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ET-1 |
| | 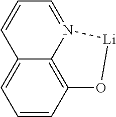 |
| | EI-1 |

### [Device Examples 5 and 6] Preparation of a Blue Light-Emitting Organic Electroluminescent Device Comprising the Organic Electroluminescent Compound According to the Present Disclosure as an Electron Buffer Layer Material

OLEDs according to the present disclosure were produced. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropanol and then used. The ITO substrate was mounted on a substrate holder of a vacuum vapor deposition apparatus. Then, compound HI-2 was introduced into one cell of the vacuum vapor deposition apparatus. After evacuating until the degree of vacuum in the chamber reaches 10⁻⁷ Torr, it was evaporated by applying an electric current to the cell, thereby forming a first hole injection layer having a thickness of 60 nm on the ITO substrate. Next, compound HI-3 was introduced into another cell of the vacuum vapor deposition apparatus and was evaporated by applying an electric current to the cell, thereby forming a second hole injection layer having a thickness of 5 nm on the first hole injection layer. Then, compound HT-1 was introduced into another cell of the vacuum vapor deposition apparatus, and was evaporated by applying an electric current to the cell, thereby forming a first hole transport layer having a thickness of 20 nm on the second hole injection layer. Next, compound HT-3 was introduced into another cell of the vacuum vapor deposition apparatus and was evaporated by applying an electric current to the cell, thereby forming a second hole transport layer having a thickness of 5 nm on the first hole transport layer. After forming the hole injection layers and the hole transport layers, a light-emitting layer was formed thereon as follows: Compound BH-1 was introduced into one cell of the vacuum vapor deposition apparatus as a host, and compound BD-1 was introduced into another cell as a dopant. The two materials were evaporated at different rates and the dopant was deposited in a doping amount of 2 wt % based on the total amount of the host and dopant to form a light-emitting layer having a thickness of 20 nm on the second hole transport layer. Next, the compound listed in Table 3 was deposited as an electron buffer material having a thickness of 5 nm on the light-emitting layer, and then compound ET-1 as an electron transport material was introduced into one cell and evaporated to deposit an electron transport layer having a thickness of 30 nm. Next, after depositing compound EI-1 as an electron injection layer having a thickness of 2 nm on the electron transport layer, an AI cathode having a thickness of 80 nm was deposited by another vacuum vapor deposition apparatus. Thus, OLEDs were produced. Each compound used for all the materials were purified by vacuum sublimation under 10⁻⁶ Torr.

### [Device Comparative Example 2] Preparation of a Blue Light-Emitting Organic Electroluminescent Device Containing a Comparative Compound as an Electron Buffer Layer Material

An OLED was manufactured in the same manner as Device Example 5, except that the electron buffer material shown in the following Table 3 was used as the electron buffer material.

The driving voltage, luminous efficiency, and the luminous color at a luminance of 1,000 nit of the OLEDs of Device Examples 5 and 6 and Device Comparative Example 2 produced as described above, were measured, and the results thereof are shown in the following Table 3.

TABLE 3

| | Electron | Driving | Luminous | |
|---|---|---|---|---|
| | Buffer | Voltage | Efficiency | Luminous |
| | Material | (V) | (cd/A) | Color |
| Device Example 5 | H2-75 | 3.9 | 6.3 | Blue |
| Device Example 6 | H2-74 | 4.8 | 5.0 | Blue |
| Device Comparative | H2-5 | 4.9 | 4.5 | Blue |
| Example 2 | | | | |

From Table 3 above, it can be seen that the organic electroluminescent device containing the compound according to the present disclosure as an electron buffer material exhibits a low driving voltage and/or high luminous efficiency compared to an organic electroluminescent device using a comparative compound as an electron buffer material.

The compounds used in Device Examples 5 and 6 and Comparative Example 2 above are shown in the following Table 4.

TABLE 4

| | |
|---|---|
| Hole Injection Layer/Hole Transport Layer | 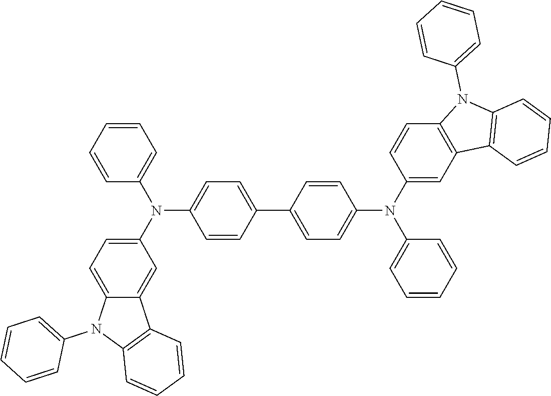 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1] |
| | HI-2 |
| | 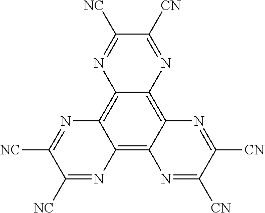 [SMILES: N#Cc1nc2c3nc(C#N)c(C#N)nc3c3nc(C#N)c(C#N)nc3c2nc1C#N] |
| | HI-3 |
| | 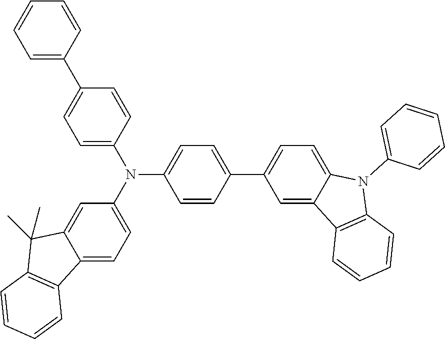 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| | 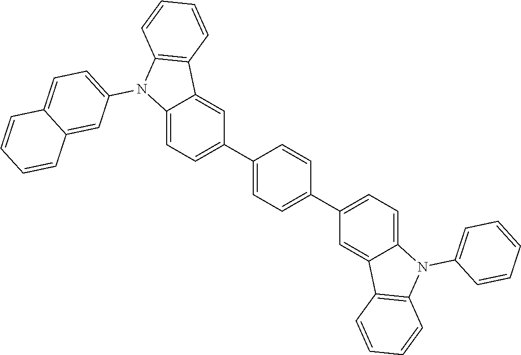 [SMILES: c1ccc(-n2c3ccccc3c3cc(-c4ccc(-c5ccc6c(c5)c5ccccc5n6-c5ccc6ccccc6c5)cc4)ccc32)cc1] |
| | HT-3 |
| Light-Emitting Layer | 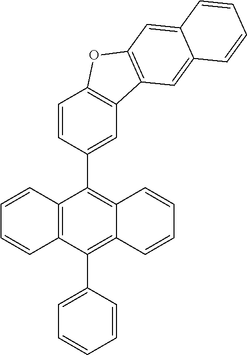 [SMILES: c1ccc(-c2c3ccccc3c(-c3ccc4oc5cc6ccccc6cc5c4c3)c3ccccc23)cc1] |
| | BH-1 |
| | 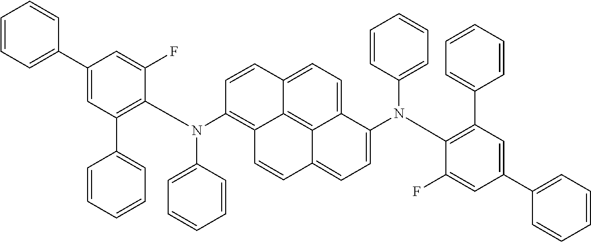 [SMILES: Fc1cc(-c2ccccc2)cc(-c2ccccc2)c1N(c1ccccc1)c1ccc2ccc3c(N(c4ccccc4)c4c(F)cc(-c5ccccc5)cc4-c4ccccc4)ccc4ccc1c2c43] |
| | BD-1 |
| Electron Buffer Layer/Electron Transport Layer/ Electron Injection Layer | 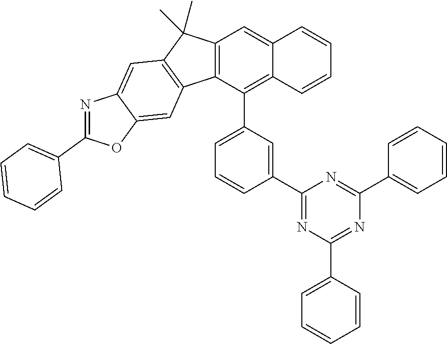 [SMILES: CC1(C)c2cc3nc(-c4ccccc4)oc3cc2-c2c1cc1ccccc1c2-c1cccc(-c2nc(-c3ccccc3)nc(-c3ccccc3)n2)c1] |
| | H2-75 |
| | 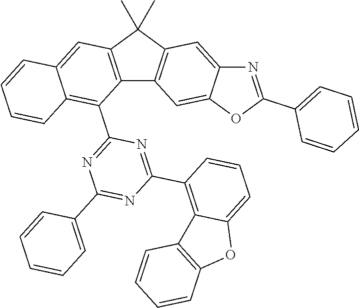 [SMILES: CC1(C)c2cc3nc(-c4ccccc4)oc3cc2-c2c1cc1ccccc1c2-c1nc(-c2ccccc2)nc(-c2cccc3oc4ccccc4c23)n1] |
| | H2-74 |
| | 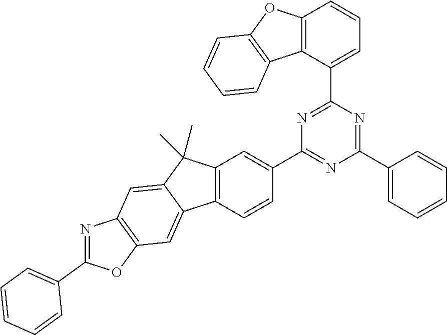 [SMILES: CC1(C)c2cc(-c3nc(-c4ccccc4)nc(-c4cccc5oc6ccccc6c45)n3)ccc2-c2cc3oc(-c4ccccc4)nc3cc21] |
| | H2-5 |
| | 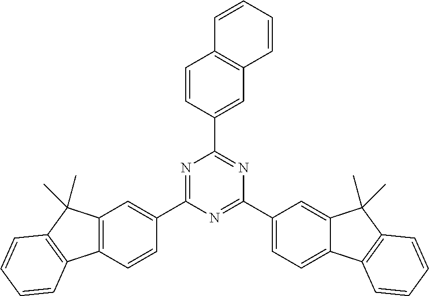 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ET-1 |
| | 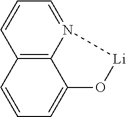 |
| | EI-1 |

1. A plurality of host materials comprising a first host material comprising a compound represented by the following Formula 1 and a second host material comprising a compound represented by the following Formula 2: 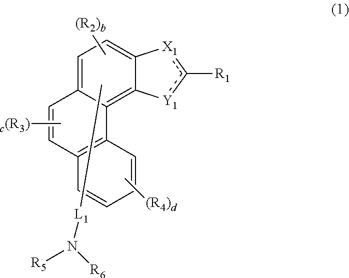
wherein,
X₁ and Y₁ each independently represent —N=, —NR₇, —O—, or —S—; provided that any one of X₁ and Y₁ is —N=, and the other of X₁ and Y₁ is —NR₇—, —O—, or —S—;
R₁ represents a substituted or unsubstituted (C₆-C₃₀)aryl or a substituted or unsubstituted (3- to 30-membered)heteroaryl;
R₂ to R₄ and R₇ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and (C₆-C₃₀) aromatic ring, or —L₁₁N (Ar₁)(Ar₂); or may be linked to the adjacent substituents to form a ring(s);
L₁ and L₁₁ each independently represent a single bond, a substituted or unsubstituted (C₆-C₃₀)arylene, or a substituted or unsubstituted (3- to 30-membered)heteroarylene;
Ar₁ and Ar₂ each independently represent hydrogen, a substituted or unsubstituted (C₁-C₃₀)alkyl, a substituted or unsubstituted (C₂-C₃₀)alkenyl, a substituted or unsubstituted fused ring of (C₃-C₃₀) aliphatic ring and (C₆-C₃₀) aromatic ring, a substituted or unsubstituted (C₆-C₃₀)aryl, or a substituted or unsubstituted (3- to 30-membered)heteroaryl;
R₅ and R₆ each independently represent a substituted or unsubstituted (C₆-C₃₀)aryl or a substituted or unsubstituted (3- to 30-membered)heteroaryl; and
b and c each independently represent an integer of 1 or 2, d represents an integer of 1 to 4, and when b to d are an integer of 2 or more, each of R₂ to R₄ may be the same or different; 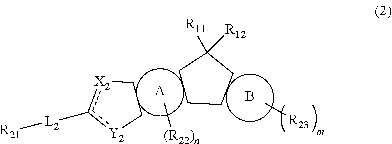
wherein,
ring A and ring B each independently represent a substituted or unsubstituted (C₆-C₃₀)arene or a substituted or unsubstituted (3- to 30-membered)heteroarene;
R₁₁ and R₁₂ each independently represent a substituted or unsubstituted (C₁-C₃₀)alkyl, a substituted or unsubstituted (C₆-C₃₀)aryl, or a substituted or unsubstituted (3-to 30-membered)heteroaryl; or may be linked to each other to form a ring(s);
X₂ and Y₂ each independently represent —N=, —NR₂₄, —O—, or —S—; provided that any one of X₂ and Y₂ is —N=, and the other of X₂ and Y₂ is —NR₂₄—, —O—, or —S—;
R₂₁ to R₂₄ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and a (C₆-C₃₀) aromatic ring, or —L₃-HAr;
provided that at least one of R₂₁ to R₂₄ is —L₃-HAr;
L₂ and L₃ each independently represent a single bond, a substituted or unsubstituted (C₆-C₃₀)arylene, or a substituted or unsubstituted (3- to 30-membered)heteroarylene;
HAr represents a substituted or unsubstituted (3- to 30-membered)heteroaryl containing at least one nitrogen atom; and
n represents an integer of 1 to 4, m represents an integer of 1 to 8, and when n and m are an integer of 2 or more, each of R₂₂ and R₂₃ may be the same or different.

2. The plurality of host materials according to claim 1, wherein the substituted alkyl, the substituted aryl(ene), the substituted heteroaryl(ene), the substituted cycloalkyl, the substituted alkoxy, the substituted trialkylsilyl, the substituted dialkylarylsilyl, the substituted alkyldiarylsilyl, the substituted triarylsilyl, and the substituted fused ring of aliphatic ring and aromatic ring each independently are substituted with at least one selected from the group consisting of deuterium, halogen, cyano, carboxyl, nitro, hydroxyl, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (5- to 30-membered)heteroaryl unsubstituted or substituted with (C₆-C₃₀)aryl, (C₆-C₃₀)aryl unsubstituted or substituted with (5- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, a fused ring of (C₃-C₃₀) aliphatic ring and a (C₆-C₃₀) aromatic ring, amino, mono- or di(C₁-C₃₀)alkylamino, a substituted or unsubstituted mono- or di(C₆-C₃₀)arylamino, (C₁-C₃₀)alkyl(C₆-C₃₀)arylamino, mono- or di(3- to 30-membered)heteroarylamino, (C₁-C₃₀)alkyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, (C₆-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, and (C₁-C₃₀)alkyl(C₆-C₃₀)aryl.

3. The plurality of host materials according to claim 1, wherein HAr in Formula 2 is represented by the following Formula 3 or 4: 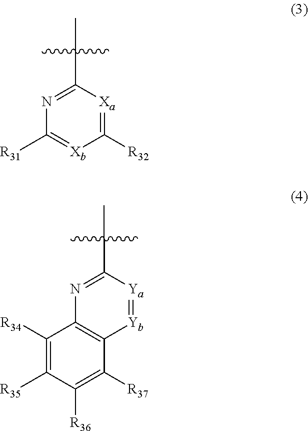
wherein,
Xₐ, X_{b}, Yₐ, and Y_{b} each independently represent N or CR₃₃; provided that at least one of Xₐ and X_{b} is N, and at least one of Yₐ and Y_{b} is N; and
R₃₁ to R₃₇ each independently represent hydrogen, deuterium, halogen, cyano, a substituted or unsubstituted (C₁-C₃₀)alkyl, a substituted or unsubstituted (C₆-C₅₀)aryl, a substituted or unsubstituted (3- to 50-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, or a substituted or unsubstituted tri(C₆-C₃₀)arylsilyl; or may be linked to the adjacent substituents to form a ring(s).

4. The plurality of host materials according to claim 1, wherein Formula 2 is represented by any one of the following Formulas 2-1 to 2-8: 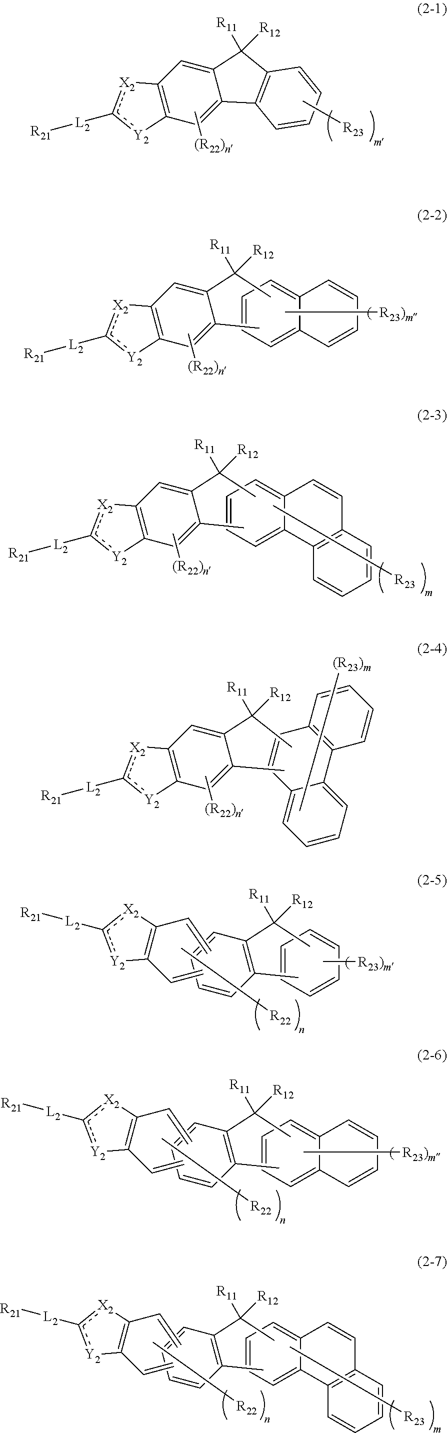 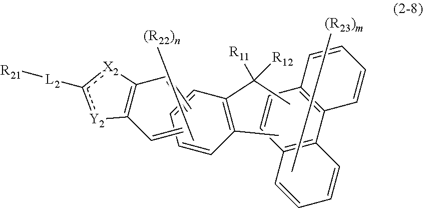
wherein,
X₂, Y₂, R₁₁, R₁₂, R₂₁ to R₂₃, L₂, n, and m are as defined in claim 1; and
n′ represents an integer of 1 or 2, m′ represents an integer of 1 to 4, and m″ represents an integer of 1 to 6.

5. The plurality of host materials according to claim 1, wherein Formula 2 is represented by any one of the following Formulas 2-1 and 2-9: 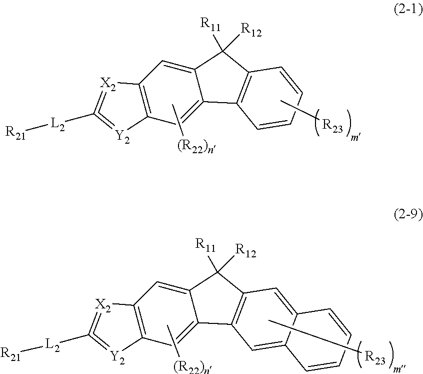
wherein,
X₂, Y₂, R₁₁ and R₁₂, R₂₁ to R₂₃, and L₂ are as defined in claim 1; and
n′ represents an integer of 1 or 2, m′ represents an integer of 1 to 4, and m″ represents an integer of 1 to 6.

6. The plurality of host materials according to claim 1, wherein Formula 1 is represented by any one of the following Formulas 1-1 to 1-4: 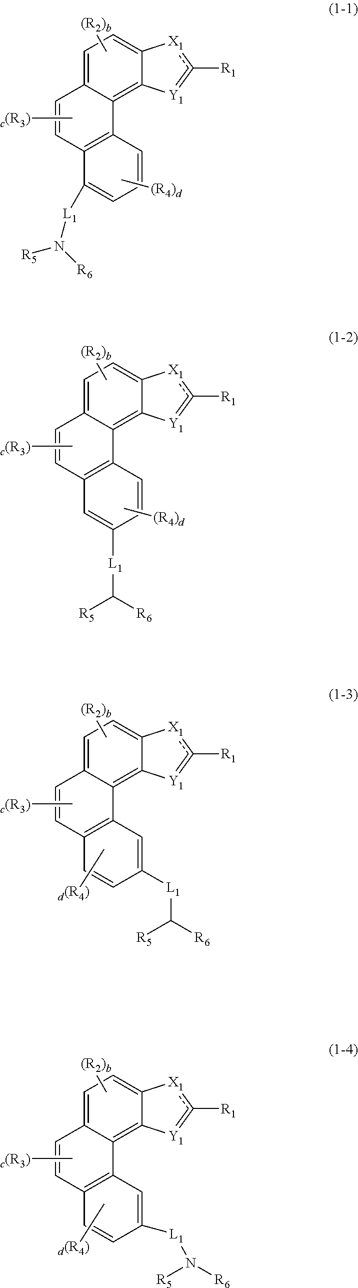
wherein,
d represents an integer of 1 to 3, and when d is an integer of 2 or more, each of R₄ may be the same or different; and
X₁, Y₁, R₁ to R₆, L₁, b, and c are as defined in claim 1.

7. The plurality of host materials according to claim 1, wherein the compound represented by Formula 1 is selected from the following compounds:

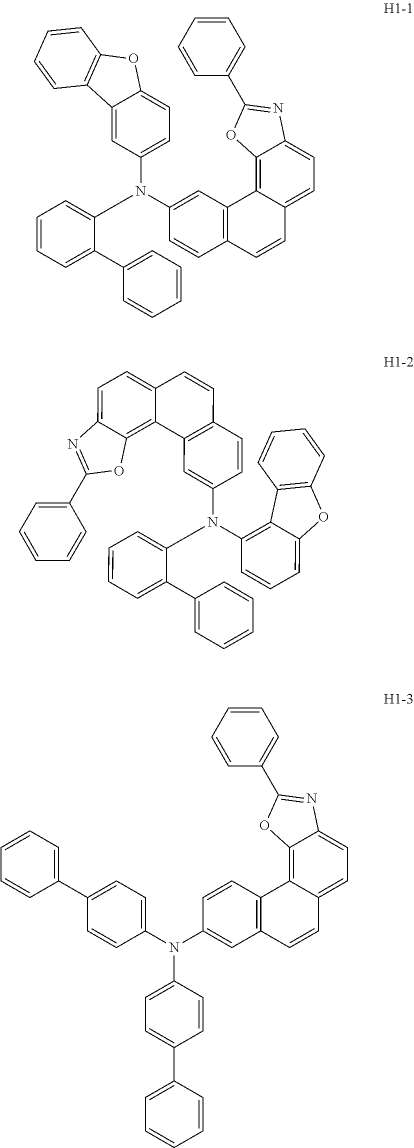 [SMILES: c1ccc(-c2nc3ccc4ccc5ccc(N(c6ccc7oc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

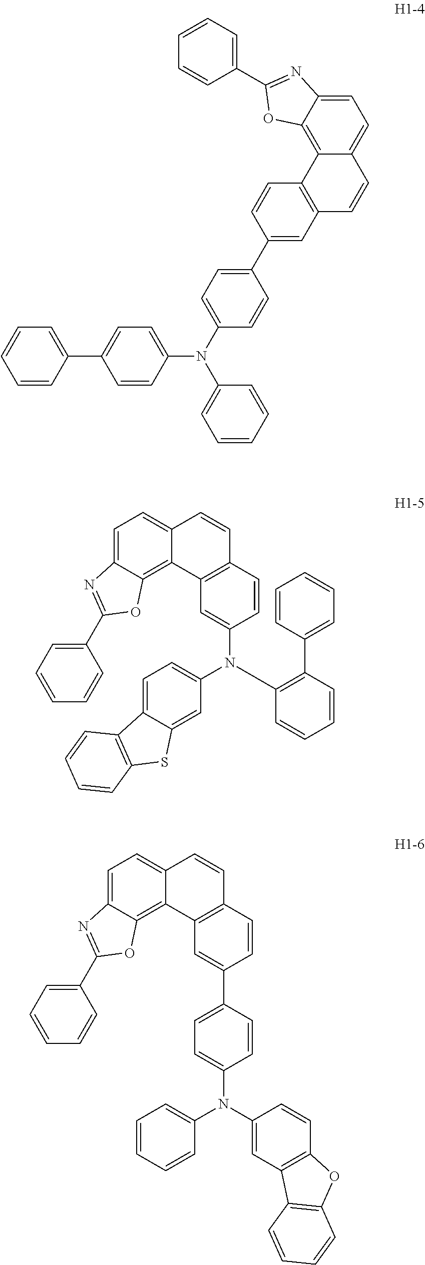 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

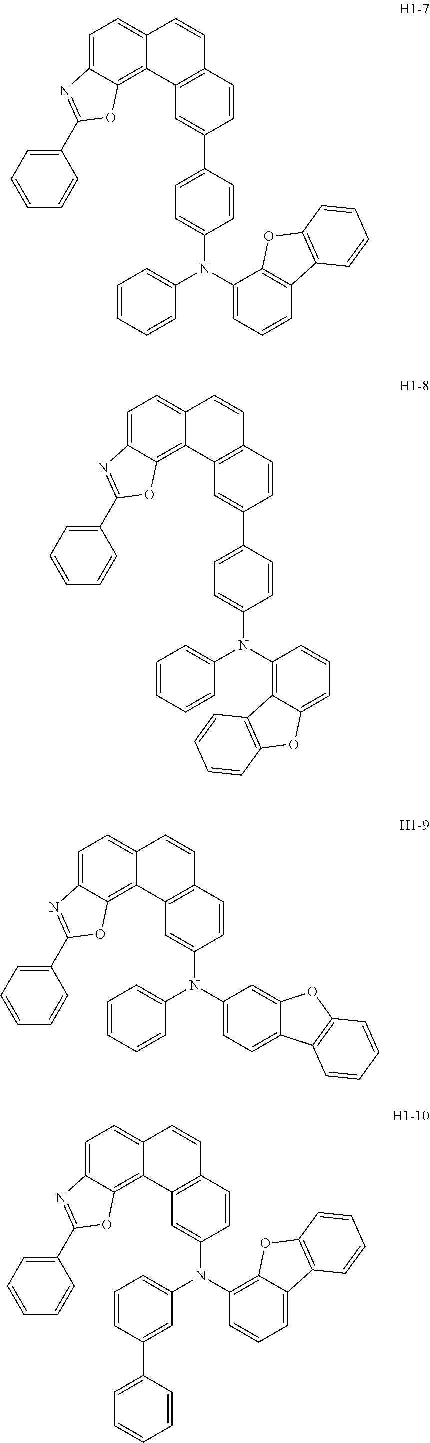 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccccc6)c6ccc7c(c6)oc6ccccc67)cc5c4c3o2)cc1 ; c1ccc(-c2cccc(N(c3ccc4ccc5ccc6nc(-c7ccccc7)oc6c5c4c3)c3cccc4c3oc3ccccc34)c2)cc1]

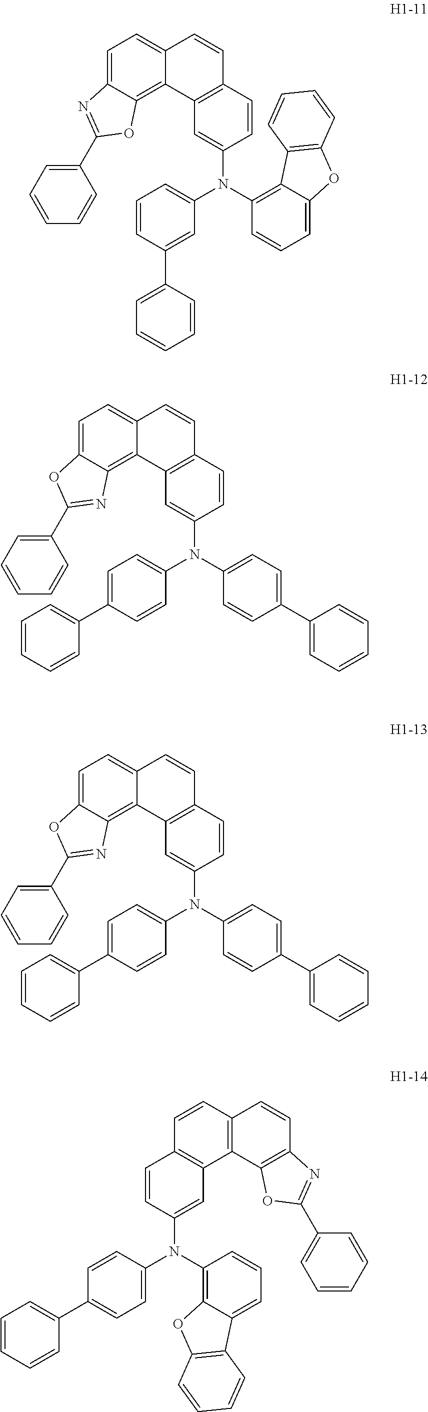 [SMILES: c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc4ccc5ccc6nc(-c7ccccc7)oc6c5c4c3)c3cccc4c3oc3ccccc34)cc2)cc1]

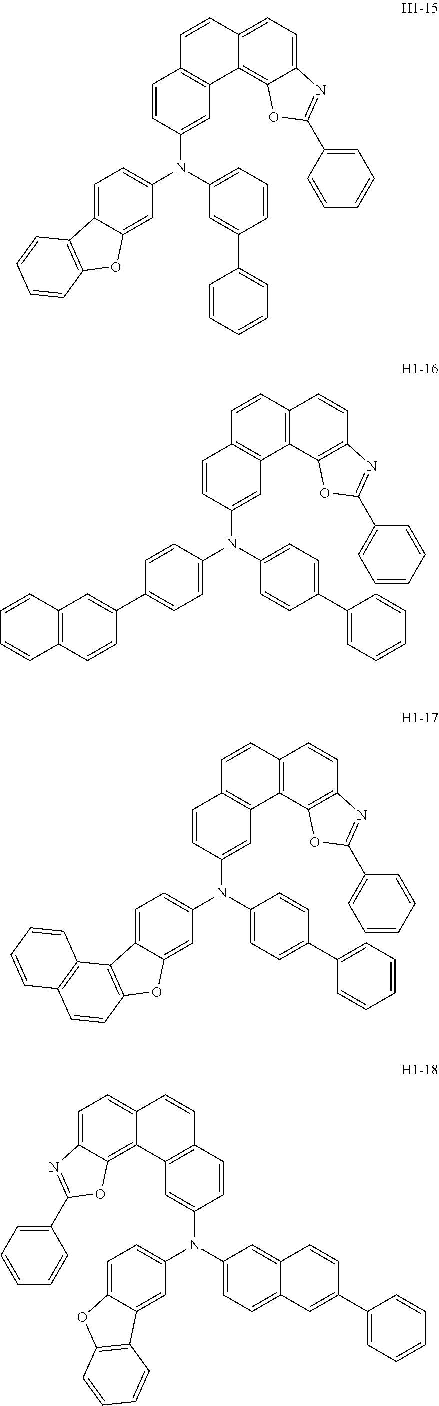 [SMILES: c1ccc(-c2cccc(N(c3ccc4c(c3)oc3ccccc34)c3ccc4ccc5ccc6nc(-c7ccccc7)oc6c5c4c3)c2)cc1 ; c1ccc(-c2ccc(N(c3ccc(-c4ccc5ccccc5c4)cc3)c3ccc4ccc5ccc6nc(-c7ccccc7)oc6c5c4c3)cc2)cc1 ; c1ccc(-c2ccc(N(c3ccc4c(c3)oc3ccc5ccccc5c34)c3ccc4ccc5ccc6nc(-c7ccccc7)oc6c5c4c3)cc2)cc1 ; c1ccc(-c2ccc3cc(N(c4ccc5oc6ccccc6c5c4)c4ccc5ccc6ccc7nc(-c8ccccc8)oc7c6c5c4)ccc3c2)cc1]

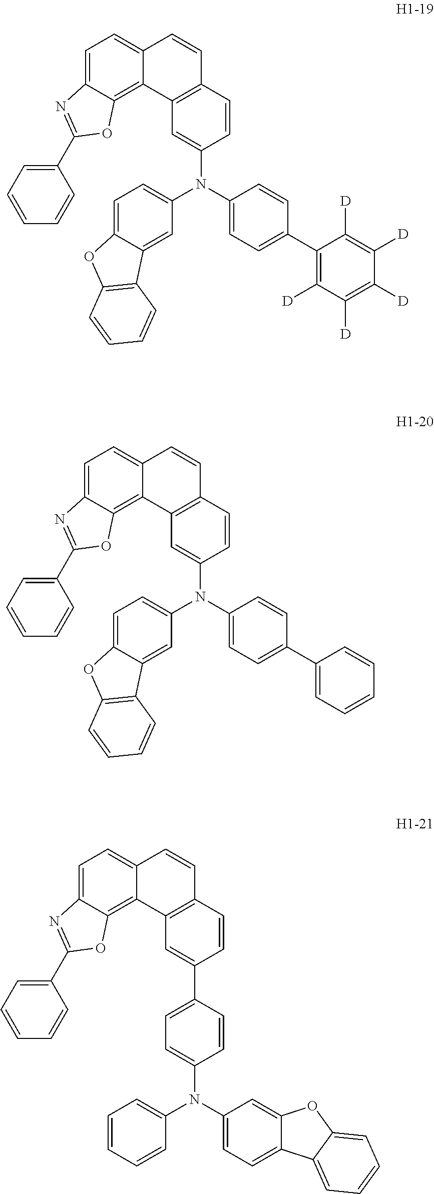 [SMILES: [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2nc3ccc4ccc5ccc(-c6ccc(N(c7ccccc7)c7ccc8c(c7)oc7ccccc78)cc6)cc5c4c3o2)cc1]

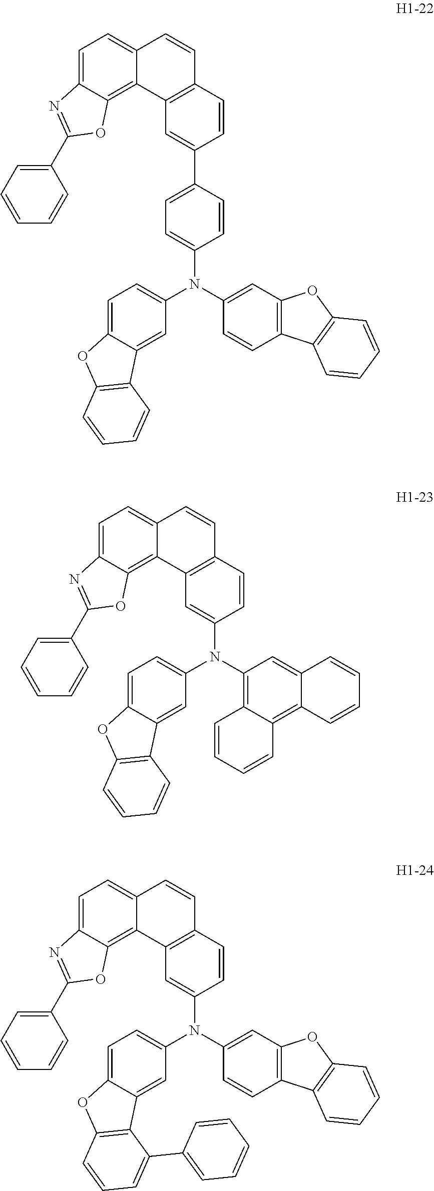 [SMILES: c1ccc(-c2nc3ccc4ccc5ccc(-c6ccc(N(c7ccc8c(c7)oc7ccccc78)c7ccc8oc9ccccc9c8c7)cc6)cc5c4c3o2)cc1 ; c1ccc(-c2nc3ccc4ccc5ccc(N(c6ccc7oc8ccccc8c7c6)c6cc7ccccc7c7ccccc67)cc5c4c3o2)cc1 ; c1ccc(-c2nc3ccc4ccc5ccc(N(c6ccc7c(c6)oc6ccccc67)c6ccc7oc8cccc(-c9ccccc9)c8c7c6)cc5c4c3o2)cc1]

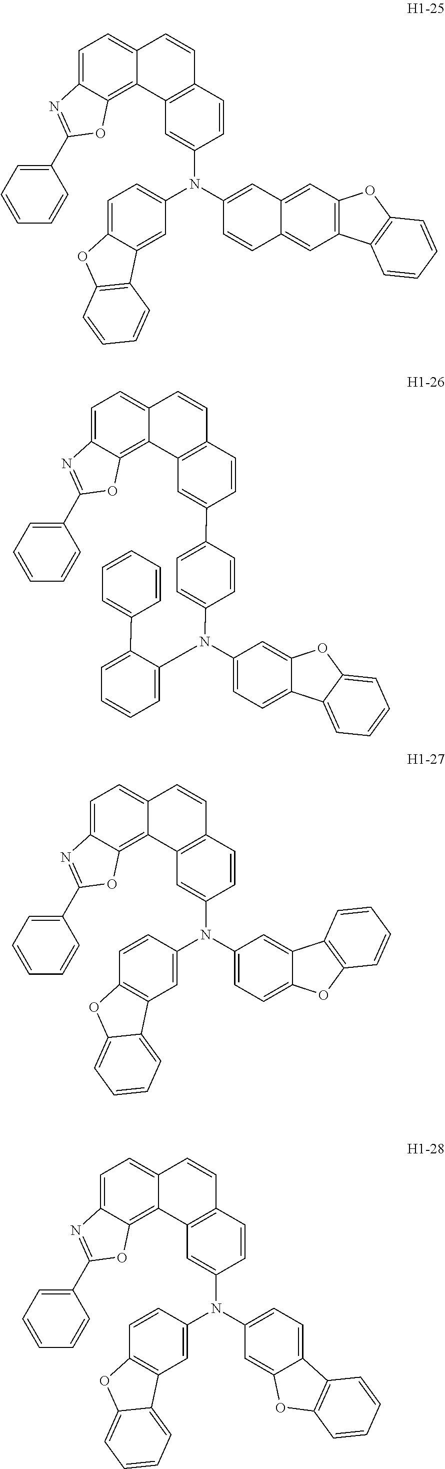 [SMILES: c1ccc(-c2nc3ccc4ccc5ccc(N(c6ccc7cc8c(cc7c6)oc6ccccc68)c6ccc7oc8ccccc8c7c6)cc5c4c3o2)cc1 ; c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1]

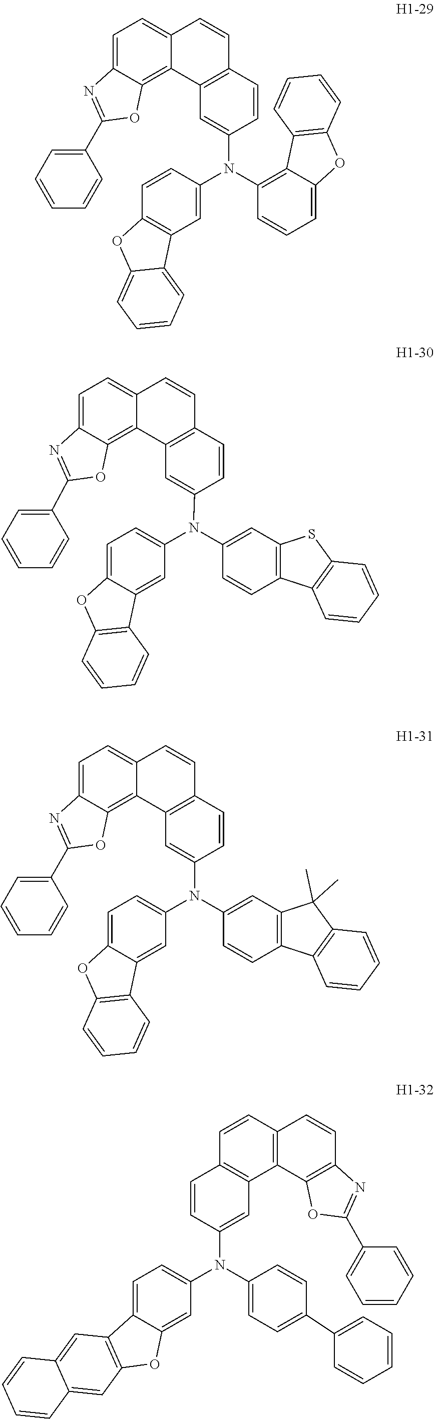 [SMILES: c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21 ; c1ccc(-c2ccc(N(c3ccc4c(c3)oc3cc5ccccc5cc34)c3ccc4ccc5ccc6nc(-c7ccccc7)oc6c5c4c3)cc2)cc1]

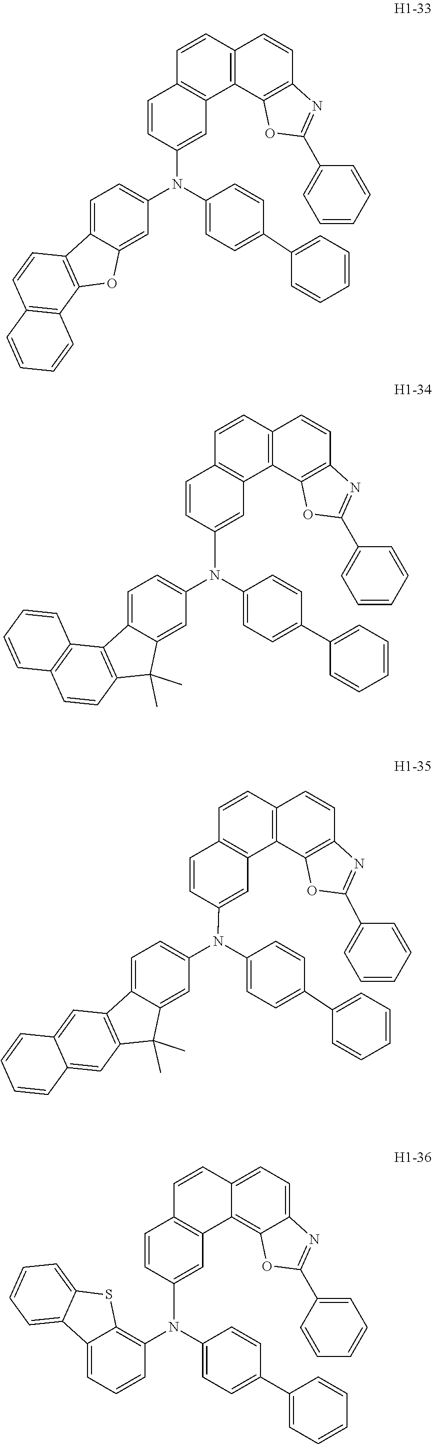 [SMILES: c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21 ; c1ccc(-c2ccc(N(c3ccc4ccc5ccc6nc(-c7ccccc7)oc6c5c4c3)c3cccc4c3sc3ccccc34)cc2)cc1]

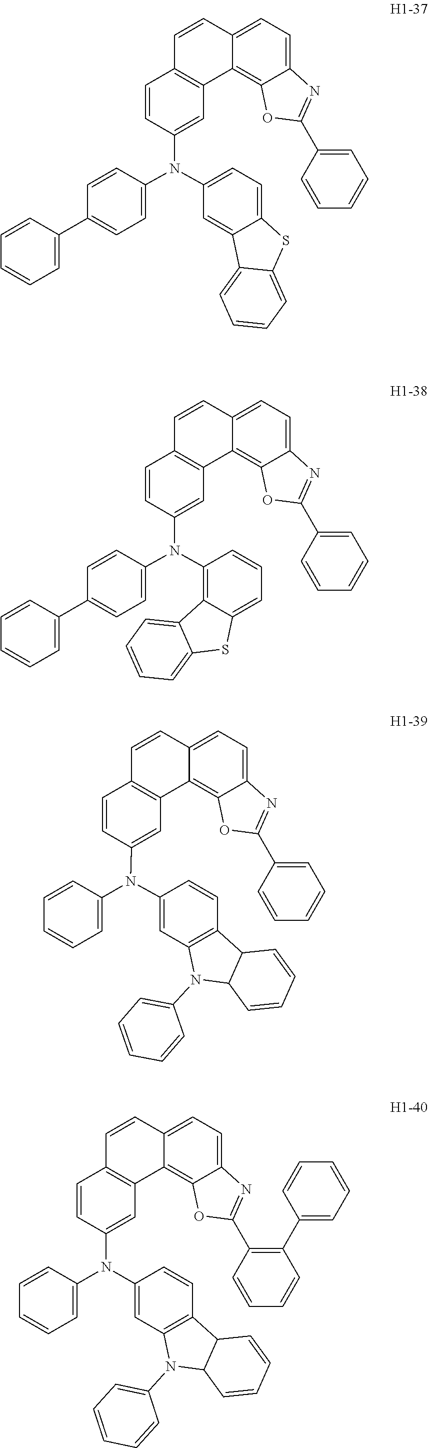 [SMILES: c1ccc(-c2ccc(N(c3ccc4sc5ccccc5c4c3)c3ccc4ccc5ccc6nc(-c7ccccc7)oc6c5c4c3)cc2)cc1 ; c1ccc(-c2ccc(N(c3ccc4ccc5ccc6nc(-c7ccccc7)oc6c5c4c3)c3cccc4sc5ccccc5c34)cc2)cc1 ; C1=CC2c3ccc(N(c4ccccc4)c4ccc5ccc6ccc7nc(-c8ccccc8)oc7c6c5c4)cc3N(c3ccccc3)C2C=C1 ; C1=CC2c3ccc(N(c4ccccc4)c4ccc5ccc6ccc7nc(-c8ccccc8-c8ccccc8)oc7c6c5c4)cc3N(c3ccccc3)C2C=C1]

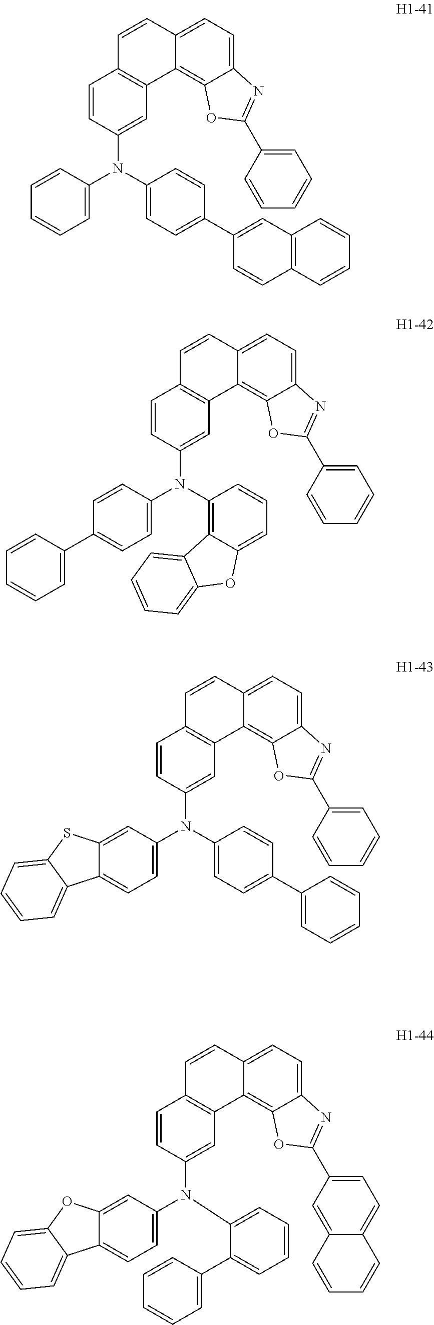 [SMILES: c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1 ; c1ccc(-c2ccccc2N(c2ccc3c(c2)oc2ccccc23)c2ccc3ccc4ccc5nc(-c6ccc7ccccc7c6)oc5c4c3c2)cc1]

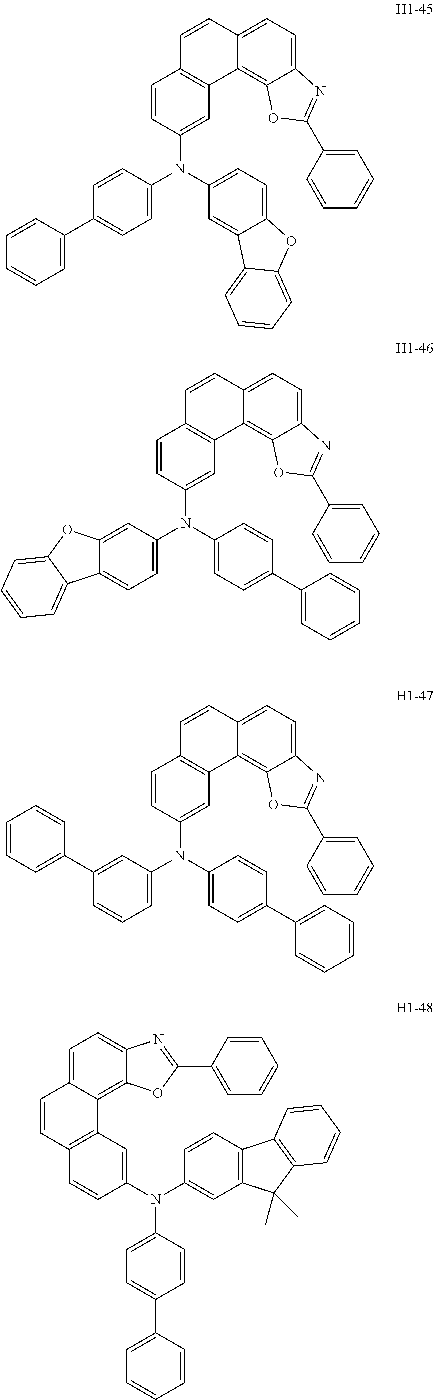

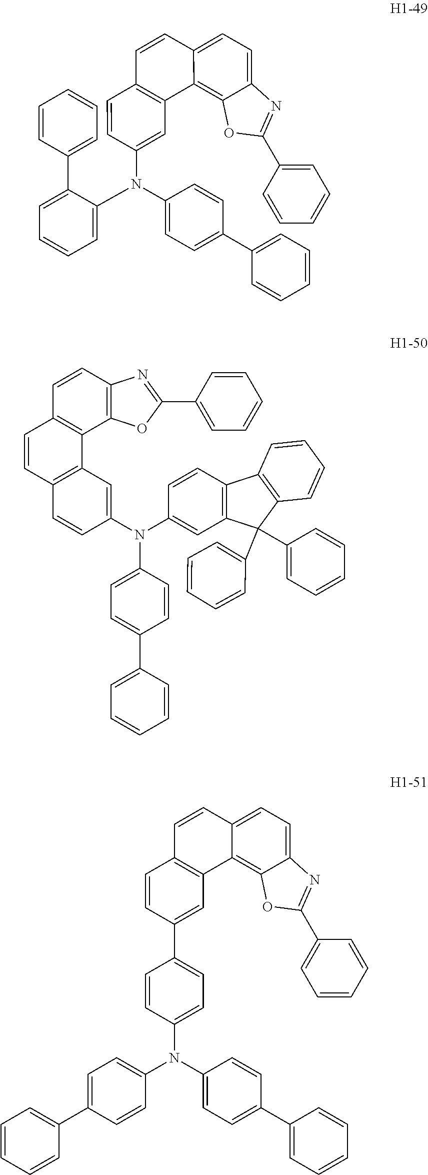 [SMILES: c1ccc(-c2ccc(N(c3ccc4ccc5ccc6nc(-c7ccccc7)oc6c5c4c3)c3ccccc3-c3ccccc3)cc2)cc1 ; c1ccc(-c2ccc(N(c3ccc4c(c3)C(c3ccccc3)(c3ccccc3)c3ccccc3-4)c3ccc4ccc5ccc6nc(-c7ccccc7)oc6c5c4c3)cc2)cc1 ; c1ccc(-c2ccc(N(c3ccc(-c4ccccc4)cc3)c3ccc(-c4ccc5ccc6ccc7nc(-c8ccccc8)oc7c6c5c4)cc3)cc2)cc1]

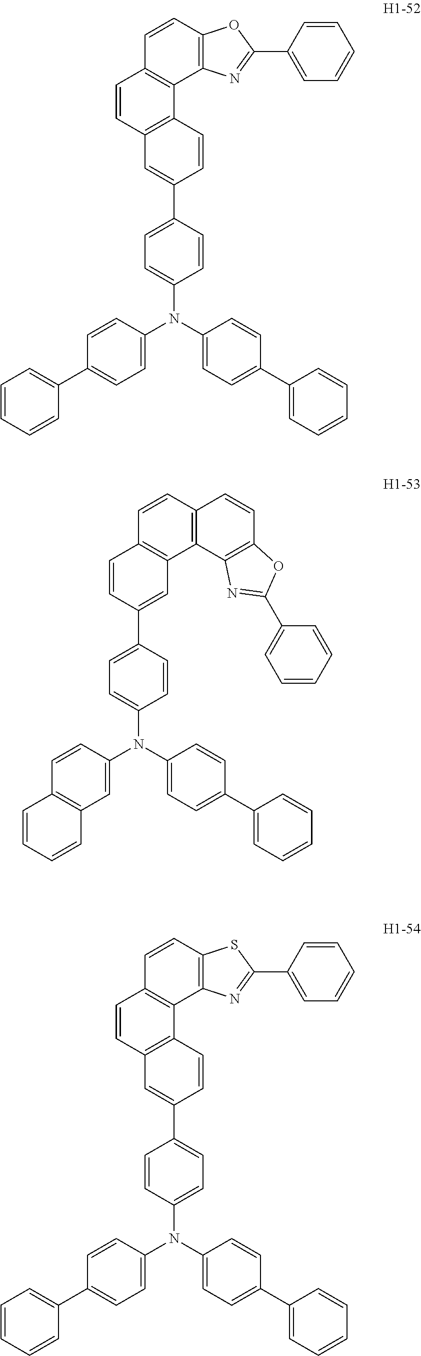 [SMILES: c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1 ; c1ccc(-c2ccc(N(c3ccc(-c4ccccc4)cc3)c3ccc(-c4ccc5c(ccc6ccc7sc(-c8ccccc8)nc7c65)c4)cc3)cc2)cc1]

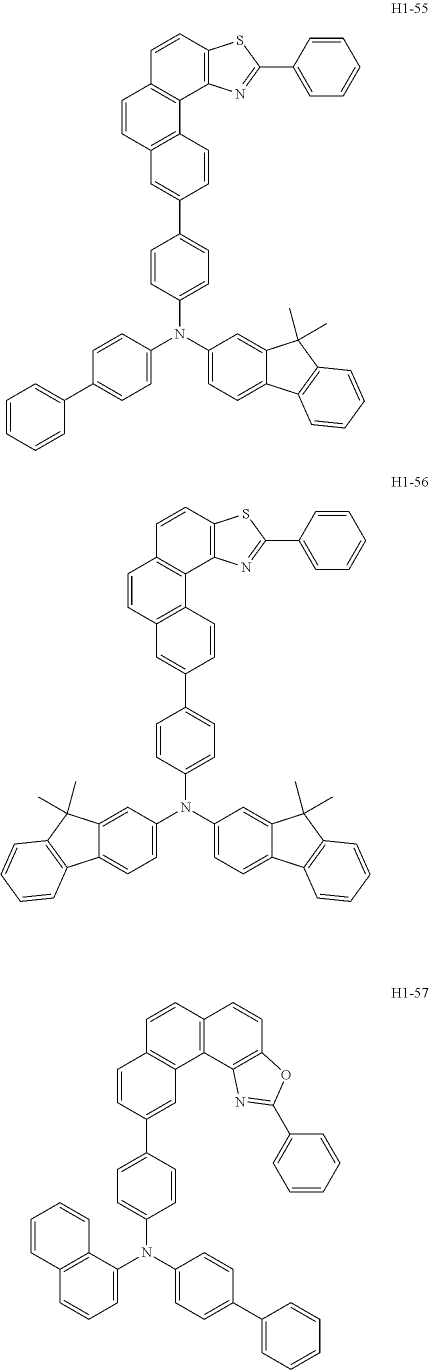 [SMILES: CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21 ; c1ccc(-c2ccc(N(c3ccc(-c4ccc5ccc6ccc7oc(-c8ccccc8)nc7c6c5c4)cc3)c3cccc4ccccc34)cc2)cc1]

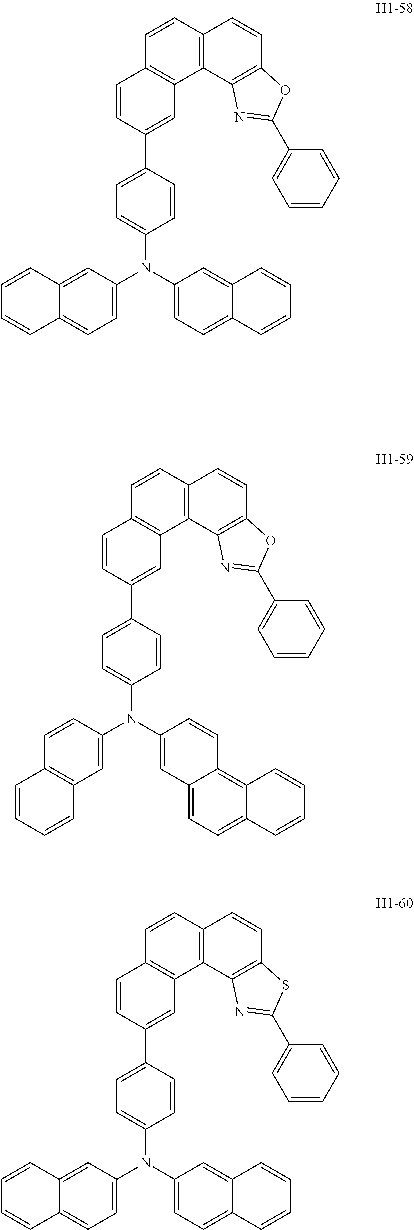 [SMILES: c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1 ; c1ccc(-c2nc3c(ccc4ccc5ccc(-c6ccc(N(c7ccc8ccccc8c7)c7ccc8ccccc8c7)cc6)cc5c43)s2)cc1]

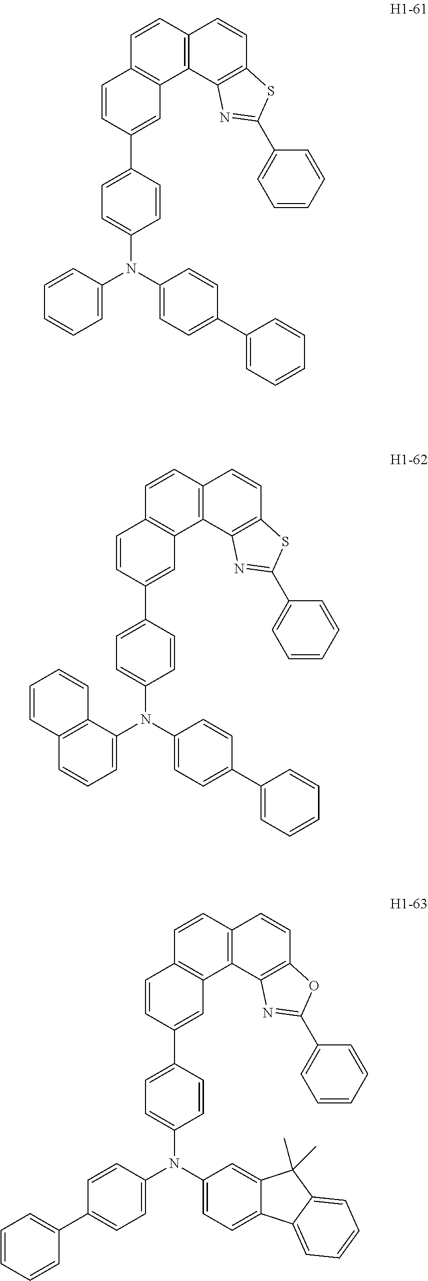 [SMILES: c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1 ; CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21]

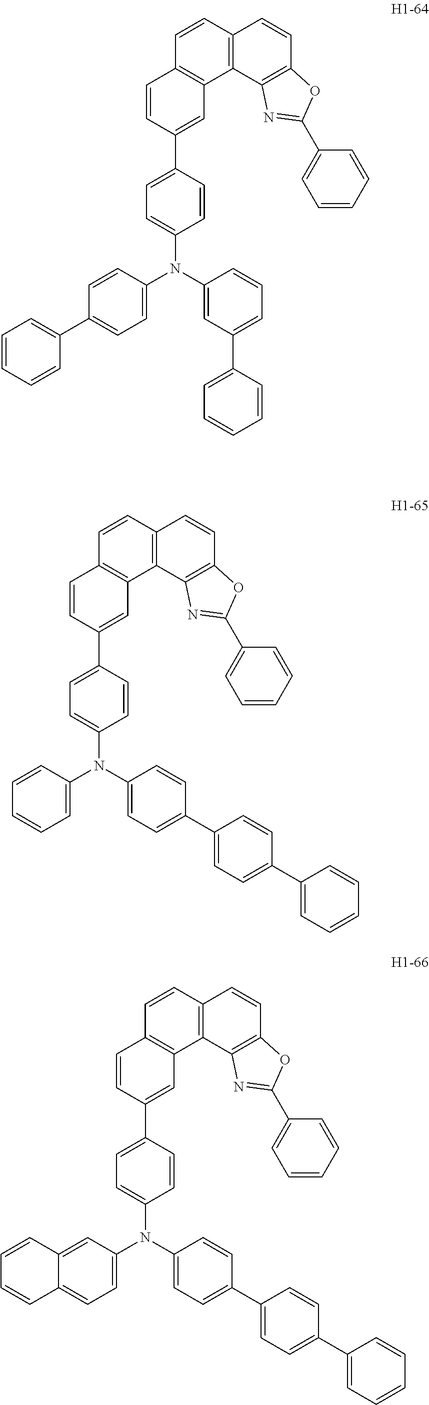 [SMILES: c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1 ; c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1]

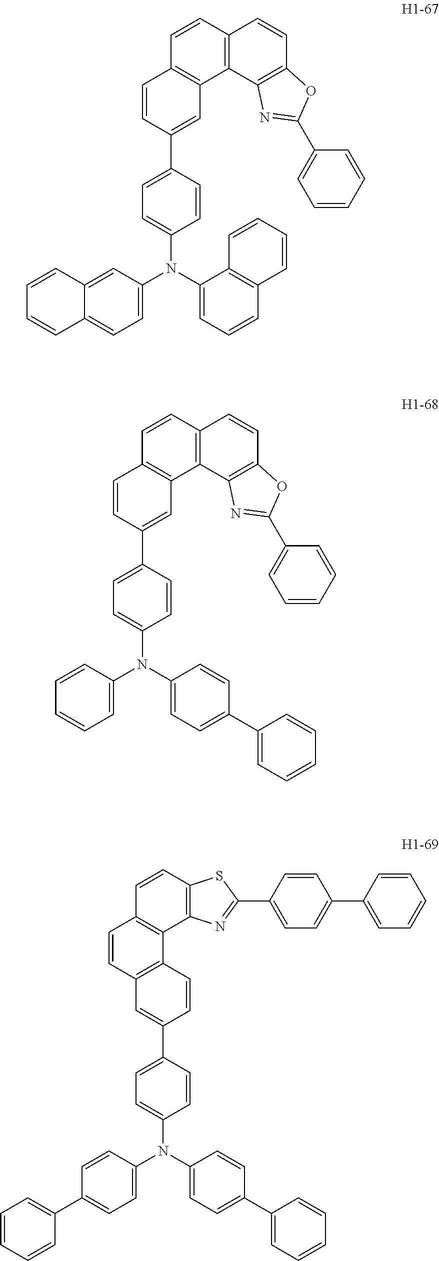 [SMILES: c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1 ; c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1]

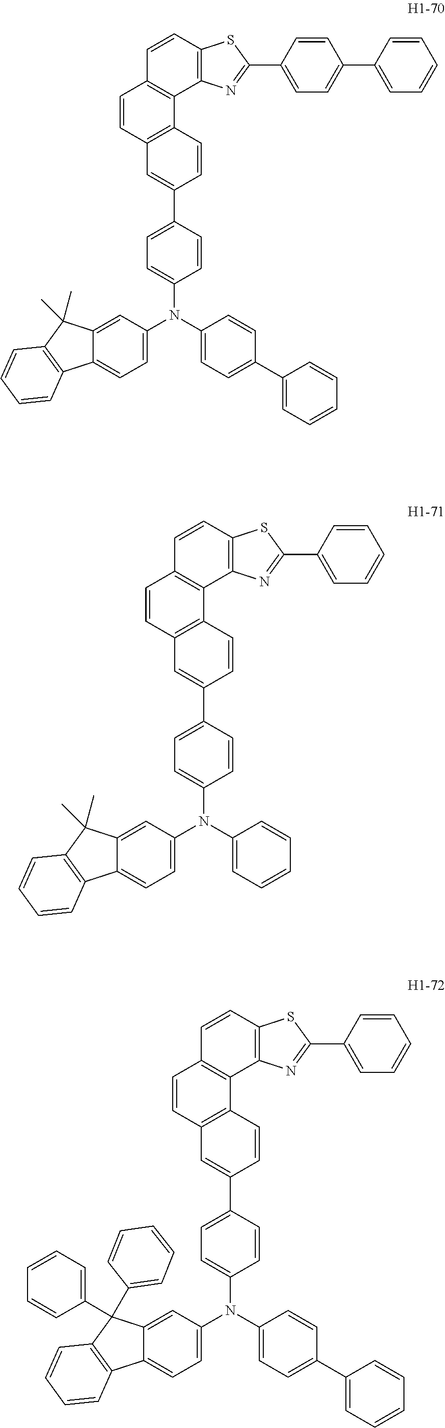 [SMILES: CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21 ; c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1]

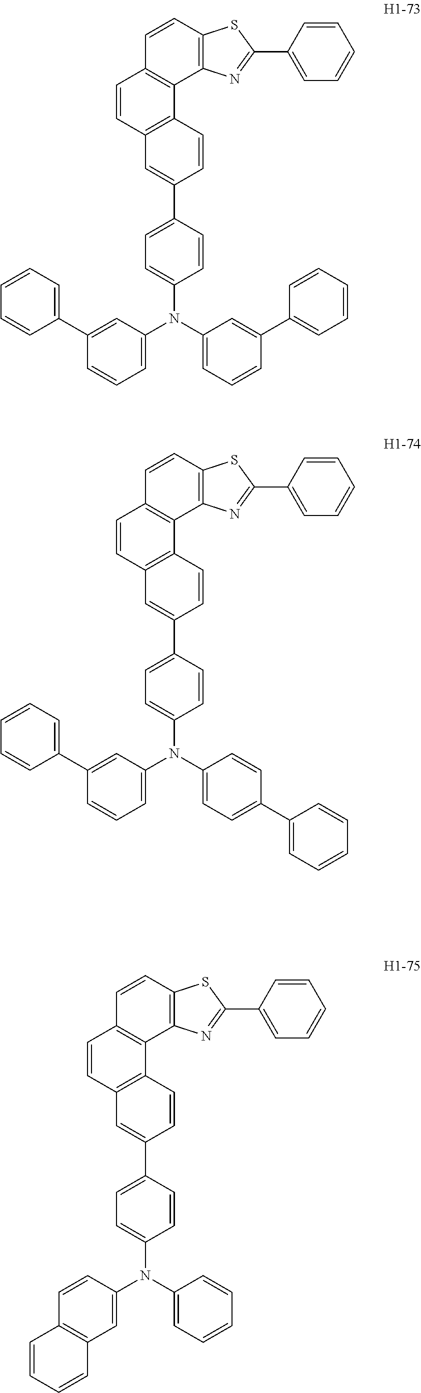 [SMILES: c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1 ; c1ccc(-c2nc3c(ccc4ccc5cc(-c6ccc(N(c7ccccc7)c7ccc8ccccc8c7)cc6)ccc5c43)s2)cc1]

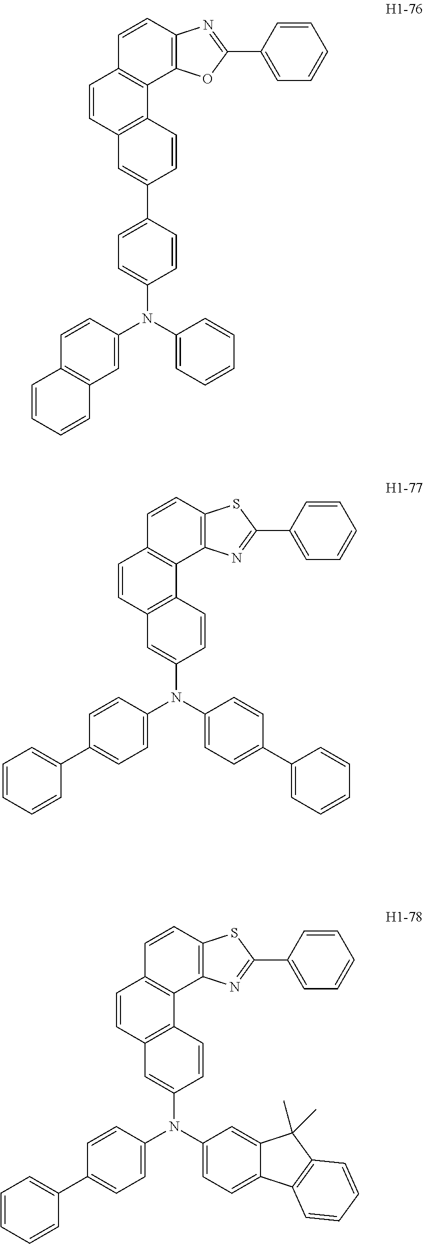 [SMILES: c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1 ; CC1(C)c2ccccc2-c2ccc(N(c3ccc(-c4ccccc4)cc3)c3ccc4c(ccc5ccc6sc(-c7ccccc7)nc6c54)c3)cc21]

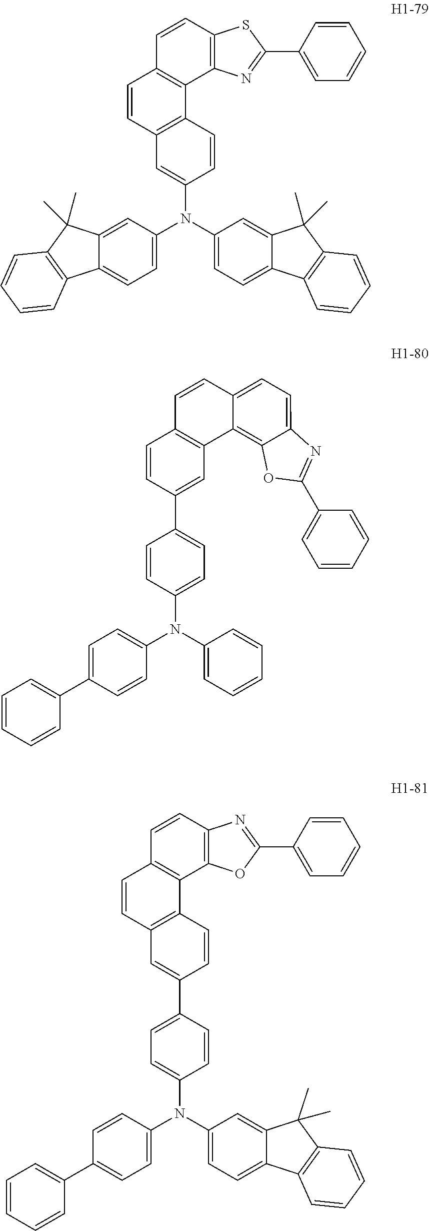 [SMILES: CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1 ; CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21]

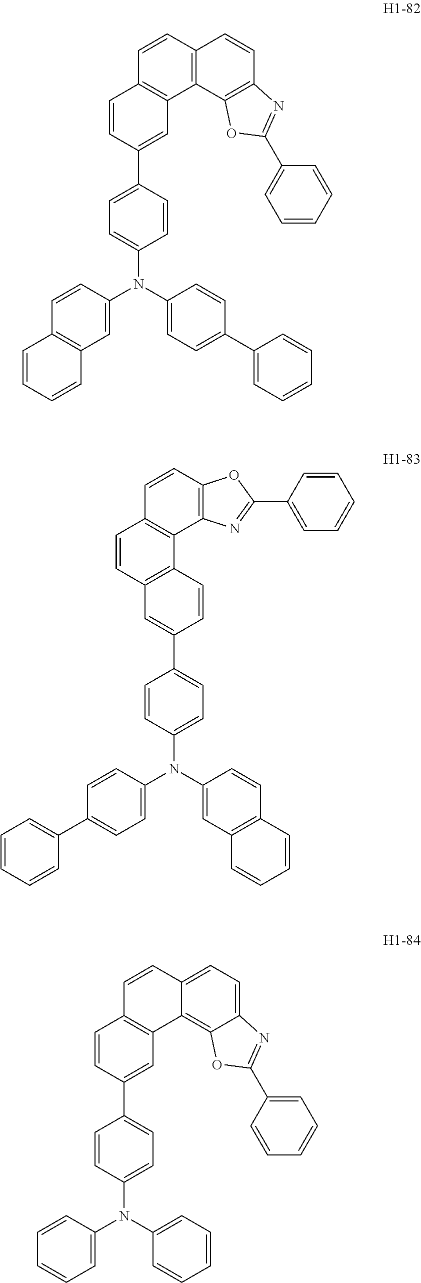 [SMILES: c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1 ; c1ccc(-c2nc3ccc4ccc5ccc(-c6ccc(N(c7ccccc7)c7ccccc7)cc6)cc5c4c3o2)cc1]

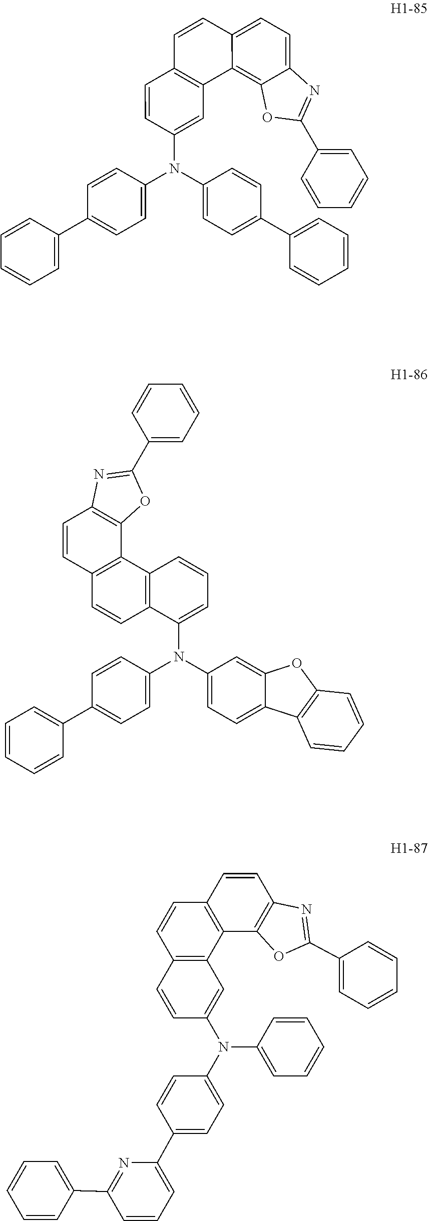 [SMILES: c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1 ; c1ccc(-c2cccc(-c3ccc(N(c4ccccc4)c4ccc5ccc6ccc7nc(-c8ccccc8)oc7c6c5c4)cc3)n2)cc1]

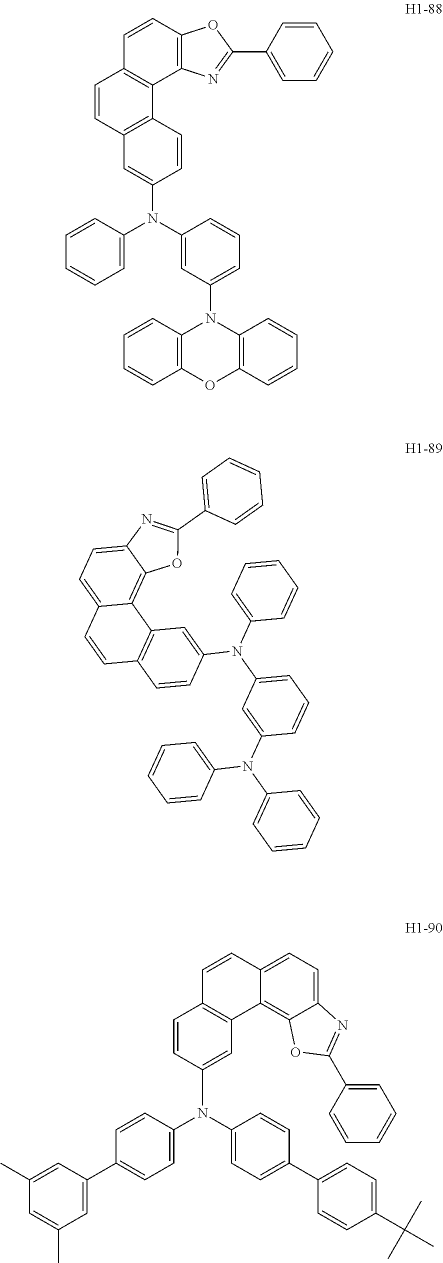 [SMILES: c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1 ; Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1]

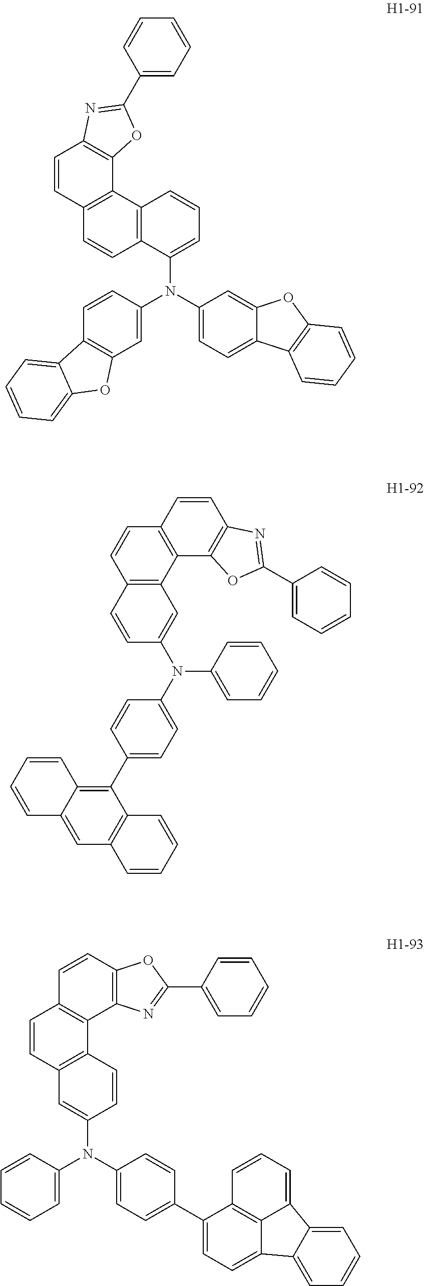 [SMILES: c1ccc(-c2nc3ccc4ccc5c(N(c6ccc7c(c6)oc6ccccc67)c6ccc7c(c6)oc6ccccc67)cccc5c4c3o2)cc1 ; c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1]

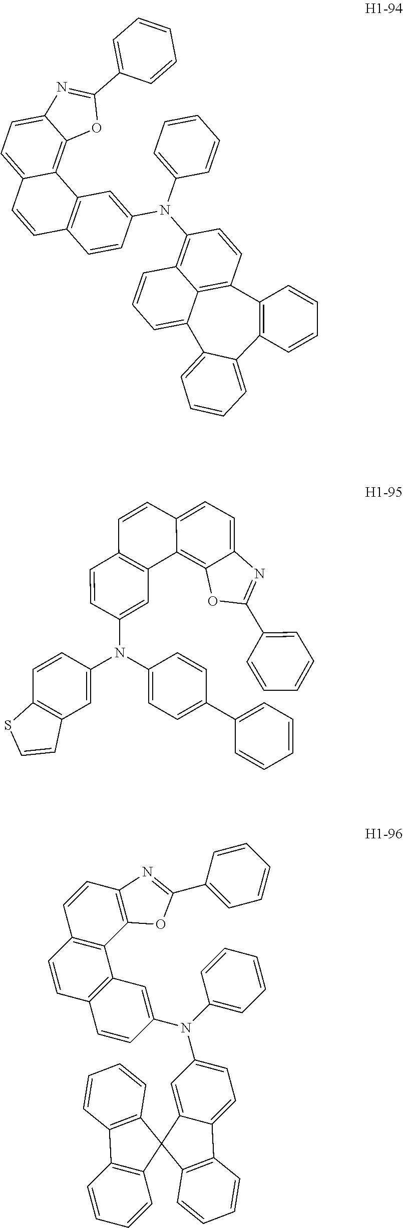 [SMILES: c1ccc(-c2nc3ccc4ccc5ccc(N(c6ccccc6)c6ccc7c8c(cccc68)-c6ccccc6-c6ccccc6-7)cc5c4c3o2)cc1 ; c1ccc(-c2ccc(N(c3ccc4sccc4c3)c3ccc4ccc5ccc6nc(-c7ccccc7)oc6c5c4c3)cc2)cc1 ; c1ccc(-c2nc3ccc4ccc5ccc(N(c6ccccc6)c6ccc7c(c6)C6(c8ccccc8-c8ccccc86)c6ccccc6-7)cc5c4c3o2)cc1]

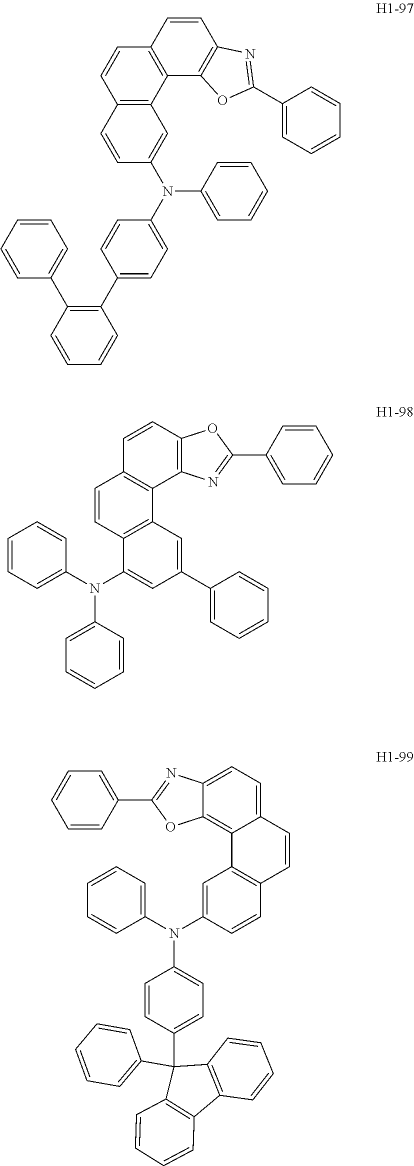 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1]

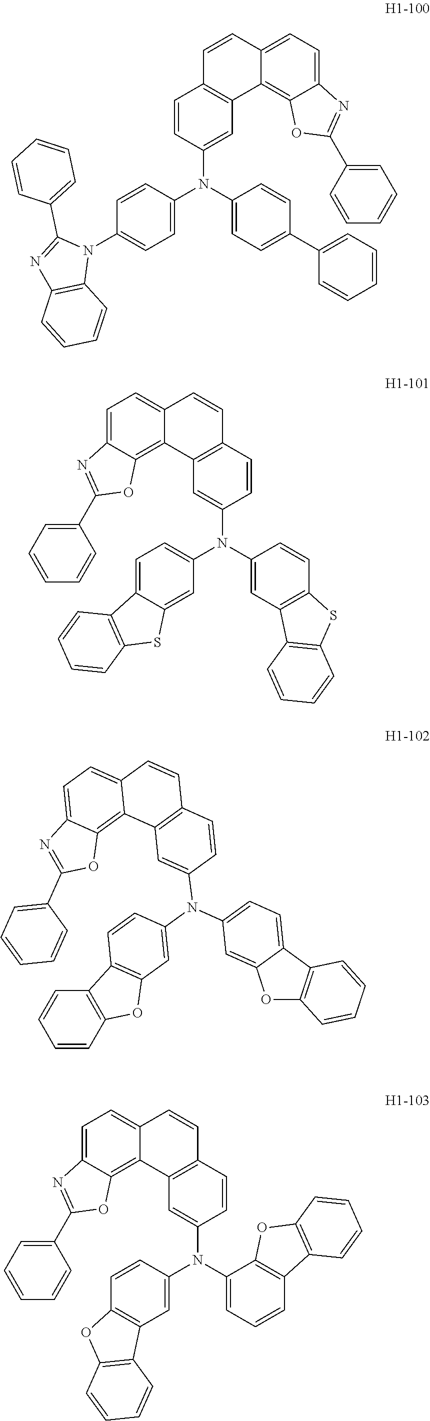 [SMILES: c1ccc(-c2ccc(N(c3ccc(-n4c(-c5ccccc5)nc5ccccc54)cc3)c3ccc4ccc5ccc6nc(-c7ccccc7)oc6c5c4c3)cc2)cc1 ; c1ccc(-c2nc3ccc4ccc5ccc(N(c6ccc7c(c6)sc6ccccc67)c6ccc7sc8ccccc8c7c6)cc5c4c3o2)cc1 ; c1ccc(-c2nc3ccc4ccc5ccc(N(c6ccc7c(c6)oc6ccccc67)c6ccc7c(c6)oc6ccccc67)cc5c4c3o2)cc1 ; c1ccc(-c2nc3ccc4ccc5ccc(N(c6ccc7oc8ccccc8c7c6)c6cccc7c6oc6ccccc67)cc5c4c3o2)cc1]

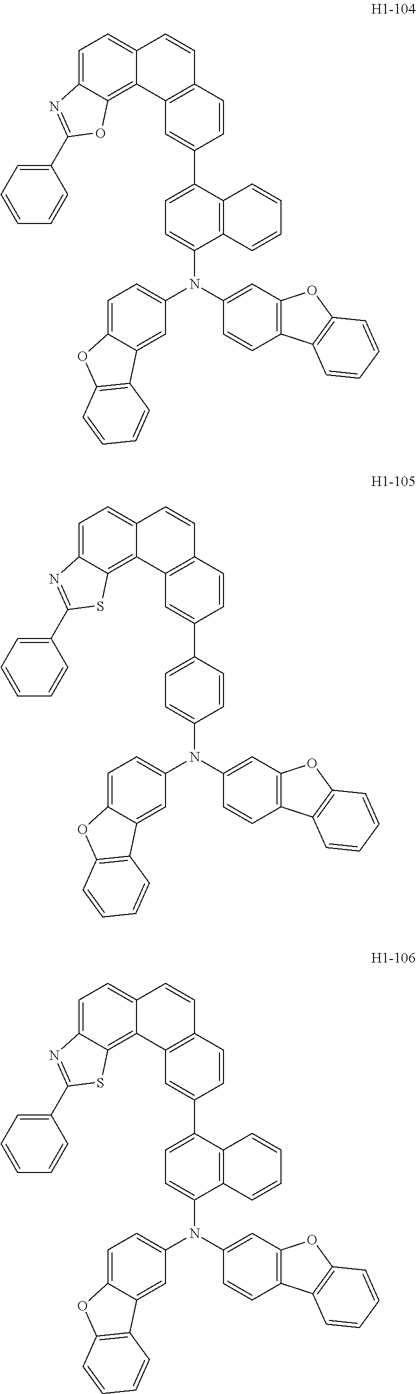 [SMILES: c1ccc(-c2nc3ccc4ccc5ccc(-c6ccc(N(c7ccc8c(c7)oc7ccccc78)c7ccc8oc9ccccc9c8c7)c7ccccc67)cc5c4c3o2)cc1 ; c1ccc(-c2nc3ccc4ccc5ccc(-c6ccc(N(c7ccc8c(c7)oc7ccccc78)c7ccc8oc9ccccc9c8c7)cc6)cc5c4c3s2)cc1 ; c1ccc(-c2nc3ccc4ccc5ccc(-c6ccc(N(c7ccc8c(c7)oc7ccccc78)c7ccc8oc9ccccc9c8c7)c7ccccc67)cc5c4c3s2)cc1]

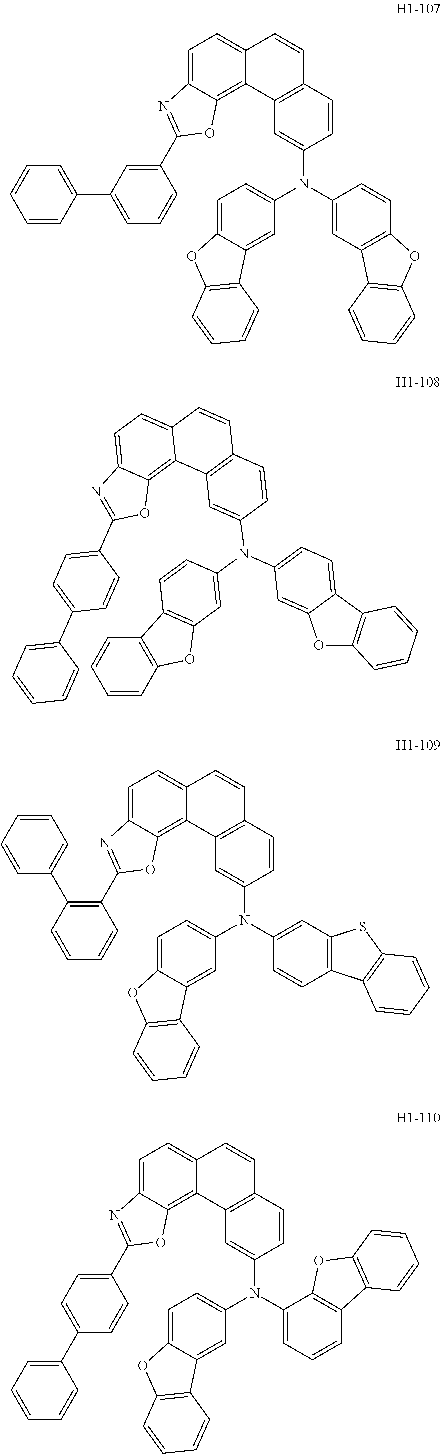 [SMILES: c1ccc(-c2cccc(-c3nc4ccc5ccc6ccc(N(c7ccc8oc9ccccc9c8c7)c7ccc8oc9ccccc9c8c7)cc6c5c4o3)c2)cc1 ; c1ccc(-c2ccc(-c3nc4ccc5ccc6ccc(N(c7ccc8c(c7)oc7ccccc78)c7ccc8c(c7)oc7ccccc78)cc6c5c4o3)cc2)cc1 ; c1ccc(-c2ccccc2-c2nc3ccc4ccc5ccc(N(c6ccc7c(c6)sc6ccccc67)c6ccc7oc8ccccc8c7c6)cc5c4c3o2)cc1 ; c1ccc(-c2ccc(-c3nc4ccc5ccc6ccc(N(c7ccc8oc9ccccc9c8c7)c7cccc8c7oc7ccccc78)cc6c5c4o3)cc2)cc1]

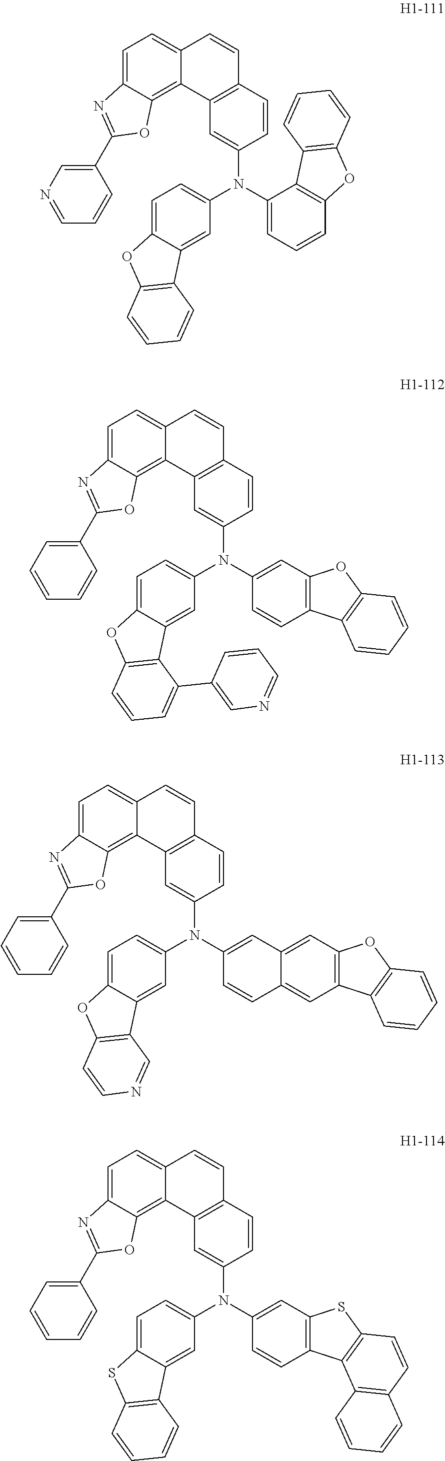 [SMILES: c1cncc(-c2nc3ccc4ccc5ccc(N(c6ccc7oc8ccccc8c7c6)c6cccc7oc8ccccc8c67)cc5c4c3o2)c1 ; c1ccc(-c2nc3ccc4ccc5ccc(N(c6ccc7c(c6)oc6ccccc67)c6ccc7oc8cccc(-c9cccnc9)c8c7c6)cc5c4c3o2)cc1 ; c1ccc(-c2nc3ccc4ccc5ccc(N(c6ccc7cc8c(cc7c6)oc6ccccc68)c6ccc7oc8ccncc8c7c6)cc5c4c3o2)cc1 ; c1ccc(-c2nc3ccc4ccc5ccc(N(c6ccc7c(c6)sc6ccc8ccccc8c67)c6ccc7sc8ccccc8c7c6)cc5c4c3o2)cc1]

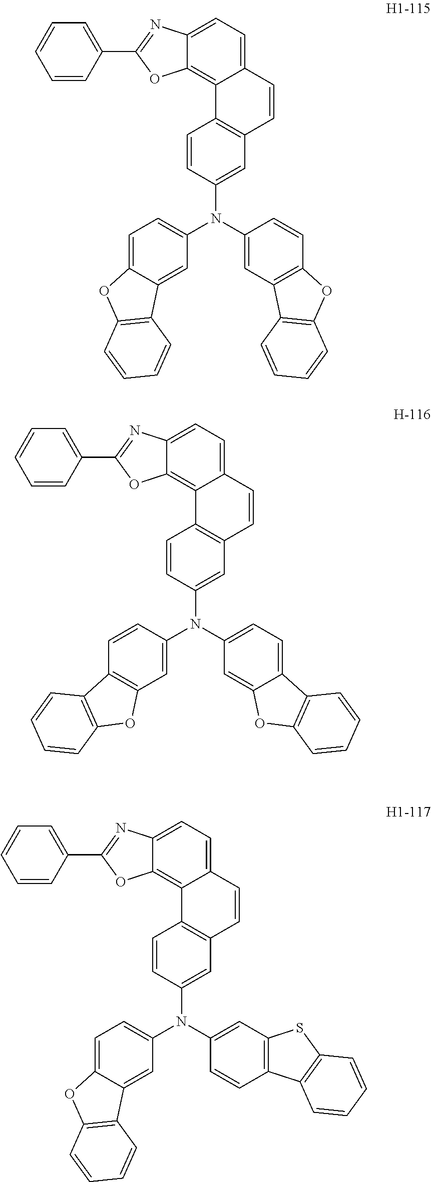 [SMILES: c1ccc(-c2nc3ccc4ccc5cc(N(c6ccc7oc8ccccc8c7c6)c6ccc7oc8ccccc8c7c6)ccc5c4c3o2)cc1 ; c1ccc(-c2nc3ccc4ccc5cc(N(c6ccc7c(c6)oc6ccccc67)c6ccc7c(c6)oc6ccccc67)ccc5c4c3o2)cc1 ; c1ccc(-c2nc3ccc4ccc5cc(N(c6ccc7c(c6)sc6ccccc67)c6ccc7oc8ccccc8c7c6)ccc5c4c3o2)cc1]

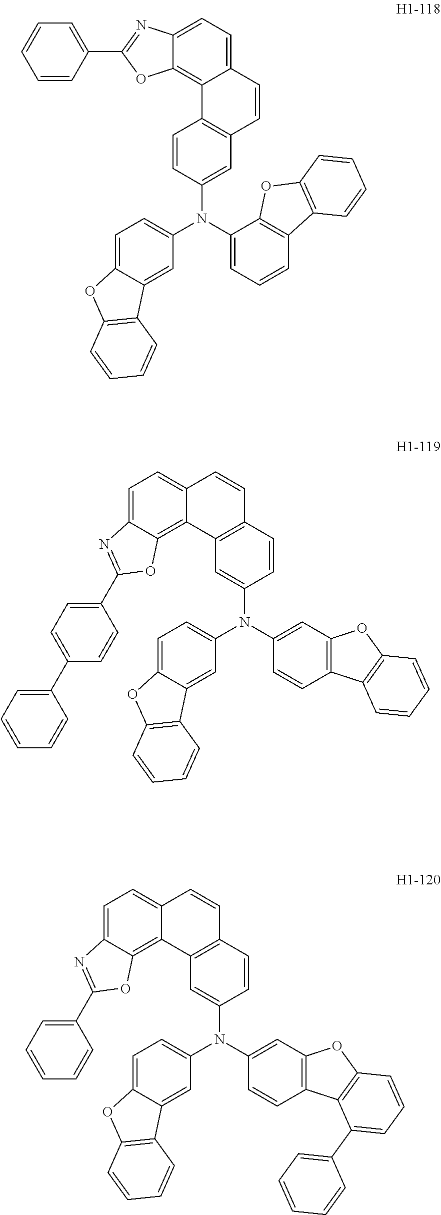 [SMILES: c1ccc(-c2nc3ccc4ccc5cc(N(c6ccc7oc8ccccc8c7c6)c6cccc7c6oc6ccccc67)ccc5c4c3o2)cc1 ; c1ccc(-c2ccc(-c3nc4ccc5ccc6ccc(N(c7ccc8c(c7)oc7ccccc78)c7ccc8oc9ccccc9c8c7)cc6c5c4o3)cc2)cc1 ; c1ccc(-c2nc3ccc4ccc5ccc(N(c6ccc7c(c6)oc6cccc(-c8ccccc8)c67)c6ccc7oc8ccccc8c7c6)cc5c4c3o2)cc1]

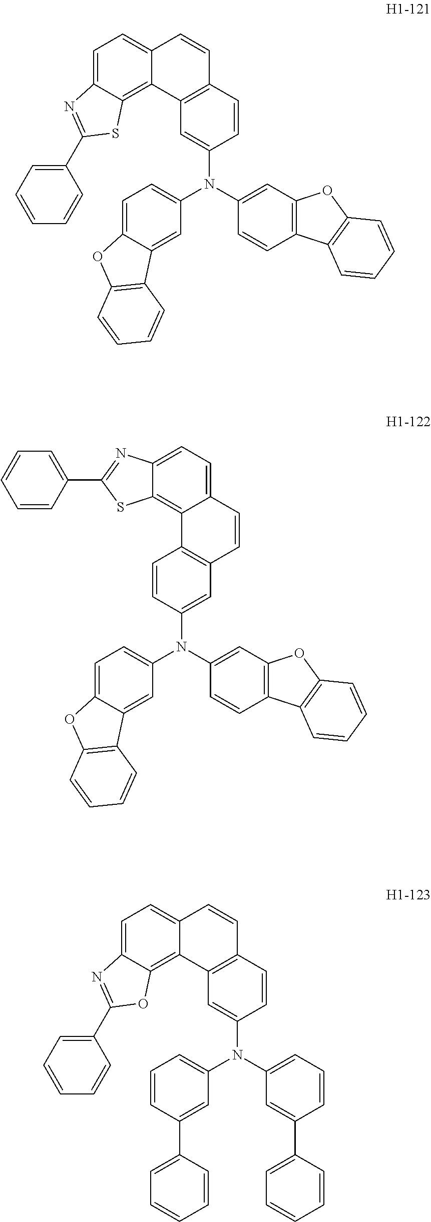 [SMILES: c1ccc(-c2nc3ccc4ccc5ccc(N(c6ccc7c(c6)oc6ccccc67)c6ccc7oc8ccccc8c7c6)cc5c4c3s2)cc1 ; c1ccc(-c2nc3ccc4ccc5cc(N(c6ccc7c(c6)oc6ccccc67)c6ccc7oc8ccccc8c7c6)ccc5c4c3s2)cc1 ; c1ccc(-c2cccc(N(c3cccc(-c4ccccc4)c3)c3ccc4ccc5ccc6nc(-c7ccccc7)oc6c5c4c3)c2)cc1]

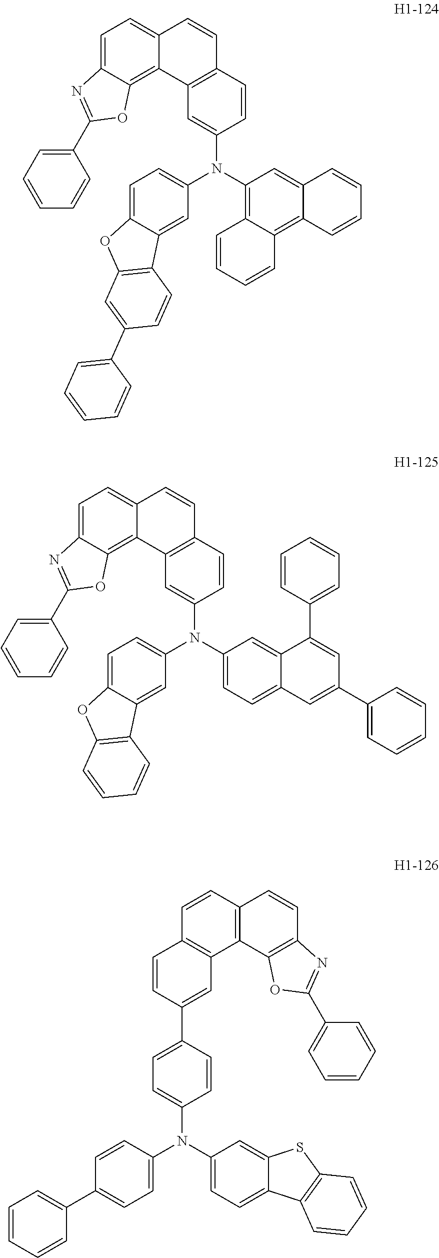 [SMILES: c1ccc(-c2ccc3c(c2)oc2ccc(N(c4ccc5ccc6ccc7nc(-c8ccccc8)oc7c6c5c4)c4cc5ccccc5c5ccccc45)cc23)cc1 ; c1ccc(-c2cc(-c3ccccc3)c3cc(N(c4ccc5oc6ccccc6c5c4)c4ccc5ccc6ccc7nc(-c8ccccc8)oc7c6c5c4)ccc3c2)cc1 ; c1ccc(-c2ccc(N(c3ccc(-c4ccc5ccc6ccc7nc(-c8ccccc8)oc7c6c5c4)cc3)c3ccc4c(c3)sc3ccccc34)cc2)cc1]

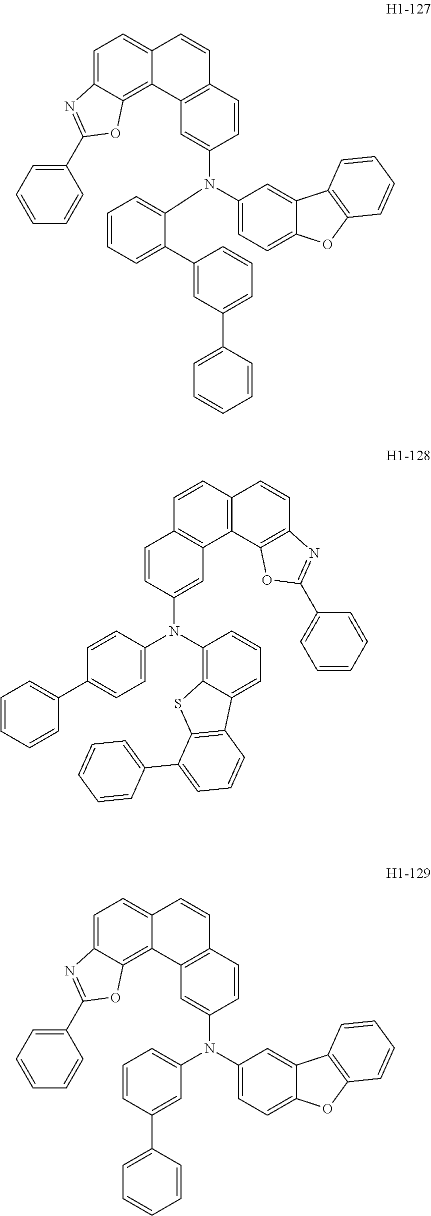 [SMILES: c1ccc(-c2cccc(-c3ccccc3N(c3ccc4oc5ccccc5c4c3)c3ccc4ccc5ccc6nc(-c7ccccc7)oc6c5c4c3)c2)cc1 ; c1ccc(-c2ccc(N(c3ccc4ccc5ccc6nc(-c7ccccc7)oc6c5c4c3)c3cccc4c3sc3c(-c5ccccc5)cccc34)cc2)cc1 ; c1ccc(-c2cccc(N(c3ccc4oc5ccccc5c4c3)c3ccc4ccc5ccc6nc(-c7ccccc7)oc6c5c4c3)c2)cc1]

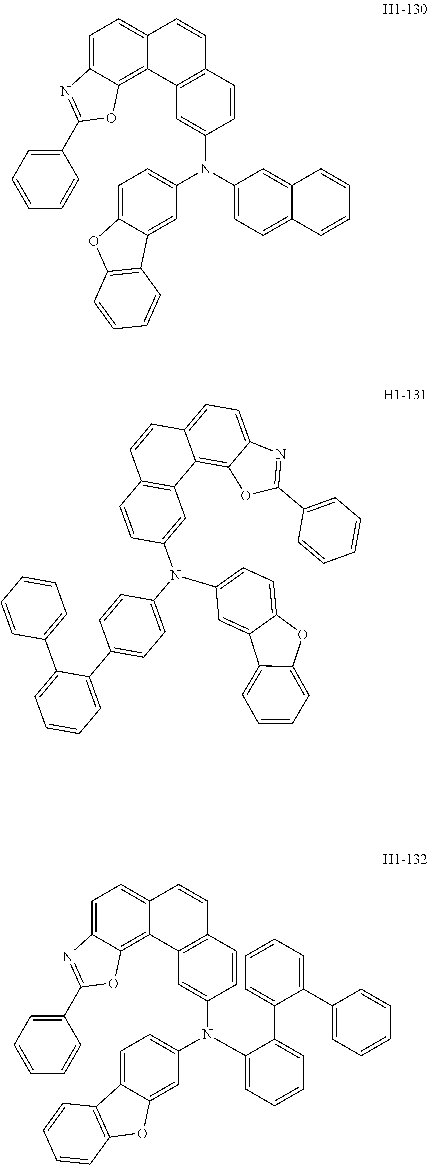 [SMILES: c1ccc(-c2nc3ccc4ccc5ccc(N(c6ccc7ccccc7c6)c6ccc7oc8ccccc8c7c6)cc5c4c3o2)cc1 ; c1ccc(-c2nc3ccc4ccc5ccc(N(c6ccc(-c7ccccc7-c7ccccc7)cc6)c6ccc7oc8ccccc8c7c6)cc5c4c3o2)cc1 ; c1ccc(-c2nc3ccc4ccc5ccc(N(c6ccc7c(c6)oc6ccccc67)c6ccccc6-c6ccccc6-c6ccccc6)cc5c4c3o2)cc1]

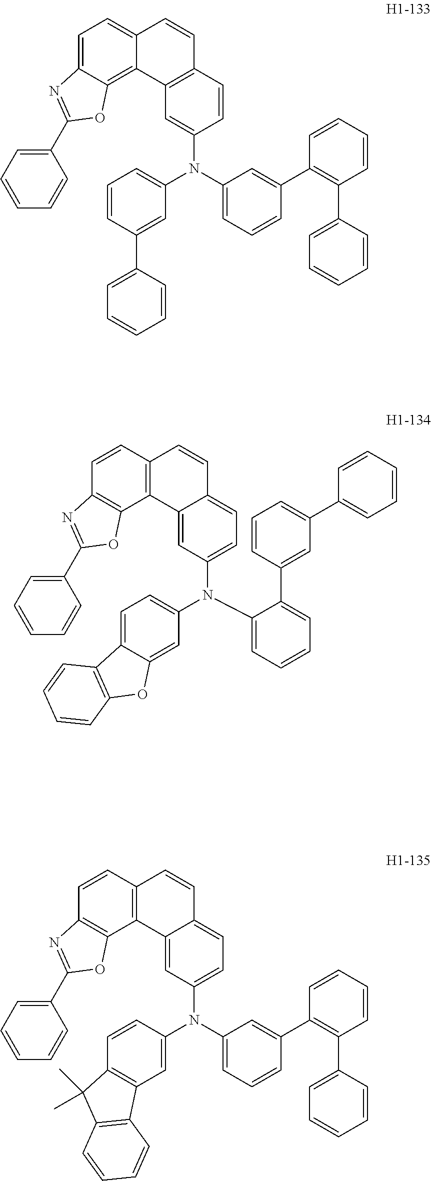 [SMILES: c1ccc(-c2cccc(N(c3cccc(-c4ccccc4-c4ccccc4)c3)c3ccc4ccc5ccc6nc(-c7ccccc7)oc6c5c4c3)c2)cc1 ; c1ccc(-c2cccc(-c3ccccc3N(c3ccc4c(c3)oc3ccccc34)c3ccc4ccc5ccc6nc(-c7ccccc7)oc6c5c4c3)c2)cc1 ; CC1(C)c2ccccc2-c2cc(N(c3cccc(-c4ccccc4-c4ccccc4)c3)c3ccc4ccc5ccc6nc(-c7ccccc7)oc6c5c4c3)ccc21]

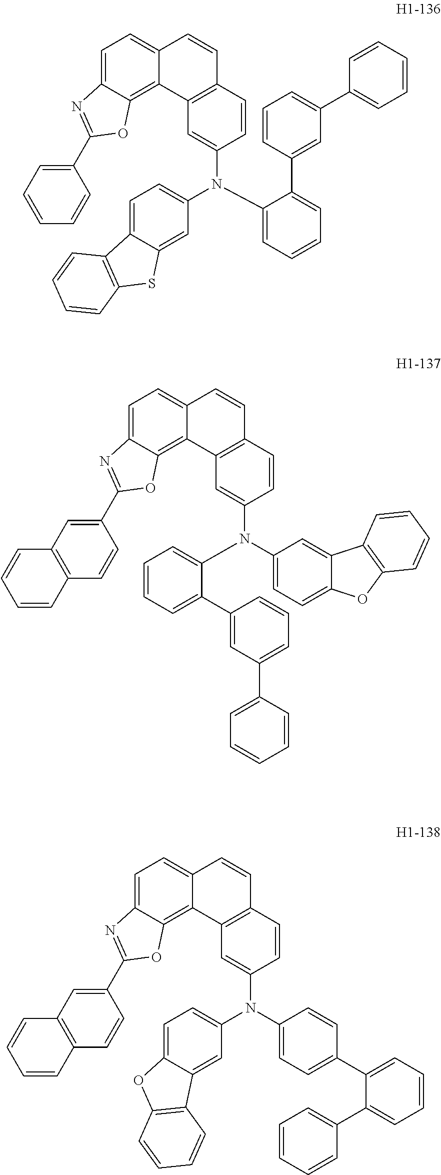 [SMILES: c1ccc(-c2cccc(-c3ccccc3N(c3ccc4c(c3)sc3ccccc34)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccc8ccccc8c7)oc6c5c4c3)c2)cc1 ; c1ccc(-c2ccccc2-c2ccc(N(c3ccc4oc5ccccc5c4c3)c3ccc4ccc5ccc6nc(-c7ccc8ccccc8c7)oc6c5c4c3)cc2)cc1]

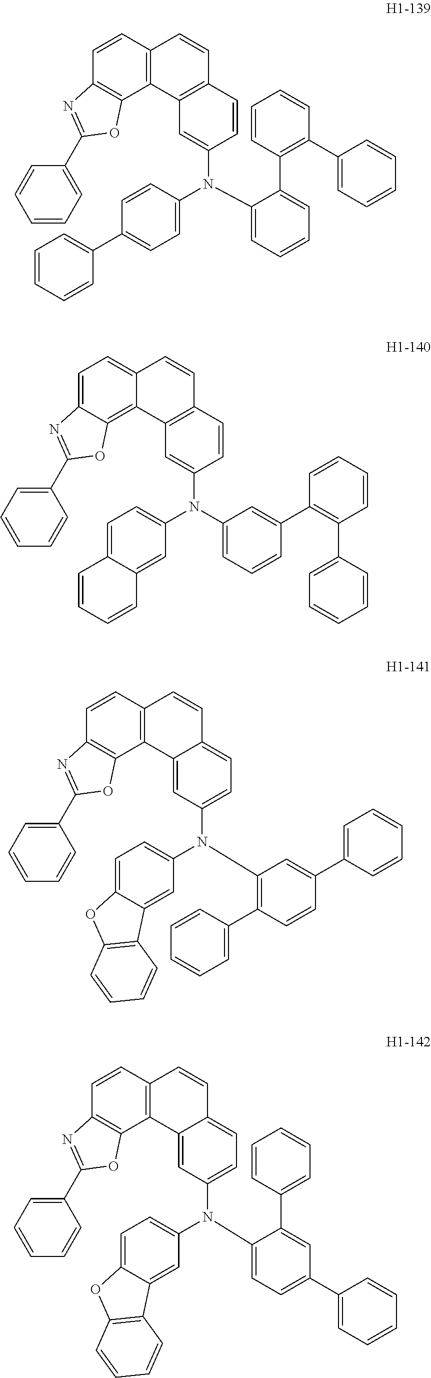 [SMILES: c1ccc(-c2ccc(N(c3ccc4ccc5ccc6nc(-c7ccccc7)oc6c5c4c3)c3ccccc3-c3ccccc3-c3ccccc3)cc2)cc1 ; c1ccc(-c2nc3ccc4ccc5ccc(N(c6cccc(-c7ccccc7-c7ccccc7)c6)c6ccc7ccccc7c6)cc5c4c3o2)cc1 ; c1ccc(-c2ccc(-c3ccccc3)c(N(c3ccc4oc5ccccc5c4c3)c3ccc4ccc5ccc6nc(-c7ccccc7)oc6c5c4c3)c2)cc1 ; c1ccc(-c2ccc(N(c3ccc4oc5ccccc5c4c3)c3ccc4ccc5ccc6nc(-c7ccccc7)oc6c5c4c3)c(-c3ccccc3)c2)cc1]

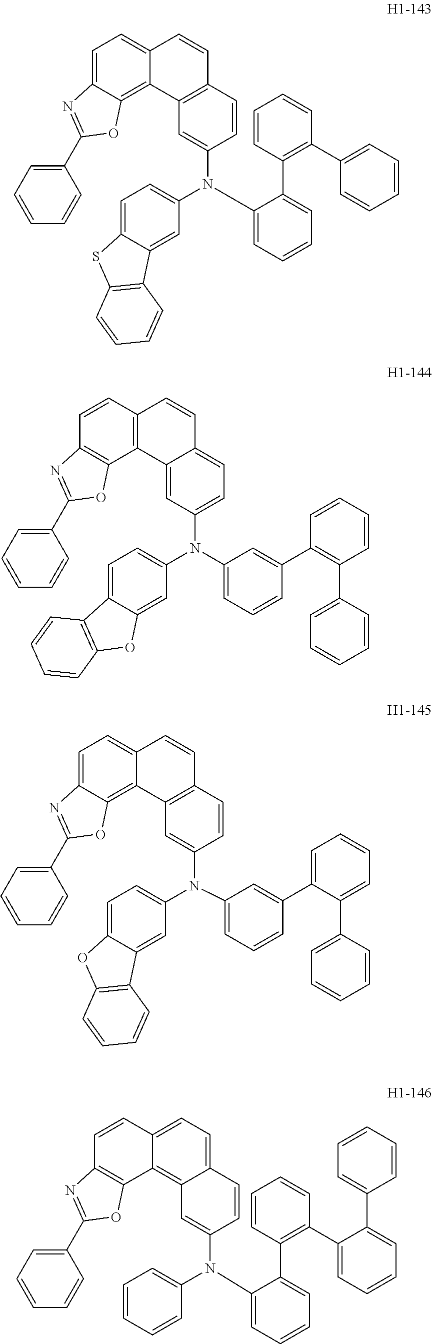 [SMILES: c1ccc(-c2nc3ccc4ccc5ccc(N(c6ccc7sc8ccccc8c7c6)c6ccccc6-c6ccccc6-c6ccccc6)cc5c4c3o2)cc1 ; c1ccc(-c2nc3ccc4ccc5ccc(N(c6cccc(-c7ccccc7-c7ccccc7)c6)c6ccc7c(c6)oc6ccccc67)cc5c4c3o2)cc1 ; c1ccc(-c2nc3ccc4ccc5ccc(N(c6cccc(-c7ccccc7-c7ccccc7)c6)c6ccc7oc8ccccc8c7c6)cc5c4c3o2)cc1 ; c1ccc(-c2nc3ccc4ccc5ccc(N(c6ccccc6)c6ccccc6-c6ccccc6-c6ccccc6-c6ccccc6)cc5c4c3o2)cc1]

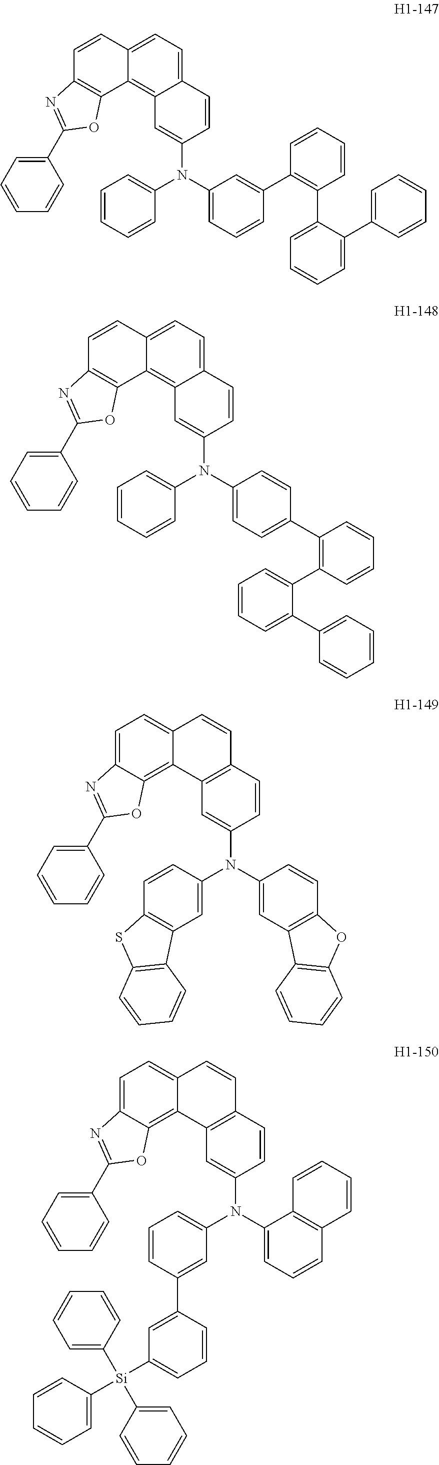

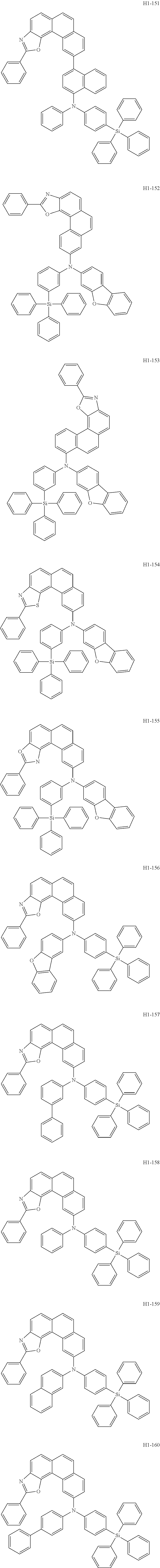

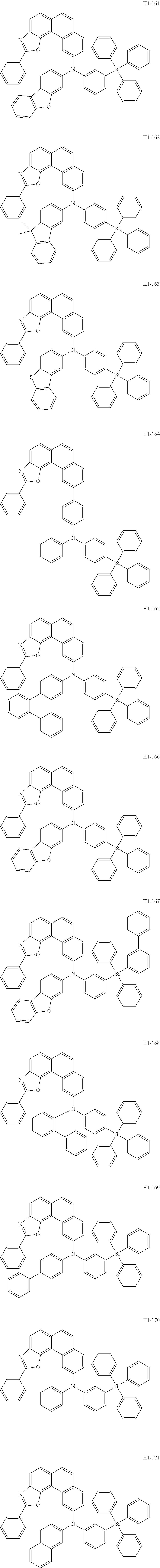

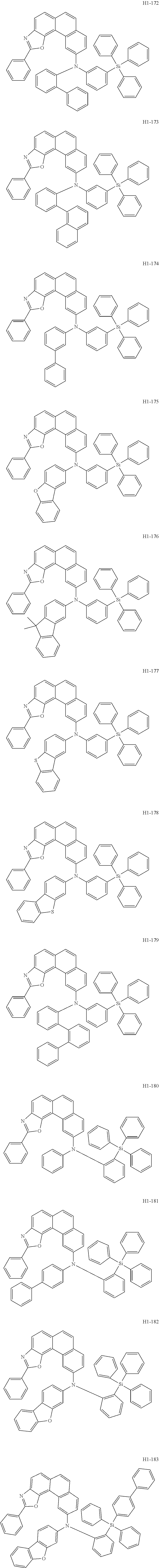

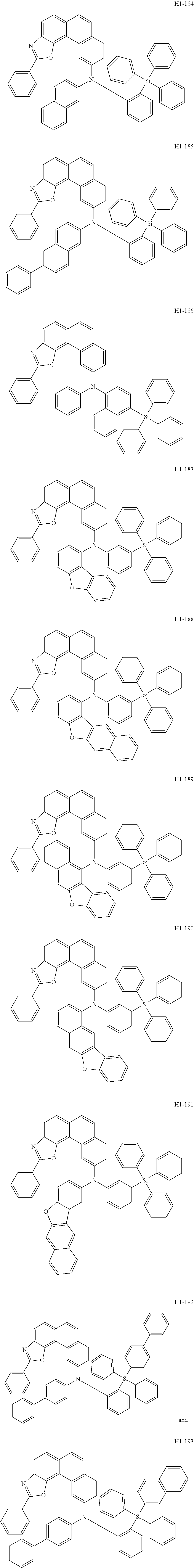

8. The plurality of host materials according to claim 1, wherein the compound represented by Formula 2 is selected from the following compounds:

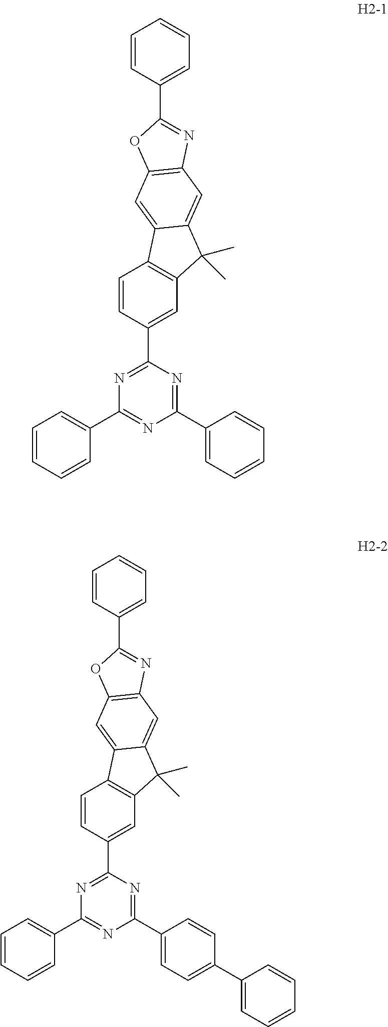 [SMILES: CC1(C)c2cc(-c3nc(-c4ccccc4)nc(-c4ccccc4)n3)ccc2-c2cc3oc(-c4ccccc4)nc3cc21 ; CC1(C)c2cc(-c3nc(-c4ccccc4)nc(-c4ccc(-c5ccccc5)cc4)n3)ccc2-c2cc3oc(-c4ccccc4)nc3cc21]

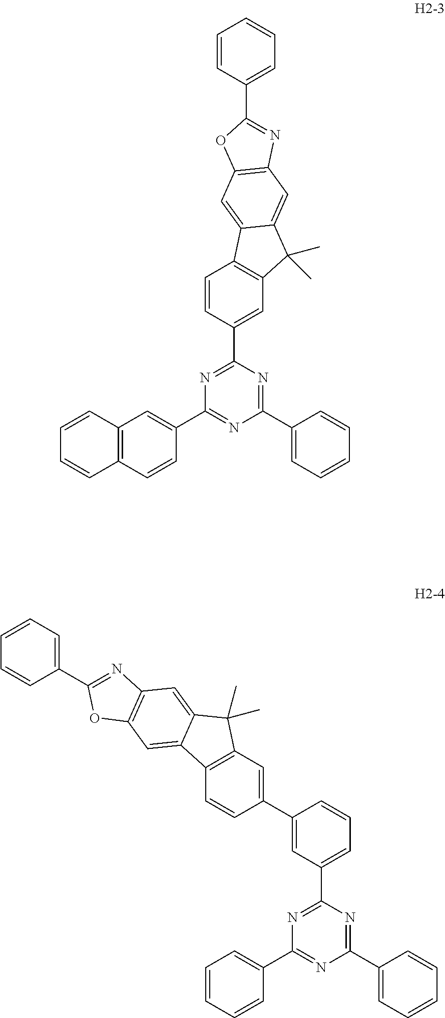 [SMILES: CC1(C)c2cc(-c3nc(-c4ccccc4)nc(-c4ccc5ccccc5c4)n3)ccc2-c2cc3oc(-c4ccccc4)nc3cc21 ; CC1(C)c2cc(-c3cccc(-c4nc(-c5ccccc5)nc(-c5ccccc5)n4)c3)ccc2-c2cc3oc(-c4ccccc4)nc3cc21]

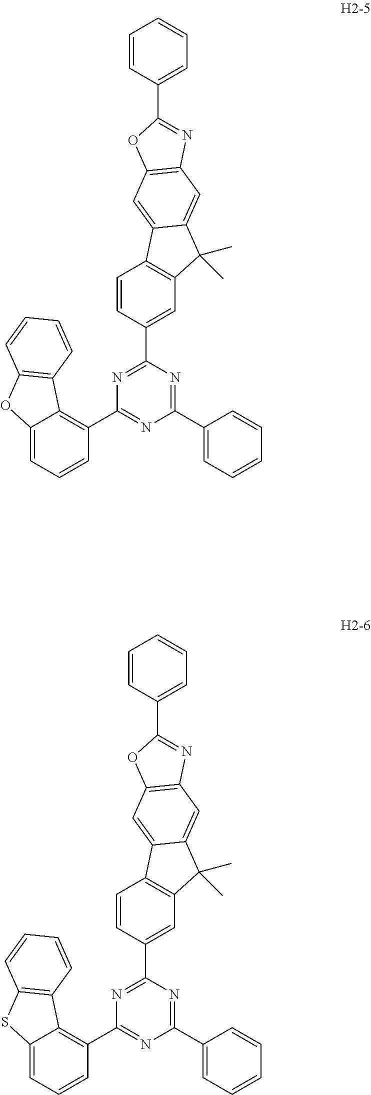 [SMILES: CC1(C)c2cc(-c3nc(-c4ccccc4)nc(-c4cccc5oc6ccccc6c45)n3)ccc2-c2cc3oc(-c4ccccc4)nc3cc21 ; CC1(C)c2cc(-c3nc(-c4ccccc4)nc(-c4cccc5sc6ccccc6c45)n3)ccc2-c2cc3oc(-c4ccccc4)nc3cc21]

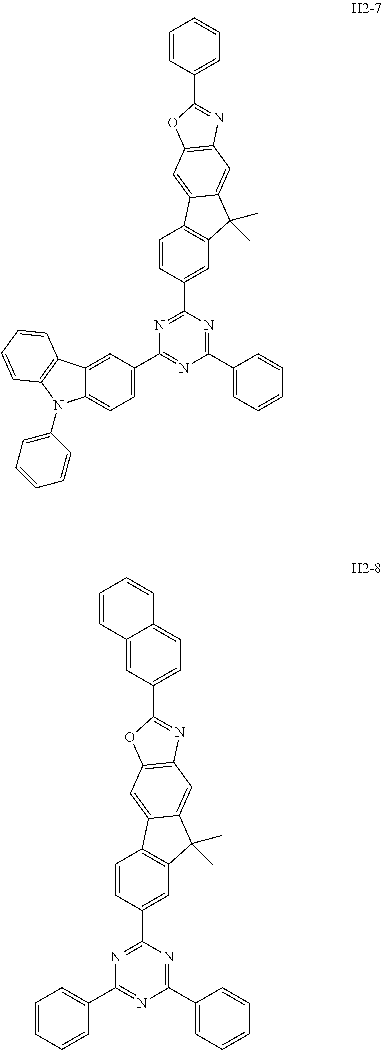 [SMILES: CC1(C)c2cc(-c3nc(-c4ccccc4)nc(-c4ccc5c(c4)c4ccccc4n5-c4ccccc4)n3)ccc2-c2cc3oc(-c4ccccc4)nc3cc21 ; CC1(C)c2cc(-c3nc(-c4ccccc4)nc(-c4ccccc4)n3)ccc2-c2cc3oc(-c4ccc5ccccc5c4)nc3cc21]

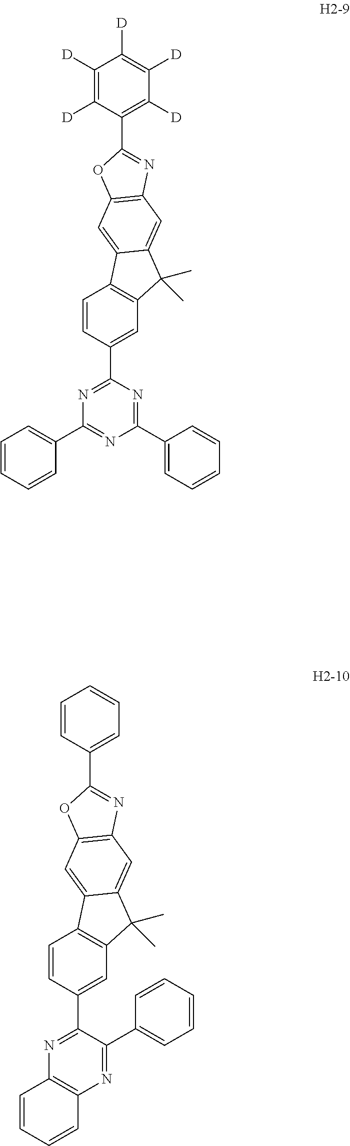 [SMILES: [2H]c1c([2H])c([2H])c(-c2nc3cc4c(cc3o2)-c2ccc(-c3nc(-c5ccccc5)nc(-c5ccccc5)n3)cc2C4(C)C)c([2H])c1[2H] ; CC1(C)c2cc(-c3nc4ccccc4nc3-c3ccccc3)ccc2-c2cc3oc(-c4ccccc4)nc3cc21]

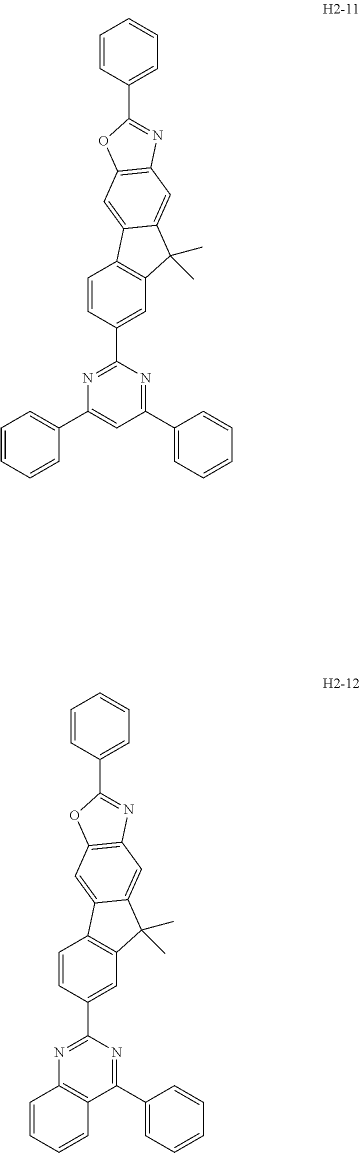 [SMILES: CC1(C)c2cc(-c3nc(-c4ccccc4)cc(-c4ccccc4)n3)ccc2-c2cc3oc(-c4ccccc4)nc3cc21 ; CC1(C)c2cc(-c3nc(-c4ccccc4)c4ccccc4n3)ccc2-c2cc3oc(-c4ccccc4)nc3cc21]

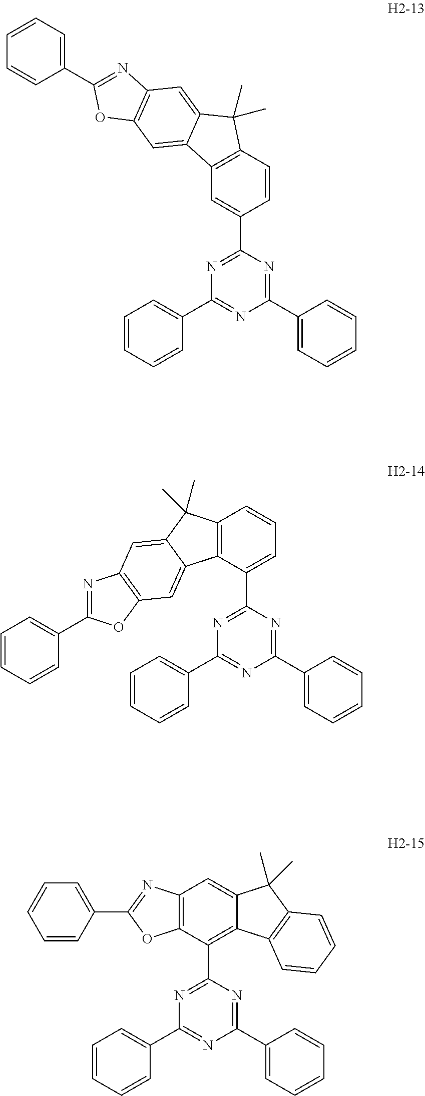 [SMILES: CC1(C)c2ccc(-c3nc(-c4ccccc4)nc(-c4ccccc4)n3)cc2-c2cc3oc(-c4ccccc4)nc3cc21 ; CC1(C)c2cc3nc(-c4ccccc4)oc3cc2-c2c(-c3nc(-c4ccccc4)nc(-c4ccccc4)n3)cccc21 ; CC1(C)c2ccccc2-c2c1cc1nc(-c3ccccc3)oc1c2-c1nc(-c2ccccc2)nc(-c2ccccc2)n1]

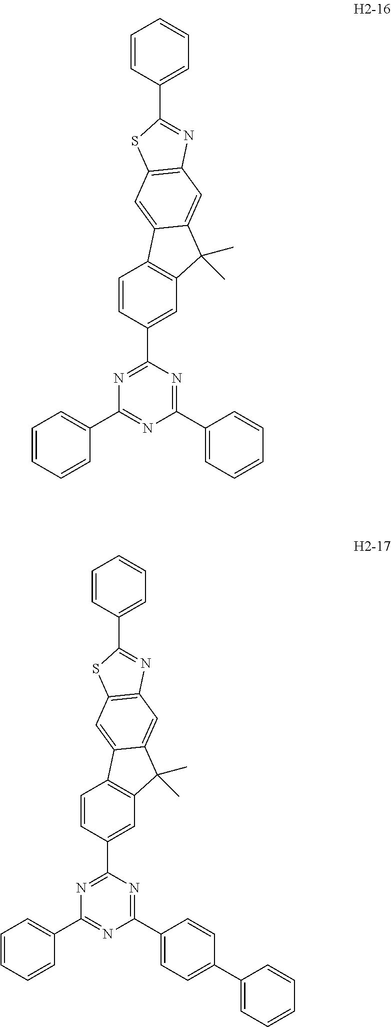 [SMILES: CC1(C)c2cc(-c3nc(-c4ccccc4)nc(-c4ccccc4)n3)ccc2-c2cc3sc(-c4ccccc4)nc3cc21 ; CC1(C)c2cc(-c3nc(-c4ccccc4)nc(-c4ccc(-c5ccccc5)cc4)n3)ccc2-c2cc3sc(-c4ccccc4)nc3cc21]

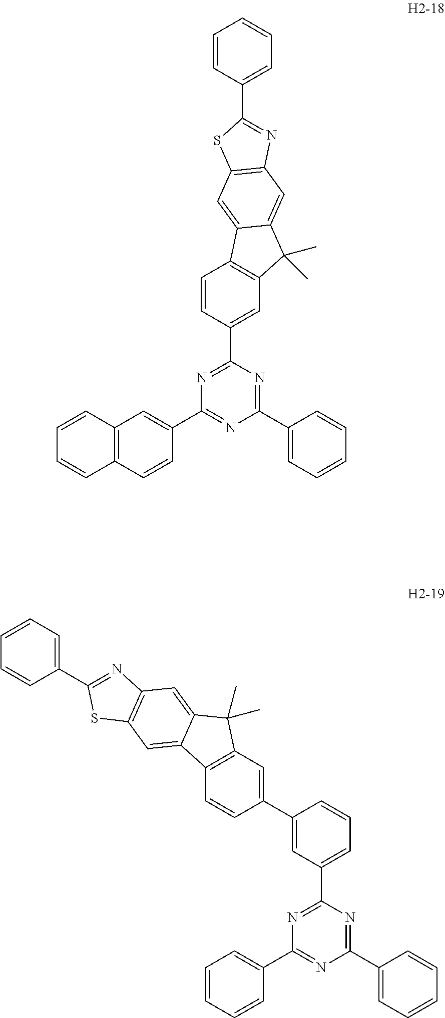 [SMILES: CC1(C)c2cc(-c3nc(-c4ccccc4)nc(-c4ccc5ccccc5c4)n3)ccc2-c2cc3sc(-c4ccccc4)nc3cc21 ; CC1(C)c2cc(-c3cccc(-c4nc(-c5ccccc5)nc(-c5ccccc5)n4)c3)ccc2-c2cc3sc(-c4ccccc4)nc3cc21]

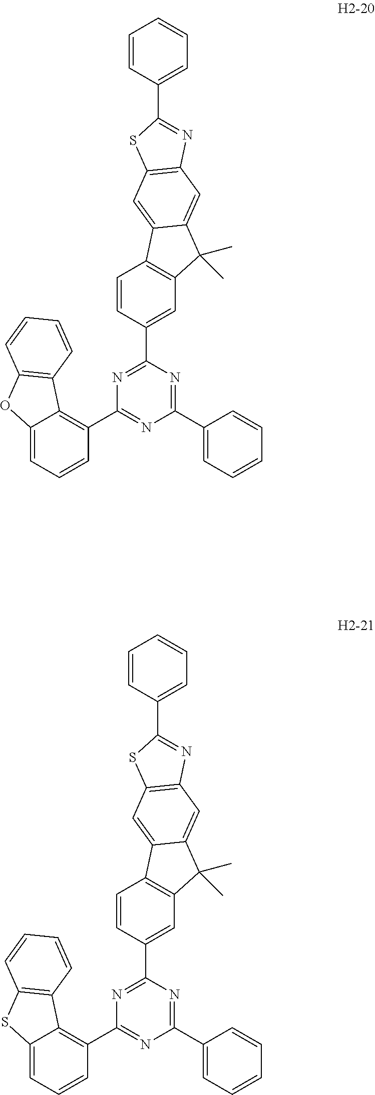 [SMILES: CC1(C)c2cc(-c3nc(-c4ccccc4)nc(-c4cccc5oc6ccccc6c45)n3)ccc2-c2cc3sc(-c4ccccc4)nc3cc21 ; CC1(C)c2cc(-c3nc(-c4ccccc4)nc(-c4cccc5sc6ccccc6c45)n3)ccc2-c2cc3sc(-c4ccccc4)nc3cc21]

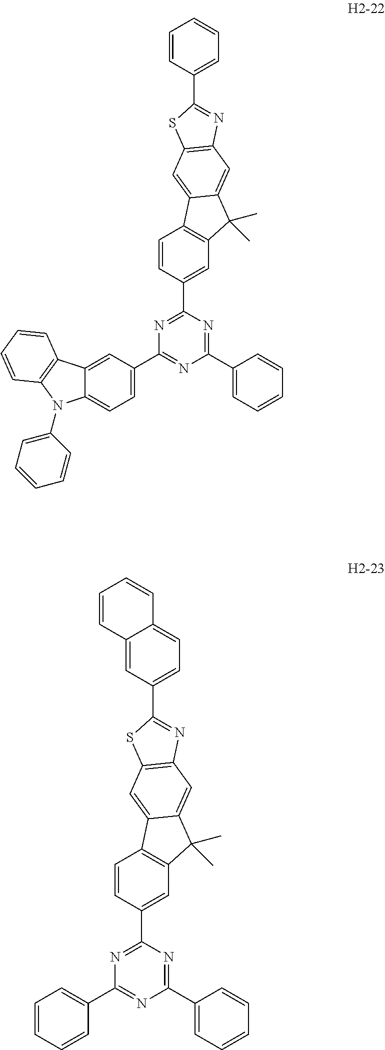 [SMILES: CC1(C)c2cc(-c3nc(-c4ccccc4)nc(-c4ccc5c(c4)c4ccccc4n5-c4ccccc4)n3)ccc2-c2cc3sc(-c4ccccc4)nc3cc21 ; CC1(C)c2cc(-c3nc(-c4ccccc4)nc(-c4ccccc4)n3)ccc2-c2cc3sc(-c4ccc5ccccc5c4)nc3cc21]

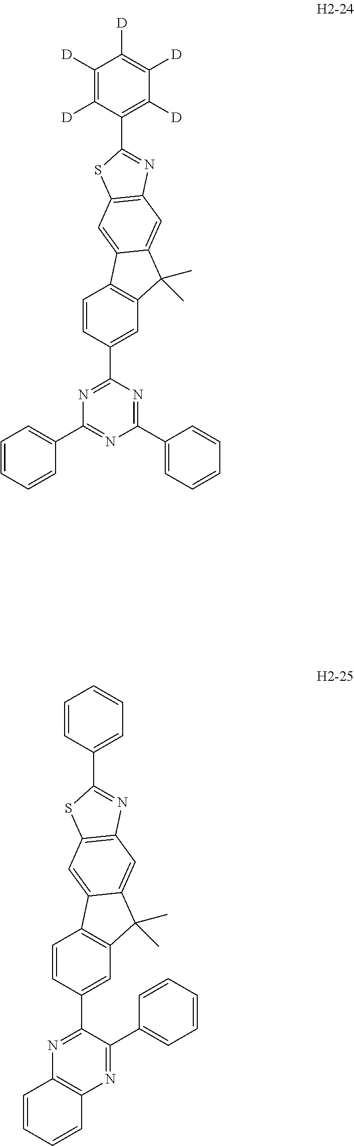 [SMILES: [2H]c1c([2H])c([2H])c(-c2nc3cc4c(cc3s2)-c2ccc(-c3nc(-c5ccccc5)nc(-c5ccccc5)n3)cc2C4(C)C)c([2H])c1[2H] ; CC1(C)c2cc(-c3nc4ccccc4nc3-c3ccccc3)ccc2-c2cc3sc(-c4ccccc4)nc3cc21]

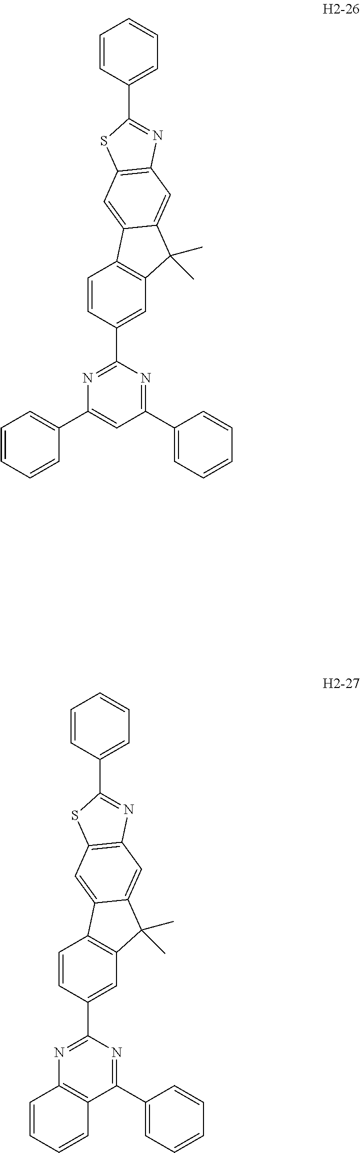 [SMILES: CC1(C)c2cc(-c3nc(-c4ccccc4)cc(-c4ccccc4)n3)ccc2-c2cc3sc(-c4ccccc4)nc3cc21 ; CC1(C)c2cc(-c3nc(-c4ccccc4)c4ccccc4n3)ccc2-c2cc3sc(-c4ccccc4)nc3cc21]

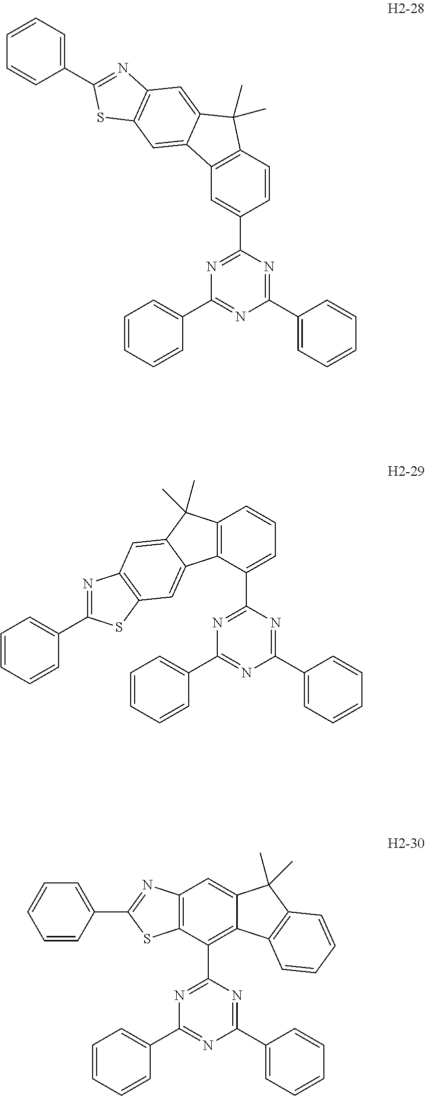 [SMILES: CC1(C)c2ccc(-c3nc(-c4ccccc4)nc(-c4ccccc4)n3)cc2-c2cc3sc(-c4ccccc4)nc3cc21 ; CC1(C)c2cc3nc(-c4ccccc4)sc3cc2-c2c(-c3nc(-c4ccccc4)nc(-c4ccccc4)n3)cccc21 ; CC1(C)c2ccccc2-c2c1cc1nc(-c3ccccc3)sc1c2-c1nc(-c2ccccc2)nc(-c2ccccc2)n1]

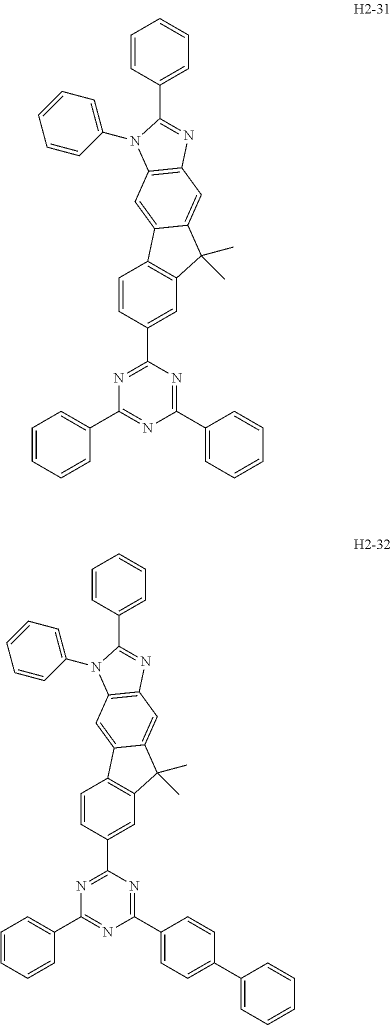 [SMILES: CC1(C)c2cc(-c3nc(-c4ccccc4)nc(-c4ccccc4)n3)ccc2-c2cc3c(cc21)nc(-c1ccccc1)n3-c1ccccc1 ; CC1(C)c2cc(-c3nc(-c4ccccc4)nc(-c4ccc(-c5ccccc5)cc4)n3)ccc2-c2cc3c(cc21)nc(-c1ccccc1)n3-c1ccccc1]

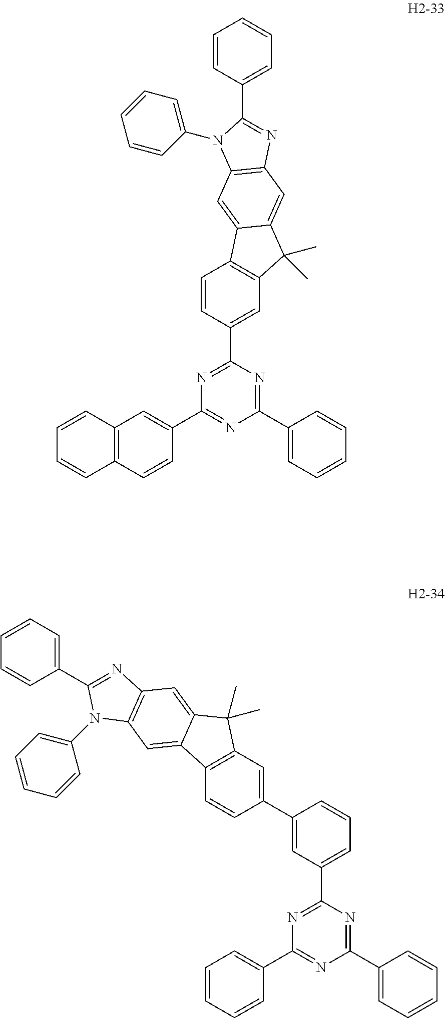 [SMILES: CC1(C)c2cc(-c3nc(-c4ccccc4)nc(-c4ccc5ccccc5c4)n3)ccc2-c2cc3c(cc21)nc(-c1ccccc1)n3-c1ccccc1 ; CC1(C)c2cc(-c3cccc(-c4nc(-c5ccccc5)nc(-c5ccccc5)n4)c3)ccc2-c2cc3c(cc21)nc(-c1ccccc1)n3-c1ccccc1]

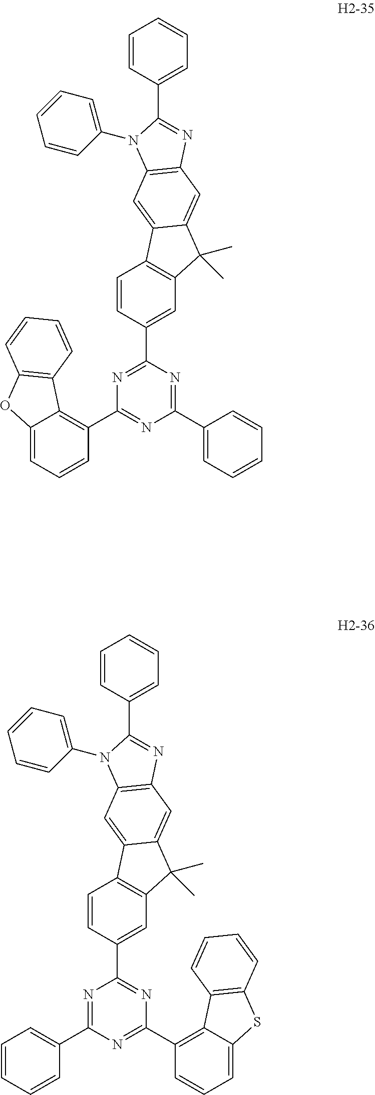 [SMILES: CC1(C)c2cc(-c3nc(-c4ccccc4)nc(-c4cccc5oc6ccccc6c45)n3)ccc2-c2cc3c(cc21)nc(-c1ccccc1)n3-c1ccccc1 ; CC1(C)c2cc(-c3nc(-c4ccccc4)nc(-c4cccc5sc6ccccc6c45)n3)ccc2-c2cc3c(cc21)nc(-c1ccccc1)n3-c1ccccc1]

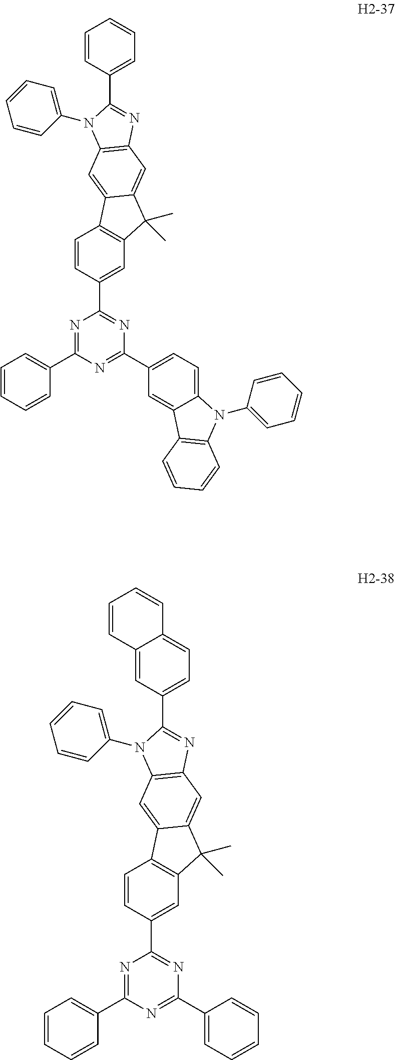 [SMILES: CC1(C)c2cc(-c3nc(-c4ccccc4)nc(-c4ccc5c(c4)c4ccccc4n5-c4ccccc4)n3)ccc2-c2cc3c(cc21)nc(-c1ccccc1)n3-c1ccccc1 ; CC1(C)c2cc(-c3nc(-c4ccccc4)nc(-c4ccccc4)n3)ccc2-c2cc3c(cc21)nc(-c1ccc2ccccc2c1)n3-c1ccccc1]

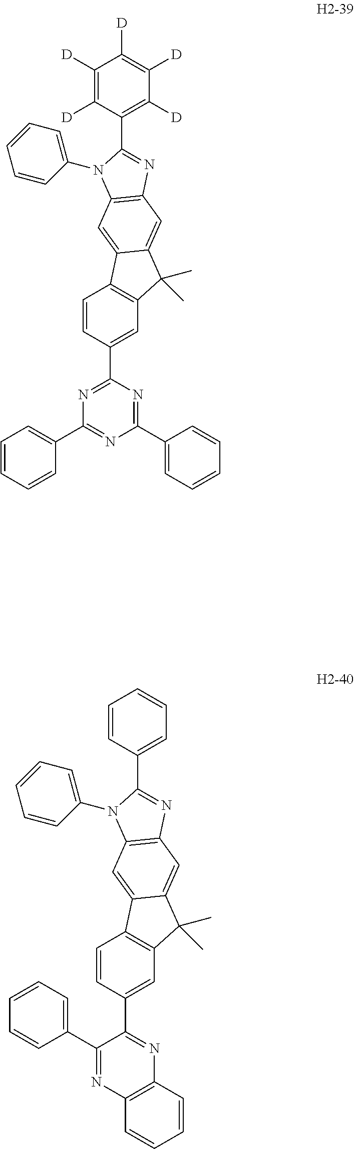 [SMILES: [2H]c1c([2H])c([2H])c(-c2nc3cc4c(cc3n2-c2ccccc2)-c2ccc(-c3nc(-c5ccccc5)nc(-c5ccccc5)n3)cc2C4(C)C)c([2H])c1[2H] ; CC1(C)c2cc(-c3nc4ccccc4nc3-c3ccccc3)ccc2-c2cc3c(cc21)nc(-c1ccccc1)n3-c1ccccc1]

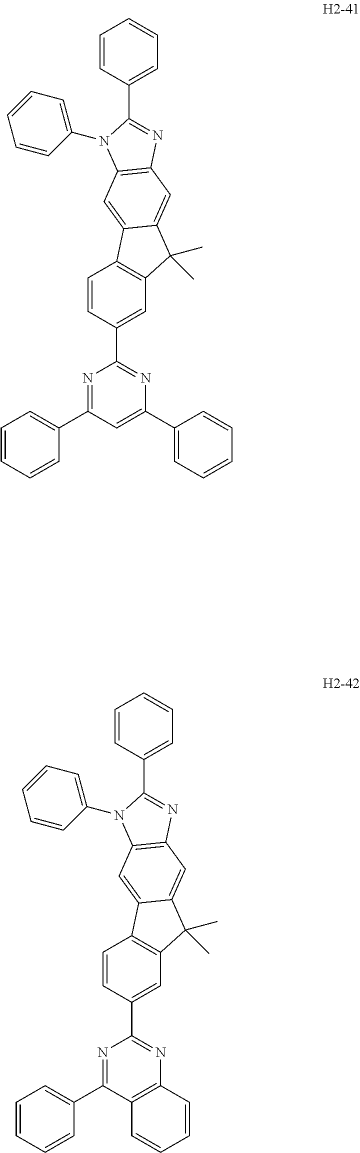 [SMILES: CC1(C)c2cc(-c3nc(-c4ccccc4)cc(-c4ccccc4)n3)ccc2-c2cc3c(cc21)nc(-c1ccccc1)n3-c1ccccc1 ; CC1(C)c2cc(-c3nc(-c4ccccc4)c4ccccc4n3)ccc2-c2cc3c(cc21)nc(-c1ccccc1)n3-c1ccccc1]

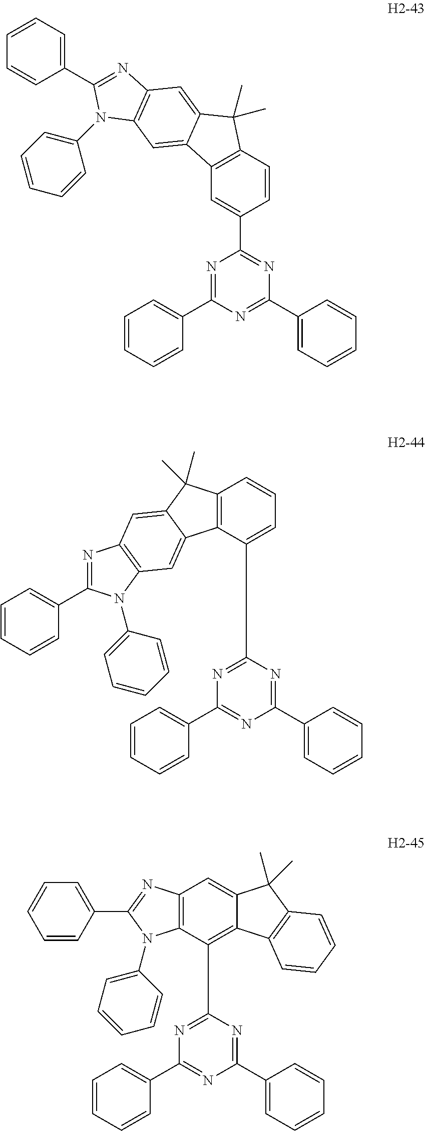 [SMILES: CC1(C)c2ccc(-c3nc(-c4ccccc4)nc(-c4ccccc4)n3)cc2-c2cc3c(cc21)nc(-c1ccccc1)n3-c1ccccc1 ; CC1(C)c2cc3nc(-c4ccccc4)n(-c4ccccc4)c3cc2-c2c(-c3nc(-c4ccccc4)nc(-c4ccccc4)n3)cccc21 ; CC1(C)c2ccccc2-c2c1cc1nc(-c3ccccc3)n(-c3ccccc3)c1c2-c1nc(-c2ccccc2)nc(-c2ccccc2)n1]

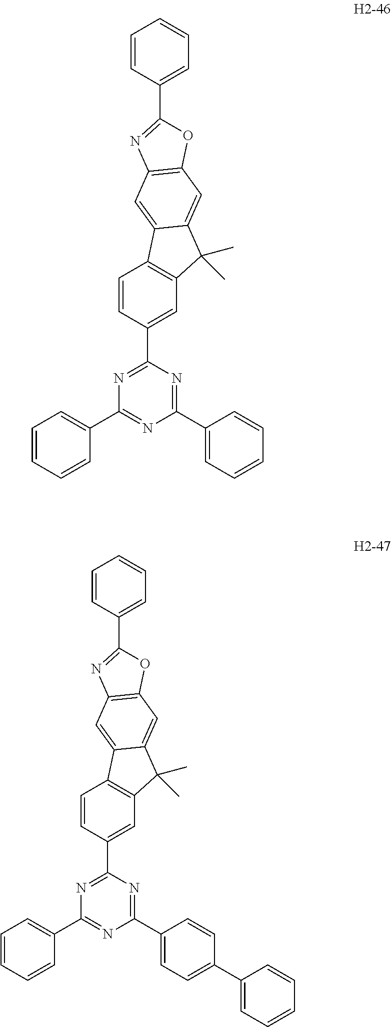 [SMILES: CC1(C)c2cc(-c3nc(-c4ccccc4)nc(-c4ccccc4)n3)ccc2-c2cc3nc(-c4ccccc4)oc3cc21 ; CC1(C)c2cc(-c3nc(-c4ccccc4)nc(-c4ccc(-c5ccccc5)cc4)n3)ccc2-c2cc3nc(-c4ccccc4)oc3cc21]

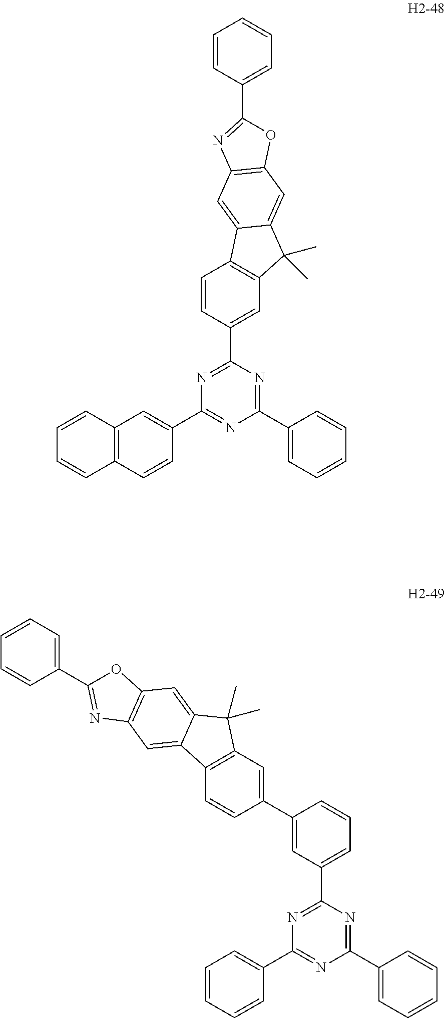 [SMILES: CC1(C)c2cc(-c3nc(-c4ccccc4)nc(-c4ccc5ccccc5c4)n3)ccc2-c2cc3nc(-c4ccccc4)oc3cc21 ; CC1(C)c2cc(-c3cccc(-c4nc(-c5ccccc5)nc(-c5ccccc5)n4)c3)ccc2-c2cc3nc(-c4ccccc4)oc3cc21]

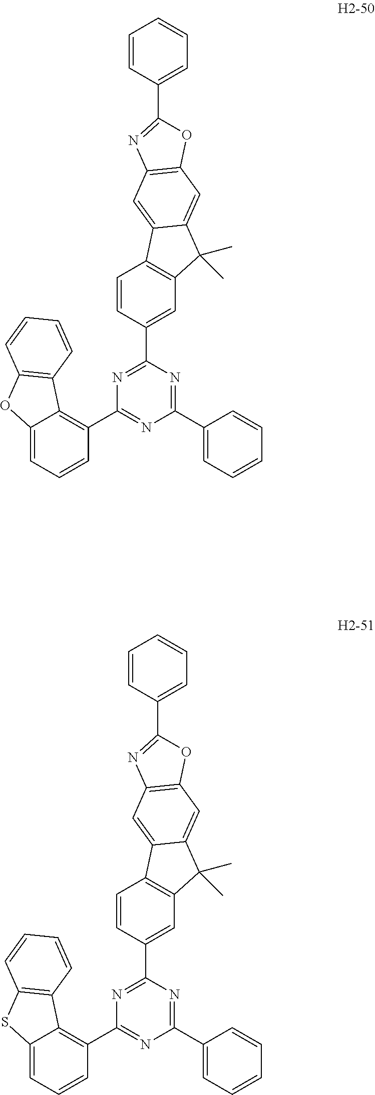 [SMILES: CC1(C)c2cc(-c3nc(-c4ccccc4)nc(-c4cccc5oc6ccccc6c45)n3)ccc2-c2cc3nc(-c4ccccc4)oc3cc21 ; CC1(C)c2cc(-c3nc(-c4ccccc4)nc(-c4cccc5sc6ccccc6c45)n3)ccc2-c2cc3nc(-c4ccccc4)oc3cc21]

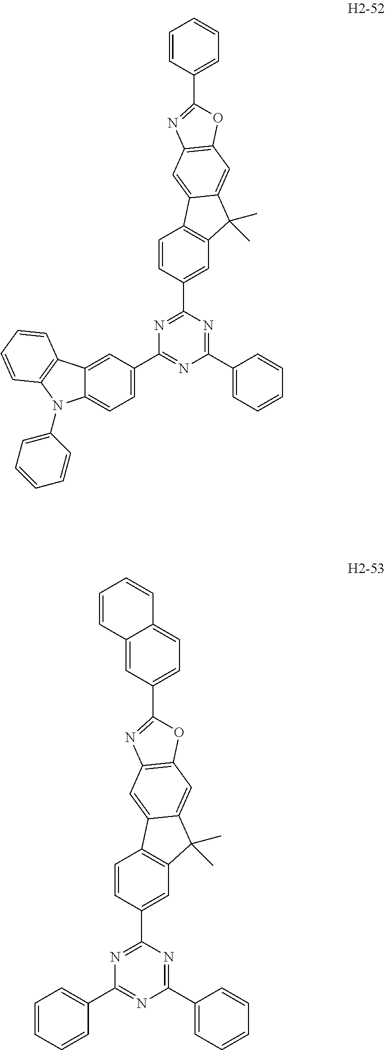 [SMILES: CC1(C)c2cc(-c3nc(-c4ccccc4)nc(-c4ccc5c(c4)c4ccccc4n5-c4ccccc4)n3)ccc2-c2cc3nc(-c4ccccc4)oc3cc21 ; CC1(C)c2cc(-c3nc(-c4ccccc4)nc(-c4ccccc4)n3)ccc2-c2cc3nc(-c4ccc5ccccc5c4)oc3cc21]

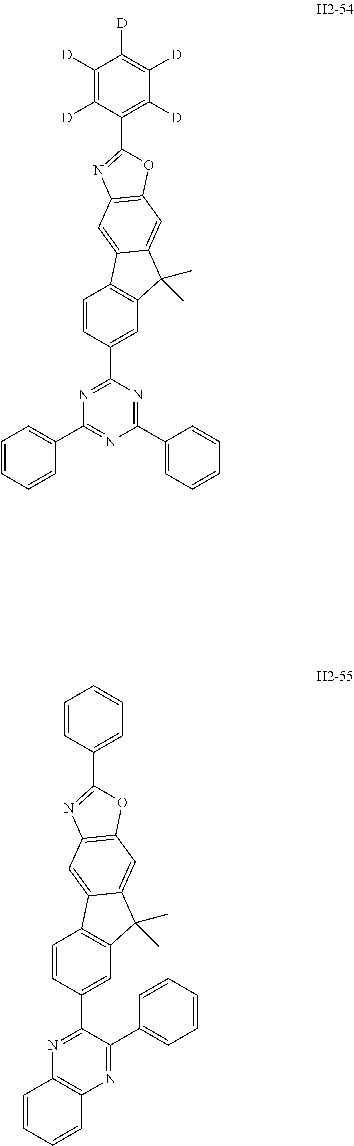 [SMILES: [2H]c1c([2H])c([2H])c(-c2nc3cc4c(cc3o2)C(C)(C)c2cc(-c3nc(-c5ccccc5)nc(-c5ccccc5)n3)ccc2-4)c([2H])c1[2H] ; CC1(C)c2cc(-c3nc4ccccc4nc3-c3ccccc3)ccc2-c2cc3nc(-c4ccccc4)oc3cc21]

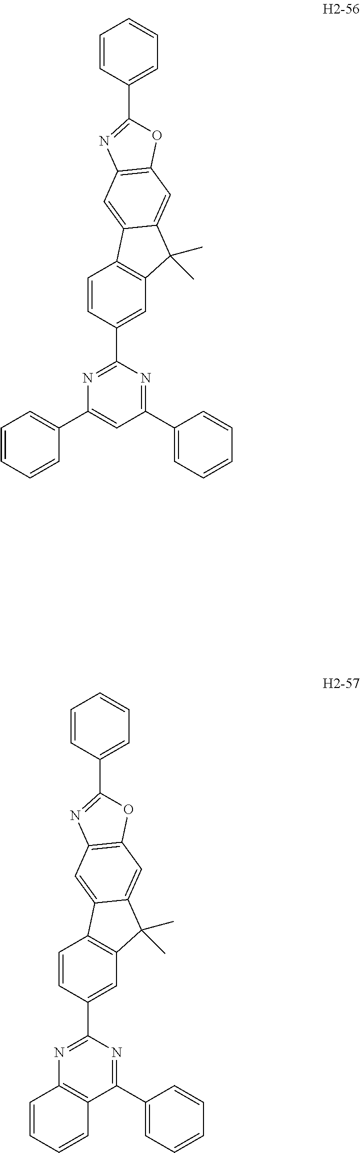 [SMILES: CC1(C)c2cc(-c3nc(-c4ccccc4)cc(-c4ccccc4)n3)ccc2-c2cc3nc(-c4ccccc4)oc3cc21 ; CC1(C)c2cc(-c3nc(-c4ccccc4)c4ccccc4n3)ccc2-c2cc3nc(-c4ccccc4)oc3cc21]

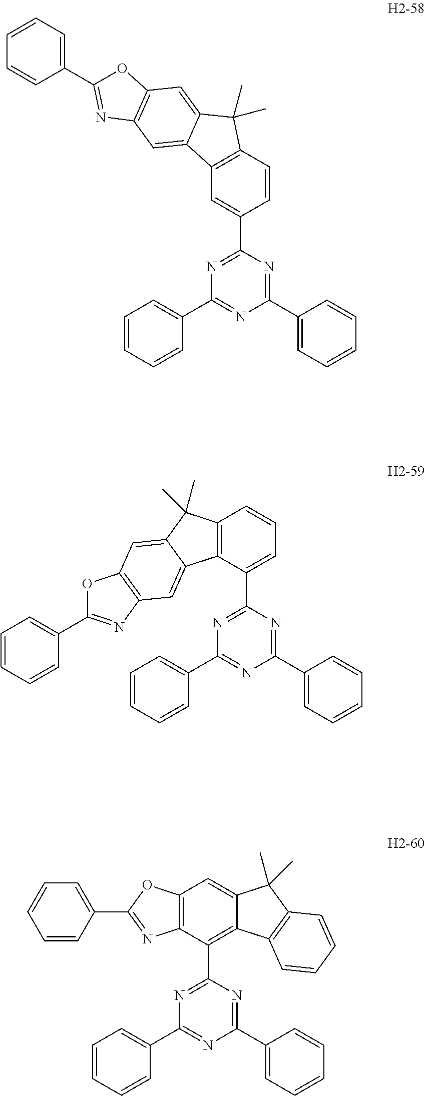 [SMILES: CC1(C)c2ccc(-c3nc(-c4ccccc4)nc(-c4ccccc4)n3)cc2-c2cc3nc(-c4ccccc4)oc3cc21 ; CC1(C)c2cc3oc(-c4ccccc4)nc3cc2-c2c(-c3nc(-c4ccccc4)nc(-c4ccccc4)n3)cccc21 ; CC1(C)c2ccccc2-c2c1cc1oc(-c3ccccc3)nc1c2-c1nc(-c2ccccc2)nc(-c2ccccc2)n1]

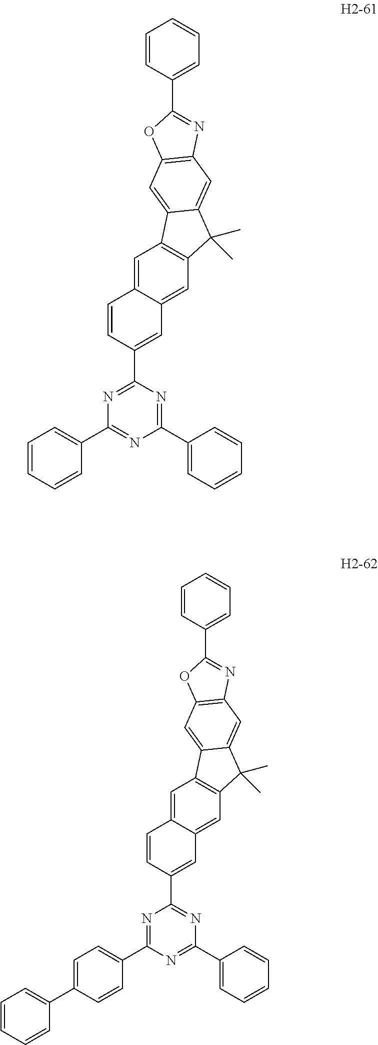 [SMILES: CC1(C)c2cc3cc(-c4nc(-c5ccccc5)nc(-c5ccccc5)n4)ccc3cc2-c2cc3oc(-c4ccccc4)nc3cc21 ; CC1(C)c2cc3cc(-c4nc(-c5ccccc5)nc(-c5ccc(-c6ccccc6)cc5)n4)ccc3cc2-c2cc3oc(-c4ccccc4)nc3cc21]

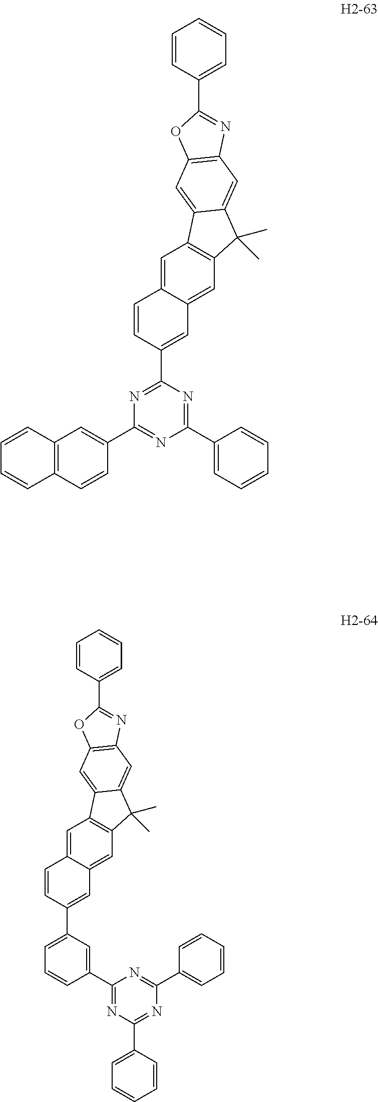 [SMILES: CC1(C)c2cc3cc(-c4nc(-c5ccccc5)nc(-c5ccc6ccccc6c5)n4)ccc3cc2-c2cc3oc(-c4ccccc4)nc3cc21 ; CC1(C)c2cc3cc(-c4cccc(-c5nc(-c6ccccc6)nc(-c6ccccc6)n5)c4)ccc3cc2-c2cc3oc(-c4ccccc4)nc3cc21]

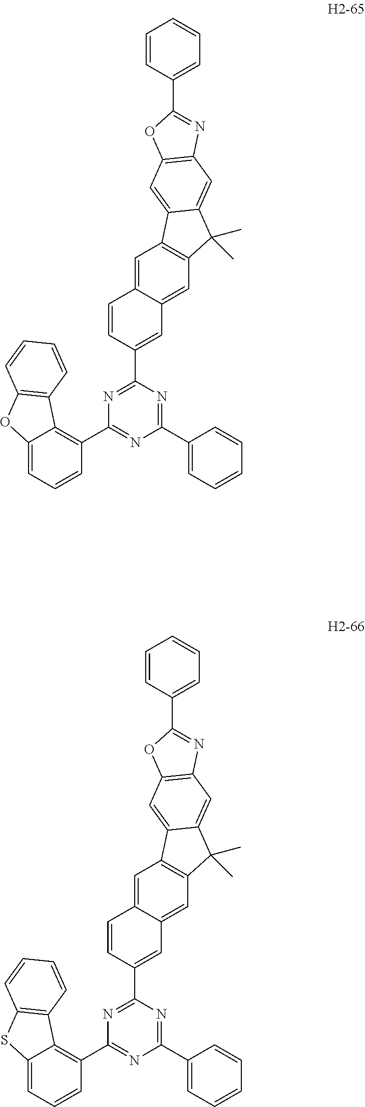 [SMILES: CC1(C)c2cc3cc(-c4nc(-c5ccccc5)nc(-c5cccc6oc7ccccc7c56)n4)ccc3cc2-c2cc3oc(-c4ccccc4)nc3cc21 ; CC1(C)c2cc3cc(-c4nc(-c5ccccc5)nc(-c5cccc6sc7ccccc7c56)n4)ccc3cc2-c2cc3oc(-c4ccccc4)nc3cc21]

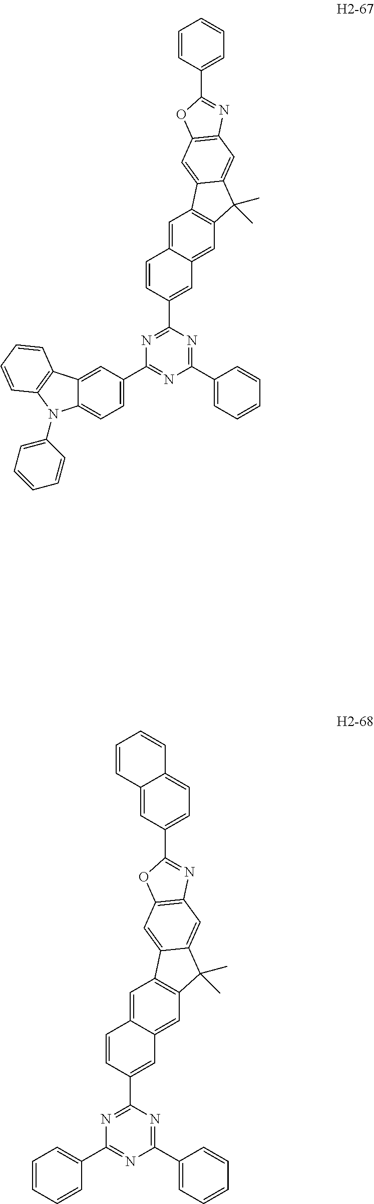 [SMILES: CC1(C)c2cc3cc(-c4nc(-c5ccccc5)nc(-c5ccc6c(c5)c5ccccc5n6-c5ccccc5)n4)ccc3cc2-c2cc3oc(-c4ccccc4)nc3cc21 ; CC1(C)c2cc3cc(-c4nc(-c5ccccc5)nc(-c5ccccc5)n4)ccc3cc2-c2cc3oc(-c4ccc5ccccc5c4)nc3cc21]

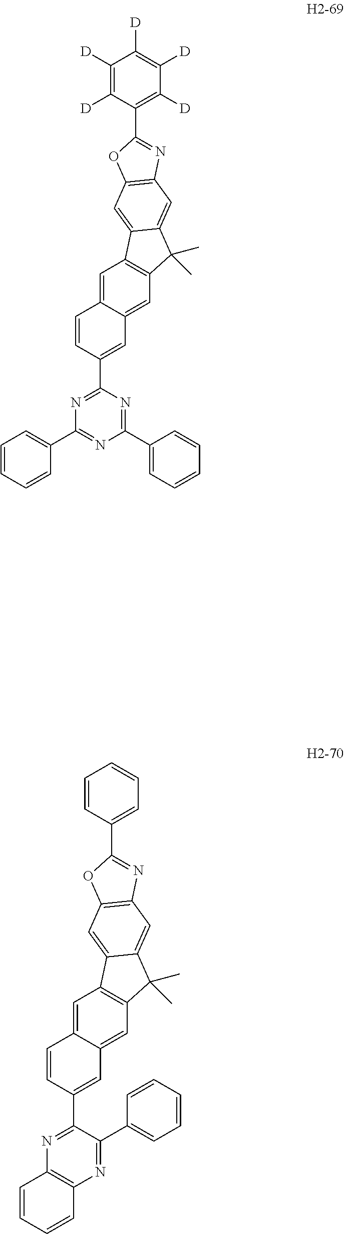 [SMILES: [2H]c1c([2H])c([2H])c(-c2nc3cc4c(cc3o2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3oc(-c4ccccc4)nc3cc21]

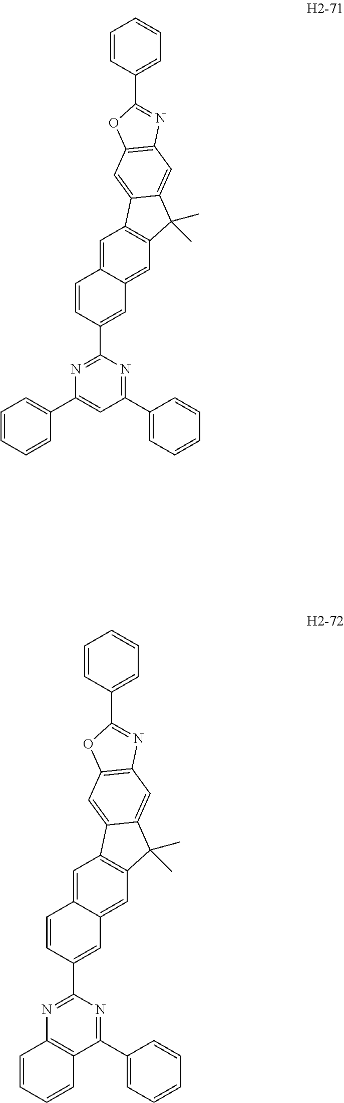 [SMILES: CC1(C)c2cc3cc(-c4nc(-c5ccccc5)cc(-c5ccccc5)n4)ccc3cc2-c2cc3oc(-c4ccccc4)nc3cc21 ; CC1(C)c2cc3cc(-c4nc(-c5ccccc5)c5ccccc5n4)ccc3cc2-c2cc3oc(-c4ccccc4)nc3cc21]

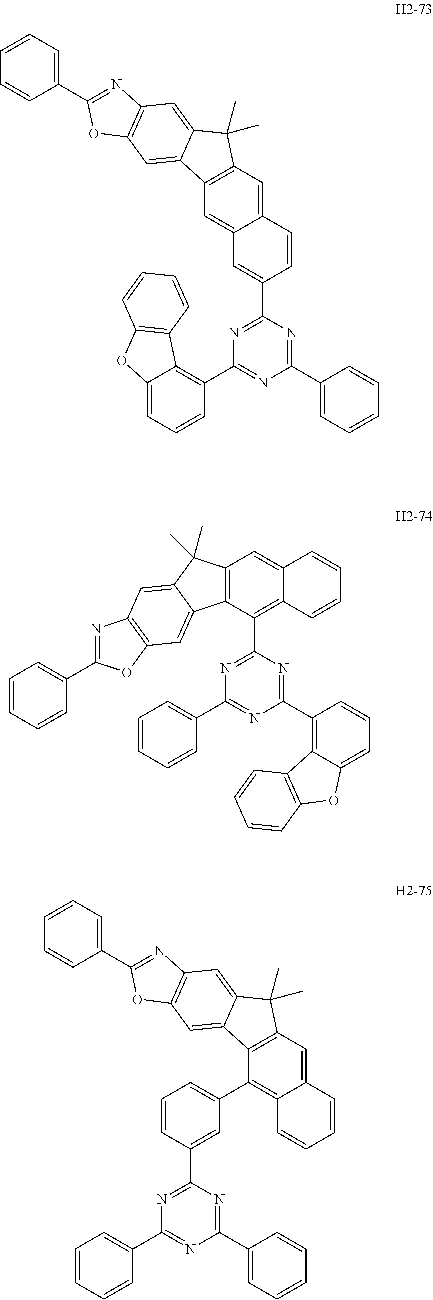 [SMILES: CC1(C)c2cc3ccc(-c4nc(-c5ccccc5)nc(-c5cccc6oc7ccccc7c56)n4)cc3cc2-c2cc3oc(-c4ccccc4)nc3cc21 ; CC1(C)c2cc3nc(-c4ccccc4)oc3cc2-c2c1cc1ccccc1c2-c1nc(-c2ccccc2)nc(-c2cccc3oc4ccccc4c23)n1 ; CC1(C)c2cc3nc(-c4ccccc4)oc3cc2-c2c1cc1ccccc1c2-c1cccc(-c2nc(-c3ccccc3)nc(-c3ccccc3)n2)c1]

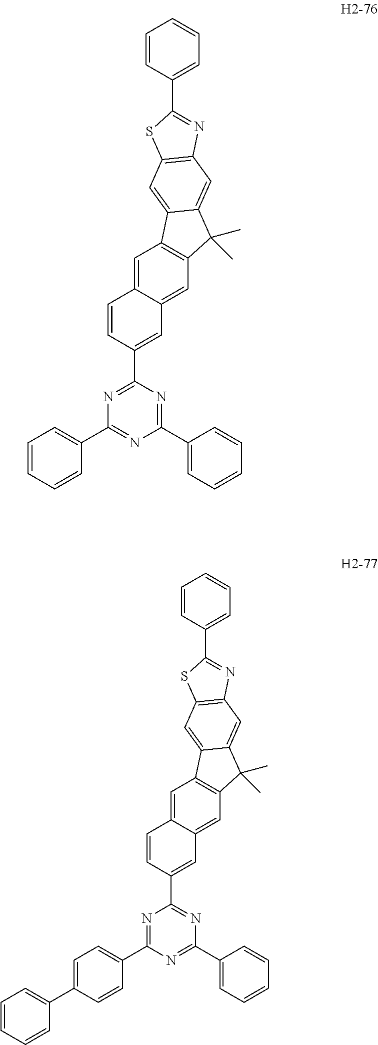 [SMILES: CC1(C)c2cc3cc(-c4nc(-c5ccccc5)nc(-c5ccccc5)n4)ccc3cc2-c2cc3sc(-c4ccccc4)nc3cc21 ; CC1(C)c2cc3cc(-c4nc(-c5ccccc5)nc(-c5ccc(-c6ccccc6)cc5)n4)ccc3cc2-c2cc3sc(-c4ccccc4)nc3cc21]

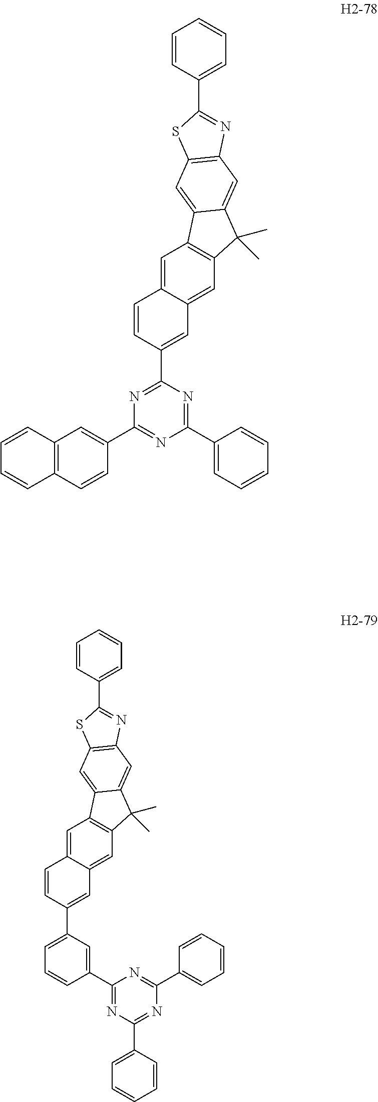 [SMILES: CC1(C)c2cc3cc(-c4nc(-c5ccccc5)nc(-c5ccc6ccccc6c5)n4)ccc3cc2-c2cc3sc(-c4ccccc4)nc3cc21 ; CC1(C)c2cc3cc(-c4cccc(-c5nc(-c6ccccc6)nc(-c6ccccc6)n5)c4)ccc3cc2-c2cc3sc(-c4ccccc4)nc3cc21]

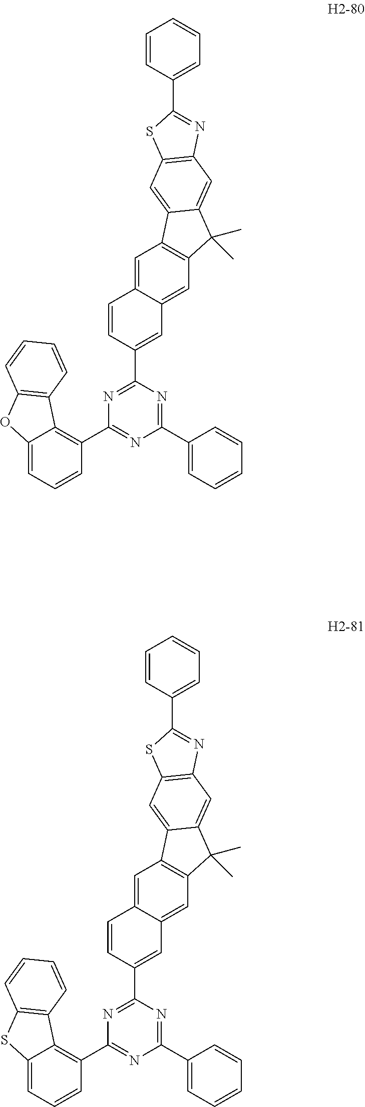 [SMILES: CC1(C)c2cc3cc(-c4nc(-c5ccccc5)nc(-c5cccc6oc7ccccc7c56)n4)ccc3cc2-c2cc3sc(-c4ccccc4)nc3cc21 ; CC1(C)c2cc3cc(-c4nc(-c5ccccc5)nc(-c5cccc6sc7ccccc7c56)n4)ccc3cc2-c2cc3sc(-c4ccccc4)nc3cc21]

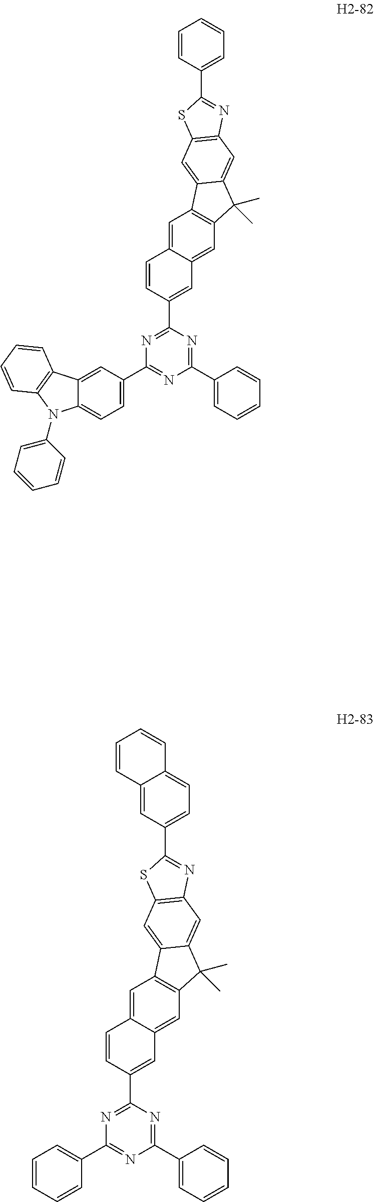 [SMILES: CC1(C)c2cc3cc(-c4nc(-c5ccccc5)nc(-c5ccc6c(c5)c5ccccc5n6-c5ccccc5)n4)ccc3cc2-c2cc3sc(-c4ccccc4)nc3cc21 ; CC1(C)c2cc3cc(-c4nc(-c5ccccc5)nc(-c5ccccc5)n4)ccc3cc2-c2cc3sc(-c4ccc5ccccc5c4)nc3cc21]

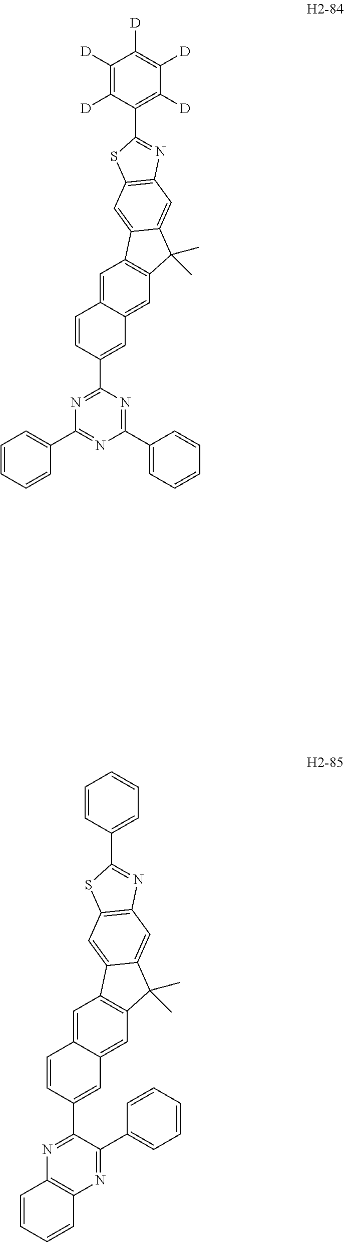 [SMILES: [2H]c1c([2H])c([2H])c(-c2nc3cc4c(cc3s2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3sc(-c4ccccc4)nc3cc21]

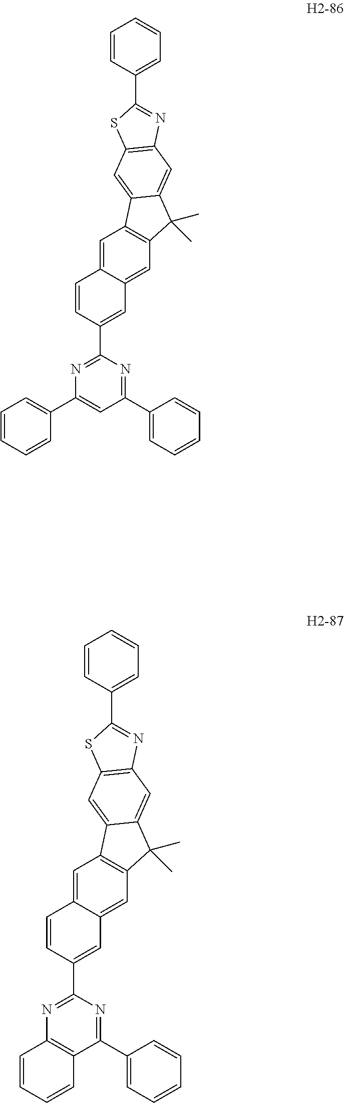 [SMILES: CC1(C)c2cc3cc(-c4nc(-c5ccccc5)cc(-c5ccccc5)n4)ccc3cc2-c2cc3sc(-c4ccccc4)nc3cc21 ; CC1(C)c2cc3cc(-c4nc(-c5ccccc5)c5ccccc5n4)ccc3cc2-c2cc3sc(-c4ccccc4)nc3cc21]

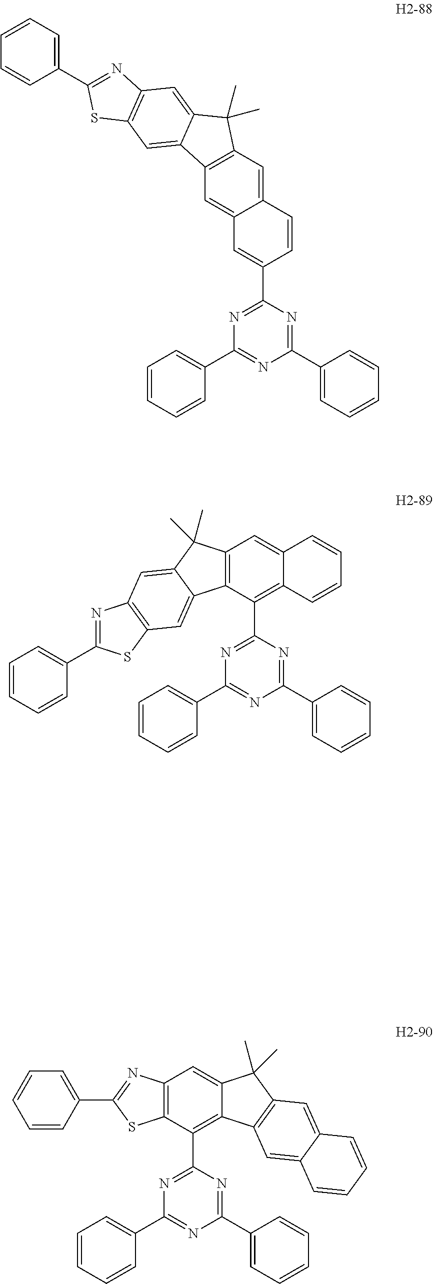 [SMILES: CC1(C)c2cc3ccc(-c4nc(-c5ccccc5)nc(-c5ccccc5)n4)cc3cc2-c2cc3sc(-c4ccccc4)nc3cc21 ; CC1(C)c2cc3nc(-c4ccccc4)sc3cc2-c2c1cc1ccccc1c2-c1nc(-c2ccccc2)nc(-c2ccccc2)n1 ; CC1(C)c2cc3ccccc3cc2-c2c1cc1nc(-c3ccccc3)sc1c2-c1nc(-c2ccccc2)nc(-c2ccccc2)n1]

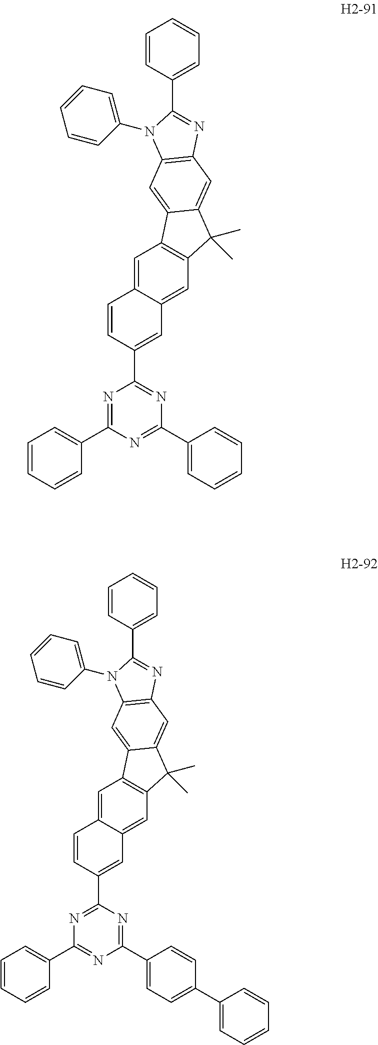 [SMILES: CC1(C)c2cc3cc(-c4nc(-c5ccccc5)nc(-c5ccccc5)n4)ccc3cc2-c2cc3c(cc21)nc(-c1ccccc1)n3-c1ccccc1 ; CC1(C)c2cc3cc(-c4nc(-c5ccccc5)nc(-c5ccc(-c6ccccc6)cc5)n4)ccc3cc2-c2cc3c(cc21)nc(-c1ccccc1)n3-c1ccccc1]

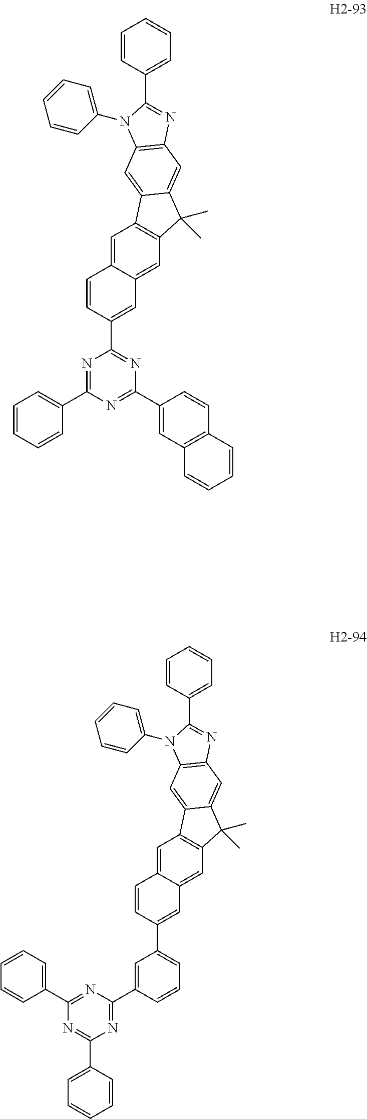 [SMILES: CC1(C)c2cc3cc(-c4nc(-c5ccccc5)nc(-c5ccc6ccccc6c5)n4)ccc3cc2-c2cc3c(cc21)nc(-c1ccccc1)n3-c1ccccc1 ; CC1(C)c2cc3cc(-c4cccc(-c5nc(-c6ccccc6)nc(-c6ccccc6)n5)c4)ccc3cc2-c2cc3c(cc21)nc(-c1ccccc1)n3-c1ccccc1]

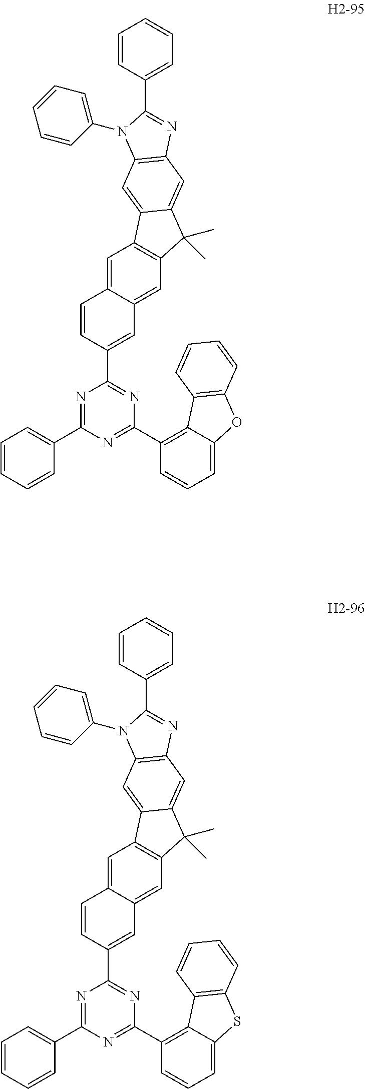 [SMILES: CC1(C)c2cc3cc(-c4nc(-c5ccccc5)nc(-c5cccc6oc7ccccc7c56)n4)ccc3cc2-c2cc3c(cc21)nc(-c1ccccc1)n3-c1ccccc1 ; CC1(C)c2cc3cc(-c4nc(-c5ccccc5)nc(-c5cccc6sc7ccccc7c56)n4)ccc3cc2-c2cc3c(cc21)nc(-c1ccccc1)n3-c1ccccc1]

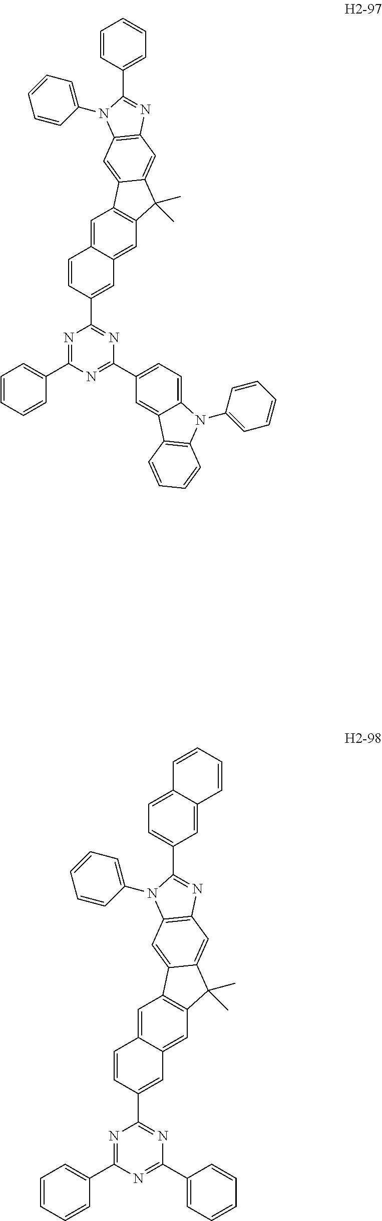 [SMILES: CC1(C)c2cc3cc(-c4nc(-c5ccccc5)nc(-c5ccc6c(c5)c5ccccc5n6-c5ccccc5)n4)ccc3cc2-c2cc3c(cc21)nc(-c1ccccc1)n3-c1ccccc1 ; CC1(C)c2cc3cc(-c4nc(-c5ccccc5)nc(-c5ccccc5)n4)ccc3cc2-c2cc3c(cc21)nc(-c1ccc2ccccc2c1)n3-c1ccccc1]

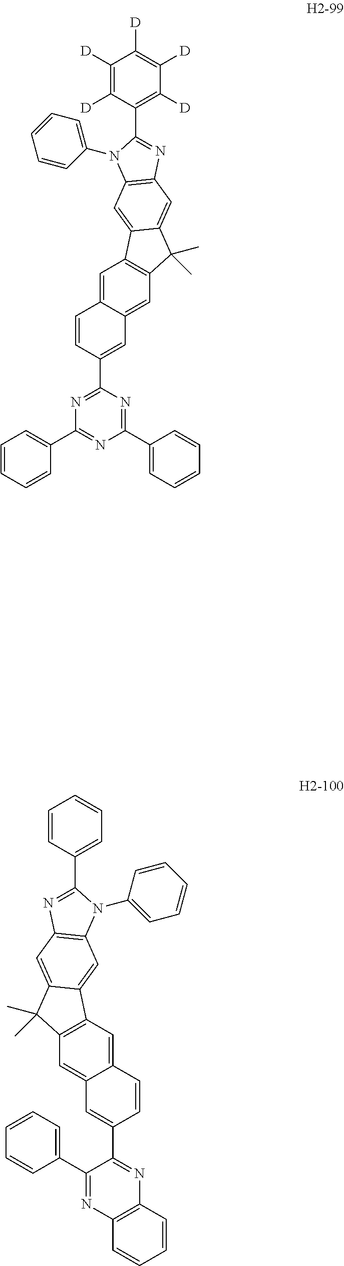 [SMILES: [2H]c1c([2H])c([2H])c(-c2nc3cc4c(cc3n2-c2ccccc2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3c(cc21)nc(-c1ccccc1)n3-c1ccccc1]

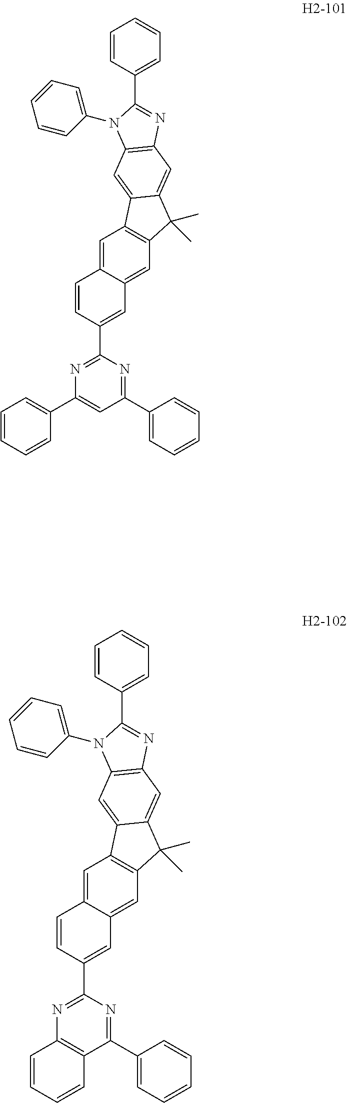 [SMILES: CC1(C)c2cc3cc(-c4nc(-c5ccccc5)cc(-c5ccccc5)n4)ccc3cc2-c2cc3c(cc21)nc(-c1ccccc1)n3-c1ccccc1 ; CC1(C)c2cc3cc(-c4nc(-c5ccccc5)c5ccccc5n4)ccc3cc2-c2cc3c(cc21)nc(-c1ccccc1)n3-c1ccccc1]

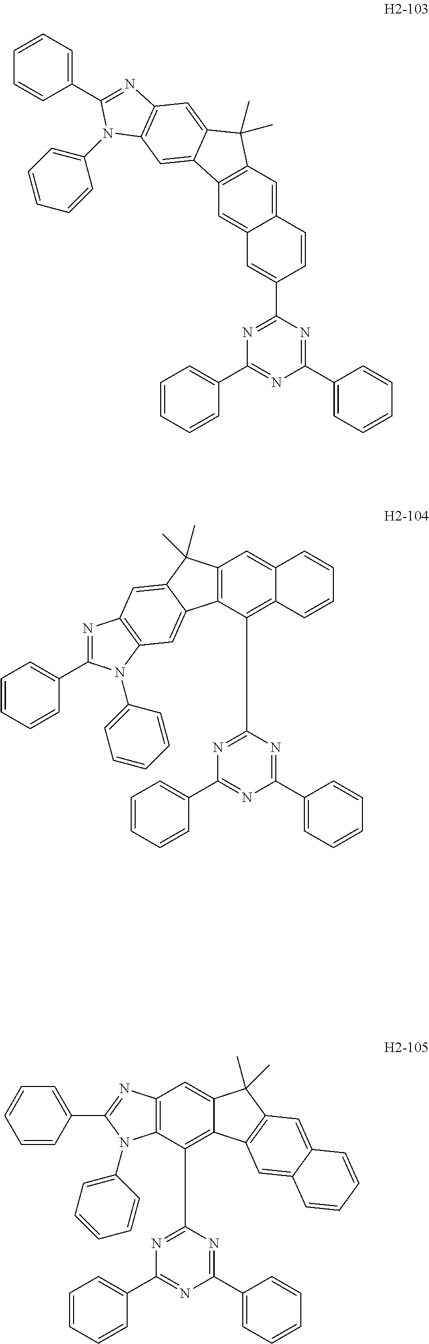 [SMILES: CC1(C)c2cc3ccc(-c4nc(-c5ccccc5)nc(-c5ccccc5)n4)cc3cc2-c2cc3c(cc21)nc(-c1ccccc1)n3-c1ccccc1 ; CC1(C)c2cc3nc(-c4ccccc4)n(-c4ccccc4)c3cc2-c2c1cc1ccccc1c2-c1nc(-c2ccccc2)nc(-c2ccccc2)n1 ; CC1(C)c2cc3ccccc3cc2-c2c1cc1nc(-c3ccccc3)n(-c3ccccc3)c1c2-c1nc(-c2ccccc2)nc(-c2ccccc2)n1]

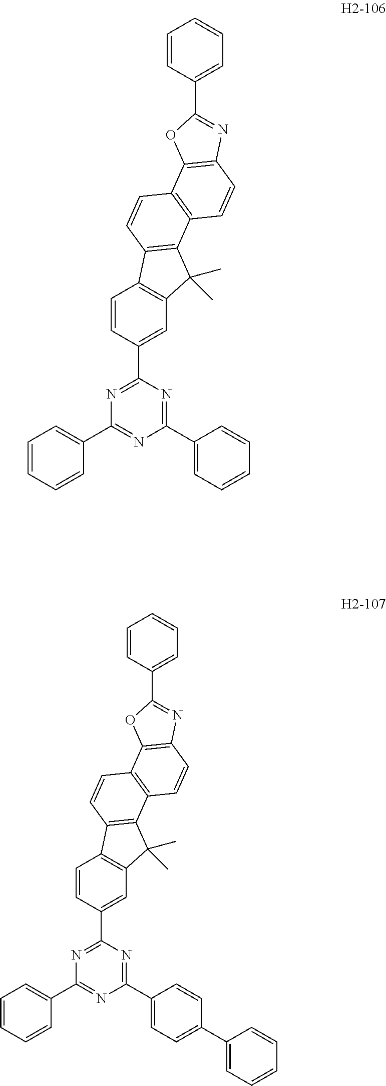 [SMILES: CC1(C)c2cc(-c3nc(-c4ccccc4)nc(-c4ccccc4)n3)ccc2-c2ccc3c(ccc4nc(-c5ccccc5)oc43)c21 ; CC1(C)c2cc(-c3nc(-c4ccccc4)nc(-c4ccc(-c5ccccc5)cc4)n3)ccc2-c2ccc3c(ccc4nc(-c5ccccc5)oc43)c21]

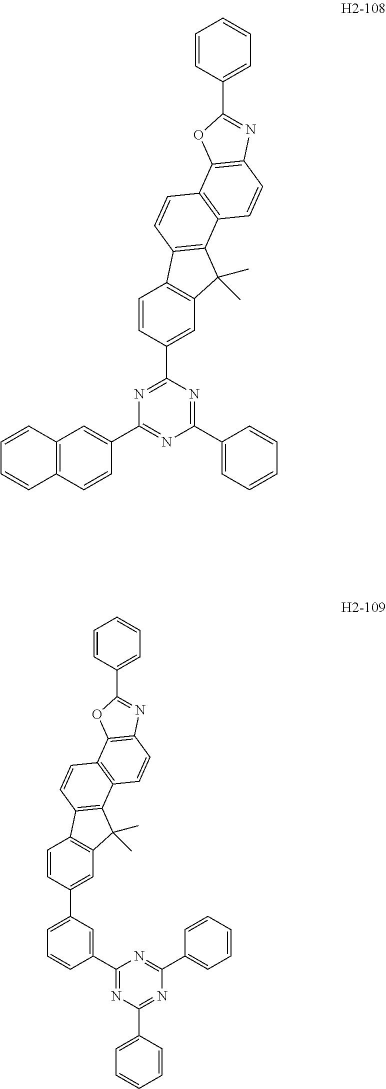 [SMILES: CC1(C)c2cc(-c3nc(-c4ccccc4)nc(-c4ccc5ccccc5c4)n3)ccc2-c2ccc3c(ccc4nc(-c5ccccc5)oc43)c21 ; CC1(C)c2cc(-c3cccc(-c4nc(-c5ccccc5)nc(-c5ccccc5)n4)c3)ccc2-c2ccc3c(ccc4nc(-c5ccccc5)oc43)c21]

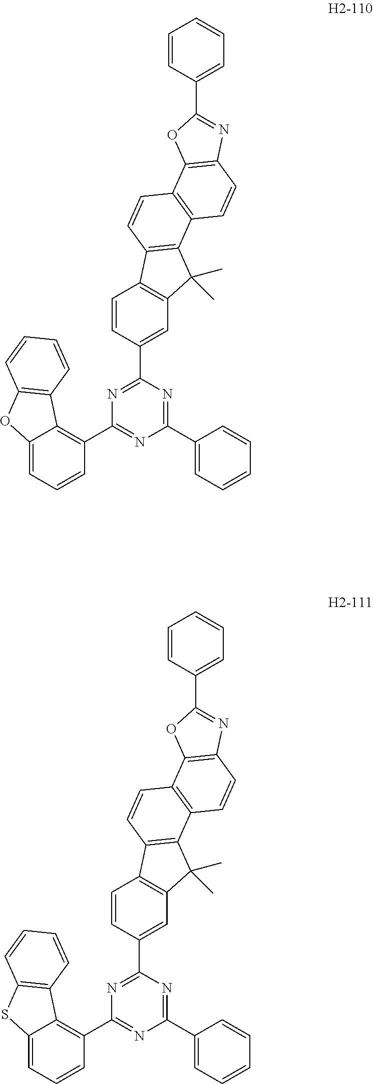 [SMILES: CC1(C)c2cc(-c3nc(-c4ccccc4)nc(-c4cccc5oc6ccccc6c45)n3)ccc2-c2ccc3c(ccc4nc(-c5ccccc5)oc43)c21 ; CC1(C)c2cc(-c3nc(-c4ccccc4)nc(-c4cccc5sc6ccccc6c45)n3)ccc2-c2ccc3c(ccc4nc(-c5ccccc5)oc43)c21]

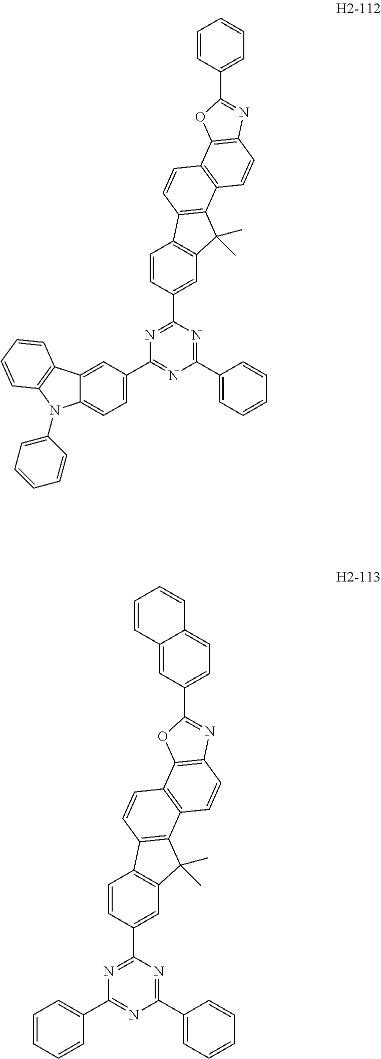 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)oc43)c21 ; CC1(C)c2cc(-c3nc(-c4ccccc4)nc(-c4ccccc4)n3)ccc2-c2ccc3c(ccc4nc(-c5ccc6ccccc6c5)oc43)c21]

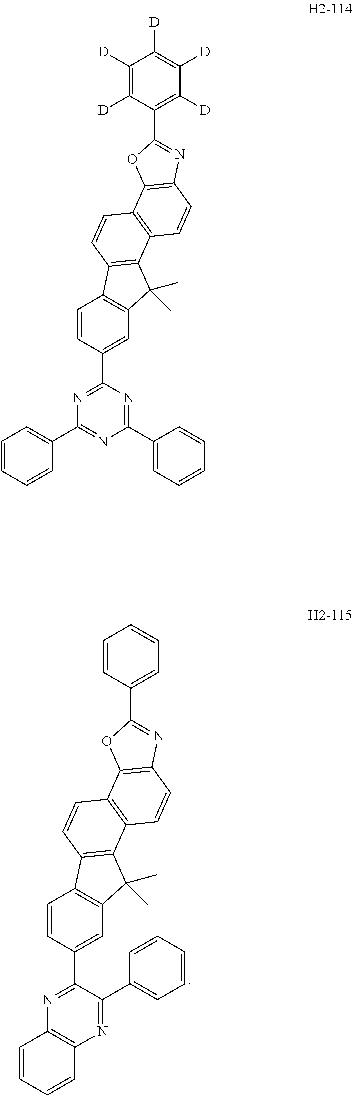 [SMILES: [2H]c1c([2H])c([2H])c(-c2nc3ccc4c5c(ccc4c3o2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)oc43)c21]

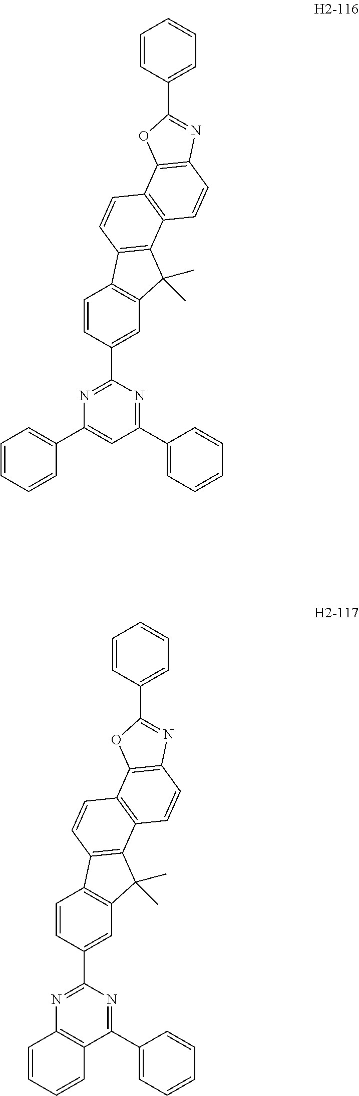 [SMILES: CC1(C)c2cc(-c3nc(-c4ccccc4)cc(-c4ccccc4)n3)ccc2-c2ccc3c(ccc4nc(-c5ccccc5)oc43)c21 ; CC1(C)c2cc(-c3nc(-c4ccccc4)c4ccccc4n3)ccc2-c2ccc3c(ccc4nc(-c5ccccc5)oc43)c21]

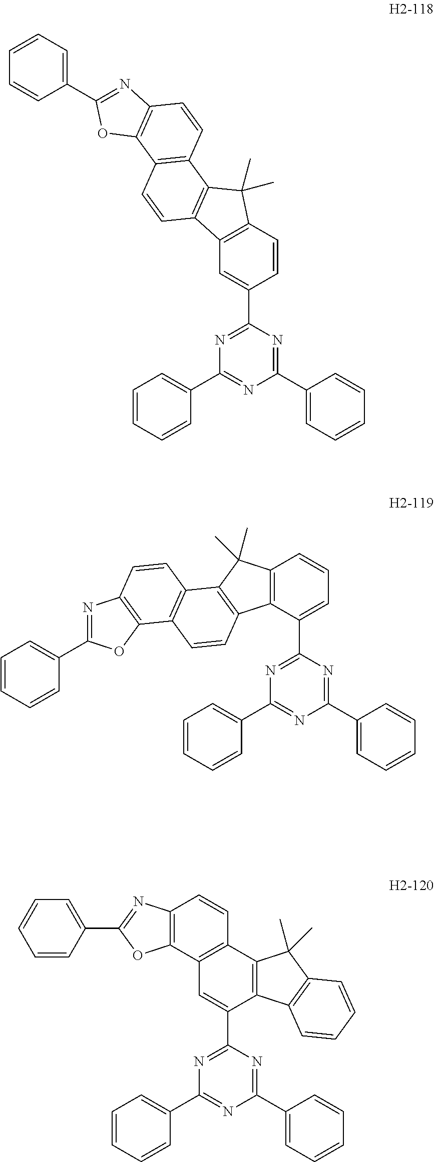 [SMILES: CC1(C)c2ccc(-c3nc(-c4ccccc4)nc(-c4ccccc4)n3)cc2-c2ccc3c(ccc4nc(-c5ccccc5)oc43)c21 ; CC1(C)c2cccc(-c3nc(-c4ccccc4)nc(-c4ccccc4)n3)c2-c2ccc3c(ccc4nc(-c5ccccc5)oc43)c21 ; CC1(C)c2ccccc2-c2c(-c3nc(-c4ccccc4)nc(-c4ccccc4)n3)cc3c(ccc4nc(-c5ccccc5)oc43)c21]

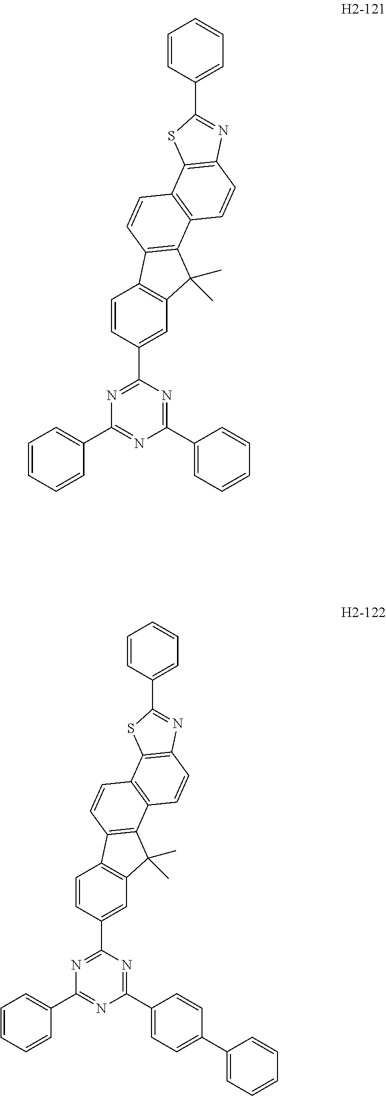 [SMILES: CC1(C)c2cc(-c3nc(-c4ccccc4)nc(-c4ccccc4)n3)ccc2-c2ccc3c(ccc4nc(-c5ccccc5)sc43)c21 ; CC1(C)c2cc(-c3nc(-c4ccccc4)nc(-c4ccc(-c5ccccc5)cc4)n3)ccc2-c2ccc3c(ccc4nc(-c5ccccc5)sc43)c21]

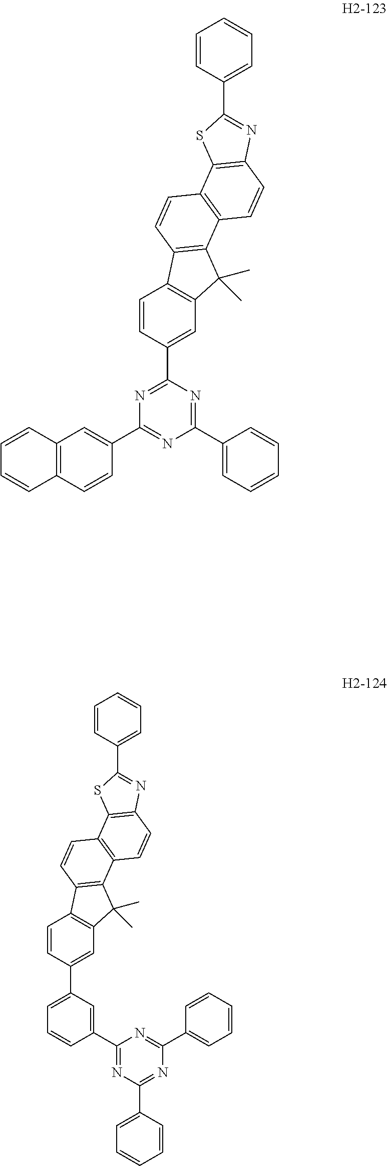 [SMILES: CC1(C)c2cc(-c3nc(-c4ccccc4)nc(-c4ccc5ccccc5c4)n3)ccc2-c2ccc3c(ccc4nc(-c5ccccc5)sc43)c21 ; CC1(C)c2cc(-c3cccc(-c4nc(-c5ccccc5)nc(-c5ccccc5)n4)c3)ccc2-c2ccc3c(ccc4nc(-c5ccccc5)sc43)c21]

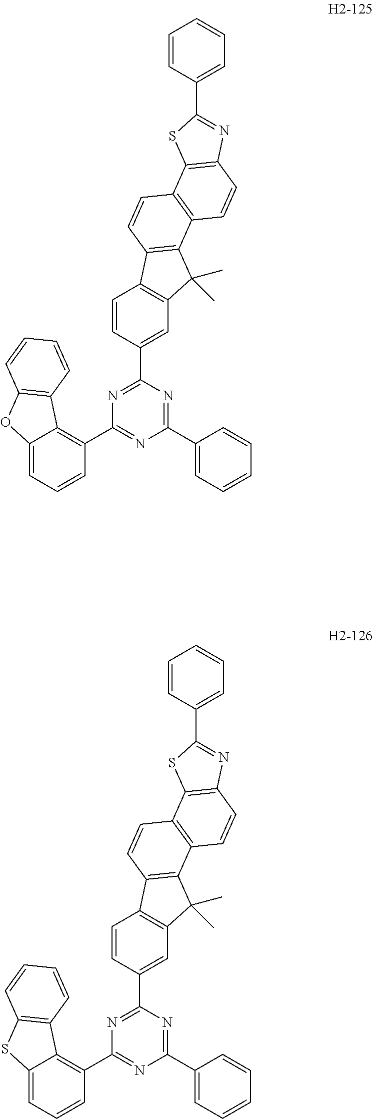 [SMILES: CC1(C)c2cc(-c3nc(-c4ccccc4)nc(-c4cccc5oc6ccccc6c45)n3)ccc2-c2ccc3c(ccc4nc(-c5ccccc5)sc43)c21 ; CC1(C)c2cc(-c3nc(-c4ccccc4)nc(-c4cccc5sc6ccccc6c45)n3)ccc2-c2ccc3c(ccc4nc(-c5ccccc5)sc43)c21]

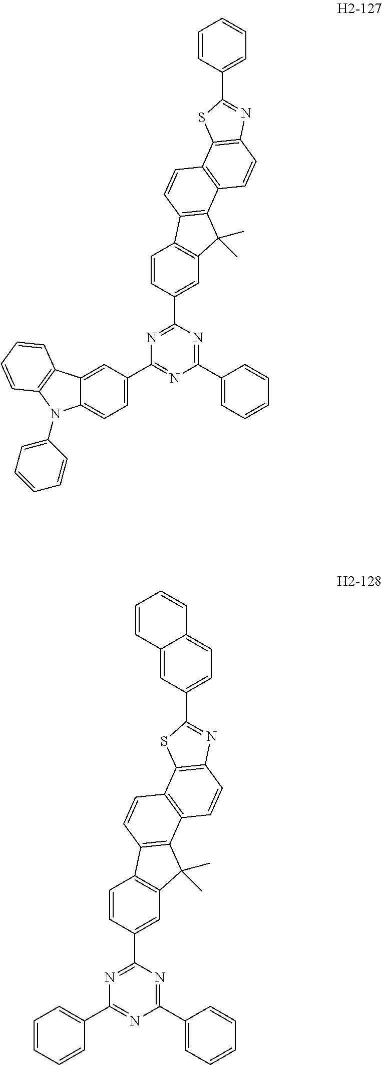 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)sc43)c21 ; CC1(C)c2cc(-c3nc(-c4ccccc4)nc(-c4ccccc4)n3)ccc2-c2ccc3c(ccc4nc(-c5ccc6ccccc6c5)sc43)c21]

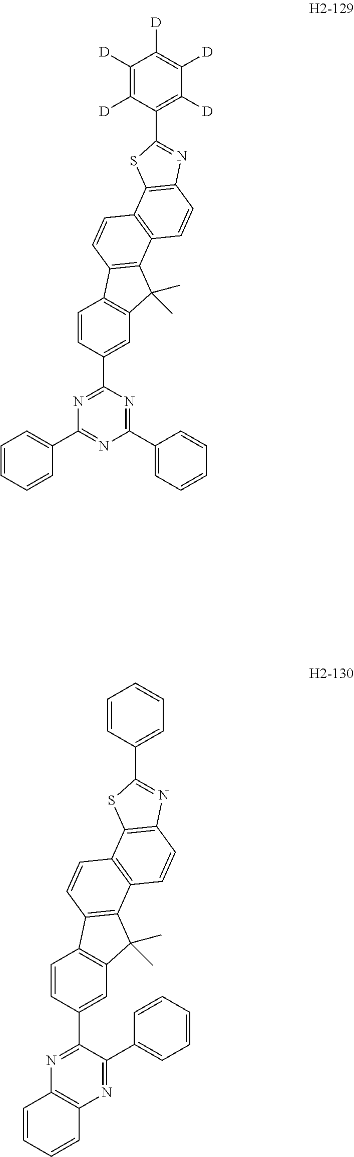 [SMILES: [2H]c1c([2H])c([2H])c(-c2nc3ccc4c5c(ccc4c3s2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)sc43)c21]

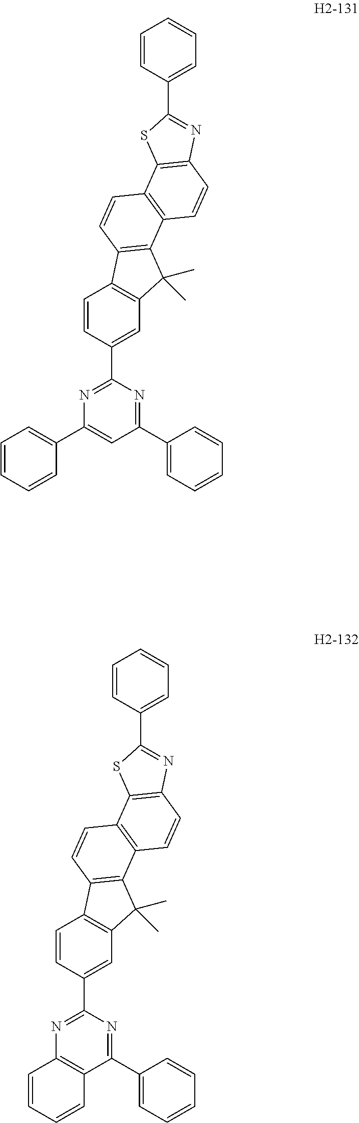 [SMILES: CC1(C)c2cc(-c3nc(-c4ccccc4)cc(-c4ccccc4)n3)ccc2-c2ccc3c(ccc4nc(-c5ccccc5)sc43)c21 ; CC1(C)c2cc(-c3nc(-c4ccccc4)c4ccccc4n3)ccc2-c2ccc3c(ccc4nc(-c5ccccc5)sc43)c21]

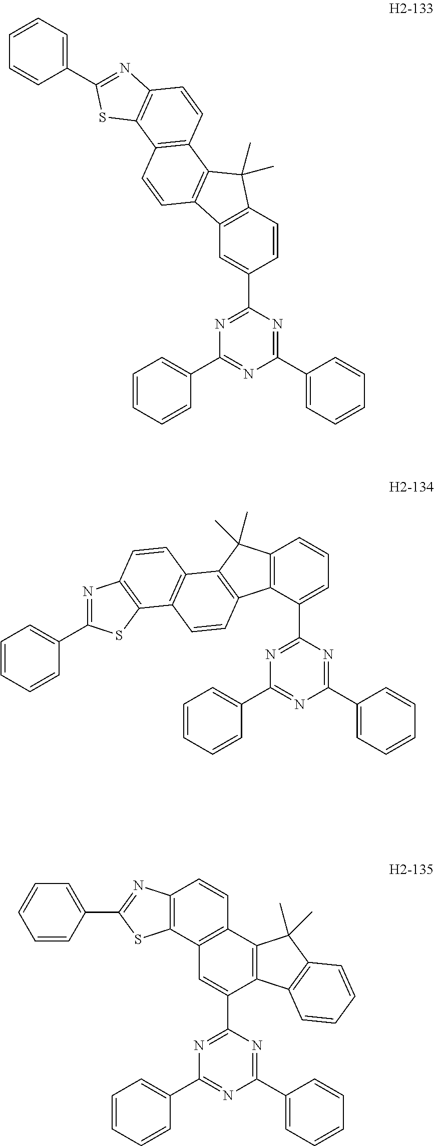 [SMILES: CC1(C)c2ccc(-c3nc(-c4ccccc4)nc(-c4ccccc4)n3)cc2-c2ccc3c(ccc4nc(-c5ccccc5)sc43)c21 ; CC1(C)c2cccc(-c3nc(-c4ccccc4)nc(-c4ccccc4)n3)c2-c2ccc3c(ccc4nc(-c5ccccc5)sc43)c21 ; CC1(C)c2ccccc2-c2c(-c3nc(-c4ccccc4)nc(-c4ccccc4)n3)cc3c(ccc4nc(-c5ccccc5)sc43)c21]

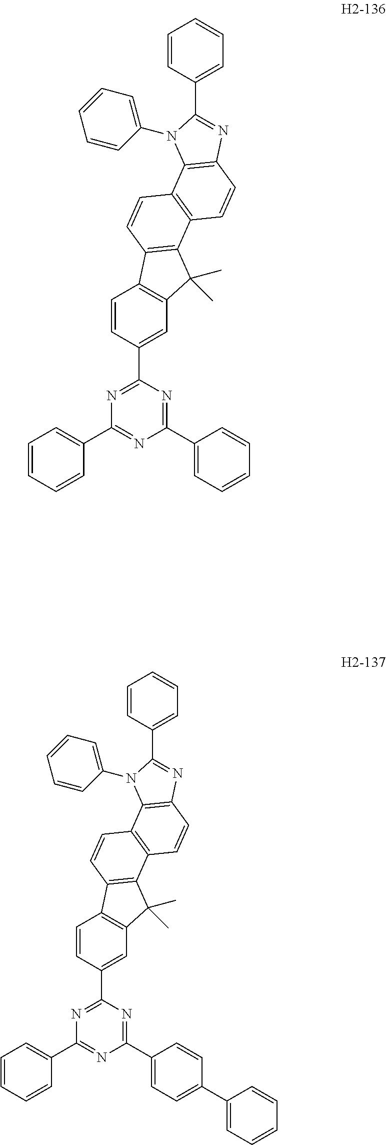 [SMILES: CC1(C)c2cc(-c3nc(-c4ccccc4)nc(-c4ccccc4)n3)ccc2-c2ccc3c(ccc4nc(-c5ccccc5)n(-c5ccccc5)c43)c21 ; CC1(C)c2cc(-c3nc(-c4ccccc4)nc(-c4ccc(-c5ccccc5)cc4)n3)ccc2-c2ccc3c(ccc4nc(-c5ccccc5)n(-c5ccccc5)c43)c21]

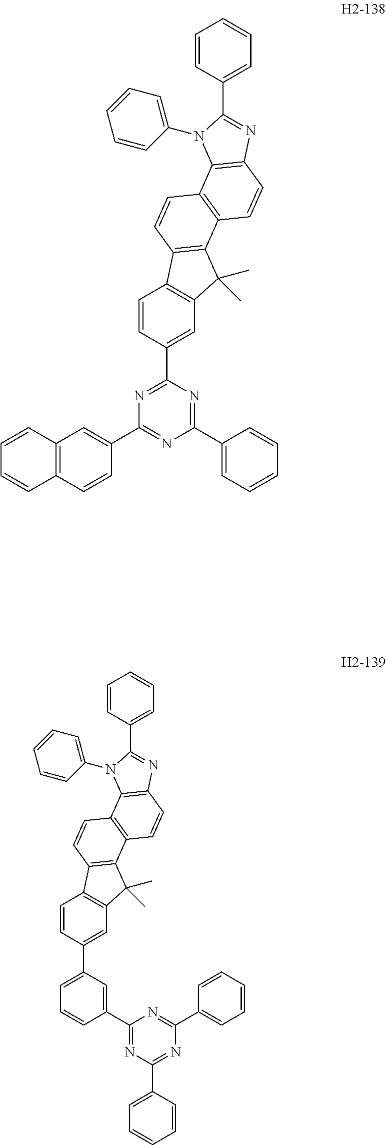 [SMILES: CC1(C)c2cc(-c3nc(-c4ccccc4)nc(-c4ccc5ccccc5c4)n3)ccc2-c2ccc3c(ccc4nc(-c5ccccc5)n(-c5ccccc5)c43)c21 ; CC1(C)c2cc(-c3cccc(-c4nc(-c5ccccc5)nc(-c5ccccc5)n4)c3)ccc2-c2ccc3c(ccc4nc(-c5ccccc5)n(-c5ccccc5)c43)c21]

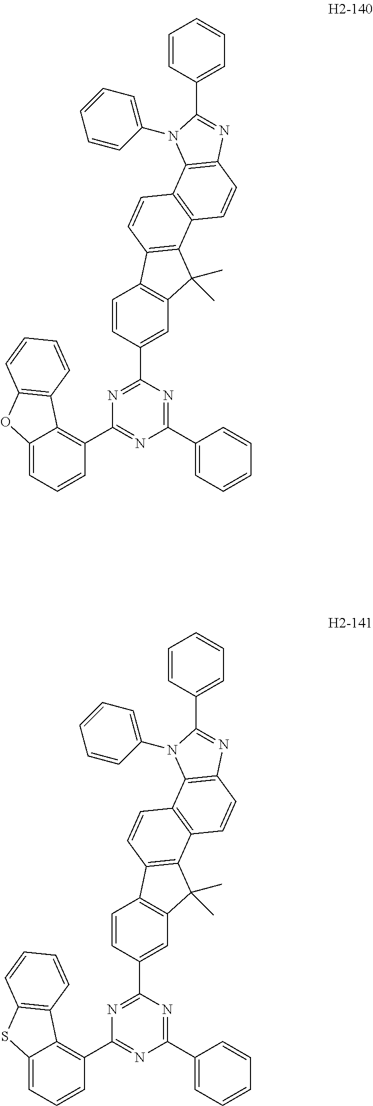 [SMILES: CC1(C)c2cc(-c3nc(-c4ccccc4)nc(-c4cccc5oc6ccccc6c45)n3)ccc2-c2ccc3c(ccc4nc(-c5ccccc5)n(-c5ccccc5)c43)c21 ; CC1(C)c2cc(-c3nc(-c4ccccc4)nc(-c4cccc5sc6ccccc6c45)n3)ccc2-c2ccc3c(ccc4nc(-c5ccccc5)n(-c5ccccc5)c43)c21]

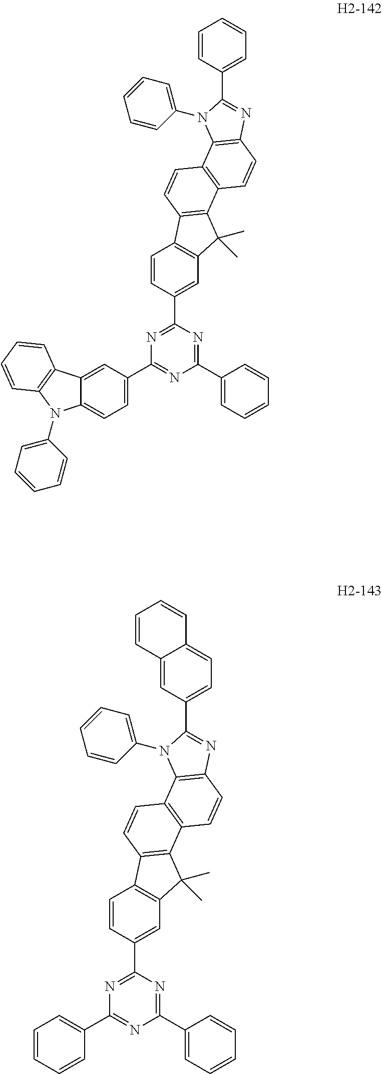 [SMILES: CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)n(-c5ccccc5)c43)c21 ; CC1(C)c2cc(-c3nc(-c4ccccc4)nc(-c4ccccc4)n3)ccc2-c2ccc3c(ccc4nc(-c5ccc6ccccc6c5)n(-c5ccccc5)c43)c21]

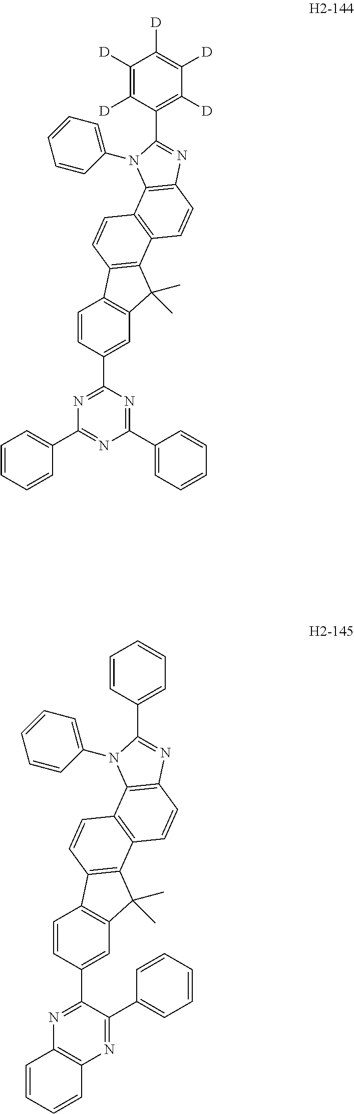 [SMILES: [2H]c1c([2H])c([2H])c(-c2nc3ccc4c5c(ccc4c3n2-c2ccccc2)-c2ccc(-c3nc(-c4ccccc4)nc(-c4ccccc4)n3)cc2C5(C)C)c([2H])c1[2H] ; CC1(C)c2cc(-c3nc4ccccc4nc3-c3ccccc3)ccc2-c2ccc3c(ccc4nc(-c5ccccc5)n(-c5ccccc5)c43)c21]

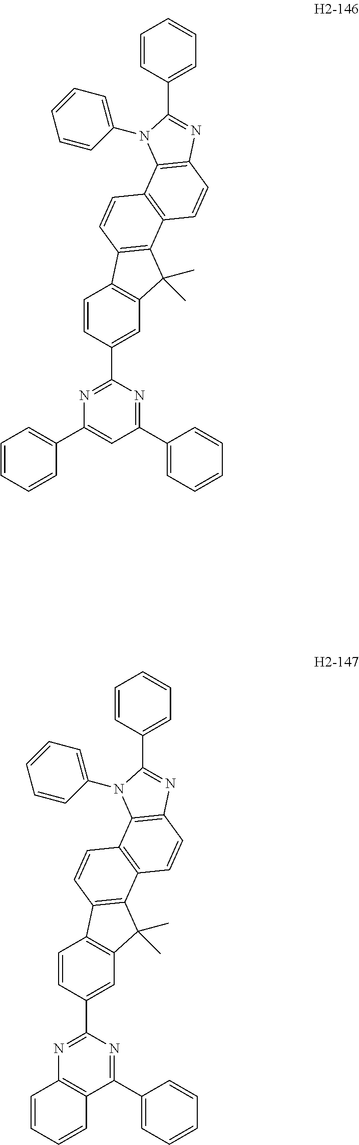 [SMILES: CC1(C)c2cc(-c3nc(-c4ccccc4)cc(-c4ccccc4)n3)ccc2-c2ccc3c(ccc4nc(-c5ccccc5)n(-c5ccccc5)c43)c21 ; CC1(C)c2cc(-c3nc(-c4ccccc4)c4ccccc4n3)ccc2-c2ccc3c(ccc4nc(-c5ccccc5)n(-c5ccccc5)c43)c21]

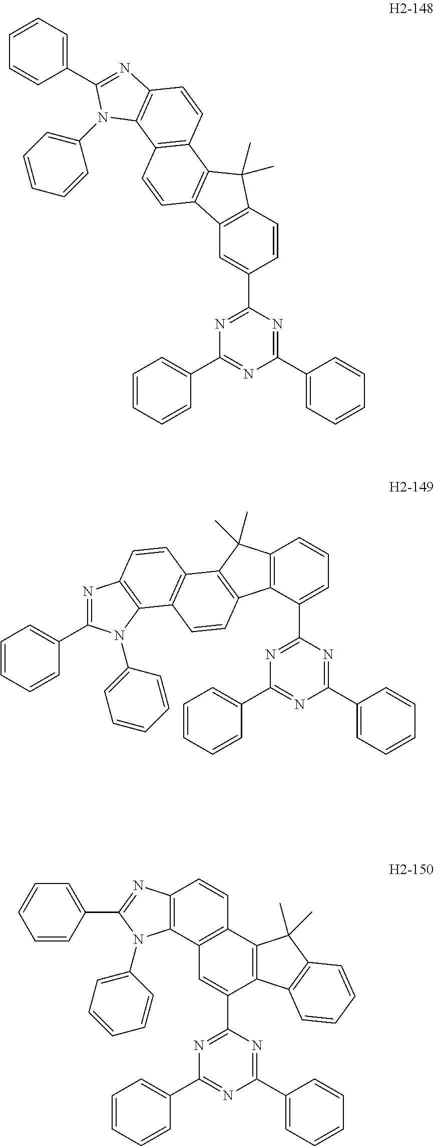 [SMILES: CC1(C)c2ccc(-c3nc(-c4ccccc4)nc(-c4ccccc4)n3)cc2-c2ccc3c(ccc4nc(-c5ccccc5)n(-c5ccccc5)c43)c21 ; CC1(C)c2cccc(-c3nc(-c4ccccc4)nc(-c4ccccc4)n3)c2-c2ccc3c(ccc4nc(-c5ccccc5)n(-c5ccccc5)c43)c21 ; CC1(C)c2ccccc2-c2c(-c3nc(-c4ccccc4)nc(-c4ccccc4)n3)cc3c(ccc4nc(-c5ccccc5)n(-c5ccccc5)c43)c21]

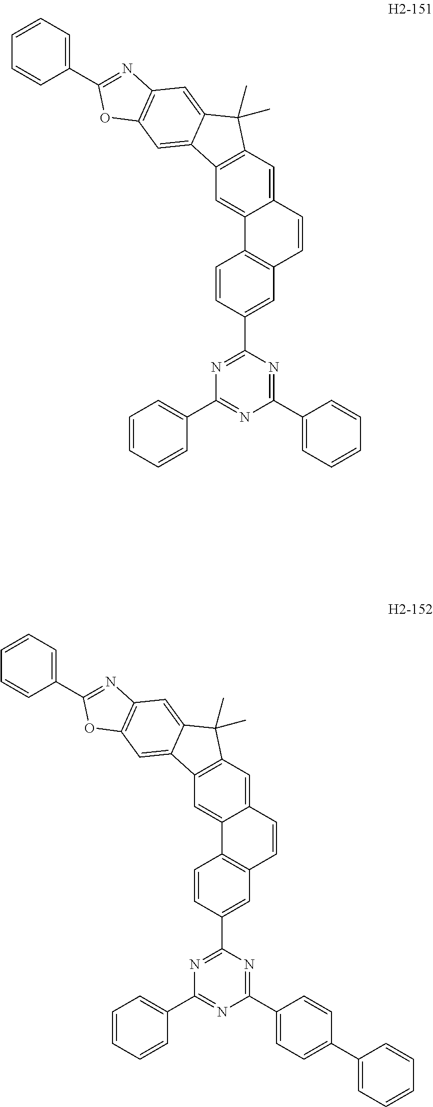 [SMILES: CC1(C)c2cc3ccc4cc(-c5nc(-c6ccccc6)nc(-c6ccccc6)n5)ccc4c3cc2-c2cc3oc(-c4ccccc4)nc3cc21 ; CC1(C)c2cc3ccc4cc(-c5nc(-c6ccccc6)nc(-c6ccc(-c7ccccc7)cc6)n5)ccc4c3cc2-c2cc3oc(-c4ccccc4)nc3cc21]

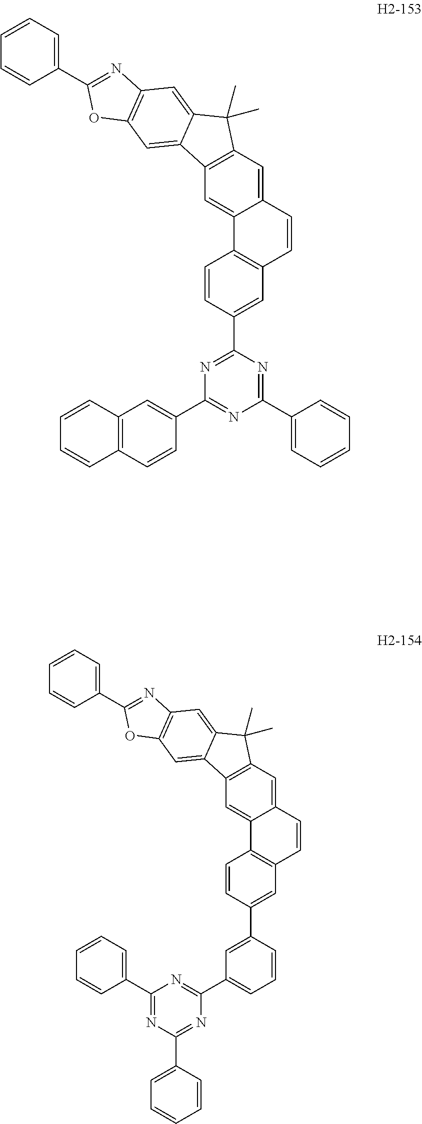 [SMILES: CC1(C)c2cc3ccc4cc(-c5nc(-c6ccccc6)nc(-c6ccc7ccccc7c6)n5)ccc4c3cc2-c2cc3oc(-c4ccccc4)nc3cc21 ; CC1(C)c2cc3ccc4cc(-c5cccc(-c6nc(-c7ccccc7)nc(-c7ccccc7)n6)c5)ccc4c3cc2-c2cc3oc(-c4ccccc4)nc3cc21]

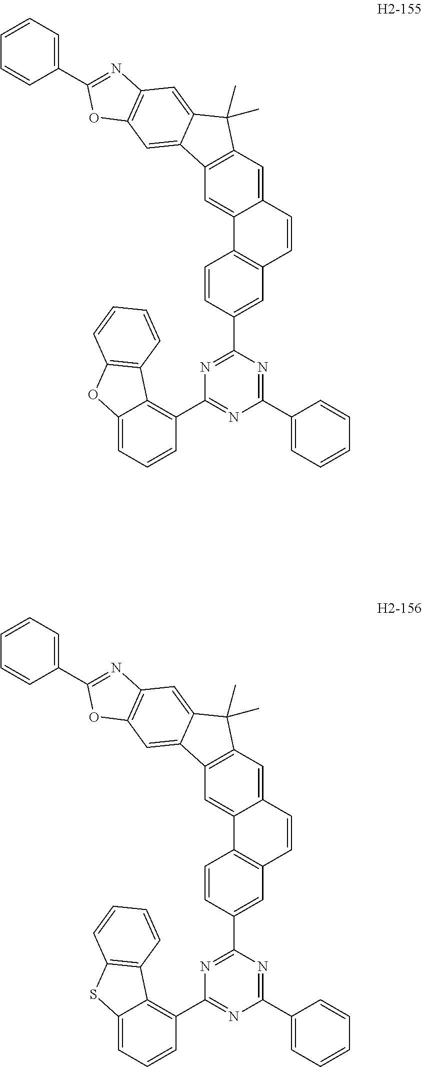 [SMILES: CC1(C)c2cc3ccc4cc(-c5nc(-c6ccccc6)nc(-c6cccc7oc8ccccc8c67)n5)ccc4c3cc2-c2cc3oc(-c4ccccc4)nc3cc21 ; CC1(C)c2cc3ccc4cc(-c5nc(-c6ccccc6)nc(-c6cccc7sc8ccccc8c67)n5)ccc4c3cc2-c2cc3oc(-c4ccccc4)nc3cc21]

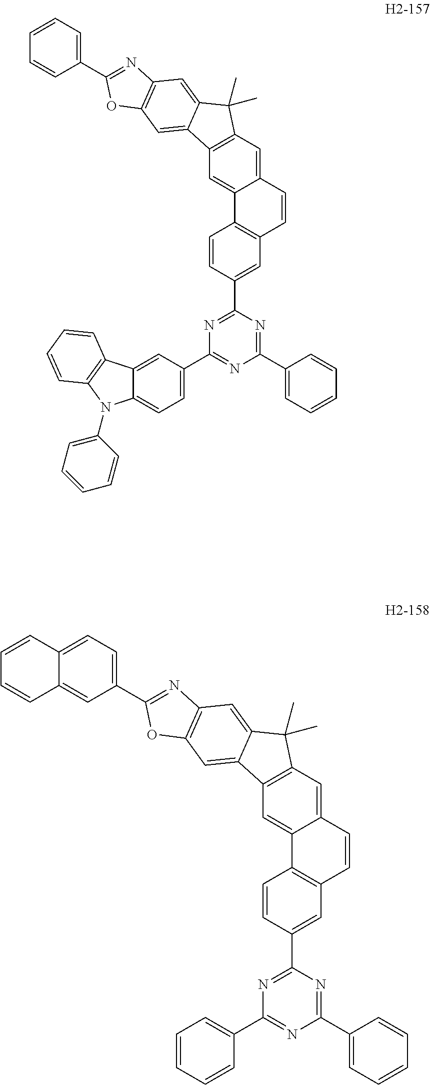 [SMILES: CC1(C)c2cc3ccc4cc(-c5nc(-c6ccccc6)nc(-c6ccc7c(c6)c6ccccc6n7-c6ccccc6)n5)ccc4c3cc2-c2cc3oc(-c4ccccc4)nc3cc21 ; CC1(C)c2cc3ccc4cc(-c5nc(-c6ccccc6)nc(-c6ccccc6)n5)ccc4c3cc2-c2cc3oc(-c4ccc5ccccc5c4)nc3cc21]

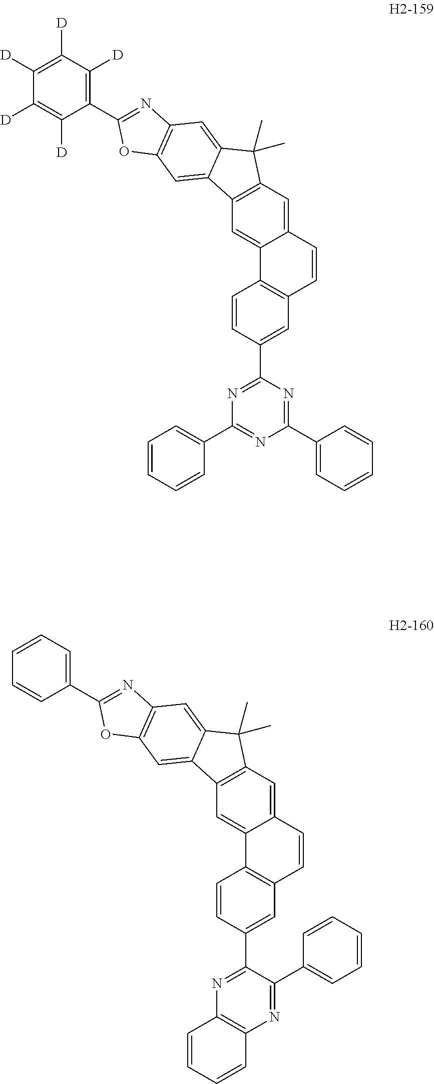 [SMILES: [2H]c1c([2H])c([2H])c(-c2nc3cc4c(cc3o2)-c2cc3c(ccc5cc(-c6nc(-c7ccccc7)nc(-c7ccccc7)n6)ccc53)cc2C4(C)C)c([2H])c1[2H] ; CC1(C)c2cc3ccc4cc(-c5nc6ccccc6nc5-c5ccccc5)ccc4c3cc2-c2cc3oc(-c4ccccc4)nc3cc21]

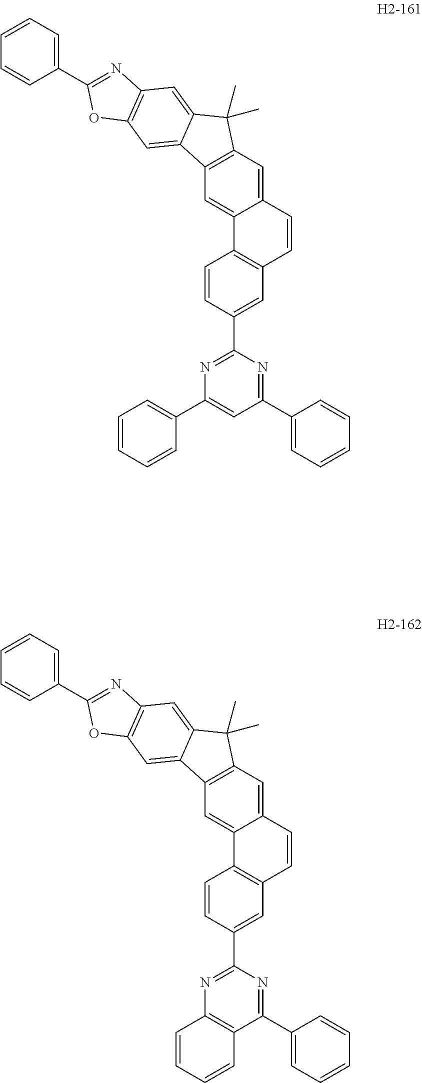 [SMILES: CC1(C)c2cc3ccc4cc(-c5nc(-c6ccccc6)cc(-c6ccccc6)n5)ccc4c3cc2-c2cc3oc(-c4ccccc4)nc3cc21 ; CC1(C)c2cc3ccc4cc(-c5nc(-c6ccccc6)c6ccccc6n5)ccc4c3cc2-c2cc3oc(-c4ccccc4)nc3cc21]

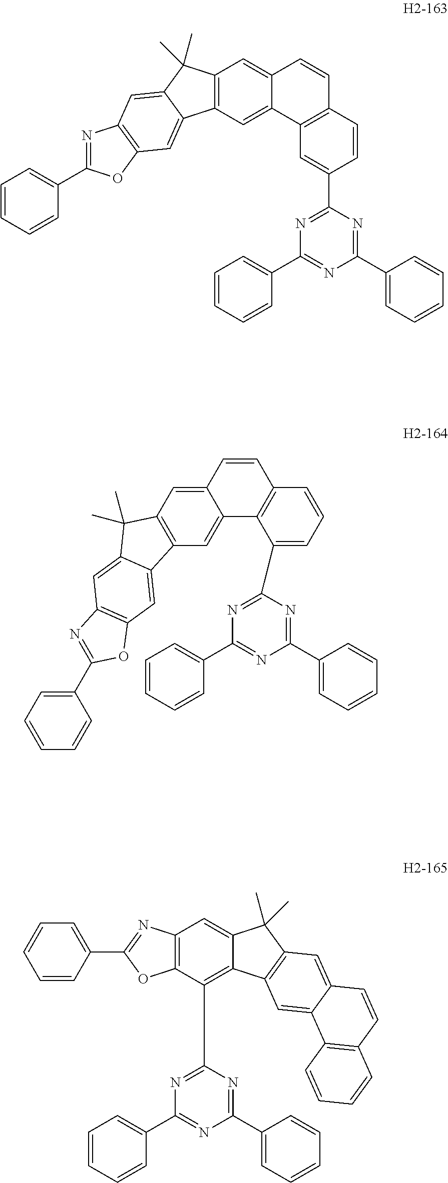 [SMILES: CC1(C)c2cc3ccc4ccc(-c5nc(-c6ccccc6)nc(-c6ccccc6)n5)cc4c3cc2-c2cc3oc(-c4ccccc4)nc3cc21 ; CC1(C)c2cc3ccc4cccc(-c5nc(-c6ccccc6)nc(-c6ccccc6)n5)c4c3cc2-c2cc3oc(-c4ccccc4)nc3cc21 ; CC1(C)c2cc3ccc4ccccc4c3cc2-c2c1cc1nc(-c3ccccc3)oc1c2-c1nc(-c2ccccc2)nc(-c2ccccc2)n1]

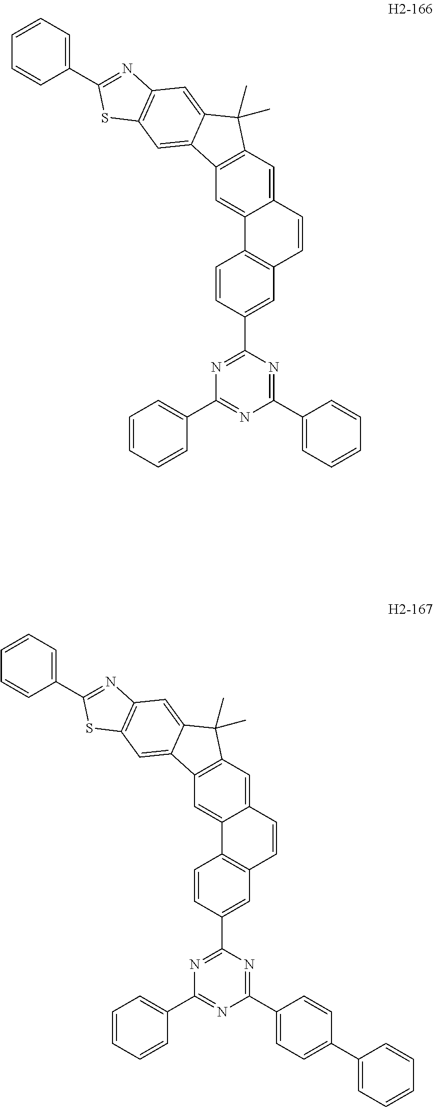 [SMILES: CC1(C)c2cc3ccc4cc(-c5nc(-c6ccccc6)nc(-c6ccccc6)n5)ccc4c3cc2-c2cc3sc(-c4ccccc4)nc3cc21 ; CC1(C)c2cc3ccc4cc(-c5nc(-c6ccccc6)nc(-c6ccc(-c7ccccc7)cc6)n5)ccc4c3cc2-c2cc3sc(-c4ccccc4)nc3cc21]

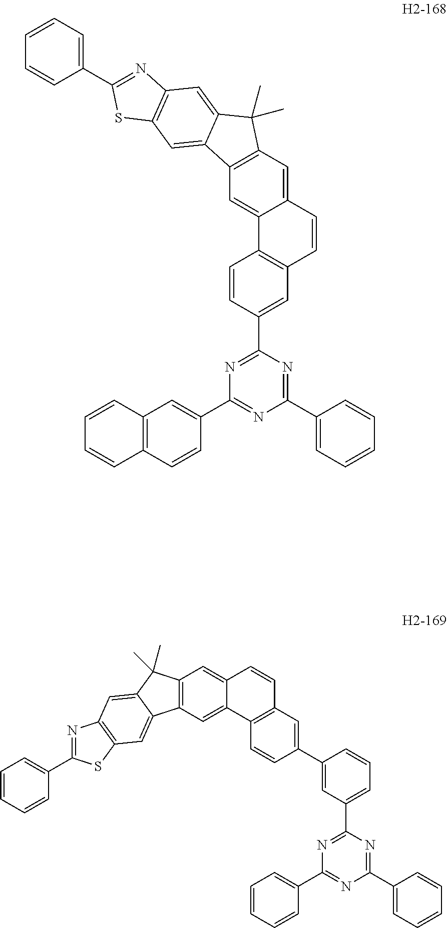 [SMILES: CC1(C)c2cc3ccc4cc(-c5nc(-c6ccccc6)nc(-c6ccc7ccccc7c6)n5)ccc4c3cc2-c2cc3sc(-c4ccccc4)nc3cc21 ; CC1(C)c2cc3ccc4cc(-c5cccc(-c6nc(-c7ccccc7)nc(-c7ccccc7)n6)c5)ccc4c3cc2-c2cc3sc(-c4ccccc4)nc3cc21]

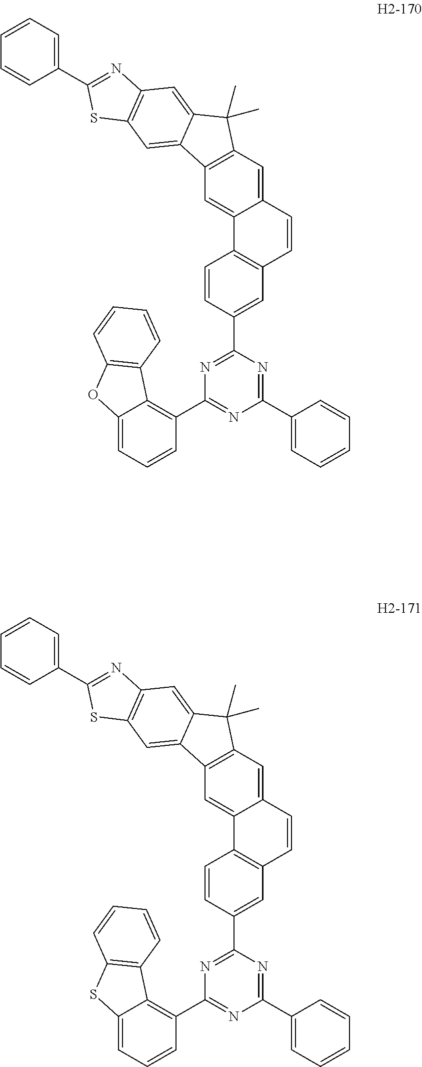 [SMILES: CC1(C)c2cc3ccc4cc(-c5nc(-c6ccccc6)nc(-c6cccc7oc8ccccc8c67)n5)ccc4c3cc2-c2cc3sc(-c4ccccc4)nc3cc21 ; CC1(C)c2cc3ccc4cc(-c5nc(-c6ccccc6)nc(-c6cccc7sc8ccccc8c67)n5)ccc4c3cc2-c2cc3sc(-c4ccccc4)nc3cc21]

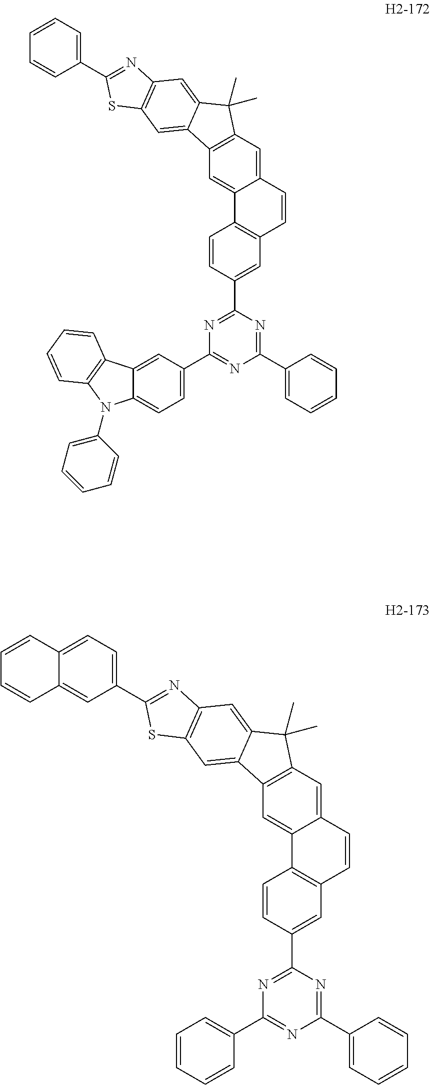 [SMILES: CC1(C)c2cc3ccc4cc(-c5nc(-c6ccccc6)nc(-c6ccc7c(c6)c6ccccc6n7-c6ccccc6)n5)ccc4c3cc2-c2cc3sc(-c4ccccc4)nc3cc21 ; CC1(C)c2cc3ccc4cc(-c5nc(-c6ccccc6)nc(-c6ccccc6)n5)ccc4c3cc2-c2cc3sc(-c4ccc5ccccc5c4)nc3cc21]

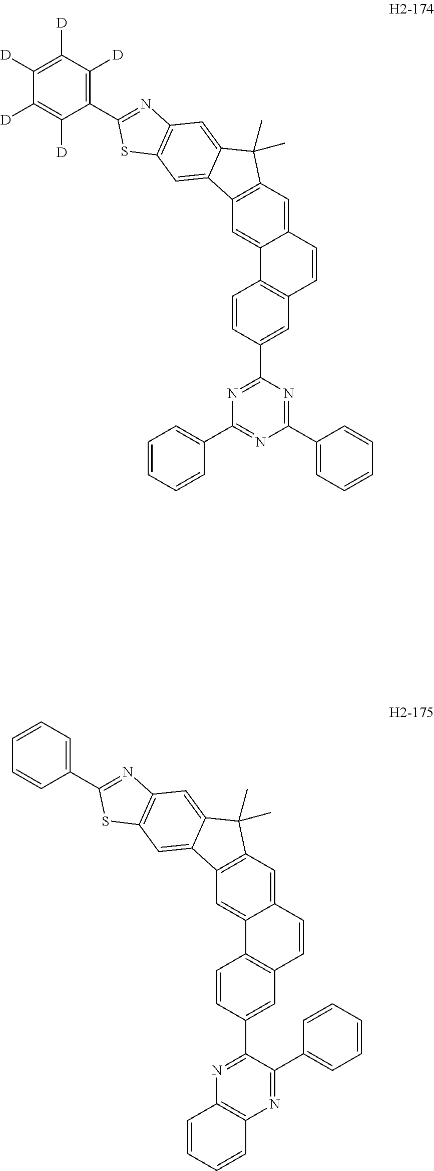 [SMILES: [2H]c1c([2H])c([2H])c(-c2nc3cc4c(cc3s2)-c2cc3c(ccc5cc(-c6nc(-c7ccccc7)nc(-c7ccccc7)n6)ccc53)cc2C4(C)C)c([2H])c1[2H] ; CC1(C)c2cc3ccc4cc(-c5nc6ccccc6nc5-c5ccccc5)ccc4c3cc2-c2cc3sc(-c4ccccc4)nc3cc21]

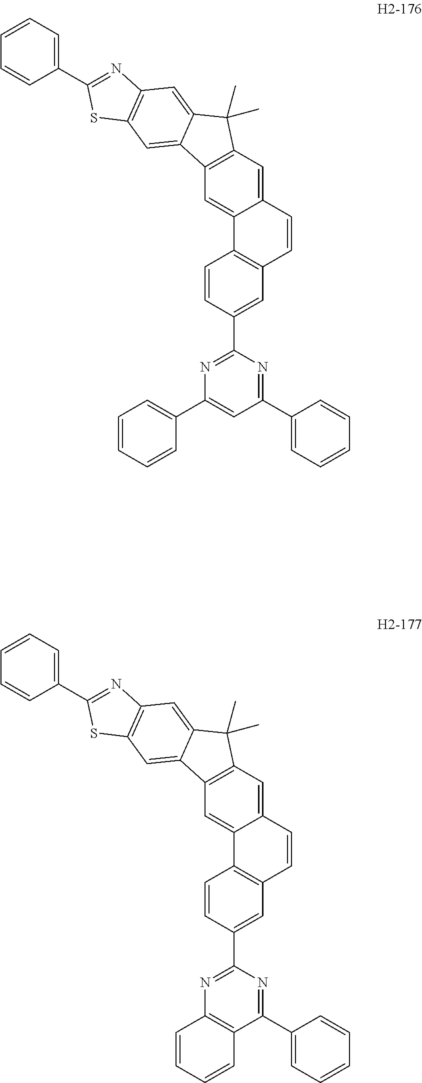 [SMILES: CC1(C)c2cc3ccc4cc(-c5nc(-c6ccccc6)cc(-c6ccccc6)n5)ccc4c3cc2-c2cc3sc(-c4ccccc4)nc3cc21 ; CC1(C)c2cc3ccc4cc(-c5nc(-c6ccccc6)c6ccccc6n5)ccc4c3cc2-c2cc3sc(-c4ccccc4)nc3cc21]

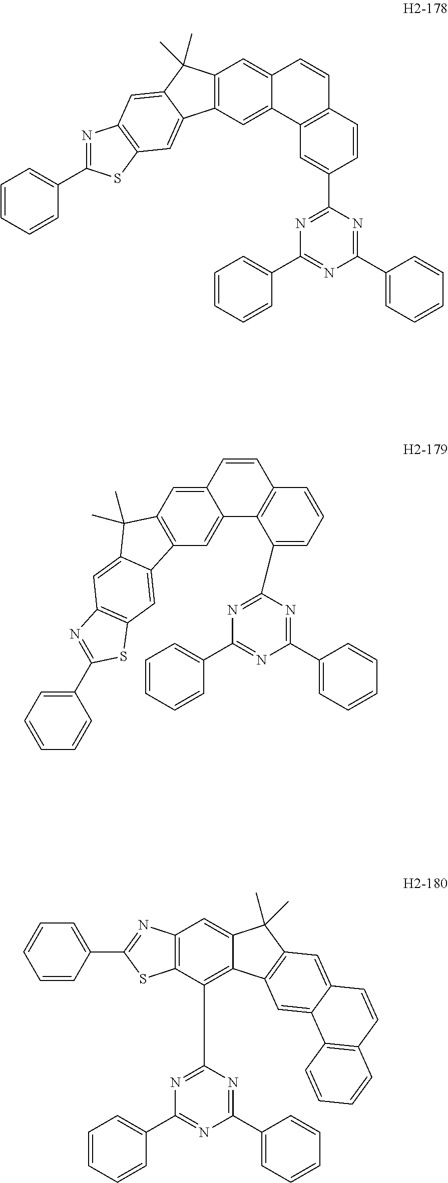 [SMILES: CC1(C)c2cc3ccc4ccc(-c5nc(-c6ccccc6)nc(-c6ccccc6)n5)cc4c3cc2-c2cc3sc(-c4ccccc4)nc3cc21 ; CC1(C)c2cc3ccc4cccc(-c5nc(-c6ccccc6)nc(-c6ccccc6)n5)c4c3cc2-c2cc3sc(-c4ccccc4)nc3cc21 ; CC1(C)c2cc3ccc4ccccc4c3cc2-c2c1cc1nc(-c3ccccc3)sc1c2-c1nc(-c2ccccc2)nc(-c2ccccc2)n1]

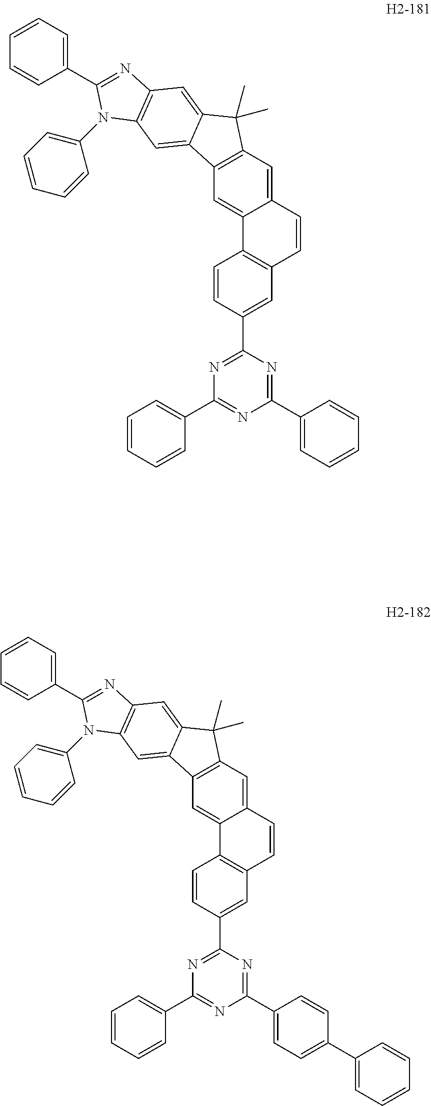 [SMILES: CC1(C)c2cc3ccc4cc(-c5nc(-c6ccccc6)nc(-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccc(-c7ccccc7)cc6)n5)ccc4c3cc2-c2cc3c(cc21)nc(-c1ccccc1)n3-c1ccccc1]

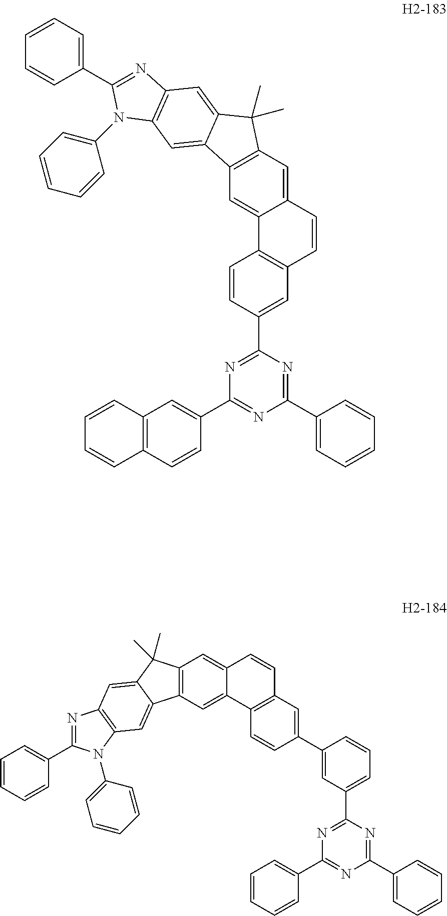 [SMILES: CC1(C)c2cc3ccc4cc(-c5nc(-c6ccccc6)nc(-c6ccc7ccccc7c6)n5)ccc4c3cc2-c2cc3c(cc21)nc(-c1ccccc1)n3-c1ccccc1 ; CC1(C)c2cc3ccc4cc(-c5cccc(-c6nc(-c7ccccc7)nc(-c7ccccc7)n6)c5)ccc4c3cc2-c2cc3c(cc21)nc(-c1ccccc1)n3-c1ccccc1]

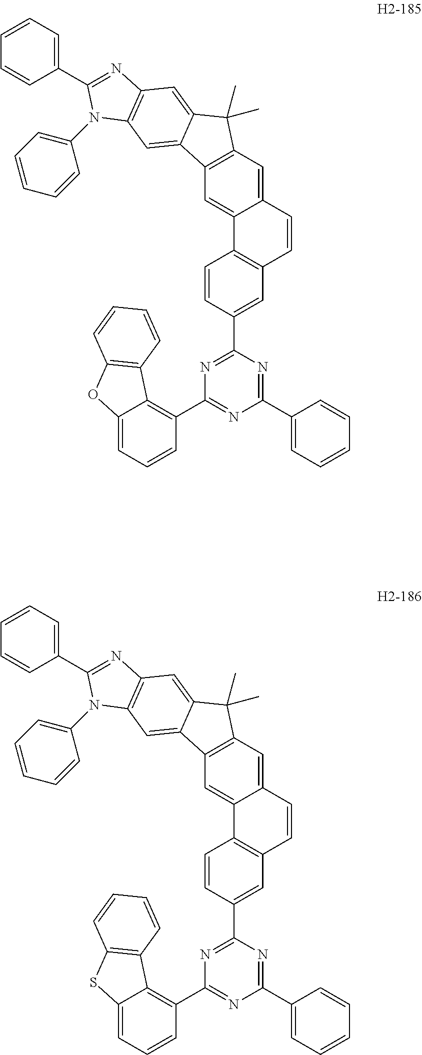 [SMILES: CC1(C)c2cc3ccc4cc(-c5nc(-c6ccccc6)nc(-c6cccc7oc8ccccc8c67)n5)ccc4c3cc2-c2cc3c(cc21)nc(-c1ccccc1)n3-c1ccccc1 ; CC1(C)c2cc3ccc4cc(-c5nc(-c6ccccc6)nc(-c6cccc7sc8ccccc8c67)n5)ccc4c3cc2-c2cc3c(cc21)nc(-c1ccccc1)n3-c1ccccc1]

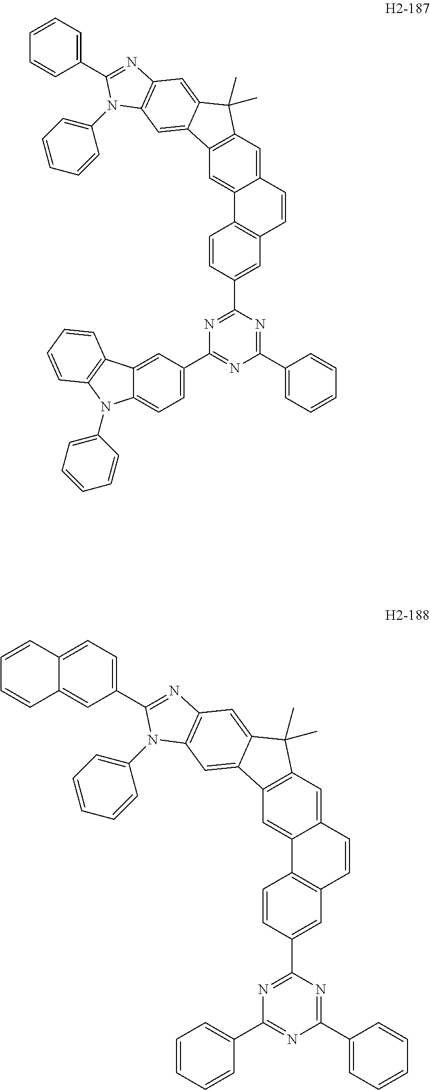 [SMILES: CC1(C)c2cc3ccc4cc(-c5nc(-c6ccccc6)nc(-c6ccc7c(c6)c6ccccc6n7-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccccc6)n5)ccc4c3cc2-c2cc3c(cc21)nc(-c1ccc2ccccc2c1)n3-c1ccccc1]

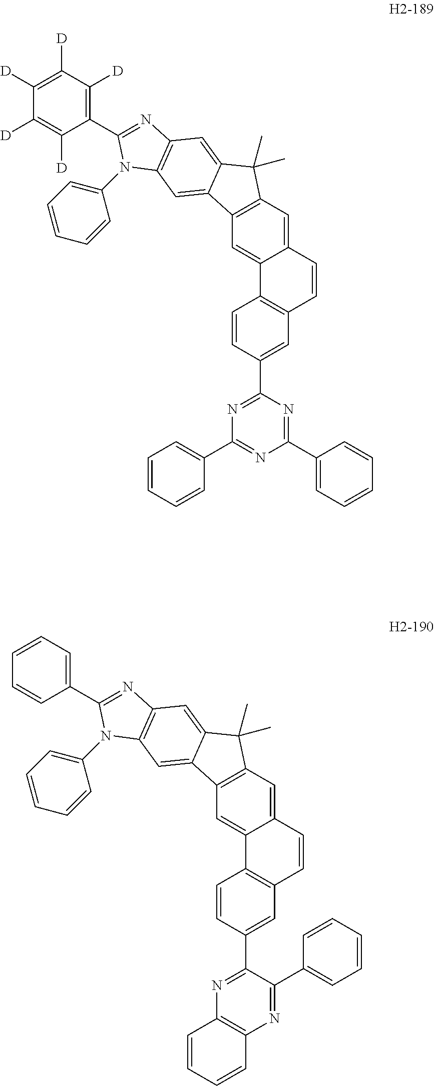 [SMILES: [2H]c1c([2H])c([2H])c(-c2nc3cc4c(cc3n2-c2ccccc2)-c2cc3c(ccc5cc(-c6nc(-c7ccccc7)nc(-c7ccccc7)n6)ccc53)cc2C4(C)C)c([2H])c1[2H] ; CC1(C)c2cc3ccc4cc(-c5nc6ccccc6nc5-c5ccccc5)ccc4c3cc2-c2cc3c(cc21)nc(-c1ccccc1)n3-c1ccccc1]

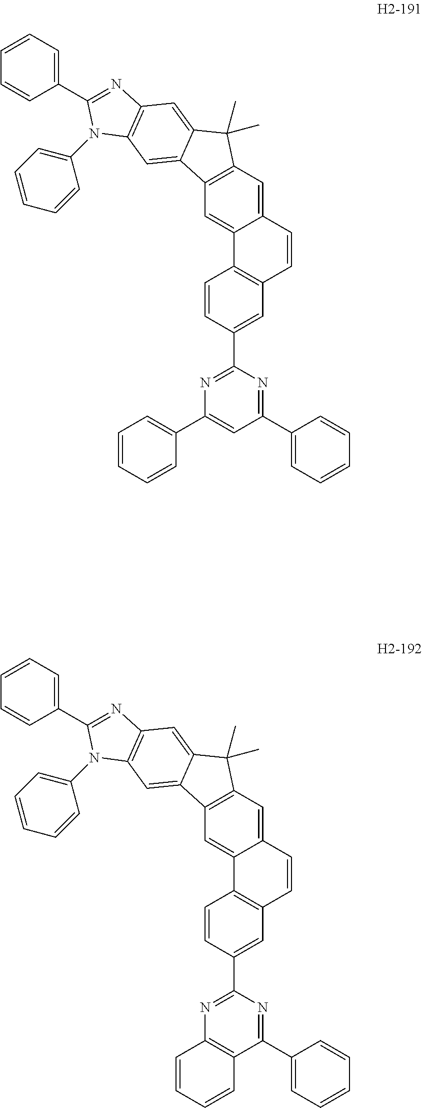 [SMILES: CC1(C)c2cc3ccc4cc(-c5nc(-c6ccccc6)cc(-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)c6ccccc6n5)ccc4c3cc2-c2cc3c(cc21)nc(-c1ccccc1)n3-c1ccccc1]

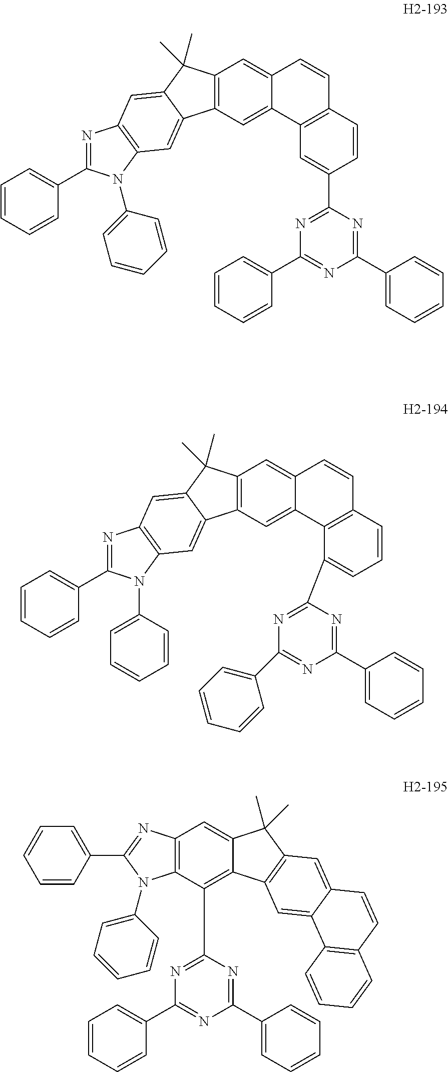 [SMILES: CC1(C)c2cc3ccc4ccc(-c5nc(-c6ccccc6)nc(-c6ccccc6)n5)cc4c3cc2-c2cc3c(cc21)nc(-c1ccccc1)n3-c1ccccc1 ; CC1(C)c2cc3ccc4cccc(-c5nc(-c6ccccc6)nc(-c6ccccc6)n5)c4c3cc2-c2cc3c(cc21)nc(-c1ccccc1)n3-c1ccccc1 ; CC1(C)c2cc3ccc4ccccc4c3cc2-c2c1cc1nc(-c3ccccc3)n(-c3ccccc3)c1c2-c1nc(-c2ccccc2)nc(-c2ccccc2)n1]

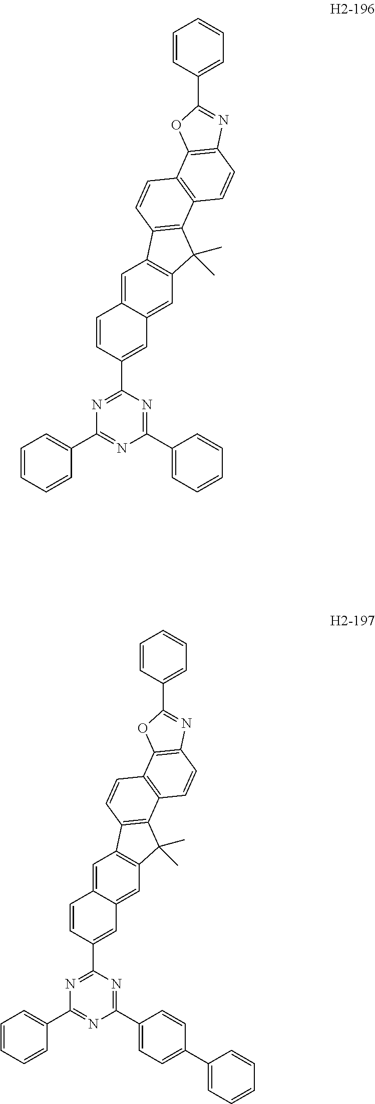 [SMILES: CC1(C)c2cc3cc(-c4nc(-c5ccccc5)nc(-c5ccccc5)n4)ccc3cc2-c2ccc3c(ccc4nc(-c5ccccc5)oc43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)oc43)c21]

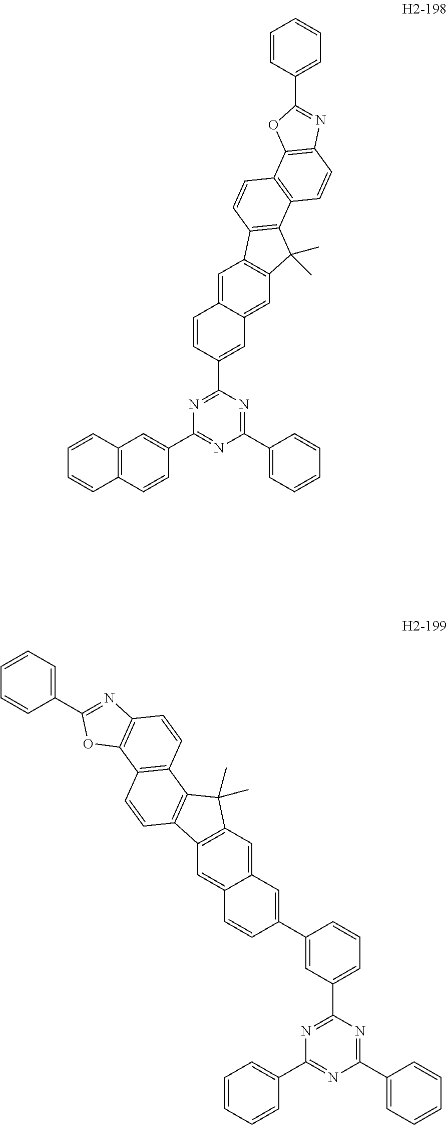 [SMILES: CC1(C)c2cc3cc(-c4nc(-c5ccccc5)nc(-c5ccc6ccccc6c5)n4)ccc3cc2-c2ccc3c(ccc4nc(-c5ccccc5)oc43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)oc43)c21]

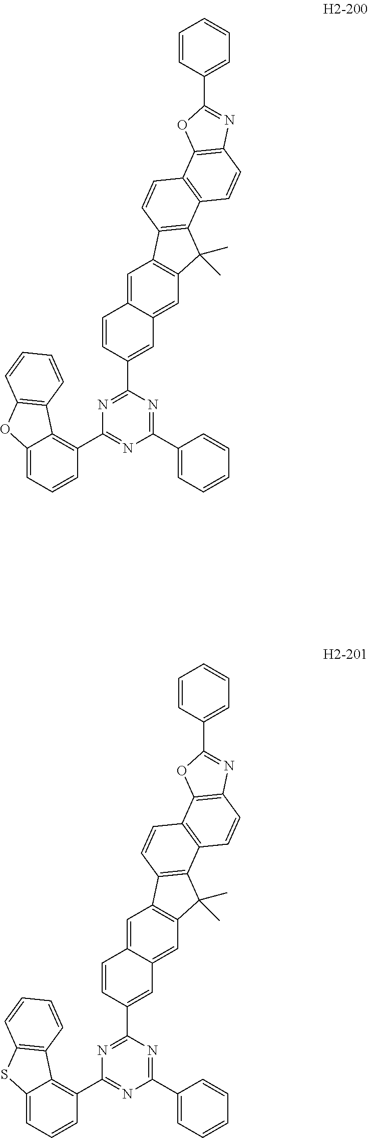 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)oc43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)oc43)c21]

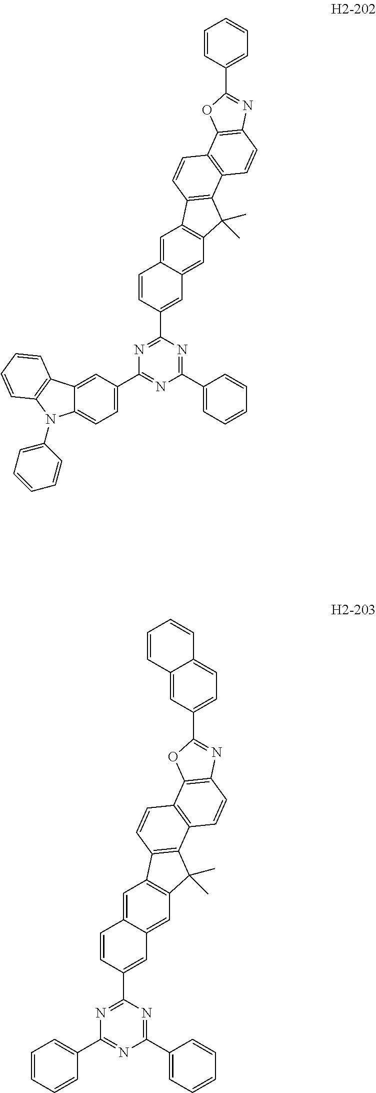 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)oc43)c21 ; CC1(C)c2cc3cc(-c4nc(-c5ccccc5)nc(-c5ccccc5)n4)ccc3cc2-c2ccc3c(ccc4nc(-c5ccc6ccccc6c5)oc43)c21]

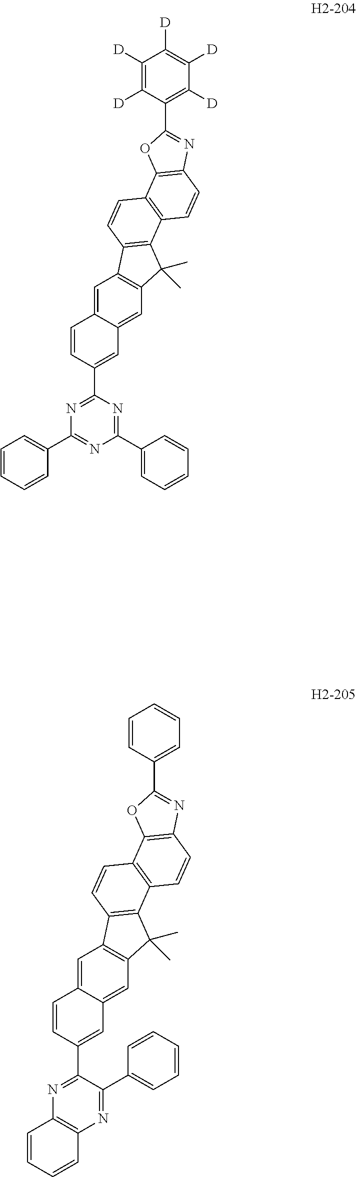 [SMILES: [2H]c1c([2H])c([2H])c(-c2nc3ccc4c5c(ccc4c3o2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)oc43)c21]

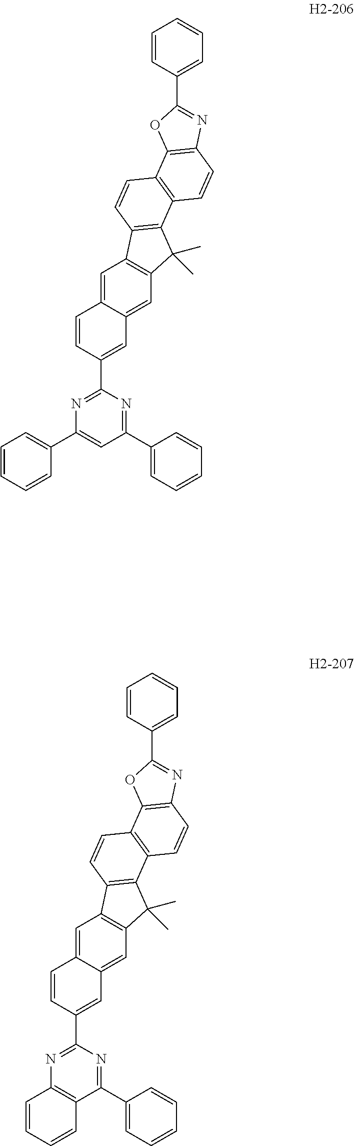 [SMILES: CC1(C)c2cc3cc(-c4nc(-c5ccccc5)cc(-c5ccccc5)n4)ccc3cc2-c2ccc3c(ccc4nc(-c5ccccc5)oc43)c21 ; CC1(C)c2cc3cc(-c4nc(-c5ccccc5)c5ccccc5n4)ccc3cc2-c2ccc3c(ccc4nc(-c5ccccc5)oc43)c21]

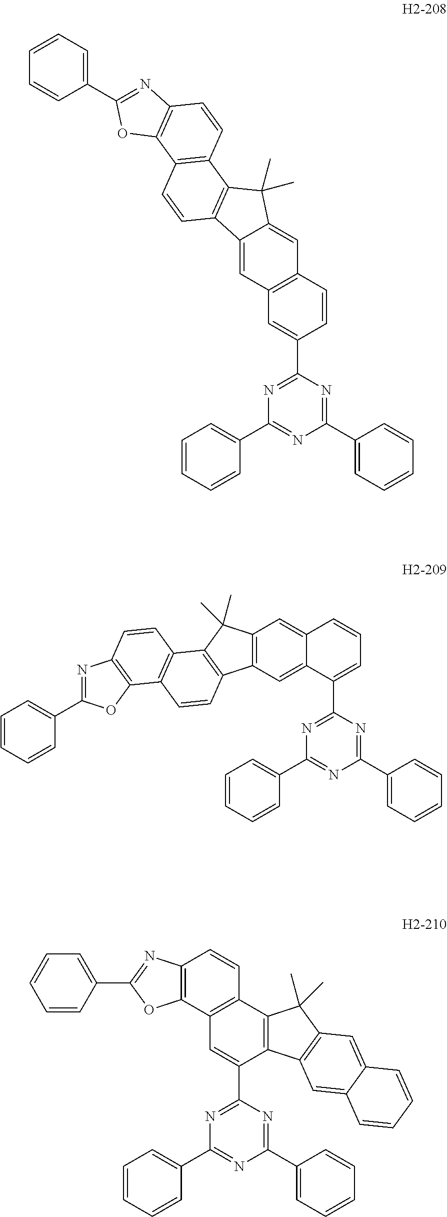 [SMILES: CC1(C)c2cc3ccc(-c4nc(-c5ccccc5)nc(-c5ccccc5)n4)cc3cc2-c2ccc3c(ccc4nc(-c5ccccc5)oc43)c21 ; CC1(C)c2cc3cccc(-c4nc(-c5ccccc5)nc(-c5ccccc5)n4)c3cc2-c2ccc3c(ccc4nc(-c5ccccc5)oc43)c21 ; CC1(C)c2cc3ccccc3cc2-c2c(-c3nc(-c4ccccc4)nc(-c4ccccc4)n3)cc3c(ccc4nc(-c5ccccc5)oc43)c21]

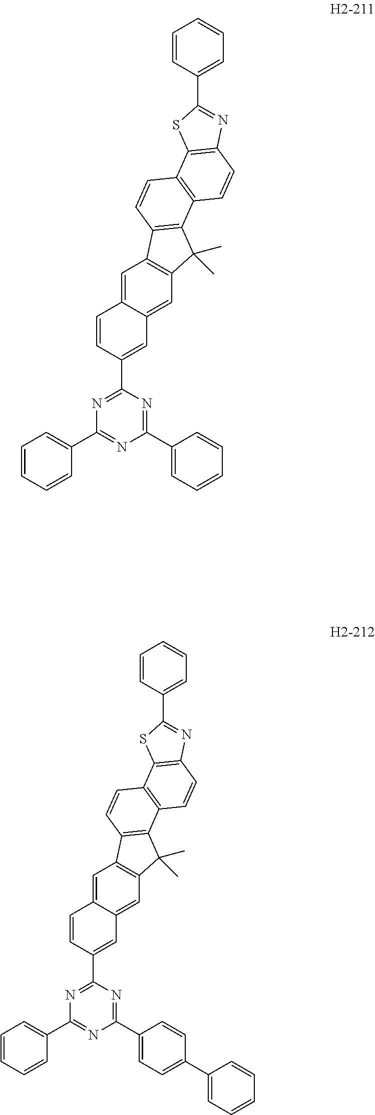 [SMILES: CC1(C)c2cc3cc(-c4nc(-c5ccccc5)nc(-c5ccccc5)n4)ccc3cc2-c2ccc3c(ccc4nc(-c5ccccc5)sc43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)sc43)c21]

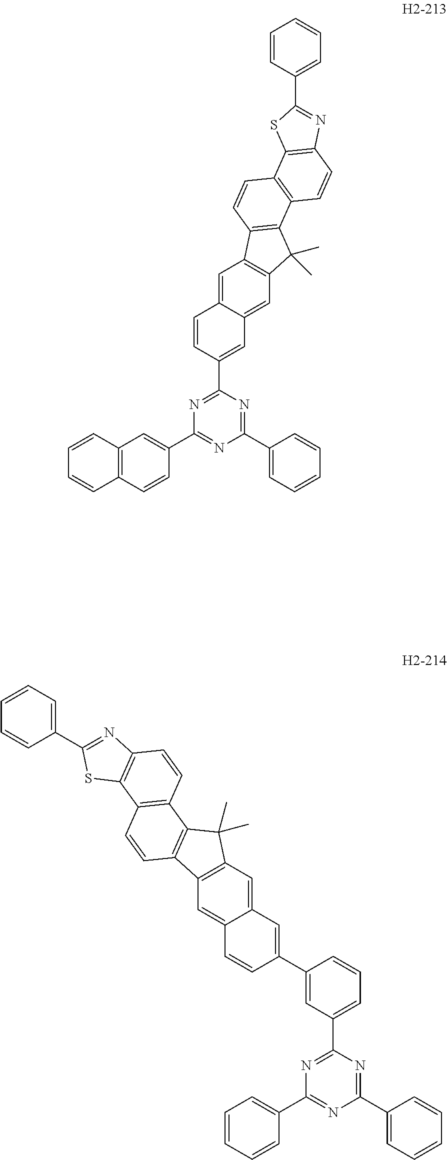 [SMILES: CC1(C)c2cc3cc(-c4nc(-c5ccccc5)nc(-c5ccc6ccccc6c5)n4)ccc3cc2-c2ccc3c(ccc4nc(-c5ccccc5)sc43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)sc43)c21]

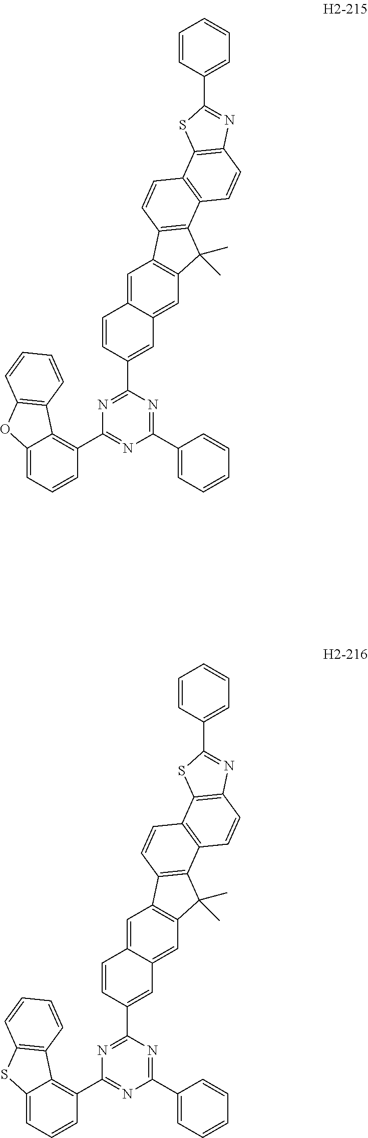 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)sc43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)sc43)c21]

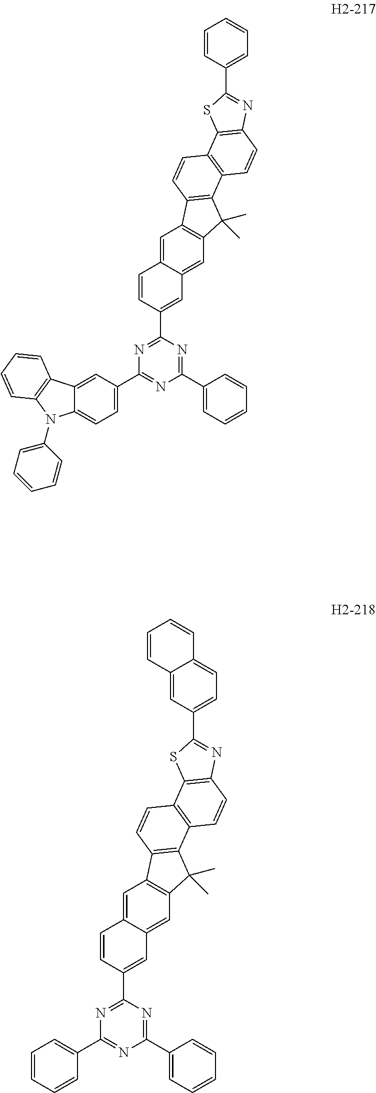 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)sc43)c21 ; CC1(C)c2cc3cc(-c4nc(-c5ccccc5)nc(-c5ccccc5)n4)ccc3cc2-c2ccc3c(ccc4nc(-c5ccc6ccccc6c5)sc43)c21]

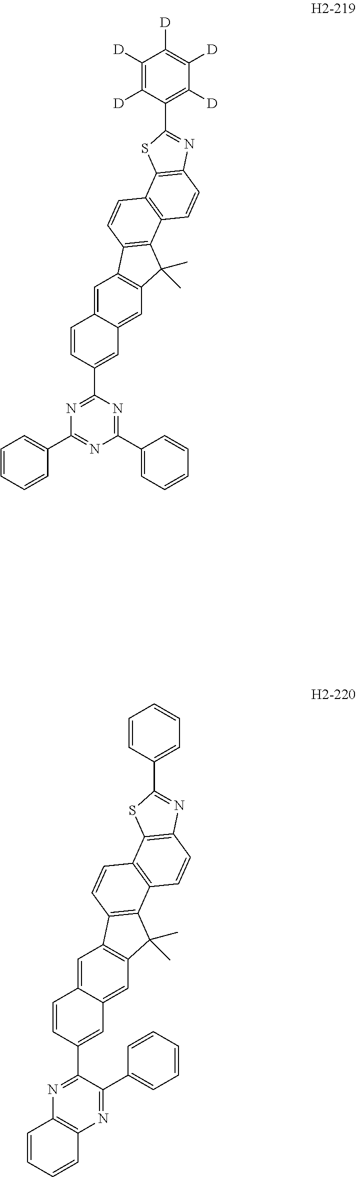 [SMILES: [2H]c1c([2H])c([2H])c(-c2nc3ccc4c5c(ccc4c3s2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)sc43)c21]

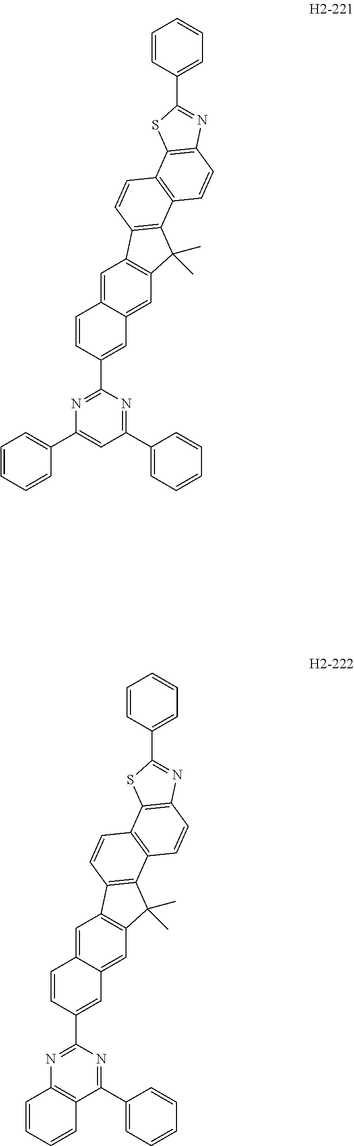 [SMILES: CC1(C)c2cc3cc(-c4nc(-c5ccccc5)cc(-c5ccccc5)n4)ccc3cc2-c2ccc3c(ccc4nc(-c5ccccc5)sc43)c21 ; CC1(C)c2cc3cc(-c4nc(-c5ccccc5)c5ccccc5n4)ccc3cc2-c2ccc3c(ccc4nc(-c5ccccc5)sc43)c21]

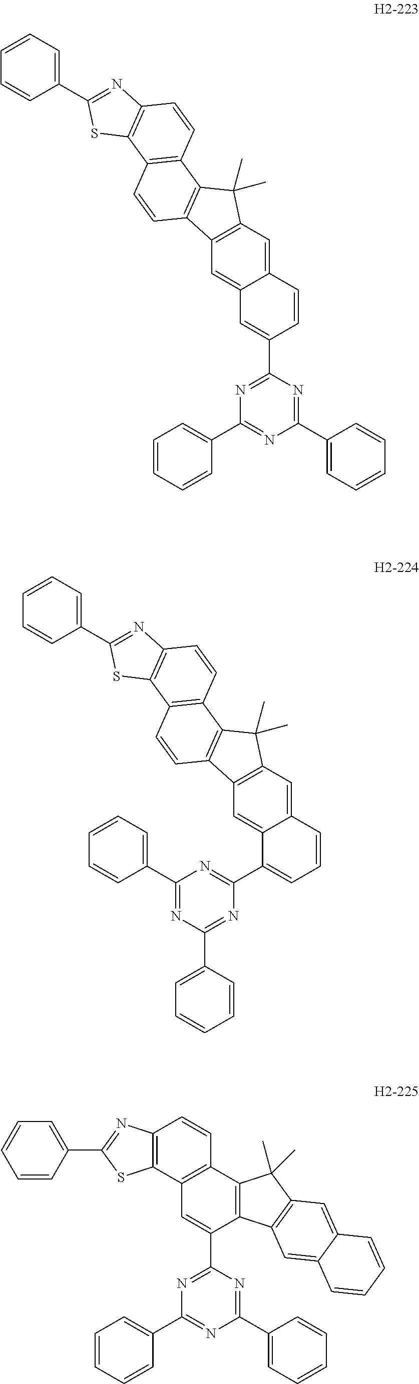 [SMILES: CC1(C)c2cc3ccc(-c4nc(-c5ccccc5)nc(-c5ccccc5)n4)cc3cc2-c2ccc3c(ccc4nc(-c5ccccc5)sc43)c21 ; CC1(C)c2cc3cccc(-c4nc(-c5ccccc5)nc(-c5ccccc5)n4)c3cc2-c2ccc3c(ccc4nc(-c5ccccc5)sc43)c21 ; CC1(C)c2cc3ccccc3cc2-c2c(-c3nc(-c4ccccc4)nc(-c4ccccc4)n3)cc3c(ccc4nc(-c5ccccc5)sc43)c21]

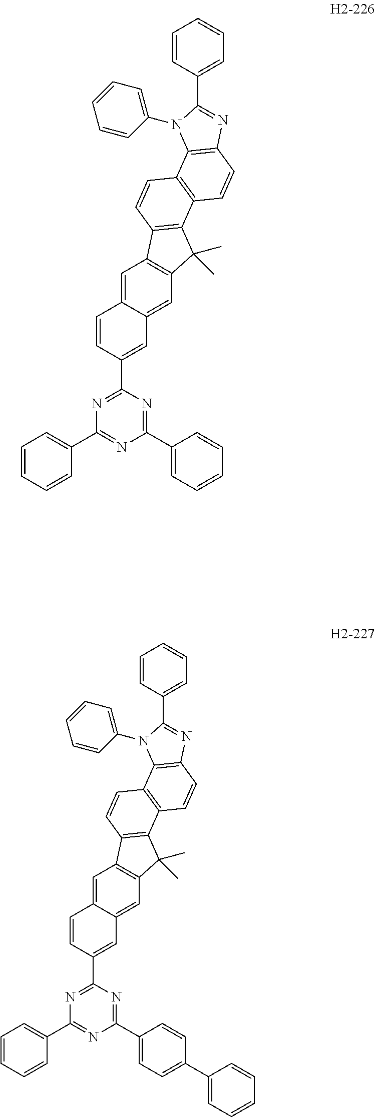 [SMILES: CC1(C)c2cc3cc(-c4nc(-c5ccccc5)nc(-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)n(-c5ccccc5)c43)c21]

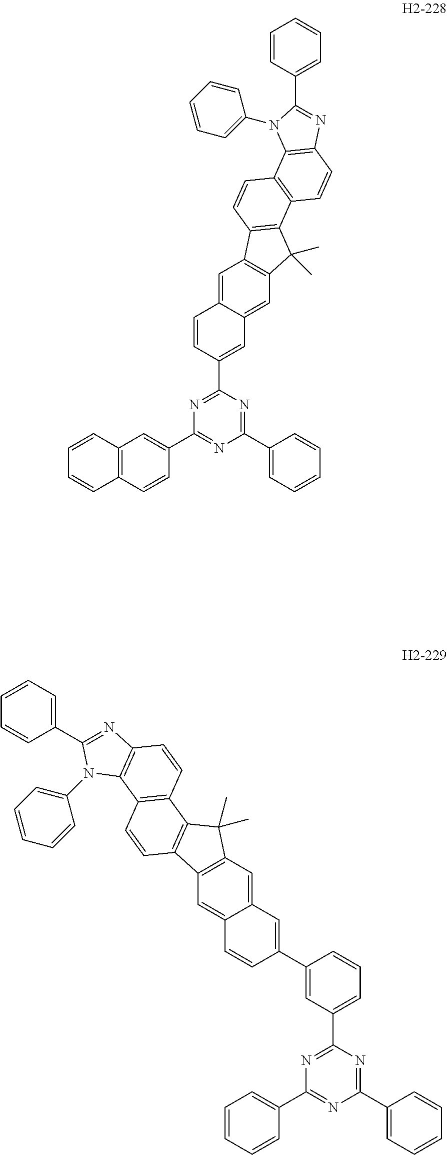 [SMILES: CC1(C)c2cc3cc(-c4nc(-c5ccccc5)nc(-c5ccc6ccccc6c5)n4)ccc3cc2-c2ccc3c(ccc4nc(-c5ccccc5)n(-c5ccccc5)c43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)n(-c5ccccc5)c43)c21]

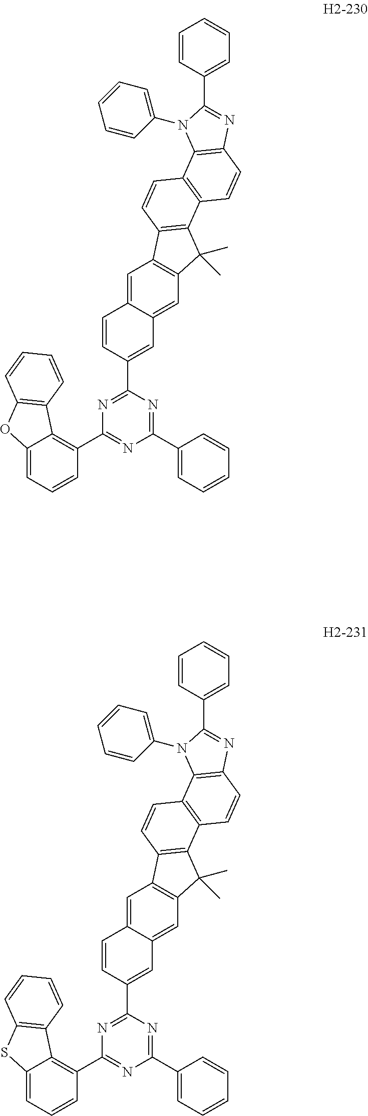 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)n(-c5ccccc5)c43)c21]

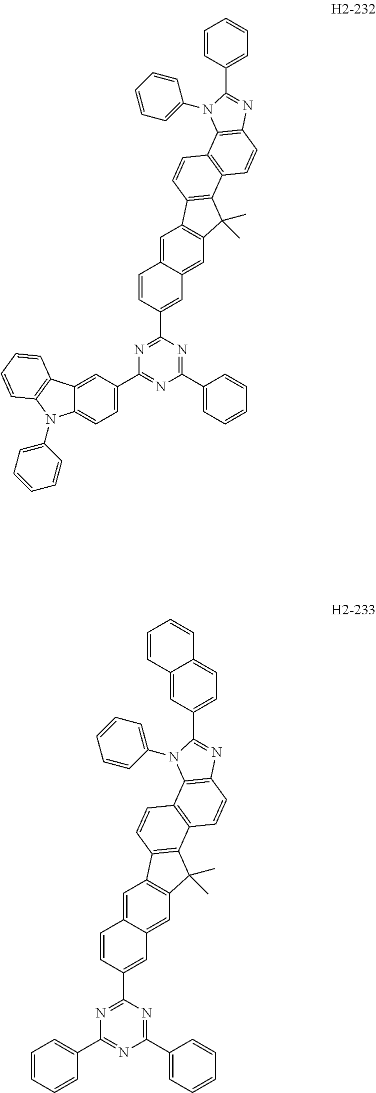 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccccc5)n4)ccc3cc2-c2ccc3c(ccc4nc(-c5ccc6ccccc6c5)n(-c5ccccc5)c43)c21]

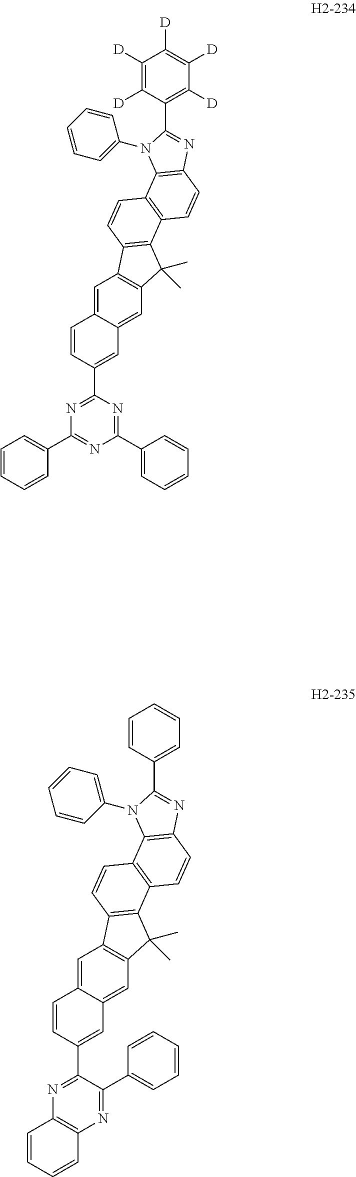 [SMILES: [2H]c1c([2H])c([2H])c(-c2nc3ccc4c5c(ccc4c3n2-c2ccccc2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)n(-c5ccccc5)c43)c21]

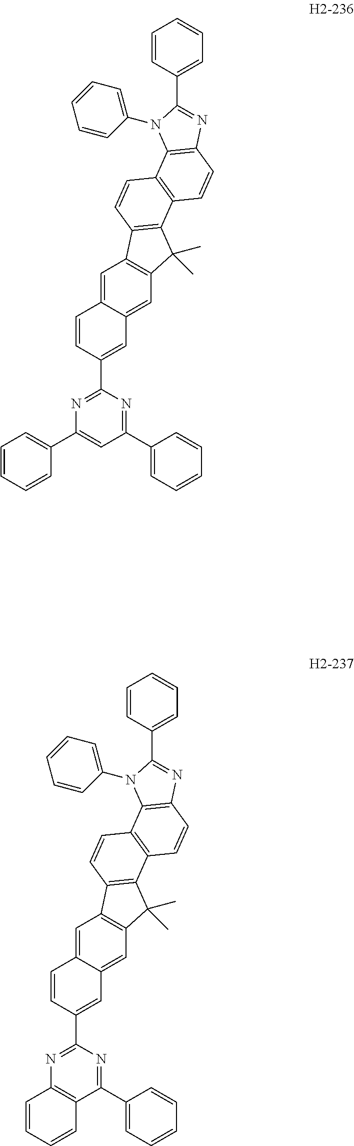 [SMILES: CC1(C)c2cc3cc(-c4nc(-c5ccccc5)cc(-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)c5ccccc5n4)ccc3cc2-c2ccc3c(ccc4nc(-c5ccccc5)n(-c5ccccc5)c43)c21]

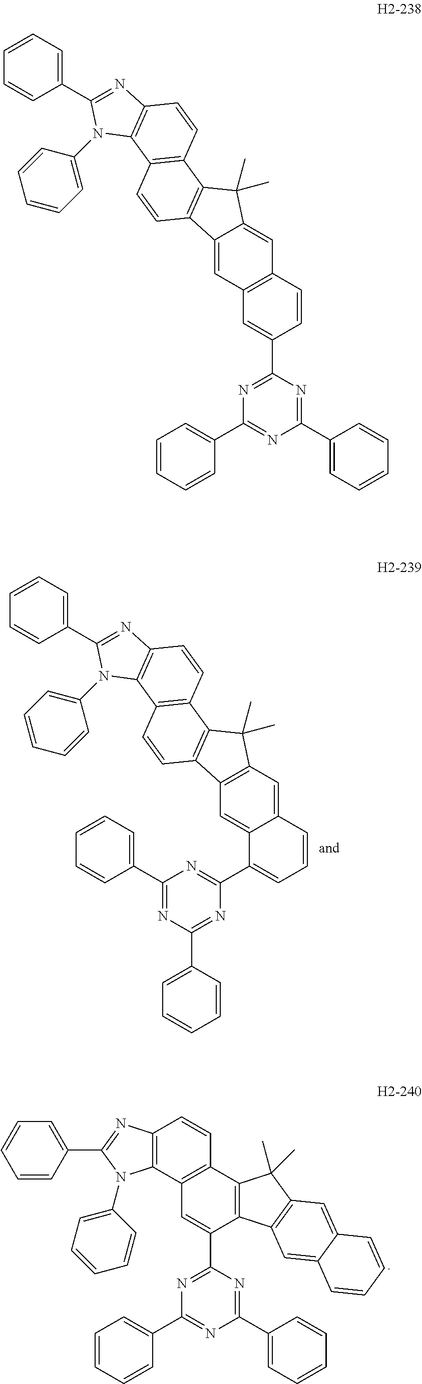 [SMILES: CC1(C)c2cc3ccc(-c4nc(-c5ccccc5)nc(-c5ccccc5)n4)cc3cc2-c2ccc3c(ccc4nc(-c5ccccc5)n(-c5ccccc5)c43)c21 ; CC1(C)c2cc3cccc(-c4nc(-c5ccccc5)nc(-c5ccccc5)n4)c3cc2-c2ccc3c(ccc4nc(-c5ccccc5)n(-c5ccccc5)c43)c21 ; CC1(C)c2cc3ccccc3cc2-c2c(-c3nc(-c4ccccc4)nc(-c4ccccc4)n3)cc3c(ccc4nc(-c5ccccc5)n(-c5ccccc5)c43)c21]

9. An organic electroluminescent device comprising a first electrode; a second electrode; and at least one light-emitting layer between the first electrode and the second electrode, wherein the at least one light-emitting layer comprise a plurality of host materials according to claim 1.

10. An organic electroluminescent compound represented by the following Formula 2′: 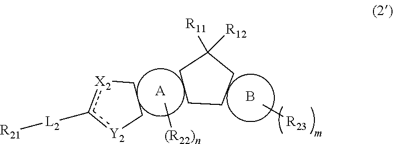
wherein,
ring A and ring B each independently represent a substituted or unsubstituted (C₆-C₃₀)arene or a substituted or unsubstituted (3- to 30-membered)heteroarene;
R₁₁ and R₁₂ each independently represent a substituted or unsubstituted (C₁-C₃₀)alkyl, a substituted or unsubstituted (C₆-C₃₀)aryl, or a substituted or unsubstituted (3-to 30-membered)heteroaryl; or may be linked to each other to form a ring(s);
X₂ and Y₂ each independently represent —N=, —NR₂₄, —O—, or —S—; provided that any one of X₂ and Y₂ is —N=, and the other of X₂ and Y₂ is —NR₂₄—, —O—, or —S—;
R₂₁ and R₂₄ each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and a (C₆-C₃₀) aromatic ring, or —L₃-HAr;
provided that at least one of R₂₁ to R₂₄ is —L₃-HAr, and if R₂₃ is —L₃-HAr, the case where ring A and ring B are both a substituted or unsubstituted benzene is excluded;
L₂ and L₃ each independently represent a single bond, a substituted or unsubstituted (C₆-C₃₀)arylene, or a substituted or unsubstituted (3- to 30-membered)heteroarylene;
HAr represents a substituted or unsubstituted (3- to 30-membered)heteroaryl containing at least one nitrogen atom; and
n represents an integer of 1 to 4, m represents an integer of 1 to 8, and when n are m are an integer of 2 or more, each of R₂₂ and R₂₃ may be the same or different.

11. The organic electroluminescent compound according to claim 10, wherein HAr in Formula 2 is represented by the following Formula 3 or 4: 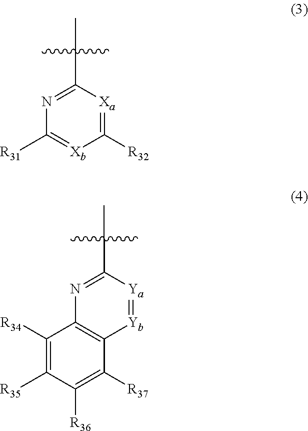
wherein,
Xₐ, X_{b}, Yₐ, and Y_{b} each independently represent N or CR₃₃; provided that any one of Xₐ and X_{b} is N, and at least one of Yₐ and Y_{b} is N; and
R₃₁ to R₃₇ each independently represent hydrogen, deuterium, halogen, cyano, a substituted or unsubstituted (C₁-C₃₀)alkyl, a substituted or unsubstituted (C₆-C₅₀)aryl, a substituted or unsubstituted (3- to 50-membered)heteroaryl, a substituted or unsubstituted (C₃-C₃₀)cycloalkyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted mono- or di(C₁-C₃₀)alkylamino, a substituted or unsubstituted mono- or di(C₆-C₃₀)arylamino, or a substituted or unsubstituted (C₁-C₃₀)alkyl(C₆-C₃₀)arylamino; or may be linked to the adjacent substituents to form a ring(s).

12. The organic electroluminescent compound according to claim 10, wherein Formula 2′ is represented by any one of the following Formulas 2-1a and 2-2 to 2-8: 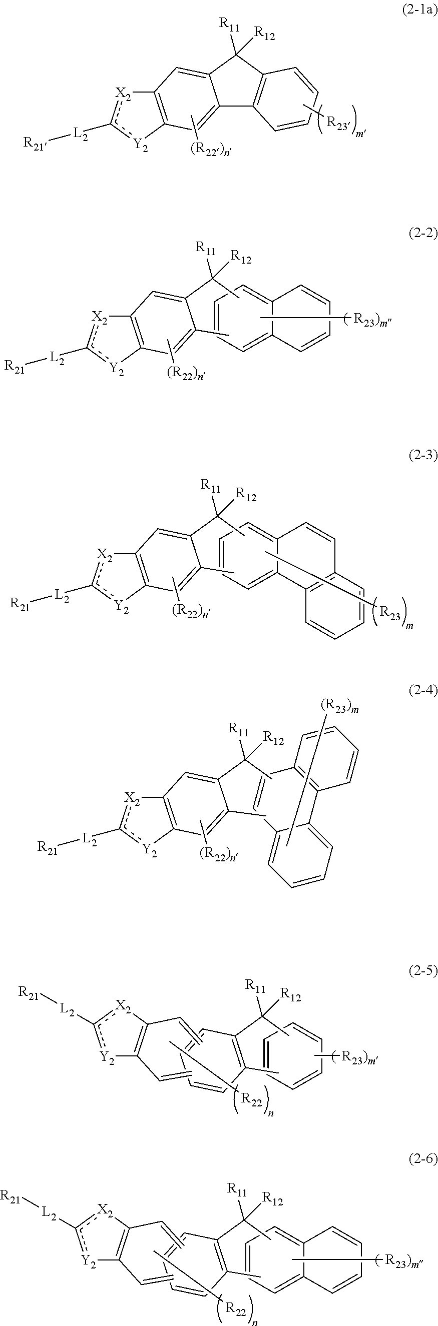 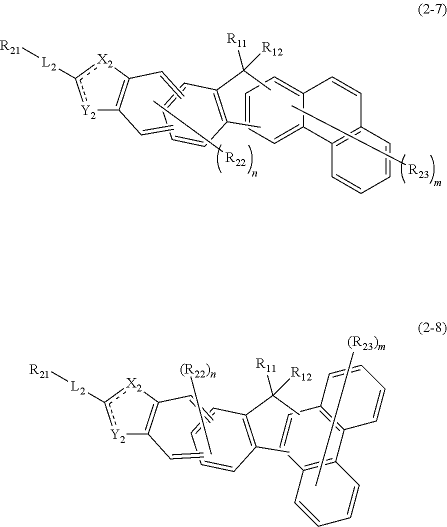
wherein,
R_{21′} and R_{22′} each independently represent hydrogen, deuterium, halogen, cyano, a substituted or unsubstituted (C₁-C₃₀)alkyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀) aliphatic ring and a (C₆-C₃₀) aromatic ring, or —L₃-HAr; provided that at least one of R₂₁ and R_{22′} is —L₃-HAr;
R_{23′} represents hydrogen, deuterium, or (C₆-C₃₀)aryl unsubstituted or unsubstituted with deuterium;
n′ represents an integer of 1 or 2, m′ represents an integer of 1 to 4, and m″ represents an integer of 1 to 6; and
X₂, Y₂, R₁₁, R₁₂, R₂₁ to R₂₃, L₂, n, and m are as defined in claim 10.

13. The organic electroluminescent compound according to claim 10, wherein Formula 2′ is represented by the following Formula 2-9: 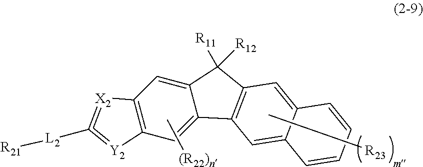
wherein,
X₂, Y₂, R₁₁, R₁₂, R₂₁ to R₂₃, and L₂ are as defined in claim 10; and
n′ represents an integer of 1 or 2, and m″ represents an integer of 1 to 6.

14. The organic electroluminescent compound according to claim 10, wherein the compound represented by Formula 2′ is selected from the following compounds:

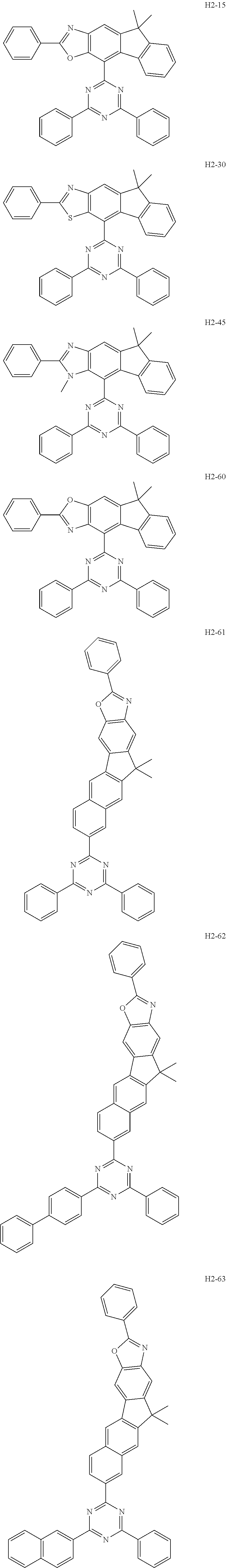

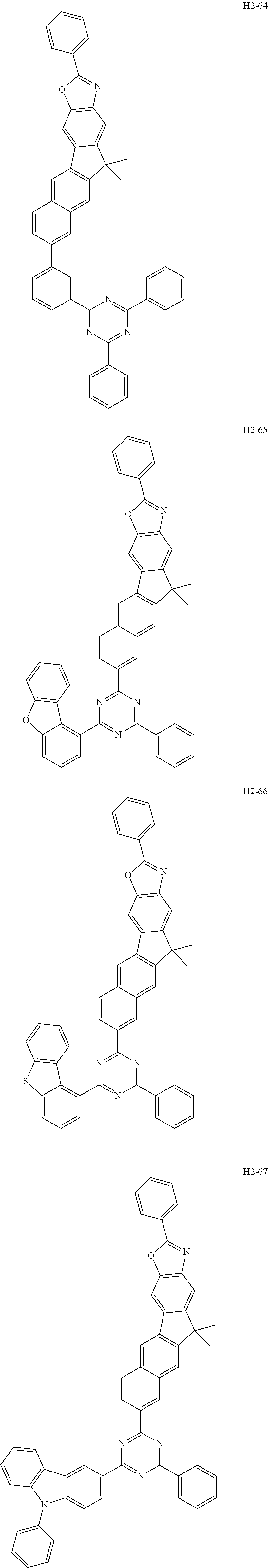 [SMILES: CC1(C)c2cc3cc(-c4cccc(-c5nc(-c6ccccc6)nc(-c6ccccc6)n5)c4)ccc3cc2-c2cc3oc(-c4ccccc4)nc3cc21 ; CC1(C)c2cc3cc(-c4nc(-c5ccccc5)nc(-c5cccc6oc7ccccc7c56)n4)ccc3cc2-c2cc3oc(-c4ccccc4)nc3cc21 ; CC1(C)c2cc3cc(-c4nc(-c5ccccc5)nc(-c5cccc6sc7ccccc7c56)n4)ccc3cc2-c2cc3oc(-c4ccccc4)nc3cc21 ; CC1(C)c2cc3cc(-c4nc(-c5ccccc5)nc(-c5ccc6c(c5)c5ccccc5n6-c5ccccc5)n4)ccc3cc2-c2cc3oc(-c4ccccc4)nc3cc21]

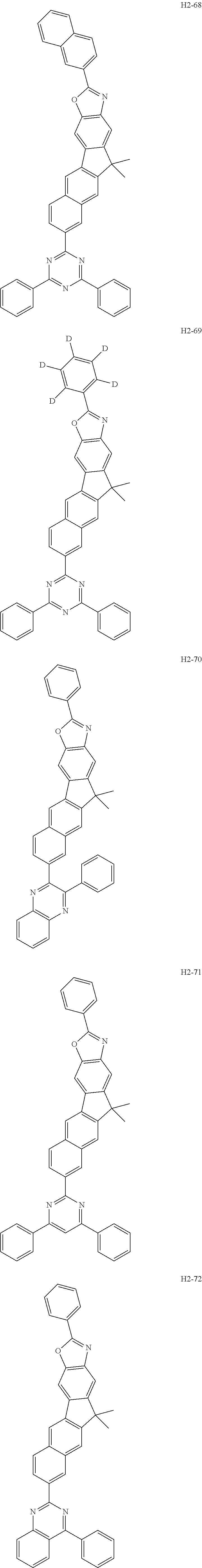 [SMILES: CC1(C)c2cc3cc(-c4nc(-c5ccccc5)nc(-c5ccccc5)n4)ccc3cc2-c2cc3oc(-c4ccc5ccccc5c4)nc3cc21 ; [2H]c1c([2H])c([2H])c(-c2nc3cc4c(cc3o2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3oc(-c4ccccc4)nc3cc21 ; CC1(C)c2cc3cc(-c4nc(-c5ccccc5)cc(-c5ccccc5)n4)ccc3cc2-c2cc3oc(-c4ccccc4)nc3cc21 ; CC1(C)c2cc3cc(-c4nc(-c5ccccc5)c5ccccc5n4)ccc3cc2-c2cc3oc(-c4ccccc4)nc3cc21]

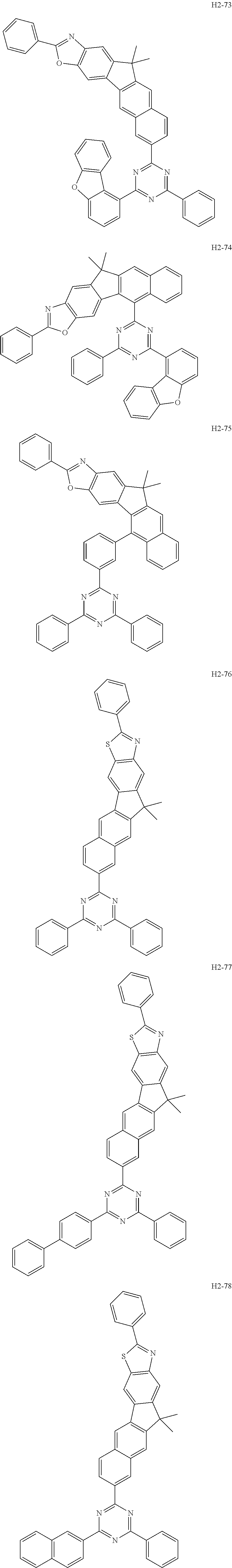

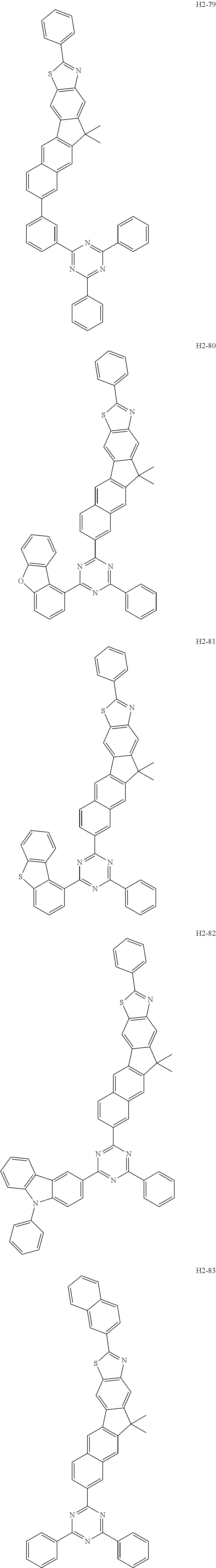 [SMILES: CC1(C)c2cc3cc(-c4cccc(-c5nc(-c6ccccc6)nc(-c6ccccc6)n5)c4)ccc3cc2-c2cc3sc(-c4ccccc4)nc3cc21 ; CC1(C)c2cc3cc(-c4nc(-c5ccccc5)nc(-c5cccc6oc7ccccc7c56)n4)ccc3cc2-c2cc3sc(-c4ccccc4)nc3cc21 ; CC1(C)c2cc3cc(-c4nc(-c5ccccc5)nc(-c5cccc6sc7ccccc7c56)n4)ccc3cc2-c2cc3sc(-c4ccccc4)nc3cc21 ; CC1(C)c2cc3cc(-c4nc(-c5ccccc5)nc(-c5ccc6c(c5)c5ccccc5n6-c5ccccc5)n4)ccc3cc2-c2cc3sc(-c4ccccc4)nc3cc21 ; CC1(C)c2cc3cc(-c4nc(-c5ccccc5)nc(-c5ccccc5)n4)ccc3cc2-c2cc3sc(-c4ccc5ccccc5c4)nc3cc21]

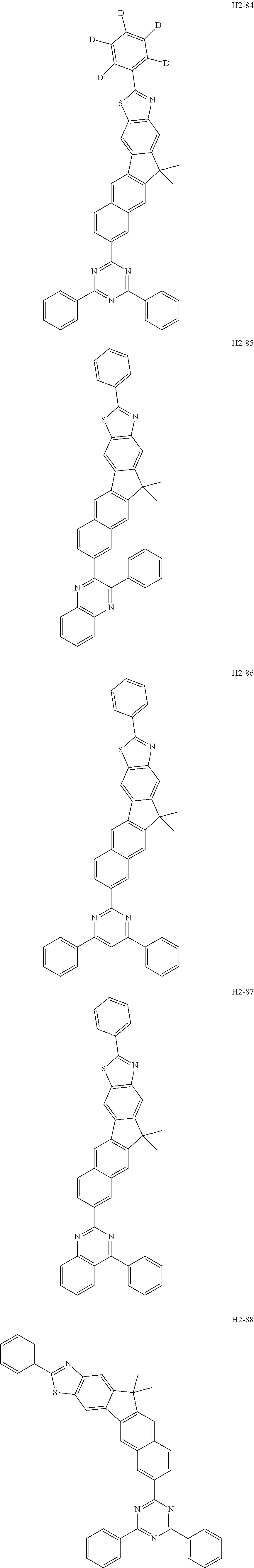 [SMILES: [2H]c1c([2H])c([2H])c(-c2nc3cc4c(cc3s2)-c2cc3ccc(-c5nc(-c6ccccc6)nc(-c6ccccc6)n5)cc3cc2C4(C)C)c([2H])c1[2H] ; CC1(C)c2cc3cc(-c4nc5ccccc5nc4-c4ccccc4)ccc3cc2-c2cc3sc(-c4ccccc4)nc3cc21 ; CC1(C)c2cc3cc(-c4nc(-c5ccccc5)cc(-c5ccccc5)n4)ccc3cc2-c2cc3sc(-c4ccccc4)nc3cc21 ; CC1(C)c2cc3cc(-c4nc(-c5ccccc5)c5ccccc5n4)ccc3cc2-c2cc3sc(-c4ccccc4)nc3cc21 ; CC1(C)c2cc3ccc(-c4nc(-c5ccccc5)nc(-c5ccccc5)n4)cc3cc2-c2cc3sc(-c4ccccc4)nc3cc21]

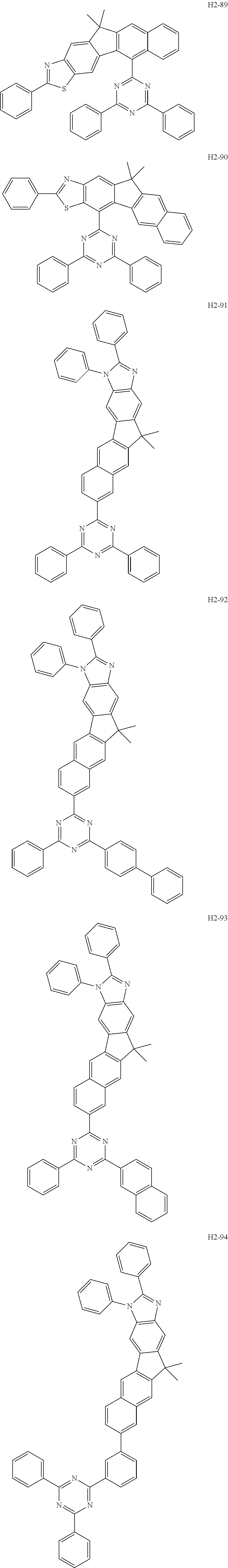

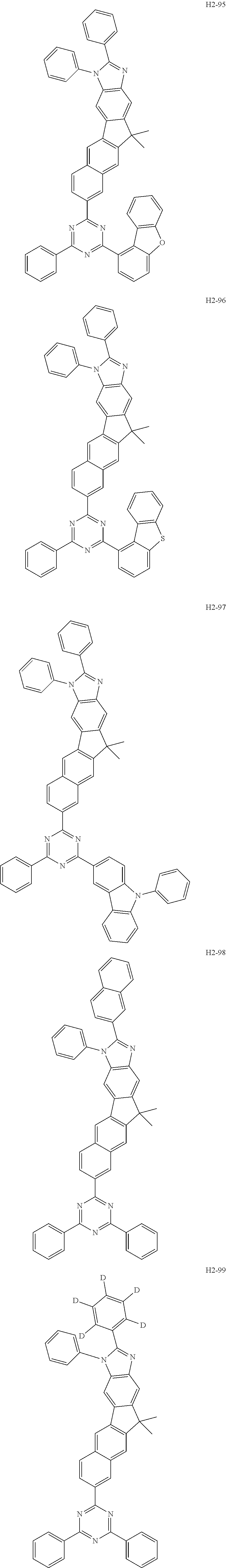

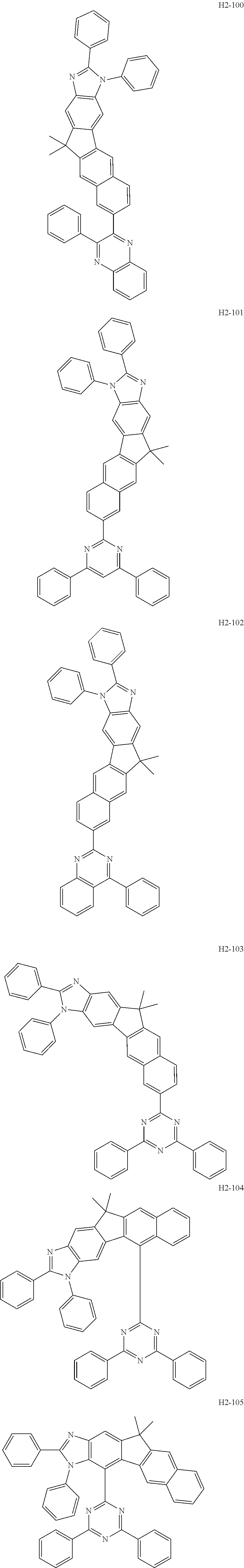

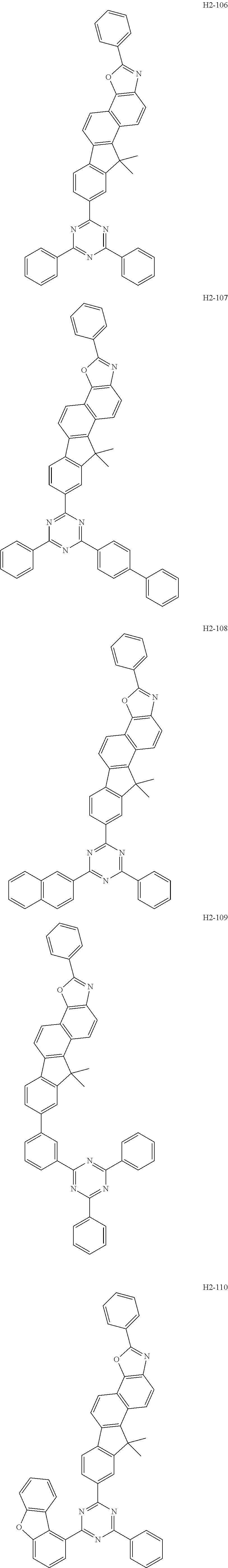

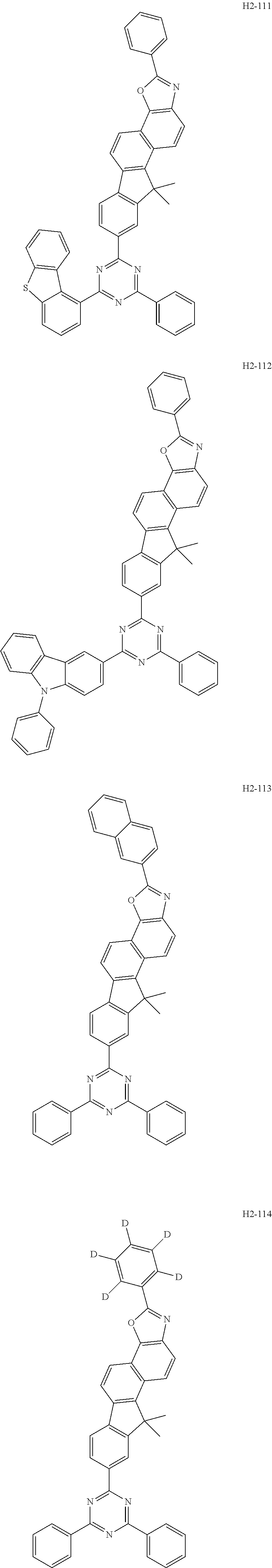 [SMILES: CC1(C)c2cc(-c3nc(-c4ccccc4)nc(-c4cccc5sc6ccccc6c45)n3)ccc2-c2ccc3c(ccc4nc(-c5ccccc5)oc43)c21 ; CC1(C)c2cc(-c3nc(-c4ccccc4)nc(-c4ccc5c(c4)c4ccccc4n5-c4ccccc4)n3)ccc2-c2ccc3c(ccc4nc(-c5ccccc5)oc43)c21 ; CC1(C)c2cc(-c3nc(-c4ccccc4)nc(-c4ccccc4)n3)ccc2-c2ccc3c(ccc4nc(-c5ccc6ccccc6c5)oc43)c21 ; [2H]c1c([2H])c([2H])c(-c2nc3ccc4c5c(ccc4c3o2)-c2ccc(-c3nc(-c4ccccc4)nc(-c4ccccc4)n3)cc2C5(C)C)c([2H])c1[2H]]

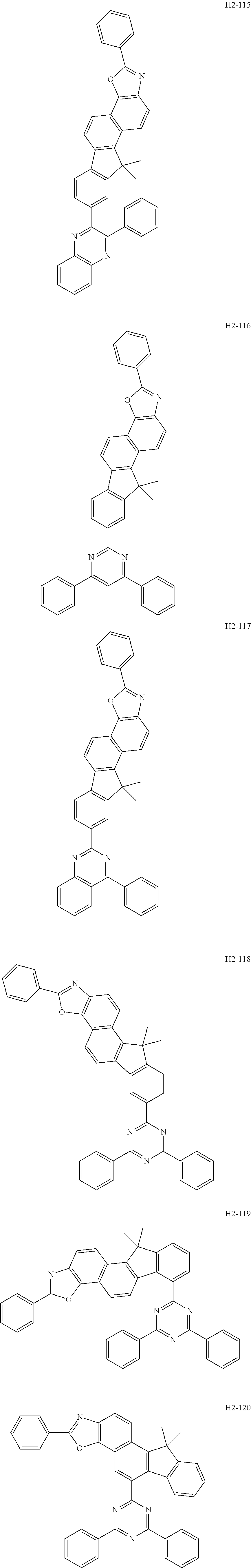 [SMILES: CC1(C)c2cc(-c3nc4ccccc4nc3-c3ccccc3)ccc2-c2ccc3c(ccc4nc(-c5ccccc5)oc43)c21 ; CC1(C)c2cc(-c3nc(-c4ccccc4)cc(-c4ccccc4)n3)ccc2-c2ccc3c(ccc4nc(-c5ccccc5)oc43)c21 ; CC1(C)c2cc(-c3nc(-c4ccccc4)c4ccccc4n3)ccc2-c2ccc3c(ccc4nc(-c5ccccc5)oc43)c21 ; CC1(C)c2ccc(-c3nc(-c4ccccc4)nc(-c4ccccc4)n3)cc2-c2ccc3c(ccc4nc(-c5ccccc5)oc43)c21 ; CC1(C)c2cccc(-c3nc(-c4ccccc4)nc(-c4ccccc4)n3)c2-c2ccc3c(ccc4nc(-c5ccccc5)oc43)c21 ; CC1(C)c2ccccc2-c2c(-c3nc(-c4ccccc4)nc(-c4ccccc4)n3)cc3c(ccc4nc(-c5ccccc5)oc43)c21]

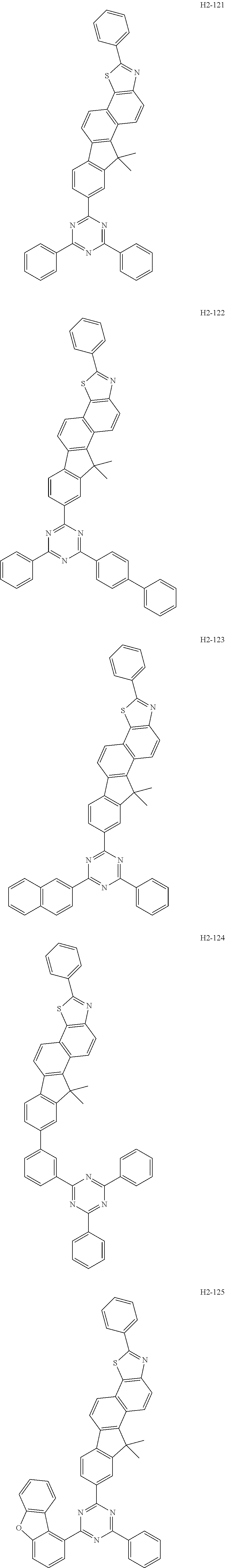 [SMILES: CC1(C)c2cc(-c3nc(-c4ccccc4)nc(-c4ccccc4)n3)ccc2-c2ccc3c(ccc4nc(-c5ccccc5)sc43)c21 ; CC1(C)c2cc(-c3nc(-c4ccccc4)nc(-c4ccc(-c5ccccc5)cc4)n3)ccc2-c2ccc3c(ccc4nc(-c5ccccc5)sc43)c21 ; CC1(C)c2cc(-c3nc(-c4ccccc4)nc(-c4ccc5ccccc5c4)n3)ccc2-c2ccc3c(ccc4nc(-c5ccccc5)sc43)c21 ; CC1(C)c2cc(-c3cccc(-c4nc(-c5ccccc5)nc(-c5ccccc5)n4)c3)ccc2-c2ccc3c(ccc4nc(-c5ccccc5)sc43)c21 ; CC1(C)c2cc(-c3nc(-c4ccccc4)nc(-c4cccc5oc6ccccc6c45)n3)ccc2-c2ccc3c(ccc4nc(-c5ccccc5)sc43)c21]

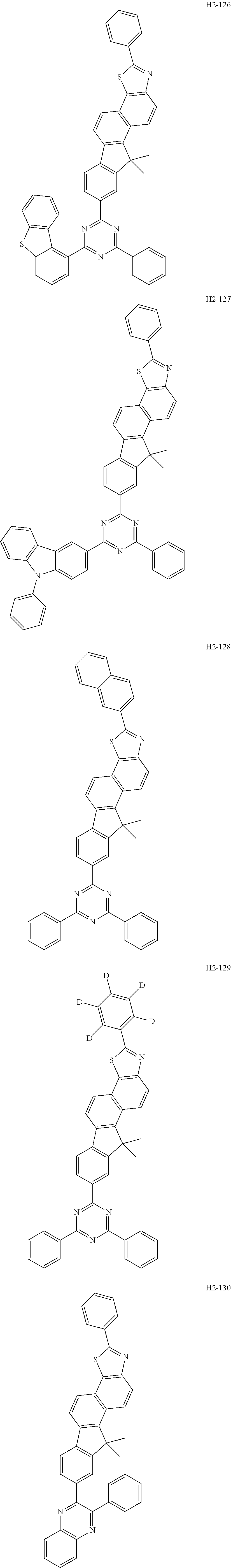

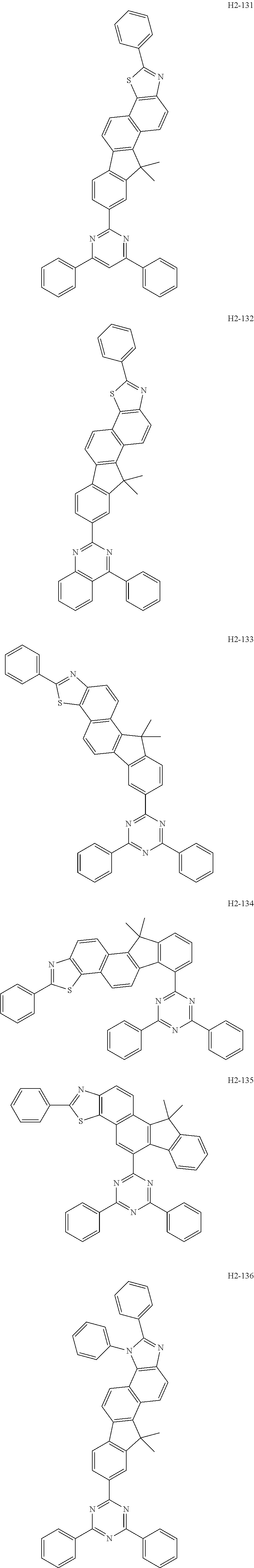

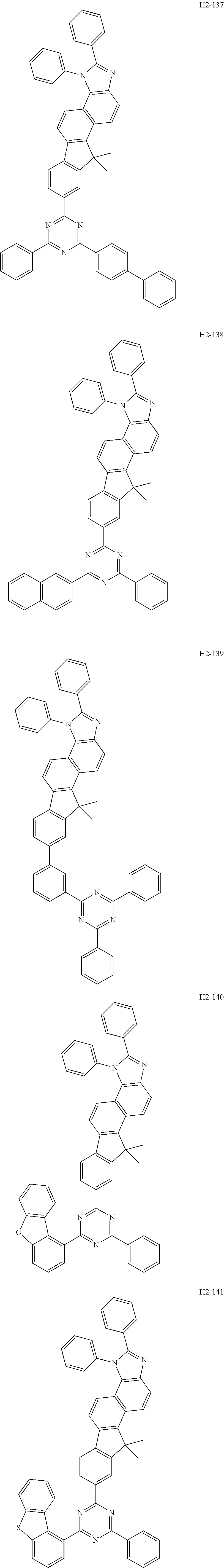

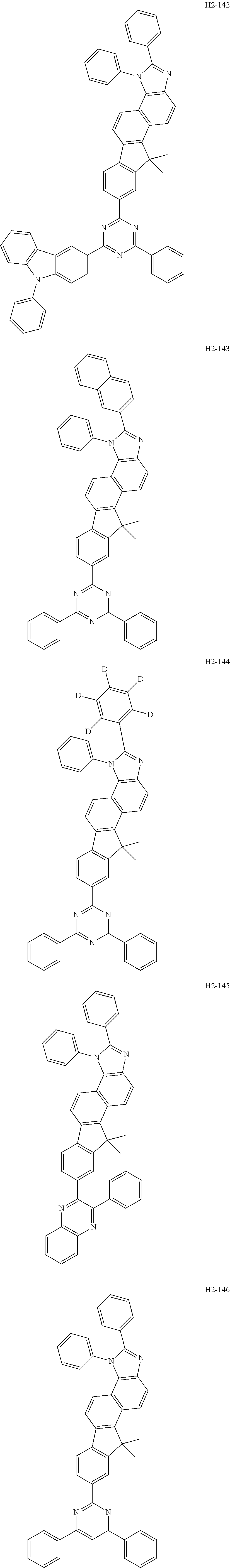

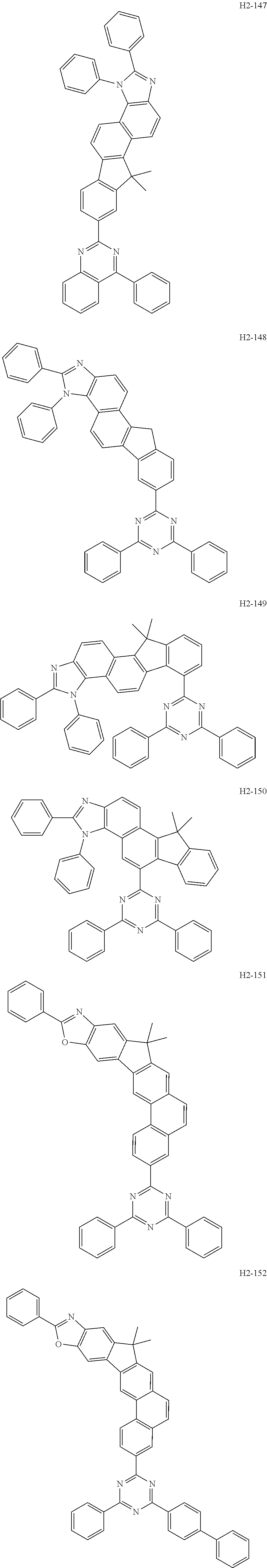

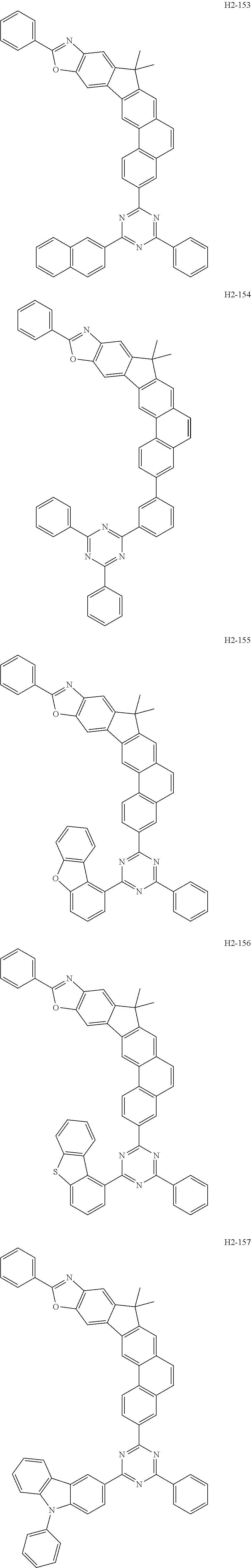 [SMILES: CC1(C)c2cc3ccc4cc(-c5nc(-c6ccccc6)nc(-c6ccc7ccccc7c6)n5)ccc4c3cc2-c2cc3oc(-c4ccccc4)nc3cc21 ; CC1(C)c2cc3ccc4cc(-c5cccc(-c6nc(-c7ccccc7)nc(-c7ccccc7)n6)c5)ccc4c3cc2-c2cc3oc(-c4ccccc4)nc3cc21 ; CC1(C)c2cc3ccc4cc(-c5nc(-c6ccccc6)nc(-c6cccc7oc8ccccc8c67)n5)ccc4c3cc2-c2cc3oc(-c4ccccc4)nc3cc21 ; CC1(C)c2cc3ccc4cc(-c5nc(-c6ccccc6)nc(-c6cccc7sc8ccccc8c67)n5)ccc4c3cc2-c2cc3oc(-c4ccccc4)nc3cc21 ; CC1(C)c2cc3ccc4cc(-c5nc(-c6ccccc6)nc(-c6ccc7c(c6)c6ccccc6n7-c6ccccc6)n5)ccc4c3cc2-c2cc3oc(-c4ccccc4)nc3cc21]

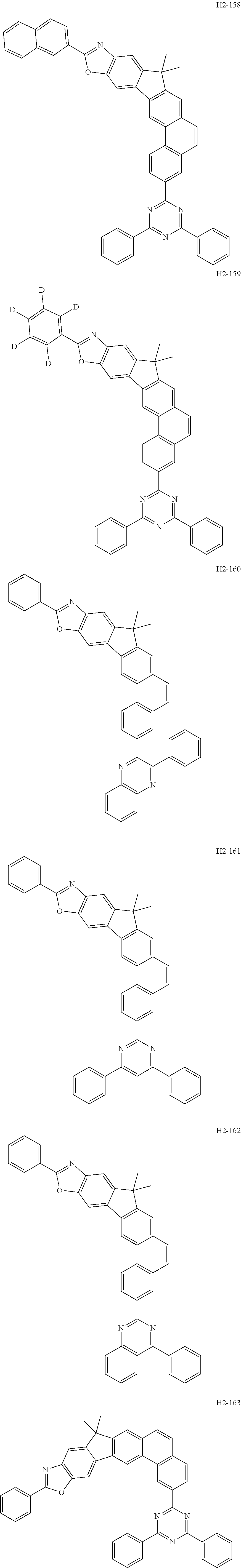

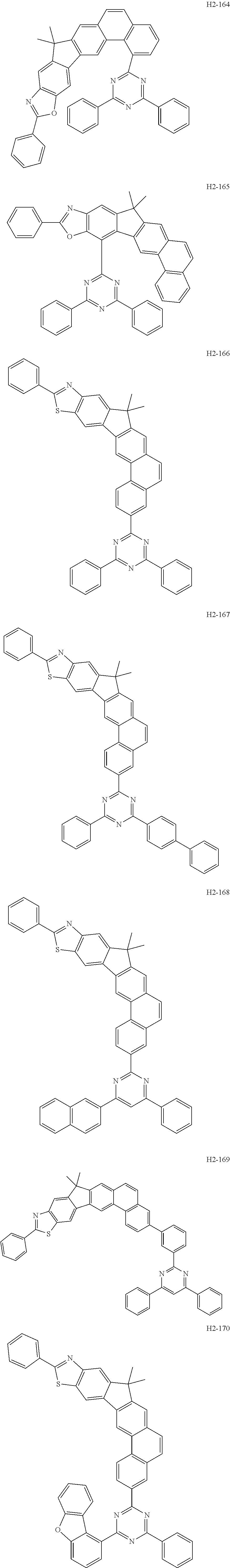

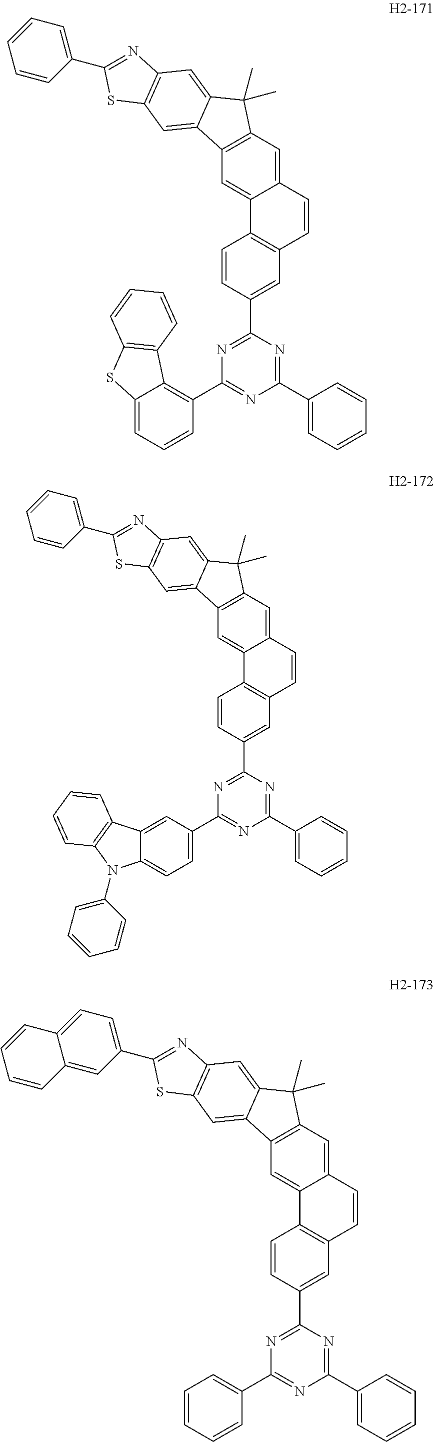 [SMILES: CC1(C)c2cc3ccc4cc(-c5nc(-c6ccccc6)nc(-c6cccc7sc8ccccc8c67)n5)ccc4c3cc2-c2cc3sc(-c4ccccc4)nc3cc21 ; CC1(C)c2cc3ccc4cc(-c5nc(-c6ccccc6)nc(-c6ccc7c(c6)c6ccccc6n7-c6ccccc6)n5)ccc4c3cc2-c2cc3sc(-c4ccccc4)nc3cc21 ; CC1(C)c2cc3ccc4cc(-c5nc(-c6ccccc6)nc(-c6ccccc6)n5)ccc4c3cc2-c2cc3sc(-c4ccc5ccccc5c4)nc3cc21]

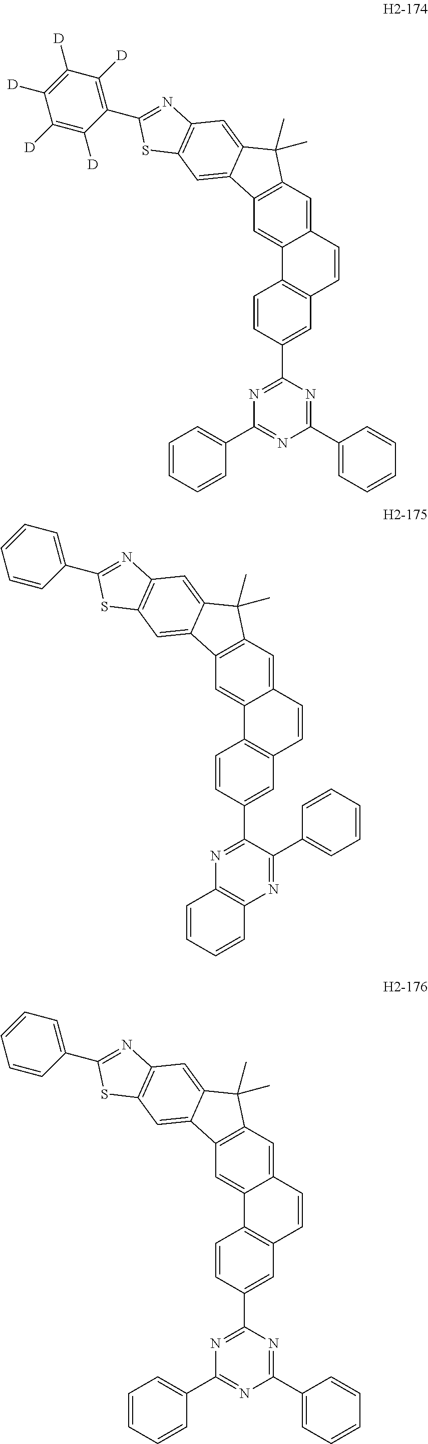 [SMILES: [2H]c1c([2H])c([2H])c(-c2nc3cc4c(cc3s2)-c2cc3c(ccc5cc(-c6nc(-c7ccccc7)nc(-c7ccccc7)n6)ccc53)cc2C4(C)C)c([2H])c1[2H] ; CC1(C)c2cc3ccc4cc(-c5nc6ccccc6nc5-c5ccccc5)ccc4c3cc2-c2cc3sc(-c4ccccc4)nc3cc21 ; CC1(C)c2cc3ccc4cc(-c5nc(-c6ccccc6)nc(-c6ccccc6)n5)ccc4c3cc2-c2cc3sc(-c4ccccc4)nc3cc21]

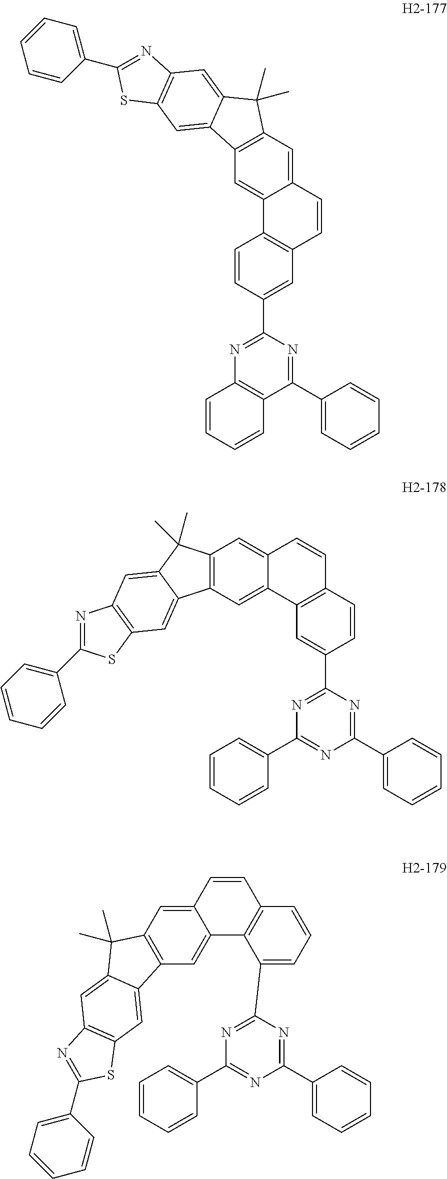 [SMILES: CC1(C)c2cc3ccc4cc(-c5nc(-c6ccccc6)c6ccccc6n5)ccc4c3cc2-c2cc3sc(-c4ccccc4)nc3cc21 ; CC1(C)c2cc3ccc4ccc(-c5nc(-c6ccccc6)nc(-c6ccccc6)n5)cc4c3cc2-c2cc3sc(-c4ccccc4)nc3cc21 ; CC1(C)c2cc3ccc4cccc(-c5nc(-c6ccccc6)nc(-c6ccccc6)n5)c4c3cc2-c2cc3sc(-c4ccccc4)nc3cc21]

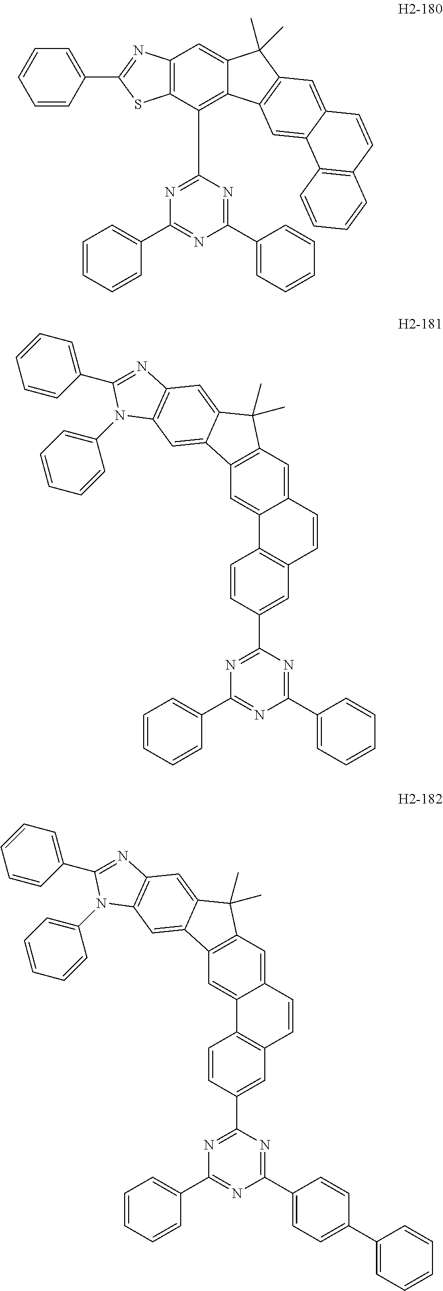 [SMILES: CC1(C)c2cc3ccc4ccccc4c3cc2-c2c1cc1nc(-c3ccccc3)sc1c2-c1nc(-c2ccccc2)nc(-c2ccccc2)n1 ; CC1(C)c2cc3ccc4cc(-c5nc(-c6ccccc6)nc(-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccc(-c7ccccc7)cc6)n5)ccc4c3cc2-c2cc3c(cc21)nc(-c1ccccc1)n3-c1ccccc1]

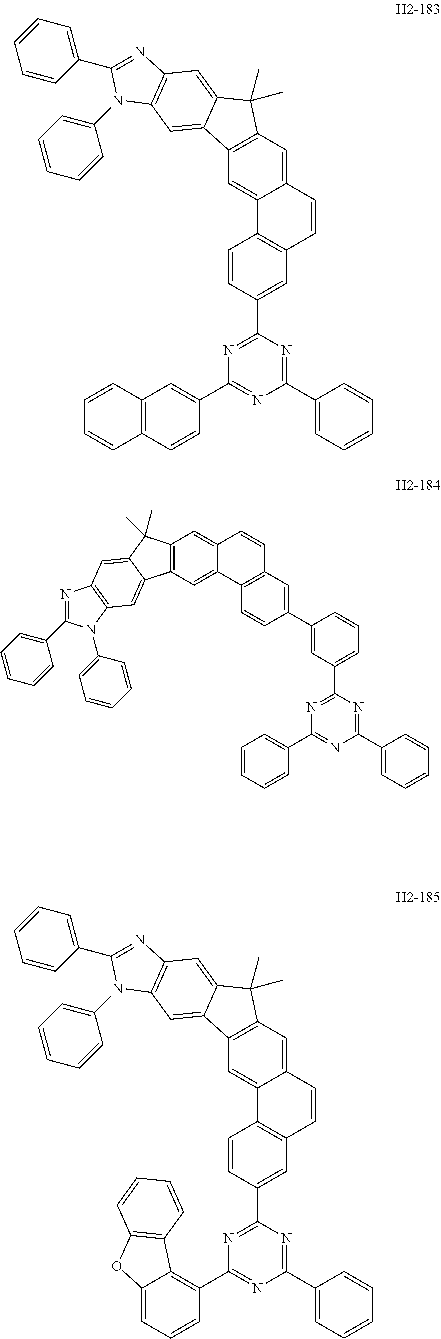 [SMILES: CC1(C)c2cc3ccc4cc(-c5nc(-c6ccccc6)nc(-c6ccc7ccccc7c6)n5)ccc4c3cc2-c2cc3c(cc21)nc(-c1ccccc1)n3-c1ccccc1 ; CC1(C)c2cc3ccc4cc(-c5cccc(-c6nc(-c7ccccc7)nc(-c7ccccc7)n6)c5)ccc4c3cc2-c2cc3c(cc21)nc(-c1ccccc1)n3-c1ccccc1 ; CC1(C)c2cc3ccc4cc(-c5nc(-c6ccccc6)nc(-c6cccc7oc8ccccc8c67)n5)ccc4c3cc2-c2cc3c(cc21)nc(-c1ccccc1)n3-c1ccccc1]

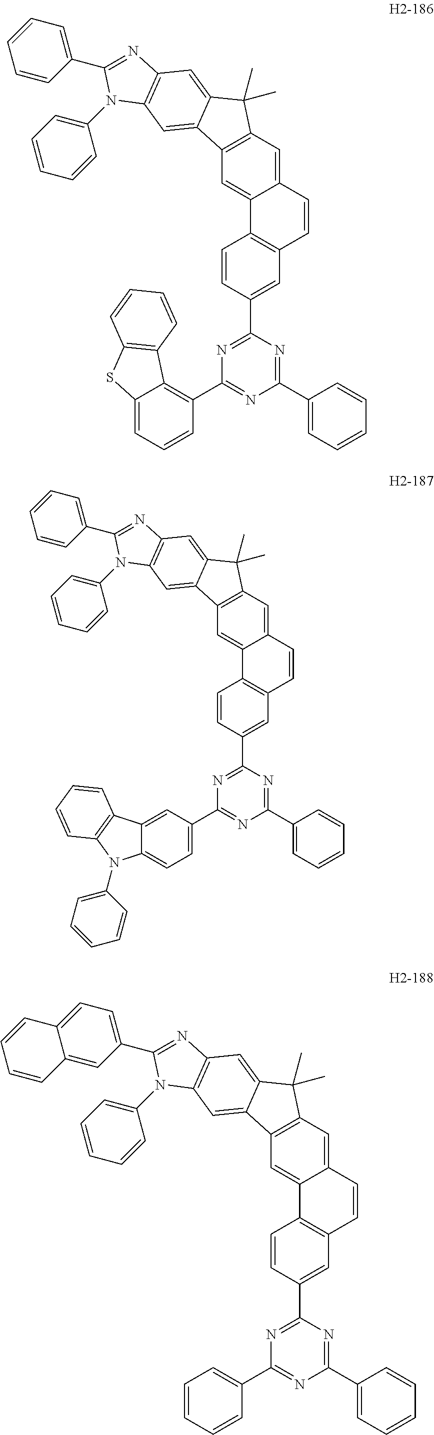 [SMILES: CC1(C)c2cc3ccc4cc(-c5nc(-c6ccccc6)nc(-c6cccc7sc8ccccc8c67)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccc7c(c6)c6ccccc6n7-c6ccccc6)n5)ccc4c3cc2-c2cc3c(cc21)nc(-c1ccccc1)n3-c1ccccc1 ; CC1(C)c2cc3ccc4cc(-c5nc(-c6ccccc6)nc(-c6ccccc6)n5)ccc4c3cc2-c2cc3c(cc21)nc(-c1ccc2ccccc2c1)n3-c1ccccc1]

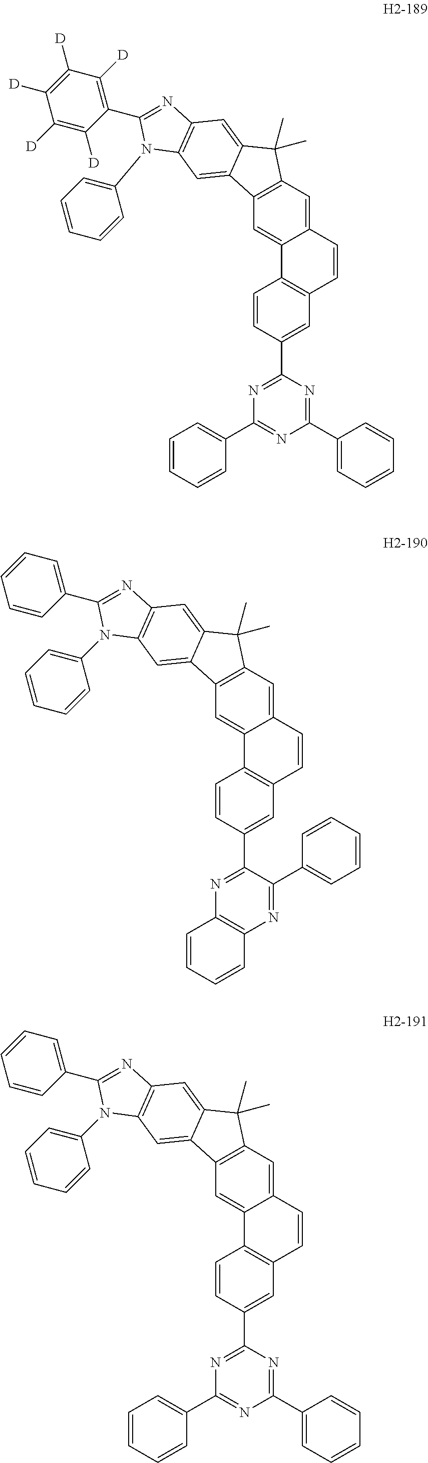 [SMILES: [2H]c1c([2H])c([2H])c(-c2nc3cc4c(cc3n2-c2ccccc2)-c2cc3c(ccc5cc(-c6nc(-c7ccccc7)nc(-c7ccccc7)n6)ccc53)cc2C4(C)C)c([2H])c1[2H] ; CC1(C)c2cc3ccc4cc(-c5nc6ccccc6nc5-c5ccccc5)ccc4c3cc2-c2cc3c(cc21)nc(-c1ccccc1)n3-c1ccccc1 ; CC1(C)c2cc3ccc4cc(-c5nc(-c6ccccc6)nc(-c6ccccc6)n5)ccc4c3cc2-c2cc3c(cc21)nc(-c1ccccc1)n3-c1ccccc1]

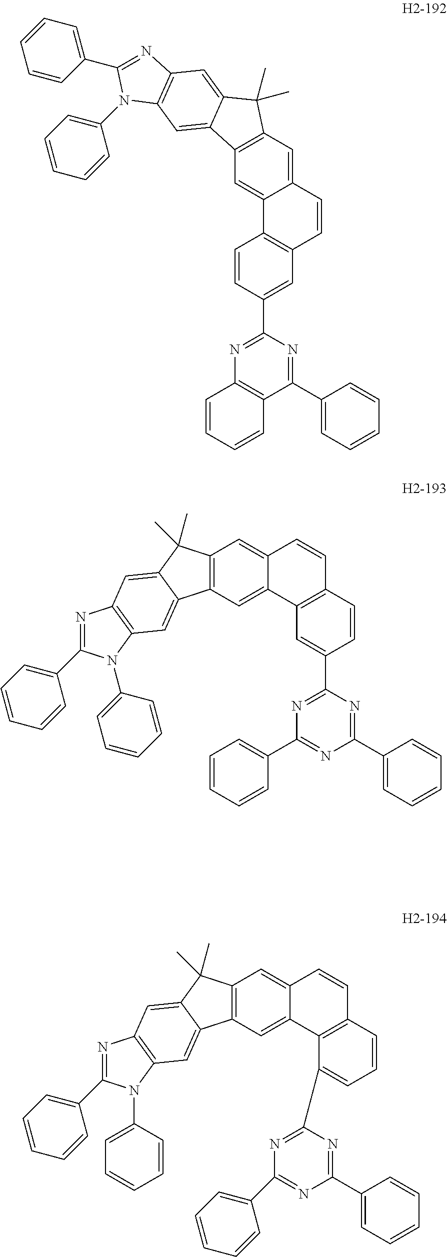 [SMILES: CC1(C)c2cc3ccc4cc(-c5nc(-c6ccccc6)c6ccccc6n5)ccc4c3cc2-c2cc3c(cc21)nc(-c1ccccc1)n3-c1ccccc1 ; CC1(C)c2cc3ccc4ccc(-c5nc(-c6ccccc6)nc(-c6ccccc6)n5)cc4c3cc2-c2cc3c(cc21)nc(-c1ccccc1)n3-c1ccccc1 ; CC1(C)c2cc3ccc4cccc(-c5nc(-c6ccccc6)nc(-c6ccccc6)n5)c4c3cc2-c2cc3c(cc21)nc(-c1ccccc1)n3-c1ccccc1]

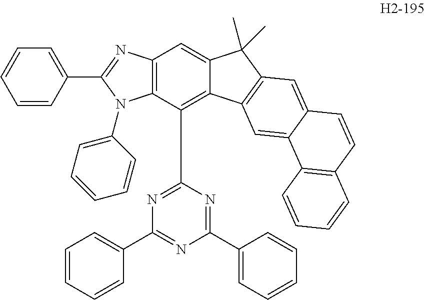 [SMILES: CC1(C)c2cc3ccc4ccccc4c3cc2-c2c1cc1nc(-c3ccccc3)n(-c3ccccc3)c1c2-c1nc(-c2ccccc2)nc(-c2ccccc2)n1]

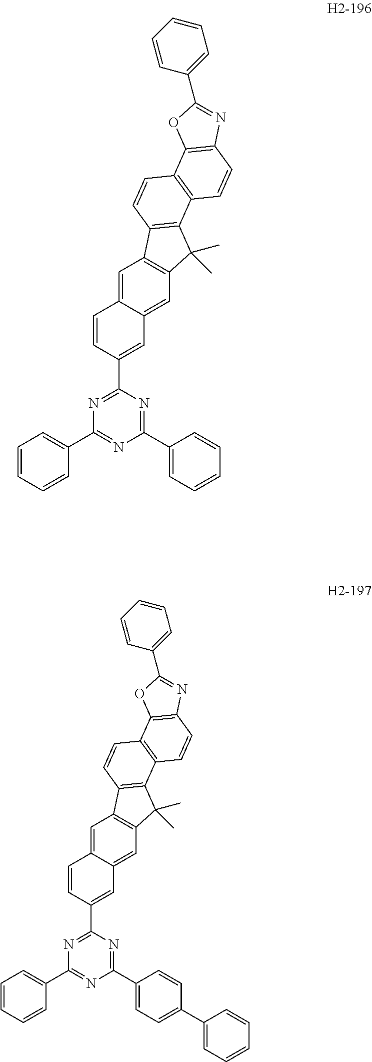 [SMILES: CC1(C)c2cc3cc(-c4nc(-c5ccccc5)nc(-c5ccccc5)n4)ccc3cc2-c2ccc3c(ccc4nc(-c5ccccc5)oc43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)oc43)c21]

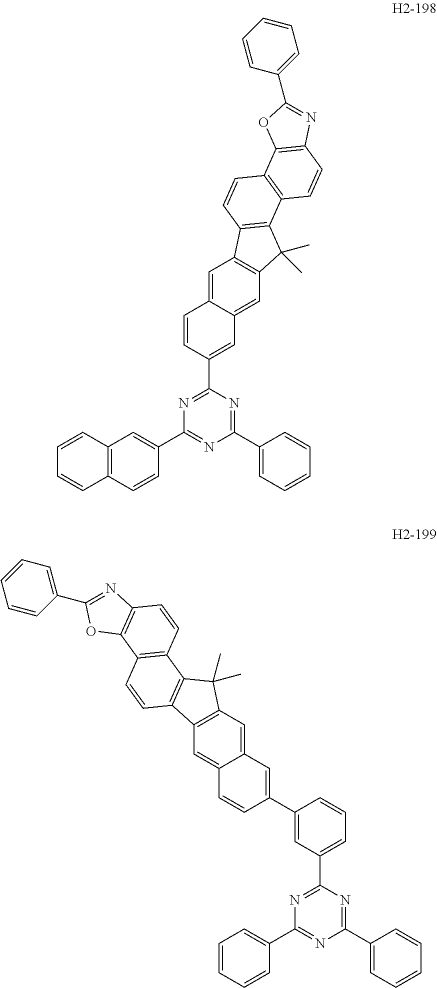 [SMILES: CC1(C)c2cc3cc(-c4nc(-c5ccccc5)nc(-c5ccc6ccccc6c5)n4)ccc3cc2-c2ccc3c(ccc4nc(-c5ccccc5)oc43)c21 ; CC1(C)c2cc3cc(-c4cccc(-c5nc(-c6ccccc6)nc(-c6ccccc6)n5)c4)ccc3cc2-c2ccc3c(ccc4nc(-c5ccccc5)oc43)c21]

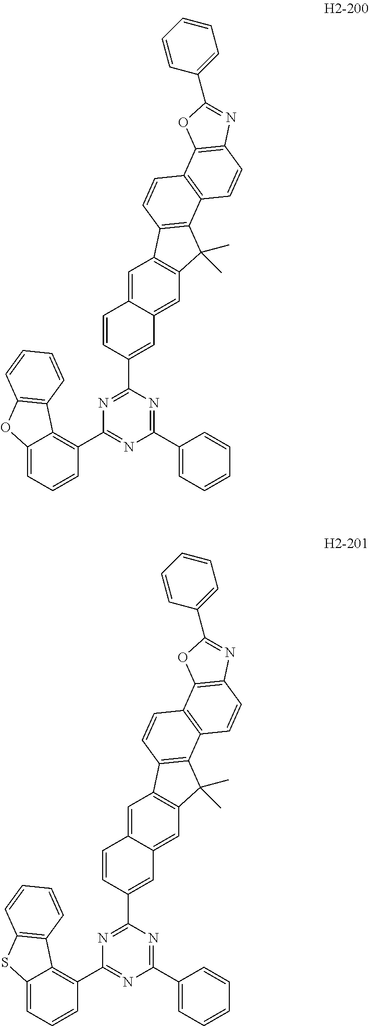 [SMILES: CC1(C)c2cc3cc(-c4nc(-c5ccccc5)nc(-c5cccc6oc7ccccc7c56)n4)ccc3cc2-c2ccc3c(ccc4nc(-c5ccccc5)oc43)c21 ; CC1(C)c2cc3cc(-c4nc(-c5ccccc5)nc(-c5cccc6sc7ccccc7c56)n4)ccc3cc2-c2ccc3c(ccc4nc(-c5ccccc5)oc43)c21]

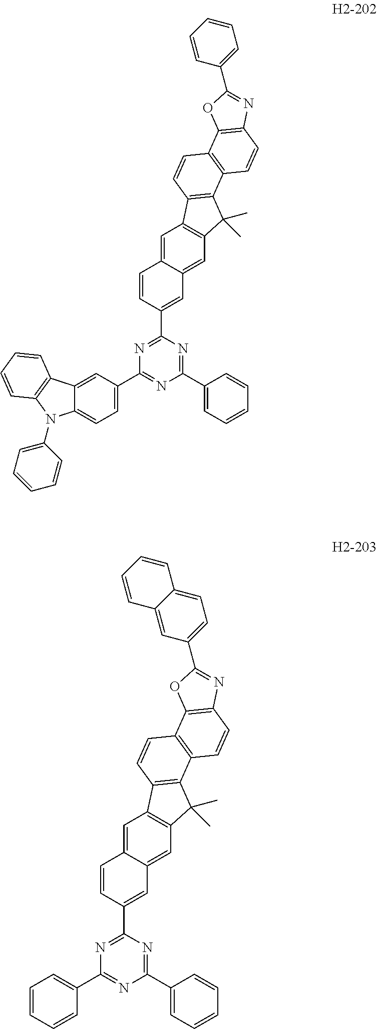 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)oc43)c21 ; CC1(C)c2cc3cc(-c4nc(-c5ccccc5)nc(-c5ccccc5)n4)ccc3cc2-c2ccc3c(ccc4nc(-c5ccc6ccccc6c5)oc43)c21]

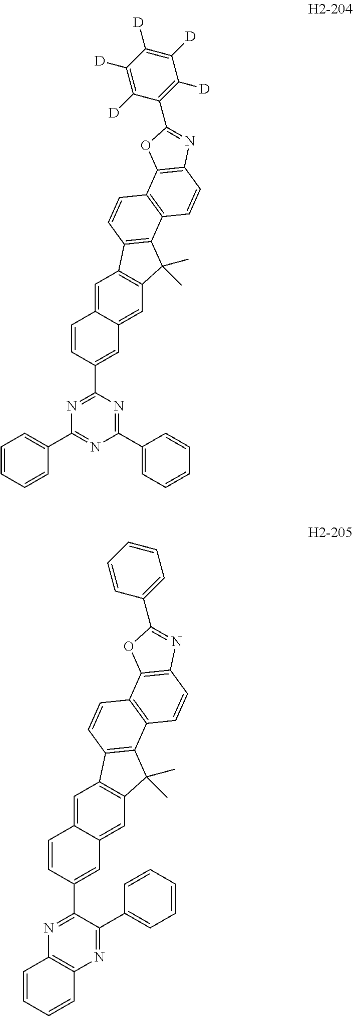 [SMILES: [2H]c1c([2H])c([2H])c(-c2nc3ccc4c5c(ccc4c3o2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)oc43)c21]

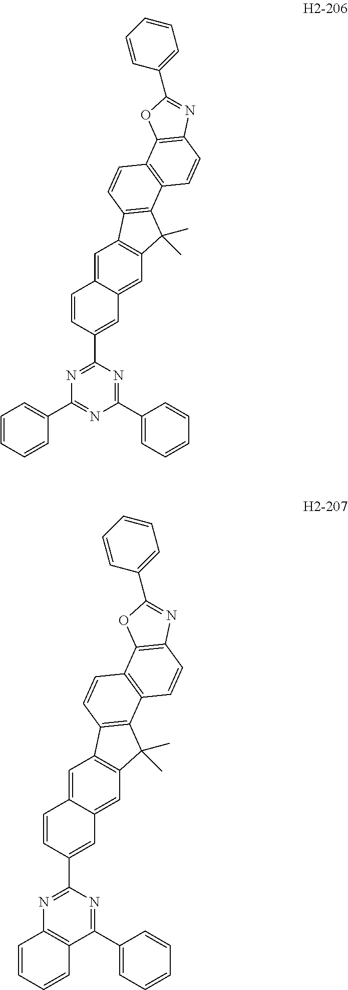 [SMILES: CC1(C)c2cc3cc(-c4nc(-c5ccccc5)nc(-c5ccccc5)n4)ccc3cc2-c2ccc3c(ccc4nc(-c5ccccc5)oc43)c21 ; CC1(C)c2cc3cc(-c4nc(-c5ccccc5)c5ccccc5n4)ccc3cc2-c2ccc3c(ccc4nc(-c5ccccc5)oc43)c21]

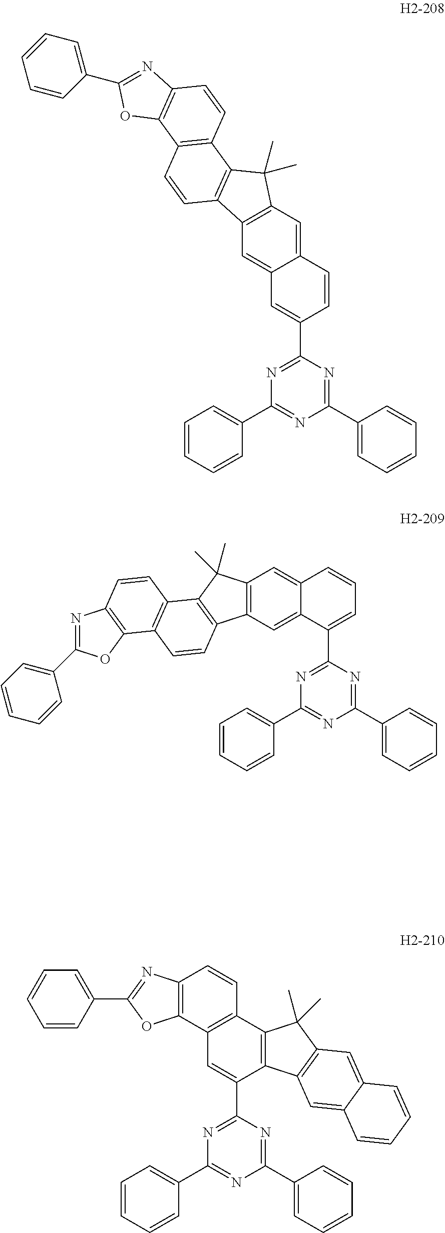 [SMILES: CC1(C)c2cc3ccc(-c4nc(-c5ccccc5)nc(-c5ccccc5)n4)cc3cc2-c2ccc3c(ccc4nc(-c5ccccc5)oc43)c21 ; CC1(C)c2cc3cccc(-c4nc(-c5ccccc5)nc(-c5ccccc5)n4)c3cc2-c2ccc3c(ccc4nc(-c5ccccc5)oc43)c21 ; CC1(C)c2cc3ccccc3cc2-c2c(-c3nc(-c4ccccc4)nc(-c4ccccc4)n3)cc3c(ccc4nc(-c5ccccc5)oc43)c21]

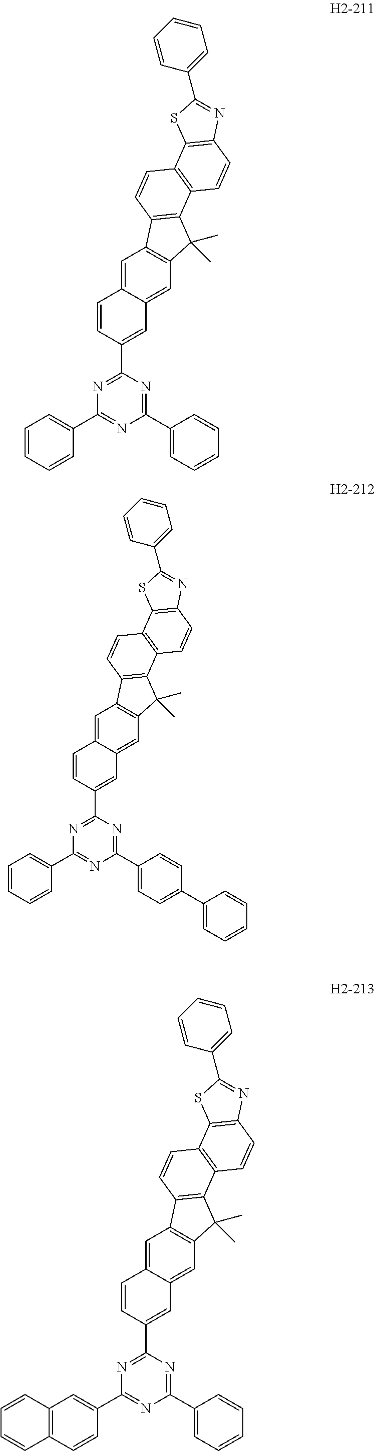 [SMILES: CC1(C)c2cc3cc(-c4nc(-c5ccccc5)nc(-c5ccccc5)n4)ccc3cc2-c2ccc3c(ccc4nc(-c5ccccc5)sc43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)sc43)c21 ; CC1(C)c2cc3cc(-c4nc(-c5ccccc5)nc(-c5ccc6ccccc6c5)n4)ccc3cc2-c2ccc3c(ccc4nc(-c5ccccc5)sc43)c21]

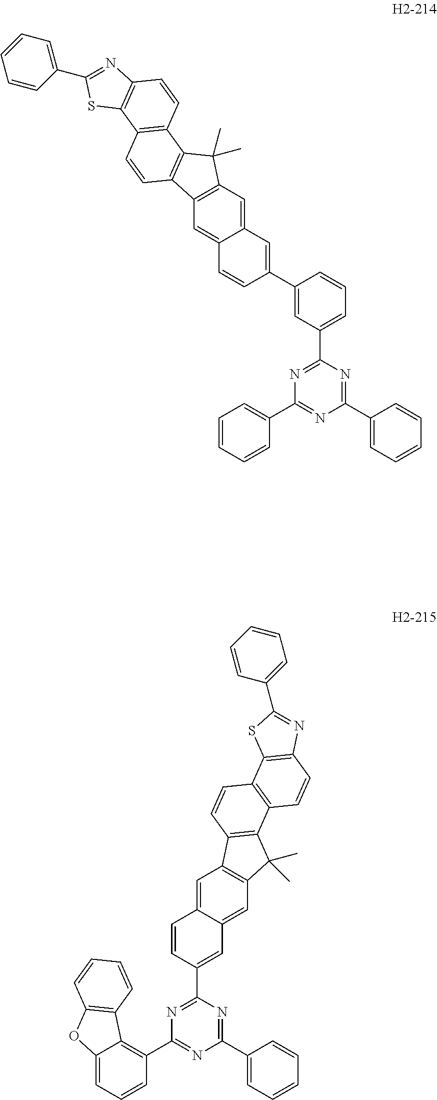 [SMILES: CC1(C)c2cc3cc(-c4cccc(-c5nc(-c6ccccc6)nc(-c6ccccc6)n5)c4)ccc3cc2-c2ccc3c(ccc4nc(-c5ccccc5)sc43)c21 ; CC1(C)c2cc3cc(-c4nc(-c5ccccc5)nc(-c5cccc6oc7ccccc7c56)n4)ccc3cc2-c2ccc3c(ccc4nc(-c5ccccc5)sc43)c21]

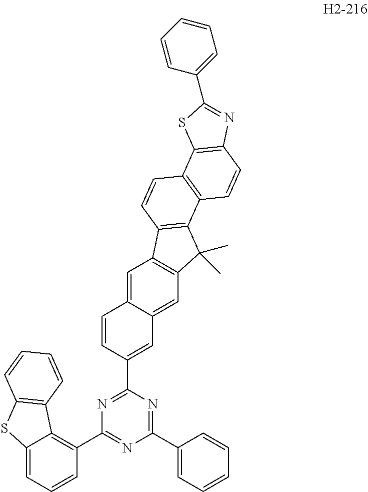 [SMILES: CC1(C)c2cc3cc(-c4nc(-c5ccccc5)nc(-c5cccc6sc7ccccc7c56)n4)ccc3cc2-c2ccc3c(ccc4nc(-c5ccccc5)sc43)c21]

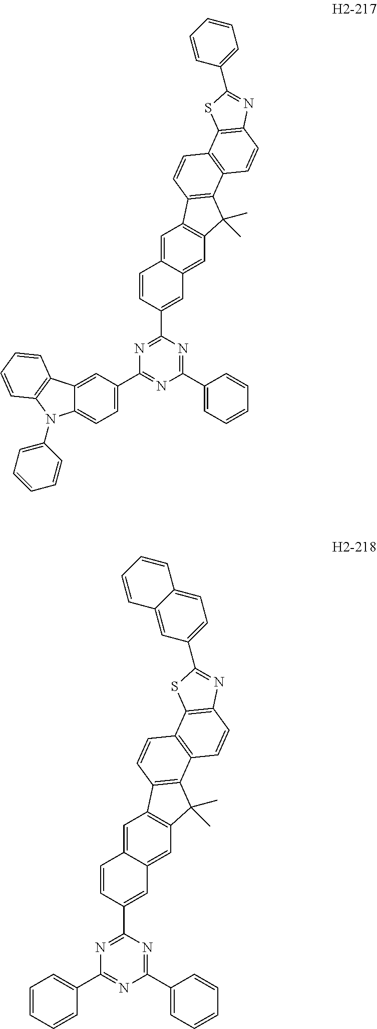 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)sc43)c21 ; CC1(C)c2cc3cc(-c4nc(-c5ccccc5)nc(-c5ccccc5)n4)ccc3cc2-c2ccc3c(ccc4nc(-c5ccc6ccccc6c5)sc43)c21]

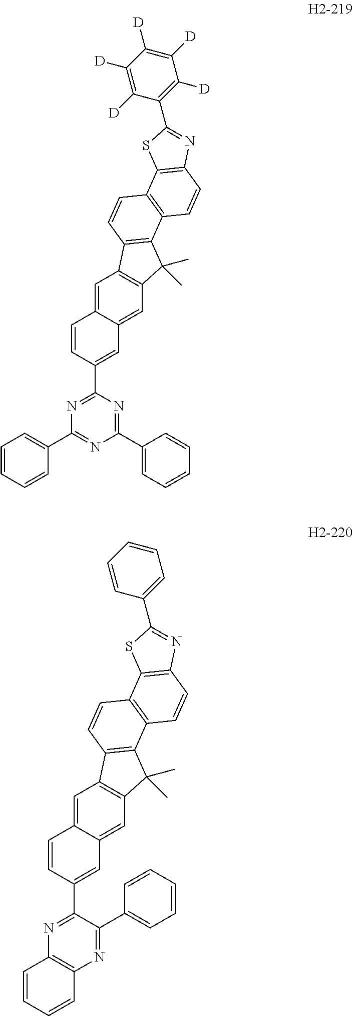 [SMILES: [2H]c1c([2H])c([2H])c(-c2nc3ccc4c5c(ccc4c3s2)-c2cc3ccc(-c4nc(-c6ccccc6)nc(-c6ccccc6)n4)cc3cc2C5(C)C)c([2H])c1[2H] ; CC1(C)c2cc3cc(-c4nc5ccccc5nc4-c4ccccc4)ccc3cc2-c2ccc3c(ccc4nc(-c5ccccc5)sc43)c21]

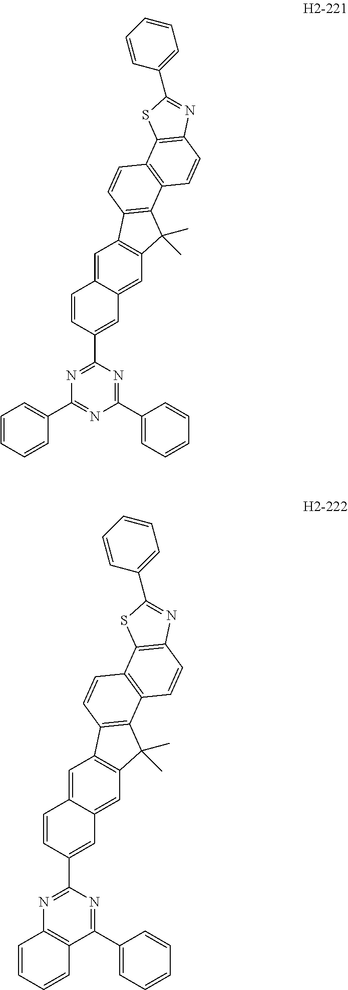 [SMILES: CC1(C)c2cc3cc(-c4nc(-c5ccccc5)nc(-c5ccccc5)n4)ccc3cc2-c2ccc3c(ccc4nc(-c5ccccc5)sc43)c21 ; CC1(C)c2cc3cc(-c4nc(-c5ccccc5)c5ccccc5n4)ccc3cc2-c2ccc3c(ccc4nc(-c5ccccc5)sc43)c21]

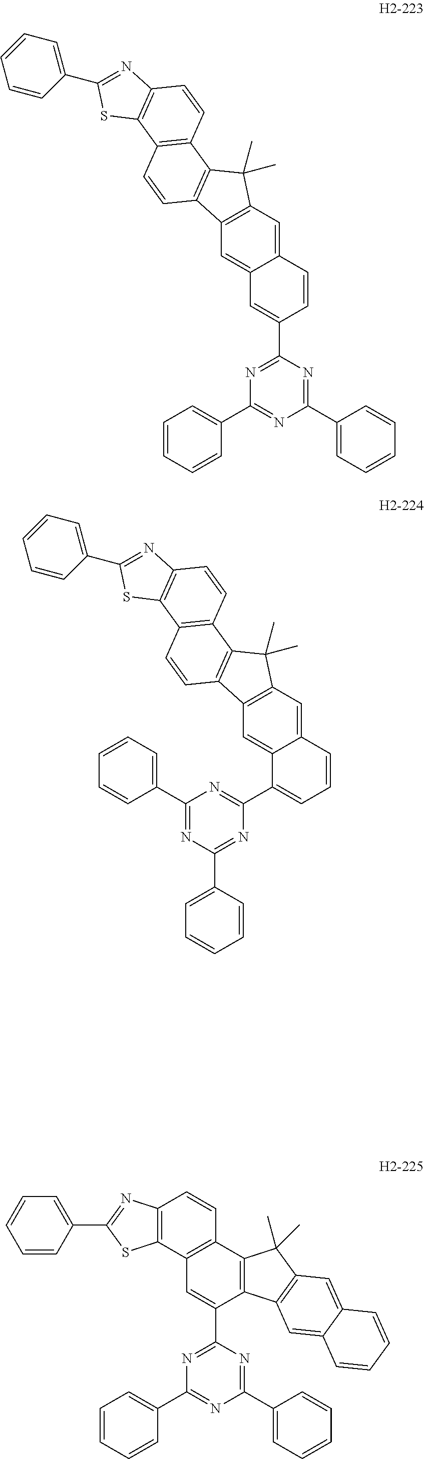 [SMILES: CC1(C)c2cc3ccc(-c4nc(-c5ccccc5)nc(-c5ccccc5)n4)cc3cc2-c2ccc3c(ccc4nc(-c5ccccc5)sc43)c21 ; CC1(C)c2cc3cccc(-c4nc(-c5ccccc5)nc(-c5ccccc5)n4)c3cc2-c2ccc3c(ccc4nc(-c5ccccc5)sc43)c21 ; CC1(C)c2cc3ccccc3cc2-c2c(-c3nc(-c4ccccc4)nc(-c4ccccc4)n3)cc3c(ccc4nc(-c5ccccc5)sc43)c21]

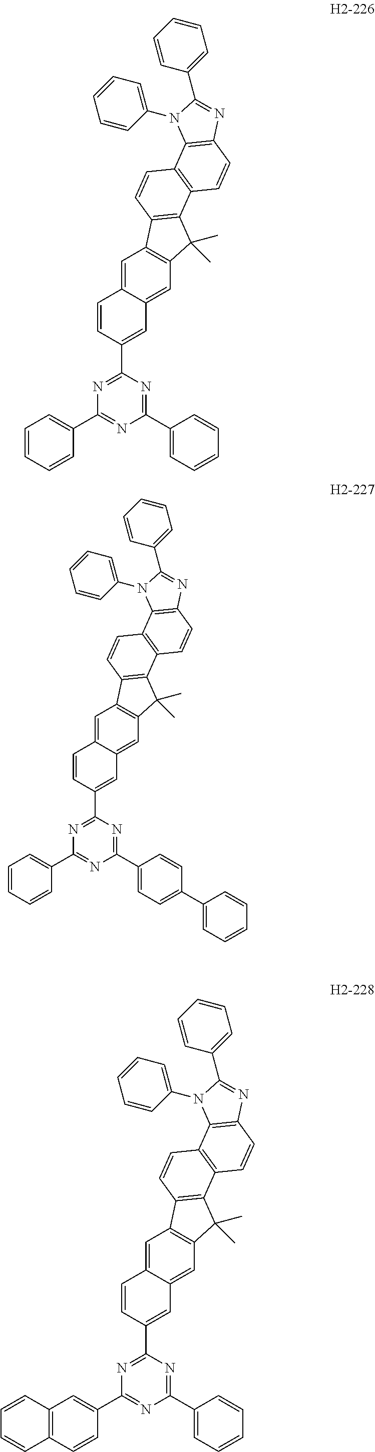 [SMILES: CC1(C)c2cc3cc(-c4nc(-c5ccccc5)nc(-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccc(-c6ccccc6)cc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccc6ccccc6c5)n4)ccc3cc2-c2ccc3c(ccc4nc(-c5ccccc5)n(-c5ccccc5)c43)c21]

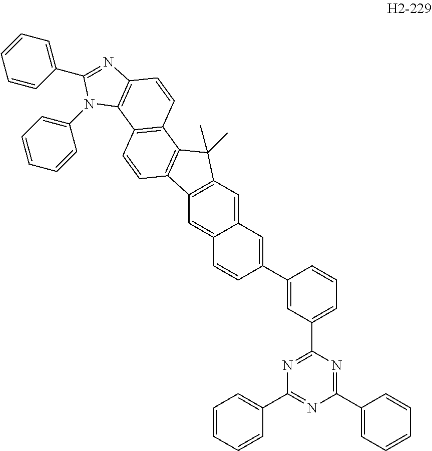 [SMILES: CC1(C)c2cc3cc(-c4cccc(-c5nc(-c6ccccc6)nc(-c6ccccc6)n5)c4)ccc3cc2-c2ccc3c(ccc4nc(-c5ccccc5)n(-c5ccccc5)c43)c21]

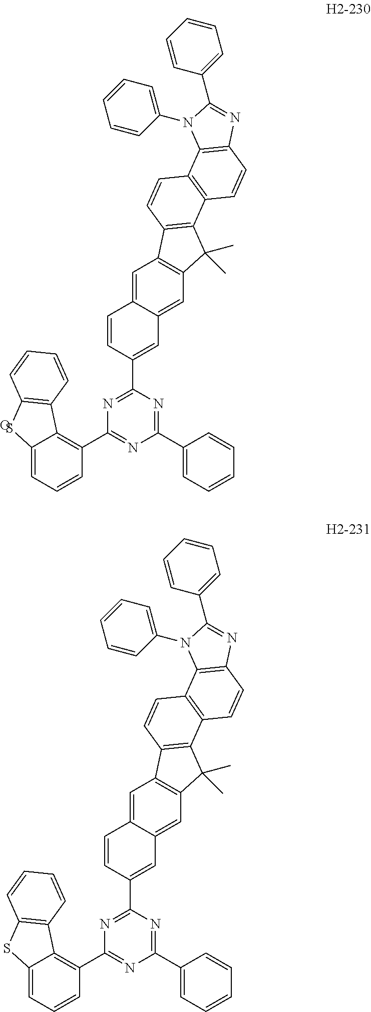

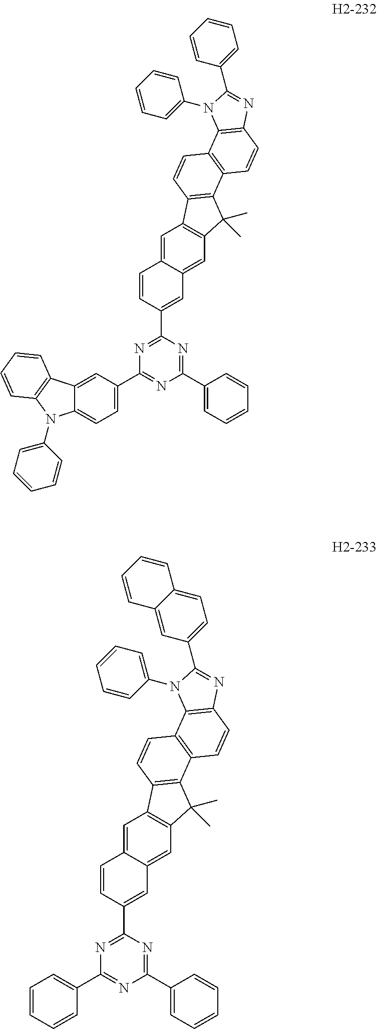 [SMILES: CC1(C)c2cc3cc(-c4nc(-c5ccccc5)nc(-c5ccc6c(c5)c5ccccc5n6-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)nc(-c5ccccc5)n4)ccc3cc2-c2ccc3c(ccc4nc(-c5ccc6ccccc6c5)n(-c5ccccc5)c43)c21]

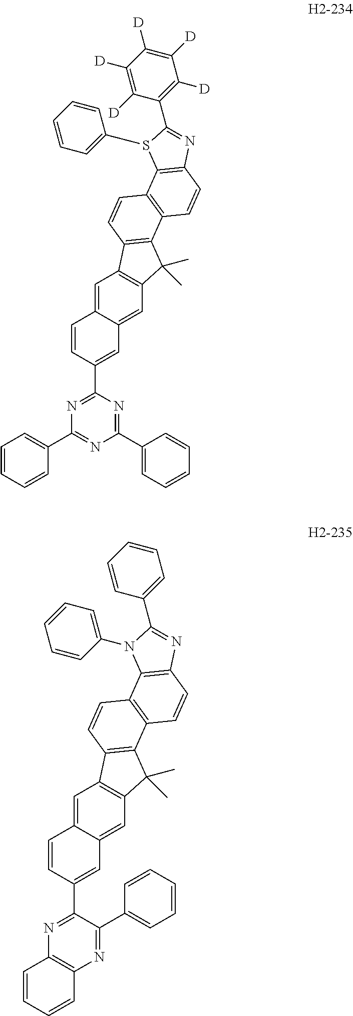

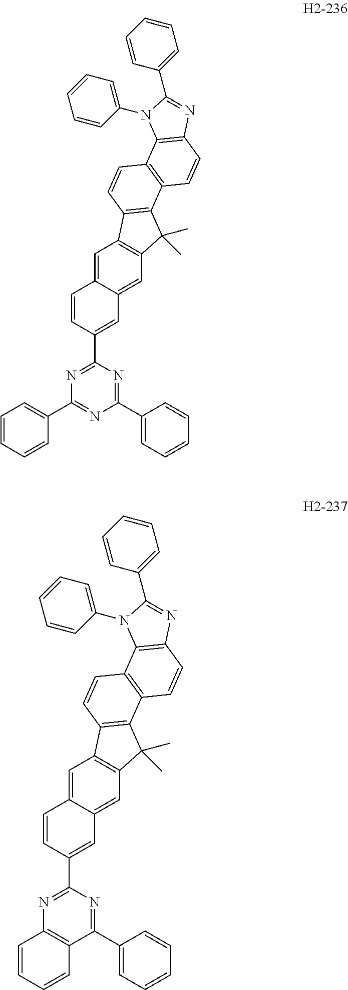 [SMILES: CC1(C)c2cc3cc(-c4nc(-c5ccccc5)nc(-c5ccccc5)n4)ccc3cc2-c2ccc3c(ccc4nc(-c5ccccc5)n(-c5ccccc5)c43)c21 ; CC1(C)c2cc3cc(-c4nc(-c5ccccc5)c5ccccc5n4)ccc3cc2-c2ccc3c(ccc4nc(-c5ccccc5)n(-c5ccccc5)c43)c21]

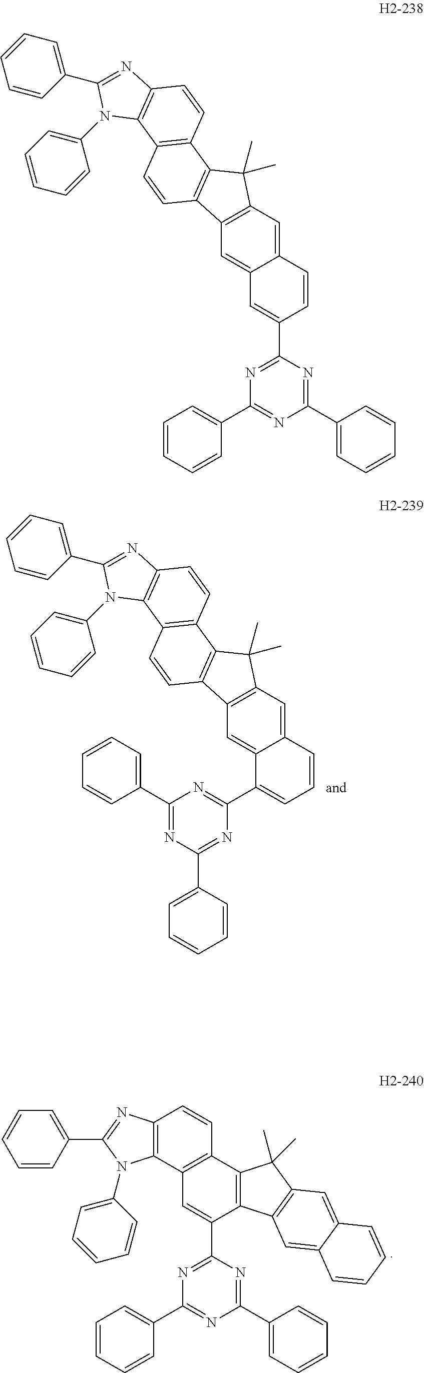 [SMILES: CC1(C)c2cc3ccc(-c4nc(-c5ccccc5)nc(-c5ccccc5)n4)cc3cc2-c2ccc3c(ccc4nc(-c5ccccc5)n(-c5ccccc5)c43)c21 ; CC1(C)c2cc3cccc(-c4nc(-c5ccccc5)nc(-c5ccccc5)n4)c3cc2-c2ccc3c(ccc4nc(-c5ccccc5)n(-c5ccccc5)c43)c21 ; "and" ; CC1(C)c2cc3ccccc3cc2-c2c(-c3nc(-c4ccccc4)nc(-c4ccccc4)n3)cc3c(ccc4nc(-c5ccccc5)n(-c5ccccc5)c43)c21 ; "."]

15. An organic electroluminescent material comprising the organic electroluminescent compound according to claim 10.

16. An organic electroluminescent device comprising the organic electroluminescent compound according to claim 10.
